(19) United States
(12) Patent Application Publication
(10) Pub. No.: US 2006/0074102 A1
(43) Pub. Date: Apr. 6, 2006

(54) Kinase inhibitors as therapeutic agents

(76) Inventors: Kevin Cusack, Holden, MA (US); Jose-Andres Salmeron-Garcia, Westminster, MA (US); Thomas D. Gordon, Medway, MA (US); Claude E. Barberis, Santeny (FR); Hamish J. Allen, Boylston, MA (US); Agniezka K. Bischoff, Westborough, MA (US); Anna Ericsson, Shrewsbury, MA (US); Michael Friedman, Newton, MA (US); Dawn M. George, Charlton, MA (US); Gregory P. Roth, Woodstock, CT (US); Robert V. Talanian, Harvard, MA (US); Christine Thomas, Northborough, MA (US); Grier A. Wallace, Sterling, MA (US); Neil Wishart, Jefferson, MA (US); Zhengtian Yu, Shrewsbury, MA (US)

Correspondence Address: ABBOTT BIORESEARCH, 100 RESEARCH DRIVE, WORCESTER, MA, 01605-4314, US

(21) Appl. No.: 11/129,624

(22) Filed: May 13, 2005

Related U.S. Application Data
(60) Provisional application No. 60/571,281, filed on May 14, 2004.

(51) Int. Cl. A61K 31/4743; C07D 498/02

(52) U.S. Cl. 514/301; 546/114

(57) ABSTRACT

A compound or pharmaceutically acceptable salts thereof of Formula (I) 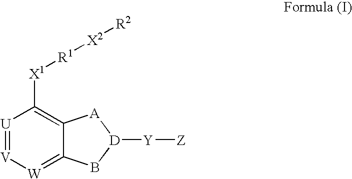 wherein the substituents are as defined herein, which are useful as kinase inhibitors.

### CROSS-REFERENCE TO RELATED APPLICATION

This application claims the benefit of priority to U.S. application No. 60/571,281, filed May 14, 2004.

### BACKGROUND OF THE INVENTION

Protein phosphorylation, at specific amino acid residues, is important for the regulation of many cellular processes including cell cycle progression and division, signal transduction, and apoptosis. The phosphorylation is usually a transfer reaction of the terminal phosphate group from ATP to the protein substrate. The specific structure in the target substrate to which the phosphate is transferred is a tyrosine, serine or threonine residue. Since these amino acid residues are the target structures for the phosphoryl transfer, these protein kinase enzymes are commonly referred to as tyrosine kinases or serine/threonine (S/T) kinases. The phosphorylation reactions, and counteracting phosphatase reactions, on the tyrosine, serine and threonine residues are involved in countless cellular processes that underlie responses to diverse intracellular signals, regulation of cellular functions, and activation or deactivation of cellular processes. A cascade of protein kinases often participate in intracellular signal transduction and are necessary for the realization of cellular processes. Because of their ubiquity in these processes, the protein kinases can be found as an integral part of the plasma membrane or as cytoplasmic enzymes or localized in the nucleus, often as components of enzyme complexes. In many instances, these protein kinases are an essential element of enzyme and structural protein complexes that determine where and when a cellular process occurs within a cell. Given the importance and diversity of protein kinase function, it is not surprising that alterations in phosphorylation are associated with many diseases such as cancer, diabetes, inflammation, and hypertension.

The identification of effective small molecules that specifically inhibit protein kinases involved in abnormal or inappropriate cell proliferation, signaling, differentiation, protein production, or metabolism is therefore desirable. In particular, the identification of methods and compounds that specifically inhibit the function of kinases that are involved in immune modulation or proliferative disorders.

The present invention provides novel compounds that inhibit one or more receptor, or non-receptor, tyrosine or S/T kinase.

### SUMMARY OF THE INVENTION

The present invention provides a compound or pharmaceutically acceptable salts thereof having an IC₅₀ of about 20 μM or less in a COT phosphorylation assay in macrophages.

In first embodiment a compound or pharmaceutically acceptable salts thereof according to any of the foregoing inventions wherein said compound also has at least one of the following properties:
a) inhibits pErk signaling resulting from LPS stimulation in a macrophage with an EC₅₀ of about 6 μM or less;
b) inhibits TNF-alpha production resulting from LPS stimulation in macrophages with an EC₅₀ of about 20 μM or less;
c) inhibits IL-1 production resulting from LPS stimulation in macrophages with an EC₅₀ of about 20 μM or less;
d) inhibits TNF-alpha production resulting from LPS stimulation in macrophages in the presence of plasma with an EC₅₀ of about 100 μM or less;
e) inhibits IL-1 production resulting from LPS stimulation in macrophages in the presence of plasma with an EC₅₀ of about 100 μM or less;
f) inhibits LPS induced TNF-alpha in a mouse with an ED₅₀ of about 100 mg/kg or less;
g) inhibits LPS induced IL-1 in a mouse with an ED₅₀ of about 100 mg/kg or less; or
h) inhibits collagen induced arthritis in a mouse with an ED₅₀ of about 500 mg/kg/day or less.

In a second embodiment, the invention provides a compound or pharmaceutically acceptable salts thereof according to any of the foregoing inventions wherein said compound also inhibits pErk signaling resulting from LPS stimulation in a macrophage with an EC₅₀ of about 6 μM or less.

In a third embodiment, the invention provides a compound or pharmaceutically acceptable salts thereof according to any of the foregoing inventions wherein said compound also inhibits TNF-alpha production resulting from LPS stimulation in macrophages with an EC₅₀ of about 20 μM or less.

In a fourth embodiment, the invention provides a compound or pharmaceutically acceptable salts thereof according to any of the foregoing inventions wherein said compound also inhibits IL-1 production resulting from LPS stimulation in macrophages with an EC₅₀ Of about 20 μM or less.

In a fifth embodiment, the invention provides a compound or pharmaceutically acceptable salts thereof according to any of the foregoing inventions wherein said compound also inhibits TNF-alpha production resulting from LPS stimulation in macrophages in the presence of plasma with an EC₅₀ of about 100 μM or less.

In a sixth embodiment, the invention provides a compound or pharmaceutically acceptable salts thereof according to any of the foregoing inventions wherein said compound also inhibits IL-1 production resulting from LPS stimulation in macrophages in the presence of plasma with an EC₅₀ of about 100 μM or less.

In an seventh embodiment, the invention provides a compound or pharmaceutically acceptable salts thereof according to any of the foregoing inventions wherein said compound also inhibits LPS induced TNF-alpha in a mouse with an ED₅₀ of about 100 mg/kg or less.

In a eighth embodiment, the invention provides a compound or pharmaceutically acceptable salts thereof according to any of the foregoing inventions wherein said compound also inhibits LPS induced EL-1 in a mouse with an ED₅₀ of about 100 mg/kg or less.

In a ninth embodiment, the invention provides a compound or pharmaceutically acceptable salts thereof according to any of the foregoing inventions wherein said compound also inhibits collagen induced arthritis in a mouse with an ED₅₀ of about 500 mg/kg/day or less.

In a tenth embodiment, the invention provides a compound or pharmaceutically acceptable salts thereof having an IC₅₀ of about 20 μM or less in a COT phosphorylation assay in macrophages and having a moiety of the formula 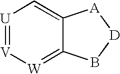
as a component of its complete structure, wherein
A is selected from N, S, O, bond, C═C, C and N;
B is selected from N, S, O, bond, C═C, C and N;
D is selected from C, N, S, O, and C═C;
wherein a double bond is optionally between A and D or B and D;
provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C or N;
V is C or N; and
W is C or N.

In an eleventh embodiment, the invention provides a compound or pharmaceutically acceptable salts thereof according to any of the foregoing inventions wherein the moiety is of the formula

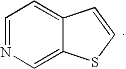

In a twelfth embodiment, the invention provides a compound or pharmaceutically acceptable salts thereof according to any of the foregoing inventions wherein the moiety is of the formula

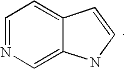

In a thirteenth embodiment, the invention provides a compound or pharmaceutically acceptable salts thereof according to any of the foregoing inventions wherein the moiety is of the formula

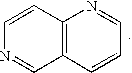

In a fourteenth embodiment, the invention provides a compound or pharmaceutically acceptable salts thereof according to any of the foregoing inventions wherein the moiety is of the formula

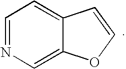

In a fifteenth embodiment, the invention provides a compound or pharmaceutically acceptable salts thereof according to any of the foregoing inventions wherein the moiety is of the formula

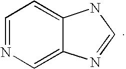

In a sixteenth embodiment, the invention provides a compound or pharmaceutically acceptable salts thereof, having an IC₅₀ of about 5 μM or less in a MK2 HTRF enzyme assay at 5 μM ATP.

In a seventeenth embodiment, the invention provides a compound or pharmaceutically acceptable salts thereof, according to any of the foregoing inventions wherein said compound also has at least one of the following properties:
a) inhibits formation of phospho-Hsp27 resulting from LPS stimulation in a macrophage with an EC₅₀ of about 10 μM or less;
b) inhibits TNF-alpha production resulting from LPS stimulation in macrophages with an EC50 of about 20 μM or less;
c) inhibits TNF-alpha production resulting from LPS stimulation in macrophages in the presence of plasma with an EC₅₀ of about 100 μM or less;
d) inhibits LPS induced TNF-alpha in a mouse with an ED₅₀ of about 100 mg/kg or less; or
e) inhibits collagen induced arthritis in a mouse with an ED₅₀ of about 500 mg/kg/day or less.

In a eighteenth embodiment, the invention provides a compound or pharmaceutically acceptable salts thereof, according to any of the foregoing inventions wherein said compound also inhibits formation of phospho-Hsp27 resulting from LPS stimulation in a macrophage with an EC₅₀ of about 10 μM or less.

In a nineteenth embodiment, the invention provides a compound or pharmaceutically acceptable salts thereof, according to any of the foregoing inventions wherein said compound also inhibits TNF-alpha production resulting from LPS stimulation in macrophages with an EC₅₀ of about 20 μM or less.

In a twentieth embodiment, the invention provides a compound or pharmaceutically acceptable salts thereof, according to any of the foregoing inventions wherein said compound also inhibits TNF-alpha production resulting from LPS stimulation in macrophages in the presence of plasma with an EC₅₀ of about 100 μM or less.

In a twenty-first embodiment, the invention provides a compound or pharmaceutically acceptable salts thereof, according to any of the foregoing inventions wherein said compound also inhibits LPS induced TNF-alpha in a mouse with an ED₅₀ of about 100 mg/kg or less.

In a twenty-second embodiment, the invention provides a compound or pharmaceutically acceptable salts thereof, according to any of the foregoing inventions wherein said compound also inhibits collagen induced arthritis in a mouse with an ED₅₀ of about 500 mg/kg/day or less.

In a twenty-third embodiment, the invention provides a compound or pharmaceutically acceptable salts thereof, having an IC₅₀ of about 10 μM or less in a MK2 HTRF enzyme assay at 10 μM ATP and having a moiety of the formula 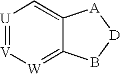
as a component of its complete structure, wherein
A is selected from the group consisting of N, S, O, bond, C═C, C and N;
B is selected from the group consisting of N, S, O, bond, C═C, C and N;
D is selected from the group consisting of C, N, S, O, and C═C;
wherein a double bond is optionally between A and D or B and D;
provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C or N;
V is C or N; and
W is C or N.

In a twenty-fourth embodiment the invention provides a compound of formula (I), 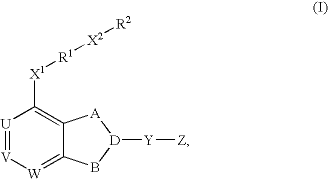
pharmaceutically acceptable salts thereof, metabolites thereof, isomers thereof, or pro-drugs thereof, wherein
A is selected from N, S, O, bond, C═C, C(J), C(J)₂ and N(J);
B is selected from N, S, O, bond, C═C, C(J), C(J)₂ and N(J);
D is selected from C, N, S, O, and C═C;
   wherein a double bond is optionally between A and D or B and D;
   provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C(J) or N;
V is C(J) or N;
W is C(J) or N;
   provided that U, V and W are not all N at the same time;
   J for each occurrence is independently H or halogen or is an optionally substituted moiety selected from Y-Z, —OR³, —S(R³), —S(O)R³, —S(O)₂R³, —N(R³)SO₂R³, —N(R³)C(O)N(R³)₂, —N(R³)₂, —N(R³)C(O)R³, —N(R³)-aliphatic —O—C(O)-aliphatic, —C(═O)—O-aliphatic-aryl, —C(═O)—O-aliphatic-cycloalkyl, —C(═O)—O-aliphatic-heterocyclyl, -phenyl-N(R³)-aliphatic-aryl, -phenyl-N(R³)-aliphatic-cycloalkyl, -phenyl-N(R³)-aliphatic-heterocyclyl, -phenyl-N(R³)-aliphatic, -phenyl-N(R³)-cycloalkyl, -phenyl-N(R³)-aryl, -phenyl-N(R³)—COOH, heterocyclyl-SO₂—NH-phenyl-, phenylalkoxy and CHO;
   J can be labeled at J¹, J² etc. to provide an unambiguous means of identifying which atom a moety is attached to;
      Y is selected from a bond, aliphatic, C(═O), C(═N(R³)), C(ζN—N(R³)₂), C(═N—OR³), S(O) and S(O₂), NR³—C(═O), C(═O)NR³, N(R³)C(═O)N(R³), and NR³, wherein each of the foregoing groups can optionally be preceded or followed by an optionally substituted aliphatic group; Z is a, H, halogen, CN, CF₃, N(R³)₂, OR³, 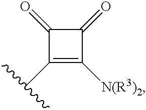
      or is independently an optionally substituted moiety selected from aliphatic, aryl, cycloalkyl, heterocyclyl, —(CH₂)ₙ—C(O)—N(R³)₂, —C(O)R³, —C(O)OR³, —C(O)N(R³)₂, —C(O)CF₃, —S(O)R³ and —SO₂R³; X¹ is a bond, halogen, N(R³), aliphatic, O, S, SO, SO₂, C(═NR³), C(═N—N(R³)₂), N(R³)SO₂, SO₂N(R³), N(R³)C(O)N(R³)S(O), N(R³)(CH₂)ₐN(R³)C(═O), N(R³)C(═O)N(R³), C(O)O, C(O), N(R³)C(O), C(O)N(R³), N(R³)C(O)N(R³), (CH₂)ₐN(R³), N(R³)(CH₂)ₐ, or (CH₂)ₐN(R³)(CH₂)ₐ; R¹ is a bond, a moiety of formula A, 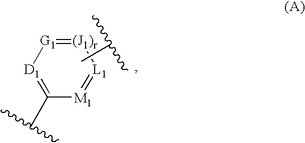
      or an an optionally substituted moiety selected from an aliphatic group, benzimidazolyl, benzofuranyl, benzoisothiazolyl, benzoisoxazolyl, benzoxazolyl, benzothiazolyl, benzothienyl, cycloalkyl, 2,3-dihydrobenzofuranyl, 1,1-dioxybenzoisothiazolyl, furanyl, 1H-imidazo[1,2-a]imidazolyl, imidazo [1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl, 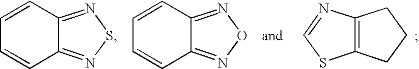
   wherein each of the foregoing groups can be optionally substituted by one or more R^{b};
   wherein when r is 1 then D₁, G₁, J₁, L₁ and M₁ are each independently selected from CR^{b} and N, provided that at least two of D₁, G₁, J₁, L₁ and M₁ are CR^{b}; or
   when r is 0, then one of D₁, G₁ , L₁ and M₁ is NR^{b}, one of D₁, G₁, L₁ and M₁ is CR^{b} and the remainder are independently selected from CR^{b}, S, O and N;
      when R¹ is not a bond then X² is a bond, aliphatic, N(R³), O, S, SO, SO₂, C(═NR³), C(═NR³), C)═N—N(R³)₂), N(R³)SO₂, SO₂N(R³), N(R³)C(O)N(R³)S(O), N(R³)(CH₂)ₐN(R³)C(═O), N(R³)C(═O)N(R³), C(O)O, C(O), N(R³)C(O), N(R³)C(O)N(R³), C(O)N(R³), (CH₂)ₐN(R³), N(R³)(CH₂)ₐ, or (CH₂)ₐN(R³)(CH₂)ₐ; or when R¹ is a bond then X² is a bond and R² is not a bond; R² is a bond, R³, a moiety of formula B, 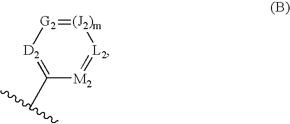
      or an an optionally substituted moiety selected from an aliphatic group, benzimidazolyl, benzofuranyl, benzoisothiazolyl, benzoisoxazolyl, benzoxazolyl, benzothiazolyl, benzothienyl, cycloalkyl, 2,3-dihydrobenzofuranyl, 1,1 -dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl, 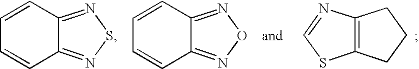
   wherein each of the foregoing groups can be optionally substituted by one or more R^{b};
   wherein when m is 1 then D₂, G₂, J₂, L₂ and M₂ are each independently selected from CR^{d} and N, provided that at least two of D₂, G₂, J², L₂ and M₂ are CR^{d}; or
   when m is O, then one of D₂, G₂, L₂ and M₂ is NR^{d}, one of D₂, G₂, L₂ and M₂ is CR^{d} and the remainder are independently selected from CR^{d}, S, O and N;
   R^{b} and R^{d} is an optionally substituted cycloalkyl or heterocyclyl ring fused with the ring to which it is attached;
   or R^{b} and R^{d} for each occurrence is independently hydrogen, a halogen, —OR³, NO₂, OCF₃, OH, CF₃, CN, C(O)H, C(O)OH, OCH₃ or is selected from an aliphatic, alkoxy, aliphatic-C(O)—, aliphatic-O(O)C—, aliphatic-S—, aliphatic-S(O)ₚ—, amido groups, amino, aminoalkoxy, aryl, arylalkoxy-, arylaliphatic-, aryloxy, aryl-C(O)—, aryl-O(O)C—, aryl-S—, aryl-S(O)ₚ—, aryl-aliphatic-S—, carboxamido, cycloalkyl, cycloalkyl-alkoxy, cycloalkyloxy, cycloalkyl-aliphatic-, cycloalkyl-C(O)—, cycloalkyl-O(O)C—, cycloalkyl-S—, cycloalkyl-aliphatic-S—, cycloalkyl-S(O)ₚ—, heterocyclyl, heterocycloalkoxy, heterocyclo-aliphatic, heterocyclyloxy, heterocyclyl-C(O)—, heterocyclyl-O(O)C—, heterocyclo-S—, heterocyclo-S(O)ₚ—, heterocyclo-aliphatic-S—, CF₃-carbonylamino, CF₃-sulfonamido, —Z¹—C(O)N(R³)₂, —Z¹—N(R³)—C(O)—R⁴, —Z¹—N(R³)—S(O)₂—R⁴, —Z¹—N(R³)—C(O)—N(R³)—R⁴, —N(R³)—C(O)R³, —N(R³)—C(O)OR³, —O—R⁴—C(O)-heterocyclyl-OR³, R^{e} and —CH₂OR^{e}, wherein each of the foregoing groups can be optionally substituted;
      p is 1 or 2;
      R^{e} for each occurrence is independently hydrogen, optionally substituted aliphatic, optionally substituted heterocyclyl, —(C₁-C₈)—NR^{f}R^{g}, Q-(CH₂)ₜ—NR^{f}R^{g}, -Q-(CH₂),—O-alkyl, -Q-(CH₂),—S-alkyl or -Q-(CH₂),—OH;
         R^{f} and R^{g} are each independently H, an aliphatic group, alkanoyl or SO₂-alkyl;
         or R^{f}, R^{g} and the nitrogen atom to which they are attached together form a five- or six-membered heterocyclic ring;
         t is an integer from 2 to 6;
         Q is a bond, O, S, S(O), S(O)₂, or NR^{h};
            R^{h} is H or an aliphatic group;
      Z¹ for each occurrence is independently a covalent bond or an aliphatic group;
         R³ for each occurrence is H, CN, CF₃, or is independently selected from the optionally substituted group aliphatic, cycloalkyl, aryl, heterocyclyl, —(CH₂)ₐ—C(O)—N(R⁴)₂, —OR⁴, —C(O)R⁴, —C(O)OR⁴, —C(O)N(R⁴)₂, —C(O)CF₃, —S(O)R⁵ and —SO₂R⁵;
   a is an integer from 1 to 5;

R⁴ for each occurrence is H or an optionally substituted moiety independently selected from aliphatic, aryl-aliphatic, cycloalkyl-aliphatic, heterocyclyl-aliphatic, cycloalkyl, heterocyclyl and aryl;
R⁵ is H or CF₃ or is an optionally substituted moiety selected from aliphatic, aryl and heterocyclyl;
   provided that: when U is CH; V is N; W is CH; B is S; A is CH; D is C; X¹ is O, S(O)ₙ, C═CH₂, CH—CH, CH(OH), C(═O), C(═O)NH, OCH₂, CH₂, or C(OH)(CH₂OH), wherein n is 0, 1 or 2; then R¹—X²—R² is not phenyl, cycloalkyl, pyridinyl, furanyl or 1,3,4-thiadiazolyl;
each of which can be optionally substituted by one or more halogen, optionally substituted alkyl, optionally substituted alkenyl, COOR, NHC(═O)R, C(═O)NHR, COR, NH2, CN, 1-pyrazolyl or alkoxy;
wherein R is H or is selected from alkyl, alkylaryl and morpholinyl, wherein each of the foregoing groups can be optionally substituted;
   when U is CH; V is N; W is CH; B is S; A is CH; D is C; then X¹—R¹—X²—R² is not H, Cl, Br, or —SCH₂C(═O)NH₂; when U is CH; V is N; W is CH; B is S; A is CH; D is C; then U is not C(J) wherein J is 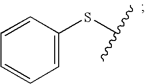
   when U is CH; V is N; W is CH; B is S; A is CH; D is C; X¹ is O, S(O)ₙ, C═O, or NCH₃, wherein n is 0, 1 or 2; or when U is CH; V is N; W is CH; B is S; A is CH; D is C; X¹—R¹—X²—R² is morpholinyl; then Y-Z is not
—C(═O)—NH—CH₃,
—C(═O)—N(CH₃)₂,
—C(═O)—NH—(CH₂)₂OH,
—C(═O)—NH—CH(CH₃)—C(═O)—OH,
—C(═O)—NH—CH₂OH,
—C(═O)—NH—CH₂—C(═O)—OCH₃,
—C(═O)—NH—CH₂—C(═O)—NH₂,
—C(═O)═NH—CH₂—CHO,
—C(═O)—CH(CH₃)—C(═O)—NHCH₃,
—C(═O)—CH₂—CN,
—CH═CH—C(═O)—NH₂,
—CH(OH)—CH(OH)—C(═O)—NHCH₃,
—C═N(CH₃)NH₂,
—C═N(H)—OCH₃,
—O-phenyl wherein the phenyl is substituted by —CH₂═CH₂—C(═O)—OH, —CH₂OH or —NH—C(═O)—O—C(CH₃)₃,
—C(═O)-phenyl-morpholinyl,
oxadiazolyl optionally substituted by NH₂, S, CH₃, —NH—CH₃, or phenyl, triazolyl optionally substituted by CH₃ or CH₃ and NH₂, isoxazolyl substituted with NH₂, 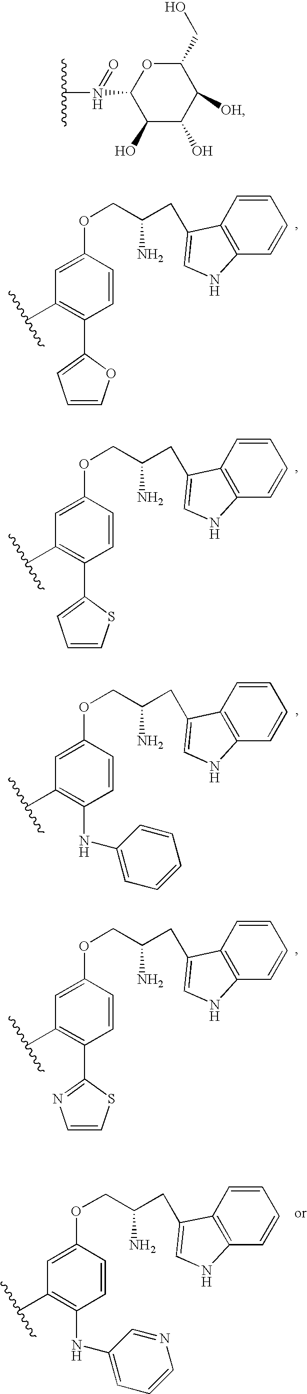 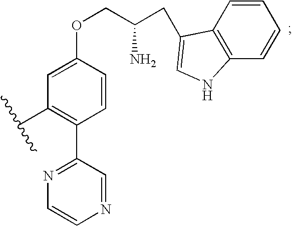
   when U is CH, V is N, W is CH, B is S, A is CH, D is C, and X¹ is NH then Y-Z is not —C(═O)—NH—CH₃ or 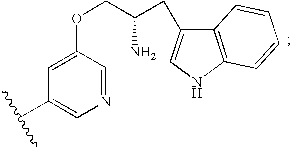
   when U is CH; V is N; W is CH; B is S; A is CH; D is C; Y-Z is —C(═O)NH₂ then X¹—R¹—X²—R² is not 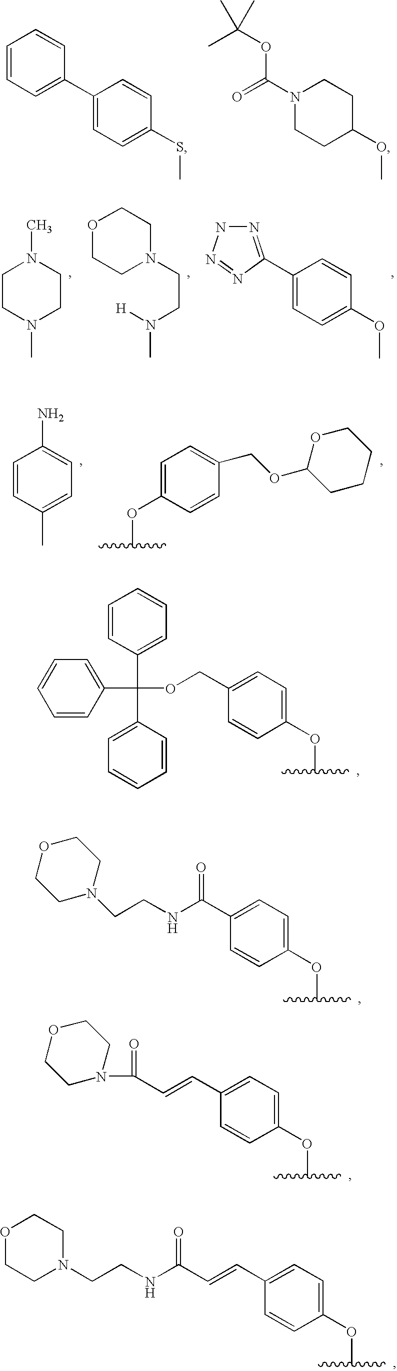 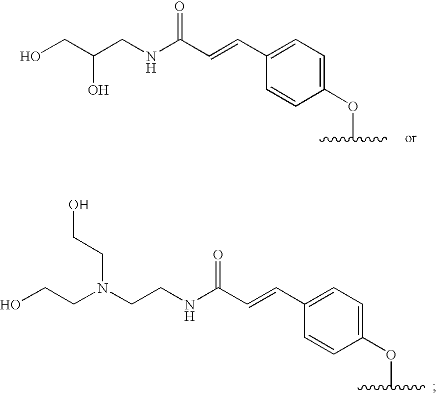
   when U is CH; V is N; W is CH; B is S; A is CH; D is C; then X¹—R¹—X²—R² is not phenyl optionally substituted with one or more CF₃, Cl or F; and when A is C(J); D is C; and there is a double bond between A and D; B is S; U is N; V is C; W is C; Y-Z is H, methyl, ethyl or propyl; X¹—R¹—X²—R² is —NH₂; then J is not a substituted phenyl; when Formula (I) is 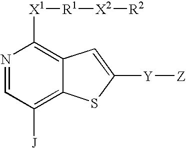
   wherein
J is NH₂ or NHCH₃;
Y is a bond;
Z is selected from phenyl, 4,5,6,7-tetrahydrobenzo[b]thienyl, 1,3-dihydrobenzo[c]isothiazolyl, cyclohexyl, cyclopentyl, ethyl, imidazolyl, methyl, furanyl, pyrazolyl, pyridinyl, pyrrolidinyl, pyrrolyl, tetrahydrofuranyl, thiazolyl, thienyl, and thiomorpholine 1,1-dioxide, any of which can be optionally substituted or Z is —CH═CH—CH₃;
then X¹—R¹—X²—R² is not —C(═O)NH₂;
   when Formula (I) is 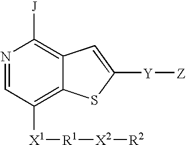
   wherein
J is —C(═O)NH₂;
Y is a bond;
Z is cyclohexyl, thiazolyl oroptionally substituted phenyl;
then X¹—R¹'X²—R² is not NH₂ or NHCH₃;Y is a bond;
Z is selected from phenyl, 4,5,6,7-tetrahydrobenzo[b]thienyl, 1,3-dihydrobenzo[c]isothiazolyl, cyclohexyl, cyclopentyl, ethyl, imidazolyl, methyl, furanyl, phenyl, pyrazolyl, pyridinyl, pyrrolidinyl, pyrrolyl, tetrahydrofuranyl, thiazolyl, thienyl, and thiomorpholine 1,1-dioxide, any of which can be optionally substituted or Z is —CH═CH—CH₃; then J is not —C(═O)NH₂;
   a compound of Formula (I) is not 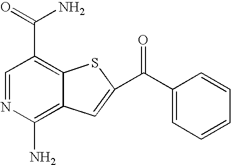
   a compound of Formula (I) is not 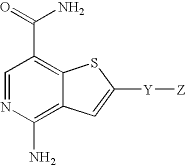
   wherein Y is ethyl or propyl and Z is phenyl or OCH₃; a compound of Formula (I) is not 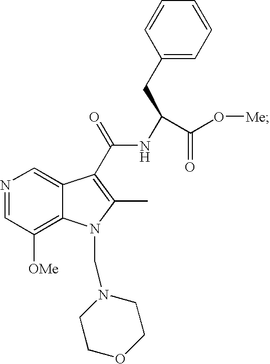
   a compound of Formula (I) is not 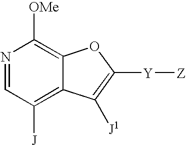
   wherein
J is —C(═O)—NH—R wherein R is selected from phenyl, pyrazolyl, pyridyl, isoxazolyl and pyridinyl and can be optionally substituted;
J¹ is H, pyridinyl or tetrahydrofuranyl;
Y is —C(═O) or a bond; and
Z is methyl, ethyl, tetrahydropyranyl, OCH₃ or optionally substituted piperidinyl;
then X¹—R¹—X²—R² is not OCH₃;
a compound of Formula (I) is not 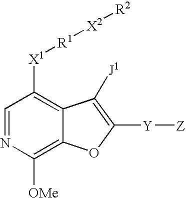
   wherein
X¹—R¹—X²—R² is —C(═O)—NH—R³ wherein R³ is selected from phenyl, pyrazolyl, pyridyl, isoxazolyl and pyridinyl and can be optionally substituted;
Y is—C(═O) or a bond;
Z is methyl, ethyl, tetrahydropyranyl, OCH₃ or optionally substituted piperidinyl; and J¹ is H, pyridinyl or tetrahydropyranyl;
a compound of Formula (I) is not 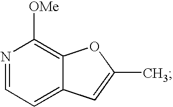
   a compound of Formula (I) is not 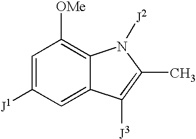
   wherein J¹ is Cl, F or H;
J² is —CH₂-phenyl wherein the phenyl is optionally substituted, —CH₂CH₂CH₂-piperazinyl wherein the piperazinyl is optionally substituted, —CH₂—CH₂-morpholinyl or CH₂—CH₂—CH₂-morpholinyl;
J³ is optionally substituted and is selected from —C(═O)—NH—cyclopentyl, —C(═O)—NH—cyclohexyl, —C(═O)—NH—CH₂CH₃, —C(═O)—NH—1,3,3-trimethylbicyclo[2.2.1] heptan-2-yl, —C(═O)—NH—CH(—CH₂-phenyl)—CO₂CH₃, —C(═O)—NH—CH(CO₂CH₃)—CH₂-phenyl, —C(═O)—NH—CH₃, —C(═O)—NH-phenyl, —C(═O)-tetrahydroquinolinyl, —C(═O)—NH—CH(CH₃)—CH₂—CH₃, —C(═O)—NH—CH(—CH₂-phenyl)—CO₂CH₃, —C(═O)—NH—CH(—CH₂-phenyl)-oxazolyl; —C(═O)—NH—CH(—CH₂-phenyl)—C(═O)—NH₂, —C(═O)—NH—CH(—CH₂-phenyl)—C(═O)—N(CH₃)(OCH₃), —C(═O)—NH—CH(—CH₂-phenyl)-[1,2,4]oxadiazolyl, —C(═O)—NH—CH(—CH₂—CH₂—S—CH₃)—C(═O)—OCH₃, —C(═O)—NH—CH(CH(CH₃)₂)—C(═O)—OCH₃, —C(═O)═NH—CH(—CH₂-phenyl)—C(═O)—OC(CH₃)₃, —C(═O)—NH—CH—(CH₂-phenyl)—C(═O)—OCH₂CH₃, —C(═O)—NH—CH(CH₃)-phenyl, —C(═O)—NH—CH(—CH₂-thienyl)—C(═O)—OCH₃, —C(═O)═NH—CH(thiazolyl)—C(═O)—OCH₃, and —C(═O)—NH—CH(—CH₂-phenyl)-tetrazolyl;
   a compound of Formula (I) is not 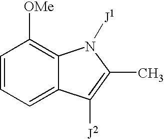
   wherein
J¹ is selected from H, pentyl, —CH₂—CH₂-piperidinyl, —CH₂—CH₂—OCH₃, —CH₂-pyridinyl, —CH₂—CH₂—CH₂-morpholinyl, —CH₂—CH₂—N(CH₃)₂, —CH₂—CH₂-pyrrolidinyl, —N(CH₂CH₃)₂, —CH₂—CH₂-cyclohexyl, —CH₂—CH₂—N(CH(CH₃)₂), —CH₂—CH₂—OCH₂CH₃, —CH₂—CH₂—CH₂—OCH₂-phenyl, —CH₂-tetrahydrofuranyl, —CH₂—CH₂-morpholinyl wherein the morpholinyl is optionally substituted, —CH₂—CH₂—O-phenyl, —C(═O)—O—C(CH₃)₃, and COOH; and
J² is —C(═O)—NH—1,3,3-trimethylbicyclo[2.2. 1]heptan-2-yl or —CH₂—CH₂-morpholinyl;
   a compound of Formula (I) is not 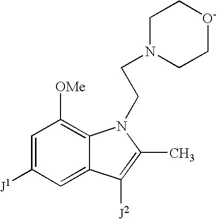
   wherein
J¹ is OCH₃ or CH₃;
J² is —C(═O)—NH—1,3,3-trimethylbicyclo[2.2.1]heptan-2-yl or —C(═O)—NH—CH(CO₂CH₃)—CH₂—phenyl;
   a compound of Formula (I) is not 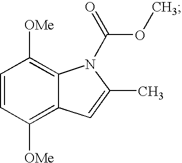
   when Formula (I) is 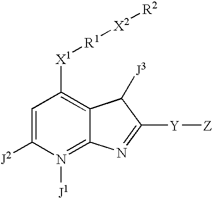
   wherein
J¹ is H, OH or —CH₂—CH₂-morpholinyl;
J² is H or —S—CH₂—CH₂—CH₃;
J³ is —C(═O)—NH—CH(—CH₂-phenyl)—CO₂CH₃ or —C(═O)—C(═O)-piperazinyl wherein the piperazinyl is optionally substituted;
then X¹—R¹—X²—R² is not OCH₃;
   when formula (I) is 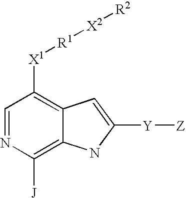
   wherein
J is optionally substituted and is selected from 1,2,4-triazolyl, pyrazolyl and pyrazinyl;
Y is a bond;
Z is H or CH₃;
then X¹—R¹—X²—R² is not OCH₃;
   when Formula (I) is 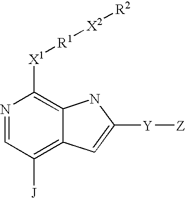
   wherein
Y is a bond;
Z is H or CH₃,
X¹—R¹—X²—R² is optionally substituted and is selected from 1,2,4-triazolyl, pyrazolyl or pyrazinyl;
then J is not OCH₃;
   a compound of Formula (I) is not 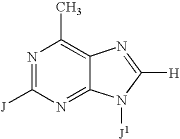
   wherein
J is H or CH₃ and J¹ is optionally substituted tetrahydrofuranyl;
   a compound of Formula (I) is not 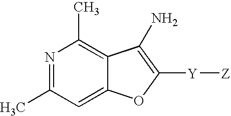
   wherein
Y is —C(═O) and Z is optionally substituted phenyl;
   a compound of Formula (I) is not 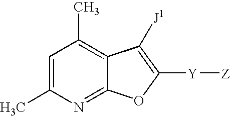
   wherein
J¹ is —NH—C(═O)—NH₂, NH2 or pyridinyl; Y is —C(═O); and
Z is optionally substituted thienyl or optionally substituted phenyl;
   a compound of Formula (I) is not 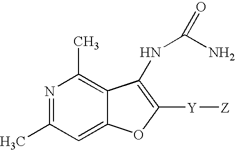
   wherein
Y is —C(═O) and Z is phenyl substituted with two methyls;
   when Formula (I) is 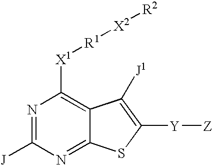
   wherein
J is selected from H, —NH—C(═O)═NH═CH(C(C═O)OH)—CH₂—CH₂)CH₃)₂, CH₃, isopropyl, —NH—CH₂—CH₃ and —NH'CH₂—CH₂OH;
J¹ is selected from cyclohexyl, cyclopentyl, pyridinyl and optionally substituted phenyl;
Y is a bond;
Z is selected from pyridinyl, pyridazinyl, pyrimidinyl, cyclohexyl, cyclopentyl and optionally substituted phenyl;
then X¹—R¹—X²—R² is not —NH-ethyl wherein the ethyl is optionally substituted with OH;
   a compound of Formula (I) is not 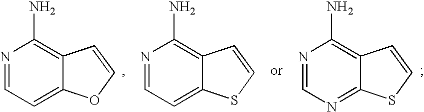
   a compound of Formula (I) is not 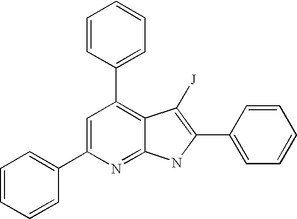
   wherein J is —C(═O)—C(═O)-piperazinyl wherein the piperazinyl is substituted; a compound of Formula (I) is not 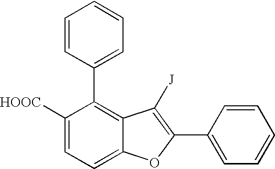
   wherein J is H or —C(═O)—OCH₃; a compound of Formula (I) is not 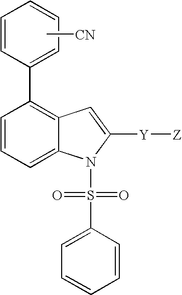
   wherein Y is —C(═O) and Z is substituted phenyl; a compound for Formula (I) is not 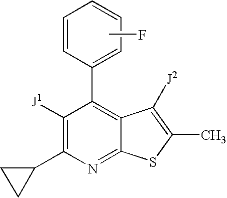
   wherein
J¹ is selected from 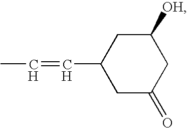
   —C(═O)—OCH₃, —CH₂OH, CHO, —CH═CH—CHO, —CH═CH—CH(OH)—CH₂—CH(OH)—CH₂—CO₂CH₂CH₃, —CH═CH—CH(OH)—CH₂—CH(OH)—CO₂Na, —CH═CH—CH(OH)—CH₂—CH(OH)—CO₂Ca, —CH═CH—CH(OH)—CH₂—CH(OH)—CH₂CO₂H, substituted 2,2-dimethyl-1,3-dioxane and —CH═CH—CH₂—CH(OH)—CH₂—C(═O)—CH₂—CO2₂CH₂CH₃;
J³ is selected from ethyl, substituted benzyl, —CH₂—CH₂—CH(phenyl)-CH₃ and substituted phenyl;
   when Formula (I) is 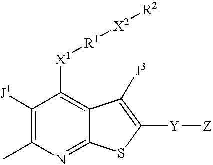
   wherein
J¹ is selected from —CH═CH—CH(OH)—CH₂—CH(OH)—CH₂—CO₂CH₂CH₃, 4-hydroxytetrahydropyran-2-one, optionally substituted 2,2-dimethyl-1,3dioxane and —CH═CH—CH(OH)—CH₂—CH(OH)—CH₂CO₂Ca;
J² is selected from —C(CH)═CH₂, cyclopropyl and cyclohexyl;
J³ is selected from —CH═CH—CH₃, n-hexyl, butoxy, 2-pyrimidinyl, 2-thienyl, 2-furanyl, piperazinyl, optionally substituted phenyl, optionally substituted phenoxy and optionally substituted benzyl;
Y is a bond and Z is H,
then X¹—R¹—X²—R² is not phenyl substituted with F or phenyl substituted with F and CH₃;
   when Formula (I) is 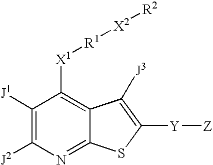
   wherein
J¹ is selected from —CH═CH—CH(OH)—CH₂—CH(OH)—CO₂CH₂CH₃, —CH=CH—CH(OH)—CH₂—CH(OH)—CH₂—CO₂H, —CH═CH—CH(OH)—CH₂—CH(OH)—CH₂—CO₂Ca, 4-hydroxy-tetrahydropyran-2-one, substituted 2,2-dimethyl-1,3-dioxane, —CH═CH—CH(OH)—CH₂—C(═O)—CH₂CO₂CH₂CH₃, —CH═CH—CH(OH)—CH₂—C(═O)—CH₂—CH₂—CO₂CH₂CH₃, —CH═CH—C(═O)═CH₂—C(═O)—CH₂—CO₂CH₂CH₃, and —CH═CH—C(═O)—CH(OH)—CH₂—CO₂CH₂CH₃;
J² is selected from H, isopropyl, methyl, n-propyl, n-hexyl, —C(CH₃)═CH₂ and cyclopropyl;
J³ is selected from H, isopropyl, phenyl, n-propyl, Cl, OCH₃, N(CH₃)₂, benzyl, butyl, ethyl, methyl, isobutyl and cyclopentylmethyl;
Y is a bond; and
Z is selected from methyl, isopropyl, n-propyl, ethyl, n-butyl, Br, Cl, hexyl, —CH═CH₂, phenyl, 2-naphthyl and 3-pyridyl;
then X¹—R¹—X²—R² is not phenyl substituted with F, Cl or CH₃ or phenyl substituted with F and CH₃;
   when Formula (I) is 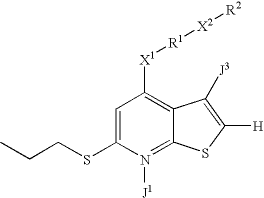
   wherein J¹ is OH or H; and
J² is —C(═O)-piperazinyl wherein the piperazinyl is substituted with CH₃ and phenylcarbonyl;
then X¹—R¹—X²—R² is not —OCH₃—CF₃ or OH;
   a compound of Formula (I) is not 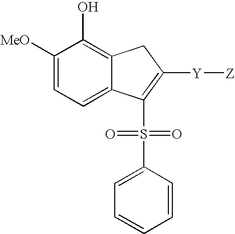
   wherein Y is CH₂, —CH(OH) or —C(═O);
Z is isoquinolinyl or Z is phenyl optionally substituted with OH;
   a compound of Formula (I) is not 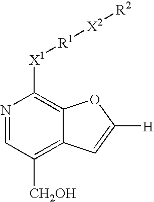
   wherein X¹—R¹—X²—R² is —NH-thiazolyl wherein the thiazolyl is optionally substituted with CN; a compound of Formula (I) is not 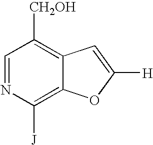
   wherein J is is —NH-thiazolyl wherein the thiazolyl is optionally substituted with CN; a compound of Formula (I) is not 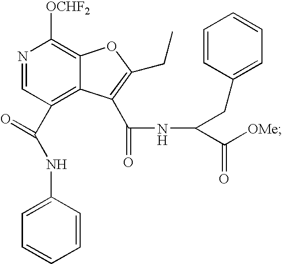
   a compound of Formula (I) is not 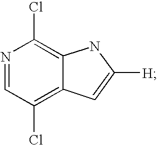
   a compound of Formula (I) is not 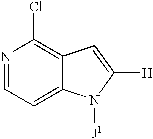
   wherein J¹ is H or —C(═O)—N(CH₃)₂; a compound of Formula (I) is not 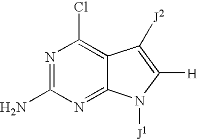
   wherein
J¹ is substituted tetrahydrofuranyl and J² is CN, ethyl, CH₃ or H;
   a compound of Formula (I) is not 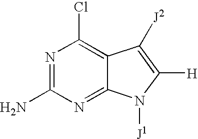
   wherein
J¹ is substituted tetrahydrofuranyl and j² is CN, ethyl, methyl or H;
   a compound of Formula (I) is not 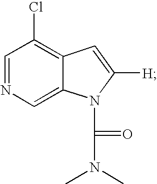
   when Formula (I) is 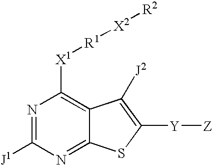
   wherein
J¹ is H or CH3;
J² is phenyl substituted with F;
Y is a bond; and Z is pyridazinyl, pyrimidinyl or pyridinyl;
then X¹—R¹—X²—R² is not Cl;
   a compound of Formula (I) is not 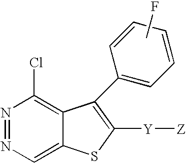
   wherein Y is a bond and Z is pyridinyl;
   a compound of Formula (I) is not 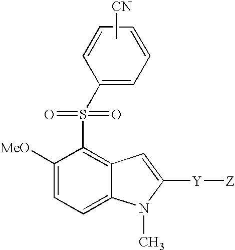
   wherein
Y is —C(═O) and Z is optionally substituted phenyl;
   when Formula (I) is 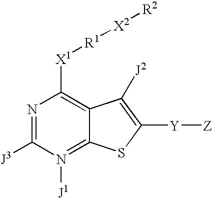
   wherein
J¹ is H or OH;
j² is phenyl substituted with F, optionally substituted tetrahydrofuranyl or —C(═O)-piperazinyl wherein the piperazinyl is optionally substituted;
J³ is H or —S-propyl;
Y is a bond; and
Z is pyridinyl, NH₂ or H;
then X¹—R¹—X²—R² is not —NH—CH₂—C(═O)—OCH₂CH₃, —NH—CH₂CH₃, —NH—CH₂-benzo[1,3]dioxazolyl, —NH-benzo[1,3]dioxazolyl, —NH—CH₂-phenyl, —NH—CH₂—CH(CH₂CH₃)₂, —NH—CH₂CH₂—OEt wherein the Et is substituted with OH, —NH—CH₂—C(═O)—NH—CH(CH₂—CH(CH₃)₂)—COOH, —NH—C(═O)—OCH₂CH₃, —NH—CH₂—C(═O)OH or —NH—CH₂CH₂—OCH₂—CH₂—CH₂OH;
   when Formula (I) is 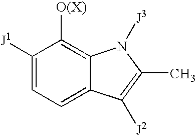
   wherein
J¹ is H or OCH₃,
J² is H, —C(═O)—CH(—CH₂-phenyl)—C(═O)—OCH₃ or —C(═O)—NH—C(—CH₂-phenyl)H—C(═O)—
OCH₃; and
J³ is H or —CH₂—CH₂-morpholinyl;
then X is not butyl, pentyl or phenyl;
   a compound of Formula (I) is not 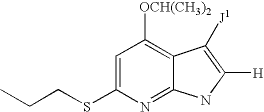
   wherein
J¹ is not —C(═O)-piperazinyl wherein the piperazinyl is optionally substituted;
   when Formula (I) is 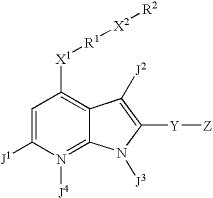
   wherein
J¹ is —CH═CH₂ or —S-propyl;
J² is —C(═O)-piperazinyl wherein the piperazinyl is optionally substituted;
J³ is H or —SO₂-phenyl;
J⁴ is H or OH;
Y is a bond; and
Z is optionally substituted phenyl;
then X¹—R¹—X²—R² is not —CH═CH₂ or —S-propyl;
   a compound of Formula (I) is not

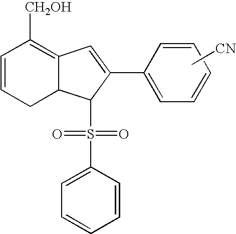

In a twenty-fifth embodiment the invention provides a compound or pharmaceutically acceptable salts thereof, metabolites thereof, isomers thereof, or pro-drugs thereof, according to any of the foregoing inventions wherein, B is S, N or O; X¹ is a bond, O, S or NH.

In a twenty-sixth embodiment the invention provides compound or pharmaceutically acceptable salts thereof, metabolites thereof, isomers thereof, or pro-drugs thereof, according to any of the foregoing inventions wherein,
U is CH; V is N; W is CH or CNH₂; A is CH; D is C and there is a double bond between A and D; B is S or N;
Y-Z is tetrazole, —C(═O)N(R³)₂, —C(═O)NR³OR³, —NR³C(═O)R³ or —C(═O)OR³;
X¹ is a bond, O or NH; and
R¹ is an optionally substituted group selected from phenyl, benzimidazolyl, benzofuranyl, benzoisothiazolyl, benzoisoxazolyl, benzoxazolyl, benzothiazolyl, benzothienyl, 2,3-dihydrobenzofuranyl, 1,1 -dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl,

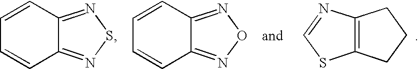

In a twenty-seventh embodiment the invention provides a compound or pharmaceutically acceptable salts thereof, metabolites thereof, isomers thereof, or pro-drugs thereof, according to any of the foregoing inventions wherein,
R¹ is phenyl or piperidinyl, both of which can be optionally substituted with R^{b}.

In a twenty-eighth embodiment the invention provides a compound or pharmaceutically acceptable salts thereof, metabolites thereof, isomers thereof, or pro-drugs thereof, according to any of the foregoing inventions wherein
Y-Z is a tetrazole, —C(═O)N(R³)₂, —C(═O)NR³OR³ or —C(═O)OR³.

In a twenty-ninth embodiment the invention provides a compound or pharmaceutically acceptable salts thereof, metabolites thereof, isomers thereof, or pro-drugs thereof, according to any of the foregoing inventions wherein
X¹ is NH or a bond;
B is S.

In a thirtieth embodiment the invention provides a compound or pharmaceutically acceptable salts thereof, metabolites thereof, isomers thereof, or pro-drugs thereof, according to any of the foregoing inventions wherein
Y-Z is —C(═O)N(H)₂; and
X² is a bond, NH or CH₂ and R² is unsubstituted benzoxazolyl or phenyl optionally substituted with OH, CN, CONH₂ or Br.

In a thirty-first embodiment the invention provides a compound according to any of the foregoing inventions wherein the compound is 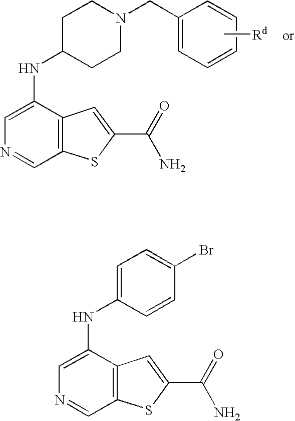
wherein R^{d} is selected from OH, CN, H and CONH₂.

In a thirty-second embodiment, the invention provides for a compound of formula (I) 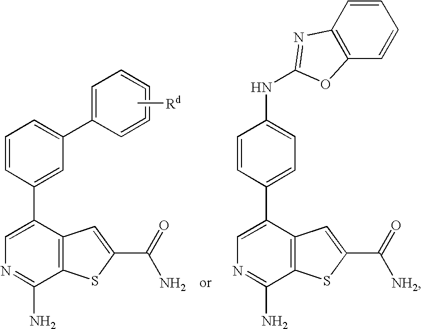
wherein the compound is wherein R^{d} is selected from OH, CN, H and CONH₂.

In a thirty-third embodiment, the invention provides for a compound of formula (I) wherein the compound is

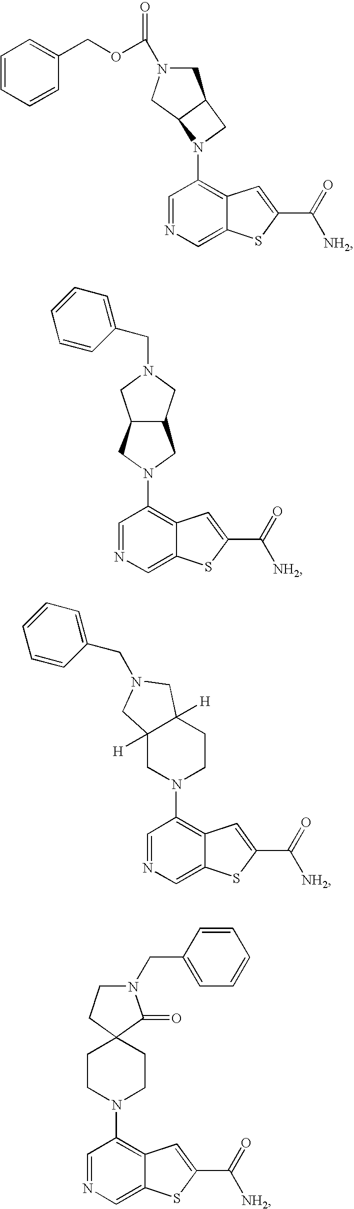

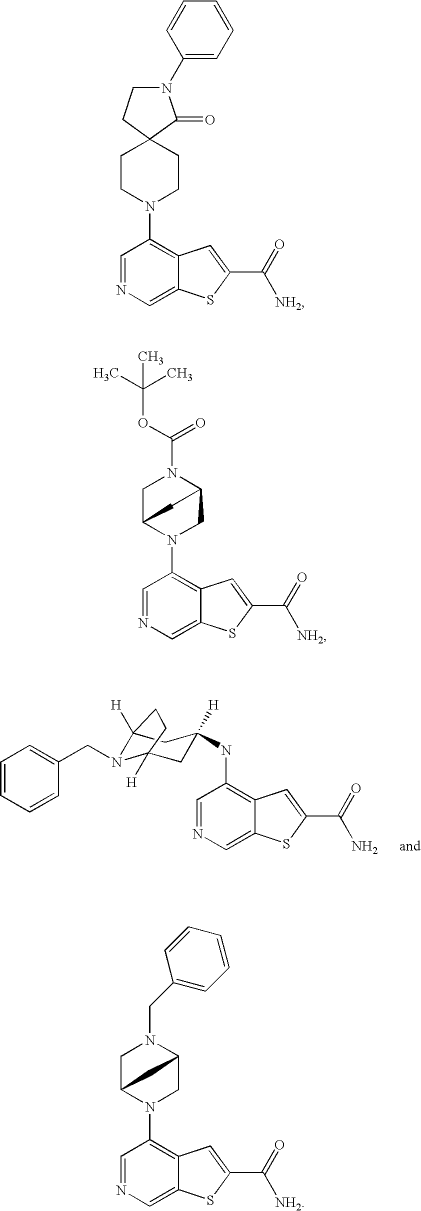

In a thirty-fourth embodiment the invention provides a method of inhibiting one or more protein kinase activity in a patient comprising administering a therapeutically effective amount of a compound of formula (I) or a physiologically acceptable salt, prodrug or biologically active metabolites thereof to said patient.

In a thirty-fifth embodiment the invention provides a method for inhibiting COT in a human subject suffering from a disorder in which COT activity is detrimental, comprising administering a therapeutically effective amount of a compound of formula (I) or a physiologically acceptable salt, prodrug or biologically active metabolites thereof to said patient.

In thirty-sixth embodiment the invention provides a method for inhibiting MK2 in a human subject suffering from a disorder in which MK2 activity is detrimental, comprising administering a therapeutically effective amount of a compound of formula (I) or a physiologically acceptable salt, prodrug or biologically active metabolites thereof to said patient.

In a thirty-seventh embodiment the invention provides a method of treating a condition in a patient comprising administering a therapeutically effective amount of a compound of formula (I) or a physiologically acceptable salt, prodrug or biologically active metabolites thereof to said patient, wherein said condition is selected from the group comprising rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-I autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, and cancers such as lung, breast, stomach, bladder, colon, pancreas, ovarian, prostate and rectal cancer and hematopoietic malignancies (leukemia and lymphoma), and hematopoietic malignancies (leukemia and lymphoma), and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

In thirty-eighth embodiment the invention provides a pharmaceutical composition comprising a compound according to formula (I) and a pharmaceutically acceptable carrier or diluent.

### DETAILED DESCRIPTION OF THE INVENTION

Protein Kinases

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and S/T kinases as a result of their substrate specificity.

Serine/Threonine Kinases

S/T kinases are a large sub-family of protein kinases that specifically transfer a phosphate group to a terminal hydroxyl moiety of specific serine or threonine residues (Hanks et al., (1988) Science, 241: 42-52). A number of S/T kinase family members are involved in inflammatory signaling, tumor growth or cellular transformation. For example, the mitogen-activated protein kinases (MAPKs) are S/T kinases that act as intermediates within the signaling cascades of Toll like receptors (TLRs), such as TLR4, growth/survival factors, such as EGF, and death receptors, such as the TNF receptor. Activation of MAPKs, such as extracellular signal-regulated kinases (ERK1-2), p38α, c-Jun N-terminal kinase (JNK) or MAPKAP-K2 (MK2) have been shown to transduce signaling in cells, such as macrophages, resulting in the extracellular production of pro-inflammatory cytokines, such as TNF.

TPL-2 is a S/T kinase which is homologous to MAP kinase kinase kinases (MAP3K) in its catalytic domain (Salmeron, et al., (1996) EMBO J., 15, 817-826) and is >90% identical to the proto-oncogene product of human COT (Aoki et al., (1993) J. Biol. Chem., 268, 22723-22732). TPL-2 was originally identified, in a C-terminally deleted form, as the product of an oncogene associated with Moloney murine leukemia virus-induced T cell lymphomas in rats (Patriotis, et al., (1993) Proc. Natl. Acad. Sci. USA 90, 2251-2255). TPL-2 is also highly homologous to the kinase NIK, which has been shown to regulate the inducible degradation of I□B-α (Malinin et al., (1997) Nature, 385, 540-544; WO 97/37016; May and Ghosh, (1998) Immunol. Today, 19, 80-88). TPL-2 is essential for the activation of a MAP2K (MEK1-2) and subsequently MAPK (extracellular signal-regulated kinase, ERK1-2) in macrophages stimulated by TLR agonists, such as lipopolysachharide (LPS). TPL-2 plays a crucial role in the regulation of LPS-induced TNF, IL-1β and COX-2 induced prostaglandin-E2 production in macrophages (Tsichlis et al, (2000), Cell, 103, 1071; Tsichlis et al, (2002), EMBO J, 21, 4831-4840). The expression of COT/TPL-2 in various tumors (Tsanisi et al., (2000), Int J Mol Med, 5, 583) and the defect in TNF production observed in COT knockout mice (Tsichlis et al, (2000), Cell, 103, 1071) suggests that inhibition of COT may be a useful approach in the treatment of cancer, inflammation or other diseases mediated by pro-inflammatory cytokines.

MK2 (MAPKAP-K2) is an S/T kinase critically involved in inflammatory processes. MK2 is a substrate for the p38 MAP Kinase pathway (Stokoe et al., (1992), EMBO J., 11, 3985-3994; Ben-Levy et al., (1995), EMBO J., 14, 5920-5930). Activation of MK2 in immune cells results in an array of cellular responses including cytokine production, proliferation and activation. Knockout mice defective in MK2 production are healthy and fertile but fail to produce cytokines such as tumor necrosis factor (TNF) in response to inflammatory stimuli (Kotlyarov et al., (1999), Nat. Cell Biol, 1, 94-97.). MK2 may alter gene expression by phosphorylation of mRNA-binding proteins (Winzen et al., (1999), EMBO J., 18, 4969-4980; Lasa et al., (2000), Mol. Cell. Biol., 20, 4265-4274; Rousseau et al., (2002), EMBO J., 21, 6505-6514; Bollig et al., (2003), Biochem. Biophys. Res. Commun, 301, 665-670; Tran et al., (2003), Mol. Cell. Biol., 23, 7177-7188.), transcription factors (Heidenreich et al., (1999), J. Biol. Chem., 274, 14434-14443) or other proteins (Stokoe et al,. (1992), FEBS Lett., 313, 307-313; Sutherland et al., (1993), Eur. J. Biochem., 217, 715-722; Werz et al, (2000), Proc. Natl. Acad. Sci._{—}USA, 97, 5261-5266). The defect in TNF production in MK2 knockouts suggests that the antiinflammatory effect of p38 MAPK inhibitors may be largely due to blockade of activation of MK2. Inhibitors of MK2 may be effective treatments of inflammation or other diseases mediated by pro-inflammatory cytokines.

Protein Tyrosine Kinases.

Protein tyrosine kinases (PTKs) are enzymes that catalyse the phosphorylation of specific tyrosine residues in cellular proteins. This post-translational modification of these substrate proteins, often enzymes themselves, acts as a molecular switch regulating cell proliferation, activation or differentiation (for review, see Schlessinger and Ulrich, 1992, Neuron 9:383-391). Aberrant or excessive PTK activity has been observed in many disease states including benign and malignant proliferative disorders as well as diseases resulting from inappropriate activation of the immune system (e.g. autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, and infantile hemangiomas).

Tyrosine kinases can be of the receptor-type (having extracellular, transmembrane and intracellular domains) or the non-receptor type (being wholly intracellular).

Receptor Tyrosine Kinases (RTKs). The RTKs comprise a large family of transmembrane receptors with diverse biological activities. At present, at least nineteen (19) distinct RTK subfamilies have been identified. The receptor tyrosine kinase (RTK) family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response (e.g., cell division, differentiation, metabolic effects, and changes in the extracellular microenvironment; see Schlessinger and Ullrich, 1992, Neuron 9:1-20).

Non-Receptor Tyrosine Kinases. Non-receptor tyrosine kinases represent a collection of cellular enzymes which lack extracellular and transmembrane sequences. Over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. The Src subfamily of non-receptor tyrosine kinases is comprised of the largest number of PTKs and include Src, Yes, Fyn, Lyn, Lck, Blk, Hck, Fgr and Yrk. The Src subfamily of enzymes has been linked to oncogenesis and immune responses. A more detailed discussion of non-receptor tyrosine kinases is provided in Bohlen, 1993, Oncogene 8:2025-2031, which is incorporated herein by reference.

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

In a related aspect the invention provides a method for inhibiting COT in a human subject suffering from a disorder in which COT activity is detrimental, comprising administering to the human subject a compound of Formula (1) such that COT activity in the human subject is inhibited and treatment is achieved.

In another related aspect the invention provides a method for inhibiting MK2 in a human subject suffering from a disorder in which MK2 activity is detrimental, comprising administering to the human subject a compound of Formula (I) such that MK2 activity in the human subject is inhibited and treatment is achieved.

A compound of formula (I) or a salt thereof or pharmaceutical compositions containing a therapeutically effective amount thereof is useful in the treatment of a disorder selected from the group comprising rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-I autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, and cancers such as lung, breast, stomach, bladder, colon, pancreas, ovarian, prostate and rectal cancer and hematopoietic malignancies (leukemia and lymphoma), and hematopoietic malignancies (leukemia and lymphoma), and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of formula (I) of the invention can be used alone or in combination with another therapeutic agent to treat such diseases. It should be understood that the compounds of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the anti-IL-18 antibodies of this invention. Non-limiting examples of therapeutic agents for rheumatoid arthritis with which a compound of formula (I) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (HUMIRA™), (PCT Publication No. WO 97/29131), CA2 (REMICADE™), CDP 571, and soluble p55 or p75 TNF receptors, derivatives, thereof, (p75TNFR1gG (ENBREL™) or p55TNFR1gG (Lenercept), and also TNFα converting enzyme (TACE) inhibitors; similarly IL-1 inhibitors (Interleukin-1-converting enzyme inhibitors, IL-1 RA etc.) may be effective for the same reason. Other preferred combinations include Interleukin 11. Yet another preferred combination are other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of formula (I) of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofebil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL- 1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFgα or IL-1 (e.g. IRAK, NIK, IKK, p38 or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ).Preferred examples of therapeutic agents for Crohn's disease in which a compound of formula (I) can be combined include the following: TNF antagonists, for example, anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of formula (I) can be combined with corticosteroids, for example, budenoside and dexamethasone; sulfasalazine, 5-aninosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone hcl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam hcl; cyanocobalamin; folic acid; levofloxacin; methylprednisolone; natalizumab and interferon-gamma.

Non-limiting examples of therapeutic agents for multiple sclerosis with which a compound of formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-1 6, EMAP-II, GM-CSF, FGF, and PDGF. A compound of formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of formula (I) may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of formula (I) may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP- 1258); sTNF-R1, talampanel, teriflunomide,TGF-beta2, tiplimotide, VLA-4 antagonists (for example, TR-14035, VLA4 Ultrahaler, Antegran-ELAN/Biogen), interferon gamma antagonists, IL-4 agonists.

Non-limiting examples of therapeutic agents for Angina with which a compound of formula (I) of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil hcl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril, bisoprolol fumarate.

Non-limiting examples of therapeutic agents for Ankylosing Spondylitis with which a compound of formula (I) can be combined include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, infliximab.

Non-limiting examples of therapeutic agents for Asthma with which a compound of formula (I) can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, metaproterenol sulfate.

Non-limiting examples of therapeutic agents for COPD with which a compound of formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast, Roflumilast.

Non-limiting examples of therapeutic agents for HCV with which a compound of formula (I) can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b +ribavirin, Ursodeoxycholic Acid, Glycyrrhizic Acid, Thymalfasin, Maxamine, VX-497 and any compounds that are used to treat HCV through intervention with the following targets: HCV polymerase, HCV protease, HCV helicase, HCV IRES (internal ribosome entry site).

Non-limiting examples of therapeutic agents for Idiopathic Pulmonary Fibrosis with which a compound of formula (I) can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil, Interferon-gamma-1β.

Non-limiting examples of therapeutic agents for Myocardial Infarction with which a compound of formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hcl/mag carb, bumetanide, alteplase, enalaprilat, amiodarone hydrochloride, tirofiban hcl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutarnine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, cariporide.

Non-limiting examples of therapeutic agents for Psoriasis with which a compound of formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine.

Non-limiting examples of therapeutic agents for Psoriatic Arthritis with which a compound of formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab.

Non-limiting examples of therapeutic agents for Restenosis with which a compound of formula (I) can be combined include the following: sirolimus, paclitaxel, everolimus, tacrolimus, ABT-578, acetaminophen.

Non-limiting examples of therapeutic agents for Sciatica with which a compound of formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate, temazepam.

Preferred examples of therapeutic agents for SLE (Lupus) in which a compound of formula (I) include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. A compound of formula (I) may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. A compound of formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (PCT Publication No. WO 97/29131; HUMIRA™), CA2 (REMICADE™), CDP 571, TNFR-Ig constructs, (p75TNFRIgG (ENBREL™) and p55TNFRIgG (LENERCEPT™)).

In this invention, the following definitions are applicable:

A “therapeutically effective amount” is an amount of a compound of Formula I or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of formula I which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of formula I and their salts may exist in more than one crystal form and the present invention includes each crystal form and mixtures thereof.

Certain compounds of formula I and their salts may also exist in the form of solvates, for example hydrates, and the present invention includes each solvate and mixtures thereof.

Certain compounds of formula I may contain one or more chiral centers, and exist in different optically active forms. When compounds of formula I contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of formula I contains more than one chiral center it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of formula I may exist in different tautomeric forms or as different geometric isomers, and the present invention includes each tautomer and/or geometric isomer of compounds of formula I and mixtures thereof.

Certain compounds of formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of formula I and mixtures thereof.

Certain compounds of formula I may exist in zwitterionic form and the present invention includes each zwitterionic form of compounds of formula I and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —(CH₂)C(O)H or a moiety that contains a carboxylic acid) wherein the free hydrogen is replaced by (C₁-C₄)alkyl, (C₂-C₁₂)alkanoyloxymethyl, (C₄-C₉) 1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula I wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl”, as used herein, include aromatic and non-aromatic, ring systems, including, but not limited to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azaindole, azetidinyls, benzo(b)thienyl, benzimidazolyl, benzoxazolyl, benzothiazolyl, benzothiadiazolyl, benzoxadiazolyl, furans, imidazoles, imidazolidine, indole, indazoles, isoxazoles, isothiazoles, oxadiazoles, oxazoles, piperazines, piperidines, purine, pyrans, pyrazines, pyrazoles, pyridines, pyrimidines, pyrroles, pyrrolidines, pyrrolo[2,3-d]pyrimidine, pyrazolo[3,4-d]pyrimidine), quinolines, quinazolines, triazoles, thiazoles, tetrahydroindole, tetrazoles, thiadiazoles, thienyls, thiomorpholinos or triazles.

When the term “substituted heterocyclic” (or heterocyclyl) is used, what is meant is that the heterocyclic group is substituted with one or more substituents that can be made by one of ordinary skill in the art and results in a molecule that is a kinase inhibitor. For purposes of exemplification, which should not be construed as limiting the scope of this invention, preferred substituents for the heterocyclyls of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-nitrile, alkynyl, amido groups, amino, aminoalkyl, aminocarbonyl, carbonitrile, carbonylalkoxy, carboxamido, CF₃, CN, —C(O)OH, —C(O)H, —C(O)—)(CH₃)₃, —OH, —C(O)O-alkyl, —C(O)O-cycloalkyl, —C(O)O-heterocyclyl, —C(O)-alkyl, —C(O)-cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, nitro, NO₂, OCF₃, oxo, phenyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)p, cycloalkyl-S(O)p, alkyl-S—, heterocyclyl-S, heterocycloalkyl, cycloalkylalkyl, heterocycolthio, cycloalkylthio, -Z¹⁵⁰—C(O)N(R)₂, -Z¹⁰⁵—N(R)C—(O)-Z²⁰⁰, -Z¹⁰⁵-N(R)—(O)₂-Z²⁰⁰, -Z¹⁰⁵-N(R)—C(O)—N(R)-Z²⁰⁰, —N(R)—C(O)R, —N(R)—C(O) OR, OR—C(O)-heterocyclyl-OR, R_{c} and —CH₂OR_{c};
where R_{c} for each occurrence is independently hydrogen, optionally substituted alkyl, optionally substituted aryl, —(C₁-C₆)—NR_{d}Rₑ, -E-(CH₂)ₜ—NR_{d R}ₑ, -E-(CH₂)ₜ—O-alkyl, -E-(CH₂)ₜ—S-alkyl, or -E-(CH₂),—OH
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;

E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together form a five- or six-membered heterocyclic ring.

An “heterocycloalkyl” group, as used herein, is a heterocyclic group that is linked to a compound by an aliphatic group having from one to about eight carbon atoms. For example, a preferred heterocycloalkyl group is an imidazolylethyl group.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons which are completely saturated. Preferred alkyls are methyl, ethyl, propyl, butyl, pentyl, hexyl and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein, aromatic groups (or aryl groups) include aromatic carbocyclic ring systems (e.g. phenyl and cyclopentyldienyl) and fused polycyclic aromatic ring systems (e.g. naphthyl, biphenylenyl and 1,2,3,4-tetrahydronaphthyl).

As used herein, cycloalkyl means C₃-C₁₂ monocyclic or multicyclic (e.g., bicyclic, tricyclic, etc.) hydrocarbons which is completely saturated or has one or more unsaturated bonds but does not amount to an aromatic group. Preferred examples of a cycloalkyl group are cyclopropyl, cyclobutyl, cyclopentyl, cyclopentenyl, cyclohexyl and cyclohexenyl.

As used herein, amido group means —NHC(═O)—.

As used herein, acyloxy groups are —OC(O)R.

As used herein, many moieties or substituents are termed as being either “substituted” or “optionally substituted”. When a moiety is modified by one of these terms, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinylalkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃ , optionally substituted phenyl, S(O)₂CH₃, S(O)₂CF₃, and sulfonyl, N-alkylamino or N,N-dialkylamino (in which the alkyl groups can also be substituted).

Phamaceutical Formulations

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Routes of Administration.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5 W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinyl-pyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope for the present invention.

The present invention also comprises the use of a compound of formula I as a medicament.

A further aspect of the present invention provides the use of a compound of formula I or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of formula I to a mammal, particularly a human being, in need thereof.

Assays for Screening Compounds of Formula (I)

Enzyme Assays

The in vitro potency of compounds in inhibiting one or more of the protein kinases discussed herein or described in the art may be determined by the procedures detailed below.

The potency of compounds can be determined by the amount of inhibition of the phosphorylation of an exogenous substrate (e.g., a synthetic peptide (Z. Songyang et al., Nature. 373:536-539) by a test compound relative to control.

Enzyme Linked Immunosorbent Assay (ELISA) For PTKs

Enzyme linked immunosorbent assays (ELISA) were used to detect and measure the presence of tyrosine kinase activity. The ELISA were conducted according to known protocols which are described in, for example, Voller, et al., 1980, “Enzyme-Linked Immunosorbent Assay,” In: Manual of Clinical Immunology, 2d ed., edited by Rose and Friedman, pp 359-371 Am. Soc. of Microbiology, Washington, D.C.

The disclosed protocol was adapted for determining activity with respect to a specific PTK. For example, preferred protocols for conducting the ELISA experiments is provided below. Adaptation of these protocols for determining a compound's activity for other members of the receptor PTK family, as well as non-receptor tyrosine kinases, are well within the abilities of those in the art. For purposes of determining inhibitor selectivity, a universal PTK substrate (e.g., random copolymer of poly(Glu₄ Tyr), 20,000-50,000 MW) was employed together with ATP (typically 5 μM) at concentrations approximately twice the apparent Km in the assay.

The following procedure was used to assay the inhibitory effect of compounds of this invention on KDR, Flt-1, Flt-4/VEGFR-3, Tie-1, Tie-2, EGFR, FGFR, PDGFR, IGF-1-R, c-Met, Lck, Blk, Csk, Src, Lyn, Fyn and ZAP70 tyrosine kinase activity:

Buffers and Solutions:

PGTPoly (Glu,Tyr) 4:1

Store powder at −20° C. Dissolve powder in phosphate buffered saline (PBS) for 50 mg/ml solution. Store 1 ml aliquots at −20° C. When making plates dilute to 250 μg/ml in Gibco PBS. Reaction Buffer: 100 mM Hepes, 20 mM MgCl₂, 4 mM MnCl₂, 5 mM DTT, 0.02% BSA, 200 μM NaVO₄, pH 7.10

ATP: Store aliquots of 100 mM at −20° C. Dilute to 20 μM in water

Washing Buffer: PBS with 0.1% Tween 20

Antibody Diluting Buffer: 0.1% bovine serum albumin (BSA) in PBS TMB Substrate: mix TMB substrate and Peroxide solutions 9:1 just before use or use K-Blue Substrate from Neogen

Stop Solution: 1M Phosphoric Acid

Procedure

1. Plate Preparation:

Dilute PGT stock (50 mg/ml, frozen) in PBS to a 250 μg/ml. Add 125 μl per well of Coming modified flat bottom high affinity ELISA plates (Corning #25805-96). Add 125 μl PBS to blank wells. Cover with sealing tape and incubate overnight 37° C. Wash 1× with 250 μl washing buffer and dry for about 2 hrs in 37° C. dry incubator. Store coated plates in sealed bag at 4° C. until used.

2. Tyrosine Kinase Reaction:

Prepare inhibitor solutions at a 4× concentration in 20% DMSO in water.

Prepare reaction buffer

Prepare enzyme solution so that desired units are in 50 μl, e.g. for KDR make to 1 ng/μl for a total of 50 ng per well in the reactions. Store on ice.

Make 4× ATP solution to 20 μM from 100 mM stock in water. Store on ice.

Add 50 μl of the enzyme solution per well (typically 5-50 ng enzyme/well depending on the specific activity of the kinase)

Add 25 μl 4× inhibitor

Add 25 μl 4× ATP for inhibitor assay

Incubate for 10 minutes at room temperature

Stop reaction by adding 50 μl 0.05N HCl per well

Wash plate

Final Concentrations for Reaction: 5 μM ATP, 5% DMSO

3. Antibody Binding

Dilute 1 mg/ml aliquot of PY20-HRP (Pierce) antibody (a phosphotyrosine antibody) to 50 ng/ml in 0.1% BSA in PBS by a 2 step dilution (100×, then 200×)

Add 100 μl Ab per well. Incubate 1 hr at room temp. Incubate 1 hr at 4° C.

Wash 4× plate

4. Color Reaction

Prepare TMB substrate and add 100 μl per well

Monitor OD at 650 nm until 0.6 is reached

Stop with 1 M Phosphoric acid. Shake on plate reader.

Read OD immediately at 450 nm

Optimal incubation times and enzyme reaction conditions vary slightly with enzyme preparations and are determined empirically for each lot.

For Lck, the Reaction Buffer utilized was 100 mM MOPSO, pH 6.5, 4 mM MnCl₂, 20 mM MgCl₂, 5 mM DTT, 0.2% BSA, 200 mM NaVO₄ under the analogous assay conditions.

Homogenous time-resolved fluorescence (HTRF) in vitro kinase assay (see Mathis, G., HTRF(R) Technology. J Biomol Screen, 1999. 4(6): p. 309-314, the contents of which are incorporated in its entirety herein by reference):

Purified enzymes (available from commercial sources) were mixed with different amounts of N-biotinylated substrates or GST-tagged substrates (see table) at varying concentrations of inhibitor in different reaction buffers (40 μL final volume, see table). The kinase reaction was initiated by addition of ATP (0.01-0.1 mM final conc.) in a black 96-half-well plate (Perkin Elmer). After 50-60 minutes incubation at room temperature, the reaction was quenched by addition of EDTA (final conc. 100 μM) and developed by addition of revelation reagents (final approximate concentrations: 30 mM HEPES, pH7.0, 0.06% BSA, 0.006% Tween-20, 0.24 M KF, varying amounts of donor eutropium labeled antibodies and acceptor streptavidin labeled allophycocyanin (SAXL) or anti-GST-XL which are specific to the enzyme reactions. see table). The developed reaction was incubated in the dark either at room temperature for 10 min, or at 4° C. overnight (see table), then read in a time-resolved fluorescence detector (Discovery, Perkin Elmer or Rubystar, BMG) at 620 nm and 665 nm simultaneously. A 337 nm nitrogen laser was used for excitation. The ratio between the signal of 620 nm and 665 nm was used to calculate the IC₅₀.

Specific detailed reaction conditions for the various enzymes are included below:

| | | | | Enzyme | Substrate | ATP | DMSO | Reaction | | |
|---|---|---|---|---|---|---|---|---|---|---|
| | | | Assay | Conc. | Conc. | Conc. | Conc. | Time | Detection | |
| Enzyme | Construct/Mw | Substrate | Buffer | (ng/well) | (μM) | (mM) | (%) | (min) | condition | Comments |
| Akt1 | NA/56 kD | Biotin- | Akt | 0.12 | 4 | 0.1 | 5 | 50 | 13.6 ng/well | Develop at |
| | | Bad- | Buffer | | | | | | Anti-P-BAD- | 4° C. |
| | | peptide | | | | | | | Eu; 0.17 | overnight |
| | | | | | | | | | μg/well | |
| | | | | | | | | | SAXL | |
| B-Raf | Flag-B-Raf | GST- | COT | 30 | 0.15 | 0.1 | 5 | 60 | 14 ng/well | Develop at |
| | (446-766)/37.3 | Unactive | Buffer | | | | | | Anti-P-MEK- | 4° C. |
| | kD | MEK1 | | | | | | | Eu; 0.75 | overnight |
| | | (UBI) | | | | | | | μg/well anti- | |
| | | | | | | | | | GST-XL | |
| Casine Kinase | Human | Biotin- | CKII | 60 | 0.5 | 0.1 | 5 | 60 | 13.6 ng/well | Develop at |
| II | recombinant/ | IκBα- | buffer | | | | | | Anti-P-IκBα- | 4° C. |
| (Calbiochem) | 130 kDa | peptide | | | | | | | Eu; 0.34 | overnight |
| | | | | | | | | | μg/well | |
| | | | | | | | | | SAXL | |
| CDK2/Cyclin | C-His CDK2; | Biotin- | MK2 | 1.335 | 0.1 | 0.1 | 5 | 60 | 15 ng/well | Develop at |
| A (UBI) | N-GST Cyclin | MBP | buffer | | | | | | Anti-P-MBP- | 4° C. |
| | A/110 kD | protein | | | | | | | Eu; 0.34 | overnight |
| | | (UBI) | | | | | | | μg/well | |
| | | | | | | | | | SAXL | |
| CHK1 | CHK1(1-289)- | Biotin- | PKA | 0.6 | 4 | 0.1 | 5 | 60 | 1.8 ng/well | Develop at |
| | His6/33.8 kD | cdc25- | buffer | | | | | | Anti-P-14-3-3 | RT 10 min |
| | | peptide | | | | | | | binding | |
| | | | | | | | | | motif-Eu; | |
| | | | | | | | | | 0.11 μg/well | |
| | | | | | | | | | CR130-100 | |
| COT | Flag-COT30- | Biotin- | COT | 25 | 0.5 | 0.1 | 5 | 60 | 8.6 ng/well | Develop at |
| | 397/45 kD | MEK- | Buffer | | | | | | Anti-P-MEK- | 4° C. |
| | | peptide | | | | | | | Eu; 0.34 | overnight |
| | | | | | | | | | μg/well | |
| | | | | | | | | | SAXL | |
| Erk2 (UBI) | GST-Erk2/ | Biotin- | COT | 1 | 0.05 | 0.1 | 5 | 60 | 15 ng/well | Develop at |
| | 68 kD | MBP | Buffer | | | | | | Anti-P-MBP- | 4° C. |
| | | protein | | | | | | | Eu; 0.34 | overnight |
| | | (UBI) | | | | | | | μg/well | |
| | | | | | | | | | SAXL | |
| IKK1 | His-IKK1/80 | Biotin- | COT | 60 | 0.5 | 0.1 | 5 | 50 | 13.6 ng/well | Develop at |
| | kD | IκBα- | Buffer | | | | | | Anti-P-IκBα- | 4° C. |
| | | peptide | | | | | | | Eu; 0.34 | overnight |
| | | | | | | | | | μg/well | |
| | | | | | | | | | SAXL | |
| IKK2 | His-IKK2/80 | Biotin- | COT | 60 | 0.5 | 0.1 | 5 | 50 | 13.6 ng/well | Develop at |
| | kD | IκBα- | Buffer | | | | | | Anti-P-IκBα- | 4° C. |
| | | peptide | | | | | | | Eu; 0.34 | overnight |
| | | | | | | | | | μg/well | |
| | | | | | | | | | SAXL | |
| JNK1 (UBI) | His-JNK1/45 | Biotin- | MK2 | 40 | 2 | 0.1 | 5 | 60 | 15 ng/well | Develop at |
| | kD | MBP | buffer | | | | | | Anti-P-MBP- | 4° C. |
| | | peptide | | | | | | | Eu; 0.34 | overnight |
| | | (UBI) | | | | | | | μg/well | |
| | | | | | | | | | SAXL | |
| MAPKAPK2 | GST-MK2 | Biotin- | MK2 | 5 | 1 | 0.01 | 5 | 60 | 1.8 ng/well | Develop at |
| | (36-401R)/ | cdc25- | buffer | | | | | | Anti-P-14-3-3 | RT 10 min |
| | 68 kD | peptide | | | | | | | binding | |
| | | | | | | | | | motif-Eu; | |
| | | | | | | | | | 0.11 μg/well | |
| | | | | | | | | | CR130-100 | |
| MAPKAPK3 | GST-MK3 | Biotin- | MK2 | 3 | 1 | 0.1 | 5 | 60 | 1.8 ng/well | Develop at |
| | (35-382)/66.9 | cdc25- | buffer | | | | | | Anti-P-14-3-3 | RT 10 min |
| | KD | peptide | | | | | | | binding | |
| | | | | | | | | | motif-Eu; | |
| | | | | | | | | | 0.11 μg/well | |
| | | | | | | | | | CR130-100 | |
| MEK1 | GST-MEK1- | unactive | COT | 3 | 0.1 | 0.1 | 5 | 60 | 15 ng/well | Develop at |
| (UBI) | His6/71 kDa | Erk2 | Buffer | | | | | | Anti-P-Erk- | 4° C. |
| | | (UBI) | | | | | | | Eu; 0.39 | overnight |
| | | | | | | | | | μg/well Anti- | |
| | | | | | | | | | GST-XL | |
| MEKK3 | Flag-MEKK3 | Unactive | COT | 85 | 40 ng/well | 0.1 | 5 | 50 | 15 ng/well | Develop at |
| | (2-627)/73 kDa | MEK1 | Buffer | | MEK1 | | | | Anti-P-MBP- | 4° C. |
| | | Unactive | | | 250 | | | | Eu; 0.34 | overnight |
| | | Erk2 | | | ng/well | | | | μg/well | |
| | | Biotin- | | | Erk2 | | | | SAXL | |
| | | MBP | | | 0.06 uM | | | | | |
| | | protein | | | Biotin- | | | | | |
| | | (all from | | | MBP | | | | | |
| | | UBI) | | | | | | | | |
| NIK/p100 | Flag-NIK; HA- | Biotin- | COT | 8.5 | 0.5 | 0.1 | 5 | 60 | 13.6 ng/well | Develop at |
| | p100/203.3 kD | IκBα- | Buffer | | | | | | Anti-P-IκBα- | 4° C. |
| | | peptide | | | | | | | Eu; 0.34 | overnight |
| | | | | | | | | | μg/well | |
| | | | | | | | | | SAXL | |
| p38-alpha | GST-p38α/64 | Biotin- | COT | 1.5 | 0.1 | 0.1 | 5 | 60 | 15 ng/well | Develop at |
| (UBI) | kD | MBP | Buffer | | | | | | Anti-P-MBP- | 4° C. |
| | | protein | | | | | | | Eu; 0.34 | overnight |
| | | (UBI) | | | | | | | μg/well | |
| | | | | | | | | | SAXL | |
| PKA | Catalytic subunit, | Biotin- | PKA | 1.6 | 1 | 0.1 | 5 | 60 | 13.6 ng/well | Develop at |
| (Invitrogen) | human | Bad- | buffer | | | | | | Anti-P-BAD- | 4° C. |
| | recombinant/43 | peptide | | | | | | | Eu; 0.17 | overnight |
| | kDa | | | | | | | | μg/well | |
| | | | | | | | | | SAXL | |
| PKC-alpha | His-PKCα/78 | Biotin- | PKC | 0.4 | 2 | 0.1 | 5 | 60 | 1.8 ng/well | Develop at |
| (UBI) | kD | cdc25- | buffer | | | | | | Anti-P-14-3-3 | RT 10 min |
| | | peptide | | | | | | | binding | |
| | | | | | | | | | motif-Eu; | |
| | | | | | | | | | 0.11 μg/well | |
| | | | | | | | | | CR130-100 | |
| PKC-delta | His-PKCδ/77.5 | Biotin- | PKC | 1.5 | 1 | 0.1 | 5 | 60 | 1.8 ng/well | Develop at |
| (UBI) | kD | cdc25- | buffer | | | | | | Anti-P-14-3-3 | RT 10 min |
| | | peptide | | | | | | | binding | |
| | | | | | | | | | motif-Eu; | |
| | | | | | | | | | 0.11 μg/well | |
| | | | | | | | | | CR130-100 | |
| PRAK (UBI) | His-PRAK/54 | Biotin- | MK2 | 40 | 1 | 0.1 | 5 | 60 | 29.2 ng/well | Develop at |
| | kD | MBP | buffer | | | | | | Anti-P-MBP- | 4° C. |
| | | protein | | | | | | | Eu; 0.67 | overnight |
| | | (UBI) | | | | | | | μg/well | |
| | | | | | | | | | SAXL | |
| Raf-1 | Full length | Unactive | COT | 0.1 | 7.5 ng/well | 0.1 | 5 | 60 | 15 ng/well | Develop at |
| (UBI) | human | MEK1 | Buffer | U/well | MEK1 | | | | Anti-P-MBP- | 4° C. |
| | recombinant | Unactive | | | 60 | | | | Eu; 0.34 | overnight |
| | Raf-1/74 kD | Erk2 | | | ng/well | | | | μg/well | |
| | | Biotin- | | | Erk2 | | | | SAXL | |
| | | MBP | | | 0.12 uM | | | | | |
| | | protein | | | Biotin- | | | | | |
| | | (all from | | | MBP | | | | | |
| | | UBI) | | | | | | | | |
| SGK1 (UBI) | His-SGK1 | Biotin- | MK2 | 0.3 | 4 | 0.1 | 5 | 60 | 13.6 ng/well | Develop at |
| | (1-60 a.a. | Bad- | buffer | | | | | | Anti-P-BAD- | 4° C. |
| | deleted, | peptide | | | | | | | Eu; 0.17 | overnight |
| | S422D) | | | | | | | | μg/well | |
| | | | | | | | | | SAXL | |
| cTAK1(UBI) | Full length/ | Biotin- | COT | 30.5 | 4 | 0.1 | 5 | 60 | 1.8 ng/well | Develop at |
| | 90 kDa | cdc25- | Buffer | | | | | | Anti-P-14-3-3 | RT 10 min |
| | | peptide | | | | | | | binding | |
| | | | | | | | | | motif-Eu; | |
| | | | | | | | | | 0.11 μg/well | |
| | | | | | | | | | CR130-100 | |

Reaction Buffers:
COT Buffer:
50 mM Tris-HCl, pH7.5
10 mM MgCl₂
1 mM EGTA
2 mM DTT
0.01% Brij
5 mM Beta-phosphoglycerol
MK2 Buffer:
20 mM MOPS, pH7.2
10 MM MgCl₂
5 mM EGTA
5 mM Beta-phosphoglycerol
1 mM Na₃VO₄
b 0.01% Triton-X-100

01 MM DTT

Akt Buffer:

20 mM HEPES, pH7.5

10 M MgCl₂
0.01% Triton X-100
1 mM DTT
   CKII Buffer:
20 mM Tris, pH7.5
10 mM MgCl₂
10 mM KCl
0.01% Triton-X-100
1 mM DTT
0.5 mM Na₃VO₄
   PKA Buffer:
25 mM HEPES, pH7.4
10 mM MgCl₂
0.01% Triton-X-100

00.5 mM DTT
0.1 mM Na₃VO₄
   PKC Buffer:
20 mM MOPS, pH7.2
10 mM MgCl₂
5 mM EGTA
1.2 mM DTT
0.01% Triton-X-100
10 mM Beta-phosphoglycerol
1.2 mM Nₐ3VO₄
0.1 mg/mL phosphatidylserine
0.01 mg/mL diacylglycerol
0.5 mM CaCl₂
   Substrates:
Biotin-IκBα-peptide: Biotin-Ahx-LDDRHDSGLDSMKDC-amide
Biotin-Bad-peptide: Biotin-EELSPFRGRSRSAPPNLWAAQR-amide
Biotin-CKII-substrate-peptide: Biotin-Ahx-RRADDSDDDDD-amide
Biotin-cdc25-peptide: Biotin-Ahx-AKVSRSGLYRSPSMPENLNRPR
Biotin-MEK-peptide: biotin-AGAGSGQLIDSMANSFVGTR
Biotin-MBP protein, GST-unactive MEK1, unactive Erk2 were all purchased from UBI
   Detection Reagents:
Anti-P-MBP was purchased from UBI, labeled by Cis-Bio International
Anti-P-MEK, Anti-P-BAD, Anti-P-IκBα, Anti-P-Erk were all purchased from Cell-Signaling, and labeled by Cis-Bio International
Anti-P-14-3-3 Binding Motif was purchased from Cell-Signaling, labeled by Perkin Elmer
SAXL was purchased from Prozyme
CR130-100 was purchased from Perkin Elmer
Anti-GST-XL was purchased from Cis-Bio International
   Cellular assays

### Cot Mobility Shift Assay

1) Ms are plated in a 48 well plate at 5.0×10e5 cells/well in a volume of 400 ul. The medium consists of DMEM+0.5% FBS+Gln/Antibiotics. The plates are incubated overnight and the assay is carried out the next day.
2) From a typical dilution plate scheme (i.e. 2 mM cpd. stocks in DMSO, first diluted 1:5 in DMSO, then those individual dilutions diluted 1:4 in DMEM+0.5% FBS for working cpd stocks of 0.16 to 500 μM in 25% DMSO) 16 ul of cpd. is added/well. The final DMSO concentration is 1%. Cpd. range from 0.0064 to 20 uM.
3) Compounds are pre-incubated with cells at 37° C. for 30 min. before stimulation with LPS (E. coli 055:B5) at 100 ng/ml for 30 min.
4) The plate is then placed on ice, the supernatants aspirated off immediately, and the wells are washed with cold PBS.
5) Lysates are immediately prepared with Biorad Cell Lysis [Kit (Cat.# 171-304012)] 75 ul of lysis buffer is added per well and after pipetting up and down 5 times, the plate is shaken at 300 rpm for 20 min. at 4° C. Alternative Lysis buffer is Buffer A (see below for composition).
6) The lysates are transferred to Eppendorf tubes and centrifuged at 16,000 rcf for 10 min. The supernatants are mixed with 2× Sample Buffer and boiled for 5 min. They are kept at −20° C. until use.
7) Gels: 8-16% Novex Tris-gly minigels Cat.# EC60485
   1.5 mm 15 wells 25 ul sample/well
   Run with 2× Tris-Acetate SDS Running Buffer
      Novex Cat.# LA0041
      Final Concentration:
         100 mM Tricine
         100 mM Tris base
         0.2% SDS pH 8.24
   120 Volts for 1.5 hrs.
   100 Volts for 1.5 hrs.
8) Transfer: PVDF membrane Buffer: 10 mM Caps pH 11.0 30 Volts overnight 4° C.
9) Western Blotting conditions:
   Block membranes for 1 hr. in PBS/0.05% Tween20/3% Gelatin
   Blot with primary antibodies in PBS/0.05% Tween20/1% Gelatin for 2 hrs.
   Primary antibodies: COT M-20 at 0.4 ug/ml Santa Cruz (SC-720). pMEK ½ (Ser217/221) at 1:1000, Cell Signaling #9121
   Secondary is Protein A-HRP used 1:2000 for 45 min.
   All washes done with PBS/0.05% Tween20
      Buffer A:
25 mM Tris pH 7.5
150 mM NaCl
1% Trition X-100
20 mM NaF
10 mM Sodium pyrophosphate
1 mM DTT
1 mM EDTA
1 mM EGTA
1 mM Sodium orthovanadate (added fresh)
½ tablet/25 ml Complete EDTA-free. (added fresh) Protease inhibitor cocktail.
Roche 1873580
   HSP27 Cellular Assay in THP-1 Cells

THP-1 cells were serum starved (0.5% FBS) for about 24 hours and seeded to 96 well plates at a density of 5×105 cells /well in 100 ul of low serum media. Test compounds were solubilized in DMSO and added to cells over the range of 25 uM-8 nM (final DMSO conc 0.5%). Compounds were pre-incubated for about 30 mins. before the addition of 1 ug/ml LPS. Cells were stimulated for about 45 mins., washed and lysed in 100 ul of Biorad cell lysis buffer. Level of HSP27 phosphorylation was measured via Bio-Plex phosphoprotein assay utilizing pHSP27 Beadmates from Upstate.

pERK 1 & 2 Cellular Assay in PECs:

Collect PEC's by washing the peritoneal cavity of B6 mice injected 4 days prior with 2 ml of 3% thioglycollate IP.

Wash cells with D-PBS and plate 1×106 cells/0.5 ml/well in 48 well plates in 10% FBS RPMI media supplemented with Penicillin-Streptomycin and 2 mM L-Glutamine.

Grow cells overnight in 370 C CO2 incubator. Change media to 0.5% FBS/media, 0.5 ml/well. Serum starve cells in this media 16 hours in 370C CO2 incubator. Pre-incubate cells and inhibitors (test compounds) (in 1% DMSO/media) 30 minutes. Apply Lipopolysaccharide Escherichia coli (1 mg/ml, Calbiochem, La Jolla, Calif., Catalog Number 437625) to wells and incubate 30 minutes. Wash (with 250 ml/well) and lyse cells (in 100 mL/well) by BioRad Cell Lysis Kit 171-304011. Clear lysates by 2,000 g, 30 minute spin. ERK1/2[pTpY185/187] measurement. Use Biorad Bioplex assay kits, following manufacturer's protocol. Calculate phospho-ERK IC50's for inhibitors tested.

LPS Induced TNF in THP-1 Cells

Thp-1 cells were serum starved (0.5% FBS) for about 24 hours and seeded to 96 well plates at a density of 5×105 cells/well in 100 ul of low serum media. Test compounds were solubilized in DMSO and added to cells over the range of 25 uM-8 nM (final DMSO conc 0.5%). Compounds were pre-incubated for 60 mins before the addition of 1 ug/ml LPS. Cells were stimulated for about 3 hrs. Supernatent media was removed and TNF release was quantified by ELISA. Cellular toxicity was determined by the addition of MTT to the remaining cells.

L PS Induced TNF in Peripheral Blood Mononuclear Cell (PBMC) Assay Protocol:

Prepare PBMC's from leukopak's by Ficoll separation. Adjust the cell density to 1×10⁷ cells/mi in media.

Media used is RPMI Medium 1640 (Gibco BRL, Grand Island, N.Y., Catalog Number 31800) +2% human AB sera (Sigma Chemical Company, St. Louis, Mo., Catalog Number S7148, heat inactivated) with 100 U/ml penicillin (Gibco BRL, Catalog Number 15140), 2mM L-glutamine (Gibco BRL, Catalog Number 25030), 1× MEM Non-Essential Amino Acids (Gibco BRL, Catalog Number 11140), and 10 mM pH 7.3 Hepes. Media is filtered through a 0.2-micron filter unit.

To the wells of 96 well plate(s) apply: 100 uL/well inhibitors (at 2× concentrations) in 1% Dimethyl Sulphoxide, 99% media+100 uL/well PBMC's (1E6 cells/well.)

Pre-incubate cells and inhibitors (test compounds) in 37° C. CO₂ incubator for about 30 minutes.

Apply 10 ng/ml Lipopolysaccharide Escherichia coli (Calbiochem, La Jolla, Calif., Catalog Number 437625) and incubate plate(s) overnight (about 16 hours) in a 37° C. CO₂ incubator to stimulate cytokine production.

Harvest supernates for cytokine analysis: Spin plate(s) in a centrifuge at 180 g for about 10 minutes with no brake to pellet cells (we used a Beckman GPKR centrifuge and spin at 1,000 rpm.) Remove 100 uL/well supernate for cytokine analysis.

For hTNF ELISA, use R&D Systems Catalog Number DTA50 kits and dilute samples about 1/20.

After supernates are harvested, cells are used for MTT Assay to assess compound toxicity.

PBMC MTT Assay to Assess Cellular Toxicity:

MTT is converted into a colored product when it is cleaved by the mitochondrial reductase system, which is present in metabolically active cells. The MTT Assay can be used as a measure of cellular viability.

Follow the LPS induced TNF Peripheral Blood Mononuclear Cell (PBMC) Assay Protocol and harvest supernates for cytokine analysis. Use the remaining PBMC's in 96-well plates for the MTT Assay.

To cells (in about 1×10⁶ cells/100 μL/well) apply 50 μL/well MTT (2.5 mg/ml in D-PBS,3-[4,5-dimethylthiazol-2-yl]-2,5-diphenyl-tetrazolium bromide, Sigma Chemical Company, Catalog Number M-2128) and incubate for 4 hours in a 37° C. CO₂ incubator.

Apply 50 μL/well of 20% Sodium Dodecyl Sulfate (Natriumnlauryl-sulfat, BioRad, Hercules, Calif., Catalog Number 161-0301) and incubate in a 37° C. CO₂ incubator overnight. Read the absorbance at 570 nM-630 nM in an ELISA plate reader. The percent viability of cells is then calculated. Toxicity from putative inhibitor(s) is determined by comparison to a control without inhibitor. This is the 100% viable control (1% DMSO/media+cells+MTT+SDS.)

OD570/630 of sample/OD570/630 of 100% viable control×100=% viability of sample.

LPS Induced TNF in PECs

Collect PEC's (peritoneal exudated cells) by washing the peritoneal cavity of B6 mice injected 4 days prior with 2 ml of 3% thioglycollate IP.

Wash cells with D-PBS and plate 2.5×10⁵/0.25 ml/well in 96 well plates in 10% FBS RPMI media supplemented with Penicillin-Streptomycin and 2 mM L-Glutamine. Grow cells overnight in 37° C. CO₂ incubator. Pre-incubate cells and inhibitors in 1% DMSO/media 0.5% FBS for about 30 minutes. Apply Lipopolysaccharide Escherichia coli (1 μg/ml, Calbiochem, La Jolla, Calif., Catalog Number 437625) and stimulate cells 2 hours in 37° C. C)₂ incubator.

Harvest supernates for cytokine analysis:
Spin plate(s) in a centrifuge at 180 g for about 10 minutes with no brake to pellet cells. Remove 50 uL/well supernate for cytokine analysis.

To measure mTNF cytokine levels, use R & D Systems Catalog Number MTA00 ELISA kits. Calculate TNF IC₅₀.

LPS Induced TNF and IL-10 In Differentiated Human Peripheral Blood Mononuclear Cells (PBMC)

PBMCs are prepared from leukopaks and stored frozen in vials in liquid nitrogen freezer.

Thaw PBMCs and plate in 48 well plates at 2×10⁶ cells per well in 400 μl media (RPMI+2% Hu ab serum+Penicillin/Streptomycin+L-glutamine+non-essential amino acids+Hepes+50 ng/ml Recombinant Human MCSF). Incubate 24 h at 37° C. 5% CO₂. Wash cells 3× with media (no MCSF). In separate 48 well plate, dilute compounds in Media+2% Hu ab serum. For compounds at 10 mM add 10 ul of the compound to 990 μl media then do 1:5 serial dilutions in Media+1% DMSO 200 μl+800 μl media.

Remove media from cells and add 250 μl of compound dilutions in duplicate wells of 48 well plates of cells. To negative and positive control wells, add 250 μl media+1% DMSO. Incubate for about 30 minutes 37° C. 5% CO₂. Stimulate cells with 10 ng/ml LPS for 3 h 30′ at 37° C. 5% CO₂. LPS stock 500g/ml: dilute stock 1:5000 in media then add 25 μl to each well except negative controls which get media alone. Incubate for about 3 hours 30 minutes at 37° C. 5% CO₂.

Add Nigericin (Sigma Cat. # N-7143 FW=747):

(Nigericin Final concentration=20 μM: dissolve 2.7 mg in 805 μl ethanol. Dilute this 1:8 in media 250 μl to 1.75 ml. Add 10 μl/well of 48 well plates.) Incubate 30 minutes 37° C. 5% CO₂. After 30 minutes, remove supernatant to 96 well plates and assay human IL-1β and human TNFα using R & D Systems ELISA Kits.

Compounds of formula I may have therapeutic utility in the treatment of diseases involving both identified, including those not mentioned herein, and as yet unidentified protein tyrosine kinases which are inhibited by compounds of formula I. All compounds exemplified herein significantly inhibit either COT or MK2 at concentrations of 50 micromolar or below.

In Vivo Models

In Vivo Inhibition of LPS-Induced Cytokines

Mice are injected i.v. with LPS (from Escherichia coli Serotype 01111:B4, Sigma #L-4130), dissolved in saline. In order to monitor TNF-α production, 0.1 mpk LPS is given and to measure IFN-γ, IL-1β, IL-18, IL-6, and IL-12, 5 mpk LPS is given. The mice are then cardiac bled for serum at the appropriate time points listed below. The animals are bled at 90 minutes for TNF-α or at 4 hours for IFN-γ, IL-1β, IL-18, IL-6, IL-12, then the serum cytokine levels are measured by ELISA. In compound efficacy studies, the compound is dosed either p.o. or i.p. one hour prior to the LPS injection and the levels of target cytokines are measured and compared with those obtained for the control group in order to calculate ED₅₀ levels.

Compounds can also be tested in animal models of human disease. These are exemplified by experimental auto-immune encephalomyelitis (EAE) and collagen-induced arthritis (CIA). EAE models which mimic aspects of human multiple sclerosis have been described in both rats and mice (reviewed FASEB J. 5:2560-2566, 1991; murine model: Lab. Invest. 4(3):278, 1981; rodent model: J. Immunol 146(4):1163-8, 1991 ). Briefly, mice or rats are immunized with an emulsion of myelin basic protein (MBP), or neurogenic peptide derivatives thereof, and CFA. Acute disease can be induced with the addition of bacterial toxins such as bordetella pertussis. Relapsing/remitting disease is induced by adoptive transfer of T-cells from MBP/peptide immunized animals.

CIA may be induced in DBA/1 mice by immunization with type II collagen (J. Immunol:142(7):2237-2243). Mice will develop signs of arthritis as early as ten days following antigen challenge and may be scored for as long as ninety days after immunization. In both the EAE and CIA models, a compound may be administered either prophylactically or at the time of disease onset. Efficacious drugs should reduce severity and/or incidence.

Certain compounds of this invention which inhibit one or more angiogenic receptor PTK, and/or a protein kinase such as lck involved in mediating inflammatory responses can reduce the severity and incidence of arthritis in these models.

Compounds can also be tested in mouse allograft models, either skin (reviewed in Ann. Rev. Immunol., 10:333-58, 1992; Transplantation: 57(12): 1701-17D6, 1994) or heart (Am. J. Anat.:113:273, 1963). Briefly, full thickness skin grafts are transplanted from C57BL/6 mice to BALB/c mice. The grafts can be examined daily, beginning at day six, for evidence of rejection. In the mouse neonatal heart transplant model, neonatal hearts are ectopically transplanted from C57BL/6 mice into the ear pinnae of adult CBA/J mice. Hearts start to beat four to seven days post transplantation and rejection may be assessed visually using a dissecting microscope to look for cessation of beating.

Certain compounds of this invention which are inhibitors of angiogenic receptor tyrosine kinases can also be shown to be active in a Matrigel implant model of neovascularization. The Matrigel neovascularization model involves the formation of new blood vessels within a clear marble of extracellular matrix implanted subcutaneously which is induced by the presence of proangiogenic factor producing tumor cells (for examples see: Passaniti, A., et al, Lab. Investig. (1992), 67(4), 519-528; Anat. Rec. (1997), 249(1), 63-73; Int. J. Cancer (1995), 63(5), 694-701; Vasc. Biol. (1995), 15(11), 1857-6). The model preferably runs over 3-4 days and endpoints include macroscopic visual/image scoring of neovascularization, microscopic microvessel density determinations, and hemoglobin quantitation (Drabkin method) following removal of the implant versus controls from animals untreated with inhibitors. The model may alternatively employ bFGF or HGF as the stimulus.

The teachings of all references, including journal articles, patents and published patent applications, are incorporated herein by reference in their entirety.

The following examples are for illustrative purposes and are not to be construed as limiting the scope of the present invention.

ABBREVIATIONS

| | |
|---|---|
| Boc | tert-Butoxycarbonyl |
| CDI | N,N′-Carbonyldiimidazole |
| dba | dibenzylidene acetone |
| DBU | 1,8-Diazabicyclo[4.3.0]undec-7-ene |
| DCM | Dichloromethane |
| DIEA | Diisopropylethyl amine |
| DME | 1,2-Dimethoxyethane |
| DMF | N,N-Dimethylformamide |
| DMSO | Dimethyl sulfoxide |
| EtOAc | Ethyl acetate |
| mCPBA | meta-Chloroperbenzoic acid |
| MeOH | Methanol |
| MOMCl | Chloromethyl methyl ether |
| NIS | N-Iodosuccinimide |
| NMP | N-Methyl-2-pyrrolidone |
| r.t. | room temperature |
| TEA | Triethylamine |
| TFA | Trifluoroacetic acid |
| TFAA | Trifluoroacetic anhydride |
| THF | Tetrahydrofuran |
| XANTPHOS | 9,9-Dimethyl-4,5-bis(diphenylphosphino)xanthene |

### GENERAL PROCEDURES AND EXAMPLES

The following examples are ordered according to the ultimate final general procedure used in their preparation. The synthetic routes to any novel intermediates are detailed by sequentially listing the general procedure (letter codes) in parentheses after their name. A worked example of this protocol is given below. Analytical data is defined either within the general procedures or in the tables of examples. Unless otherwise stated, all ¹H or ¹³C NMR data were collected on a Varian Mercury Plus 400 MHz or a Bruker DRX 400 MHz instrument; chemical shifts are quoted in parts per million (ppm). High pressure liquid chromatography (HPLC) analytical data are either detailed within the experimental or referenced to the table of HPLC conditions using the lower case method letter in parentheses provided in Table 1.

TABLE 1
List of HPLC methods

| Method | System | Conditions |
|---|---|---|
| a | 15 min. run | RP-HPLC (5% to 95% acetonitrile/0.05 M aqueous ammonium |
| | (PDA & 486) | acetate, buffered to pH 4.5, over 10 min at 1.7 mL/min; λ = 254 nm; |
| | | Hypersil C18, 100 Å, 5 μm, 250 × 4.6 mm column). |
| b | 15 min. run | RP-HPLC (5% to 100% acetonitrile/0.05 M aqueous ammonium |
| | (PDA & 486) | acetate, buffered to pH 4.5, over 7 min at 1.7 mL/min; λ = 254 nm; |
| | | Hypersil C18, 100 Å, 5 μm, 250 × 4.6 mm column). |
| c | 15 min. run | RP-HPLC (5% to 95% acetonitrile/0.05 M aqueous ammonium |
| | (Alliance) | acetate, buffered to pH 4.5, over 10 min at 1 mL/min; λ = 254 nm; |
| | | Hypersil C18, 100 Å, 5 μm, 250 × 4.6 mm column). |
| d | 20 min. run | RP-HPLC (25% to 100% acetonitrile/0.1 M aqueous |
| | (PDA & 486) | ammonium acetate, buffered to pH 4.5, over 20 min at 1.0 mL/min; |
| | | λ = 254 nm; Hypersil C18, 100 Å, 5 μm, 250 × 4.6 mm |
| | | column). |
| e | 25 min. run | RP-HPLC (50% to 100% acetonitrile/0.05 M aqueous |
| | (PDA) | ammonium acetate, buffered to pH 4.5, over 15 min at 1.7 mL/min; |
| | | λ = 254 nm; Hypersil C18, 100 Å, 5 μm, 250 × 4.6 mm |
| | | column). |
| f | 30 min. run | RP-HPLC (5% to 85% acetonitrile/0.05 M aqueous ammonium |
| | (PDA & 486) | acetate, buffered to pH 4.5, over 20 min at 1.7 mL/min; λ = 254 nm; |
| | | Hypersil C18, 100 Å, 5 μm, 250 × 4.6 mm column). |
| g | 30 min. run | RP-HPLC (5% to 95% acetonitrile/0.05 M aqueous ammonium |
| | (PDA & 486) | acetate, buffered to pH 4.5, over 20 min at 1.7 mL/min; λ = 254 nm; |
| | | Hypersil C18, 100 Å, 5 μm, 250 × 4.6 mm column). |
| h | 30 min. run | RP-HPLC (5% to 85% acetonitrile/0.05 M aqueous ammonium |
| | (Alliance) | acetate, buffered to pH 4.5, over 20 min at 1 mL/min; λ = 254 nm; |
| | | Hypersil C18, 100 Å, 5 μm, 250 × 4.6 mm column). |
| i | AQA & Advantage | RP-HPLC (30% to 95% acetonitrile/0.01 M aqueous ammonium |
| | (normal grad) | acetate, buffered to pH 4.5, over 4.5 min at 0.8 mL/min; λ = 190-700 nm; |
| | | Genesis C18, 120 Å, 4 μm, 33 × 4.6 mm column). |
| j | AQA & Advantage | RP-HPLC (10% to 80% acetonitrile/0.01 M aqueous ammonium |
| | (slow grad) | acetate, buffered to pH 4.5, over 6 min at 0.8 mL/min; λ = 190-700 nm; |
| | | Genesis C18, 120 Å, 3 μm, 30 × 4.6 mm column). |
| k | Prep method | RP-HPLC (20% to 100% acetonitrile/0.05 M aqueous |
| | 25 min | ammonium acetate, buffered to pH 4.5, over 25 min at 15 mL/min; |
| | (254 nM) | λ = 254 nm; Hypersil C18, 100 Å, 8 μm, 250 × 21.2 mm |
| | | column). |
| l | HSA Analytical | RP-HPLC (5% to 95% acetonitrile/0.005 M aqueous ammonium |
| | Agilent (5 min) | acetate, buffered to pH 4.5, over 4.5 min at 2.0 mL/min; diode |
| | | array detector; PECOSPHERE C18, 80 Å, 3 μm, 33 × 4.6 mm |
| | | column). |
| l | HSA Analytical | RP-HPLC (5% to 95% acetonitrile/0.005 M aqueous ammonium |
| | Agilent (5 min) | acetate, buffered to pH 4.5, over 4.5 min at 2.0 mL/min; diode |
| | | array detector; PECOSPHERE C18, 80 Å, 3 μm, 33 × 4.6 mm |
| | | column). |
| m | Analytical | RP-HPLC (0% to 100% acetonitrile/0.05 M aqueous ammonium |
| | (6 min) | acetate, buffered to pH 4.5, over 6 min at 3.5 mL/min; λ = 254 nm; |
| | 254 nM | PECOSPHERE C18, 100 Å, 3 μm, 33 × 4.6 mm column). |
| n | Analytical | RP-HPLC (0%-100% acetonitrile/0.05 M aqueous ammonium |
| | (7 min) | acetate, buffered to pH 4.5, over 7 min at 3.5 ml/min; λ = 254 nm; |
| | 254 nM | PECOSPHERE C18, 100 Å, 3 μm, 33 × 4.6 mm column |
| o | Analytical | RP-HPLC (5% to 100% acetonitrile/0.05 M aqueous ammonium |
| | 15 min run | acetate, buffered to pH 4.5, over 10 min at 1.0 mL/min; λ = 254 nm; |
| | (Varian) | Hypersil C18, 100 Å, 5 μm, 250 × 4.6 mm column). |
| | 254 nM | |
| p | HSA prep method | Column: Waters Xterra Prep MS 19 × 50 mm, 5 u, C18 |
| | | Flow rate: 25 ml/min. Mobile phase: MeCN/5 mM NH4OAc. |
| | | Gradient: Initial 5% B, 1.0 mm. 10%, 6.0 min. 75%, 6.25 min. |
| | | 100%, 7.5 min. 100%, 8.0 min. 5% |
| q | 10 min. run | RP-HPLC (10% to 100% acetonitrile/0.05 M aqueous |
| | (PDA & 486) | ammonium acetate, buffered to pH 4.5, over 5 min at 1.7 mL/min; |
| | | λ = 254 nm; Hypersil C18, 100 Å, 5 μm, 150 × 4.6 mm |
| | | column). |
| r | 15 min. run | RP-HPLC (10% to 100% acetonitrile/0.05 M aqueous |
| | (PDA & 486, | ammonium acetate, buffered to pH 4.5, over 5 min at 1.7 mL/min; |
| | nonpolar) | λ = 254 nm; Hypersil C18, 100 Å, 5 μm, 150 × 4.6 mm |
| | | column). |
| t | 10 min. run | RP-HPLC (5% to 100% acetonitrile/0.05 M aqueous |
| | (PDA & 486) | aminonium acetate, buffered to pH 4.5, over 5 min at 1.7 mL/min; |
| | | λ = 254 nm; Hypersil C18, 100 Å, 5 μm, 250 4.6 mm |
| | | column). |
| u | Prep method | RP-HPLC (20% to 100% acetonitrile/0.05 M aqueous |
| | 25 min | ammonium acetate, buffered to pH 4.5, over 25 min at 15 mL/min; |
| | (254 nM) | λ = 254 nm; Hypersil C18, 100 Å, 8 μm, 250 × 21.2 mm |
| | | column). |

The general synthetic schemes that were utilized to construct the majority of compounds disclosed in this application are described below in (Schemes 1-9).

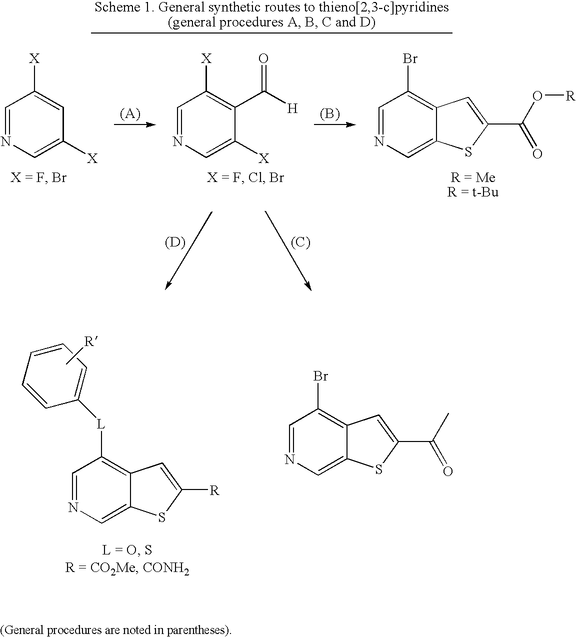

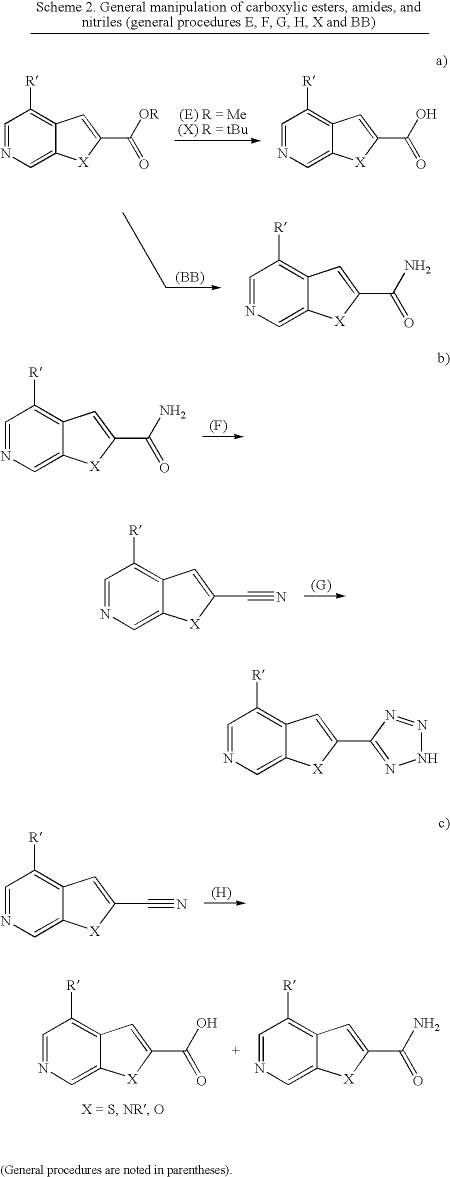

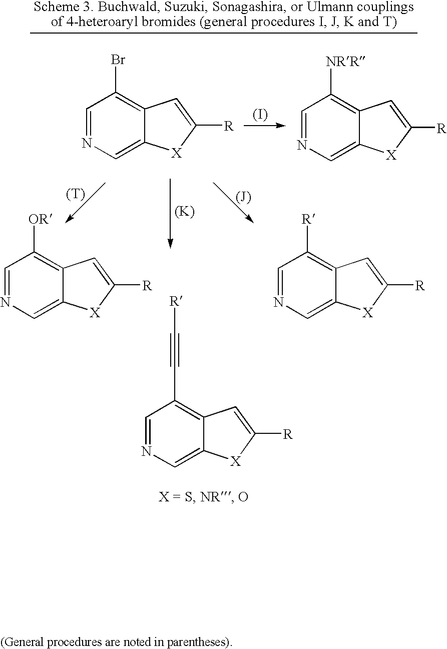

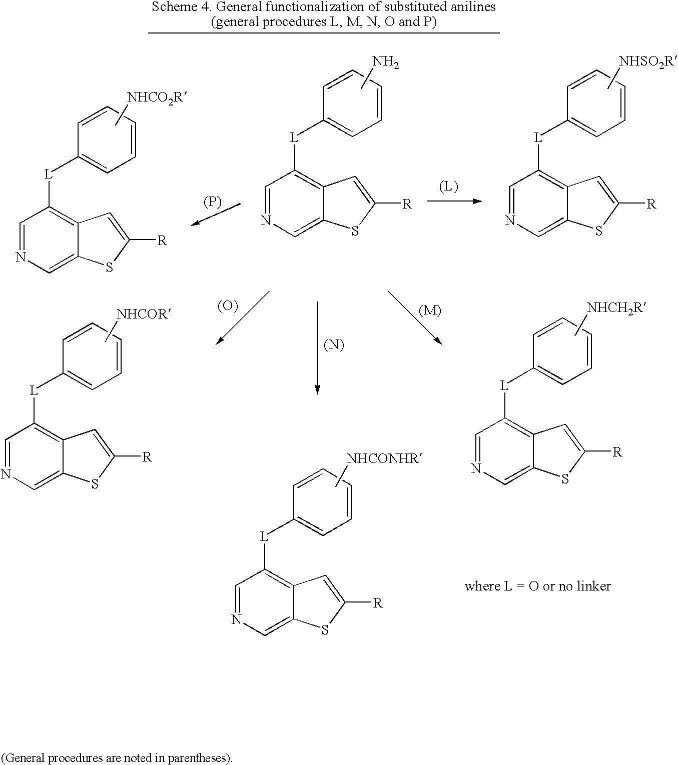

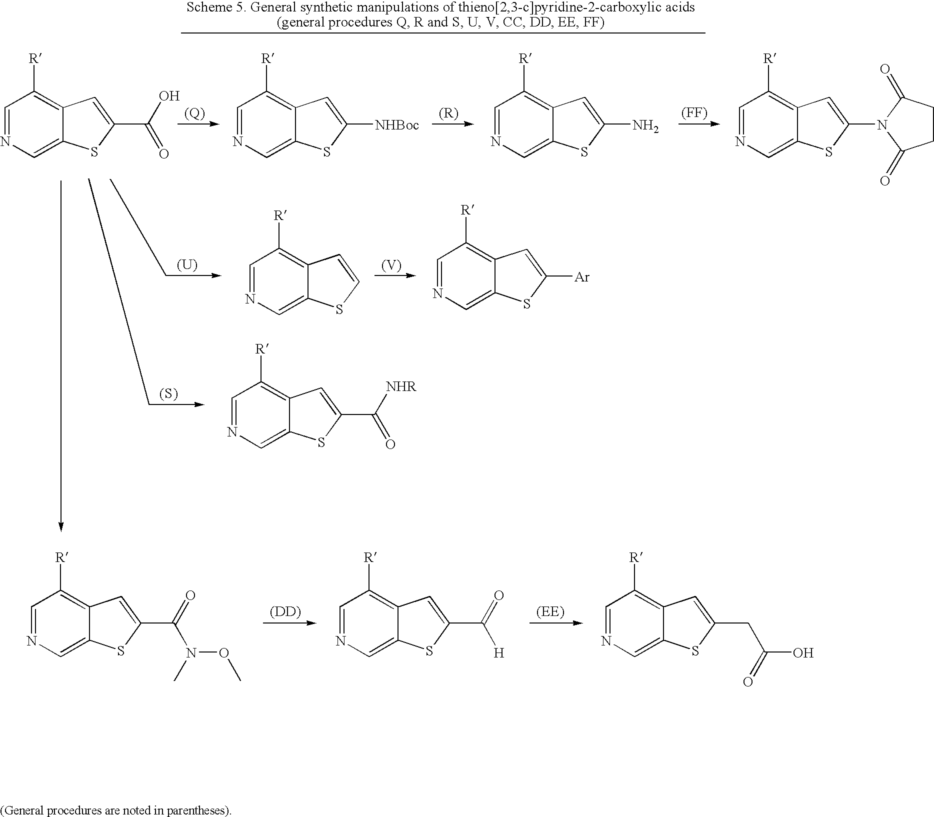

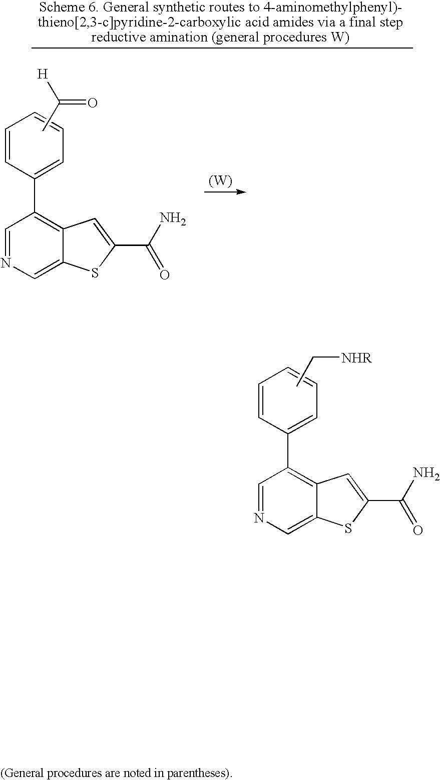

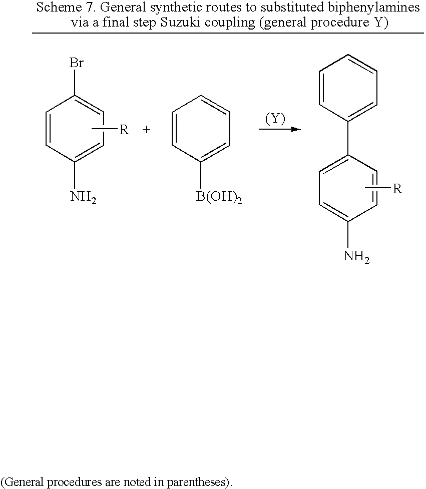

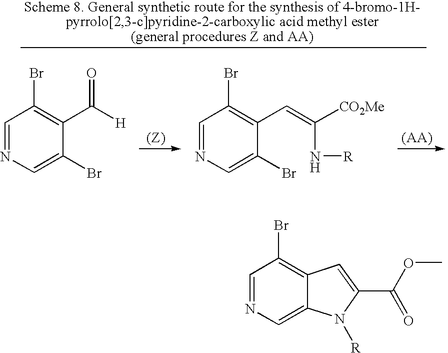

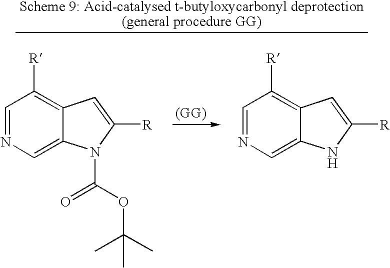

### LIST OF GENERAL PROCEDURES

General Procedure A: Formylation of 3,5-dihalo-pyridines
General Procedure B: Cyclization of a 3-halo-4-formylpyridine with a thioglycolate.
General Procedure C: Cyclization of an ortho halo or nitro arylformate with a thioacetamide
General Procedure D: Synthesis of thieno[2,3-c]pyridine core
General Procedure E: Saponification of a carboxylic ester or nitrile to a carboxylic acid.
General Procedure F: Dehydration of an amide to a nitrile
General Procedure G: Conversion of a nitrile to a tetrazole
General Procedure H: Conversion of carbonitriles to the corresponding amides and carboxylic acids.
General Procedure I: Pd mediated coupling of an aryl halide with an amine or imine.
General Procedure J: Suzuki coupling of a boronate or boronic acid with an aryl halide substrate.
General Procedure K: Sonogashira coupling of an aryl bromide substrate with an alkyne.
General Procedure L: Formation of a sulfonamide from an amine.
General Procedure M: Reductive alkylation of an amine
General Procedure N: Formation of a urea from an amine.
General Procedure O: Acylation of an amine with an acyl chloride or an activated ester
General Procedure P: Formation of a carbamate from an amine.
General Procedure Q: Conversion of a carboxylic acid to the corresponding Boc-amine via a modified Curtius rearrangement.
General Procedure R: Acid catalyzed cleavage of esters and carbamates
General Procedure S: Coupling of an amine to a carboxylic acid to generate an amide, hydroxamate, or hydrazoic acid
General Procedure T: Ullmann coupling reaction for an aryl bromide substrate
General Procedure U: Decarboxylation of a thieno[2,3-c]pyridine-2-carboxylic acid
General Procedure V: Aryl coupling to the 2-position of thieno[2,3-c]pyridines
General Procedure W: Reductive amination aldehydes with amines
General Procedure X: Conversion of a carboxylic acid tert-butyl ester to the carboxylic acid.
General procedure Y: Suzuki coupling of a substituted 4-bromoaniline and a substituted phenylboronic acid via a polymer-bound palladium catalyst
General procedure Z: Horner-Wadsworth-Emmons condensation of benzyloxycarbonylamino-(diethoxy-phosphoryl)-acetic acid methyl ester with aromatic aldehydes.
General procedure AA: Cyclization of 2-protected-amino-3-(3,5-dibromo-pyridin-4-yl)-acrylic acid methyl ester
General Procedure BB: Nucleophilic displacement with an amine
General Procedure CC: Formation of thieno[2,3-c]pyridine-2-carboxylic acid methoxymethyl-amides from the corresponding carboxylic acids
General Procedure DD: Reduction with hydride
General Procedure EE: Preparation of thieno[2,3-c]pyridine-2- acetic acids from the corresponding thieno[2,3-c]pyridine-2-carbaldehyde
General Procedure FF: Condensation of succinic anhydride with 2-amino-thieno[2,3-c]pyridines
General procedure GG: Acid-catalysed t-butyloxycarbonyl deprotection and subsequent saponification.
General procedure HH: Base-promoted nucleophilic substitution
General procedure H: Suzuki coupling of a boronate or boronic acid with an aryl chloride substrate
General procedure JJ: Suzuki coupling of a boronate or boronic acid with an aryl iodide substrate
General procedure KK: Sonagoshira coupling of an aryl halide to an alkyne
General procedure LL: Buchwald coupling of an aryl bromide with an amine
General procedure MM: Sulfonyl urea formation
General Procedure NN: Iodination of thiopyridine
General Procedure OO: Oxidation of a nitrogen or sulfur
General procedure PP: Dehalogenation of an aryl halide
General procedure QQ: Conversion of carboxylate to ester
General procedure RR: Nucleophilic displacement of an aryl halide
General Procedure SS: Dimethyl acetal formation
General procedure TT: Hydrolysis of an acetal
General procedure UU: Addition of a nucleophile to a nitrile
General procedure VV: Heterocycle formation
General procedure WW: Imidazole formation
General procedure XX: Formation of hydroxymethyl imidazole
General procedure YY: Heterocycle formation via the imidate
General procedure ZZ: Addition of a nucleophile to a carbonyl substrate
General procedure AAA: Treatment of N-oxide with phosphorous oxychloride
General Procedure BBB: Preparation of an acid chloride
General procedure CCC: Debromination of an aryl bromide
General procedure DDD: Cyanation of a pyridine N-oxide
General procedure EEE: Mitsunobu coupling
General procedure FFF: Phthalimide deprotection
General procedure GGG: Addition of an isocyanate to an enolate
General procedure HHH: Formation of a 3-aminopyrazole
General procedure III: Reduction of carboxylic acid
General procedure JJJ: Epoxide ring opening with N-hydroxyphthalimide
General procedure KKK: Conversion of Thieno[2,3-c]pyridine N-oxide to 7-Oxo-Thieno[2,3-c]pyridine with optional ester hydrolysis
General procedure LLL: Reductive alkylation of an amine with and aldehyde followed by de-methylation of aromatic methoxy groups
General procedure MMM: Protection of an amine with a Cbz group
General procedure NNN: Wittig olefination reaction
General procedure OOO: Suzuki coupling with in situ generation of borane
General procedure PPP: Stille coupling to aromatic halide
General procedure QQQ: Permanganate oxidation of an aromatic vinyl group
General procedure RRR: Hydrolysis of Imine
General procedure SSS: Amide formation with subsequent deprotection
General procedure TTT: Amide formation with subsequent nucleophilic displacement of an ester
General Procedure UUU: Boronation reaction of an aryl bromide.

The general procedure letter codes constitute a synthetic route to the final product. A worked example of how the route is determined is given below using Example #17 as a non-limiting illustration. The synthesis of Example #17 was completed using general procedure G as detailed in Table 5, i.e.

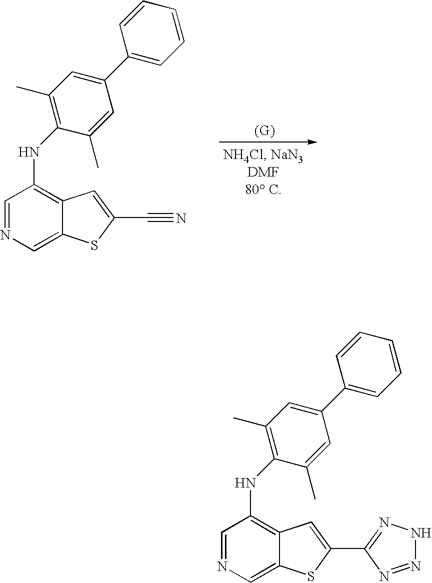

The nitrile was prepared using the route (A, C, F, I(Y)) (as detailed in Table 4). This translates into the following sequence, where the thienopyridine starting material used in general procedure G is the product of following the procedures A, C, F and I, in the given order. In addition, the aniline component used for procedure I is generated following procedure Y, hence this step is designated in additional parentheses.

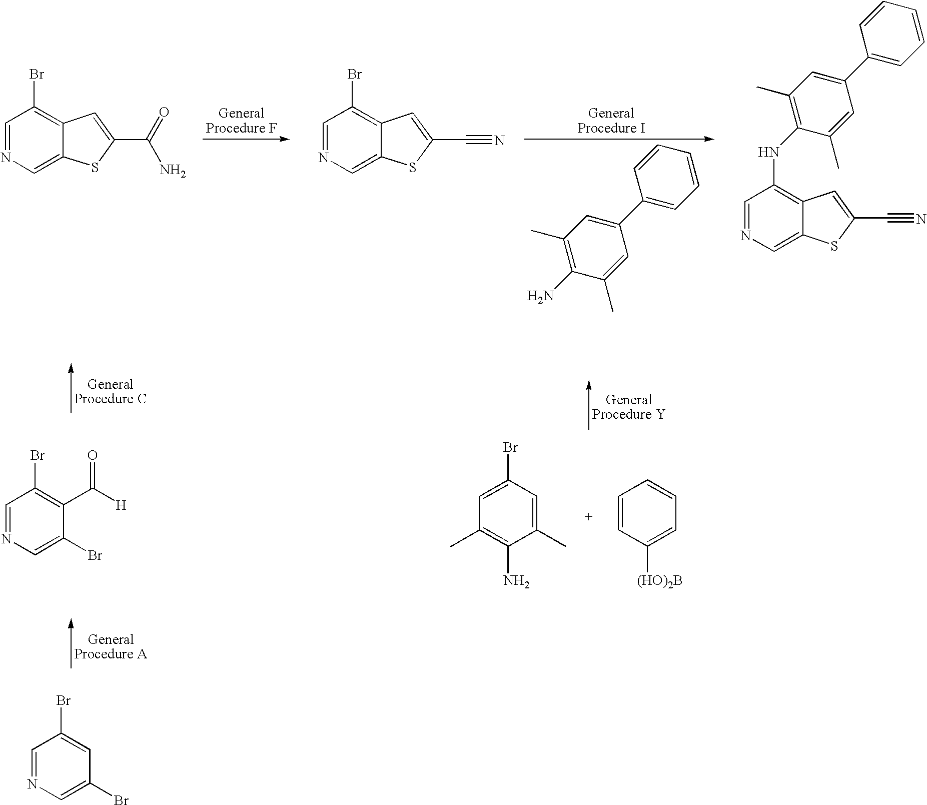

The following describe the synthetic methods illustrated by the foregoing General Procedures schemes and are followed by an example of a compound that was synthesized by the General Procedure. None of the specific conditions and reagents noted in the following are to be construed as limiting the scope of the instant invention and are provided for illustrative purposes only.

### General Procedure A: Formylation of 3,5-dihalo-pyridines

A secondary amine (for example diisopropylamine) (1 to 5 equivalents, preferably 1 equivalent) in an anhydrous solvent (preferably THF) is stirred at about −78 to 30° C. (preferably about 0° C.). A base (for example n-butyllithium) (preferably 1 equivalent) is added at a dropwise rate. The mixture is stirred for about 15 - 60 minutes (preferably 15 min) at about −78 to 30° C. (preferably about 0° C.) then diluted with an anhydrous solvent (preferably THF) and cooled at about −80 to −30° C. (preferably about −78° C.). A solution of 3,5-dihalopyridine (0.7 to 1 equivalent, preferably about 0.9 equivalents) in an anhydrous solvent (preferably THF) is added over 1-4 hours (preferably about 2 hours), while maintaining a reaction temperature at about −80 to −60° C. (preferably about −74° C.). The solution is stirred at about −80 to −30° C. (preferably about −78 ° C.) for about 15-120 minutes (preferably about 30 minutes) and then a formylating agent (for example methyl formate) (1-3 equivalents, preferably about 1.5 equivalents) in an anhydrous solvent (preferably THF) is added such that the reaction temperature is about −80 to −30° C. (preferably about −78° C.). The mixture is stirred for 0.5 to 12 hours (preferably for about 1 hour) at about −80 to −30° C. (preferably about −78° C.) and then transferred into a stirred solution of a weak base such as saturated aqueous NaHCO₃ at about −5 to 25° C. (preferably about 0° C.). The product is extracted with organic solvent (preferably EtOAc) and the combined organic extracts are washed with brine and dried over a dessicant. The solvent is evaporated under reduced pressure to afford the product, which can be further purified by chromatography or crystallization.

Illustration of General Procedure A

### Preparation #1: 3,5-Difluoro-pyridine-4-carboxaldehyde

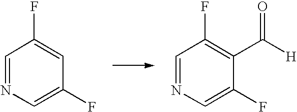

Diisopropylamine (13.4 mL, 95.6 mmol) in THF (40 mL) was stirred at about 0° C. under an atmosphere of nitrogen and n-butyllithium (1.6M in hexanes, 60 mL, 96 mmol) was added while maintaining reaction temperature below about 10° C. The mixture was stirred for about 30 minutes at about 0° C. and then was diluted with THF (150 mL) and cooled to about −78° C. A solution of 3,5-difluoro-pyridine (10.0 g, 86.9 mmol) in THF (100 mL) was added dropwise while maintaining the reaction temperature below about −75° C. The solution was stirred at about −78° C. for about 1 hour and a solution of methyl formate (10.7 mL, 174 mmol) in THF (30 mL) was added over about 30 minutes. The mixture was stirred for about 0.75 hr and then transferred via cannula to a stirred solution of saturated aqueous NaHCO₃ (200 mL) held at about 0° C. The product was extracted with EtOAc (100 mL) and the combined organic extracts were washed with saturated aqueous brine solution (2×100 mL) and dried over anhydrous magnesium sulfate. The solvent was removed under reduced pressure (165 mbar, bath temperature about 30° C.). The crude material was purified by flash chromatography on silica gel using DCM as the mobile phase. Fractions containing the desired product were combined and concentrated under reduced pressure. Crystallization from heptane afforded 3,5-difluoro-pyridine-4-carboxaldehyde as an off-white solid (4.44 g, 31.0 mmol); ¹H NMR (DMSO-d₆, 300 MHz) 10.23 (s, 11H), 8.75 (s, 2H); RP-HPLC (Table 1, Method m) Rₜ 0.62 min.

### General Procedure B: Cyclization of a 3-halo-4-formylpyridine with a thioglycolate

To a solution of a 3-halo-4-formylpyridine (preferably 1 equivalent) in an anhydrous solvent (preferably THF) is added an inorganic base (for example, cesium carbonate or sodium ethoxide, preferably cesium carbonate) (preferably 1.1 equivalents) and a thioglycolate (preferably 1 equivalent). The reaction mixture is heated at about 20-80° C. (preferably about 60° C.) for about 1-16 hours (preferably about 2 hours) then cooled to ambient temperature and concentrated under reduced pressure; or alternatively, partitioned between ice water and an organic solvent and the organic layer is separated. The organic extracts are dried over dessicant. The solvents are evaporated under reduced pressure to afford the product that can be further purified by crystallization or chromatography.

Illustration of General Procedure B

### Preparation #2: 4-Bromo-thieno[2,3-c]pyridine-2-carboxylic acid methyl ester

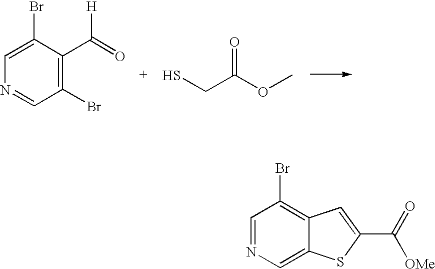

To 3,5-dibromo-pyridine-4-carbaldehyde (prepared using general procedure A) (2.00 g, 7.54 mmol) in THF (75 mL) was added cesium carbonate (2.71 g, 8.29 mmol) and methylthioglycolate (0.800 g, 7.54 mmol). The resulting mixture was heated at about 60° C. for about 2 hours. The reaction mixture was cooled to ambient temperature, poured into ice water, extracted with DCM (2×75 mL), washed with brine (75 mL), dried over magnesium sulfate, filtered, and concentrated to yield 4-bromo-thieno[2,3-c]pyridine-2-carboxylic acid methyl ester as a light yellow solid (1.56 g, 5.73 mmol); ¹H NMR (DMSO-d₆, 400 MHz) δ 9.38 (s, 1H), 8.73 (s, 1H), 8.03 (s, 1H), 4.0 (s, 3H); RP-HPLC (Table 1, Method i), Rₜ 2.90 min; m/z: (M+H)⁺ 272, 274.

### General Procedure C: Cyclization of an ortho halo or nitro arylformate with a thioacetamide

To a solution of a 3-halo-4-formylpyridine (preferably 1 equivalent) in an anhydrous solvent (preferably THF) is added an inorganic base (preferably cesium carbonate) (preferably 1.1 equivalent) and a thioacetamide (preferably 1 equivalent). The resulting mixture is heated at about 60° C. for about 1-6 hours (preferably about 2 hours). The reaction mixture is cooled to ambient temperature and partially concentrated in vacuo. The precipitate is collected by filtration and may be purified by chromatography or crystallization.

Illustration of General Procedure C

### Preparation #3: 4-Bromo-thieno[2,3-c]pyridine-2-carboxylic acid amide

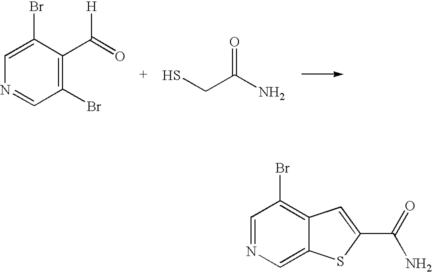

To 3,5-dibromo-pyridine-4-carbaldehyde (prepared using general procedure A) (2.91 g, 11.0 mmol) in THF (110 mL) was added cesium carbonate (3.94 g, 12.1 mmol) and 2-mercaptoacetamide (1.00 g, 11.0 mmol). The resulting mixture was heated at about 60° C. for about 2 hours. The reaction mixture was cooled to ambient temperature and partially concentrated in vacuo. The precipitate was collected by filtration, washed with water, and dried in vacuo to afford 4-bromo-thieno[2,3-c]pyridine-2-carboxylic acid amide (1.17 g, 4.51 mmol) as a off-white solid; (DMSO-d₆, 400 MHz) δ 9.28 (s, 1H), 8.60 (s, 1H), 8.56 (bs, 1H), 8.23 (s, 1H), 7.93 (bs, 1H); RP-HPLC (Table 1, Method i), Rₜ 1.28 min; m/z: (M+H)⁺ 257, 259.

### General Procedure D: Synthesis of thieno[2,3-c]pyridine core

A solution of a phenol or a thiophenol (preferably 2 equivalents) in an anhydrous solvent (preferably THF) is treated with an inorganic base (preferably 2 equivalents) at ambient temperature under inert atmosphere. The mixture is stirred for about 30 minutes-2 hours (preferably about 30 minutes) and then a solution of 3,5-dihalopyridine-4-carboxaldehyde (preferably 1 equivalent) in an anhydrous solvent (preferably THF) is added at room temperature and the mixture is heated at reflux for about 1-4 hours (preferably about 1 hour). The mixture is allowed to cool to ambient temperature and methyl thioglycolate (preferably 1 equivalent) is added and the mixture is refluxed for about 30 minutes. The mixture is cooled to ambient temperature and the solids are removed by filtration. The solvents are removed under reduced pressure to provide crude methyl ester that can be further purified by crystallization or chromatography.

Illustration of General Procedure D

### Preparation #4: 4-Phenylsulfanyl-thieno[2,3-c]pyridine-2-carboxylic acid amide

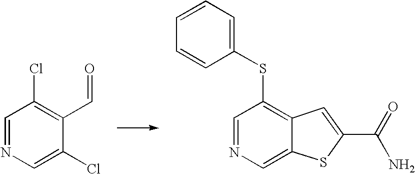

A solution of 3,5-dichloropyridine-4-carboxaldehyde (0.500 g, 2.84 mmol) and thiophenol (0.31 mL, 2.8 mmol) in DMF (10 mL) was treated with potassium carbonate (0.471 g, 3.40 mmol) and the mixture was stirred overnight. The DMF was removed under reduced pressure and the residue was dissolved in DCM, washed with brine, dried over magnesium sulfate, filtered, and concentrated under reduced pressure. The residue was combined with mercaptoacetamide (2.58 mL, 2.84 mmol) and cesium carbonate (1.10 g, 3.40 mmol) in DMF (10 mL) and the mixture was stirred overnight at about 60° C. The mixture was cooled to room temperature and the solvents were removed under reduced pressure. The residue was dissolved in EtOAc and washed with water and saturated aqueous NaCl solution. The residue was purified by preparative RP-HPLC (Hypersil C18, 5 μm, 100 Å, 15 cm; 15%-85% acetonitrile—0.05 M ammonium acetate over 15 min, 1 mL/min) to yield 4-phenylsulfanyl-thieno[2,3-c]pyridine-2-carboxylic acid amide as a yellow solid (0.315 g, 1.10 mmol); RP-HPLC Rₜ 2.12 min (Table 1, Method i); m/z: (M+H)⁺ 287.2.

### General Procedure E: Saponification of a carboxylic ester or nitrile to a carboxylic acid

A mixture of a carboxylic ester (preferably 1 equivalent) in an organic solvent (dioxane, methanol or ethanol, preferably dioxane) and an aqueous inorganic base (lithium hydroxide, sodium hydroxide or potassium hydroxide, preferably NaOH) (preferably 1-4M) is heated at about 20-100° C. (preferably about 70° C.) for about 0.5-60 hours (preferably about 12 hours). The reaction mixture is allowed to cool to ambient temperature and is concentrated in vacuo; or alternatively is acidified to about pH 4 by the addition of aqueous HCl or acetic acid, filtered, washed with water, and dried in vacuo. The product that can be further purified by crystallization or chromatography.

Illustration of General Procedure E

### Preparation #5: 4-(4-Iodo-phenoxy)-thieno[2,3-c]pyridine-2-carboxylic acid

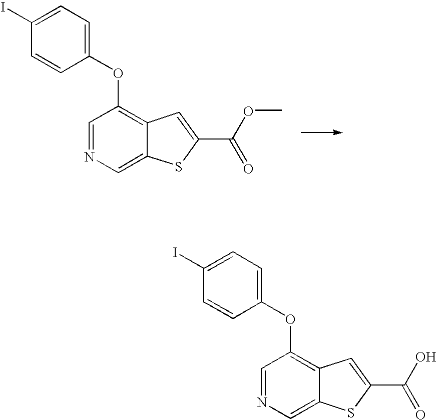

A mixture of 4-(4-iodo-phenoxy)-thieno[2,3-c]pyridine-2-carboxylic acid methyl ester (prepared using general procedure D) (0.050 g, 0.126 mmol) in 1,4-dioxane (1.0 mL) was treated with 2N aqueous NaOH solution (0.20 mL, 0.40 mmol) and heated at about 100° C. in a sealed tube for about 1 hour. The reaction mixture was cooled at ambient temperature, acidified with acetic acid (0.50 mmol), and diluted with water (10 mL). The resulting precipitate was collected by filtration, washed with water, and dried in vacuo to give 4-(4-iodo-phenoxy)-thieno[2,3-c]pyridine-2-carboxylic acid as a white solid (0.042 g, 0.110 mmol); (DMSO-d₆, 400 MHz) δ 9.31 (s, 1H), 8.29 (s, 1H), 7.89 (s, 1H), 7.76 (d, 2H), 6.00 ) d, 2H); RP-HPLC (Table 1, Method i) Rₜ 1.70 min; m/z: (M+H)⁺ 398.

### General Procedure F: Dehydration of an amide to a nitrile

To a 0° C. solution of an amide (preferably 1 equivalent) in organic solvent (pyridine or pyridine/DCM, preferably pyridine) is added TFAA (2-5 equivalents, preferably 2.5 equivalents) rapidly dropwise. The reaction mixture is allowed to warm to ambient temperature over about 2-12 hours (preferably about 6 hours). The pyridine is removed in vacuo and the residue is taken up in DMF; or alternatively partitioned between water and an organic solvent (preferably methylene chloride or ethyl acetate). The organic layer is separated and the aqueous layer is further extracted with organic solvent. The combined organic extracts are dried over a dessicant (preferably sodium or magnesium sulfate). The solvents are removed under reduced pressure to afford the crude product that can be further purified by crystallization or chromatography.

Illustration of General Procedure F

### Preparation #6: 4-Bromo-thieno[2,3-c]pyridine-2-carbonitrile

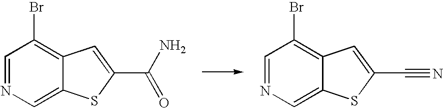

To a 0° C. solution of 4-bromo-thieno[2,3-c]pyridine-2-carboxylic acid amide (prepared using general procedures A, B, and E) (5.0 g, 19 mmol) in pyridine (50 mL) was added TFAA (7.0 mL, 51 mmol) rapidly dropwise. The reaction mixture was allowed to warm to room temperature over 6 h. The pyridine was removed in vacuo and the residue taken up in water (200 mL), extracted with EtOAc (3×500 mL). The combined organic extracts were washed with water (2×100 mL) and brine (100 mL) then dried over sodium sulfate, filtered, and concentrated in vacuo. The crude product was recrystallized from hot EtOAc. Three crops were combined to afford 4-bromo-thieno[2,3-c]pyridine-2-carbonitrile as a white solid (3.50 g, 14.5 mmol): ¹H NMR (DMSO-d₆, 400 MHz) δ 9.44 (s, 1H), 8.77 (m, 1H), 8.50 (d, 1H); RP-HPLC (Table 1, Method i) Rₜ 2.55 min.

### General Procedure G: Conversion of a nitrile to a tetrazole

To a mixture of a nitrile (preferably 1 equivalent) and ammonium chloride (1-2 equivalents, preferably 1.2 equivalents) in DMF is added slowly sodium azide (1-5 equivalents, preferably 1.2 equivalents). The reaction mixture is heated at about 60-85° C. (preferably about 80° C.) for about 2-8 hours (preferably about 3.5 hours) then the temperature is reduced to about 70° C. followed by addition of acetonitrile. The resulting mixture is stirred for about 8-24 hours (preferably about 16 hours) at about 60-75° C. (preferably at about 70° C.) then cooled to ambient temperature. The solvent is partially (about 98%) removed under reduced pressure—care is taken to leave some residual DMF in the flask. To the residue is added water and the resulting solution is acidified to pH 4-5 with the aid of acetic acid. The precipitate is collected by filtration, washed with water, and dried in vacuo to afford the product that can be further purified by crystallization or chromatography.

Illustration of General Procedure G

### Preparation #7: 4-Bromo-2-(2H-tetrazol-5-yl)-thieno[2,3-c]pyridine

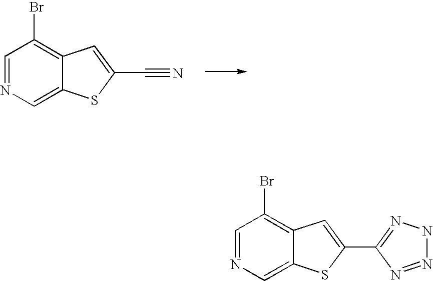

To a stirring solution of 4-bromo-thieno[2,3-c]pyridine-2-carbonitrile (prepared using general procedures A, C, and F) (4.08 g, 17.1 mmol) and ammonium chloride (1.09 g, 20.4 mmol) in DMF (170 mL) was added slowly sodium azide (1.33 g, 20.4 mmol). The reaction mixture was heated at about 80° C. for about 3.5 hours then the temperature was reduced to about 70° C. followed by the addition of acetonitrile (60 mL). The resulting mixture was stirred for about 16 hours at about 70° C. then cooled to ambient temperature. The solvent was partially (about 98%) removed under reduced pressure. Care was taken to leave DMF (about 5-10 mL) in the flask. To the residue was added water (200 mL) and the resulting solution was acidified to pH 4-5 with the aid of concentrated acetic acid. The precipitate was collected by filtration, washed with water, and dried in vacuo to provide 4-bromo-2-(2H-tetrazol-5-yl)-thieno[2,3-c]pyridine as a white solid (4.60 g, 16.1 mmol); ¹H NMR (DMSO-d₆, 400 MHz) δ 9.39 (s, 1H), 8.23 (d, 1H), 8.17 (d, 1H); RP-HPLC (Table 1, Method i), Rₜ 0.63 min.

### General Procedure H: Conversion of carbonitriles to the corresponding amides and carboxylic acids.

To a solution of nitrile (preferably 1 equivalent) in a mixture of dioxane and water (v:v ratio of about 10:1 to 1:10, preferably about 2:1) is added an inorganic base (for example cesium carbonate, sodium carbonate, or potassium hydroxide, potassium t-butoxide, preferably cesium carbonate) (1-3 equivalents, preferably 1 equivalent). The reaction mixture is stirred at about 20-200° C. (preferably about 100° C.) for about 12-48 hours (preferably about 40 hours). The reaction mixture is diluted with about an equal volume of DMF, filtered, and the solvents removed in vacuo. The product can be further purified by chromatography or crystallization.

Illustration of General Procedure H

### Preparation #8: 4-(4-Bromo-phenylamino)-thieno[2,3-c]pyridine-2-carboxylic acid amide and 4-(4-bromo-phenylamino)-thieno[2,3-c]pyridine-2-carboxylic acid

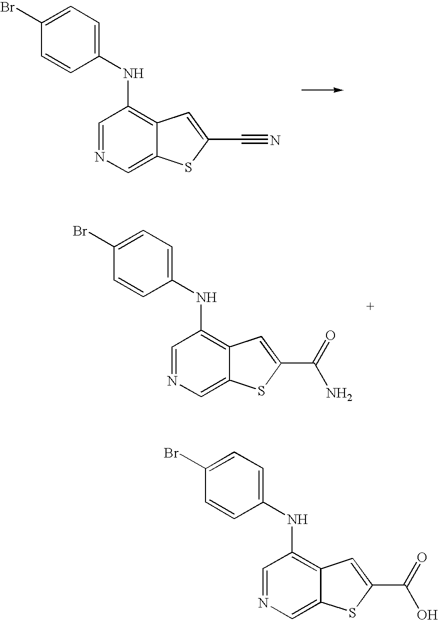

To a solution of 4-(4-bromo-phenylamino)-thieno[2,3-c]pyridine-2-carbonitrile (0.180 g, 0.55 mmol) in dioxane (4 mL) and water (2 mL) was added cesium carbonate (0.178 g, 0.55 mmol). The resulting mixture was heated at about 100° C. for about 24 hours. The reaction mixture was cooled to r.t., diluted with DMF (9 mL), filtered, and purified by preparative RP-HPLC (10% to 60% acetonitrile/0.05M aqueous annmonium acetate, buffered to pH 4.5, over 25 min, then 60% to 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to provide 4-(4-bromo-phenylamino)-thieno[2,3-c]pyridine-2-carboxylic acid amide (0.110 g, 0.35 mmol) as a yellow solid; RP-HPLC Rₜ 8.94 (Table 1, Method a); m/z: (M+H)⁺ 348, 350; and 4-(4-bromo-phenylamino)-thieno[2,3-c]pyridine-2-carboxylic acid (0.021 g, 0.060 mmol) as a yellow solid; RP-HPLC Rₜ 7.30 (Table 1, Method a); m/z: (M+H)⁺ 349, 351.

### General Procedure I: Palladium mediated coupling of an aryl halide with an amine or imine.

A mixture of an aryl halide (preferably 1 equivalent), an aniline or imine (1-3 equivalents, preferably 1 equivalents), an inorganic base (for example cesium carbonate or sodium tert-butoxide, preferably cesium carbonate) (1-20 equivalents, preferably 2 equivalents), and a phosphine ligand (for example XANTPHOS, (±)-2,2′-bis(diphenylphosphino)-1,1′-binaphthalene, (R)-(+)-2,2′-bis(diphenylphosphino)-1,1′-binaphthalene 1,1′-bis(diphenylphosphino)ferrocene, or (S)-(−)-2,2′-bis(diphenylphosphino)-1,1′-binaphthalene, preferably XANTPHOS) (0.05-0.2 equivalents, preferably 0.1 equivalents) is suspended in an anhydrous solvent (for example THF, toluene, 1,4-dioxane, or DMF, preferably 1,4-dioxane) at ambient temperature under an inert atmosphere. Nitrogen gas is bubbled through the suspension for about 5-10 minutes (preferably about 5 minutes). A palladium catalyst (preferably tris(dibenzylideneacetone)dipalladium(0)) (0.02-0.2 equivalents, preferably 0.05 equivalents) is added and nitrogen gas is bubbled through the resulting suspension for about 5-10 minutes (preferably about 5 minutes). The reaction mixture is heated at about 95-110° C. (preferably about 100° C.) for about 1-24 hours (preferably about 12 hours). In the case of an imine coupling, the reaction is cooled to room temperature, opened, dilute aqueous acid (preferably HCl) is added, and the reaction is stirred an additional 12-24 hours (preferably 16 hours). The resulting mixture is allowed to cool to ambient temperature and filtered through a celite pad or alternatively partitioned between an organic solvent (preferably EtOAc) and brine, separated, and dried over a dessicant (preferably magnesium sulfate) and filtered. The solvent is removed in vacuo to give the product that can be further purified by crystallization or chromatography.

Illustration of General Procedure I

### Preparation #9: (5-Phenyl-pyridin-2-yl)-[2-(2H-tetrazol-5-yl)-thieno[2,3-c]pyridin-4-yl]-amine (Example #84)

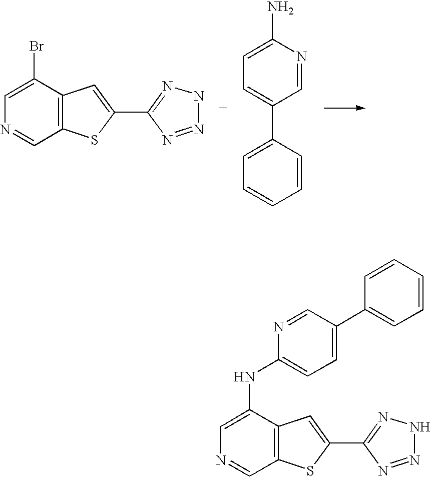

To a solution of 4-bromo-2-(2H-tetrazol-5-yl)-thieno[2,3-c]pyridine (prepared using general procedures A, C, F, and G) (0.100 g, 0.354 mmol) in anhydrous DMF (2 mL) was added 5-phenyl-pyridin-2-ylamine (0.075 g, 0.44 mmol), cesium carbonate (0.232 g, 0.712 mmol), and XANTPHOS (0.021 g, 0.036 mmol). The mixture was stirred and nitrogen gas was bubbled through the suspension for about five minutes at ambient temperature. Tris(dibenzylideneacetone)dipalladium (0) (0.016 g, 0.018 mmol) was added. Nitrogen gas was then bubbled through the resulting mixture for five minutes and the reaction was heated at about 110° C. for about 18 hours. The reaction mixture was cooled to ambient temperature, diluted with DMF (3 mL) and filtered through a Celite® pad. The crude filtrate was purified via preparative RP-HPLC (10% to 60% acetonitrile/0.05M aqueous ammonium acetate, buffered to pH 4.5, over 25 min, then 60% to 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to afford (5-phenyl-pyridin-2-yl)-[2-(2H-tetrazol-5-yl)-thieno[2,3-c]pyridin-4-yl]-amine (0.086 g, 0.23 mmol) as a bright yellow solid; RP-HPLC Rₜ 7.80 min (Table 1, Method a); m/z: (M+H)⁺ 372.2.

### General Procedure J: Suzuki coupling of a boronate or boronic acid with an aryl halide substrate.

To a mixture of a boronate ester or a boronic acid (1-5 equivalents, preferably 2 equivalents), an aryl halide (for example, an aryl bromide, aryl chloride or an aryl iodide, preferably an aryl iodide) (preferably 1 equivalent) and an inorganic base (for example, potassium fluoride, sodium carbonate or cesium carbonate, preferably cesium carbonate) (2-16 equivalents, preferably 2.5 equivalents) in a degassed organic solvent (for example THF, DME, DMF, 1,4-dioxane, 1,4-dioxane and water or toluene, preferably DMF) is added a palladium catalyst (for example tris(benzylideneacetone)dipalladium (0), tetrakis(triphenylphosphine)palladium(0) or bis(acetato)triphenylphosphinepalladium(II) (˜5% Pd) polymer-bound FibreCat™, [1,1′-Bis(diphenylphosphino)ferrocene]dichloropalladium(II), complex with dichloromethane) (0.01-0.10 equivalents, preferably 0.05 equivalents) and, if necessary, tributylphosphinetetraflouroborate. The reaction mixture is heated at about 40-100° C. (preferably about 80° C.) for about 2-24 hours (preferably about 18 hours) under an inert atmosphere. The reaction mixture is allowed to cool to ambient temperature and filtered. The solvents are removed under reduced pressure to afford the product that can be further purified by chromatography or crystallization.

Illustration of General Procedure J

### Preparation #10: 4-Biphenylen-1-yl-2-(1H-tetrazol-5-yl)-thieno[2,3-c]pyridine

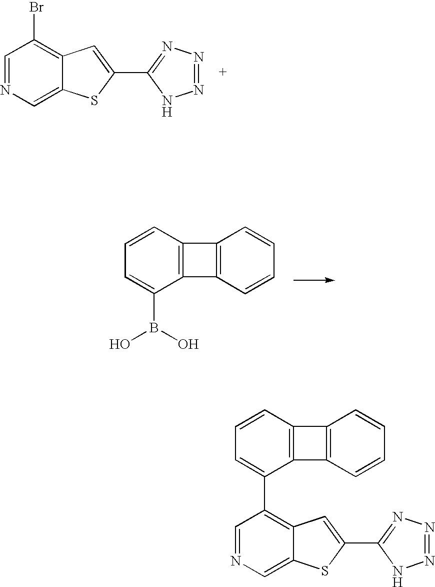

To a mixture of 4-bromo-2-(1H-tetrazol-5-yl)-thieno[2,3-c]pyridine (prepared using general procedures A, C, F, and G) (0.080 g, 0.28 mmol), 1-biphenylenylboronic acid (0.083 g, 0.43 mmol) and cesium carbonate (2N in DMF or water, 1.0 mL, 2.0 mmol) in degassed DMF (5.0 mL) was added tetrakis(triphenylphosphine)palladium(0) (0.016 g, 0.014 mmol) at room temperature under an atmosphere of nitrogen. The reaction mixture was heated at about 80° C. for about 18 hours. The mixture was allowed to cool to ambient temperature, filtered through a Celite® pad, and the solvents were removed under reduced pressure. The residue was purified by preparative RP-HPLC (20%-100% acetonitrile/0.5M aqueous ammonium acetate, buffered to pH 4.5, over 35 min at 15 mL/min; λ=254 nm; Hypersil C18, 100 Å, 8 □m, 250×21.2 mm column) to give 4-biphenylen-1-yl-2-(1H-tetrazol-5-yl)-thieno[2,3-c]pyridine (0.030 g, 0.085 mmol); ¹H NMR (d₆-DMSO, 400 MHz): δ 9.41 (1H, s), 8.60 (1H, s), 8.09 (1H, s), 7.13 (1H, d), 7.06 (1H, dd), 6.92 (1H, d), 6.86 (2H m), 6.73 (1H, m), 6.29 (1H, d); RP-HPLC (Table 1, Method m) Rₜ 2.85 min; m/z: (M+H)⁺ 354.

### General Procedure K: Sonogashira coupling of an aryl bromide substrate with an alkyne.

To a mixture of an aryl bromide (preferably 1 equivalent), alkyne (1-1.5 equivalents, preferably 1 equivalent), organic base (preferably triethylamine) (2-3 equivalents, preferably 2 equivalents), and copper iodide (0.1-0.5 equivalents, preferably 0.2 equivalents) in an anhydrous solvent (for example, THF or DMF, preferably THF) is added a palladium source (preferably tetrakis(triphenylphosphine) palladium(0)) (preferably 5-10 mol %). The resulting mixture is heated at about 70° C. for about 6-12 hours (preferably about 8 hours). The solvent is removed under reduced pressure and the resulting residue is partitioned between an organic solvent and an aqueous solution. The organic layer is separated and the aqueous layer is further extracted with the same organic solvent. The combined organic extracts are dried over a desiccant. The solvent is evaporated under reduced pressure to afford the crude product, which can be further purified by chromatography or crystallization.

Illustration of General Procedure K:

### Preparation #11: 4-Phenethynyl-thieno[2,3-c]pyridine-2-carboxylic acid amide

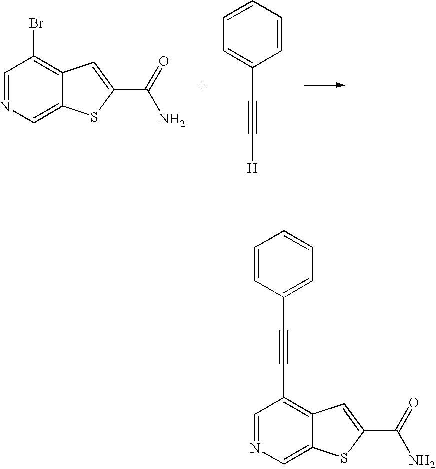

To a mixture of 4-bromo-2-carboxamide[2,3-c]thienopyridine (prepared using general procedures A and C) (0.100 g, 0.367 mmol), phenylacetylene (0.040 mL, 0.36 mmol), triethylamine (0.100 mL, 0.734 mmol), and copper iodide (0.014 g, 0.073 mmol)) in THF (10 mL) was added tetrakis(triphenylphosphine) palladium(0) (0.021 g, 0.018 mmol). The resulting mixture was heated at about 70° C. under inert atmosphere for about 8 hours. After cooling to ambient temperature, the solvent was removed in vacuo and the resulting oil was taken up in ethyl acetate (50 mL) and washed with water (2×50 mL), brine (30 mL), and dried over anhydrous magnesium sulfate, then filtered. The solvents were removed in vacuo and the resulting crude solid was purified by preparative RP-HPLC (Hypersil C18, 5 μm, 100 Å, 15 cm; 15%-85% acetonitrile—0.05 M ammonium acetate over 30 min, 21 mL/min) to yield 4-phenethynyl-thieno[2,3-cJpyridine-2-carboxylic acid amide as a beige powder (0.020 g, 0.071 mmol); RP-HPLC (Table 1, Method i) Rₜ 2.46 min; m/z: (M+H)⁺ 279.2.

### General Procedure L: Formation of a sulfonamide from an amine.

A mixture of an amine (preferably 1 equivalent), aryl sulfonyl chloride (1-5 equivalents, preferably 2 equivalents), and a base (for example pyridine or polymer bound PS-morpholine, preferably polymer bound PS-morpholine) (preferably 4 equivalents) is stirred in an organic solvent (for example DCM, DMF, or pyridine, preferably DCM) at room temperature for about 1-18 hours (preferably about 5 hours). The reaction mixture is filtered, if resin was used, and the solvent is removed under reduced pressure to afford the product that can be further purified by chromatography or by scavenging reactants with functionalized resins (for example PS-Trisamine and PS-Isocyanate) (preferably 3 equivalents with respect to reagent being scavenged).

Illustration of General Procedure L

### Preparation #12: 4-(4-Benzenesulfonylamino-phenoxy)-thieno[2,3-c]pyridine-2-carboxylic acid amide

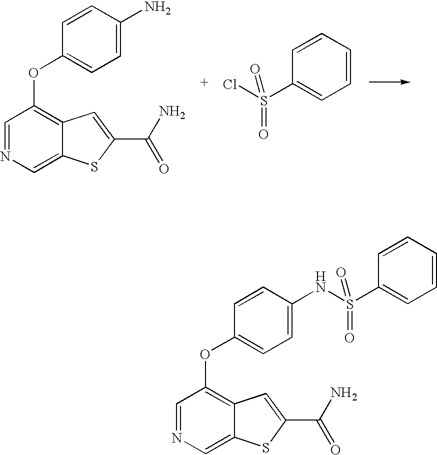

A mixture of 4-(4-amino-phenoxy)-thieno[2,3-c]pyridine-2-carboxylic acid amide (prepared using general procedures A and D) (0.059 g, 0.21 mmol), benzenesulfonyl chloride (0.040 g, 0.23 mmol), and PS-morpholine (0.20 g, 0.83 mmol) was stirred in DMF (2 mL) and DCM (1 mL) at room temperature for about 18 hours. The resin was removed by filtration, and the solvents were removed under reduced pressure to afford the product, which was further purified by preparative RP-HPLC (20%-1 00% acetonitrile/0.05M aqueous ammonium acetate, buffered to pH 4.5, over 45 min at 15 mL/nm; λ=254 nm; Hypersil C18, 100 Å, 8 μm, 250×21.2 mm column) to give 4-(4-benzenesulfonylamino-phenoxy)-thieno[2,3-c]pyridine-2-carboxylic acid amide (0.025 g, 0.059 mmol) as a beige solid; ¹H NMR (d₆-DMSO, 400 MHz) δ 9.06 (1H, s), 8.43 (1H, s), 8.15 (1H, s), 7.94 (2H, d), 7.72 (2H, d), 7.53 (3H, m), 7.06 (4H, dd); RP-HPLC (Table 1, Method m) Rₜ 3.04 min; m/z: (M+H)⁺ 426.4.

### General Procedure M: Reductive alkylation of an amine

To a solution of an amine (preferably 1 equivalent) in an organic solvent (preferably 1,2-dichloroethane) is added an aldehyde (preferably 1 equivalent), sodium triacetoxyborohydride (1-2 equivalents, preferably 1.4 equivalent), and glacial acetic acid (0.5-5 equivalents, preferably 1 equivalent). The mixture is stirred at about 60° C. for about 18 hours under an inert atmosphere. The solvent is removed under reduced pressure to afford the product, which is then triturated in water. The product can be further purified by chromatography or crystallization.

Illustration of General Procedure M

### Preparation #13: 4-[3-(Cyclopropylmethyl-amino)-phenyl]-thieno[2,3-c]pyridine-2-carboxylic acid amide

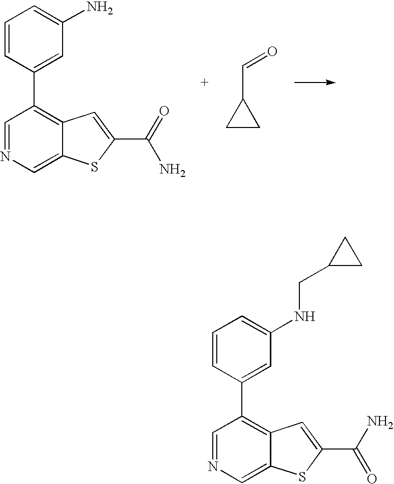

To a solution of 4-(3-amino-phenyl)-thieno[2,3-c]pyridine-2-carboxylic acid amide (prepared using general procedures A, C, and J) (0.150 g, 0.557 mmol) in anhydrous 1,2-dichloroethane (10 mL) was added 2,4-dimethoxy-benzaldehyde (0.093 g, 0.56 mmol), sodium triacetoxyborohydride (0.165 g, 0.779 mmol), and glacial acetic acid (0.033 mL, 0.56 mmol). The mixture was stirred at about 60° C. for about 18 hours under an atmosphere of nitrogen. The solvent was removed in vacuo to afford a residue which was triturated with water and further purified by preparative RP-HPLC (20% to 80% acetonitrile/0.05M aqueous ammonium acetate, buffered to pH 4.5, over 30 min at 21 mL/min; λ=254 nm; Hypersil C18, 100 Å, 8 μm, 250×21.1 mm column) to give 4-[3-(cyclopropylmethyl-amino)-phenyl]-thieno[2,3-c]pyridine-2-carboxylic acid amide (0.388 g, 0.120 mmol) as a yellow solid; RP-HPLC (Table 1, Method a) Rₜ 9.51 min; m/z: (M+H)⁺ 324.

### General Procedure N: Formation of a urea from an amine.

A mixture of an amine (preferably 1 equivalent) and an isocyanate (1-5 equivalents, preferably 2 equivalents) is stirred in an organic solvent (for example DCM, DMF, or pyridine, preferably DCM) at room temperature for about 1-18 hours (preferably about 5 hours). The reaction mixture is filtered, if resin was used, and the solvent is removed under reduced pressure to afford the product that can be further purified by chromatography or by scavenging excess reactants with functionalized resins, (for example, PS-Trisamine or PS-Isocyanate) (preferably 3 equivalents with respect to reagent being scavenged).

Illustration of General Procedure N

### Preparation #14: 4-[4-(3-Phenyl-ureido)-phenoxy]-thieno[2,3-c]pyridine-2-carboxylic acid amide

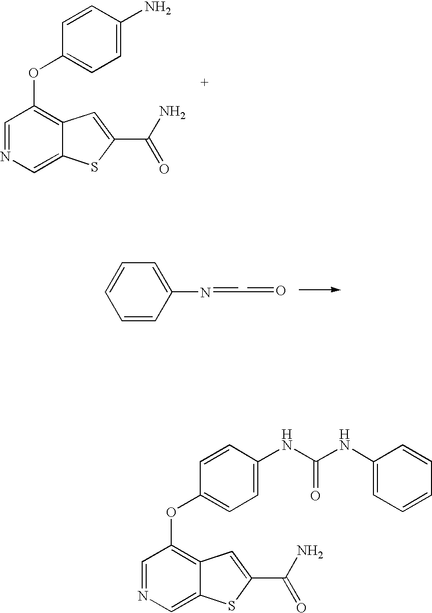

A mixture of 4-(4-amino-phenoxy)-thieno[2,3-c]pyridine-2-carboxylic acid amide (prepared using general procedures A and D) (0.059 g, 0.21 mmol) and phenyl isocyanate (0.027 g, 0.23 mmol) was stirred in anhydrous DMF (2 mL) and anhydrous DCM (1 mL) at room temperature for about 18 hours. The solvents were removed under reduced pressure to afford the product, which was further purified by preparative RP-HPLC (20%-100% acetonitrile/0.05M aqueous anmmonium acetate, buffered to pH 4.5, over 45 min at 15 mL/min; λ=254 nm; Hypersil C18, 100 Å, 8 μm, 250×21.2 mm column) to give 4-[4-(3-phenyl-ureido)-phenoxy]-thieno[2,3-c]pyridine-2-carboxylic acid amide as a beige solid (0.015 g, 0.037 mmol); ¹H NMR (d₆-DMSO, 400 MHz): δ 9.05 (1H, s), 8.48 (1H, s), 8.26 (1H, s), 7.96 (1H, s), 7.86 (1H, s), 7.53 (2H, d), 7.46 (2H, d), 7.27 (2H, t), 7.13 (2H), d), 6.95 (1H, t); RP-HPLC (Table 1, Method m) Rₜ 3.11 min; nm/z: (M+H)⁺ 405.

### General Procedure O: Acylation of an amine with an acyl chloride or an activated ester

Acylation with an acyl chloride: A mixture of an amine (preferably 1 equivalent) and an acyl chloride (preferably 1 equivalent) is stirred in pyridine at ambient temperature for about 1-72 hours (preferably about 18 hours). The solvent is evaporated under reduced pressure to afford the product, which can be further purified by chromatography or crystallization.

Acylation with an activated ester: To a solution of an amine (preferably 1 equivalent) in DMF is added a carboxylic acid (preferably 1 equivalent), 1-(3-dimethoxyaminopropyl)-3-ethylcarbodiimide hydrochloride (1-2 equivalents, preferably 1.7 equivalents), and 1-hydroxy-7-azabenzotriazole (or 1-hydroxybenzotriazole) (preferably 1 equivalent). The mixture is stirred at about 25-40° C. (preferably at about 25° C.) for 18-72 hours (preferably about 18 hours). The solvent is removed under reduced pressure to afford a residue that is triturated with saturated sodium bicarbonate solution and water to afford the product, which can be further purified by chromatography or crystallization.

Illustration of General Procedure O

### Preparation #15: N-{4-[2-(2H-Tetrazol-5-yl)-thieno[2,3-c]pyridin-4-yl]-phenyl}-3-trifluoromethyl-benzamide (Example# 307)

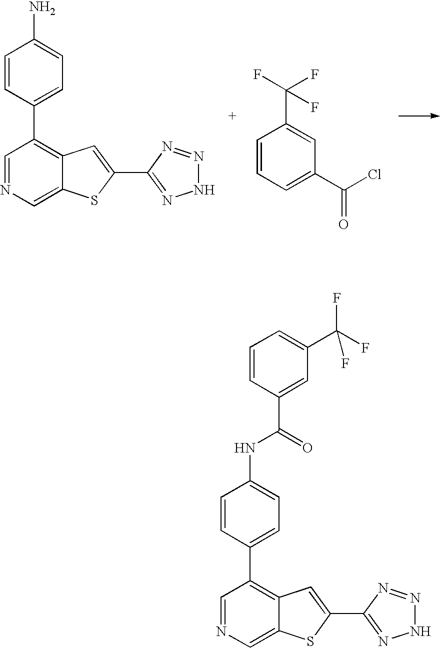

A mixture of 4-[2-(2H-tetrazol-5-yl)-thieno[2,3-c]pyridin-4-yl]-phenylamine (prepared using general procedures A, C, F, G, and J) (0.050 g, 0.17 mmol) and m-(trifluoromethyl)benzoyl chloride (0.026 mL, 0.17 mmol) was stirred at room temperature in pyridine (4 mL) for about 72 hours. The solvent was removed under reduced pressure to afford a residue which was purified by preparative RP-HPLC (10% to 70% acetonitrile/0.05M aqueous ammonium acetate, buffered to pH 4.5, over 30 min at 21 mL/min; λ=254 nm; Hypersil C18, 100 Å, 8 μm, 250×21.1 mm column) to give N-[4-[2-(2H-tetrazol-5-yl)-thieno[2,3-c]pyridin-4-yl]-phenyl}-3-trifluoromethyl-benzamide as a yellow solid (0.023 g, 0.049 mmol); RP-HPLC (Table 1, Method a) Rₜ 8.42 min; m/z: (M+H)⁺ 467.

### General Procedure P: Formation of a carbamate from an amine.

A mixture of an amine (preferably 1 equivalent) and a base (pyridine or inorganic base such as sodium or cesium carbonate, preferably pyridine) is prepared in an organic solvent (THF or pyridine, preferably pyridine). To this mixture is added a chloroformate or alkoxycarbonylanhydride (preferably 1 equivalent) and the reaction is stirred at about ambient temperature to 80° C. for about 6-24 hours (preferably about 12 hours). The solvent is removed under reduced pressure to afford the crude product, which can be triturated in ether; or alternatively partitioned between on organic solvent (preferably EtOAc) and a dilute aqueous inorganic base (preferably sodium bicarbonate) separated from the aqueous layer and dried over a dessicant (sodium or magnesium sulfate, preferably sodium sulfate). The crude product can be further purified by chromatography or crystallization.

Illustration of General Procedure P

### Example #16: [3-(2-Carbamoyl-thieno[2,3-c]pyridin-4-yl)-phenyl]-carbamic acid isopropyl ester

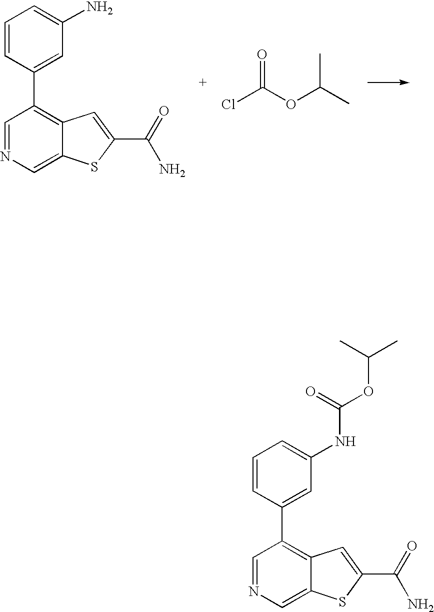

A mixture of 4-(3-amino-phenyl)-thieno[2,3-c]pyridine-2-carboxylic acid amide (prepared using general procedures A, C, and J) (0.099 g, 0.37 mmol) and isopropyl chloroformate (1M in toluene, 0.37 mL, 0.37 mmol) was stirred in pyridine (8 mL) at ambient temperature for about 12 hours. The solvent was removed under reduced pressure to afford the product, which was triturated in ether to afford [3-(2-carbamoyl-thieno[2,3-c]pyridin-4-yl)-phenyl]-carbamic acid isopropyl ester as an off-white solid (0.023 g, 0.065 mmol); RP-HPLC (Table 1, Method a) Rₜ 8.98 min; m/z: (M+H)⁺ 356.

### General Procedure Q: Conversion of a carboxylic acid to the corresponding Boc-amine

A carboxylic acid (preferably 1 equivalent), diphenylphosphoryl azide (preferably 1.1 equivalent), and a tertiary amine (preferably 1.1 equivalents) are combined in an alcoholic solvent (preferably t-butanol) and the mixture is heated at reflux for about 4-30 hours (preferably for about 16 hours) until the reaction is complete by RP-HPLC analysis. The reaction is cooled to ambient temperature, the solvents removed in vacuo, and the product purified by chromatography or crystallization.

Illustration of General Procedure Q

### Preparation #17: [4-(4-Iodo-phenoxy)-thieno[2,3-c]pyridin-2-yl]-carbamic acid tert-butyl ester

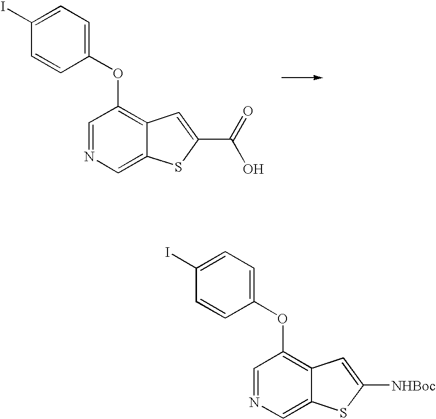

A solution of 4-(biphenyl-4-yloxy)-thieno[2,3-c]pyridine-2-carboxylic acid (prepared using general procedures A, D, and E) (0.347 g, 1.00 mmol), diphenylphosphoryl azide (0.237 mL, 1.10 mmol) and TEA (0.153 mL, 1.10 mmol) were combined in t-BuOH (10 mL) and the mixture was heated at reflux overnight. Additional diphenylphosphoryl azide (0.024 mL, 0.11 mmol) and TEA (0.015 mL, 0.11 mmol) were added and the mixture was heated at reflux for an additional 4 hours. The solvents were removed in vacuo and the resulting residue purified by column chromatography on silica gel using 2:2:1/DCM:heptane:EtOAc as the eluant. Fractions containing the product were combined and concentrated in vacuo to give [4-(4-iodo-phenoxy)-thieno[2,3-c]pyridin-2-yl]-carbamic acid tert-butyl ester as an off-white solid (0.261 g, 0.620 mmol); RP-HPLC (Table 1, Method i) Rₜ=4.03 min; m/z: (M−H)⁻ 417.2.

### General Procedure R: Acid catalyzed cleavage of esters and carbamates

A Boc-protected substrate is dissolved in an organic solvent (preferably TFA, DCM or dioxane) optionally containing a carbonium ion scavenger. If necessary, an inorganic acid is added (HCl or TFA, preferably HCl) and the mixture is stirred at about room temperature until the protecting group has been removed as judged by TLC or HPLC analysis. Solvents are removed under reduced pressure and the product is isolated by crystallization or by chromatography.

Illustration of General Procedure R

### Preparation #18: 4-(4-Iodo-phenoxy)-thieno[2,3-c]pyridin-2-ylamine

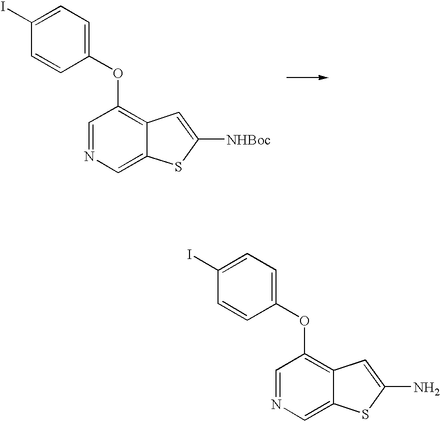

[4-(4-Iodo-phenoxy)-thieno[2,3-c]pyridine-2-yl]-carbamic acid tert-butyl ester (prepared using general procedures A, D, E, and Q) (0.050 g, 0.11 mmol) was stirred in a mixture of TFA (2 mL) and triisopropylsilane (0.023 mL, 0.11 mmol) for about 10 minutes at room temperature. The solvent was removed under reduced pressure and the residue was purified by RP-HPLC (20%-100% acetonitrile/0.05M aqueous ammonium acetate, buffered to pH 4.5, over 25 min at 15 mL/min; π=254 nm; Hypersil C18, 100Å, 8 μm, 250×21.2 mm column) to give 4-(4-iodo-phenoxy)-thieno[2,3-c]pyridin-2-ylamine as an off-white solid (0.022 g, 0.060 mmol); ¹H NMR (d₆-DMSO, 400 MHz): δ 5.61 (s, 1H), 6.70-6.75 (m, 2H), 6.91-6.96 (m, 2H, partially exchanged), 7.62-7.67 (m, 2H), 8.00 (s, 1H), 8.54 (s, 1H); Rₜ=4.03 min; m/z: (M+H)⁺ 468.9.

### General Procedure S: Coupling of an amine to a carboxylic acid to generate an amide, hydroxamate, or hydrazoic acid

A mixture of a carboxylic acid (preferably 1 equivalent), an amine (for example a substituted amine, hydroxylamine, or hydrazine) (1-5 equivalents, preferably 1.1 equivalents), a carbodiimide (for example, 1,3-dicyclohexylcarbodiimide or 1-(3-dimethylaminopropyl)-3-ethylcarbodiimide hydrochloride, preferably 1-(3-dimethylaminopropyl)-3-ethylcarbodiimide hydrochloride] (1-10 equivalents, preferably 1.5 equivalents), a triazole (for example, 1-hydroxybenzotriazole hydrate or 1-hydroxy-7-azabenzotriazole, preferably 1-hydroxy-7-azabenzotriazole) (1-10 equivalents, preferably 1.1 equivalents) and, optionally a base (for example, sodium hydroxide, cesium carbonate, TEA, or diisopropylethylamine, preferably diisopropylethylamine) (1-10 equivalents, preferably 3 equivalents), in an organic solvent (for example, DMF, 1-methyl-2-pyrrolidinone, or 1,4-dioxane, preferably DMF) was stirred between 0-50° C. (preferably at about 20° C.) for about 1-72 hours (preferably about 16 hours). The crude product can further undergo aqueous work-up, chromatography, or crystallization as necessary.

Illustration of General Procedure S

### Preparation #19A: 4-{3-[(Pyridine-4-carbonyl)-amino]-phenyl}-thieno[2,3]pyridine-2-carboxylic acid amide

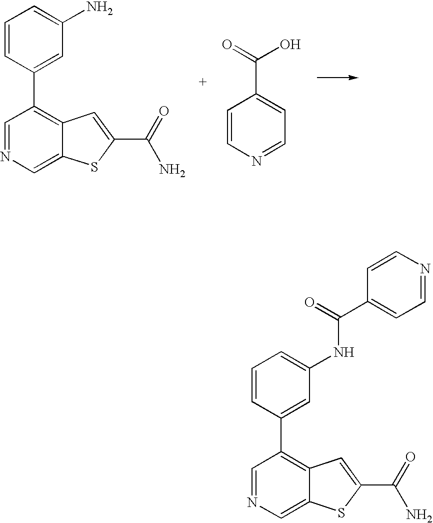

To a solution of 4-(3-amino-phenyl)-thieno[2,3-c]pyridine-2-carboxylic acid amide (prepared using general procedures A, C, and J) (0.100 g, 0.371 mmol) in DMF (10 mL) was added isonicotinic acid (0.048 g, 0.39 mmol), 1-(3-dimethoxyaminopropyl)-3-ethylcarbodiimide hydrochloride (0.121 g, 0.631 mmol), and 1-hydroxy-7-azabenzotriazole (0.053 g, 0.39 mmol). The mixture was stirred at room temperature for 18 hours. The solvent was removed under reduced pressure to afford a residue that was triturated with saturated sodium bicarbonate solution and water. Further purification by preparative RP-HPLC (10% to 60% acetonitrile/0.05M aqueous ammonium acetate, buffered to pH 4.5, over 30 min at 21 mL/min; λ=254 nm; Hypersil C18, 100 Å, 8 μm, 250×21.1 mm column) afforded 4-{3-[(pyridine-4-carbonyl)-amino]-phenyl}-thieno[2,3-c]pyridine-2-carboxylic acid amide (0.040 g, 0.11 mmol); RP-HPLC (Table 1, Method a) Rₜ 7.55 min; m/z: (M+H)⁺ 375.

### Preparation #19: 4-[4-(Biphenyl-4-ylamino)-thieno[2,3-c]pyridine-2-carbonyl]piperazine-1-carboxylic acid tert-butyl ester

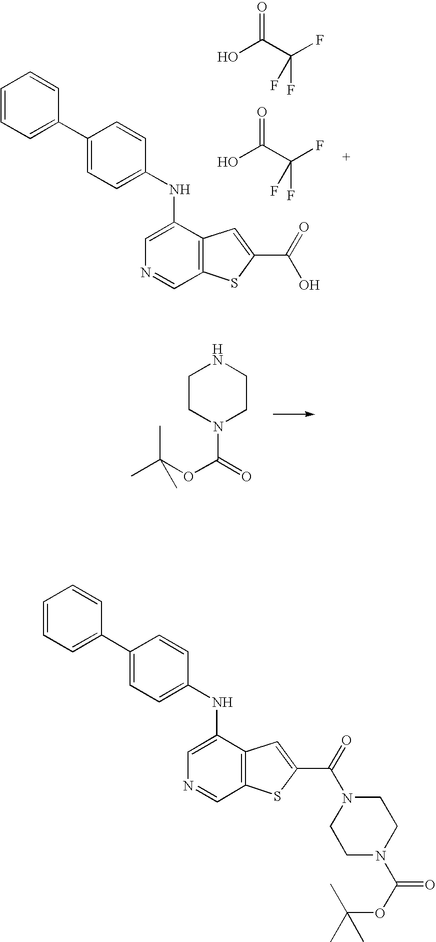

To a solution of 4-(biphenyl-4-ylamino)-thieno[2,3-c]pyridine-2-carboxylic acid ditrifluoroacetate (prepared using general procedures B, I, E) (0.200 g, 0.348 mmol) in DMF (5 mL) was added 1-(3-dimethylaminopropyl)-3-ethylcarbodiimide hydrochloride (0.114 g, 0.595 mmol), 1-hydroxy-7-azabenzotriazole (0.052 g, 0.38 mmol), diisopropylethylamine (0.183 mL, 1.05 mmol) and tert-butyl-1-piperazine carboxylate (0.071 g, 0.38 mmol). The reaction mixture was stirred at room temperature for 16 hours. The crude product was purified by purified by preparative RP-HPLC (5 to 100% acetonitrile in 0.1 M aqueous ammonium acetate over 20 min at 21 mL/min using an 8 μm Hypersil HS C18, 250×21 mm column, λ=254 nm, Rₜ 18.7-20.0 min) to afford 4-[4-(biphenyl-4-ylamino)-thieno[2,3-c]pyridine-2-carbonyl]piperazine-1-carboxylic acid tert-butyl ester as a yellow solid (0.144 g, 0.280 mmol); RP-HPLC (Table 1, Method a) Rₜ 12.08 min; m/z: (M+H)⁺ 515.

### General Procedure T: Ullmann coupling reaction for an aryl bromide substrate

To a mixture of phenol (1-5 equivalents, preferably 2 equivalents), an aryl bromide (preferably 1 equivalent), and an inorganic base (for example, sodium carbonate or cesium carbonate, preferably cesium carbonate) (1-5 equivalents, preferably 2 equivalents) in degassed organic solvent (for example, NMP, dioxane, or toluene, preferably NMP) is added a copper(I) catalyst (for example, cuprous chloride or cuprous iodide, preferably cuprous chloride) (0.1-2.0 equivalents, preferably 0.5 equivalents) and ligand (for example, N-methyl morpholine or 2,2,6,6-tetramethyl-3,5-heptanedione, preferably 2,2,6,6-tetramethyl-3,5-heptanedione) (0.2-4 equivalents, preferably 1.0 equivalent). The reaction mixture is purged and flushed with a dry nitrogen atmosphere about three to five times. The reaction mixture is heated thermally at about 100-150° C. (preferably about 120° C.) for about 3-48 hours (preferably about 18 hours), or heated at about 200-240° C. in a microwave for about 5-20 minutes (preferably about 10 minutes). The mixture is allowed to cool to ambient temperature and the solvent is removed under reduced pressure to afford the product, which can be further purified by chromatography or crystallization.

Illustration of General Procedure T

### Preparation #20A: 4-(2,6-Dimethyl-biphenyl-4-yloxy)-2-(1H-tetrazol-5-yl)-thieno[2,3-c]pyridine

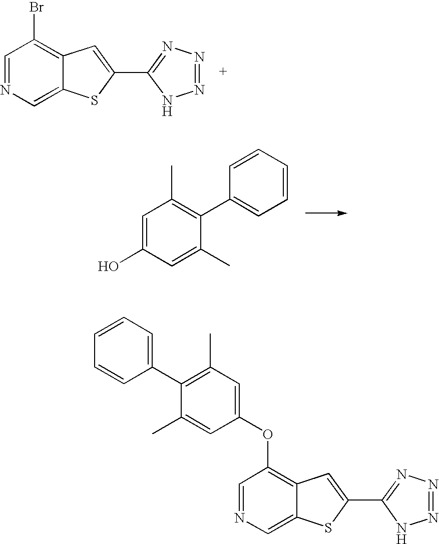

To a mixture of 4-bromo-2-(1H-tetrazol-5-yl)-thieno[2,3-c]pyridine (prepared using general procedures A, C, F, G) (0.129 g, 0.457 mmol), 2,6-dimethyl-biphenyl-4-ol (0.181 g, 0.914 mmol), cesium carbonate (0.354 g, 1.01 mmol), and 2,2,6,6-tetramethyl-3,5-heptanedione (0.050 g, 0.27 mmol) in degassed NMP (10 mL) was added cuprous chloride (0.457 g, 0.457 mmol). The reaction mixture was purged and flushed with nitrogen three times. The reaction mixture was heated at about 120° C. for about 36 hours. The mixture was allowed to cool to ambient temperature, filtered through celite, and the solvent removed under reduced pressure. The residue was purified by preparative RP-HPLC (20% acetonitrile/0.05M aqueous ammonium acetate, buffered to pH 4.5, isocratic for 2 min at 21 mL/min, followed by a gradient of 20%-100% acetonitrile/0.05M aqueous ammonium acetate, buffered to pH 4.5 over 28 min at 21 mL/min; λ=254 nm; Hypersil C18, 100 Å, 8 μm, 250×21.1 mm column) to give 4-(2,6-dimethyl-biphenyl-4-yloxy)-2-(1H-tetrazol-5-yl)-thieno[2,3-c]pyridine (0.015 g, 0.0375 mmol); RP-HPLC (Table 1, Method b), Rₜ 7.41 min; m/z: (M−H)⁻ 398.

### Preparation #20: 4-(3-Isopropoxy-phenoxy)-2-(1H-tetrazol-5-yl)-thieno[2,3-c]pyridine

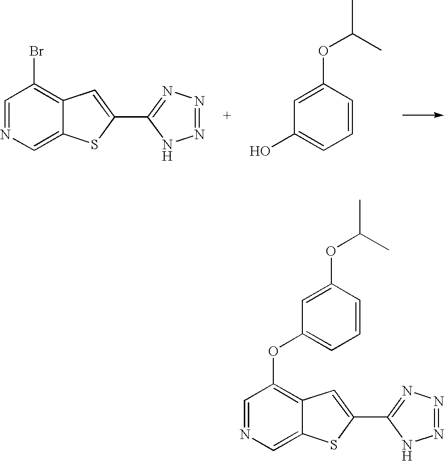

To an Emrys™ Process microwave vial (0.5-2 mL) was added 3-isopropoxy-phenol (0.03 g, 0.3 mmol) dissolved in degassed anhydrous NMP (1 mL) followed by cesium carbonate (0.10 g, 0.30 mmol). Aliquots of the following stock suspensions/solutions were then added respectively: 4-bromo-2-(1H-tetrazol-5-yl)-thieno[2,3-c]pyridine (prepared using general procedures A, C, F, G) (0.50 mL, 0.3 M in NMP, 0.15 mmol), cuprous chloride (0.20 mL, 0.30M in NMP, 0.06 mmol), and 2,2,6,6-tetramethyl-3,5-heptanedione (0.09 mL, 2.0M in NMP, 0.18 mmol). The vial was sealed and the reaction mixture was purged and flushed with nitrogen three times. The reaction was heated with microwave irradiation at about 220° C. for about 10 minutes. The mixture was allowed to cool to ambient temperature and the NMP was removed under reduced pressure. The residue was dissolved in 50% methanol/DMSO (2.5 mL) and purified by preparative RP-HPLC (Table 1, Method p) to give 4-(2,6-dimethyl-biphenyl-4-yloxy)-2-(1H-tetrazol-6-yl)-thieno[2,3-c6pyridine (0.005 g, 0.014 mmol); RP-HPLC (Table 1, Method 1) Rₜ 1.76 min; m/z: (M−H)⁻ 352.4.

### General Procedure U: Decarboxylation of a thieno[2,3-c]pyridine-2-carboxylic acid

A mixture of a thieno[2,3-c]pyridine-2-carboxylic acid (preferably 1 equivalent) and triethylamine (1-5 equivalents, preferably 1 equivalent) is heated in DMF in a resealable tube at about 100-200° C. (preferably about 180° C.) for about 4-24 hours (preferably about 12 hours). The reaction mixture is allowed to cool to ambient temperature and the solvents are removed under reduced pressure to afford the product that can be further purified by crystallization or chromatography.

Illustration of General Procedure U

### Preparation #21: 4-Biphenyl-3-yl-thieno[2,3-c]pyridine

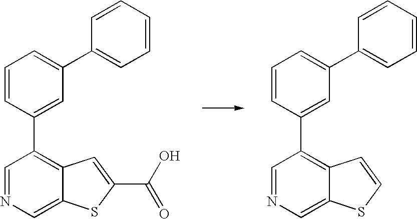

A mixture of 4-biphenyl-3-yl-thieno[2,3-c]pyridine-2-carboxylic acid (prepared using general procedures A, B, J, E) (0.050 g, 0.15 mmol) and triethylamine (0.021 mL, 0.15 mmol) in DMF (1.5 mL) was heated at about 180° C. in a resealable tube for about 16 h. The reaction mixture was cooled to ambient temperature and concentrated in vacuo to give a dark brown oil. Purification by column chromatography on silica gel (elution with 0-5% CH₃OH—CH₂Cl₂) afforded 4-biphenyl-3-yl-thieno[2,3-c]pyridine (0.030 g, 0.10 mmol) as a yellow oil: ¹H NMR (d₆-DMSO, 400 MHz): δ 9.32 (1H, s), 8.59 (1H, s), 8.20 (1H, d), 7.87-7.89 (1H, m), 7.78-7.80 (3H, m), 7.66-7.67 (2H, m), 7.61 (1H, d), 7.48-7.52 (2H, m), and 7.38-7.42 (1H, m); RP-HPLC (Table 1, Method b) Rₜ 10.40 min; m/z: (M+H)⁺ 288.1.

Other products obtained using general procedure U are shown in Table 17. The method used to determine the HPLC retention time is noted in parentheses in a lower case letter that corresponds to a method in Table 1.

### General Procedure V: Aryl coupling to the 2-position of thieno[2,3-c]pyridines

A 2-unsubstituted thieno[2,3-c]pyridine (preferably 1 equivalent) and an aryl halide (preferably 2 equivalents) are combined in a suitable organic solvent (for example, DMF, NMP, 1,4-dioxane, xylenes or DME, preferably DMF) under anhydrous conditions. A palladium catalyst (preferably palladium (II) acetate, preferably 0.05 equivalents), two to four equivalents of an inorganic base (preferably cesium carbonate, preferably three equivalents) and a phosphine ligand (preferably biphenyl-2-yl-di-tert-butyl-phosphane, preferably 0.1 equvialent) are added. Next, molecular sieves can be added (preferably 4 Å). After degassing with nitrogen, the mixture is heated at 50-200° C. (preferably at 150° C.) in a sealed tube for 4-24 h (preferably for 8 hours). The reaction mixture is cooled to ambient temperature, the solids are removed by filtration, and the product is purified by chromatography or crystallization.

Illustration of General Procedure V

### Preparation #22: 4-(Biphenyl-4-yloxy)-2-phenyl-thieno[2,3-c]pyridine

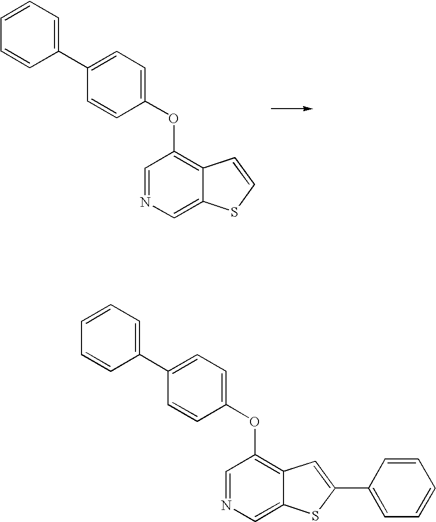

Bromobenzene (0.032 mL, 0.30 mmol), 4-(biphenyl-4-yloxy)-thieno[2,3-c]pyridine (prepared using general procedures A, D, E, and U) (0.050 g, 0.16 mmol), cesium carbonate (0.163 g, 0.500 mmol), biphenyl-2-yl-di-tert-butyl-phosphane (0.012 g, 0.04 mmol) and palladium (II) acetate (0.005 g, 0.02 mmol), and 4 Å molecular sieves (0.250 g) were combined and diluted with DMF (2.0 mL) in a resealable tube. The mixture was purged with nitrogen and heated for about 8 hours at about 150° C. and then cooled to room temperature, filtered, and purified by RP-HPLC (20%-100% acetonitrile/0.05M aqueous ammonium acetate, buffered to pH 4.5, over 25 min at 15mL/min; λ=254 nm; Hypersil C18, 100 Å, 8 μm, 250×21.2 mm column) to give 4-(biphenyl-4-yloxy)-2-phenyl-thieno[2,3-c]pyridine as an off-white solid (0.018 g, 0.048 mmol); RP-HPLC (Table 1, Method b), Rₜ 4.85 min; m/z: (M+H)⁺ 380.2.

### General Procedure W: Reductive amination of an amine with an aldehyde

To a mixture of an aldehyde (preferably 1 equivalent) and amine (preferably 1 equivalent) in an organic solvent (preferably methanol) is added sodium cyanoborohydride (1-5 equivalents, preferably 2.5 equivalents). The resulting solution is heated at about 55° C. for 4-12 hours (preferably about 6 hours). The solvent is removed under reduced pressure and the resulting oil is taken up in organic solvent and washed with water and brine. The combined organic extracts are dried over a dessicant (sodium sulfate or magnesium sulfate, preferably magnesium sulfate) and purified by chromatography or crystallization.

Illustration of General Procedure W

### Preparation #23: 4-{3-[(2-Piperidin-1-yl-ethylamino)-methyl]-phenyl}-thieno[2,3-c]pyridine-2-carboxylic acid amide

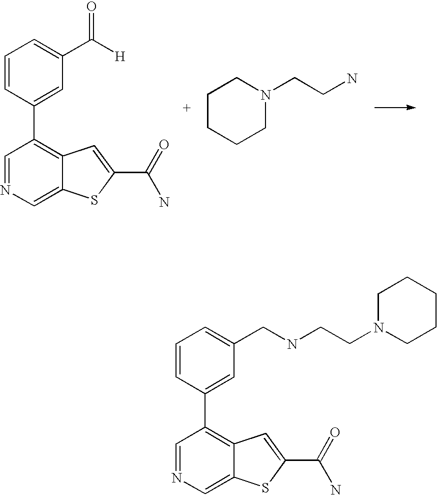

To a mixture of 4-(3-formyl-phenyl)-thieno[2,3-c]pyridine-2-carboxylic acid amide (prepared using general procedures A, C, and J) (0.050 g, 0.17 mmol) and 1-(2-aminoethyl)piperidine (0.025 g, 0.17 mmol) in methanol (3 mL) was added sodium cyanoborohydride (0.027 g, 0.44 mmol). The resulting mixture was heated at about 55° C. for about 12 hours. The reaction mixture was cooled to ambient temperature, the solvent removed in vacuo, and the resulting oil taken up in ethyl acetate (25 mL). The organic portion was separated, washed with water (2×20 mL) and brine (20 mL), dried over magnesium sulfate, filtered, and concentrated in vacuo. The resulting solid was purified by preparative RP-HPLC (Hypersil C18, 5 μm, 100 Å, 15 cm; 15%-85% acetonitrile—0.05 M amnmonium acetate over 30 min, 21 mL/min) to give, 4-{3-[(2-piperidin-1-yl-ethylamino)-methyl]-phenyl]-thieno[2,3-c]pyridine-2-carboxylic acid amide; RP-HPLC (Table 1, Method i), Rₜ 1.00 min; m/z: (M+H)⁺ 395.3.

### General Procedure X: Conversion of a carboxylic acid tert-butyl ester to the carboxylic acid.

A solution of a 4-subsituted-thieno[2,3-c]pyridine-2-carboxylic acid tert-butyl ester (preferably 1 equivalent) in a water-miscible organic solvent (preferably dioxane) and an aqueous solution of ammonia (preferably saturated) (solvent ratios of about 10:1 to 1:1 by volume, preferably 3:1) is sealed in a resealable tube and stirred at temperatures of about 20° C. to 200° C. (preferably about 130° C.). After about 15 hours the reaction mixture is cooled to room temperature, concentrated in vacuo, and purified by chromatography or crystallization.

### Illustration of General Procedure X Preparation #24: 4-(4-Thiophen-3-yl-phenylamino)-thieno[2,3-c]pyridine-2-carboxylic acid

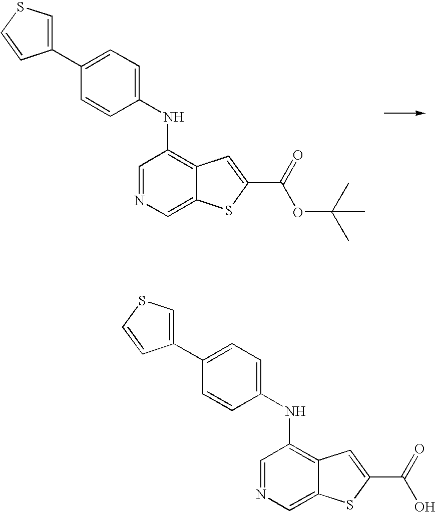

A solution of 4-(4-thiophen-3-yl-phenylamino)-thieno[2,3-c]pyridine-2-carboxylic acid tert-butyl ester (prepared using general procedures A, B, I, and J) (0.141 g, 0.345 mmol) in dioxane (3 mL) and concentrated ammonium hydroxide (1 mL) was sealed in a resealable tube and heated at about 130° C. for about 15 hours. The reaction mixture was cooled to room temperature and concentrated in vacuo. The crude product was purified by preparative RP-HPLC (Rainin Microsorb C18 (model 80-240-C8) 10-40% acetonitrile—0.05 M ammonium acetate over 25 minutes, 21 mL/min) to provide 4-(4-thiophen-3-yl-phenylamino)-thieno[2,3-c]pyridine-2-carboxylic acid (0.020 g, 0.057 mmol) as a yellow solid; ¹H NMR (DMSO-d₆, 400 MHz) δ 8.78 (s, 1H), 8.75 (s, 1H), 8.41-8.40 (m, 2 H), 7.77 (dd, 1H), 7.70-7.68 (d, 2H), 7.63 (dd, 1H), 7.54 (dd, 1H), 7.26 (d, 2H); m/z: (M+H)⁺ 353.

### General procedure Y: Suzuki coupling of a substituted 4-bromoaniline and a substituted phenylboronic acid via a polymer-bound palladium catalyst

A mixture of a substituted 4-bromoaniline (preferably 1 equivalent), a substituted phenylboronic acid (1.0-1.2 equivalents, preferably 1.0 equivalent), an inorganic base (preferably cesium carbonate) (1-3 equivalents, preferably 2 equivalents), and di(acetato)dicyclohexylphenylphosphinepalladium (II) (˜5% Pd) polymer-bound FibreCat TM (1-6 mol %, preferably 4 mol %) is suspended in an organic solvent or mixture of an organic solvent and water (for example ethanol, ethylene glycol dimethyl ether, a mixture of ethanol and water, or a mixture of ethylene glycol dimethyl ether and water, preferably ethanol) in a microwave reaction tube. The resulting suspension is heated at about 100-200° C. (preferably at about 110° C.) for about 10-15 minutes (preferably about 10 minutes). The reaction mixture is cooled to ambient temperature and filtered, washing with an organic solvent (preferably ethanol). The filtrate is concentrated under reduced pressure and the crude material can be carried on to the next step or further purified via crystallization or chromatography.

Illustration of General Procedure Y

### Preparation #25: 3-Methyl-biphenyl-4-ylamine

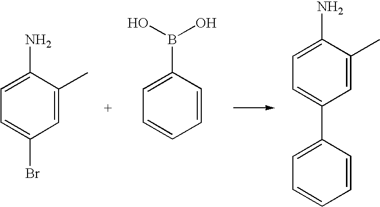

A mixture of 4-bromo-2-methyl-phenylamine (0.153 g, 0.820 mmol), phenylboronic acid (0.100 g, 0.820 mmol), cesium carbonate (0.534 g, 1.64 mmol) and FibreCat® (0.063 g, 4 mol %) was suspended in absolute ethanol (2 mL) in a microwave reaction tube under an ambient atmosphere. The reaction mixture was heated at about 110° C. for about 10 minutes in the microwave. The reaction mixture was cooled to ambient temperature, filtered, and the solid residue was washed with ethanol (about 3 mL). The filtrate was concentrated under reduced pressure to afford 3-methyl-biphenyl-4-ylamine (0.290 g, 1.58 mmol) as a dark brown oil; RP-HPLC Rₜ 10.9 min (Table 1, Method a); m/z (M+H)⁺ 184.1.

### General procedure Z: Horner-Wadsworth-Emmons condensation of benzyloxycarbonylamino-(diethoxy-phosphoryl)-acetic acid methyl ester with aromatic aldehydes.

To an N-protected-amino-(diethoxy-phosphoryl)-acetic acid methyl ester (preferably 1.2 equivalents) in an anhydrous organic solvent (toluene or DCM, preferably DCM) is added a base (preferably DBU, 1-5 equivalents, preferably 1.1 equivalents). The mixture is stirred at room temperature for about 5-30 minutes (preferably for about 15 minutes). If necessary, to this mixture is added dropwise a solution of an aromatic aldehyde (preferably about 1 equivalent) in an anhydrous organic solvent (preferably DCM). The mixture is stirred at about 0-100° C. (preferably at about 20° C.) for about 0.5-24 hours (preferably for about 3 hours) under an inert atmosphere. The solvent is removed in vacuo and the residue is either partitioned between an organic solvent (preferably EtOAc) and an inorganic aqueous acid (preferably about IN HCl) then separated and dried over dessicant (preferably soudium sulfate) and concentrated; or alternatively taken up in an anhydrous mixture of solvent (for example, diethyl ether/heptane, diethyl ether/petroleum ether, diethyl ether/toluene, or EtOAc/heptane, preferably diethyl ether/heptane) followed by isolation of the precipitate by filtration and washing with an anhydrous solvent (for example, diethyl ether, heptane, petroleum ether, or toluene, preferably a mixture of 2:1 heptane/diethyl ether); or alternatively the product is purified directly. The crude product can be further purified by chromatography or crystallization.

Illustration of General Procedure Z

### Preparation #26 Preparation of 2-benzyloxycarbonylamino-3-(3,5-dibromo-pyridin-4-yl)-acrylic acid methyl ester

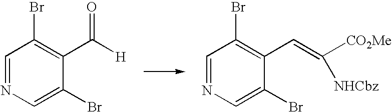

To a solution of N-benzyloxycarbonyl-α-phosphono-glycine trimethyl ester (29.7 g, 89.7 mmol) in anhydrous DCM (500 mL) was added diazabicycloundec-7-ene (0.1 M solution in DCM, 14.6 mL, 97.9 mmol) dropwise. The reaction mixture was stirred for about 20 minutes at room temperature then a solution of 3,5-dibromo-pyridine-4-carbaldehyde (21.5 g, 81.6 mmol) in DCM (300 mL) was added dropwise and the resulting reaction mixture was stirred at room temperature for about 2 hours. The solvent was removed in vacuo and the resulting semi-solid taken up in EtOAc (500 mL) and washed with IN aqueous HCl (3×150 mL). The organic phase was separated, dried over sodium sulfate, and the solvent removed in vacuo. The resulting semi-solid was triturated using a 2:1 mixture of heptane-ethyl ether to provide 2-benzyloxycarbonylamino-3-(3,5-dibromo-pyridin-4-yl)-acrylic acid methyl ester as an off-white powder (32.4 g, 69.1 mmol); ¹H NMR (d₆-DMSO, 400 MHz): δ 9.44 (1H, bs), 8.72 (2H, s), 7.30-7.41 (5H, m), 6.59 (1H, s), 5.05 (2H, s), and 3.74 (3H, s); RP-HPLC (Table 1, Method n) Rₜ 4.18 min (major isomer); m/z: (M+H)⁺ 471.

### General procedure AA: Cyclization of 2-protected-amino-3-(3,5-dibromo-pyridin-4-yl)-acrylic acid methyl ester

A solution of a protected-amino-3-bromo-pyridin-4-yl)-acrylic acid methyl ester (preferably 1 equivalent), a carbonate base (for example, sodium carbonate, potassium carbonate, cesium carbonate, preferably potassium carbonate) (preferably 3 equivalents), and copper (I) (for example, copper (I) iodide, copper (I) bromide, or copper oxide, preferably copper (1) iodide) (preferably 0.05 equivalent) in an anhydrous solvent (for example, dioxane, DMSO, or toluene, preferably toluene) (preferably about 0.08 M) is degassed three times with nitrogen by evacuating and purging. A ligand (for example, N,N-dimethylethylene diamine, N,N′-dimethylethylene diamine, or L-proline, preferably L-proline) is added and the reaction mixture is degassed again and heated at about 20-120° C. (preferably at about 100° C.) for a period of 2-24 hours (preferably for about 8 hours). The reaction is cooled to ambient temperature and the solvent is removed in vacuo. Water is added and the resulting precipitate is collected by filtration. The product may be further purified by chromatography or crystallization.

Illustration of General Procedure AA

### Preparation #27 Preparation of 4-bromo-1H-pyrrolo[2,3-c]pyridine-2-carboxylic acid methyl ester

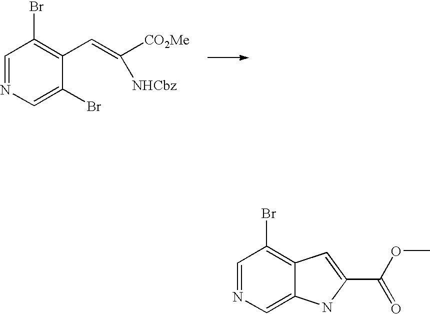

To a mixture of 2-benzyloxycarbonylamino-3-(3,5-dibromo-pyridin-4-yl)-acrylic acid methyl ester (prepared using general procedures A and Z) (2.18 g, 4.63 mmol), potassium carbonate (1.92 g, 13.9 mmol), copper (I) iodide (0.166 g, 0.2 mmol), and L-proline (0.21 g, 1.8 mmol) was added 1,4-dioxane (52 mL) under an inert atmosphere and the resulting heterogenous mixture was heated at about 100° C. for about 20 hours. The reaction was cooled to ambient temperature and the solvent removed in vacuo. Water (50 mL) was added and the resulting precipitate was collected by filtration to afford crude 4-bromo-1H-pyrrolo[2,3-c]pyridine-2-carboxylic acid methyl ester as a yellow solid (1.09 g, 4.2 mmol) which was used in subsequent reactions without further purification; ¹H NMR (d₆-DMSO, 400 MHz): δ 8.80 (1H, s), 8.32 (1H, s), 7.07 (1H, s), and 3.92 (3H, s); RP-HPLC (Hypersil C18, 5 μm, 100 Å, 15 cm; 5%-95% acetonitrile—0.05 M ammonium acetate over 15 min, 1 mL/min), Rₜ=9.19 min; m/z: (M+H)⁺ 256.2.

### General Procedure BB: Nucleophilic displacement with an amine

A methyl ester or trichloromethyloxadiazole (preferably 1 equivalent) and a nitrogen source (anhydrous ammonia (in MeOH or EtOH), hydrazine or an aliphatic amine) (100-300 equivalents, preferably 300 equivalents) is heated in a Parr mini-reactor at about 20-110° C. (preferably about 80° C.) for about 1-48 hours (preferably for about 12 hours). The mixture is allowed to cool to ambient temperature and the solvents are removed under reduced pressure to afford the product, which can be further purified by crystallization or chromatography.

Illustration of General Procedure BB

### Preparation #28 Preparation of 4-(biphenyl-4-ylamino)-1H-pyrrolo[2,3-c]pyridine-2-carboxylic acid amide

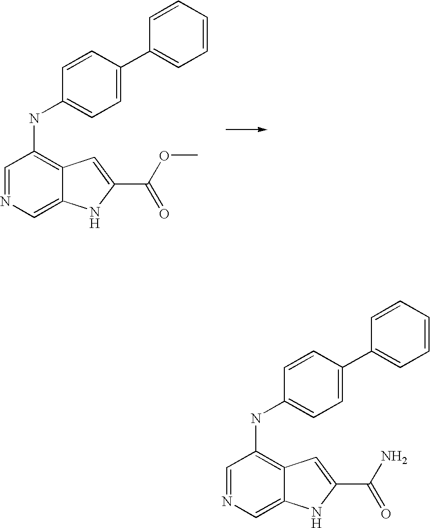

4-(Biphenyl-4-ylamino)-1H-pyrrolo[2,3-c]pyridine-2-carboxylic acid methyl ester (0.100 g) and anhydrous ammonia (7N in MeOH, 10 mL) was heated in a Parr mini-reactor at about 80° C. for about 24 hours. The mixture was allowed to cool to ambient temperature and the solvents were removed under reduced pressure. The residue was purified using preparative RP-HPLC (Table 1, Method k) to afford 4-(biphenyl-4-ylamino)-]H-pyrrolo[2,3-c]pyridine-2-carboxylic acid amide; ¹H NMR (d₆-DMSO, 400 MHz): δ 12.0 (s, 1H), 8.46 (s, 1H), 8.44 (s, 1H), 8.12 (s, 1H), 8.04 (s, 1H), 7.62 (m, 2H), 7.55 (m, 3H), 7.41 (m, 2H), 7.27 (m, 1H), and 7.08 (m, 3H); m/z: (M+H)⁺ 329.1

### General Procedure CC: Formation of thieno[2,3-c]pyridine-2-carboxylic acid methoxymethyl-amides from the corresponding carboxylic acids

A thieno[2,3-c]pyridine-2 carboxylic acid (preferably 1 equivalent) is dissolved in a suitable organic solvent (for example, DCM) and treated with an excess of oxalyl chloride and a catalytic amount of DMF. The mixture is stirred at ambient temperature for 1-12 hours (preferably about 4 hours). The solvents are removed under reduced pressure and the residue is dried in vacuo. The residue is dissolved in a suitable organic solvent (for example, DCM, DMF, NMP or THF, preferably DCM) and N,O-dimethyl hydroxylamine hydrochloride (1-3 equivalents, preferably 2.6 equivalents) and a tertiary amine base (3-10 equivalents, preferably 6.5 equivalents) are added. The reaction is stirred at room temperature for about 1-12 hours (preferably about 1 hour). The solvents are removed under reduced pressure and the product can be further purified by crystallization or chromatography.

Illustration of General Procedure CC

### Preparation #29: 4-(Biphenyl-4-ylamino)-thieno[2,3-c]pyridine-2-carboxylic acid methoxy-methyl-amide

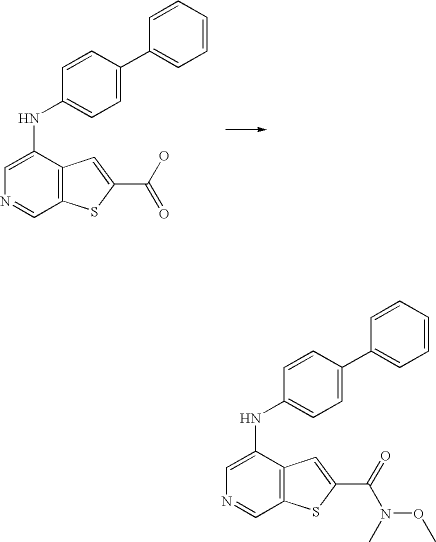

4-(Biphenyl-4-ylamino)-thieno[2,3-c]pyridine-2 carboxylic acid (prepared using general procedures A, B, I, and E) (0.070 g, 0.15 mmol) was dissolved in DCM (1.0 mL) and treated with oxalyl chloride (0.333 mL, 3.82 mmol) and a catalytic amount of DMF (0.005 mL). The mixture was stirred for about 4 hours at room temperature and the solvents were removed under reduced pressure. The residue was dissolved in DMF (1.0 mL) and N,O-dimethyl hydroxylamine hydrochloride (0.039 g, 0.40 mmol) and TEA (0.139 mL, 1.0 mmol) were added and the reaction mixture was stirred at room temperature for about 1 hour. Purification by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) afforded 4-(biphenyl-4-ylamino)-thieno[2,3-c]pyridine-2-carboxylic acid methoxy-methyl-amide as a yellow solid (0.033 g, 0.088 mmol); RP-HPLC (Table 1, Method i) Rₜ 3.24 min; m/z: (M+H)⁺ 390.1.

### General Procedure DD: Reduction with hydride

A hydride source (lithium aluminum hydride, sodium hydride or L-selectride) (1-2 equivalents, preferably 2 equivalents) is dissolved in an anhydrous organic solvent (MeOH or THF, preferably THF) and a solution of an ester, amide, aldehyde or nitrile (preferably 1 equivalent) in an anhydrous organic solvent (preferably THF) is added dropwise at about −78 to 0° C. The reaction mixture is warmed to ambient temperature and stirred for about 0.5-60 hours (preferably about 0.5 hours). The excess reagent is decomposed with the addition of dilute aqueous acid (preferably HCl) then partitioned between an aqueous inorganic base solution (preferably KOH) and an organic solvent (preferably DCM) separated, dried over dessicant (preferably magnesium sulfate) and filtered; or alternatively by the addition of Celite®, wet with a saturated aqueous potassium carbonate solution, allowed to stir at room temperature for about 1-24 hours (preferably about 2 hours) after which the celite is removed by filtration; or alternatively by the addition of saturated aqueous ammonium chloride solution, partitioning between an organic solvent (preferably DCM) and brine, drying over dessicant (preferably magnesium chloride) and filtering; or alternatively by the addition of sodium sulfate decahydrate until clear, followed by filtration. The crude product can be further purified by crystallization or chromatography.

Illustration of General Procedure DD

### Preparation #30: 4-(Biphenyl-4-yloxy)-thieno[2,3-c]pyridine-2-carbaldehyde

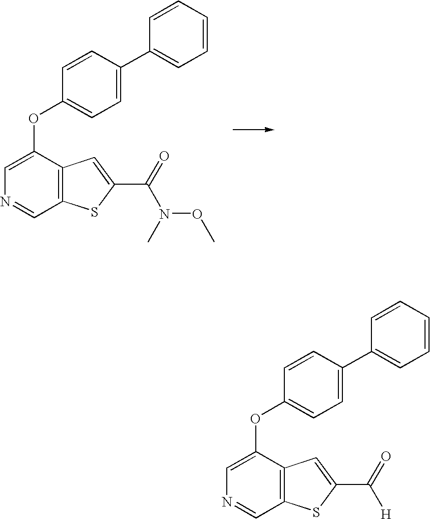

Lithium aluminum hydride (0.020 g, 0.52 mmol) was suspended in THF (1.0 mL) and a solution of 4-(biphenyl-4-yloxy)-thieno[2,3-c]pyridine-2-carboxylic acid methoxymethyl-amide (prepared using general procedures D, E, and CC) (0.100 g, 0.256 mmol) in THF (1.5 mL) was added dropwise at about 0° C. The reaction mixture was allowed to stir at room temperature for about 30 minutes and then Celite (0.200 g, wet with a saturated potassium carbonate solution (0.10 mL)) was added and the mixture was allowed to stir at room temperature for about 2 hours. The celite was removed by filtration and the product was further purified by column chromatography on silica gel using 5% EtOAc:DCM as the eluant. The fractions containing the product were combined and concentrated in vacuo to yield 4-(biphenyl-4-yloxy)-thieno[2,3-c]pyridine-2-carbaldehyde as an off-white solid (0.028 g, 0.084 mmol); RP-HPLC (Table 1, Method i) Rₜ=3.73 min; m/z: (M+H)⁺ 332.2.

### General Procedure EE: Preparation of thieno[2,3-c]pyridine-2-acetic acids from the corresponding thieno[2,3-c]pyridine-2-carbaldehyde

A thieno[2,3-c]pyridine-2-carbaldehyde (preferably 1 equivalent) and [(diethoxyphosphoryl)-dimethylamino-methyl]-phosphonic acid diethyl ester (preferably 1.3 equivalents) are dissolved in an organic solvent (for example, 1,4-dioxane or THF, preferably 1,4-dioxane) and the mixture is cooled at about 0° C. Sodium hydride is added and the reaction is allowed to stir at room temperature for about 0.5-4 hours (preferably about 0.5 hours). Aqueous HCl (6 N, 1 mL) is added and the mixture is heated at reflux for about 0.5-4 hours (preferably about 1 hour) until the intermediate is completely decomposed as judged by HPLC analysis. The reaction mixture is cooled to ambient temperature and the solvents are removed in vacuo. The product may be further purified by crystallization or chromatography.

Illustration of General Procedure EE

### Preparation #31: [4-(Biphenyl-4-yloxy)-thieno[2,3-c]pyridin-2-yl]-acetic acid

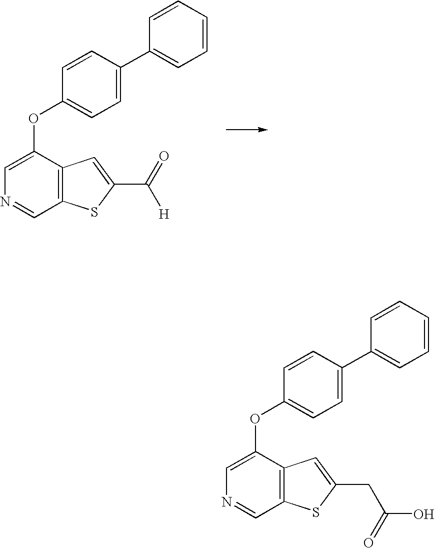

[4-(Biphenyl-4-yloxy)-thieno[2,3-c]pyridine-2-carbaldehyde (prepared using general procedures A, D, CC, and DD) (0.100 g, 0.300 mmol) and [(diethoxy-phosphoryl)-dimethylamino-methyl]-phosphonic acid diethyl ester (0.132 g, 0.400 mmol) are dissolved in 1,4-dioxane (3.0 mL) and the mixture was cooled to about 0° C. The mixture was treated with 60% NaH/mineral oil portions (4×0.016 g, 0.40 mmol) and the reaction was stirred at room temperature for about 0.5 hours. Aqueous HCl (4M, 1.0 mL) was added and the reaction mixture was heated at reflux for about 1 hour. The solvents were removed under reduced pressure and the residue was purified by RP-HPLC (20%-100% acetonitrile/0.05M aqueous ammonium acetate, buffered to pH 4.5, over 25 mnin at 15 mL/min; λ=254 nm; Hypersil C18, 100 Å, 8 μm, 250×21.2 mm column) to give [4-(biphenyl-4-yloxy)-thieno[2,3-c]pyridin-2-yl]-acetic acid as an off-white solid (0.033 g, 0.090 mmol); RP-HPLC (Table 1, Method i) Rₜ=1.66 min; m/z: (M+H)⁺ 362.2.

### General Procedure FF: Condensation of succinic anhydride with 2-amino-thieno[2,3-c]pyridines

A 2-amino-thieno[2,3-c]pyridine (preferably 1 equivalent) is dissolved in a suitable organic solvent (for example, NMP, DMF, or THF, preferably DMF) and treated with succinic anhydride (2-3 equivalents, preferably 2.4 equivalents). The mixture is heated at about 70-150° C. (preferably at about 90° C.) for 10-24 hours (preferably for about 14 hours). The product can be further purified by crystallization or chromatography.

Illustration of General Procedure FF

### Preparation #32: 1-[4-(Biphenyl-4-yloxy)-thieno[2,3-c]pyridin-2-yl]-pyrrolidine-2,5-dione

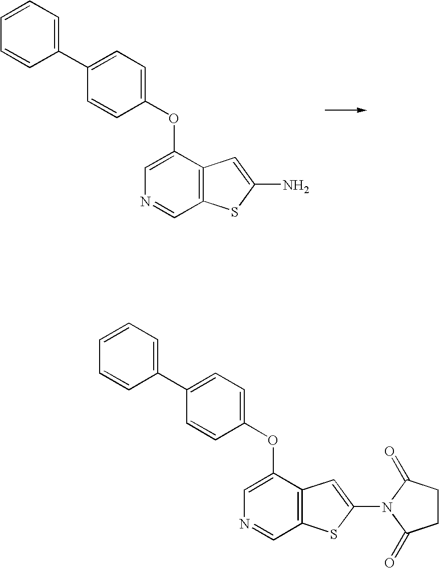

4-(Biphenyl-4-yloxy)-thieno[2,3-c]pyridin-2-ylamine (0.050 g, 0.16 mmol) was diluted with DMF (2.0 mL) and treated with succinic anhydride (0.032 g, 0.32 mmol) and the reaction was stirred at about 90° C. for about 16 hours. The product was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column). Product fractions were combined and concentrated in vacuo to afford an aqueous suspension. The product was collected by filtration and dried in vacuo to give 1-[4-(biphenyl-4-yloxy)-thieno[2,3-c]pyridin-2-yl]-pyrrolidine-2,5-dione as an off white solid (0.013 g, 0.034 mmol); RP-HPLC (Table 1, Method I) Rₜ=2.95 min; m/z: (M+H)⁺ 401.

### General procedure GG: Acid-catalysed t-butyloxycarbonyl deprotection and subsequent saponification

A pyrrolo[2,3-c]pyridine-1,2-dicarboxylic acid 1-tert-butyl ester 2-methyl ester (preferably 1 equivalent) in an anhydrous solvent (for example, EtOAc or DCM, preferably DCM) is stirred at about 0 to 20° C. (preferably at about 0° C.) for 0 to 10 minutes (preferably for about 5 minutes). An acidic solution (for example, hydrochloric acid or trifluoroacetic acid) (preferably trifluoroacetic acid) (10-100 equivalents, preferably 10 equivalents) is added dropwise. The solution is stirred for about 1-10 minutes (preferably for about 10 minutes), the ice bath is removed, and the solution is stirred for 1 to 12 hours (preferably for about 7 hours) at ambient temperature. The solvent is removed in vacuo to give a solid that may be used in subsequent reactions without further purification or purified by crystallization or chromatography. The solid is dissolved in an organic solvent (preferably MeOH) and an aqueous base (for example, lithium hydroxide, potassium hydroxide, or sodium hydroxide, preferably potassium hydroxide) (10-100 equivalents, preferably 50 equivalents) was added. The resulting solution is stirred for 1 to 24 hours (preferably for about 16 hours) at about 20-60° C. (preferably at about 22° C.). The solvent is removed in vacuo and the residue is acidified with 3N aqueous HCl to reach a pH from 1 to 4.5. The precipitate is filtered, washed with of water, and dried in vacuo. The product can be further purified by chromatography or crystallization.

Illustration of General Procedure GG

### Preparation #33: 4-(Biphenyl-4-ylamino)-1H-pyrrolo[2,3-c]pyridine-2-carboxylic acid methyl ester

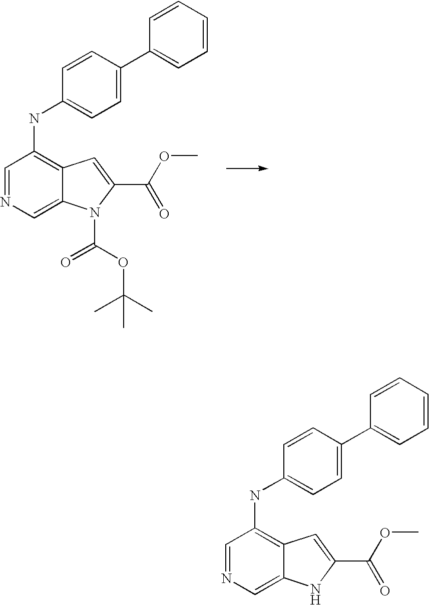

A mixture of 4-(biphenyl-4-ylamino)-pyrrolo[2,3-c]pyridine-1,2-dicarboxylic acid 1-tert-butyl ester 2-methyl ester (prepared using general procedures Z, AA, and I) (1.55 g, 3.49 mmol) in anhydrous DCM (10 mL) was stirred at about 0° C. for about 5 minutes and then a 3:1 mixture of DCM:TFA (10 equivalents) was added dropwise. The reaction mixture was stirred for about 10 minutes, the ice bath was removed, and the reaction mixture was stirred for about 7 hours at room temperature. The solvent was removed in vacuo and the residue was dissolved in THF (50 mL) and triethylamine (0.48 mL, 3.5 mmol). The solution was filtered through a plug of silica gel and concentrated in vacuo to afford 4-(biphenyl-4-ylamino)-1H-pyrrolo[2,3-c]pyridine-2-carboxylic acid methyl ester as a yellow solid (1.10 g, 3.20 mmol) that was used in the subsequent reactions without further purification; RP-HPLC (Table 1, Method n): Rₜ=3.88 min; m/z: (M+H)⁺ 344.1.

### General Procedure HH: Base-promoted nucleophilic substitution

An electrophile (alkylhalide, acyl halide, isocyante, anhydride, haloformate, ester preferably 1 equivalent) and a nucleophile (amino or hydroxyl containing substrate or latent enolate) (preferably 1 equivalent) are dissolved in an anhydrous solvent (THF, DMF, pyridine or DCM, preferably DMF) at −78□ C. to ambient temperature. If necessary, a base is added (for example, sodium hydride, triethylamine, diisopropylethylamine, cesium carbonate, potassium t-butoxide, or sodium carbonate preferably cesium carbonate, 1-4 equivalents, preferable 1.2 equivalent) and the solution is warmed to about ambient temperature −100° C., as necessary, for 2-72 hours (preferably 18 hours). The solvent is removed in vacuo; or alternatively the reaction is partitioned between an organic solvent (preferably DCM) and an aqueous inorganic base (preferably sodium bicarbonate), separated, washed with brine and dried over dessicant (magnesium or sodium sulfate, preferably sodium sulfate) and concentrated in vacuo; to afford the product, which can be further purified by chromatography or crystallization.

Illustration of General Procedure HH

### Preparation #34: 4-[4-(2-Pyrazol-1-yl-acetylamino)-phenyl]-thieno[2,3-c]pyridine-2-carboxylic acid amide (Example#462)

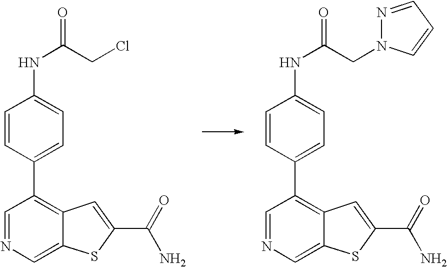

A solution of 4-[4-(2-Chloro-acetylamino)-phenyl]-thieno[2,3-c]pyridine-2-carboxylic acid amide (0.172 g, 0.500 mmol), 1H-Pyrazole (0.034 g, 0.50 mmol), and Cesium Carbonate (0.192 g, 0.600 mmol) in Dimethylformamide (5 mL) was allowed to stir for 18 hours at room temperature, and for an additional 15 minutes at 100° C. in the microwace. The reaction mixture was cooled to ambient temperature, and the solvent was removed in vacuo. The crude solid was taken up in DMSO and purified via preparative RP-HPLC (5% to 55% acetonitrile/0.05M aqueous ammonium acetate, buffered to pH 4.5, over 25 min, then 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to afford 4-[4-(2-Pyrazol-1-yl-acetylamino)-phenyl]-thieno[2,3-c]pyridine-2-carboxylic acid amide (0.027 g, 0.070 mmol) as a yellow clumpy solid; RP-HPLC (Table 1, Method a) Rₜ 7.53 min; m/z: (M+H)⁺ 378

Other compounds obtained using general procedure D are shown in Table 2.

TABLE 2
Examples synthesized using general procedure D

| | | | HPLC Rₜ | |
|---|---|---|---|---|
| Precursor Phenol | Product | Ex. # | (Method) | m/z |
| 4-Iodophenol | 4-(4-Iodo- | 1 | 2.54 min | 397(M+H)⁺ |
| | phenoxy)- | | (i) | |
| | thieno[2,3- | | | |
| | c]pyridine-2- | | | |
| | carboxylic acid | | | |
| | amide | | | |
| 4-Iodophenol | 4-(4-Iodo- | 2 | 2.32 min | 412(M+H)⁺ |
| | phenoxy)- | | (i) | |
| | thieno[2,3- | | | |
| | c]pyridine-2- | | | |
| | carboxylic acid N- | | | |
| | methyl-hydrazide | | | |
| Biphenyl-4-ol | 4-(Biphenyl-4- | 3 | 0.69 min | 348(M+H)⁺ |
| | yloxy)-thieno[2,3- | | (i) | |
| | c]pyridine-2- | | | |
| | carboxylic acid | | | |
| Biphenyl-4-ol | 4-(Biphenyl-4- | 4 | 2.11 min | 347(M+H)⁺ |
| | yloxy)-thieno[2,3- | | (i) | |
| | c]pyridine-2- | | | |
| | carboxylic acid | | | |
| | amide | | | |

Other compounds obtained using general procedure E are shown in Table 3.

TABLE 3
Examples synthesized using general procedure E

| | | | | m/z or ¹H |
|---|---|---|---|---|
| | | | | NMR (d₆ |
| | | | HPLC Rₜ | DMSO, |
| Precursor ester | Product | Ex. # | (Method) | 400MHz) |
| 4-(4-Iodo- | 4-(4-Iodo-phenoxy)- | 5 | 1.47 min | 396(M−H)⁻ |
| phenoxy)thieno[2,3- | thieno[2,3-c]pyridine-2- | | (i) | |
| c]pyridine-2- | carboxylic acid | | | |
| carboxylic acid | | | | |
| methyl ester (D) | | | | |
| 4-Fluoro-thieno[2,3- | 4-Fluoro-thieno[2,3- | 6 | 2.24 min | 7.51(s, |
| c]pyridine-2- | c]pyridine-2-carboxylic | | (i) | 1H), |
| carboxylic acid tert- | acid | | | 8.35-8.37(d, |
| butyl ester (A, B) | | | | 1H), |
| | | | | 8.97-8.99(1H, |
| | | | | 1H) |
| 4-(Biphenyl-4- | 4-(Biphenyl-4-ylamino)- | 7 | 2.82 min | 330(M+H)⁺ |
| ylamino)-1H- | 1H-pyrrolo[2,3- | | (n) | |
| pyrrolo[2,3-c]- | c]pyridine-2-carboxylic | | | |
| pyridine-2-carboxylic | acid | | | |
| acid methyl ester-TFA | | | | |
| salt (Z, AA, I) | | | | |
| 4-(Biphenyl-4- | 4-(Biphenyl-4-yloxy)-1H- | 8 | 1.58 min | 331(M+H)⁺ |
| yloxy)-1H- | pyrrolo[2,3-c]pyridine-2- | | (i) | |
| pyrrolo[2,3- | carboxylic acid | | | |
| c]pyridine-2- | | | | |
| carboxylic acid | | | | |
| methyl ester (Z, AA, | | | | |
| T) | | | | |
| 4-(3-Chloro-phenyl)- | 4-(3-Chloro-phenyl)- | 9 | 6.14 min | 287.8, |
| thieno[2,3-c]pyridine- | thieno[2,3-c]pyridine-2- | | (b) | 289.8(M−H)⁻ |
| 2-carboxylic acid | carboxylic acid | | | |
| methyl ester | | | | |
| (A, B, J) | | | | |
| 7-(Biphenyl-4- | 7-(Biphenyl-4-ylamino)- | 10 | 2.17 min | 347(M |
| ylamino)-thieno[2,3- | thieno[2,3-c]pyridine-2- | | (i) | H)⁺ |
| c]pyridine-2- | carboxylic acid | | | |
| carboxylic acid | | | | |
| methyl ester | | | | |
| (A, B, OO, AAA, I, | | | | |
| PP) | | | | |
| 4-(4-tert-Butyl- | 4-(4-tert-Butyl- | 11 | 6.81 min | 327(M+H)⁺ |
| phenylamino)- | phenylamino)-thieno[2,3- | | (b) | (i) |
| thieno[2,3-c]pyridine- | c]pyridine-2-carboxylic | | | |
| 2-carboxylic acid | acid | | | |
| methyl ester | | | | |
| (A, B, I) | | | | |
| 7-(Biphenyl-4- | 7-(Biphenyl-4-ylamino)- | 12 | | 347(M+H)⁺; |
| ylamino)-thieno[2,3- | thieno[2,3-c]pyridine-2- | | | 13.9(bs, |
| c]pyridine-2- | carboxylic acid | | | 1H), |
| carboxylic acid | | | | 9.19(s, |
| methyl ester (A, B, I) | | | | 1H), |
| | | | | 8.11-8.15(d, |
| | | | | 1H), |
| | | | | 8.07(s, |
| | | | | 1H), |
| | | | | 7.89-7.95(m, |
| | | | | 2H), |
| | | | | 7.61-7.69(m, |
| | | | | 4H), |
| | | | | 7.41-7.48(m, |
| | | | | 2H), |
| | | | | 7.37-7.41(d, |
| | | | | 1H), |
| | | | | and 7.28-7.35(m, |
| | | | | 1H) |
| 3′-(7-Amino-2- | 3′-(7-Amino-2- | 12A | 4.48 min | 390.33(M+H)⁺ |
| carbamoyl- | carbamoyl-thieno[2,3- | | (q) | |
| thieno[2,3- | c]pyridin-4-yl)- | | | |
| c]pyridin-4-yl)- | biphenyl-4-carboxylic | | | |
| biphenyl-4- | acid | | | |
| carboxylic acid | | | | |
| methyl ester | | | | |
| (A, B, OO, J, AAA, I, BB, | | | | |
| II) | | | | |
| 7-Amino-4-(3′,4′- | 7-Amino-4-(3′,4′- | 12B | 4.60 min | 407.22(M+H)⁺ |
| dimethoxy- | dimethoxy-biphenyl-3- | | (q) | |
| biphenyl-3-yl)- | yl)-thieno[2,3- | | | |
| thieno[2,3- | c]pyridine-2-carboxylic | | | |
| c]pyridine-2- | acid | | | |
| carboxylic acid | | | | |
| methyl ester | | | | |
| (A, B, OO, J, AAA, I, II) | | | | |
| 7-Amino-4-(4′- | 7-Amino-4-(4′- | 12C | 3.55 min | 390.3(M+H)⁺ |
| carbamoyl- | carbamoyl-biphenyl-3- | | (q) | |
| biphenyl-3-yl)- | yl)-thieno[2,3- | | | |
| thieno[2,3- | c]pyridine-2-carboxylic | | | |
| c]pyridine-2- | acid | | | |
| carboxylic acid | | | | |
| methyl ester | | | | |
| (A, B, OO, J, AAA, I, II) | | | | |
| 7-Amino-4-(4′- | 7-Amino-4-(4′- | 12D | 4.79 min | 377.22(M+H)⁺ |
| methoxy-biphenyl- | methoxy-biphenyl-3- | | (q) | |
| 3-yl)-thieno[2,3- | yl)-thieno[2,3- | | | |
| c]pyridine-2- | c]pyridine-2-carboxylic | | | |
| carboxylic acid | acid | | | |
| methyl ester | | | | |
| (A, B, OO, J, AAA, I, II) | | | | |
| 7-Amino-4-(3- | 7-Amino-4-(3- | 12E | 6.02 min | 403.3(M+H)⁺ |
| benzo[b]thiophen-3- | benzo[b]thiophen-3-yl- | | (q) | |
| yl-phenyl)- | phenyl)-thieno[2,3- | | | |
| thieno[2,3- | c]pyridine-2-carboxylic | | | |
| c]pyridine-2- | acid | | | |
| carboxylic acid | | | | |
| methyl ester | | | | |
| (A, B, OO, J, AAA, I, II) | | | | |
| 7-Amino-4-(4′- | 7-Amino-4-(4′-ethyl- | 12F | 5.23 min | 375.3(M+H)⁺ |
| ethyl-biphenyl-3- | biphenyl-3-yl)- | | (q) | |
| yl)-thieno[2,3- | thieno[2,3-c]pyridine-2- | | | |
| c]pyridine-2- | carboxylic acid | | | |
| carboxylic acid | | | | |
| methyl ester | | | | |
| (A, B, OO, J, AAA, I, II) | | | | |
| 7-Amino-4-(3- | 7-Amino-4-(3-pyridin- | 12G | 3.92 min | 348.2(M+H)⁺ |
| pyridin-4-yl- | 4-yl-phenyl)-thieno[2,3- | | (q) | |
| phenyl)-thieno[2,3- | c]pyridine-2-carboxylic | | | |
| c]pyridine-2- | acid | | | |
| carboxylic acid | | | | |
| methyl ester | | | | |
| (A, B, OO, J, AAA, I, II) | | | | |
| 7-Amino-4-(3′- | 7-Amino-4-(3′- | 12H | 4.37 min | 425.2(M+H)⁺ |
| methanesulfonyl- | methanesulfonyl- | | (q) | |
| biphenyl-3-yl)- | biphenyl-3-yl)- | | | |
| thieno[2,3- | thieno[2,3-c]pyridine-2- | | | |
| c]pyridine-2- | carboxylic acid | | | |
| carboxylic acid | | | | |
| methyl ester | | | | |
| (A, B, OO, J, AAA, I, II) | | | | |
| 4-[2,2′]Bithiophenyl- | 4-[2,2′]Bithiophenyl-5-yl- | 12I | 6.65 min | 341.8(M−H)⁻ |
| 5-yl-thieno[2,3- | thieno[2,3-c]pyridine-2- | | (b) | |
| c]pyridine-2- | carboxylic acid | | | |
| carboxylic acid | | | | |
| methyl ester (A, B, J) | | | | |
| 5-(Biphenyl-3- | 5-(Biphenyl-3-ylamino)- | 12J | 1.28 min | 347(M+H)⁺ |
| ylamino)-thieno[2,3- | thieno[2,3-c]pyridine-2- | | (a) | 345(M−H)⁻ |
| c]pyridine-2- | carboxylic acid | | | |
| carboxylic acid | | | | |
| methyl ester (KC-3, | | | | |
| KC-4) | | | | |

Other compounds obtained using general procedure F are shown in Table 4.

TABLE 4
Examples synthesized using general procedure F

| | | | HPLC Rₜ | |
|---|---|---|---|---|
| Precursor amide | Product | Ex. # | (Method) | m/z |
| 4-(4-Iodo-phenoxy)- | 4-(4-Iodo- | 13 | 3.61 min (i) | 379(M+H)⁺ |
| thieno[2,3-c]pyridine- | phenoxy)- | | | |
| 2-carboxylic acid | thieno[2,3- | | | |
| amide (D) | c]pyridine-2- | | | |
| | carbonitrile | | | |
| 4-(Biphenyl-4- | 4-(Biphenyl-4- | 14 | 3.34 min (i) | 329(M+H)⁺ |
| yloxy)-thieno[2,3- | yloxy)-thieno[2,3- | | | |
| c]pyridine-2- | c]pyridine-2- | | | |
| carboxylic acid amide | carbonitrile | | | |
| (D) | | | | |
| 4-(Biphenyl-4- | 4-(Biphenyl-4- | 15 | 5.70 min (j) | 360(M−H)⁻ |
| ylamino)-7-chloro- | ylamino)-7-chloro- | | | |
| thieno[2,3-c]pyridine- | thieno[2,3- | | | |
| 2-carboxylic acid | c]pyridine-2- | | | |
| amide | carbonitrile | | | |
| 4-[3-(3-m-Tolyl- | 1-[3-(2-Cyano- | 16 | 11.28 (a) | 385(M+H)⁺ |
| ureido)-phenyl]- | thieno[2,3-c]pyridin- | | | |
| thieno[2,3-c]pyridine- | 4-yl)-phenyl]-3-m- | | | |
| 2-carboxylic acid | tolyl-urea | | | |
| amide | | | | |
| (A, C, J, N) | | | | |
| 4-Biphenyl-3-yl- | 4-Biphenyl-3-yl- | 16A | 12.83 min (a) | 313.4(M+H)⁺ |
| thieno[2,3-c]pyridine- | thieno[2,3- | | | |
| 2-carboxylic acid | c]pyridine-2- | | | |
| amide | carbonitrile | | | |
| (A, C, J) | | | | |

Other compounds obtained using general procedure G are shown in Table 5.

TABLE 5
Examples synthesized using general procedure G

| | | | HPLC Rₜ | |
|---|---|---|---|---|
| Tetrazole precursor | Product | Ex # | (Method) | m/z |
| 4-(3,5-Dimethyl- | (3,5-Dimethyl-biphenyl- | 17 | 8.61 min | 399.01(M+H)⁺ |
| biphenyl-4-ylamino)- | 4-yl)-[2-(2H-tetrazol-5- | | (a) | |
| thieno[2,3- | yl)-thieno[2,3-c]pyridin- | | | |
| c]pyridine-2- | 4-yl]-amine | | | |
| carbonitrile (Y, I | | | | |
| (A, C, F)) | | | | |
| 4-(3-Fluoro- | (3-Fluoro-biphenyl-4-yl)- | 18 | 8.30 min | 388.99(M+H)⁺ |
| biphenyl-4-ylamino)- | [2-(2H-tetrazol-5-yl)- | | (a) | |
| thieno[2,3- | thieno[2,3-c]pyridin-4- | | | |
| c]pyridine-2- | yl]-amine | | | |
| carbonitrile (Y, I | | | | |
| (A, C, F)) | | | | |
| 4-(3-Chloro- | (3-Chloro-biphenyl-4-yl)- | 19 | 8.54 min | 404.97(M+H)⁺ |
| biphenyl-4-ylamino)- | [2-(2H-tetrazol-5-yl)- | | (a) | |
| thieno[2,3- | thieno[2,3-c]pyridin-4- | | | |
| c]pyridine-2- | yl]-amine | | | |
| carbonitrile (Y, I | | | | |
| (A, C, F)) | | | | |
| 4-(3- | [2-(2H-Tetrazol-5-yl)- | 20 | 8.78 min | 439.0(M+H)⁺ |
| Trifluoromethyl- | thieno[2,3-c]pyridin-4- | | (a) | |
| biphenyl-4-ylamino)- | yl]-(3-trifluoromethyl- | | | |
| thieno[2,3- | biphenyl-4-yl)-amine | | | |
| c]pyridine-2- | | | | |
| carbonitrile (Y, I | | | | |
| (A, C, F)) | | | | |
| 4-(2- | [2-(2H-Tetrazol-5-yl)- | 21 | 8.83 min | 438.97(M+H)⁺ |
| Trifluoromethyl- | thieno[2,3-c]pyridin-4- | | (a) | |
| biphenyl-4-ylamino)- | yl]-(2-trifluoromethyl- | | | |
| thieno[2,3- | biphenyl-4-yl)-amine | | | |
| c]pyridine-2- | | | | |
| carbonitrile (Y, I | | | | |
| (A, C, F)) | | | | |
| 4-(3-Methyl- | (3-Methyl-biphenyl-4-yl)- | 22 | 8.40 min | 385.0(M+H)⁺ |
| biphenyl-4-ylamino)- | [2-(2H-tetrazol-5-yl)- | | (a) | |
| thieno[2,3- | thieno[2,3-c]pyridin-4- | | | |
| c]pyridine-2- | yl]-amine | | | |
| carbonitrile (Y, I | | | | |
| (A, C, F)) | | | | |
| 4-Biphenyl-3-yl- | 4-Biphenyl-3-yl-2-(2H- | 22A | 8.32 min | 356.0(M+H)⁺ |
| thieno[2,3- | tetrazol-5-yl)-thieno[2,3- | | (a) | |
| c]pyridine-2- | c]pyridine | | | |
| carbonitrile | | | | |
| (A, C, J, F, G) | | | | |

Other compounds obtained using general procedure H are shown in Table 6.

TABLE 6
Examples synthesized using general procedure H

| | | | HPLC Rₜ | |
|---|---|---|---|---|
| Hydrolysis precursor | Product | Ex # | (Method) | m/z |
| 4-(2-Chloro-biphenyl- | 4-(2-Chloro-biphenyl-4- | 23 | 10.23 min | 380.2(M+H)⁺ |
| 4-ylamino)- | ylamino)-thieno[2,3- | | (a) | |
| thieno[2,3-c]pyridine- | c]pyridine-2-carboxylic | | | |
| 2-carbonitrile (Y, I | acid amide | | | |
| (A, C, F)) | | | | |
| 4-(3,5-Difluoro- | 4-(3,5-Difluoro- | 24 | 9.80 min (a) | 382.2(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid amide | | | |
| (A, C, F)) | | | | |
| 4-(3,5-Difluoro- | 4-(3,5-Difluoro- | 25 | 8.07 min (a) | 383.2(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid | | | |
| (A, C, F)) | | | | |
| 4-(3,5-Dimethyl- | 4-(3,5-Dimethyl- | 26 | 10.34 min | 374.3(M+H)⁺ |
| biphenyl-4-ylamino)- | biphenyl-4-ylamino)- | | (a) | |
| thieno[2,3-c]pyridine- | thieno[2,3-c]pyridine-2- | | | |
| 2-carbonitrile (Y, I | carboxylic acid amide | | | |
| (A, C, F)) | | | | |
| 4-(3,5-Dimethyl- | 4-(3,5-Dimethyl- | 27 | 8.32 min (a) | 375.3(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid | | | |
| (A, C, F)) | | | | |
| 4-(3-Fluoro-biphenyl- | 4-(3-Fluoro-biphenyl-4- | 28 | 9.86 min (a) | 364.2(M+H)⁺ |
| 4-ylamino)- | ylamino)-thieno[2,3- | | | |
| thieno[2,3-c]pyridine- | c]pyridine-2-carboxylic | | | |
| 2-carbonitrile (Y, I | acid amide | | | |
| (A, C, F)) | | | | |
| 4-(3-Fluoro-biphenyl- | 4-(3-Fluoro-biphenyl-4- | 29 | 8.03 min (a) | 365.2(M+H)⁺ |
| 4-ylamino)- | ylamino)-thieno[2,3- | | | |
| thieno[2,3-c]pyridine- | c]pyridine-2-carboxylic | | | |
| 2-carbonitrile (Y, I | acid | | | |
| (A, C, F)) | | | | |
| 4-(3-Chloro-biphenyl- | 4-(3-Chloro-biphenyl-4- | 30 | 10.36 min | 380.2(M+H)⁺ |
| 4-ylamino)- | ylamino)-thieno[2,3- | | (a) | |
| thieno[2,3-c]pyridine- | c]pyridine-2-carboxylic | | | |
| 2-carbonitrile (Y, I | acid amide | | | |
| (A, C, F)) | | | | |
| 4-(3-Chloro-biphenyl- | 4-(3-Chloro-biphenyl-4- | 31 | 8.28 min (a) | 381.2(M+H)⁺ |
| 4-ylamino)- | ylamino)-thieno[2,3- | | | |
| thieno[2,3-c]pyridine- | c]pyridine-2-carboxylic | | | |
| 2-carbonitrile (Y, I | acid | | | |
| (A, C, F)) | | | | |
| 4-(3-Trifluoromethyl- | 4-(3-Trifluoromethyl- | 32 | 10.62 min | 414.2(M+H)⁺ |
| biphenyl-4-ylamino)- | biphenyl-4-ylamino)- | | (a) | |
| thieno[2,3-c]pyridine- | thieno[2,3-c]pyridine-2- | | | |
| 2-carbonitrile (Y, I | carboxylic acid amide | | | |
| (A, C, F)) | | | | |
| 4-(3-Trifluoromethyl- | 4-(3-Trifluoromethyl- | 33 | 8.52 min (a) | 415.2(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid | | | |
| (A, C, F)) | | | | |
| 4-(2-Trifluoromethyl- | 4-(2-Trifluoromethyl- | 34 | 5.27 min (j) | 414.2(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid amide | | | |
| (A, C, F)) | | | | |
| 4-(2-Trifluoromethyl- | 4-(2-Trifluoromethyl- | 35 | 8.57 min (a) | 415.2(M+H)⁺ |
| biphenyl-4-ylamino)- | biphenyl-4-ylamino)- | | | |
| thieno[2,3-c]pyridine- | thieno[2,3-c]pyridine-2- | | | |
| 2-carbonitrile (Y, I | carboxylic acid | | | |
| (A, C, F)) | | | | |
| 4-(3-Methoxy- | 4-(3-Methoxy-biphenyl- | 36 | 9.90 min (a) | 376.2(M+H)⁺ |
| biphenyl-4-ylamino)- | 4-ylamino)-thieno[2,3- | | | |
| thieno[2,3-c]pyridine- | c]pyridine-2-carboxylic | | | |
| 2-carbonitrile (Y, I | acid amide | | | |
| (A, C, F)) | | | | |
| 4-(3-Methoxy- | 4-(3-Methoxy-biphenyl- | 37 | 7.98 min (a) | 377.2(M+H)⁺ |
| biphenyl-4-ylamino)- | 4-ylamino)-thieno[2,3- | | | |
| thieno[2,3-c]pyridine- | c]pyridine-2-carboxylic | | | |
| 2-carbonitrile (Y, I | acid | | | |
| (A, C, F)) | | | | |
| 4-(3-Chloro- | 4-(3-Chloro- | 38 | 7.15 min (a) | 305.2(M+H)⁺ |
| phenylamino)- | phenylamino)- | | | |
| thieno[2,3-c]pyridine- | thieno[2,3-c]pyridine-2- | | | |
| 2-carbonitrile (Y, I | carboxylic acid | | | |
| (A, C, F)) | | | | |
| 4-(2-Chloro-biphenyl- | 4-(2-Chloro-biphenyl-4- | 39 | 8.33 min (a) | 381.2(M+H)⁺ |
| 4-ylamino)- | ylamino)-thieno[2,3- | | | |
| thieno[2,3-c]pyridine- | c]pyridine-2-carboxylic | | | |
| 2-carbonitrile (Y, I | acid | | | |
| (A, C, F)) | | | | |
| 4-(3-Methyl- | 4-(3-Methyl-biphenyl-4- | 40 | 10.09 min | 360.3(M+H)⁺ |
| biphenyl-4-ylamino)- | ylamino)-thieno[2,3- | | (a) | |
| thieno[2,3-c]pyridine- | c]pyridine-2-carboxylic | | | |
| 2-carbonitrile (Y, I | acid amide | | | |
| (A, C, F)) | | | | |
| 4-(3-Methyl- | 4-(3-Methyl-biphenyl-4- | 41 | 8.16 min (a) | 361.2(M+H)⁺ |
| biphenyl-4-ylamino)- | ylamino)-thieno[2,3- | | | |
| thieno[2,3-c]pyridine- | c]pyridine-2-carboxylic | | | |
| 2-carbonitrile (Y, I | acid | | | |
| (A, C, F)) | | | | |
| 4-(1H-Pyrazol-4-yl)- | 4-(1H-Pyrazol-4-yl)- | 42 | 0.46 min (j) | 246.2(M+H)⁺ |
| thieno[2,3-c]pyridine- | thieno[2,3-c]pyridine-2- | | | |
| 2-carboxylic acid tert- | carboxylic acid | | | |
| butyl ester (A, B, J) | | | | |
| 4-(4-Hydroxy- | 4-(4-Hydroxy- | 43 | 6.44 min (a) | 286.1(M+H)⁺ |
| phenylamino)- | phenylamino)- | | | |
| thieno[2,3-c]pyridine- | thieno[2,3-c]pyridine-2- | | | |
| 2-carbonitrile (I | carboxylic acid amide | | | |
| (A, C, F)) | | | | |
| 4-(4-Amino- | 4-(4-Amino- | 44 | 6.04 min (a) | 285.2(M+H)⁺ |
| phenylamino)- | phenylamino)- | | | |
| thieno[2,3-c]pyridine- | thieno[2,3-c]pyridine-2- | | | |
| 2-carbonitrile (I | carboxylic acid amide | | | |
| (A, C, F)) | | | | |
| 4-(2-Cyano- | 4-(2-Carbamoyl- | 45 | 6.30 min (a) | 314.2(M+H)⁺ |
| thieno[2,3-c]pyridin- | thieno[2,3-c]pyridin-4- | | | |
| 4-ylamino)-benzoic | ylamino)-benzoic acid | | | |
| acid (I (A, C, F)) | | | | |
| 4-(4-Hydroxy- | 4-(4-Hydroxy- | 46 | 5.21 min (a) | 287.2(M+H)⁺ |
| phenylamino)- | phenylamino)- | | | |
| thieno[2,3-c]pyridine- | thieno[2,3-c]pyridine-2- | | | |
| 2-carbonitrile (I | carboxylic acid | | | |
| (A, C, F)) | | | | |
| 4-(Biphenyl-4- | 4-(Biphenyl-4-ylamino)- | 47 | 0.77 min (i) | 389(M−H)⁻ |
| ylamino)-7-cyano- | thieno[2,3-c]pyridine- | | | |
| thieno[2,3-c]pyridine- | 2,7-dicarboxylic acid | | | |
| 2-carboxylic acid | | | | |
| methyl ester (I (A, B)) | | | | |
| 4-(4-Amino- | 4-(4-Amino- | 48 | 4.86 min (a) | 286.2(M+H)⁺ |
| phenylamino)- | phenylamino)- | | | |
| thieno[2,3-c]pyridine- | thieno[2,3-c]pyridine-2- | | | |
| 2-carbonitrile (I | carboxylic acid | | | |
| (A, C, F)) | | | | |
| 4-Cyclohexylamino- | 4-Cyclohexylamino- | 49 | 8.39 (a) | 276.2(M+H)⁺ |
| thieno[2,3-c]pyridine- | thieno[2,3-c]pyridine- | | | |
| 2-carbonitrile (I | 2-carboxylic acid | | | |
| (A, C, F)) | amide | | | |
| 4-(4-Phenyl- | 4-(4-Phenyl- | 50 | 10.00, 10.19 | 352.2(M+H)⁺ |
| cyclohexylamino)- | cyclohexylamino)- | | (a) | |
| thieno | thieno | | | |
| [2,3-c]pyridine-2- | [2,3-c]pyridine-2- | | | |
| carbonitrile (I | carboxylic acid amide | | | |
| (A, C, F)) | | | | |
| 4-(1-Benzyl- | 4-(1-Benzyl-piperidin-4- | 51 | 6.65 (a) | 367.2(M+H)⁺ |
| piperidin-4-ylamino)- | ylamino)-thieno | | | |
| thieno | [2,3-c]pyridine-2- | | | |
| [2,3-c]pyridine-2- | carboxylic acid | | | |
| carbonitrile (I | amide | | | |
| (A, C, F)) | | | | |
| 4-(1-Benzyl- | 4-(1-Benzyl-piperidin-4- | 52 | 5.55 (a) | 368.2(M+H)⁺ |
| piperidin-4-ylamino)- | ylamino)-thieno | | | |
| thieno | [2,3-c]pyridine-2- | | | |
| [2,3-c]pyridine-2- | carboxylic acid | | | |
| carbonitrile (I | | | | |
| (A, C, F)) | | | | |
| (1R,5R)-6-(2-Cyano- | (1R,5R)-6-(2- | 53 | 8.27 (a) | 409.2(M+H)⁺ |
| thieno[2,3-c]pyridin- | Carbamoyl-thieno[2,3-c | | | |
| 4-yl)-3,6-diaza- | ]pyridin-4-yl)-3,6-diaza- | | | |
| bicyclo[3.2.0] | bicyclo[3.2.0 | | | |
| heptane-3-carboxylic | ]heptane-3- | | | |
| acid benzyl ester | carboxylic acid benzyl | | | |
| (I (A, C, F)) | ester | | | |
| 4-(4′-Formyl- | 4-(4′-Formyl-biphenyl- | 54 | 8.90 (a) | 374.2(M+H)⁺ |
| biphenyl-4-ylamino)- | 4-ylamino)-thieno | | | |
| thieno | [2,3-c]pyridine-2- | | | |
| [2,3-c]pyridine-2- | carboxylic acid | | | |
| carbonitrile (I | amide | | | |
| (A, C, F)) | | | | |
| 4-(2-Cyano- | 4-(2-Carbamoyl- | 55 | 8.48 (a) | 377.0(M+H)⁺ |
| thieno[2,3-c]pyridin- | thieno[2,3-c]pyridin- | | | |
| 4-ylamino)- | 4-ylamino)-piperidine- | | | |
| piperidine-1- | 1-carboxylic | | | |
| carboxylic | acid tert-butyl ester | | | |
| acid tert-butyl ester (I | | | | |
| (A, C, F)) | | | | |
| (R)-3-(2-Cyano- | (R)-3-(2-Carbamoyl- | 56 | 8.19 (a) | 363.0(M+H)⁺ |
| thieno[2,3-c]pyridin- | thieno[2,3-c]pyridin- | | | |
| 4-ylamino)- | 4-ylamino)- | | | |
| pyrrolidine-1-carboxylic | pyrrolidine-1-carboxylic | | | |
| acid tert-butyl | acid tert-butyl | | | |
| ester (I (A, C, F)) | ester | | | |
| 4-((3aS, 6aR)-5- | 4-((3aS, 6aR)-5-Benzyl- | 57 | 6.63 (a) | 379.2(M+H)⁺ |
| Benzyl-hexahydro- | hexahydro-pyrrolo | | | |
| pyrrolo | [3,4-c]pyrrol-2-yl)- | | | |
| [3,4-c]pyrrol-2- | thieno[2,3- | | | |
| yl)-thieno[2,3- | c]pyridine-2-carboxylic | | | |
| c]pyridine-2- | acid amide | | | |
| carbonitrile (I | | | | |
| (A, C, F)) | | | | |
| 4-(2-Benzyl- | 4-(2-Benzyl-octahydro- | 58 | 6.90 (a) | 393.2(M+H)⁺ |
| octahydro- | pyrrolo[3,4-c | | | |
| pyrrolo[3,4-c | ]pyridin-5-yl)- | | | |
| ]pyridin-5-yl)- | thieno[2,3-c]pyridine- | | | |
| thieno[2,3-c]pyridine- | 2-carboxylic acid | | | |
| 2-carbonitrile (I | amide | | | |
| (A, C, F)) | | | | |
| 4-(2-Benzyl-1-oxo- | 4-(2-Benzyl-1-oxo-2,8- | 59 | 8.72 (a) | 421.3(M+H)⁺ |
| 2,8-diaza-spiro[4.5 | diaza-spiro[4.5 | | | |
| ]dec-8-yl)- | ]dec-8-yl)-thieno[2,3- | | | |
| thieno[2,3-c]pyridine- | c]pyridine- | | | |
| 2-carbonitrile (I | 2-carboxylic acid amide | | | |
| (A, C, F)) | | | | |
| 4-(1-Oxo-2-phenyl- | 4-(1-Oxo-2-phenyl-2,8- | 60 | 8.85 (a) | 407.3(M+H)⁺ |
| 2,8-diaza-spiro[4.5 | diaza-spiro[4.5 | | | |
| ]dec-8-yl)- | ]dec-8-yl)-thieno[2,3- | | | |
| thieno[2,3-c]pyridine- | c]pyridine- | | | |
| 2-carbonitrile (I | 2-carboxylic acid amide | | | |
| (A, C, F)) | | | | |
| (1R,4S)-5-(2-Cyano- | (1R,4S)-5-(2- | 61 | 8.12 (a) | 375.1(M+H)⁺ |
| thieno[2,3-c | Carbamoyl-thieno[2,3-c | | | |
| ]pyridin-4-yl)-2,5- | ]pyridin-4-yl)-2,5-diaza- | | | |
| diaza-bicyclo[2.2.1 | bicyclo[2.2.1 | | | |
| ]heptane-2- | ]heptane-2- | | | |
| carboxylic acid tert- | carboxylic acid tert- | | | |
| butyl ester (I (A, C, F)) | butyl ester | | | |

Other compounds obtained using general procedure I are shown in Table 7.

TABLE 7
Examples synthesized using general procedure I

| Arylhalide | | | | HPLC Rₜ | |
|---|---|---|---|---|---|
| precursor | Amine precursor | Product Name | Ex # | (Method) | m/z |
| 4-Bromo- | Biphenyl-4- | 4-(Biphenyl-4- | 62 | 9.76 min | 346 |
| thieno[2,3-c] | ylamine | ylamino)- | | (a) | (M + H)⁺ |
| pyridine-2- | | thieno[2,3-c] | | | |
| carboxylic acid | | pyridine-2- | | | |
| amide (A, C) | | carboxylic acid | | | |
| | | amide | | | |
| 4-Bromo- | 3-Phenoxy- | 4-(3-Phenoxy- | 63 | 9.65 min | 362 |
| thieno[2,3-c] | phenylamine | phenylamino)- | | (a) | (M + H)⁺ |
| pyridine-2- | | thieno[2, | | | |
| carboxylic acid | | 3-c]pyridine-2- | | | |
| amide (A, C) | | carboxylic acid | | | |
| | | amide | | | |
| 4-Bromo- | 4-Phenoxy- | 4-(4-Phenoxy- | 64 | 9.71 min | 362 |
| thieno[2,3-c] | phenylamine | phenylamino)- | | (a) | (M + H)⁺ |
| pyridine-2- | | thieno[2, | | | |
| carboxylic acid | | 3-c]pyridine-2- | | | |
| amide (A, C) | | carboxylic acid | | | |
| | | amide | | | |
| 4-Bromo- | 4-Bromo- | 4-(4-Bromo- | 65 | 8.9 min | 348/350 |
| thieno[2,3-c] | phenylamine | phenylamino)- | | (a) | (M + H)⁺ |
| pyridine-2- | | thieno[2,3-c] | | | |
| carboxylic acid | | pyridine-2- | | | |
| amide (A, C) | | carboxylic acid | | | |
| | | amide | | | |
| 4-Bromo- | Naphthalen-2- | 4-(Naphthalen-2- | 66 | 9.11 min | 320 |
| thieno[2,3-c] | ylamine | ylamino)- | | (a) | (M + H)⁺ |
| pyridine-2- | | thieno[2,3- | | | |
| carbonitrile (A, | | c]pyridine-2- | | | |
| C, F) | | carboxylic acid | | | |
| | | amide | | | |
| 4-Bromo- | Biphenyl-4- | 4-(Biphenyl-4- | 67 | 13.76 min | 403 |
| thieno[2,3-c] | ylamine | ylamino)- | | (a) | (M + H)⁺ |
| pyridine-2- | | thieno[2,3-c] | | | |
| carboxylic acid | | pyridine-2- | | | |
| tert-butyl ester | | carboxylic acid | | | |
| (A, B) | | tert-butyl | | | |
| | | ester | | | |
| 4-Bromo- | 4-Bromo- | 4-(4-Bromo- | 68 | 10.50 min | 405, 407 |
| thieno[2,3-c] | phenylamine | phenylamino)- | | (b) | (M + H)⁺ |
| pyridine-2- | | thieno[2,3-c] | | | |
| carboxylic acid | | pyridine-2- | | | |
| tert-butyl ester | | carboxylic acid | | | |
| (A, B) | | tert-butyl | | | |
| | | ester | | | |
| 4-Bromo- | 4-Thiophen-3-yl- | 4-(4-Thiophen-3- | 69 | 7.79 min | 353 |
| thieno[2,3-c] | phenylamine | yl-phenylamino)- | | (a) | (M + H)⁺ |
| pyridine-2- | | thieno | | | |
| carboxylic acid | | [2,3-c] | | | |
| tert-butyl ester | | pyridine-2- | | | |
| (A, B) | | carboxylic acid | | | |
| 4-Bromo- | Naphthalen-2- | 4-(Naphthalen-2- | 70 | 7.45 min | 321 |
| thieno[2,3-c] | ylamine | ylamino)- | | (a) | (M + H)⁺ |
| pyridine-2- | | thieno[2,3-c] | | | |
| carbonitrile (A, | | pyridine-2- | | | |
| C, F) | | carboxylic acid | | | |
| 4-Bromo- | 4-Pyridin-4-yl- | 4-(4-Pyridin-4-yl- | 71 | 6.04 min | 348 |
| thieno[2,3-c] | phenylamine | phenylamino)- | | (a) | (M + H)⁺ |
| pyridine-2- | | thieno | | | |
| carboxylic acid | | [2,3-c] | | | |
| tert-butyl ester | | pyridine-2- | | | |
| (A, B) | | carboxylic acid | | | |
| 4-Bromo- | 4′- | 4-(4′- | 72 | 8.87 min | 415 |
| thieno[2,3-c] | Trifluoromethyl- | Trifluoromethyl- | | (a) | (M + H)⁺ |
| pyridine-2- | biphenyl-4-ylamine | biphenyl-4- | | | |
| carboxylic acid | | ylamino)- | | | |
| tert-butyl ester | | thieno[2,3-c] | | | |
| (A, B) | | pyridine-2- | | | |
| | | carboxylic acid | | | |
| 4-Bromo- | 4′- | 4-(4′- | 73 | 11.20 min | 471 |
| thieno[2,3-c] | Trifluoromethyl- | Trifluoromethyl- | | (b) | (M + H)⁺ |
| pyridine-2- | biphenyl-4- | biphenyl-4- | | | |
| carboxylic acid | ylamine | ylamino)- | | | |
| tert-butyl ester | | thieno[2,3-c] | | | |
| (A, B) | | pyridine-2- | | | |
| | | carboxylic acid | | | |
| | | tert-butyl ester | | | |
| 4-Bromo- | 4-(1H-Pyrazol-4- | 4-[4-(1H-Pyrazol- | 74 | 5.67 min | 337 |
| thieno[2,3-c] | yl)-phenylamine | 4-yl)- | | (a) | (M + H)⁺ |
| pyridine-2- | | phenylamino]- | | | |
| carboxylic acid | | thieno[2,3-c] | | | |
| tert-butyl ester | | pyridine-2- | | | |
| (A, B) | | carboxylic | | | |
| | | acid | | | |
| 4-Bromo- | Biphenyl-4- | 4-(Biphenyl-4- | 75 | 7.99 min | 347 |
| thieno[2,3-c] | ylamine | ylamino)- | | (a) | (M + H)⁺ |
| pyridine-2- | | thieno[2,3-c] | | | |
| carbonitrile (A, | | pyridine-2- | | | |
| C, F) | | carboxylic acid | | | |
| 4-Bromo- | 4-Bromo- | 4-(4-Bromo- | 76 | 7.30 min | 349, 351 |
| thieno[2,3-c] | phenylamine | phenylamino)- | | (a) | (M + H)⁺ |
| pyridine-2- | | thieno[2,3-c] | | | |
| carbonitrile | | pyridine-2- | | | |
| C, F) | | carboxylic acid | | | |
| 4-Bromo- | Phenyl-(2- | 4-[Phenyl-(2- | 77 | 6.04 min | 368 |
| thieno[2,3-c] | pyrrolidin-1-yl- | pyrrolidin-1-yl- | | (a) | (M + H)⁺ |
| pyridine-2- | ethyl)-amine | ethyl)- | | | |
| carboxylic acid | | amino]- | | | |
| tert-butyl ester | | thieno[2,3-c] | | | |
| (A, B) | | pyridine-2- | | | |
| | | carboxylic acid | | | |
| 4-Bromo- | Aniline | 4-Phenylamino- | 78 | 8.07 min | 270.2 |
| thieno{2,3-c] | | thieno[2,3-c] | | (a) | (M + H)⁺ |
| pyridine-2- | | pyridine-2- | | | |
| carboxylic acid | | carboxylic acid | | | |
| amide | | amide | | | |
| (A, C) | | | | | |
| 4-Bromo-2- | 2-Methyl- | (2-Methyl- | 79 | 1.88 min | 385.0 |
| (2H-tetrazol-5- | biphenyl-4- | biphenyl-4-yl)-[2- | | (i) | (M + H)⁺ |
| yl)-thieno[2,3-c] | ylamine | (2H-tetrazol-5- | | | |
| pyridine | (Y) | yl)-thieno[2,3-c] | | | |
| (A,C,F,G) | | pyridin-4-yl]- | | | |
| | | amine | | | |
| 4-Bromo- | 2-Methyl- | 4-(2-Methyl- | 80 | 10.10 min | 360.2 |
| thieno[2,3-c] | biphenyl-4- | biphenyl-4- | | (a) | (M + H)⁺ |
| pyridine-2- | ylamine | ylamino)- | | | |
| carbonitrile | (Y) | thieno[2,3-c] | | | |
| (A,C,F) | | pyridine-2- | | | |
| | | carboxylic acid | | | |
| | | amide | | | |
| 4-Amino- | 4-Imidazol-1-yl | 4-(4-Imidazol-1- | 81 | 1.06 min | 336.18 |
| thieno[2,3-c] | phenylamine | yl-phenylamino)- | | (i) | (M + H)⁺ |
| pyridine-2- | | thieno[2,3-c] | | | |
| carboxylic acid | | pyridine-2- | | | |
| amide (A,C) | | carboxylic acid | | | |
| | | amide acetate salt | | | |
| 4-Amino- | 2-Methyl-5- | 4-(2-Methyl-5- | 82 | 1.68 min | 350.2 |
| thieno[2,3-c] | phenyl-2H- | phenyl-2H- | | (i) | (M + H)⁺ |
| pyridine-2- | pyrazol-3- | pyrazol-3- | | | |
| carboxylic acid | ylamine | ylamino)- | | | |
| amide (A,C) | | thieno[2,3-c] | | | |
| | | pyridine-2- | | | |
| | | carboxylic acid | | | |
| | | amide acetate salt | | | |
| 4-Bromo-2- | tert-Butylaniline | (4-tert-Butyl- | 83 | 1.97 min | 351.1 |
| (1H-tetrazol-5- | | phenyl)-[2-(1H- | | (i) | (M + H)⁺ |
| yl)-thieno[2,3-c] | | tetrazol-5-yl)- | | | |
| pyridine | | thieno[2,3-c] | | | |
| (A,C,F,G) | | pyridin-4-yl]- | | | |
| | | amine | | | |
| 4-Bromo-2- | 5-Phenyl-pyridin- | (5-Phenyl- | 84 | 1.70 min | 372.2 |
| (1H-tetrazol-5- | 2-ylamine | pyridin-2-yl)-[2- | | (i) | (M + H)⁺ |
| yl)-thieno[2,3-c] | | (1H-tetrazol-5- | | | |
| pyridine | | yl)-thieno[2,3-c] | | | |
| (A,C,F,G) | | pyridin-4-yl]- | | | |
| | | amine | | | |
| 4-Bromo- | 4-(1H- | 4-[4-(1H- | 85 | 5.43 min | 386.3 |
| thieno[2,3-c] | Benzoimidazol-2- | Benzoimidazol- | | (j) | (M + H)⁺ |
| pyridine-2- | yl)-phenylamine | 2-yl)-phenylamino]- | | | |
| carboxylic acid | | thieno[2,3-c] | | | |
| amide (A,C) | | pyridine-2- | | | |
| | | carboxylic acid | | | |
| | | amide | | | |
| 4-Bromo- | 4′-Amino- | 4-(4′-Cyano- | 86 | 2.28 min | 371.2 |
| thieno[2,3-c] | biphenyl-4- | biphenyl-4- | | (i) | (M + H)⁺ |
| pyridine-2- | carbonitrile | ylamino)- | | | |
| carboxylic acid | | thieno[2,3-c] | | | |
| amide (A,C) | | pyridine-2- | | | |
| | | carboxylic acid | | | |
| | | amide | | | |
| 4-Bromo- | 4-Morpholin-4- | N-(2- | 87 | 1.55 min | 531.2 |
| thieno[2,3-c] | yl-phenylamine | Carboxamido- | | (i) | (M + H)⁺ |
| pyridine-2- | | thieno[2,3-c] | | | |
| carboxylic acid | | pyrid-4-yl)-2-[(4- | | | |
| amide (A,C) | | morpholin-4-yl- | | | |
| | | phenylamino)- | | | |
| | | thieno[2,3-c] | | | |
| | | pyridine]amide | | | |
| 4-Bromo- | 4-Morpholin-4- | 4-(4-Morpholin- | 88 | 1.25 min | 355.2 |
| thieno[2,3-c] | yl-phenylamine | 4-yl- | | (i) | (M + H)⁺ |
| pyridine-2- | | phenylamino)- | | | |
| carboxylic acid | | thieno[2,3-c] | | | |
| amide (A,C) | | pyridine-2- | | | |
| | | carboxylic acid | | | |
| | | amide | | | |
| 4-Bromo- | 4-Cyclohexyl- | N-(2- | 89 | 2.96 min | 528.2 |
| thieno[2,3-c] | phenylamine | Carboxamido- | | (i) | (M + H)⁺ |
| pyridine-2- | | thieno[2,3-c] | | | |
| carboxylic acid | | pyrid-4-yl)-2-[(4- | | | |
| amide (A,C) | | cyclohexyl- | | | |
| | | phenylamino)- | | | |
| | | thieno[2,3-c] | | | |
| | | pyridine]amide | | | |
| 4-Bromo- | 4-Cyclohexyl- | 4-(4-Cyclohexyl- | 90 | 2.93 min | 352.18 |
| thieno[2,3-c] | phenylamine | phenylamino)- | | (i) | (M + H)⁺ |
| pyridine-2- | | thieno[2,3-c] | | | |
| carboxylic acid | | pyridine-2- | | | |
| amide (A,C) | | carboxylic acid | | | |
| | | amide | | | |
| 4-Bromo- | 5-Phenyl-pyridin- | 4-(5-Phenyl- | 91 | 8.9 min | 347.1 |
| thieno[2,3-c] | 2-ylamine | pyridin-2- | | (a) | (M + H)⁺ |
| pyridine-2- | | ylamino)- | | | |
| carboxylic acid | | thieno[2,3-c] | | | |
| amide (A,C) | | pyridine-2- | | | |
| | | carboxylic acid | | | |
| | | amide | | | |
| 4-Bromo- | tert-Butylaniline | 4-(4-tert-Butyl- | 92 | 10.05 min | 326.4 |
| thieno[2,3-c] | | phenylamino)- | | (a) | (M + H)⁺ |
| pyridine-2- | | thieno[2,3-c] | | | |
| carboxylic acid | | pyridine-2- | | | |
| amide (A,C) | | carboxylic acid | | | |
| | | amide | | | |
| 4-Bromo- | 4-Phenylaniline | 4-(Biphenyl-4- | 93 | 5.49 min | 443.9 |
| pyrrolo[2,3-c] | | ylamino)- | | (n) | (M + H)⁺ |
| pyridine-1,2- | | pyrrolo[2,3-c] | | | |
| dicarboxylic | | pyridine-1,2- | | | |
| acid 1-tert-butyl | | dicarboxylic acid | | | |
| ester 2-methyl | | 1-tert-butyl ester | | | |
| ester (Z, AA) | | 2-methyl ester | | | |
| 8-Bromo- | Biphenyl-4- | 8-(Biphenyl-4- | 94 | 2.30 min | 342.1 |
| [1,6]naphthyr- | ylamine | ylamino)- | | (m) | (M + H)⁺ |
| idine-2- | | [1,6]naphthyridine- | | | |
| carboxylic acid | | 2-carboxylic | | | |
| | | acid | | | |
| 4-Bromo- | 4-tert-Butyl- | 4-(4-tert-Butyl- | 95 | 11.25 min | 381.2 |
| thieno[2,3-c] | phenylamine | phenylamino)- | | (b) | (M − H)⁻ |
| pyridine-2- | | thieno[2,3-c] | | | |
| carboxylic acid | | pyridine-2- | | | |
| tert-butyl ester | | carboxylic acid | | | |
| (A, B) | | tert-butyl ester | | | |
| 4-(3-Amino- | Bromobenzene | 4-(3- | 96 | 9.87 min | 346 |
| phenyl)- | | Phenylamino- | | (a) | (M + H)⁺ |
| thieno[2,3-c] | | phenyl)- | | | |
| pyridine-2- | | thieno[2,3-c] | | | |
| carboxylic acid | | pyridine-2- | | | |
| amide | | carboxylic acid | | | |
| (A,C) | | amide | | | |
| 4-Bromo- | 4-Bromoaniline | 4-(4-Bromo- | 97 | 1.75 min | 348.2 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 4-Bromoaniline | 4-(4-Bromo- | 98 | 2.27 min | 347.2 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| (C, F) | | carboxylic acid | | | |
| | | amide | | | |
| 4-Bromo- | 4-Nitroaniline | 4-(4-Nitro- | 99 | 2.04 min | 313.3 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 3,4- | 4-(3,4-Dichloro- | 100 | 2.45 min | 337.2 |
| thieno[2,3-c] | Dichloroaniline | phenylamino)- | | (l) | (M − H)⁻ |
| pyridine-2- | | thieno]2,3-c] | | | |
| carbonitrile (A, | | pyridine-2- | | | |
| C, F) | | carboxylic acid | | | |
| | | amide | | | |
| 4-Bromo- | 5-Amino-2- | 4-(4-Nitro-3- | 101 | 2.33 min | 381.3 |
| thieno[2,3-c] | nitrobenzotri- | trifluoromethyl- | | (l) | (M − H)⁻ |
| pyridine-2- | fluoride | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 3,4- | 4-(2,3-Dihydro- | 102 | 1.94 min | 326.4 |
| thieno[2,3-c] | Ethylenedioxy- | benzo[1,4]dioxin- | | (l) | (M − H)⁻ |
| pyridine-2- | aniline | 6-ylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4- | 4-(4- | 103 | 2.34 min | 336.3 |
| thieno[2,3-c] | Aminobenzotri- | Trifluoromethyl- | | (l) | (M − H)⁻ |
| pyridine-2- | fluoride | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Fluoroaniline | 4-(4-Fluoro- | 104 | 2.03 min | 286.3 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Chloroaniline | 4-(4-Chloro- | 105 | 2.22 min | 302.8 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | p-Phenetidine | 4-(4-Ethoxy- | 106 | 2.14 min | 312.4 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | p-Toluidine | 4-p-Tolylamino- | 107 | 2.15 min | 282.4 |
| thieno[2,3-c] | | thieno[2,3-c] | | (l) | (M − H)⁻ |
| pyridine-2- | | pyridine-2- | | | |
| carbonitrile (A, | | carboxylic acid | | | |
| C, F) | | amide | | | |
| 4-Bromo- | 5-Aminoindan | 4-(Indan-5- | 108 | 2.36 min | 308.4 |
| thieno[2,3-c] | | ylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Ethylaniline | 4-(4-Ethyl- | 109 | 2.24 min | 296.4 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Bromo-2- | 4-(4-Bromo-2- | 110 | 2.33 min | 365.2 |
| thieno[2,3-c] | fluoroaniline | fluoro- | | (l) | (M − H)⁻ |
| pyridine-2- | | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 3,4- | 4-(3,4-Dichloro- | 111 | 1.87 min | 338.2 |
| thieno[2,3-c] | Dichloroaniline | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 5-Amino-2- | 4-(4-Nitro-3- | 112 | 1.77 min | 382.3 |
| thieno[2,3-c] | nitrobenzotri- | trifluoromethyl- | | (l) | (M − H)⁻ |
| pyridine-2- | fluoride | phenylamino)- | | | |
| carbonitrile (A, | | thieno[2,3-c] | | | |
| C, F) | | pyridine-2- | | | |
| | | carboxylic acid | | | |
| 4-Bromo- | 4- | 4-(4- | 113 | 1.88 min | 337.3 |
| thieno[2,3-c] | Aminobenzotri- | Trifluoromethyl- | | (l) | (M − H)⁻ |
| pyridine-2- | fluoride | phenylamino)- | | | |
| carbonitrile (A, | | thieno[2,3-c] | | | |
| C, F) | | pyridine-2- | | | |
| | | carboxylic acid | | | |
| 4-Bromo- | 4- | 4-(4-Cyano- | 114 | 1.46 min | 294.3 |
| thieno[2,3-c] | Aminobenzoni- | phenylamino)- | | (l) | (M − H)⁻ |
| pyridine-2- | trile | thieno[2,3-c] | | | |
| carbonitrile (A, | | pyridine-2- | | | |
| C, F) | | carboxylic acid | | | |
| 4-Bromo- | 4-Fluoroaniline | 4-(4-Fluoro- | 115 | 1.51 min | 287.3 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 4-Chloroaniline | 4-(4-Chloro- | 116 | 1.69 min | 303.8 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | N,N-Dimethyl-p- | 4-(4- | 117 | 1.43 min | 312.4 |
| thieno[2,3-c] | phenylenedi- | Dimethylamino- | | (l) | (M − H)⁻ |
| pyridine-2- | amine | phenylamino)- | | | |
| carbonitrile (A, | | thieno[2,3-c] | | | |
| C, F) | | pyridine-2- | | | |
| | | carboxylic acid | | | |
| 4-Bromo- | p-Anisidine | 4-(4-Methoxy- | 118 | 1.45 | 299.3 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | p-Phenetidine | 4-(4-Ethoxy- | 119 | 1.69 min | 313.4 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | p-Toluidine | 4-p-Tolylamino- | 120 | 1.61 min | 283.3 |
| thieno[2,3-c] | | thieno[2,3-c] | | (l) | (M − H)⁻ |
| pyridine-2- | | pyridine-2- | | | |
| carbonitrile (A, | | carboxylic acid | | | |
| C, F) | | | | | |
| 4-Bromo- | 5-Aminoindan | 4-(Indan-5- | 121 | 1.85 min | 309.4 |
| thieno[2,3-c] | | ylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 4-Ethylaniline | 4-(4-Ethyl- | 122 | 1.7 min | 297.4 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 4-Bromo-2- | 4-(4-Bromo-2- | 123 | 1.74 min | 366.2 |
| thieno[2,3-c] | fluoroaniline | fluoro- | | (l) | (M − H)⁻ |
| pyridine-2- | | phenylamino)- | | | |
| carbonitrile (A, | | thieno[2,3-c] | | | |
| C, F) | | pyridine-2- | | | |
| | | carboxylic acid | | | |
| 4-Bromo- | 5-Amino-2- | 4-(2-Methyl-1H- | 124 | 1.57 min | 322.4 |
| thieno[2,3-c] | methylindole | indol-5-ylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 5-Aminoindole | 4-(1H-Indol-5- | 125 | 1.42 min | 308.3 |
| thieno[2,3-c] | | ylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| 4-Bromo- | 3,4- | 4- | 126 | 1.44 min | 313.3 |
| thieno[2,3-c] | (Methylenedioxy) | (Benzo[1,3]dioxol- | | (l) | (M − H)⁻ |
| pyridine-2- | aniline | 5-ylamino)- | | | |
| carbonitrile (A, | | thieno[2,3-c] | | | |
| C, F) | | pyridine-2- | | | |
| | | carboxylic acid | | | |
| 4-Bromo- | 4- | 4-(4-Morpholin- | 127 | 1.42 min | 354.4 |
| thieno[2,3-c] | Morpholino- | 4-yl- | | (l) | (M − H)⁻ |
| pyridine-2- | aniline | phenylamino)- | | | |
| carbonitrile (A, | | thieno[2,3-c] | | | |
| C, F) | | pyridine-2- | | | |
| | | carboxylic acid | | | |
| 4-Bromo- | 4′- | 4-(4-Acetyl- | 128 | 1.41 min | 311.3 |
| thieno[2,3-c] | Aminoaceto- | phenylamino)- | | (l) | (M − H)⁻ |
| pyridine-2- | phenone | thieno[2,3-c] | | | |
| carbonitrile (A, | | pyridine-2- | | | |
| C, F) | | carboxylic acid | | | |
| 4-Bromo- | 4- | 4-(4- | 129 | 2.44 min | 352.3 |
| thieno[2,3-c] | (Trifluoromethoxy) | Trifluoromethoxy- | | (l) | (M − H)⁻ |
| pyridine-2- | aniline | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 5-Amino-2- | 4-(2-Methyl- | 130 | 2.00 min | 339.4 |
| thieno[2,3-c] | methylbenzo- | benzothiazole-5- | | (l) | (M − H)⁻ |
| pyridine-2- | thiazole | ylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Amino-2- | 4-(3- | 131 | 1.74 min | 369.5 |
| thieno[2,3-c] | diethylamino- | Diethylaminomethyl- | | (l) | (M − H)⁻ |
| pyridine-2- | methyl-phenol | 4-hydroxy- | | | |
| carbonitrile (A, | | phenylamino)- | | | |
| C, F) | | thieno[2,3-c] | | | |
| | | pyridine-2- | | | |
| | | carboxylic acid | | | |
| | | amide | | | |
| 4-Bromo- | 4- | 4-(4- | 132 | 1.66 min | 339.4 |
| thieno[2,3-c] | (Dimethylcarbamyl) | Dimethylcarbamoyl- | | (l) | (M − H)⁻ |
| pyridine-2- | aniline | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Bromo-3- | 4-(4-Bromo-3- | 133 | 2.36 min | 365.2 |
| thieno[2,3-c] | fluoroaniline | fluoro- | | (l) | (M − H)⁻ |
| pyridine-2- | | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 1-Acetyl-6- | 4-(1-Acetyl-2,3- | 134 | 1.85 min | 351.4 |
| thieno[2,3-c] | amino-2,3- | dihydro-1H- | | (l) | (M − H)⁻ |
| pyridine-2- | dihydro-(1h)- | indol-6-ylamino)- | | | |
| carbonitrile (A, | indole | thieno[2,3-c] | | | |
| C, F) | | pyridine-2- | | | |
| | | carboxylic acid | | | |
| | | amide | | | |
| 4-Bromo- | 4-Aminostyrene | 4-(4-Vinyl | 135 | 2.28 min | 294.4 |
| thieno[2,3-c] | | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Aminobenzyl | 4-(4- | 136 | 1.88 min | 307.4 |
| thieno[2,3-c] | cyanide | Cyanomethyl- | | (l) | (M − H)⁻ |
| pyridine-2- | | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Propoxy- | 4-(4-Propoxy- | 137 | 2.39 min | 326.4 |
| thieno[2,3-c] | phenylamine | phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4- | 4-(4- | 138 | 2.11 min | 387.5 |
| thieno[2,3-c] | Aminobenzanilide | Phenylcarbamoyl- | | (l) | (M − H)⁻ |
| pyridine-2- | | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4- | 4-(4- | 139 | 2.2 min | 334.3 |
| thieno[2,3-c] | (Difluoromethoxy) | Difluoromethoxy- | | (l) | (M − H)⁻ |
| pyridine-2- | aniline | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Amino-benzoic | 4-(2-Carbamoyl- | 140 | 2.16 min | 340.4 |
| thieno[2,3-c] | acid ethyl ester | thieno[2,3-c] | | (l) | (M − H)⁻ |
| pyridine-2- | | pyridin-4- | | | |
| carbonitrile (A, | | ylamino)-benzoic | | | |
| C, F) | | acid ethyl ester | | | |
| 4-Bromo- | 4-Piperidin-1-yl- | 4-(4-Piperidin-1- | 141 | 2.03 min | 351.5 |
| thieno[2,3-c] | phenylamine | yl-phenylamino)- | | (l) | (M − H)⁻ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Chloro-3- | 4-(4-Chloro-3- | 142 | 2.5 min | 370.8 |
| thieno[2,3-c] | trifluoromethyl- | trifluoromethyl- | | (l) | (M − H)⁻ |
| pyridine-2- | phenylamine | phenylamino)- | | | |
| carbonitrile (A, | | thieno[2,3-c] | | | |
| C, F) | | pyridine-2- | | | |
| | | carboxylic acid | | | |
| | | amide | | | |
| 4-Bromo- | (4-Amino- | 4-[4-(Cyano- | 143 | 2.26 min | 383.5 |
| thieno[2,3-c] | phenyl)-phenyl- | phenyl-methyl)- | | (l) | (M − H)⁻ |
| pyridine-2- | acetonitrile | phenylamino]- | | | |
| carbonitrile (A, | | thieno[2,3-c] | | | |
| C, F) | | pyridine-2- | | | |
| | | carboxylic acid | | | |
| | | amide | | | |
| 4-Bromo- | 4-Bromoaniline | 4-(4-Bromo- | 144 | 1.75 min | 348.2 |
| thieno[2,3-c] | | phenylamino)- | | (a) | (M − H)⁻ |
| pyridine-2- | | thieno[2,3-c] | | | |
| carbonitrile (A, | | pyridine-2- | | | |
| C, F) | | carboxylic acid | | | |
| 4-(4-Bromo- | 3′-Cyano- | 4-(3′-Cyano- | 145 | 9.25 min | 369.4 |
| phenylamino)- | biphenyl-4- | biphenyl-4- | | (a) | (M − H)⁻ |
| thieno[2,3-c] | ylamino | ylamino)- | | | |
| pyridine-2- | | thieno[2,3-c] | | | |
| carboxylic acid | | pyridine-2- | | | |
| amide (I (A, C, | | carboxylic acid | | | |
| F) | | amide | | | |
| 4-Bromo- | Cyclohexylamine | 4- | 145 | 8.39 | 276.2 |
| thieno[2,3-c] | | Cyclohexylamino- | A | (a) | (M + H)+ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | 4-Phenyl- | 4-(4-Phenyl- | 145 | 10.00 (a) | 352.2 |
| thieno[2,3-c] | cyclohexylamine | cyclohexylamino)- | B | 10.19 (a) | (M + H)+ |
| pyridine-2- | | thieno[2,3-c] | | | |
| carbonitrile (A, | | pyridine-2- | | | |
| C, F) | | carboxylic acid | | | |
| | | amide | | | |
| 4-Bromo- | (1R,5R)-3,6- | (1R,5R)-6-(2- | 145 | 8.27 (a) | 409.2 |
| thieno[2,3-c] | Diaza- | Carbamoyl- | C | | (M + H)+ |
| pyridine-2- | bicyclo[3.2.0] | thieno[2,3-c] | | | |
| carbonitrile (A, | heptane-3-carboxylic | pyridin-4-yl)- | | | |
| C, F) | acid benzyl ester | 3,6-diaza- | | | |
| | | bicyclo[3.2.0] | | | |
| | | heptane-3- | | | |
| | | carboxylic acid | | | |
| | | benzyl ester | | | |
| 4-Bromo- | (R)-3-Amino- | (R)-3-(2- | 145 | 8.19 (a) | 363.0 |
| thieno[2,3-c] | pyrrolidine-1- | Carbamoyl- | D | | (M + H)+ |
| pyridine-2- | carboxylic acid | thieno[2,3-c] | | | |
| carbonitrile (A, | tert-butyl ester | pyridine-4-ylamino)- | | | |
| C, F) | | pyrrolidine-1- | | | |
| | | carboxylic acid | | | |
| | | tert-butyl ester | | | |
| 4-Bromo- | 2-Benzyl- | 4-(5-Benzyl- | 145 | 6.70 (a) | 379.2 |
| thieno[2,3-c] | octahydro- | hexahydro- | E | | (M + H)+ |
| pyridine-2- | pyrrolo[3,4-c] | pyrrolo[3,4-c] | | | |
| carbonitrile (A, | pyrrole | pyrrol-2-yl)- | | | |
| C, F) | | thieno]2,3-c] | | | |
| | | pyridine-2- | | | |
| | | carboxylic acid | | | |
| | | amide | | | |
| 4-Bromo- | (3aR,7aR)-2- | 4-((3aS,7aR)-2- | 145 | 6.95 (a) | 393.2 |
| thieno[2,3-c] | Benzyl- | Benzyl- | F | | (M + H) |
| pyridine-2- | octahydro-pyrrolo | octahydro-pyrrolo | | | |
| carbonitrile (A, | [3,4-c]pyridine | [3,4-c]pyridin- | | | |
| C, F) | | 5-yl)-thieno[2,3-c] | | | |
| | | pyridine-2- | | | |
| | | carboxylic acid | | | |
| | | amide | | | |
| 4-Bromo- | 2-Benzyl-2,8- | 4-(2-Benzyl-1- | 145 | 8.72 (a) | 421.3 |
| thieno[2,3-c] | diaza- | oxo-2,8-diaza- | G | | (M + H)+ |
| pyridine-2- | spiro[4.5]decan- | spiro[4.5] | | | |
| carbonitrile (A, | 1-one | dec-8-yl)- | | | |
| C, F) | | thieno[2,3-c] | | | |
| | | pyridine-2- | | | |
| | | carboxylic acid | | | |
| | | amide | | | |
| 4-Bromo- | 2-Phenyl-2,8- | 4-(1-Oxo-2- | 145 | 8.85 (a) | 407.3 |
| thieno[2,3-c] | diaza- | phenyl-2,8-diaza- | H | | (M + H)+ |
| pyridine-2- | spiro[4.5]decan- | spiro[4.5] | | | |
| carbonitrile (A, | 1-one | dec-8-yl)- | | | |
| C, F) | | thieno[2,3-c] | | | |
| | | pyridine-2- | | | |
| | | carboxylic acid | | | |
| | | amide | | | |
| 4-Bromo- | (1R,4S)-2,5- | (1R,4S)-5-(2- | 145 | 8.12 (a) | 375.1 |
| thieno[2,3-c] | Diaza- | Carbamoyl- | I | | (M + H)+ |
| pyridine-2- | bicyclo[2.2.1] | thieno[2,3-c] | | | |
| carbonitrile (A, | heptane-2- | pyridin-4-yl)- | | | |
| C, F) | carboxylic acid | 2,5-diaza-bicyclo | | | |
| | tert-butyl ester | [2.2.1]heptane-2- | | | |
| | | carboxylic acid | | | |
| | | tert-butyl ester | | | |
| 4-Bromo- | 1-Benzyl- | 4-(1-Benzyl- | 145 | 6.65 (a) | 367.2 |
| thieno[2,3-c] | piperidin-4- | piperidin-4- | J | | (M + H)+ |
| pyridine-2- | ylamine | ylamino)- | | | |
| carbonitrile | | thieno[2,3-c] | | | |
| | | pyridine-2- | | | |
| | | carboxylic acid | | | |
| | | amide | | | |
| 4-Bromo- | 1-Benzyl- | 4-(1-Benzyl- | 145 | 5.55 (a) | 368.2 |
| thieno[2,3-c] | piperidin-4- | piperidin-4- | K | | (M + H)+ |
| pyridine-2- | ylamine | ylamino)- | | | |
| carbontrile | | thieno[2,3-c] | | | |
| | | pyridine-2- | | | |
| | | carboxylic acid | | | |
| 4-Bromo- | 8-Benzyl-8-aza- | 4-(8-Benzyl-8- | 145 | 4.24 (a) | 393.2 |
| thieno[2,3-c] | bicyclo[3.2.1] | aza- | L | | (M + H)⁺ |
| pyridine-2- | oct-3-ylamine | bicyclo[3.2.1] | | | |
| carbontrile | | oct-3-ylamino)- | | | |
| | | thieno[2,3-c] | | | |
| | | pyridine-2- | | | |
| | | carboxylic acid | | | |
| | | amide | | | |
| 4-Bromo- | 4-Amino- | 4-(2-Carbamoyl- | 145 | 8.46 (a) | 359.0 |
| thieno[2,3-c] | piperidine-1- | thieno[2,3-c] | M | | (M + H)+ |
| pyridine-2- | carboxylic acid | pyridin-4- | | | |
| carbonitrile | tert-butyl ester | ylamino)- | | | |
| | piperidine-1- | | | | |
| | | carboxylic acid | | | |
| | | tert-butyl ester | | | |
| 4-Bromo- | 4-Amino- | 4-(2-Carbamoyl- | 145 | 8.46 (a) | 359.0 |
| thieno[2,3-c] | piperdine-1- | thieno[2,3-c] | N | | (M + H)+ |
| pyridine-2- | carboxylic acid | pyridin-4- | | | |
| carbonitrile | tert-butyl ester | ylamino)- | | | |
| | | piperidine-1- | | | |
| | | carboxylic acid | | | |
| | | tert-butyl ester | | | |
| 4-Bromo- | 1-Phenyl- | 4-(1-Phenyl- | 145 | 5.38 (a) | 353 |
| thieno[2,3-c] | piperdin-4- | piperdin-4- | O | | (M + H) |
| pyridine-2- | ylamine | ylamino)- | | | |
| carbontrile | | thieno[2,3-c] | | | |
| | | pyridine-2- | | | |
| | | carboxylic acid | | | |
| | | amide | | | |

Other compounds obtained using general procedure J are shown in Table 8.

TABLE 8
Examples synthesized using General Procedure J

| Iodide or | | | | | m/z or |
|---|---|---|---|---|---|
| Bromide | Boronate | | | HPLC Rₜ | ¹H |
| Precursor | Precursor | Product | Ex # | (Method) | NMR δ |
| 4-Bromo-2- | 1- | 4-Naphthalen-1- | 146 | 2.64 min | 329 |
| (1H-tetrazol- | Naphthalene- | yl-2-(1H-tetrazol- | | (m) | (M + H)⁺ |
| 5-yl)-thieno | boronic acid | 5-yl)-thieno[2,3-c] | | | |
| [2,3-c]pyridine | | pyridine | | | |
| (A, C, F, G) | | | | | |
| 4-Bromo-2- | 5-(4,4,5,5- | 4- | 147 | 2.95 min | 367 |
| (1H-tetrazol- | Tetramethyl- | [2,2′]Bithiophenyl- | | (m) | (M + H)⁺ |
| 5-yl)-thieno | 1,3,2- | 5-yl-2-(1H- | | | |
| [2,3-c] | dioxaborolan- | tetrazol-5-yl)- | | | |
| pyridine | 2-yl)-2,2′- | thieno[2,3-c] | | | |
| (A, C, F, G) | bithiophene | pyridine | | | |
| 4-Bromo- | 4- | 4-(4- | 148 | 2.47 min | 321.3 |
| thieno[2,3-c] | (Trifluoromethyl) | Trifluoromethyl- | | (m) | (M + H)⁻ |
| pyridine-2- | phenylboronic | phenyl)-thieno | | | |
| carboxylic | acid | [2,3-c]pyridine-2- | | | |
| acid amide | | carboxylic acid | | | |
| (A, C) | | amide | | | |
| 4-Bromo- | 4- | 4-(4-Chloro- | 149 | 2.34 min | 287.8 |
| thieno[2,3-c] | Chlorophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-(3- | 150 | 2.42 min | 321.3 |
| thieno[2,3-c] | (Trifluoromethyl) | Trifluoromethyl- | | (m) | (M + H)⁻ |
| pyridine-2- | phenylboronic | phenyl)-thieno | | | |
| carboxylic | acid | [2,3-c]pyridine-2- | | | |
| acid amide | | carboxylic acid | | | |
| (A, C) | | amide | | | |
| 4-Bromo- | 3,5- | 4-(3,5-Dichloro- | 151 | 2.58 min | 322.2 |
| thieno[2,3-c] | Dichlorophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Amino- | 152 | 1.72 min | 268.3 |
| thieno[2,3-c] | Aminophenyl- | phenyl)- | | (m) | (M + H)⁻ |
| pyridine-2- | boronic acid | thieno[2,3-c] | | | |
| carboxylic | hemisulfate | pyridine-2- | | | |
| acid amide | | carboxylic acid | | | |
| (A, C) | | amide | | | |
| 4-Bromo- | 4- | 4-(4-Amino- | 153 | 1.67 min | 268.3 |
| thieno[2,3-c] | Aminophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | Pyridine-4- | 4-Pyridin-4-yl- | 154 | 1.52 min | 254.3 |
| thieno[2,3-c] | boronic acid | thieno[2,3-c] | | (m) | (M + H)⁻ |
| pyridine-2- | | pyridine-2- | | | |
| carboxylic | | carboxylic acid | | | |
| acid amide | | amide | | | |
| (A, C) | | | | | |
| 4-Bromo- | o- | 4-o-Tolyl- | 155 | 2.19 min | 267.3 |
| thieno[2,3-c] | Tolyboronic | thieno[2,3-c] | | (m) | (M + H)⁻ |
| pyridine-2- | acid | pyridine-2- | | | |
| carboxylic | | carboxylic acid | | | |
| acid amide | | amide | | | |
| (A, C) | | | | | |
| 4-Bromo- | 4- | 4-p-Tolyl- | 156 | 2.27 min | 267.3 |
| thieno[2,3-c] | Methylphenyl- | thieno[2,3-c] | | (m) | (M + H)⁻ |
| pyridine-2- | boronic acid | pyridine-2- | | | |
| carboxylic | | carboxylic acid | | | |
| acid amide | | amide | | | |
| (A, C) | | | | | |
| 4-Bromo- | 4- | 4-(4-Hydroxy- | 157 | 1.72 min | 269.3 |
| thieno[2,3-c] | Hydroxyphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Hydroxy- | 158 | 1.77 min | 269.3 |
| thieno[2,3-c] | Hydroxyphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 2,5- | 4-(2,5-Dimethyl- | 159 | 2.39 min | 281.4 |
| thieno[2,3-c] | Dimethylphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 4- | 4-(4- | 160 | 1.66 min | 283.3 |
| thieno[2,3-c] | (Hydroxymethyl) | Hydroxymethyl- | | (m) | (M + H)⁻ |
| pyridine-2- | phenylboronic | phenyl)-thieno[2,3-c] | | | |
| carboxylic | acid | pyridine-2- | | | |
| acid amide | | carboxylic acid | | | |
| (A, C) | | amide | | | |
| 4-Bromo- | (3- | 4-(3- | 161 | 1.69 min | 283.3 |
| thieno[2,3-c] | Hydroxymethyl- | Hydroxymethyl- | | (m) | (M + H)⁻ |
| pyridine-2- | phenyl) | phenyl)- | | | |
| carboxylic | boronic acid | thieno[2,3-c] | | | |
| acid amide | | pyridine-2- | | | |
| (A, C) | | carboxylic acid | | | |
| | | amide | | | |
| 4-Bromo- | 4- | 4-(4-Methoxy- | 162 | 2.10 min | 283.3 |
| thieno[2,3-c] | Methoxyphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | (2- | 4-(2- | 163 | 1.67 min | 283.3 |
| thieno[2,3-c] | Hydroxymethyl- | Hydroxymethyl- | | (m) | (M + H)⁻ |
| pyridine-2- | phenyl)boronic | phenyl)-thieno[2,3-c] | | | |
| carboxylic | acid dehydrate | pyridine-2- | | | |
| acid amide | | carboxylic acid | | | |
| (A, C) | | amide | | | |
| 4-Bromo- | 5-Fluoro-2- | 4-(5-Fluoro-2- | 164 | 2.15 min | 301.3 |
| thieno[2,3-c] | methoxyphenyl- | methoxy-phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 2-Fluoro-3- | 4-(2-Fluoro-3- | 165 | 2.09 min | 301.3 |
| thieno[2,3-c] | methoxyphenyl- | methoxy-phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 1- | 4-Naphthalen-1- | 166 | 2.41 min | 303.4 |
| thieno[2,3-c] | Naphthalene- | yl-thieno[2,3-c] | | (m) | (M + H)⁻ |
| pyridine-2- | boronic acid | pyridine-2- | | | |
| carboxylic | | carboxylic acid | | | |
| acid amide | | amide | | | |
| (A, C) | | | | | |
| 4-Bromo- | Benzo[b]thiophene- | 4-Benzo[b]thiopen- | 167 | 2.54 min | 309.4 |
| thieno[2,3-c] | 2-boronic acid | 2-yl-thieno[2,3-c] | | (m) | (M + H)⁻ |
| pyridine-2- | | pyridine-2- | | | |
| carboxylic | | carboxylic acid | | | |
| acid amide | | amide | | | |
| (A, C) | | | | | |
| 4-Bromo- | 3,4- | 4-(3,4- | 168 | 1.95 min | 313.4 |
| thieno[2,3-c] | Dimethoxyphenyl- | Dimethoxy- | | (m) | (M + H)⁻ |
| pyridine-2- | boronic acid | phenyl)- | | | |
| carboxylic | | thieno[2,3-c] | | | |
| acid amide | | pyridine-2- | | | |
| (A, C) | | carboxylic acid | | | |
| | | amide | | | |
| 4-Bromo- | 3,4-Dihydro- | 4-(3,4-Dihydro- | 169 | 2.14 min | 325.4 |
| thieno[2,3-c] | 2h-1,5- | 2H- | | (m) | (M + H)⁻ |
| pyridine-2- | benzodioxepin- | benzo[b][1,4] | | | |
| carboxylic | 7-ylboronic acid | dioxepin-7-yl)- | | | |
| acid amide | | thieno[2,3-c] | | | |
| (A, C) | | pyridine-2- | | | |
| | | carboxylic acid | | | |
| 4-Bromo- | 2- | 4-Biphenyl-2-yl- | 170 | 2.49 min | 329.4 |
| thieno[2,3-c] | Biphenylboronic | thieno[2,3-c] | | (m) | (M + H)⁻ |
| pyridine-2- | acid | pyridine-2- | | | |
| carboxylic | | carboxylic acid | | | |
| acid amide | | amide | | | |
| (A, C) | | | | | |
| 4-Bromo- | 2- | 4-(2-Chloro- | 171 | 2.18 min | 287.8 |
| thieno[2,3-c] | Chlorophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 2,3- | 4-(2,3-Difluoro- | 172 | 2.15 min | 289.3 |
| thieno[2,3-c] | Difluorophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | Benzo[b]furan-2- | 4-Benzofuran-2- | 173 | 2.41 min | 293.3 |
| thieno[2,3-c] | boronic acid | yl-thieno | | (m) | (M + H)⁻ |
| pyridine-2- | | [2,3-c] | | | |
| carboxylic | | pyridine-2- | | | |
| acid amide | | carboxylic acid | | | |
| (A, C) | | amide | | | |
| 4-Bromo- | 4- | 4-(4-Acetyl- | 174 | 1.95 min | 295.4 |
| thieno[2,3-c] | Acetylphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 2,3-Dihydrdo- | 4-(2,3-Dihydro- | 175 | 2.09 min | 295.4 |
| thieno[2,3-c] | 1-benzofuran- | benzofuran-5-yl)- | | (m) | (M + H)⁻ |
| pyridine-2- | 5-ylboronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Isopropyl- | 176 | 2.61 min | 295.4 |
| thieno[2,3-c] | Isopropylphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 4-(N,N- | 4-(4- | 177 | 2.26 min | 296.4 |
| thieno[2,3-c] | Dimethylamino) | Dimethylamino- | | (m) | (M + H)⁻ |
| pyridine-2- | phenylboronic | phenyl)-thieno[2,3-c] | | | |
| carboxylic | acid | pyridine-2- | | | |
| acid amide | | carboxylic acid | | | |
| (A, C) | | amide | | | |
| 4-Bromo- | 3,4- | 4- | 178 | 2.08 min | 297.3 |
| thieno[2,3-c] | Methylenedioxy- | Benzo[1,3]dioxol- | | (m) | (M + H)⁻ |
| pyridine-2- | phenylboronic acid | 5-yl-thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Ethoxy- | 179 | 2.31 min | 297.4 |
| thieno[2,3-c] | Ethoxyphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Nitro- | 180 | 2.10 min | 298.3 |
| thieno[2,3-c] | Nitrophenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 4- | 4-(4- | 181 | 1.76 min | 331.4 |
| thieno[2,3-c] | (Methanesulfonyl) | Methanesulfonyl- | | (m) | (M + H)⁻ |
| pyridine-2- | phenyl- | phenyl)-thieno[2,3-c] | | | |
| carboxylic | boronic acid | pyridine-2- | | | |
| acid amide | | carboxylic acid | | | |
| (A, C) | | amide | | | |
| 4-Bromo- | 3- | 4-(3- | 182 | 2.50 min | 337.3 |
| thieno[2,3-c] | (Trifluoromethoxy) | Trifluoromethoxy- | | (m) | (M + H)⁻ |
| pyridine-2- | benzeneboronic | phenyl)- | | | |
| carboxylic | acid | thieno[2,3-c] | | | |
| acid amide | | pyridine-2- | | | |
| (A, C) | | carboxylic acid | | | |
| | | amide | | | |
| 4-Bromo- | 4- | 4-(4- | 183 | 2.53 min | 337.3 |
| thieno[2,3-c] | (Trifluoromethoxy) | Trifluoromethoxy- | | (m) | (M + H)⁻ |
| pyridine-2- | benzeneboronic | phenyl)- | | | |
| carboxylic | acid | thieno[2,3-c] | | | |
| acid amide | | pyridine-2- | | | |
| (A, C) | | carboxylic acid | | | |
| | | amide | | | |
| 4-Bromo- | 2- | 4-(2- | 184 | 2.75 min | 337.3 |
| thieno[2,3-c] | (Trifluoromethoxy) | Trifluoromethoxy- | | (m) | (M + H)⁻ |
| pyridine-2- | benzeneboronic | phenyl)- | | | |
| carboxylic | acid | thieno[2,3-c] | | | |
| acid amide | | pyridine-2- | | | |
| (A, C) | | carboxylic acid | | | |
| | | amide | | | |
| 4-Bromo- | (2- | 4-(2-Phenoxy- | 185 | 2.51 min | 345.4 |
| thieno[2,3-c] | Phenoxy) | phenyl)- | | (m) | (M + H)⁻ |
| pyridine-2- | phenylboronic | thieno[2,3-c] | | | |
| carboxylic | acid | pyridine-2- | | | |
| acid amide | | carboxylic acid | | | |
| (A, C) | | amide | | | |
| 4-Bromo- | 2- | 4-(2-Fluoro- | 186 | 2.34 min | 347.4 |
| thieno[2,3-c] | Fluorobiphenyl- | biphenyl-4-yl)- | | (m) | (M + H)⁻ |
| pyridine-2- | 4-boronic acid | thieno[2,3-c] | | | |
| carboxylic | | pyridine-2- | | | |
| acid amide | | carboxylic acid | | | |
| (A, C) | | amide | | | |
| 4-Bromo- | 4- | 4-[4-Tetrahydro- | 187 | 2.49 min | 353.4 |
| thieno[2,3-c] | Hydroxyphenyl- | pyran-2-yloxy)- | | (m) | (M + H)⁻ |
| pyridine-2- | boronic acid | phenyl]-thieno[2,3-c] | | | |
| carboxylic | thp ether | pyridine-2- | | | |
| acid amide | | carboxylic acid | | | |
| (A, C) | | amide | | | |
| 4-Bromo- | 4- | 4-(4-Benzyloxy- | 188 | 2.72 min | 359.4 |
| thieno[2,3-c] | Benzyloxyphenyl- | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | (2- | 4-(2-Benzyloxy- | 189 | 2.54 min | 359.4 |
| thieno[2,3-c] | Benzyloxyphenyl) | phenyl)- | | (m) | (M + H)⁻ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 4′-(4,4,5,5- | 4-(4- | 190 | 1.69 min | 310.4 |
| thieno[2,3-c] | Tetramethyl- | Acetylamino- | | (m) | (M + H)⁻ |
| pyridine-2- | 1,3,2- | phenyl)- | | | |
| carboxylic | diaxaborolan- | thieno[2,3-c] | | | |
| acid amide | 2- | pyridine-2- | | | |
| (A, C) | yl)acetanilide | carboxylic acid | | | |
| | | amide | | | |
| 4-Bromo- | 1- | 4-(1- | 191 | 2.68 min | 432.5 |
| thieno[2,3-c] | (Phenylsulfonyl)- | Benzenesulfonyl- | | (m) | (M + H)⁻ |
| pyridine-2- | 1h-indol-3- | 1H-indol-3-yl)- | | | |
| carboxylic | ylboronic acid | thieno[2,3-c] | | | |
| acid amide | | pyridine-2- | | | |
| (A, C) | | carboxylic acid | | | |
| | | amide | | | |
| 4-Bromo-2- | 3- | 3-[2-(1H- | 192 | 1.36 min | 293.3 |
| (1H-tetrazol- | Aminophenyl- | Tetrazol-5-yl)- | | (m) | (M + H)⁻ |
| 5-yl)- | boronic acid | thieno[2,3-c] | | | |
| thieno[2,3-c] | hemisulfate | pyridine-4-yl]- | | | |
| pyridine | | phenylamine | | | |
| (A, C, F, G) | | | | | |
| 4-Bromo-2- | 3- | 2-(1H-Tetrazol-5- | 193 | 1.83 min | 346.3 |
| (1H-tetrazol- | (Trifluoromethyl) | yl)-4-(3-trifluoro- | | (m) | (M + H)⁻ |
| 5-yl)- | phenylboronic | methyl-phenyl)- | | | |
| thieno[2,3-c] | acid | thieno[2,3-c] | | | |
| pyridine | | pyridine | | | |
| (A, C, F, G) | | | | | |
| 4-Bromo-2- | 3,5- | 4-(3,5-Dichloro- | 194 | 1.91 min | 347.2 |
| (1H-tetrazol- | Dichlorophenyl- | phenyl)-2-1H- | | (m) | (M + H)⁻ |
| 5-yl)- | boronic acid | tetrazol- | | | |
| thieno[2,3-c] | | 5-yl)- | | | |
| pyridine | | thieno[2,3-c] | | | |
| (A, C, F, G) | | pyridine | | | |
| 4-Bromo-2- | 3,4- | 4-(3,4- | 195 | 1.54 min | 338.4 |
| (1H-tetrazol- | Dimethoxyphenyl- | Dimethoxy- | | (m) | (M + H)⁻ |
| 5-yl)- | boronic acid | phenyl)-2-(1H- | | | |
| thieno[2,3-c] | | tetrazol-5-yl)- | | | |
| pyridine | | thieno[2,3-c] | | | |
| (A, C, F, G) | | pyridine | | | |
| 4-Bromo-2- | 5-Fluoro-2- | 4-(5-Fluoro-2- | 196 | 1.65 min | 326.3 |
| (1H-tetrazol- | methoxyphenyl- | methoxy-phenyl)- | | (m) | (M + H)⁻ |
| 5-yl)- | boronic acid | 2-(1H- | | | |
| thieno[2,3-c] | | tetrazol-5-yl)- | | | |
| pyridine | | thieno[2,3-c] | | | |
| (A, C, F, G) | | pyridine | | | |
| 4-Bromo-2- | 4- | 2-(1H-Tetrazol-5- | 197 | 1.90 min | 362.3 |
| (1H-tetrazol- | (Trifluoromethoxy) | yl)-4-(4-trifluoro- | | (m) | (M + H)⁻ |
| 5-yl)- | benzeneboronic | methoxy-phenyl)- | | | |
| thieno[2,3-c] | acid | thieno[2,3-c] | | | |
| pyridine | | pyridine | | | |
| (A, C, F, G) | | | | | |
| 4-Bromo-2- | 4- | {4-[2-(1H- | 198 | 1.36 min | 308.4 |
| (1H-tetrazol- | (Hydroxymethyl) | Tetrazol-5-yl)- | | (m) | (M + H)⁻ |
| 5-yl)- | phenylboronic | thieno[2,3-c] | | | |
| thieno[2,3-c] | acid | pyridin-4-yl]- | | | |
| pyridine | | phenyl}-methanol | | | |
| (A, C, F, G) | | | | | |
| 4-Bromo-2- | 4′(4,4,5,5- | N-{4-[2-(1H- | 199 | 1.39 min | 335.4 |
| (1H-tetrazol- | Tetramethyl- | Tetrazol-5-yl)- | | (m) | (M + H)⁻ |
| 5-yl)- | 1,3,2- | thieno[2,3-c] | | | |
| thieno[2,3-c] | dioxaborolan-2- | pyridin-4-yl]- | | | |
| pyridine | yl)acetanilide | phenyl}-acetamide | | | |
| (A, C, F, G) | | | | | |
| 4-Bromo-2- | 1- | 4-Naphthalen-1- | 200 | 1.82 min | 328.4 |
| (1H-tetrazol- | Naphthalene- | yl-2-(1H-tetrazol- | | (m) | (M + H)⁻ |
| 5-yl)- | boronic acid | 5-yl)-thieno[2,3-c] | | | |
| thieno[2,3-c] | | pyridine | | | |
| pyridine | | | | | |
| (A, C, F, G) | | | | | |
| 4-Bromo-2- | 4- | 4-[2-(1H- | 201 | 1.37 min | 294.3 |
| (1H-tetrazol- | Hydroxyphenyl- | Tetrazol-5-yl)- | | (m) | (M + H)⁻ |
| 5-yl)- | boronic acid | thieno[2,3-c] | | | |
| thieno[2,3-c] | | pyridin-4-yl]- | | | |
| pyridine | | phenol | | | |
| (A, C, F, G) | | | | | |
| 4-Bromo-2- | 3- | 4-(3-Isopropyl- | 202 | 1.90 min | 320.4 |
| (1H-tetrazol- | Isopropylphenyl- | phenyl)-2-(1H- | | (m) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 3- | 3-[2-(1H- | 203 | 1.43 min | 294.3 |
| (1H-tetrazol- | Hydroxyphenyl- | Tetrazol-5-yl)- | | (m) | (M + H)⁻ |
| 5-yl)- | boronic acid | thieno[2,3-c] | | | |
| thieno[2,3-c] | | pyridin-4-yl]- | | | |
| pyridine | | phenol | | | |
| (A, C, F, G) | | | | | |
| 4-Bromo-2- | (2- | 4-(2-Benzyloxy- | 204 | 1.88 min | 384.5 |
| (1H-tetrazol- | Benzyloxyphenyl) | phenyl)-2-(1H- | | (m) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | Pyridine-4- | 4-Pyridin-4-yl-2- | 205 | 1.26 min | 279.3 |
| (1H-tetrazol- | boronic acid | (1H-tetrazol-5-yl)- | | (m) | (M + H)⁻ |
| 5-yl)- | | thieno[2,3-c] | | | |
| thieno[2,3-c] | | pyridine | | | |
| pyridine | | | | | |
| (A, C, F, G) | | | | | |
| 4-Bromo-2- | 1- | 4-(1- | 206 | 2.01 min | 457.5 |
| (1H-tetrazol- | (Phenylsulfonyl)- | Benzenesulfonyl- | | (l) | (M + H)⁻ |
| 5-yl)- | 1h-indol-3- | 1H-indol-3-yl)- | | | |
| thieno[2,3-c] | ylboronic acid | 2-(1H-tetrazol-5- | | | |
| pyridine | | yl)-thieno[2,3-c] | | | |
| (A, C, F, G) | | pyridine | | | |
| 4-Bromo-2- | 3,4-Dihydro- | 4-(3,4-Dihydro- | 207 | 1.64 min | 350.4 |
| (1H-tetrazol- | 2h-1,5- | 2H- | | (l) | (M + H)⁻ |
| 5-yl)- | benzodioxepin-7- | benzo[b][1,4] | | | |
| thieno[2,3-c] | ylboronic acid | dioxepin-7-yl)-2-(1H- | | | |
| pyridine | | tetrazol-5-yl)- | | | |
| (A, C, F, G) | | thieno[2,3-c] | | | |
| | | pyridine | | | |
| 4-Bromo-2- | 4- | 4-Biphenyl-4-yl- | 208 | 2.00 min | 354.4 |
| (1H-tetrazol- | Biphenylboronic | 2-(1H-tetrazol-5-yl)- | | (l) | (M + H)⁻ |
| 5-yl)- | acid | thieno[2,3-c] | | | |
| thieno[2,3-c] | | pyridine | | | |
| pyridine | | | | | |
| (A, C, F, G) | | | | | |
| 4-Bromo-2- | 2- | 4-(2-Fluoro- | 209 | 2.03 min | 372.4 |
| (1H-tetrazol- | Fluorobiphenyl- | biphenyl-4-yl)-2- | | (l) | (M + H)⁻ |
| 5-yl)- | 4-boronic | (1H-tetrazol-5-yl)- | | | |
| thieno[2,3-c] | acid | thieno[2,3-c] | | | |
| pyridine | | pyridine | | | |
| (A, C, F, G) | | | | | |
| 4-Bromo-2- | 4- | 1-{4-[2-(1H- | 210 | 1.55 min | 320.4 |
| (1H-tetrazol- | Acetylphenyl- | Tetrazol-5-yl)- | | (l) | (M + H)⁻ |
| 5-yl)- | boronic acid | thieno[2,3-c] | | | |
| thieno[2,3-c] | | pyridin-4- | | | |
| pyridine | | yl]-phenyl}- | | | |
| (A, C, F, G) | | ethanone | | | |
| 4-Bromo-2- | o- | 2-(1H-Tetrazol-5- | 211 | 1.63 min | 292.4 |
| (1H-tetrazol- | Tolyboronic | yl)-4-o-tolyl- | | (l) | (M + H)⁻ |
| 5-yl)- | acid | thieno[2,3-c] | | | |
| thieno[2,3-c] | | pyridine | | | |
| pyridine | | | | | |
| (A, C, F, G) | | | | | |
| 4-Bromo-2- | 2- | 4-(2-(Chloro- | 212 | 1.66 min | 312.8 |
| (1H-tetrazol- | Chlorophenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 3,4- | 4- | 213 | 1.59 min | 322.3 |
| (1H-tetrazol- | Methylenedioxy- | Benzo[1,3]dioxol- | | (l) | (M + H)⁻ |
| 5-yl)- | phenylboronic acid | 5-yl-2-(1H-tetrazol- | | | |
| thieno[2,3-c] | | 5-yl)-thieno[2,3-c] | | | |
| pyridine | | pyridine | | | |
| (A, C, F, G) | | | | | |
| 4-Bromo-2- | 4- | 4-(4-Methoxy- | 214 | 1.60 min | 308.4 |
| (1H-tetrazol- | Methoxyphenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 2- | 2-(1H-Tetrazol-5- | 215 | 1.77 min | 362.3 |
| (1H-tetrazol- | (Trifluoromethoxy) | yl)-4-(2-trifluoro- | | (l) | (M + H)⁻ |
| 5-yl)- | benzeneboronic | methoxy-phenyl)- | | | |
| thieno[2,3-c] | acid | thieno[2,3-c] | | | |
| pyridine | | pyridine | | | |
| (A, C, F, G) | | | | | |
| 4-Bromo-2- | 2,3-Dihydro- | 4-(2,3-Dihydro- | 216 | 1.61 min | 320.4 |
| (1H-tetrazol- | 1-benzofuran- | benzofuran-5-yl)- | | (l) | (M + H)⁻ |
| 5-yl)- | 5-ylboronic | 2-(1H-tetrazol-5-yl)- | | | |
| thieno[2,3-c] | acid | thieno[2,3-c] | | | |
| pyridine | | pyridine | | | |
| (A, C, F, G) | | | | | |
| 4-Bromo-2- | 4- | 2-(1H-Tetrazol-5- | 217 | 1.70 min | 292.4 |
| (1H-tetrazol- | Methylphenyl- | yl)-4-p-tolyl- | | (l) | (M + H)⁻ |
| 5-yl)- | boronic acid | thieno[2,3-c] | | | |
| thieno[2,3-c] | | pyridine | | | |
| pyridine | | | | | |
| (A, C, F, G) | | | | | |
| 4-Bromo-2- | (3- | {3-[2-(1H- | 218 | 1.38 min | 308.4 |
| (1H-tetrazol- | Hydroxymethyl- | Tetrazol-5-yl)- | | (l) | (M + H)⁻ |
| 5-yl)- | phenyl)boronic | thieno[2,3-c] | | | |
| thieno[2,3-c] | acid | pyridin-4-yl]- | | | |
| pyridine | | phenyl}-methanol | | | |
| (A, C, F, G) | | | | | |
| 4-Bromo-2- | 3- | 2-(1H-Tetrazol-5- | 219 | 1.86 min | 362.3 |
| (1H-tetrazol- | (Trifluoromethoxy) | yl)-4-(3-trifluoro- | | (l) | (M + H)⁻ |
| 5-yl)- | benzeneboronic | methoxy-phenyl)- | | | |
| thieno[2,3-c] | acid | thieno[2,3-c] | | | |
| pyridine | | pyridine | | | |
| (A, C, F, G) | | | | | |
| 4-Bromo-2- | 2- | 4-Biphenyl-2-yl- | 220 | 1.85 min | 354.4 |
| (1H-tetrazol- | Biphenyl- | 2-(1H-tetrazol-5-yl)- | | (l) | (M + H)⁻ |
| 5-yl)- | boronic acid | thieno[2,3-c] | | | |
| thieno[2,3-c] | | pyridine | | | |
| pyridine | | | | | |
| (A, C, F, G) | | | | | |
| 4-Bromo-2- | (2- | 4-(2-Phenoxy- | 221 | 1.88 min | 370.4 |
| (1H-tetrazol- | Phenoxy)phenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 3- | 4-(3-Nitro- | 222 | 1.64 min | 323.3 |
| (1H-tetrazol- | Nitrophenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 4- | 4-[2-(1H- | 223 | 1.31 min | 293.3 |
| (1H-tetrazol- | Aminophenyl- | Tetrazol-5-yl)- | | (l) | (M + H)⁻ |
| 5-yl)- | boronic acid | thieno[2,3-c] | | | |
| thieno[2,3-c] | | pyridin-4-yl]- | | | |
| pyridine | | phenylamine | | | |
| (A, C, F, G) | | | | | |
| 4-Bromo-2- | 8- | 8-[2-(1H- | 224 | 1.47 min | 329.4 |
| (1H-tetrazol- | Quinoline- | Tetrazol-5-yl)- | | (l) | (M + H)⁻ |
| 5-yl)- | boronic acid | thieno[2,3-c] | | | |
| thieno[2,3-c] | | pyridin-4-yl]- | | | |
| pyridine | | quinoline | | | |
| (A, C, F, G) | | | | | |
| 4-Bromo-2- | 4- | 4-(4-Chloro- | 225 | 1.74 min | 312.8 |
| (1H-tetrazol- | Chlorophenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 3- | 4-(3-Ethoxy- | 226 | 1.73 min | 322.4 |
| (1H-tetrazol- | Ethoxyphenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 4- | 4-(4- | 227 | 1.73 min | 324.4 |
| (1H-tetrazol- | (Methylthio) | Methylsulfanyl- | | (l) | (M + H)⁻ |
| 5-yl)- | phenylboronic | phenyl)-2-(1H- | | | |
| thieno[2,3-c] | acid | tetrazol-5-yl)- | | | |
| pyridine | | thieno[2,3-c] | | | |
| (A, C, F, G) | | pyridine | | | |
| 4-Bromo-2- | 4- | 4-(4-Benzyloxy- | 228 | 1.97 min | 384.5 |
| (1H-tetrazol- | Benzyloxphenyl- | phenyl)-2-(1H- | | (l) | (M + H)⁻ |
| 5-yl)- | boronic acid | tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 2-Fluoro-3- | 4-(2-Fluoro-3- | 229 | 1.60 min | 326.3 |
| (1H-tetrazol- | methoxyphenyl- | methoxy-phenyl)- | | (l) | (M + H)⁻ |
| 5-yl)- | boronic acid | 2-(1H-tetrazol-5-yl)- | | | |
| thieno[2,3-c] | | thieno[2,3-c] | | | |
| pyridine | | pyridine | | | |
| (A, C, F, G) | | | | | |
| 4-Bromo-2- | 4- | 4-[4-(Tetrahydro- | 230 | 1.83 min | 378.4 |
| (1H-tetrazol- | Hydroxyphenyl- | pyran-2-yloxy)- | | (l) | (M + H)⁻ |
| 5-yl)- | boronic acid | phenyl]-2-(1H- | | | |
| thieno[2,3-c] | thp ether | tetrazol-5-yl)- | | | |
| pyridine | | thieno[2,3-c] | | | |
| (A, C, F, G) | | pyridine | | | |
| 4-Bromo-2- | Phenylboronic | 4-Phenyl-2-(1H- | 231 | 1.55 min | 278.3 |
| (1H-tetrazol- | acid | tetrazol-5-yl)- | | (l) | (M + H)⁻ |
| 5-yl)- | | thieno[2,3-c] | | | |
| thieno[2,3-c] | | pyridine | | | |
| pyridine | | | | | |
| (A, C, F, G) | | | | | |
| 4-Bromo- | (1,1′- | 4-Biphenyl-4-yl- | 232 | 10.48 min | 331.3 |
| thieno[2,3-c] | Biphenyl-4- | thieno[2,3-c] | | (a) | (M + H)⁺ |
| pyridine-2- | yl)-boronic | pyridine-2- | | | |
| carboxylic | acid | carboxylic acid | | | |
| acid amide | | amide | | | |
| (A, C) | | | | | |
| 4-Bromo- | Phenylboronic | 4-Phenyl- | 233 | 8.42 min | 255.2 |
| thieno[2,3-c] | acid | thieno[2,3-c] | | (a) | (M + H)⁺ |
| pyridine-2- | | pyridine-2- | | | |
| carboxylic | | carboxylic acid | | | |
| acid amide | | amide | | | |
| (A, C) | | | | | |
| 4-Bromo- | trans-2- | 4-Styryl- | 234 | 9.38 min | 281.2 |
| thieno[2,3-c] | (4,4,5,5- | thieno[2,3-c] | | (a) | (M + H)⁺ |
| pyridine-2- | tetramethyl- | pyridine-2- | | | |
| carboxylic | 1,3,2- | carboxylic acid | | | |
| acid amide | dioxaborolan- | amide | | | |
| (A, C) | 2-yl)styrene | | | | |
| 4-Bromo- | Furan-3- | 4-Furan-3-yl- | 235 | 7.66 min | 245.2 |
| thieno[2,3-c] | boronic acid | thieno[2,3-c] | | (a) | (M + H)⁺ |
| pyridine-2- | | pyridine-2- | | | |
| carboxylic | | carboxylic acid | | | |
| acid amide | | amide | | | |
| (A, C) | | | | | |
| 4-Bromo- | [2-Chloro-4- | 4-(4-Amino-3- | 236 | 8.13 min | 304.3 |
| thieno[2,3-c] | (4,4,5,5- | chloro-phenyl)- | | (a) | (M + H)⁺ |
| pyridine-2- | tetramethyl- | thieno[2,3-c] | | | |
| carboxylic | [1,3,2]dioxaborolan- | pyridine-2- | | | |
| acid amide | 2-yl)-phenyl]- | carboxylic acid | | | |
| (A, C) | carbamic acid | amide | | | |
| 4-Bromo- | 3- | 4-(3-Amino- | 237 | 7.24 min | 270.3 |
| thieno[2,3-c] | Aminobenzene- | phenyl)- | | (a) | (M + H)⁺ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 2- | 4-(3-Fluoro- | 238 | 10.63 min | 349.3 |
| thieno[2,3-c] | Fluorobiphenyl- | biphenyl-4-yl)- | | (a) | (M + H)⁺ |
| pyridine-2- | 4-boronic | thieno[2,3-c] | | | |
| carboxylic | acid | pyridine-2- | | | |
| acid amide | | carboxylic acid | | | |
| (A, C) | | amide | | | |
| 4-Bromo- | 3- | 4-Thiophen-3-yl- | 239 | 8.15 min | 261.1 |
| thieno[2,3-c] | Thiophene- | thieno[2,3-c] | | (a) | (M + H)⁺ |
| pyridine-2- | boronic acid | pyridine-2- | | | |
| carboxylic | | carboxylic acid | | | |
| acid amide | | amide | | | |
| (A, C) | | | | | |
| 4-Bromo- | Biphenyl-3- | 4-Biphenyl-3-yl- | 240 | 10.37 min | 331.3 |
| thieno[2,3-c] | boronic acid | thieno[2,3-c] | | (a) | (M + H)⁺ |
| pyridine-2- | | pyridine-2- | | | |
| carboxylic | | carboxylic acid | | | |
| acid amide | | amide | | | |
| (A, C) | | | | | |
| 4-Bromo- | 4- | 4-(4-Formyl- | 241 | 7.85 min | 283.2 |
| thieno[2,3-c] | Formylphenyl- | phenyl)- | | (a) | (M + H)⁺ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | (3- | 3-(2-Carbamoyl- | 242 | 6.32 min | 297.1 |
| thieno[2,3-c] | Carboxyphenyl) | thieno[2,3-c] | | (a) | (M − H)⁺ |
| pyridine-2- | boronic acid | pyridin-4-yl)- | | | |
| carboxylic | | benzoic acid | | | |
| acid amide | | | | | |
| (A, C) | | | | | |
| 4-Bromo- | 3-Formylphenyl- | 4-(3-Formyl- | 243 | 7.83 min | 283.8 |
| thieno[2,3-c] | boronic acid | phenyl)- | | (a) | (M + H)⁺ |
| pyridine-2- | | thieno[2,3-c] | | | |
| carboxylic | | pyridine-2- | | | |
| acid amide | | carboxylic acid | | | |
| (A, C) | | amide | | | |
| 4-Bromo- | 3- | 4-(3-Cyano- | 244 | 8.15 min | 280.2 |
| thieno[2,3-c] | Cyanophenyl- | phenyl)- | | (a) | (M + H)⁺ |
| pyridine-2- | boronic acid | thieno[2,3-c] | | | |
| carboxylic | | pyridine-2- | | | |
| acid amide | | carbozylic acid | | | |
| (A, C) | | amide | | | |
| 4-Bromo- | 2- | 4-Naphthalen-2- | 245 | 9.78 min | 303.1 |
| thieno[2,3-c] | Naphthalene- | yl-thieno[2,3-c] | | (a) | (M − H)⁺ |
| pyridine-2- | boronic acid | pyridine-2- | | | |
| carboxylic | | carboxylic acid | | | |
| acid amide | | amide | | | |
| (A, C) | | | | | |
| 4-Bromo- | 4- | 4-(4-Phenoxy- | 246 | 10.45 min | 347.3 |
| thieno[2,3-c] | Phenoxybenzene- | phenyl)- | | (a) | (M + H)⁺ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | (3- | 4-(3-Benzyloxy- | 247 | 10.42 | 361.2 |
| thieno[2,3-c] | Benzyloxyphenyl) | phenyl)- | | (a) | (M + H)⁺ |
| pyridine-2- | boronic acid | thieno[2,3-c] | | | |
| carboxylic | | pyridine-2- | | | |
| acid amide | | carboxylic acid | | | |
| (A, C) | | amide | | | |
| 4-Bromo- | 3- | 4-Biphenyl-3-yl- | 248 | 8.06 min | 332.3 |
| thieno[2,3-c] | Biphenyl- | thieno[2,3-c] | | (a) | (M + H)⁺ |
| pyridine-2- | boronic acid | pyridine-2- | | | |
| carboxylic | | carboxylic acid | | | |
| acid methyl | | | | | |
| ester | | | | | |
| (A, B) | | | | | |
| 4-(4-Iodo- | 4-Chloro- | 4-(4′-Chloro- | 249 | 8.78 min | 380, 382 |
| phenoxy)- | phenylboronic | biphenyl-4- | | (b) | (M − H)⁻ |
| thieno[2,3-c] | acid | yloxy)- | | | (i) |
| pyridine-2- | | thieno[2,3-c] | | | |
| carboxylic | | pyridine-2- | | | |
| acid | | carboxylic acid | | | |
| (A, D, E) | | | | | |
| 4-Bromo- | 4-(4,4,5,5- | 4-(1H-Pyrazol-4- | 250 | 9.65 min | 302.0 |
| thieno [2,3-c] | Tetramethyl- | yl)-thieno[2,3-c] | | (a) | (M + H)⁺ |
| pyridine-2- | [1,3,2]dioxaborolan- | pyridine-2- | | | |
| carboxylic | 2-yl)-1H-pyrazole | carboxylic acid | | | |
| acid tert- | | tert-butyl acid | | | |
| butyl ester | | | | | |
| (A, B) | | | | | |
| 4-bromo-1H- | 3-Biphenyl- | 4-Biphenyl-3-yl- | 251 | 7.55 min | 315.2 |
| pyrrolo[2,3-c] | boronic acid | 1H-pyrrolo[2,3-c] | | (a) | (M + H)⁺ |
| pyridine-2- | | pyridine-2- | | | |
| carboxylic | | carboxylic acid | | | |
| acid methyl | | | | | |
| ester (A, Z, | | | | | |
| AA) | | | | | |
| 4-Bromo- | 3-Chloro-4- | 4-(3-Chloro-4- | 252 | 2.72 min | 358 |
| thieno[2,3-c] | ethoxyphenyl- | ethoxy-phenyl)-2- | | (m) | (M + H)⁺ |
| pyridine-2- | boronic acid | (1H-tetrazol-5- | | | |
| carboxylic | | yl)-thieno[2,3-c] | | | |
| acid amide | | pyridine | | | |
| (A, C) | | | | | |
| 4-Bromo- | (5-Chloro-2- | 4-(5-Chloro-2- | 253 | 2.64 min | 328 |
| thieno[2,3-c] | methylphenyl) | methyl-phenyl)-2- | | (m) | (M + H)⁺ |
| pyridine-2- | boronic acid | (1H-tetrazol-5- | | | |
| carboxylic | | yl)-thieno[2,3-c] | | | |
| acid amide | | pyridine | | | |
| (A, C) | | | | | |
| 4-Bromo- | 5-Chloro-2- | 4-(5-Chloro-2- | 254 | 2.69 min | 358 |
| thieno[2,3-c] | ethoxyphenyl- | ethoxy-phenyl)-2- | | (m) | (M + H)⁺ |
| pyridine-2- | boronic acid | (1H-tetrazol-5- | | | |
| carboxylic | | yl)-thieno[2,3-c] | | | |
| acid amide | | pyridine | | | |
| (A, C) | | | | | |
| 4-Bromo- | 3-Chloro-4- | 4-(3-Chloro-4- | 255 | 3.05 min | 328 |
| thieno[2,3-c] | methylphenyl- | methyl-phenyl)-2- | | (m) | (M + H)⁺ |
| pyridine-2- | boronic acid | (1H-tetrazol-5- | | | |
| carboxylic | | yl)-thieno[2,3-c] | | | |
| acid amide | | pyridine | | | |
| (A, C) | | | | | |
| 4-Bromo- | 5-Chloro-2- | 4-Chloro-2-[2- | 256 | 2.34 min | 342 |
| thieno[2,3-c] | formylphenyl- | (1H-tetrazol-5- | | (m) | (M + H)⁺ |
| pyridine-2- | boronic acid | yl)-thieno[2,3-c] | | | |
| carboxylic | | pyridin-4-yl]- | | | |
| acid amide | | benzaldehyde | | | |
| (A, C) | | | | | |
| 4-Bromo-2- | 3- | 3-[2-(1H- | 257 | 6.29 (a) | 295 |
| (2H-tetrazol- | Aminophenyl- | Tetrazol-5-yl)- | | | (M + H)⁺ |
| 5-yl)- | boronic acid | thieno[2,3-c] | | | |
| thieno[2,3-c] | monohydrate | pyridin-4-yl]- | | | |
| pyridine | | phenylamine | | | |
| (A, C, F, G) | | | | | |
| 4-Bromo-2- | 4-(4,4,5,5- | 4-[2-(1H- | 258 | 6.06 (a) | 295 |
| (2H-tetrazol- | Tetramethyl- | Tetrazol-5-yl)- | | | (M + H)⁺ |
| 5-yl)- | [1,3,2]dioxaborolan- | thieno[2,3-c] | | | |
| thieno[2,3-c] | 2-yl)-phenylamine | pyridin-4-yl]- | | | |
| pyridine | | phenylamine | | | |
| (A, C, F, G) | | | | | |
| 4-Bromo-2- | (5-Methyl- | (5-Methyl- | 259 | 8.31 (a) | 426 |
| (2H-tetrazol- | benzooxazol- | benzooxazol-2- | | | (M + H)⁺ |
| 5-yl)- | 2-yl)-[4- | yl)-{4-[2-(2H- | | | |
| thieno[2,3-c] | (4,4,5,5- | tetrazol-5-yl)- | | | |
| pyridine | tetramethyl- | thieno[2,3-c] | | | |
| (A, C, F, G) | [1,3,2]dioxaborolan- | pyridin-4-yl]- | | | |
| | 2-yl)-phenyl]- | phenyl}-amine | | | |
| | amine | | | | |
| 4-Bromo-7- | 4-Biphenyl-3- | none | 259 | 4.78 min | 380 |
| chloro- | yl-7-chloro- | | A | (i) | (M + H)⁺ |
| thieno[2,3-c] | thieno[2,3-c] | | | | |
| pyridine-2- | pyridine-2- | | | | |
| carboxylic | carboxylic | | | | |
| acid methyl | acid methyl | | | | |
| ester | ester | | | | |
| 4-Bromo-1H- | Benzene- | 4-Phenyl-1H- | 259 | 5.91 min | 239.0 |
| pyrrolo[2,3-c] | boronic acid | pyrrolo[2,3-c] | B | (a) | (M + H)⁺ |
| pyridine-2- | | pyridine-2- | | | |
| carboxylic | | carboxylic acid | | | |
| acid methyl | | | | | |
| ester | | | | | |
| ( ) | | | | | |
| 4-Bromo-1H- | N- | 4-(1-Methyl-1H- | 259 | 6.53 min | 290.1 |
| pyrrolo[2,3-c] | Methylindole- | indol-5-yl)-1H- | C | (a) | (M + H)⁻ |
| pyridine-2- | 5-boronic acid | pyrrolo[2,3-c] | | | |
| carboxylic | | pyridine-2- | | | |
| acid methyl | | carboxylic acid | | | |
| ester | | | | | |
| ( ) | | | | | |
| 4-(3-Chloro- | 4- | 4-(4′-Methoxy- | 259 | 6.81 min | 362.2 |
| phenyl)- | Methoxyphenyl- | biphenyl-3-yl)- | D | (b) | (M + H)⁺ |
| thieno[2,3-c] | boronic acid | thieno[2,3-c] | | | |
| pyridine-2- | | pyridine-2- | | | |
| carboxylic | | carboxylic acid | | | |
| acid (A, B, E) | | | | | |
| 4-(4-Iodo- | 2-(4-Chloro- | 4-(4′-Chloro- | 259 | 8.78 min | 380, 382 |
| phenoxy)- | phenyl)- | biphenyl-4- | E | (a) | (M − H)⁻ |
| thieno[2,3-c] | 4,4,5,5- | yloxy)- | | | |
| pyridine-2- | tetramethyl- | thieno[2,3-c] | | | |
| carboxylic | [1,3,2]dioxa- | pyridine-2- | | | |
| acid (A, D, | borolane | carboxylic acid | | | |
| E) | | | | | |
| 4-(4-Bromo- | Benzothiophene- | 4-(4- | 259 | 7.47 min | 426.1 |
| phenoxy)-2- | 3-boronic acid | Benzo[b]thiophen- | F | (b) | (M − H)⁻ |
| (1H-tetrazol- | | 3-yl-phenoxy)-2- | | | |
| 5-yl)- | | (1H-tetrazol-5- | | | |
| thieno[2,3-c] | | yl)-thieno[2,3-c] | | | |
| pyridine | | pyridine | | | |
| (A, D, F, G) | | | | | |
| 4-(4-Bromo- | 2,3,4- | 2-(1H-Tetrazol- | 259 | 5.09 min | 460 |
| phenoxy)-2- | Trimethoxyphenyl- | 5-yl)-4-(2′,3′,4′- | G | (t) | (M − H)⁻ |
| (1H-tetrazol- | boronic acid | trimethoxy- | | | |
| 5-yl)- | | biphenyl-4- | | | |
| thieno[2,3-c] | | yloxy)- | | | |
| pyridine | | thieno[2,3-c] | | | |
| (A, D, F, G) | | pyridine | | | |
| 7-Amino-4- | 3- | 7-Amino-4-(3- | 259 | 5.21 min | 302, 304 |
| bromo- | Chlorophenyl- | chloro-phenyl)- | H | (t) | (M − H)⁻ |
| thieno[2,3-c] | boronic acid | thieno[2,3-c] | | | |
| pyridine-2- | | pyridine-2- | | | |
| carboxylic | | carboxylic acid | | | |
| acid amide | | amide | | | |
| (A-877887.0) | | | | | |
| 7-Amino-4- | 4- | 7-Amino-4-(4′- | 259 | 6.09 min | 360.3 |
| (3-chloro- | Methylphenyl- | methyl-biphenyl- | I | (t) | (M + H)⁺ |
| phenyl)- | boronic acid | 3-yl)-thieno[2,3-c] | | | |
| thieno[2,3-c] | | pyridine-2- | | | |
| pyridine-2- | | carboxylic acid | | | |
| carboxylic | | amide | | | |
| acid amide | | | | | |
| 7-Amino-4- | 3- | 7-Amino-4-(3′- | 259 | 5.53 min | 371.3 |
| (3-chloro- | Cyanophenyl- | cyano-biphenyl- | J | (t) | (M + H)⁺ |
| phenyl)- | boronic acid | 3-yl)-thieno[2,3-c] | | | |
| thieno[2,3-c] | | pyridine-2- | | | |
| pyridine-2- | | carboxylic acid | | | |
| carboxylic | | amide | | | |
| acid amide | | | | | |
| 7-Amino-4- | 2- | 7-Amino-4-(2′- | 259 | 5.67 min | 371.3 |
| (3-chloro- | Cyanophenyl- | cyano-biphenyl- | K | (t) | (M + H)⁺ |
| phenyl)- | boronic acid | 3-yl)-thieno[2,3-c] | | | |
| thieno[2,3-c] | | pyridine-2- | | | |
| pyridine-2- | | carboxylic acid | | | |
| carboxylic | | amide | | | |
| acid amide | | | | | |
| 7-Amino-4- | 2- | 7-Amino-4-(2′- | 259 | 6.00 min | 360.3 |
| (3-chloro- | Methylphenyl- | methyl-biphenyl- | L | (t) | (M + H)⁺ |
| phenyl)- | boronic acid | 3-yl)-thieno[2,3-c] | | | |
| thieno[2,3-c] | | pyridine-2- | | | |
| pyridine-2- | | carboxylic acid | | | |
| carboxylic | | amide | | | |
| acid amide | | | | | |
| 4-(4-Bromo- | 4- | 4-(4′-Nitro- | 259 | 9.91 (a) | 391 |
| phenylamino)- | Nitrophenyl- | biphenyl-4- | M | | (M + H)⁺ |
| thieno[2,3-c] | boronic acid | ylamino)- | | | |
| pyridine-2- | | thieno[2,3-c] | | | |
| carboxylic | | pyridine-2- | | | |
| acid amide | | carboxylic acid | | | |
| | | amide | | | |
| 4-(4-Bromo- | 4′-(4,4,5,5- | 4-(4′- | 259 | 8.04 (a) | 403 |
| phenylamino)- | Tetramethyl- | Acetylamino- | N | | (M + H)⁺ |
| thieno[2,3-c] | 1,3,2- | biphenyl-4- | | | |
| pyridine-2- | dioxaborolan-2- | ylamino)- | | | |
| carboxylic | yl)acetanilide | thieno[2,3-c] | | | |
| acid amide | | pyridine-2- | | | |
| | | carboxylic acid | | | |
| | | amide | | | |
| 4-(4-Bromo- | 4- | 4-(4′-Acetyl- | 259 | 5.36 (t) | 388 |
| phenylamino)- | Acetyphenyl- | biphenyl-4- | O | | (M + H)⁺ |
| thieno[2,3-c] | boronic acid | ylamino)- | | | |
| pyridine-2- | | thieno[2,3-c] | | | |
| carbonitrile | | pyridine-2- | | | |
| | | carboxylic acid | | | |
| | | amide | | | |
| 4-(4-Bromo- | 4-(N,N- | 4-(4′- | 259 | 10.36 (a) | 389 |
| phenylamino)- | dimethylamino) | Dimethylamino- | P | | (M + H)⁺ |
| thieno[2,3-c] | phenyl- | biphenyl-4- | | | |
| pyridine-2- | boronic acid | ylamino)- | | | |
| carboxylic | | thieno[2,3-c] | | | |
| acid amide | | pyridine-2- | | | |
| | | carboxylic acid | | | |
| | | amide | | | |
| 4-(4-Bromo- | 4′-(4,4,5,5- | 4-(4′-Amino- | 259 | 8.27 (a) | 361 |
| phenylamino)- | Tetramethyl- | biphenyl-4- | Q | | (M + H)⁺ |
| thieno[2,3-c] | 1,3,2- | ylamino)- | | | |
| pyridine-2- | dioxaborolan- | thieno[2,3-c] | | | |
| carboxylic | 2-yl)aniline | pyridine-2- | | | |
| acid amide | | carboxylic acid | | | |
| | | amide | | | |
| 4-(4-Bromo- | 4- | 4-(4′- | 259 | 8.41 (a) | 424 |
| phenylamino)- | (Methanesupfonyl) | Methanesulfonyl- | R | | (M + H)⁺ |
| thieno[2,3-c] | phenyl- | biphenyl-4- | | | |
| pyridine-2- | boronic acid | ylamino)- | | | |
| carboxylic | | thieno[2,3-c] | | | |
| acid amide | | pyridine-2- | | | |
| | | carboxylic acid | | | |
| | | amide | | | |
| 4-(4-Bromo- | 4- | 4-(4′-Fluoro- | 259 | 10.14 (a) | 364 |
| phenylamino)- | Fluorophenyl- | biphenyl-4- | S | | (M + H)⁺ |
| thieno[2,3-c] | boronic acid | ylamino)- | | | |
| pyridine-2- | | thieno[2,3-c] | | | |
| carboxylic | | pyridine-2- | | | |
| acid amide | | carboxylic acid | | | |
| | | amide | | | |
| 4-(4-Bromo- | 4- | 4-(4′- | 259 | 9.61 (a) | 390 |
| phenylamino)- | Methoxymethyl- | Methoxymethyl- | T | | (M + H)⁺ |
| thieno[2,3-c] | phenyl- | biphenyl-4- | | | |
| pyridine-2- | boronic acid | ylamino)- | | | |
| carboxylic | | thieno[2,3-c] | | | |
| acid amide | | pyridine-2- | | | |
| | | carboxylic acid | | | |
| | | amide | | | |
| 4-(4-Bromo- | 2-Methyl-4- | 4-(4′-Cyano-3′- | 259 | 9.75 (a) | 385 |
| phenylamino)- | (4,4,5,5- | methyl-biphenyl- | U | | (M + H)⁺ |
| thieno[2,3-c] | tetramethyl- | 4-ylamino)- | | | |
| pyridine-2- | [1,3,2]dioxaborolan- | thieno[2,3-c] | | | |
| carboxylic | 2-yl)- | pyridine-2- | | | |
| acid amide | benzonitrile | carboxylic acid | | | |
| | | amide | | | |
| 4-(4-Bromo- | Benzamide-4- | 4-(4′-Carbamoyl | 259 | 4.47 (t) | 389 |
| phenylamino)- | boronic acid | biphenyl-4- | V | | (M + H)⁺ |
| thieno[2,3-c] | | ylamino)- | | | |
| pyridine-2- | | thieno[2,3-c] | | | |
| carboxylic | | pyridine-2- | | | |
| acid amide | | carboxylic acid | | | |
| | | amide | | | |
| 4-(4-Bromo- | 4- | 4′-(2-Carbamoyl- | 259 | 7.43 (a) | 390 |
| phenylamino)- | Carboxyphenyl- | thieno[2,3-c] | W | | (M + H)⁺ |
| thieno[2,3-c] | boronic acid | pyridin-4- | | | |
| pyridine-2- | | ylamino)- | | | |
| carboxylic | | biphenyl-4- | | | |
| acid amide | | carboxylic acid | | | |
| 4-(4′- | 4,4,5,5- | 4-(4′-Ethynyl- | 259 | 5.89 (t) | 370 |
| Ethynyl- | Tetramethyl- | biphenyl-4- | X | | (M + H)⁺ |
| biphenyl-4- | 2-(4- | ylamino)- | | | |
| ylamino)- | trimethylsilanyl- | thieno[2,3-c] | | | |
| thieno[2,3-c] | ethynyl- | pyridine-2- | | | |
| pyridine-2- | phenyl)- | carboxylic acid | | | |
| carboxylic | [1,3,2]dioxa- | amide | | | |
| acid amide | borolane | | | | |
| 4-(4-Bromo- | 2-Fluoro-4- | 4-(4′-Cyano-3′- | 259 | 5.65 (t) | 389 |
| phenylamino)- | (4,4,5,5- | fluoro-biphenyl- | Y | | (M + H)⁺ |
| thieno[2,3-c] | tetramethyl- | 4-ylamino)- | | | |
| pyridine-2- | [1,3,2]dioxa- | thieno[2,3-c] | | | |
| carboxylic | borolan-2-yl)- | pyridine-2- | | | |
| acid amide | benzonitrile | carboxylic acid | | | |
| | | amide | | | |
| 8-Bromo- | 3- | 8-(Biphenyl-4- | 259 | 1.24 (a) | 325 |
| [1,6]naphthyr- | Biphenyl- | ylamino)- | Z | | (M − H)− |
| idine-2- | boronic acid | [1,6]naphthyridine- | | | |
| carboxylic | | 2-carboxylic | | | |
| acid | | acid | | | |
| 7-Amino-4- | 3- | 7-Amino-4-(3- | 259 | 2.90 (a) | 285.3 |
| bromo- | Aminophenyl- | amino-phenyl)- | AA | | (M + H)+; |
| thieno[2,3-c] | boronic acid | thieno[2,3-c] | | | 283.2 |
| pyridine-2- | | pyridine-2- | | | (M − H)− |
| carboxylic | | carboxylic acid | | | |
| acid amide | | amide | | | |
| (Entry BB2) | | | | | |
| 7-Amino-4- | 2-Phenyl-N- | 7-Amino-4-(4- | 259 | 1.39 (a) | 403.3 |
| bromo- | [4-(4,4,5,5- | phenylacetylamino- | BB | | (M + H)+ |
| thieno[2,3-c] | tetramethyl- | phenyl)- | | | |
| pyridine-2- | [1,3,2]dioxa- | thieno[2,3-c] | | | |
| carboxylic | borolan-2-yl)- | pyridine-2- | | | |
| acid amide | phenyl]- | carboxylic acid | | | |
| (Entry BB2) | acetamide | amide | | | |
| 7-Amino-4- | 1-Phenyl-3-[4- | 7-Amino-4-[4-(3- | 259 | 1.39 (a) | 404 |
| bromo- | (4,4,5,5- | phenyl-ureido)- | CC | | (M + H)+; |
| thieno[2,3-c] | tetramethyl- | phenyl]- | | | 402 |
| pyridine-2- | [1,3,2]dioxa- | thieno[2,3-c] | | | (M − H)− |
| carboxylic | borolan-2-yl)- | pyridine-2- | | | |
| acid amide | phenyl]-urea | carboxylic acid | | | |
| | | amide | | | |
| 7-Amino-4- | N-[3-(boronic | 7-Amino-4-(3- | 259 | 1.38 (a) | 389 |
| bromo- | acid)-phenyl]- | benzoylamino- | DD | | (M + H)+; |
| thieno[2,3-c] | benzamide | phenyl)- | | | 387 |
| pyridine-2- | | thieno[2,3-c] | | | (M − H)− |
| carboxylic | | pyridine-2- | | | |
| acid amide | | carboxylic acid | | | |
| | | amide | | | |
| 7-Amino-4- | 4-(4,4,5,5- | 7-Amino-4-(4- | 259 | 0.62 (a) | 285 |
| bromo- | Tetramethyl- | amino-phenyl)- | EE | | (M + H)+ |
| thieno[2,3-c] | [1,3,2]dioxa- | thieno[2,3-c] | | | |
| pyridine-2- | borolan-2-yl)- | pyridine-2- | | | |
| carboxylic | phenylamine | carboxylic acid | | | |
| acid amide | | amide | | | |
| 7-Amino-4- | Benzooxazol- | 7-Amino-4-[4- | 259 | 1.54 (a) | 402 |
| bromo- | 2-yl-[4- | (benzooxazol-2- | FF | | (M + H)+; |
| thieno[2,3-c] | (4,4,5,5- | ylamino)-phenyl]- | | | 400 |
| pyridine-2- | tetramethyl- | thieno[2,3-c] | | | (M − H)− |
| carboxylic | [1,3,2]dioxa- | pyridine-2- | | | |
| acid amide | borolan-2-yl)- | carboxylic acid | | | |
| | phenyl]- | amide | | | |
| | amine | | | | |

Other compounds obtained using general procedure L are shown (Table 9).

TABLE 9
Examples synthesized using general procedure L

| | Sulfonyl | | | | |
|---|---|---|---|---|---|
| | Chloride | | | HPLC Rₜ | |
| Aniline Precursor | Precursor | Product | Ex # | (Method) | m/z |
| 4-(3-Amino- | m- | 4-[3-(Toluene-3- | 260 | 9.10 min | 424 |
| phenyl)thieno[2,3- | Toluenesulfonyl | sulfonylamino)- | | (a) | (M + H)⁺ |
| c]pyridine-2- | chloride | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 2-Chloro-4- | 4-[3-(2-Chloro-4- | 261 | 10.05 min | 512 |
| phenyl)thieno[2,3- | trifluoromethyl | trifluoromethyl- | | (a) | (M + H)⁺ |
| c]pyridine-2- | sulfonyl | benzenesulfonylamino)- | | | |
| carboxylic acid | chloride | phenyl]- | | | |
| amide | | thieno[2,3- | | | |
| (A, C, J) | | c]pyridine-2- | | | |
| | | carboxylic acid | | | |
| | | amide | | | |
| 4-(3-Amino- | 2- | 4-[3-(Thiophene-2- | 262 | 8.57 min | 416 |
| phenyl)thieno[2,3- | Thiophensulfonyl | sulfonylamino)- | | (a) | (M + H)⁺ |
| c]pyridine-2- | Chloride | phenyl]-thieno[2,3- | | | |
| carboxylic acid | | c]pyridine-2- | | | |
| amide | | carboxylic acid | | | |
| (A, C, J) | | amide | | | |
| 4-[2-(2H-Tetrazol- | m- | 3-Methyl-N-{4-[2- | 263 | 7.74 min | 449 |
| 5-yl)-thieno[2,3- | Toluenesulfonyl | (2H-tetrazol-5-yl)- | | (a) | (M + H)⁺ |
| c]pyridin-4-yl]- | Chloride | thieno[2,3- | | | |
| phenylamine | | c]pyridin-4-yl]- | | | |
| (A, C, F, G, J) | | phenyl}- | | | |
| | | benzenesulfonamide | | | |
| 4-(4-Amino- | m- | 4-[4-(Toluene-3- | 264 | 9.15 min | 424 |
| phenyl)thieno[2,3- | Toluensulfonyl | sulfonylamino)- | | (a) | (M + H)+ |
| c]pyridine-2- | chloride | phenyl]-thieno[2,3- | | | |
| carboxylic acid | | c]pyridine-2- | | | |
| amide | | carboxylic acid | | | |
| (A, C, J) | | amide | | | |
| 4-(4-Amino- | Benzenesulfonyl | 4-(4- | 265 | 8.90 min | 410 |
| phenyl)thieno[2,3- | chloride | Benzenesulfonylamino- | | (a) | (M + H)+ |
| c]pyridine-2- | | phenyl)- | | | |
| carboxylic acid | | thieno[2,3- | | | |
| amide | | c]pyridine-2- | | | |
| (A, C, J) | | carboxylic acid | | | |
| | | amide | | | |
| 4-(Piperidin-4- | Benzenesulfonyl | 4-(1- | 265A | 8.34 (a) | 417.13 |
| ylamino)- | chloride | Benzenesulfonyl- | | | (M + H)+ |
| thieno[2,3- | | piperidin-4-yl | | | |
| c]pyridine-2- | | amino)-thieno[2,3- | | | |
| carboxylic acid | | c]pyridine-2-carboxylic | | | |
| amide | | acid amide | | | |
| 4- | Methanesulfonyl | N-(4- | 265B | 5.43 min | 393.0 |
| [2,2′]Bithiophenyl- | chloride | [2,2′]Bithiophenyl- | | (t) | (M + H)⁺ |
| 5-yl-thieno[2,3- | | 5-yl-thieno[2,3- | | | |
| c]pyridin-2- | | c]pyridin-2-yl)- | | | |
| ylamine (A, B, E, J, | | methanesulfonamide | | | |
| Q, R) | | | | | |
| 4- | Trifluoromethanesulfonyl | N-(4- | 265C | 6.25 min | 445 |
| [2,2′]Bithiophenyl- | chloride | [2,2′]Bithiophenyl- | | (t) | (M − H)⁻ |
| 5-yl-thieno[2,3- | | 5-yl-thieno[2,3- | | | |
| c]pyridin-2- | | c]pyridin-2-yl)- | | | |
| ylamine (A, B, E, J, | | 2,2,2-trifluoro- | | | |
| Q, R) | | acetamide | | | |

Other compounds obtained using general procedure M are shown (Table 10).

TABLE 10
Examples synthesized using general procedure M

| | Aldehyde | | | HPLC Rₜ | |
|---|---|---|---|---|---|
| Aniline Precursor | Precursor | Product | Ex # | (Method) | m/z |
| 4-(3-Amino- | Cyclopropane | 4-[3- | 266 | 9.51 min | 324 |
| phenyl)thieno[2,3- | Carboxaldehyde | (Cyclopropylmethyl- | | (a) | (M + H)⁺ |
| c]pyridine-2- | | amino)-phenyl]- | | | |
| carboxylic acid | | thieno[2,3- | | | |
| amide | | c]pyridine-2- | | | |
| (A, C, J) | | carboxylic acid | | | |
| | | amide | | | |
| 4-(3-Amino- | Tert-butyl N- | {2-[3-(2- | 267 | 9.25 min | 413 |
| phenyl)thieno[2,3- | (2- | Carbamoyl- | | (a) | (M + H)⁺ |
| c]pyridine-2- | oxoethyl)carbamate | thieno[2,3- | | | |
| carboxylic acid | | c]pyridin-4-yl)- | | | |
| amide | | phenylamino]- | | | |
| (A, C, J) | | ethyl}-carbamic | | | |
| | | acid tert-butyl ester | | | |
| 4-(3-Amino- | 4-Pyridine | 4-{3-[(Pyridin-4- | 268 | 7.79 min | 361 |
| phenyl)thieno[2,3- | carboxaldehyde | ylmethyl)-amino]- | | (a) | (M + H)⁺ |
| c]pyridine-2- | | phenyl}-thieno[2,3- | | | |
| carboxylic acid | | c]pyridine-2- | | | |
| amide | | carboxylic acid | | | |
| (A, C, J) | | amide | | | |
| 4-(3-Amino- | Benzaldehyde | 4-(3-Benzylamino- | 269 | 9.90 min | 360 |
| phenyl)thieno[2,3- | | phenyl)-thieno[2,3- | | (a) | (M + H)⁺ |
| c]pyridine-2- | | c]pyridine-2- | | | |
| carboxylic acid | | carboxylic acid | | | |
| amide | | amide | | | |
| (A, C, J) | | | | | |
| 3-[2-(2H-Tetrazol- | Cyclopropane | Cyclopropylmethyl- | 270 | 7.78 min | 349 |
| 5-yl)-thieno[2,3- | Carboxaldehyde | {3-[2-(1H- | | (a) | (M + H)⁺ |
| c]pyridin-4-yl]- | | tetrazol-5-yl)- | | | |
| phenylamine | | thieno[2,3- | | | |
| (A, C, F, G, J) | | c]pyridin-4-yl]- | | | |
| | | phenyl}-amine | | | |
| 4-[2-(2H-Tetrazol- | Cyclopropane | Cyclopropylmethyl- | 271 | 7.77 min | 349 |
| 5-yl)-thieno[2,3- | Carboxaldehylde | {4-[2-(1H-tetrazol- | | (a) | (M + H)⁺ |
| c]pyridin-4-yl]- | | 5-yl)-thieno[2,3- | | | |
| phenylamine | | c]pyridin-4-yl]- | | | |
| (A, C, F, G, J) | | phenyl}-amine | | | |
| 4-[2-(2H-Tetrazol- | 2-Pyridine | Pyridin-2-ylmethyl- | 272 | 6.97 min | 386 |
| 5-yl)-thieno[2,3- | carboxaldehyde | {3-[2-(1H-tetrazol- | | (a) | (M + H)⁺ |
| c]pyridin-4-yl]- | | 5-yl)-thieno[2,3- | | | |
| phenylamine | | c]pyridin-4-yl]- | | | |
| (A, C, F, G, J) | | phenyl}-amine | | | |
| 4-[2-(2H-Tetrazol- | 3-Pyridine | Pyridin-3-ylmethyl- | 273 | 6.88 min | 386 |
| 5-yl)-thieno[2,3- | carboxaldehyde | {3-[2-(1H-tetrazol- | | (a) | (M + H)⁺ |
| c]pyridin-4-yl]- | | 5-yl)-thieno[2,3- | | | |
| phenylamine | | c]pyridin-4-yl]- | | | |
| (A, C, F, G, J) | | phenyl}-amine | | | |
| 4-(3-Amino- | Phenyl- | 4-(3- | 274 | 10.41 min | 374 |
| phenyl)thieno[2,3- | acetaldehyde | Phenethylamino- | | (a) | (M + H)⁺ |
| c]pyridine-2- | | phenyl)-thieno[2,3- | | | |
| carboxylic acid | | c]pyridine-2- | | | |
| amide | | carboxylic acid | | | |
| (A, C, J) | | amide | | | |
| 4-(3-Amino- | 3-Phenyl- | 4-[3-(3-Phenyl- | 275 | 10.89 min | 388 |
| phenyl)thieno[2,3- | propionaldehyde | propylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 2-Methoxy- | 4-[3-(2-Methoxy- | 276 | 10.02 min | 390 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 3-Methoxy- | 4-[3-(3-Methoxy- | 277 | 9.83 min | 390 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 4-Methoxy- | 4-[3-(4-Methoxy- | 278 | 9.83 min | 390 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 2-Chloro- | 4-[3-(2-Chloro- | 279 | 10.47 min | 394 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 3-Chloro- | 4-[3-(3-Chloro- | 280 | 10.44 min | 394 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 4-Chloro- | 4-[3-(4-Chloro- | 281 | 10.52 min | 394 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 2- | 4-[3-(2- | 282 | 10.64 min | 428 |
| phenyl)thieno[2,3- | Trifluoromethyl- | Trifluoromethyl- | | (a) | (M + H)⁺ |
| c]pyridine-2- | benzaldehyde | benzylamino)- | | | |
| carboxylic acid | | phenyl]-thieno[2,3- | | | |
| amide | | c]pyridine-2- | | | |
| (A, C, J) | | carboxylic acid | | | |
| | | amide | | | |
| 4-(3-Amino- | 3- | 4-[3-(2- | 283 | 10.60 min | 428 |
| phenyl)thieno[2,3- | Trifluoromethyl- | Trifluoromethyl- | | (a) | (M + H)⁺ |
| c]pyridine-2- | benzaldehyde | benzylamino)- | | | |
| carboxylic acid | | phenyl]-thieno[2,3- | | | |
| amide | | c]pyridine-2- | | | |
| (A, C, J) | | carboxylic acid | | | |
| | | amide | | | |
| 4-(3-Amino- | 4- | 4-[3-(2- | 284 | 10.67 min | 428 |
| phenyl)thieno[2,3- | Trifluoromethyl- | Trifluoromethyl- | | (a) | (M + H)⁺ |
| c]pyridine-2- | benzaldehyde | benzylamino)- | | | |
| carboxylic acid | | phenyl]-thieno[2,3- | | | |
| amide | | c]pyridine-2- | | | |
| (A, C, J) | | carboxylic acid | | | |
| | | amide | | | |
| 4-(3-Amino- | 2,4- | 4-[3-(2,4- | 285 | 9.99 min | 420 |
| phenyl)thieno[2,3- | Dimethoxy- | Dimethoxy- | | (a) | (M + H)⁺ |
| c]pyridine-2- | benzaldehyde | benzylamino)- | | | |
| carboxylic acid | | phenyl]-thieno[2,3- | | | |
| amide | | c]pyridine-2- | | | |
| (A, C, J) | | carboxylic acid | | | |
| | | amide | | | |
| 4-(3-Amino- | 2,4-Dichloro- | 4-[3-(2,4-Dichloro- | 286 | 11.30 min | 429 |
| phenyl)thieno[2,3- | benzaldehyde | benzylamino)- | | (a) | (M + H)⁺ |
| c]pyridine-2- | | phenyl]-thieno[2,3- | | | |
| carboxylic acid | | c]pyridine-2- | | | |
| amide | | carboxylic acid | | | |
| (A, C, J) | | amide | | | |
| 4-(3-Amino- | 2,3-Dihydro- | 4-{3-[(2,3- | 287 | 9.61 min | 418 |
| phenyl)thieno[2,3- | benzo[1,4]dioxine- | Dihydro- | | (a) | (M + H)⁺ |
| c]pyridine-2- | 6- | benzo[1,4]dioxin- | | | |
| carboxylic acid | carbaldehyde | 6-ylmethyl)- | | | |
| amide | | amino]-phenyl}- | | | |
| (A, C, J) | | thieno[2,3- | | | |
| | | c]pyridine-2- | | | |
| | | carboxylic acid | | | |
| | | amide | | | |

Other compounds obtained using general procedure N are shown (Table 11).

TABLE 11
Examples synthesized using general procedure N

| | Isocyanate | | | HPLC Rₜ | |
|---|---|---|---|---|---|
| Aniline Precursor | Precursor | Product | Ex # | (Method) | m/z |
| 4-(3-Amino- | Cyclopentyl | 4-[3-(3- | 288 | 8.64 min (a) | 381 |
| phenyl)thieno[2,3- | isocyanate | Cyclopentyl- | | | (M + H)⁺ |
| c]pyridine-2- | | ureido)-phenyl]- | | | |
| carboxylic acid | | thieno[2,3- | | | |
| amide | | c]pyridine-2- | | | |
| (A, C, J) | | carboxylic acid | | | |
| | | amide | | | |
| 4-(3-Amino- | m-Tolyl | 4-[3-(3-m-Tolyl- | 289 | 9.46 min (a) | 403 |
| phenyl)thieno[2,3- | isocyanate | ureido)-phenyl]- | | | (M + H)⁺ |
| c]pyridine-2- | | thieno[2,3- | | | |
| carboxylic acid | | c]pyridine-2- | | | |
| amide | | carboxylic acid | | | |
| (A, C, J) | | amide | | | |
| 4-(3-Amino- | 2-Fluoro-5- | 4-{3-[3-(2-Fluoro- | 290 | 10.35 min (a) | 474 |
| phenyl)thieno[2,3- | trifluoromethyl) | 5-trifluoromethyl- | | | (M + H)⁺ |
| c]pyridine-2- | phenyl | phenyl)-ureido]- | | | |
| carboxylic acid | isocyanate | phenyl}-thieno[2,3- | | | |
| amide | | c]pyridine-2- | | | |
| (A, C, J) | | carboxylic acid | | | |
| | | amide | | | |
| 3-[2-(2H-Tetrazol- | m-Tolyl | 1-{3-[2-(2H- | 291 | 8.05 min (a) | 428 |
| 5-yl)-thieno[2,3- | isocyanate | Tetrazol-5-yl)- | | | (M + H)⁺ |
| c]pyridin-4-yl]- | | thieno[2,3- | | | |
| phenylamine | | c]pyridin-4-yl]- | | | |
| (A, C, F, G, J) | | phenyl}-3-m-tolyl- | | | |
| | | urea | | | |
| 4-[2-(2H-Tetrazol- | m-Tolyl | 1-{4-[2-(2H- | 292 | 7.99 min (a) | 428 |
| 5-yl)-thieno[2,3- | isocyanate | Tetrazol-5-yl)- | | | (M + H)⁺ |
| c]pyridin-4-yl]- | | thieno[2,3- | | | |
| phenylamine | | c]pyridin-4-yl]- | | | |
| (A, C, F, G, J) | | phenyl}-3-m-tolyl- | | | |
| | | urea | | | |
| 4-[2-(2H-Tetrazol- | Phenyl | 1-Phenyl-3-{4-[2- | 293 | 7.60 min (a) | 414 |
| 5-yl)-thieno[2,3- | isocyanate | (2H-tetrazol-5-yl)- | | | (M + H)⁺ |
| c]pyridin-4-yl]- | | thieno[2,3- | | | |
| phenylamine | | c]pyridin-4-yl]- | | | |
| (A, C, F, G, J) | | phenyl}-urea | | | |
| 4-[2-(2H-Tetrazol- | o-Tolyl | 1-{4-[2-(2H- | 294 | 7.75 min (a) | 428 |
| 5-yl)-thieno[2,3- | isocyanate | Tetrazol-5-yl)- | | | (M + H)⁺ |
| c]pyridin-4-yl]- | | thieno[2,3- | | | |
| phenylamine | | c]pyridin-4-yl]- | | | |
| (A, C, F, G, J) | | phenyl}-3-o-tolyl- | | | |
| | | urea | | | |
| 4-[2-(2H-Tetrazol- | p-Tolyl | 1-{4-[2-(2H- | 295 | 7.95 min (a) | 428 |
| 5-yl)-thieno[2,3- | isocyanate | Tetrazol-5-yl)- | | | (M + H)⁺ |
| c]pyridin-4-yl]- | | thieno[2,3- | | | |
| phenylamine | | c]pyridin-4-yl]- | | | |
| (A, C, F, G, J) | | phenyl}-3-p-tolyl- | | | |
| | | urea | | | |
| 4-[2-(2H-Tetrazol- | 1-Isocyanato- | 1-{4-[2-(2H- | 296 | 8.15 min (a) | 482 |
| 5-yl)-thieno[2,3- | 2- | Tetrazol-5-yl)- | | | (M + H)⁺ |
| c]pyridin-4-yl]- | trifluoromethyl- | thieno[2,3- | | | |
| phenylamine | benzene | c]pyridin-4-yl]- | | | |
| (A, C, F, G, J) | | phenyl}-3-(2- | | | |
| | | trifluoromethyl- | | | |
| | | phenyl)-urea | | | |
| 4-[2-(2H-Tetrazol- | 1-Isocyanato- | 1-{4-[2-(2H- | 297 | 8.50 min (a) | 482 |
| 5-yl)-thieno[2,3- | 3- | Tetrazol-5-yl)- | | | (M + H)⁺ |
| c]pyridin-4-yl]- | trifluoromethyl- | thieno[2,3- | | | |
| phenylamine | benzene | c]pyridin-4-yl]- | | | |
| (A, C, F, G, J) | | phenyl}-3-(3- | | | |
| | | trifluoromethyl- | | | |
| | | phenyl)-urea | | | |
| 4-[2-(2H-Tetrazol- | 1-Isocyanato- | 1-{4-[2-(2H- | 298 | 8.58 min (a) | 482 |
| 5-yl)-thieno[2,3- | 4- | Tetrazol-5-yl)- | | | (M + H)⁺ |
| c]pyridin-4-yl]- | trifloromethyl- | thieno[2,3- | | | |
| phenylamine | benzene | c]pyridin-4-yl]- | | | |
| (A, C, F, G, J) | | phenyl}-3-(4- | | | |
| | | trifluoromethyl- | | | |
| | | phenyl)-urea | | | |
| 4-[2-(2H-Tetrazol- | 1,4-Dichloro- | 1-(2,5-Dichloro- | 299 | 8.83 min (a) | 483 |
| 5-yl)-thieno[2,3- | 2-isocyanato- | phenyl)-3-{4-[2- | | | (M + H)⁺ |
| c]pyridin-4-yl]- | benzene | (2H-tetrazol-5-yl)- | | | |
| phenylamine | | thieno[2,3- | | | |
| (A, C, F, G, J) | | c]pyridin-4-yl]- | | | |
| | | phenyl}-urea | | | |
| 4-[2-(2H-Tetrazol- | 1,3-Dichloro- | 1-(3,5-Dichloro- | 300 | 8.99 min (a) | 483 |
| 5-yl)-thieno[2,3- | 5-isocyanato- | phenyl)-3-{4-[2- | | | (M + H)⁺ |
| c]pyridin-4-yl]- | benzene | (2H-tetrazol-5-yl)- | | | |
| phenylamine | | thieno[2,3- | | | |
| (A, C, F, G, J) | | c]pyridin-4-yl]- | | | |
| | | phenyl}-urea | | | |
| 4-[2-(2H-Tetrazol- | 1,3-Dichloro- | 1-(2,6-Dichloro- | 301 | 7.65 min (a) | 483 |
| 5-yl)-thieno[2,3- | 2-isocyanato- | phenyl)-3-{4-[2- | | | (M + H)⁺ |
| c]pyridin-4-yl]- | benzene | (2H-tetrazol-5-yl)- | | | |
| phenylamine | | thieno[2,3- | | | |
| (A, C, F, G, J) | | c]pyridin-4-yl]- | | | |
| | | phenyl}-urea | | | |
| 4-(4-Amino- | Phenyl | 4-[4-(3-Phenyl- | 302 | 9.17 min (a) | 389 |
| phenyl)thieno[2,3- | isocyante | ureido)-phenyl]- | | | (M + H)⁺ |
| c]pyridine-2- | | thieno[2,3- | | | |
| carboxylic acid | | c]pyridine-2- | | | |
| amide | | carboxylic acid | | | |
| (A, C, J) | | amide | | | |
| 4-(4-Amino- | 1-Isocyanato- | 4-{4-[3-(3- | 303 | 10.10 min (a) | 457 |
| phenyl)thieno[2,3- | 3- | Trifluoromethyl- | | | (M + H)⁺ |
| c]pyridine-2 | trifluoromethyl- | phenyl)-ureido]- | | | |
| carboxylic acid | benzene | phenyl}-thieno[2,3- | | | |
| amide | | c]pyridine-2- | | | |
| (A, C, J) | | carboxylic acid | | | |
| | | amide | | | |
| 4-(4-Amino- | Isopropyl | 4-[4-(3-Isopropyl- | 304 | 7.90 min (a) | 355 |
| phenyl)thieno[2,3- | isocyanate | ureido)-phenyl]- | | | (M + H)⁺ |
| c]pyridine-2- | | thieno[2,3- | | | |
| carboxylic acid | | c]pyridine-2- | | | |
| amide | | carboxylic acid | | | |
| (A, C, J) | | amide | | | |
| 4-(Piperidin-4- | Isothiocyanato- | 4-(1- | 304A | 5.01 (a) | 412.1 |
| ylamino)- | benzene | Phenylthiocarbamoyl- | | | (M + H)+ |
| thieno[2,3- | | piperidin- | | | |
| c]pyridine-2- | | 4-ylamino)- | | | |
| carboxylic acid | | thieno[2,3- | | | |
| amide | | c]pyridine-2- | | | |
| | | carboxylic acid | | | |
| | | amide | | | |
| 4-(Piperidin-4- | Isocyanato- | 4-(1- | 304B | 4.72 (a) | 396.2 |
| ylamino)- | benzene | Phenylcarbamoyl- | | | (M + H)+ |
| thieno[2,3- | | piperidin-4-yl | | | |
| c]pyridine-2- | | amino)-thieno[2,3- | | | |
| carboxylic acid | | c]pyridine-2-carboxylic | | | |
| amide | | acid amide | | | |
| 4-(Piperidin-4- | 4-Isocyanato- | 4-[1-(3,4- | 304C | 4.48 (a) | 456.2 |
| ylamino)- | 1,2- | Dimethoxy- | | | (M + H)+ |
| thieno[2,3- | dimethoxy- | phenylcarbamoyl)- | | | |
| c]pyridine-2- | benzene | piperidin-4- | | | |
| carboxylic acid | | ylamino]- | | | |
| amide | | thieno[2,3-c | | | |
| | | ]pyridine-2- | | | |
| | | carboxylic acid | | | |
| | | amide | | | |
| 4- | Ethyl | 1-(4- | 304D | 6.25 min (t) | 384 |
| [2,2′]Bithiophenyl- | isocyanate | [2,2′]Bithiophenyl- | (M − H)⁻ | | |
| 5-yl-thieno[2,3- | | 5-yl-thieno[2,3- | | | |
| c]pyridin-2- | | c]pyridin-2-yl)-3- | | | |
| ylamine (A, B, E, J, | | ethyl-urea | | | |
| Q, R) | | | | | |
| 4-(4′-Methoxy- | Ethyl | 1-Ethyl-3-[4-(4′- | 304E | 6.05 min (t) | 404.2 |
| biphenyl-3-yl)- | isocyanate | methoxy-biphenyl- | | | (M + H)⁺ |
| thieno[2,3- | | 3-yl)-thieno[2,3- | | | |
| c]pyridin-2- | | c]pyridin-2-yl]-urea | | | |
| ylamine (A, B, E, J, | | | | | |
| Q, R) | | | | | |

Other compounds obtained using general procedure O are shown (Table 12).

TABLE 12
Examples synthesized using general procedure O

| | Acyl Chloride | | | HPLC Rₜ | |
|---|---|---|---|---|---|
| Aniline Precursor | Precursor | Product | Ex # | (Method) | m/z |
| 4-(3-Amino- | 2-Fluoro-4- | 4-[3-(2-Fluoro-4- | 305 | 10.03 min | 460 |
| phenyl)thieno[2,3- | trifluoromethylbenzoyl | trifluoromethyl- | | (a) | (M + H)⁺ |
| c]pyridine-2- | chloride | benzoylamino)- | | | |
| carboxylic acid | | phenyl]-thieno[2,3- | | | |
| amide | | c]pyridine-2- | | | |
| (A, C, J) | | carboxylic acid | | | |
| | | amide | | | |
| 4-(3-Amino- | m- | 4-[3-(3- | 306 | 10.01 min | 442 |
| phenyl)thieno[2,3- | (Trifluoromethyl) | Trifluoromethyl- | | (a) | (M + H)⁺ |
| c]pyridine-2- | benzoyl | benzoylamino)- | | | |
| carboxylic acid | chloride | phenyl]-thieno[2,3- | | | |
| amide | | c]pyridine-2- | | | |
| (A, C, J) | | carboxylic acid | | | |
| | | amide | | | |
| 4-[2-(2H-Tetrazol- | m- | N-{4-[2-(2H- | 307 | 8.42 min | 467 |
| 5-yl)-thieno[2,3- | (Trifluoromethyl) | Tetrazol-5-yl)- | | (a) | (M + H)⁺ |
| c]pyridin-4-yl]- | benzoyl | thieno[2,3- | | | |
| phenylamine | chloride | c]pyridin-4-yl]- | | | |
| (A, C, F, G, J) | | phenyl}-3- | | | |
| | | trifluoromethyl- | | | |
| | | benzamide | | | |
| 4-(3-Amino- | 2-Thiophene- | 4-{3-[(Thiophene- | 308 | 8.76 min | 380 |
| phenyl)thieno[2,3- | carbonyl | 2-carbonyl)- | | (a) | (M + H)⁺ |
| c]pyridine-2- | chloride | amino]-phenyl}- | | | |
| carboxylic acid | | thieno[2,3- | | | |
| amide | | c]pyridine-2- | | | |
| (A, C, J) | | carboxylic acid | | | |
| | | amide | | | |
| 4-(4-Amino- | m- | 4-[4-(3- | 309 | 9.96 min | 442 |
| phenyl)thieno[2,3- | (Trifluoromethyl) | Trifluoromethyl- | | (a) | (M + H)⁺ |
| c]pyridine-2- | benzoyl | benzoylamino)- | | | |
| carboxylic acid | chloride | phenyl]-thieno[2,3- | | | |
| amide | | c]pyridine-2- | | | |
| (A, C, J) | | carboxylic acid | | | |
| | | amide | | | |
| 4-(4-Amino- | Benzoyl | 4-(4- | 310 | 8.86 min | 374 |
| phenyl)thieno[2,3- | chloride | Benzoylamino- | | (a) | (M + H)⁺ |
| c]pyridine-2- | | phenyl)-thieno[2,3- | | | |
| carboxylic acid | | c]pyridine-2- | | | |
| amide | | carboxylic acid | | | |
| (A, C, J) | | amide | | | |
| 4-(4-Amino- | Isobutyryl | 4-(4- | 311 | 8.16 min | 340 |
| phenyl)thieno[2,3- | chloride | Isobutyrylamino- | | (a) | (M + H)⁺ |
| c]pyridine-2- | | phenyl)-thieno[2,3- | | | |
| carboxylic acid | | c]pyridine-2- | | | |
| amide | | carboxylic acid | | | |
| (A, C, J) | | amide | | | |
| 4-(4-Amino- | 3- | 4-[4-(3- | 312 | 9.96 min | 442 |
| phenyl)-thieno[2,3- | Trifluoromethyl- | Trifluoromethyl- | | (a) | |
| c]pyridine-2- | benzoyl | benzoylamino)- | | | |
| carboxylic acid | chloride | phenyl]-thieno[2,3- | | | |
| amide | | c]pyridine-2- | | | |
| (A, C, J) | | carboxylic acid | | | |
| | | amide | | | |
| 4-(4-Amino- | Benzoyl | 4-(4- | 313 | 8.86 min | 374 |
| phenyl)-thieno[2,3- | chloride | Benzoylamino- | | (a) | |
| c]pyridine-2- | | phenyl)-thieno[2,3- | | | |
| carboxylic acid | | c]pyridine-2- | | | |
| amide | | carboxylic acid | | | |
| (A, C, J) | | amide | | | |
| 4-(4-Amino- | Isobutyryl | 4-(4- | 314 | 8.16 min | 340 |
| phenyl)-thieno[2,3- | chloride | Isobutyrylamino- | | (a) | |
| c]pyridine-2- | | phenyl)-thieno[2,3- | | | |
| carboxylic acid | | c]pyridine-2- | | | |
| amide | | carboxylic acid | | | |
| (A, C, J) | | amide | | | |
| 4-(Biphenyl-4- | 3-Amino- | 4-(Biphenyl-4- | 314A | 5.78 min | 424.12 (M + H)⁺ |
| ylamino)- | propan-1-ol | ylamino)- | | (q) | |
| thieno[2,3- | | thieno[2,3- | | | |
| c]pyridine-2- | | c]pyridine-2- | | | |
| carboxylic acid | | carboxylic acid (3- | | | |
| (A, B, I, E) | | hydroxy-propyl)- | | | |
| | | amide | | | |
| 4-(2-Carbamoyl- | Pyridin-4- | 4-[4-(Pyridin-4- | 314B | 4.23 min | 390.1 (M + H)⁺ |
| thieno[2,3- | ylamine | ylcarbamoyl)- | | (q) | |
| c]pyridin-4- | | phenylamino]- | | | |
| ylamino)-benzoic | | thieno[2,3- | | | |
| acid (A, B, I, H) | | c]pyridine-2- | | | |
| | | carboxylic acid | | | |
| | | amide acetate salt | | | |
| 4-(2-Carbamoyl- | Piperidin-4- | 4-[4-(Piperidin-4- | 314C | 2.60 min | 396.09 (M + H)⁺ |
| thieno[2,3- | ylamine | ylcarbamoyl)- | | (j) | |
| c]pyridin-4- | | phenylamino]- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(Biphenyl-4- | Dimethylamine | 4-(Biphenyl-4- | 314D | 6.33 min | 374.3 (M + H)⁺ |
| ylamino)- | | ylamino)- | | (j) | |
| thieno[2,3- | | thieno[2,3- | | | |
| c]pyridine-2- | | c]pyridine-2- | | | |
| carboxylic acid | | carboxylic acid | | | |
| (A, B, I, E) | | dimethylamide | | | |
| 4-(Biphenyl-4- | Methylamine | 4-(Biphenyl-4- | 314E | 6.13 min | 360.3 (M + H)⁺ |
| ylamino)- | | ylamino)- | | (j) | |
| thieno[2,3- | | thieno[2,3- | | | |
| c]pyridine-2- | | c]pyridine-2- | | | |
| carboxylic acid | | carboxylic acid | | | |
| (A, B, I, E) | | methylamide | | | |
| 4-(2-Carbamoyl- | 4-Amino- | 4-[4-(4-Cyano- | 314F | 5.23 min | 414.19 (M + H)⁺ |
| thieno[2,3- | benzonitrile | phenylcarbamoyl)- | | (j) | |
| c]pyridin-4- | | phenylamino]- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | 3,4- | 4-[4-(3,4- | 314G | 4.91 min | 449.19 (M + H)⁺ |
| thieno[2,3- | Dimethoxy- | Dimethoxy- | | (j) | |
| c]pyridin-4- | phenylamine | phenylcarbamoyl)- | | | |
| ylamino)-benzoic | | phenylamino]- | | | |
| acid (A, B, I, H) | | thieno[2,3- | | | |
| | | c]pyridine-2- | | | |
| | | carboxylic acid | | | |
| | | amide | | | |
| 4-(2-Carbamoyl- | 3-Amino- | 4-[4-(3-Cyano- | 314H | 5.23 min | 414.2 (M + H)⁺ |
| thieno[2,3- | benzonitrile | phenylcarbamoyl)- | | (j) | |
| c]pyridin-4- | | phenylamino]- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | 4-Amino- | 4-[4-(4-Sulfamoyl- | 314I | 4.61 min | 468.07 (M + H)⁺ |
| thieno[2,3- | benzenesulfonamide | phenylcarbamoyl)- | | (j) | |
| c]pyridin-4- | | phenylamino]- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | Isopropylamine | 4-(4- | 314J | 7.28 min | 355.23 (M + H)⁺ |
| thieno[2,3- | | Isopropylcarbamoyl- | | (a) | |
| c]pyridin-4- | | phenylamino)- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | Benzylamine | 4-(4- | 314K | 8.18 min | 403.23 (M + H)⁺ |
| thieno[2,3- | | Benzylcarbamoyl- | | (a) | |
| c]pyridin-4- | | phenylamino)- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | 3-Amino- | 3-[4-2- | 314L | 8.22 min | 467.99 (M + H)⁺ |
| thieno[2,3- | azetidine-1- | Carbamoyl- | | (a) | |
| c]pyridin-4- | carboxylic | thieno[2,3- | | | |
| ylamino)-benzoic | acid tert-butyl | c]pyridin-4- | | | |
| acid (A, B, I, H) | ester | ylamino)- | | | |
| | | benzoylamino]- | | | |
| | | azetidine-1- | | | |
| | | carboxylic acid | | | |
| | | tert-butyl ester | | | |
| 4-(2-Carbamoyl- | Cyclohexylamine | 4-(4- | 314M | 8.44 min | 395.29 (M + H)⁺ |
| thieno[2,3- | | Cyclohexylcarbamoyl- | | (a) | |
| c]pyridin-4- | | phenylamino)- | | | |
| ylamino)-benzoic | | thieno[2,3- | | | |
| acid (A, B, I, H) | | c]pyridine-2- | | | |
| | | carboxylic acid | | | |
| | | amide | | | |
| 4-(2-Carbamoyl- | N,N- | 4-[4-(2- | 314M | 5.76 min | 384.08 (M + H)⁺ |
| thieno[2,3- | Dimethylethylenediamine | Dimethylamino- | | (a) | |
| c]pyridin-4- | | ethylcarbamoyl)- | | | |
| ylamino)-benzoic | | phenylamino]- | | | |
| acid (A, B, I, H) | | thieno[2,3- | | | |
| | | c]pyridine-2- | | | |
| | | carboxylic acid | | | |
| | | amide acetate salt | | | |
| 4-(2-Carbamoyl- | 2- | 4-[4-2- | 314N | 7.39 min | 387.2 (M + H)⁺ |
| thieno[2,3- | (Methylthio)ethylamine | Methylsulfanyl- | | (a) | |
| c]pyridin-4- | | ethylcarbamoyl)- | | | |
| ylamino)-benzoic | | phenylamino]- | | | |
| acid (A, B, I, H) | | thieno[2,3- | | | |
| | | c]pyridine-2- | | | |
| | | carboxylic acid | | | |
| | | amide | | | |
| 4-(2-Carbamoyl- | Methyl-(2- | 4-{4-[Methyl-(2- | 314O | 7.59 min | 401.1 (M + H)⁺ |
| thieno[2,3- | methylsulfanyl- | methylsulfanyl- | | (a) | |
| c]pyridin-4- | ethyl)-amine | ethyl)-carbamoyl]- | | | |
| ylamino)-benzoic | | phenylamino}- | | | |
| acid (A, B, I, H) | | thieno[2,3- | | | |
| | | c]pyridine-2- | | | |
| | | carboxylic acid | | | |
| | | amide acetate salt | | | |
| 4-(Biphenyl-4- | 3-Amino- | 4-(Biphenyl-4- | 314P | 5.78 min | 424.12 (M + H)⁺ |
| ylamino)- | propan-1-ol | ylamino)- | | (q) | |
| thieno[2,3- | | thieno[2,3- | | | |
| c]pyridine-2- | | c]pyridine-2- | | | |
| carboxylic acid | | carboxylic acid (3- | | | |
| (A, B, I, E) | | hydroxy-propyl)- | | | |
| | | amide | | | |
| 4-(Piperidin-4- | Benzoyl | 4-(1-Benzoyl- | 314Q | 7.30 (a) | 381.2 |
| ylamino)- | chloride | piperidin-4- | | | (M + H)+ |
| thieno[2,3- | | ylamino)-thieno | | | |
| c]pyridine-2- | | [2,3- | | | |
| carboxylic acid | | c]pyridine-2- | | | |
| amide | | carboxylic acid | | | |
| | | amide | | | |
| 4-(Piperidin-4- | Phenyl-acetyl | 4-[1-Phenylacetyl- | 314R | 7.60 (a) | 395.2 |
| ylamino)- | chloride | piperidin-4-ylamino)- | | | (M + H)+ |
| thieno[2,3- | | thieno[2,3- | | | |
| c]pyridine-2- | | c]pyridine-2- | | | |
| carboxylic acid | | carboxylic | | | |
| amide | | acid amide | | | |
| 4-(Piperidin-4- | 3-Phenyl- | 4-[1-(3-Phenyl- | 314S | 8.09 (a) | 409.2 |
| ylamino)- | propionyl | propionyl)- | | | (M + H)+ |
| thieno[2,3- | chloride | piperidin- | | | |
| c]pyridine-2- | | 4-ylamino]- | | | |
| carboxylic acid | | thieno[2,3- | | | |
| amide | | c]pyridine-2- | | | |
| | | carboxylic acid | | | |
| | | amide | | | |
| 4-(Piperidin-4- | 3-Cyano- | 4-[1-(3-Cyano- | 314T | 7.23 (a) | 406.2 |
| ylamino)- | benzoyl | benzoyl)-piperidin- | | | (M + H)+ |
| thieno[2,3- | chloride | 4- | | | |
| c]pyridine-2- | | ylamino]- | | | |
| carboxylic acid | | thieno[2,3- | | | |
| amide | | c]pyridine-2-carboxylic | | | |
| | | acid amide | | | |
| 4-(Piperidin-4- | 4-Methoxy- | 4-[1-(4-Methoxy- | 314U | 7.44 (a) | 411.1 |
| ylamino)- | benzoyl | benzoyl)-piperidin- | | | (M + H) |
| thieno[2,3- | chloride | 4-ylamino]- | | | |
| c]pyridine-2- | | thieno[2,3- | | | |
| carboxylic acid | | c]pyridine-2- | | | |
| amide | | carboxylic acid | | | |
| | | amide | | | |
| 4-(Piperidin-4- | 2-Methoxy- | 4-[1-(2-Methoxy- | 314V | 7.35 (a) | 411.1 |
| ylamino)- | benzoyl | benzoyl)-piperidin- | | | (M + H)+ |
| thieno[2,3- | chloride | 4-ylamino]- | | | |
| c]pyridine-2- | | thieno[2,3- | | | |
| carboxylic acid | | c]pyridine-2- | | | |
| amide | | carboxylic acid | | | |
| | | amide | | | |
| 4-(Piperidin-4- | Pyridine-2- | 4-[1-(Pyridine-2- | 314W | 6.24 (a) | 382.2 |
| ylamino)- | carbonyl | carbonyl)-piperidin- | | | (M + H) |
| thieno[2,3- | chloride | 4-ylamino]- | | | |
| c]pyridine-2- | | thieno[2,3- | | | |
| carboxylic acid | | c]pyridine- | | | |
| amide | | 2-carboxylic acid | | | |
| | | amide | | | |
| 4-(Piperidin-4- | Pyridine-3- | 4-[1-(Pyridine-3- | 314X | 6.08 (a) | 382.2 |
| ylamino)- | carbonyl | carbonyl)-piperidin- | | | (M + H) |
| thieno[2,3- | chloride | 4-ylamino]- | | | |
| c]pyridine-2- | | thieno[2,3- | | | |
| carboxylic acid | | c]pyridine- | | | |
| amide | | 2-carboxylic acid | | | |
| | | amide | | | |
| 4-(Piperidin-4- | Pyridine-4- | 4-[1-(Pyridine-4- | 314Y | 6.05 (a) | 382.2 |
| ylamino)- | carbonyl | carbonyl)-piperidin- | | | (M + H) |
| thieno[2,3- | chloride | 4-ylamino]- | | | |
| c]pyridine-2- | | thieno[2,3- | | | |
| carboxylic acid | | c]pyridine- | | | |
| amide | | 2-carboxylic acid | | | |
| | | amide | | | |
| 4-(Piperidin-4- | Dimethylamino- | 4-[1-(2- | 314Z | 5.12 (a) | 362.2 |
| ylamino)- | acetyl | Dimethylamino- | | | (M + H) |
| thieno[2,3- | chloride | acetyl)-piperidin- | | | |
| c]pyridine-2- | | 4-ylamino]- | | | |
| carboxylic acid | | thieno[2,3-c]pyridine- | | | |
| amide | | 2-carboxylic | | | |
| | | acid amide | | | |
| 4-(Piperidin-4- | Isoxazole-5- | 4-[1-(Isoxazole-5- | 314AA | 6.48 (a) | 372.2 |
| ylamino)- | carbonyl | carbonyl)-piperidin- | | | (M + H) |
| thieno[2,3- | chloride | 4-ylamino]- | | | |
| c]pyridine-2- | | thieno[2,3- | | | |
| carboxylic acid | | c]pyridine- | | | |
| amide | | 2-carboxylic acid | | | |
| | | amide | | | |
| 4-(Piperidin-4- | Benzo[1,3]dioxole- | 4-[1- | 314BB | 7.34 (a) | 425.2 |
| ylamino)- | 5- | (Benzo[1,3]dioxole- | | | (M + H) |
| thieno[2,3- | carbonyl | 5-carbonyl)- | | | |
| c]pyridine-2- | chloride | piperidin-4- | | | |
| carboxylic acid | | ylamino]- | | | |
| amide | | thieno[2,3-c] | | | |
| | | pyridine-2- | | | |
| | | carboxylic acid | | | |
| | | amide | | | |
| 4-(Piperidin-4- | Thiophene-2- | 4-[1-(Thiophene-2- | 314CC | 7.25 (a) | 387.2 |
| ylamino)- | carbonyl | carbonyl)-piperidin- | | | (M + H) |
| thieno[2,3- | chloride | 4-ylamino]- | | | |
| c]pyridine-2- | | thieno[2,3- | | | |
| carboxylic acid | | c]pyridine- | | | |
| amide | | 2-carboxylic acid | | | |
| | | amide | | | |
| 4-(Piperidin-4- | (2-Methoxy- | 4-{1-[2-(2- | 314DD | 6.03 (a) | 393.2 |
| ylamino)- | ethoxy)-acetyl | Methoxy-ethoxy)- | | | (M + H)+ |
| thieno[2,3- | chloride | acetyl]- | | | |
| c]pyridine-2- | | piperidin-4- | | | |
| carboxylic acid | | ylamino}- | | | |
| amide | | thieno[2,3-c]pyridine- | | | |
| | | 2- | | | |
| | | carboxylic acid | | | |
| | | amide | | | |
| 4-(Piperidin-4- | 3,5-Dimethyl- | 4-[1-(3,5- | 314EE | 6.67 (a) | 400.2 |
| ylamino)- | isoxazole-4- | Dimethyl- | | | (M + H) |
| thieno[2,3- | carbonyl chloride | isoxazole-4-carbonyl)- | | | |
| c]pyridine-2- | | piperidin-4- | | | |
| carboxylic acid | | ylaminol]-thieno[2, | | | |
| amide | | 3-c]pyridine-2- | | | |
| | | carboxylic acid amide | | | |
| 4-(Piperidin-4- | 3- | 4-[4-(2-Carbamoyl- | 314FF | 6.40 (a) | 391.2 |
| ylamino)- | Chlorocarbonyl- | thieno[2,3-c]pyridin- | | | (M + H) |
| thieno[2,3- | propionic | 4-ylamino)- | | | |
| c]pyridine-2- | acid methyl | piperidin-1-yl]-4- | | | |
| carboxylic acid | ester | ox | | | |
| amide | | o-butyric acid | | | |
| | | methyl ester | | | |
| 4-(Piperidin-4- | 1-Acetyl- | 4-[1-(1-Acetyl- | 314GG | 6.01 (a) | 430.3 |
| ylamino)- | piperidine-4- | piperidine-4- | | | (M + H) |
| thieno[2,3- | carbonyl chloride | carbonyl)- | | | |
| c]pyridine-2- | | piperidin-4- | | | |
| carboxylic acid | | ylamino]- | | | |
| amide | | thieno[2,3- | | | |
| | | c]pyridine-2- | | | |
| | | carboxylic acid | | | |
| | | amide | | | |
| 4-(Piperidin-4- | Phenylchloroformate | 4-(2-Carbamoyl- | 314HH | 5.21 (a) | 397.2 |
| ylamino)- | | thieno[2,3- | | | (M + H)+ |
| thieno[2,3- | | c]pyridin-4- | | | |
| c]pyridine-2- | | ylamino)- | | | |
| carboxylic acid | | piperidine-1- | | | |
| amide | | carboxylic | | | |
| | | acid phenyl ester | | | |
| 4-(Piperidin-4- | Trifluoromethylacetylchloride | 4-[1-(2,2,2- | 314II | 7.69 (a) | 400.2 |
| ylamino)- | | Trifluoro-acetyl)- | | | (M + H) |
| thieno[2,3- | | piperidin- | | | |
| c]pyridine-2- | | 4-ylamino]- | | | |
| carboxylic acid | | thieno[2,3-c]pyridine- | | | |
| amide | | 2-carboxylic | | | |
| | | acid amide | | | |
| 4- | Acetic | N-(4- | 314JJ | 6.37 min | 355 (M − H)− |
| [2,2′]Bithiophenyl- | anhydride | [2,2′]Bithiophenyl- | | (t) | |
| 5-yl-thieno[2,3- | | 5-yl-thieno[2,3- | | | |
| c]pyridin-2- | | c]pyridin-2-yl)- | | | |
| ylamine (A, B, E, J, | | acetamide | | | |
| Q, R) | | | | | |
| 4- | Methoxy- | N-(4- | 314KK | 6.70 min | 385 (M − H)− |
| [2,2]Bithiophenyl- | acetyl | [2,2′]Bithiophenyl- | | (t) | |
| 5-yl-thieno[2,3- | chloride | 5-yl-thieno[2,3- | | | |
| c]pyridin-2- | | c]pyridin-2-yl)-2- | | | |
| ylamine (A, B, E, J, | | methoxy-acetamide | | | |
| Q, R) | | | | | |
| 4- | Trifluoroacetic | N-[4- | 314LL | 6.47 min | 505 (M − H)− |
| [2,2′]Bithiophenyl- | anhydride | [2,2′]Bithiophenyl- | | (t) | |
| 5-yl-thieno[2,3- | | 5-yl-3-(2,2,2- | | | |
| c]pyridin-2- | | trifluoro-acetyl)- | | | |
| ylamine (A, B, E, J, | | thieno[2,3- | | | |
| Q, R) | | c]pyridin-2-yl]- | | | |
| | | 2,2,2-trifluoro- | | | |
| | | acetamide | | | |
| 4-(4′-Methoxy- | Acetic | N-[4-(4′-Methoxy- | 314MM | 6.24 min | 375 (M + H)⁺ |
| biphenyl-3-yl)- | anhydride | biphenyl-3-yl)- | | (t) | |
| thieno[2,3- | | thieno[2,3- | | | |
| c]pyridin-2- | | c]pyridin-2-yl]- | | | |
| ylamine (A, B, E, | | acetamide | | | |
| J) | | | | | |
| 4-(4′-Methoxy- | Trifluoroacetic | 2,2,2-Trifluoro-N- | 314NN | 6.48 min | 429 (M + H)⁺ |
| biphenyl-3-yl)- | anhydride | [4-(4′-methoxy- | | (t) | |
| thieno[2,3- | | biphenyl-3-yl)- | | | |
| c]pyridin-2- | | thieno[2,3- | | | |
| ylamine (A, B, E, | | c]pyridin-2-yl]- | | | |
| J) | | acetamide | | | |
| 4-(4′-Methoxy- | Methoxy- | 2-Methoxy-N-[4- | 314OO | 6.53 min | 405 (M + H)⁺ |
| biphenyl-3-yl)- | acetyl | (4′-methoxy- | | (t) | |
| thieno[2,3- | chloride | biphenyl-3-yl)- | | | |
| c]pyridin-2- | | thieno[2,3- | | | |
| ylamine (A, B, E, | | c]pyridin-2-yl]- | | | |
| J) | | acetamide | | | |
| 4- | Acetoxyacetyl | Acetic acid (4- | 314PP | 6.56 min | 413 (M − H)− |
| [2,2′]Bithiophenyl- | chloride | [2,2′]bithiophenyl- | | (t) | |
| 5-yl-thieno[2,3- | | 5-yl-thieno[2,3- | | | |
| c]pyridin-2- | | c]pyridin-2- | | | |
| ylamine (A, B, E, J, | | ylcarbamoyl)- | | | |
| Q, R) | | methyl ester | | | |
| 4- | Ethyl oxalyl | N-(4- | 314QQ | 6.99 min | 413 (M − H)− |
| [2,2′]Bithiophenyl- | chloride | [2,2′]Bithiophenyl- | | (t) | |
| 5-yl-thieno[2,3- | | 5-yl-thieno[2,3- | | | |
| c]pyridin-2- | | c]pyridin-2-yl)- | | | |
| ylamine (A, B, E, J, | | oxalamic acid ethyl | | | |
| Q, R) | | ester | | | |
| 4- | Isoxazole-5- | Isoxazole-5- | 314RR | 6.83 min | 408 (M − H)− |
| [2,2′]Bithiophenyl- | carbonyl | carboxylic acid (4- | | (t) | |
| 5-yl-thieno[2,3- | chloride | [2,2′]bithiophenyl- | | | |
| c]pyridin-2- | | 5-yl-thieno]2,3- | | | |
| ylamine (A, B, E, J, | | c]pyridin-2-yl)- | | | |
| Q, R) | | amide | | | |
| 4- | Dimethylamino- | N-(4- | 314SS | 6.00 min | 8.99(s, |
| [2,2′]Bithiophenyl- | acetyl | [2,2′]Bithiophenyl- | | (t) | 1H), 8.59(s, |
| 5-yl-thieno[2,3- | chloride | 5-yl-thieno[2,3- | | | 1H), |
| c]pyridin-2- | | c]pyridin-2-yl)-2- | | | 7.68(s, |
| ylamine (A, B, E, J, | | dimethylamino- | | | 1H), 7.56-7.57(dd, |
| Q, R) | | acetamide | | | 1H), 7.51(d, |
| | | | | | 1H), 7.45, |
| | | | | | (d, 1H), |
| | | | | | 7.41(dd, 1H), |
| | | | | | 7.14(dd, |
| | | | | | 1H), 3.24(s, 2H), |
| | | | | | 2.30(s, 6H) |
| 4′(2-Carbamoyl- | Butyl amine | 4-(4′- | 314TT | 5.48 (t) | 445 |
| thieno[2,3- | | Butylcarbamoyl- | | | (M + H)⁺ |
| c]pyridin-4- | | biphenyl-4- | | | |
| ylamino)-biphenyl- | | ylamino)- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | Methylamine | 4-(4′- | 314UU | 4.66 (t) | 403 |
| thieno[2,3- | | Methylcarbamoyl- | | | (M + H)⁺ |
| c]pyridin-4- | | biphenyl-4- | | | |
| ylamino)-biphenyl- | | ylamino)- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | Dimethylamine | 4-(4′- | 314VV | 4.88 (t) | 417 |
| thieno[2,3- | | Dimethylcarbamoyl- | | | (M + H)⁺ |
| c]pyridin-4- | | biphenyl-4- | | | |
| ylamino)-biphenyl- | | ylamino)- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | Isopropylamine | 4-(4′- | 314WW | 5.16 (t) | 431 |
| thieno[2,3- | | Isopropylcarbamoyl- | | | (M + H)⁺ |
| c]pyridin-4- | | biphenyl-4- | | | |
| ylamino)-biphenyl- | | ylamino)- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | Ethanolamine | 4-[4′-(2-Hydroxy- | 314XX | 4.41 (t) | 433 |
| thieno[2,3- | | ethylcarbamoyl)- | | | (M + H)⁺ |
| c]pyridin-4- | | biphenyl-4- | | | |
| ylamino)-biphenyl- | | ylamino]- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | 3-Amino-1- | 4-[4′-(3-Hydroxy- | 314YY | 4.50 (t) | 447 |
| thieno[2,3- | propanol | propylcarbamoyl)- | | | (M + H)⁺ |
| c]pyridin-4- | | biphenyl-4- | | | |
| ylamino)-biphenyl- | | ylamino]- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | N- | 4-[4′-(2- | 314ZZ | 7.07 (t) | 460 |
| thieno[2,3- | Ethylethylenediamine | Ethylamino- | | | (M + H)⁺ |
| c]pyridin-4- | | ethylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amide | | | |
| 4′-(2-Carbamoyl- | N-N- | 4-[4′-(2- | 314AAA | 4.39 (t) | 488 |
| thieno[2,3- | Diethyethylenediamine | Diethylamino- | | | (M + H)⁺ |
| c]pyridin-4- | | ethylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amide | | | |
| 4′-(2-Carbamoyl- | N-(2- | 4-[4′-(2-Morpholin- | 314BBB | 4.32 (t) | 502 |
| thieno[2,3- | Aminoethyl) | 4-yl- | | | (M + H)⁺ |
| c]pyridin-4- | morpholine | ethylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amide | | | |
| 4′-(2-Carbamoyl- | N-(2- | 4-[4′-(2-Piperidin- | 314CCC | 4.14 | 501 |
| thieno[2,3- | Aminoethyl)piperazine | 4-yl- | | | (M + H)⁺ |
| c]pyridin-4- | | ethylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amide | | | |
| 4′-(2-Carbamoyl- | Tert-butyl 1- | 4-[4′-(Piperazine-1- | 314DDD | 5.70 | 558 |
| thieno[2,3- | piperazinecar | carbonyl)-biphenyl- | | | (M + H)⁺ |
| c]pyridin-4- | boxylate | 4-ylamino]- | | | |
| ylamino)-biphenyl- | | thieno[2,3- | | | |
| 4-carboxylic acid | | c]pyridine-2- | | | |
| | | carboxylic acid | | | |
| | | amide | | | |
| 4′-(2-Carbamoyl- | 1- | 4-[4′-(4-Methyl- | 314EEE | 4.18 | 472 |
| thieno[2,3- | Methylpiperazine | piperazine-1- | | | (M + H)⁺ |
| c]pyridin-4- | | carbonal)-biphenyl- | | | |
| ylamino)-biphenyl- | | 4-ylamino]- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | Morpholine | 4-[4′-(Morpholine- | 314FFF | 4.86 | 459 |
| thieno[2,3- | | 4-carbonyl)- | | | (M + H)⁺ |
| c]pyridin-4- | | biphenyl-4- | | | |
| ylamino)-biphenyl- | | ylamino]- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | 2- | 4-[4′-(2-Methoxy- | 314GGG | 4.79 | 447 |
| thieno[2,3- | Methoxyethylamine | ethylcarbamoyl)- | | | (M + H)⁺ |
| c]pyridin-4- | | biphenyl-4- | | | |
| ylamino)-biphenyl- | | ylamino]- | | | |
| 4-carboxylic acid | | thieno[2,3- | | | |
| | | c]pyridine-2- | | | |
| | | carboxylic acid | | | |
| | | amide | | | |
| 4′-(2-Carbamoyl- | 1-(3- | 4-[4′-(3-Pyrrolidin- | 314HHH | 4.30 | 450 |
| thieno[2,3- | Aminopropyl) | 1-yl- | | | (M + H)⁺ |
| c]pyridin-4- | pyrrolidine | propylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amid | | | |
| 4′-(2-Carbamoyl- | 1-(2- | 4-[4′-(2-Pyrrolidin- | 314III | 4.31 (t) | 486 |
| thieno[2,3- | Aminoethyl)pyrrolidine) | 1-yl- | | | (M + H)⁺ |
| c]pyridin-4- | | ethylcarbamoyl)- | | | |
| ylamino)-biphenyl- | | biphenyl-4- | | | |
| 4-carboxylic acid | | ylamino]- | | | |
| | | thieno[2,3- | | | |
| | | c]pyridine-2- | | | |
| | | carboxylic acid | | | |
| | | amide | | | |

Other compounds obtained using general procedure Q are shown (Table 13).

TABLE 13
Examples synthesized using general procedure Q

| | | | | HPLC Rₜ | |
|---|---|---|---|---|---|
| Acid precursor | Reagent | Product | Ex # | (Method) | m/z |
| 4-(4-tert-Butyl- | NA | [4-(4-tert-Butyl- | 315 | 9.96 min | 398 (M + H)⁺ |
| phenylamino)- | | phenylamino)- | | (b) | |
| thieno[2,3- | | thieno[2,3-c]pyridin- | | | |
| c]pyridine-2- | | 2-yl]-carbamic acid | | | |
| carboxylic acid | | tert-butyl ester | | | |
| (A, B, I, E) | | | | | |
| 4-(3-Chloro-phenyl)- | NA | [4-(3-Chloro- | 316 | 9.83 min | , 362.9 (M + H)⁺ |
| thieno[2,3- | | phenyl)-thieno[2,3- | | (b) | (i) |
| c]pyridine-2- | | c]pyridin-2-yl]- | | | |
| carboxylic acid | | carbamic acid tert- | | | |
| (A, B, J, E) | | butyl ester | | | |
| 4-(4′-Chloro- | NA | [4-(4′-Chloro- | 317 | 11.05 min | 451, 453 (M − H)⁻ |
| biphenyl-4-yloxy)- | | biphenyl-4-yloxy)- | | (b) | |
| thieno[2,3- | | thieno[2,3-c]pyridin- | | | |
| c]pyridine-2- | | 2-yl]-carbamic acid | | | |
| carboxylic acid | | tert-butyl ester | | | |
| (A, D, E, J) | | | | | |
| 4-[2,2′]Bithiophenyl- | NA | (4- | Q1 | 7.7 min | 413.0 (M − H)⁻ |
| 5-yl-thieno[2,3- | | [2,2′]Bithiophenyl-5- | | (t) | |
| c]pyridine-2- | | yl-thieno[2,3- | | | |
| carboxylic acid (A, | | c]pyridin-2-yl)- | | | |
| B, E, J) | | carbamic acid tert- | | | |
| | | butyl ester | | | |

Other compounds obtained using general procedure R are shown (Table 14).

TABLE 14
Examples synthesized using general procedure R

| Boc-protected amine | | | | HPLC Rₜ | |
|---|---|---|---|---|---|
| precursor | Reagent | Product | Ex # | (Method) | m/z |
| N*4*-(4-tert-Butyl- | Trifluoroacetic | N-[4-(4-tert-Butyl- | 318 | 8.59 | 392.2 |
| phenyl)-thieno[2,3- | anhydride | phenylamino)-thieno[2,3- | | min (b) | (M − H)⁻ |
| c]pyridine-2,4- | | c]pyridin-2-yl]-2,2,2- | | | (i) |
| diamine | | trifluoro-acetamide | | | |
| (A, B, I, E, Q, R) | | | | | |
| [4-(4′-Chloro- | Trifluoroacetic | 4-(4′-Chloro-biphenyl-4- | 319 | 9.22 | 353, 355 |
| biphenyl-4-yloxy)- | anhydride | yloxy)-thieno[2,3- | | min (b) | (M + H)⁺ |
| thieno[2,3-c]pyridin- | | c]pyridin-2-ylamine | | | |
| 2-yl]-carbamic acid | | | | | |
| tert-butyl ester | | | | | |
| (A, D, E, J, Q) | | | | | |
| [4-(4′-Chloro- | Acetic | N-[4-(4′-Chloro-biphenyl- | 320 | 9.31 | 395, 397 |
| biphenyl-4-yloxy)- | anhydride | 4-yloxy)-thieno[2,3- | | min (b) | (M + H)⁺ |
| thieno[2,3-c]pyridin- | | c]pyridin-2-yl]-acetamide | | | |
| 2-yl]-carbamic acid | | | | | |
| tert-butyl ester | | | | | |
| (A, D, E, J, Q) | | | | | |
| [4-(4′-Chloro- | Isocyanato- | {3-[4-(4′-Chloro-biphenyl- | 321 | 9.32 | 482, 484 |
| biphenyl-4-yloxy)- | acetic | 4-yloxy)-thieno[2,3- | | min (b) | (M + H)⁺ |
| thieno[2,3-c]pyridin- | acid ethyl | c]pyridin-2-yl]-ureido}- | | | |
| 2-yl]-carbamic acid | ester | acetic acid ethyl ester | | | |
| tert-butyl ester | | | | | |
| (A, D, E, J, Q) | | | | | |
| 4-(4-Bromo- | NA | 4-(4-Bromo- | 322 | 1.91 | 408/410 |
| phenylamino)- | | phenylamino)-thieno[2,3- | | min (i) | (M + H)⁺ |
| thieno[2,3- | | c]pyridine-2-carboxylic | | | |
| c]pyridine-2- | | acid (2-hydroxy-ethoxy)- | | | |
| carboxylic acid (2- | | amide | | | |
| tert-butoxy-ethoxy)- | | | | | |
| amide (A, C, I) | | | | | |
| 4-[4-(2-Carbamoyl- | N/A | 4-{4-[(Piperidine-4- | 323 | 6.25 | 381 |
| thieno[2,3-c]pyridin- | | carbonyl)-amino]- | | min (a) | (M + H)⁺ |
| 4-yl)- | | phenyl}-thieno[2,3- | | | |
| phenylcarbamoyl]- | | c]pyridine-2- | | | |
| piperidine-1- | | carboxylic acid amide | | | |
| carboxylic acid tert- | | | | | |
| butyl ester (A, C, J, | | | | | |
| O) | | | | | |
| 7-Amino-4-(3′- | N/A | 7-Amino-4-(3′- | 323 | 5.17 | 412.9 |
| trifluoromethyl- | | trifluoromethyl- | A | min (q) | (M − H)⁻ |
| biphenyl-3-yl)- | | biphenyl-3-yl)- | | | |
| thieno[2,3- | | thieno[2,3- | | | |
| c]pyridine-2- | | c]pyridine-2- | | | |
| carboxylic acid tert- | | carboxylic acid | | | |
| butyl ester | | trifluoroacetate salt | | | |
| (A,B,OO,AAA,J,I,II) | | | | | |
| 7-Amino-4-(4′- | N/A | 7-Amino-4-(4′-cyano- | 323 | 4.68 | 369.7 |
| cyano-biphenyl-3- | | biphenyl-3-yl)- | B | min (q) | (M − H)⁻ |
| yl)-thieno[2,3- | | thieno[2,3-c]pyridine-2- | | | |
| c]pyridine-2- | | carboxylic acid acetate | | | |
| carboxylic acid tert- | | salt | | | |
| butyl ester | | | | | |
| (A,B,OO,AAA,II(J,I | | | | | |
| II),I) | | | | | |

Other compounds obtained using general procedure S are shown (Table 15).

TABLE 15
Examples synthesized using general procedure S

| Carboxylic Acid | | | | HPLC Rₜ | |
|---|---|---|---|---|---|
| Precursor | Amine | Product | Ex # | (Method) | m/z |
| 4-(2-Carbamoyl- | Aniline | 4-(4-Phenylacetyl- | 324 | 9.06 min | 374 |
| thieno[2,3-c]pyridin- | | phenyl)-thieno[2,3- | | (a) | |
| 4-yl)-benzoic acid | | c]pyridine-2- | | | |
| (A, C, J) | | carboxylic acid amide | | | |
| 4-(4-Amino-phenyl)- | N- | 4-[4-(2-Phenylamino- | 325 | 8.96 min | 403 |
| thieno[2,3- | Phenylglycine | acetylamino)-phenyl]- | | (a) | |
| c]pyridine-2- | | thieno[2,3-c]pyridine- | | | |
| carboxylic acid | | 2-carboxylic acid | | | |
| amide | | amide | | | |
| 4-(3-Amino- | BOC- | 4-[3-(2-Carbamoyl- | 326 | 9.43 min | 481 |
| phenyl)thieno[2,3- | piperidine-4- | thieno[2,3-c]pyridin- | | (a) | (M + H)⁺ |
| c]pyridine-2- | carboxylic acid | 4-yl)- | | | |
| carboxylic acid | | phenylcarbamoyl]- | | | |
| amide | | piperidine-1- | | | |
| (A, C, J) | | carboxylic acid tert- | | | |
| | | butyl ester | | | |
| 4-(3-Amino- | Isonicotinic | 4-{3-[(Pyridine-4- | 327 | 7.55 min | 375 |
| phenyl)thieno[2,3- | Acid | carbonyl)-amino]- | | (a) | (M + H)⁺ |
| c]pyridine-2- | | phenyl}-thieno[2,3- | | | |
| carboxylic acid | | c]pyridine-2- | | | |
| amide | | carboxylic acid amide | | | |
| (A, C, J) | | | | | |
| 4-(3-Amino- | 1- | 4-{3-[(1-Methyl- | 328 | 8.44 min | 352 |
| phenyl)thieno[2,3- | Methylcyclopropane | cyclopropanecarbonyl)- | | (a) | (M + H)⁺ |
| c]pyridine-2- | carboxylic | amino]-phenyl}- | | | |
| carboxylic acid | acid | thieno[2,3-c]pyridine- | | | |
| amide | | 2-carboxylic acid | | | |
| (A, C, J) | | amide | | | |
| 4-(4-Amino- | BOC- | 4-[4-(2-Carbamoyl- | 329 | 9.29 min | 481 |
| phenyl)thieno[2,3- | piperidine-4- | thieno[2,3-c]pyridin- | | (a) | (M + H)⁺ |
| c]pyridine-2- | carboxylic acid | 4-yl)- | | | |
| carboxylic acid | | phenylcarbamoyl]- | | | |
| amide | | piperidine-1- | | | |
| (A, C, J) | | carboxylic acid tert- | | | |
| | | butyl ester | | | |
| 4-(2-Carbamoyl- | Isopropyl | 4-(4- | 330 | 7.86 min | 340 |
| thieno[2,3-c]pyridin- | amine | Isopropylcarbamoyl- | | (a) | (M + H)⁺ |
| 4-yl)-benzoic acid | | phenyl)-thieno[2,3- | | | |
| (A, C, J, sp ex XX) | | c]pyridine-2- | | | |
| | | carboxylic acid amide | | | |
| 4-(2-Carbamoyl- | 1- | 4-[4-(2-Piperidin-1-yl- | 331 | 6.15 min | 381 |
| thieno[2,3-c]pyridin- | Methylpiperazine | acetylamino)-phenyl]- | | (a) | (M + H)⁺ |
| 4-yl)-benzoic acid | | thieno[2,3-c]pyridine- | | | |
| (A, C, J, sp ex XX) | | 2-carboxylic acid | | | |
| | | amide | | | |
| 4-(Biphenyl-4- | O-Benzyl- | 4-(Biphenyl-4-yloxy)- | 332 | 12.28 min | 453 (M + H)⁺ |
| yloxy)-thieno[2,3- | hydroxylamine | thieno[2,3-c]pyridine- | | (a) | |
| c]pyridine- | hydrochloride | 2-carboxylic | | | |
| 2-carboxylic | | acid benzyloxy- | | | |
| acid (A, B, D, E, J) | | amide | | | |
| 4-(Biphenyl-4- | 2-Methoxy- | 4-(Biphenyl-4- | 333 | 10.46 min | 404 (M + H)⁺ |
| ylamino)-thieno[2,3-c | ethylamine | ylamino)-thieno[2,3- | | (a) | |
| ]pyridine-2- | | c]pyridine- | | | |
| carboxylic acid | | 2-carboxylic | | | |
| ditrifluoroacetate | | acid (2-hydroxy- | | | |
| (A, B, I, X) | | ethyl)-amide | | | |
| 4-(Biphenyl-4- | Piperidine | [4-(Biphenyl-4- | 334 | 11.77 min | 414 (M + H)⁺ |
| ylamino)-thieno[2,3-c | | ylamino)-thieno[2,3- | | (a) | |
| ]pyridine-2- | | c]pyridin-2-yl]- | | | |
| carboxylic acid | | piperidin-1-yl-methanone | | | |
| ditrifluoroacetate (A, | | | | | |
| B, I, X) | | | | | |
| 4-(Biphenyl-4- | tert-Butyl-1- | 4-[4-(Biphenyl-4- | 335 | 12.08 min | 515 (M + H)⁺ |
| ylamino)-thieno[2,3-c | piperazine | ylamino)-thieno[2, | | (a) | |
| ]pyridine-2- | carboxylate | 3-c]pyridine-2- | | | |
| carboxylic acid | | carbonyl]-piperazine- | | | |
| ditrifluoroacetate (A, | | 1-carboxylic acid tert- | | | |
| B, I, X) | | butyl ester | | | |
| 4-(Biphenyl-4- | 1-Methyl- | [4-(Biphenyl-4- | 336 | 9.45 min | 429 (M + H)⁺ |
| ylamino)-thieno[2,3-c | piperizine | ylamino)-thieno[2,3- | | (a) | |
| ]pyridine-2- | | c]pyridin-2-yl]-(4- | | | |
| carboxylic acid | | methyl-piperazin- | | | |
| ditrifluoroacetate (A, | | 1-yl)-methanone | | | |
| B, I, X) | | | | | |
| 4-(Biphenyl-4- | Morpholine | [4-(Biphenyl-4- | 337 | 10.49 min | 414 (M − H)⁻ |
| ylamino)-thieno[2,3-c | | ylamino)-thieno[2,3- | | (a) | |
| ]pyridine-2- | | c]pyridin-2-yl]- | | | |
| carboxylic acid | | morpholin-4-yl-methanone | | | |
| ditrifluoroacetate (A, | | | | | |
| B, I, X) | | | | | |
| 4-(Biphenyl-4- | 4- | 4-(Biphenyl-4- | 338 | 11.17 min | 421 (M − H)⁻ |
| ylamino)-thieno[2,3-c | Aminopyridine | ylamino)-thieno[2,3-c | | (a) | |
| ]pyridine-2- | | ]pyridine-2-carboxylic | | | |
| carboxylic acid | | acid pyridin- | | | |
| ditrifluoroacetate (A, | | 4-ylamide | | | |
| B, I, X) | | | | | |
| 4-(Biphenyl-4- | 4- | 4-(Biphenyl-4- | 339 | 10.98 min | 424 (M + H)⁺ |
| ylamino)-thieno[2,3-c | Aminopyrimidine | ylamino)-thieno[2,3-c | | (a) | |
| ]pyridine-2- | | ]pyridine-2-carboxylic | | | |
| carboxylic acid | | acid pyrimidin- | | | |
| ditrifluoroacetate (A, | | 4-ylamide acetate | | | |
| B, I, X) | | salt | | | |
| 4-(Biphenyl-4- | N-(3- | 4-(Biphenyl-4- | 340 | 9.87 min | 473 (M + H)⁺ |
| ylamino)-thieno[2,3-c | Aminopropyl) | ylamino)-thieno[2,3-c | | (a) | |
| ]pyridine-2- | morpholine | ]pyridine-2-carboxylic | | | |
| carboxylic acid | | acid (3-morpholin- | | | |
| ditrifluoroacetate (A, | | 4-yl-propyl)- | | | |
| B, I, X) | | amide diacetate salt | | | |
| 4-(Biphenyl-4- | Ethanolamine | 4-(Biphenyl-4- | 341 | 9.39 min | 390 (M + H)⁺ |
| ylamino)-thieno[2,3-c | | ylamino)-thieno[2,3-c | | (a) | |
| ]pyridine-2- | | ]pyridine-2-carboxylic | | | |
| carboxylic acid | | acid (2-hydroxy- | | | |
| ditrifluoroacetate (A, | | ethyl)-amide | | | |
| B, I, X) | | | | | |
| 4-(Biphenyl-4- | Propylamine | 4-(Biphenyl-4- | 342 | 11.41 min | 388 (M + H)⁺ |
| ylamino)-thieno[2,3-c | | ylamino)-thieno[2,3-c | | (a) | |
| ]pyridine-2- | | ]pyridine-2-carboxylic | | | |
| carboxylic acid | | acid propylamide | | | |
| ditrifluoroacetate (A, | | | | | |
| B, I, X) | | | | | |
| 4-(Biphenyl-4- | Piperazinone | 4-[4-(Biphenyl-4- | 343 | 9.17 min | 429 (M + H)⁺ |
| ylamino)-thieno[2,3-c | | ylamino)-thieno[2, | | (a) | |
| ]pyridine-2- | | 3-c]pyridine-2- | | | |
| carboxylic acid | | carbonyl]-piperazin- | | | |
| trifluoroacetate (A, | | 2-one | | | |
| B, I, X) | | | | | |
| 4-(Biphenyl-4- | Ethylenediamine | 4-(Biphenyl-4- | 344 | 9.15 min | 389 (M + H)⁺ |
| ylamino)-thieno[2,3-c | | ylamino)-thieno[2,3-c | | (a) | |
| ]pyridine-2- | | ]pyridine-2-carboxylic | | | |
| carboxylic acid | | acid (2-amino- | | | |
| trifluoroacetate | | ethyl)-amide | | | |
| (A, B, I, X) | | | | | |
| 4-(Biphenyl-4- | N,N-Dimtheylethylenediamine | 4-(Biphenyl-4- | 345 | 9.89 min | 417 (M + H)⁺ |
| ylamino)-thieno[2,3-c | | ylamino)-thieno[2,3-c | | (a) | |
| ]pyridine-2- | | ]pyridine-2-carboxylic | | | |
| carboxylic acid | | acid (2-dimethylamino- | | | |
| trifluoroacetate | | ethyl)- | | | |
| (A, B, I, X) | | amide | | | |
| 4-(Biphenyl-4- | β-Alanine tert- | 3-{[4-(Biphenyl-4- | 346 | 11.94 min | 474 (M + H)⁺ |
| ylamino)-thieno[2,3-c | butyl ester | ylamino)-thieno[2, | | (a) | |
| ]pyridine-2- | hydrochloride | 3-c]pyridine-2- | | | |
| carboxylic acid | | carbonyl]-amino}-propionic | | | |
| trifluoroacetate | | acid tert-butyl | | | |
| (A, B, I, X) | | ester | | | |
| 4-(Biphenyl-4- | O- | 4-(Biphenyl-4- | 347 | 10.84 min | 402 (M + H)⁺ |
| ylamino)-thieno[2,3-c | Allylhydroxylamine | ylamino)-thieno[2,3-c | | (a) | |
| ]pyridine-2- | hydrochloride | ]pyridine-2-carboxylic | | | |
| carboxylic acid | | acid allyloxy- | | | |
| trifluoroacetate | | amide | | | |
| (A, B, I, X) | | | | | |
| 4-(Biphenyl-4- | Methyl | 4-(Biphenyl-4- | 348 | 10.08 min | 375 (M + H)⁺ |
| ylamino)-thieno[2,3-c | hydrazine | ylamino)-thieno[2,3-c | | (a) | |
| ]pyridine-2- | | ]pyridine-2-carboxylic | | | |
| carboxylic acid | | acid N′-methyl- | | | |
| trifluoroacetate | | hydrazide | | | |
| (A, B, I, X) | | | | | |
| 4-(Biphenyl-4- | O-iso- | 4-(Biphenyl-4- | 349 | 11.73 min | 418 (M + H)⁺ |
| ylamino)-thieno[2,3-c | butylhydroxylamine | ylamino)-thieno[2,3-c | | (a) | |
| ]pyridine-2- | hydrochloride | ]pyridine-2-carboxylic | | | |
| carboxylic acid | | acid isobutoxy- | | | |
| trifluoroacetate | | amide | | | |
| (A, B, I, X) | | | | | |
| 4-(Biphenyl-4- | O-tert-butyl-L- | (S)-2-{[4-(Biphenyl-4- | 350 | 13.68 min | 546 (M + H)⁺ |
| ylamino)-thieno[2,3-c | serine tert-butyl | ylamino)-thieno | | (a) | |
| ]pyridine-2- | ester | [2,3-c]pyridine-2- | | | |
| carboxylic acid | hydrochloride | carbonyl]-amino | | | |
| trifluoroacetate | | }-3-tert-butoxy- | | | |
| (A, B, I, X) | | propionic acid tert- | | | |
| | | butyl ester | | | |
| 4-(Biphenyl-4- | O-(5-Chloro- | 4-(Biphenyl-4- | 351 | 11.61 min | 494 (M + H)⁺ |
| ylamino)-thieno[2,3-c | [1,2,3]thiadiazol- | ylamino)-thieno[2,3-c | | (o) | |
| ]pyridine-2- | 4-ylmethyl)- | ]pyridine-2-carboxylic | | | |
| carboxylic acid | hydroxylamine | acid (5-chloro- | | | |
| trifluoroacetate | hydrochloride | [1,2,3]thiadiazol-4- | | | |
| (A, B, I, X) | | ylmethoxy)-amide | | | |
| 4-(Biphenyl-4- | 1- | 4-(Biphenyl-4- | 352 | 12.76 min | 466 (M + H)⁺ |
| ylamino)-thieno[2,3-c | [(Ammoniooxy) | ylamino)-thieno[2,3-c | | (o) | |
| ]pyridine-2- | methyl]-4- | ]pyridine-2-carboxylic | | | |
| carboxylic acid | methylbenzene | acid (4-methyl- | | | |
| trifluoroacetate | chloride | benzyloxy)-amide | | | |
| (A, B, I, X) | | | | | |
| 4-(Biphenyl-4- | 1- | 4-(Biphenyl-4- | 353 | 12.78 min | 486 (M + H)⁺ |
| ylamino)-thieno[2,3-c | [(Ammoniooxy) | ylamino)-thieno[2,3-c | | (o) | |
| ]pyridine-2- | methyl]-2- | ]pyridine-2-carboxylic | | | |
| carboxylic acid | chlorobenzene | acid (2-chloro- | | | |
| trifluoroacetate | chloride | benzyloxy)-amide | | | |
| (A, B, I, X) | | | | | |
| 4-(Biphenyl-4- | 1- | 4-(Biphenyl-4- | 354 | 12.90 min | 486 (M + H)⁺ |
| ylamino)-thieno[2,3-c | [(Ammoniooxy) | ylamino)-thieno[2,3-c | | (o) | |
| ]pyridine-2- | methyl]-3- | ]pyridine-2-carboxylic | | | |
| carboxylic acid | chlorobenzene | acid (3-chloro- | | | |
| trifluoroacetate | chloride | benzyloxy)-amide | | | |
| (A, B, I, X) | | | | | |
| 4-(Biphenyl-4- | 1- | 4-(Biphenyl-4- | 355 | 12.32 min | 482 (M + H)⁺ |
| ylamino)-thieno[2,3- | [(Ammoniooxy) | ylamino)-thieno[2,3-c | | (o) | |
| ]pyridine-2- | methyl]-2- | ]pyridine-2-carboxylic | | | |
| carboxylic acid | methoxybenzene | acid (2-methoxy- | | | |
| trifluoroacetate | chloride | benzyloxy)-amide | | | |
| (A, B, I, X) | | | | | |
| 4-(Biphenyl-4- | 1- | 4-(Biphenyl-4- | 356 | 12.21 min | 482 (M + H)⁺ |
| ylamino)-thieno[2,3-c | [(Ammoniooxy) | ylamino)-thieno[2,3-c | | (o) | |
| ]pyridine-2- | methyl]-3- | ]pyridine-2-carboxylic | | | |
| carboxylic acid | methoxybenzene | acid (3-methoxy- | | | |
| trifluoroacetate | chloride | benzyloxy)-amide | | | |
| (A, B, I, X) | | | | | |
| 4-(4-Bromo- | O-Methyl- | 4-(4-Bromo- | 357 | 2.80 min | 378/380 |
| phenylamino)- | hydroxylamine | phenylamino)- | | (i) | (M H)⁺ |
| thieno[2,3- | hydrochloride | thieno[2,3-c]pyridine- | | | |
| c]pyridine-2- | | 2-carboxylic acid | | | |
| carboxylic acid | | methoxy-amide | | | |
| (A, B, I, E) | | | | | |
| 4-(4-Bromo- | O-(2-tert- | 4-(4-Bromo- | 358 | 2.86 min | 464/466 |
| phenylamino)- | Butoxy-ethyl)- | phenylamino)- | | (i) | (M H)⁺ |
| thieno[2,3- | hydroxylamine | thieno[2,3-c]pyridine- | | | |
| c]pyridine-2- | | 2-carboxylic acid (2- | | | |
| carboxylic acid | | tert-butoxy-ethoxy)- | | | |
| (A, B, I, E) | | amide | | | |
| 4-(Biphenyl-4- | 1- | 4-(Biphenyl-4- | 359 | 12.14 min | 482 (M + H)⁺ |
| ylamino)-thieno[2,3-c | [(Ammoniooxy) | ylamino)-thieno[2,3-c | | (o) | |
| ]pyridine-2- | methyl]-4- | ]pyridine-2-carboxylic | | | |
| carboxylic acid | methoxybenzene | acid (4-methoxy- | | | |
| trifluoroacetate | chloride | benzyloxy)-amide | | | |
| (A, B, I, X) | | | | | |

Other compounds obtained using general procedure T are shown (Table 16).

TABLE 16
Examples synthesized using general procedure T

| Thienopyridine | | | | HPLC Rₜ | |
|---|---|---|---|---|---|
| precursor | Phenol precursor | Product | Ex # | (Method) | m/z |
| 4-Bromo-2- | Biphenyl-2-ol | 4-(Biphenyl-3- | 360 | 1.78 min (i) | 371.9 (M + H)⁺ |
| (1H-tetrazol-5- | | yloxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | Biphenyl-2-ol | 4-(Biphenyl-2- | 361 | 1.67 min (i) | 371.9 (M + H)⁺ |
| (1H-tetrazol-5- | | yloxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | 3-Phenoxy-phenol | 4-(3-Phenoxy- | 362 | 1.77 min (i) | 386.0 (M − H)− |
| (1H-tetrazol-5- | | phenoxy)-2-(1H- | | | |
| yl)-thieno | | tetrazo | | | |
| [2,3-c]pyridine | | 1-5-yl)-thieno[2,3- | | | |
| (A, C, F, G) | | c]pyridine | | | |
| 4-Bromo-2- | 3-tert-Butyl-phenol | 4-(3-tert-Butyl- | 363 | 1.86 min (i) | 350.1 (M − H)− |
| (1H-tetrazol-5- | | phenoxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | 2-tert-Butyl-phenol | 4-(2-tert-Butyl- | 364 | 1.76 min (i) | 350.1 (M − H)− |
| (1H-tetrazol-5- | | phenoxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | 4-tert-Butyl-phenol | 4-(4-tert-Butyl- | 365 | 2.02 min (i) | 350.1 (M − H)− |
| (1H-tetrazol-5- | | phenoxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | Dibenzofuran-2-ol | 4-(Dibenzofuran-2- | 366 | 1.82 min (i) | 385.9 (M + H)+ |
| (1H-tetrazol-5- | | yloxy)-2-(1H-tetrazol- | | | |
| yl)-thieno | | 5-yl)-thieno[2,3- | | | |
| [2,3-c]pyridine | | c]pyridine | | | |
| (A, C, F, G) | | | | | |
| 4-Bromo-2- | 2-Hydroxy-fluoren- | 2-[2-(1H-Tetrazol-5- | 367 | 1.73 min (i) | 396.1 (M − H)− |
| (1H-tetrazol-5- | 9-one | yl)-thieno[2,3- | | | |
| yl)-thieno | | c]pyridin-4-yloxy]- | | | |
| [2,3-c]pyridine | | fluoren-9-one | | | |
| (A, C, F, G) | | | | | |
| 4-Bromo-2- | 3-Morpholin-4-yl- | 4-(3-Morpholin-4-yl- | 368 | 0.95 min (i) | 379.0 (M − H)− |
| (1H-tetrazol-5- | phenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo- | 4-Cyclopentylphenol | 4-(4-Cyclopentyl- | 369 | 2.46 min (i) | 330.2 (M + H)⁺ |
| thieno[2,3- | | phenoxy)-thieno[2, | | | |
| c]pyridine-2- | | 3-c]pyridine-2- | | | |
| carbonitrile | | carboxylic acid amide | | | |
| (A, C, | | | | | |
| F) | | | | | |
| 4-Bromo-2- | 4-Cyclopentylphenol | 4-(4-Cyclopentyl- | 370 | 2.01 min (i) | 364.1 (M + H)⁺ |
| (1H-tetrazol-5- | | phenoxy)-2-(1H- | | | |
| yl)-thieno | | tetrazol-5-yl)- | | | |
| [2,3-c]pyridine | | thieno[2,3c]pyridine | | | |
| (A, C, F, G) | | | | | |
| 4-Bromo-2- | 4-Phenoxy-phenol | 4-(4-Phenoxy- | 371 | 1.88 min (i) | 388.1 (M + H)⁺ |
| (1H-tetrazol-5- | | phenoxy)-2-(1H- | | | |
| yl)-thieno | | tetrazol- | | | |
| [2,3-c]pyridine | | 5-yl)-thieno[2,3- | | | |
| (A, C, F, G) | | c]pyridine | | | |
| 4-Bromo-2- | 2-Isopropoxyphenol | 4-(2-Isopropoxy- | 372 | 1.76 min (l) | 352.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2- | 4-(2-Cyclopentyl- | 373 | 1.99 min (l) | 362.4 (M + H)⁻ |
| (1H-tetrazol-5- | Cyclopentylphenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 5-Isopropyl-3- | 4-(3-Isopropyl-5- | 374 | 1.97 min (l) | 350.4 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 2-Tert-butyl-4- | 4-(2-tert-Butyl-4- | 375 | 2.12 min (l) | 378.5 (M + H)⁻ |
| (1H-tetrazol-5- | ethylphenol | ethyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 4-Cumylphenol | 4-[4-(1-Methyl-1- | 376 | 2.12 min (l) | 412.5 (M + H)⁻ |
| (1H-tetrazol-5- | | phenyl-ethyl)- | | | |
| yl)-thieno | | phenoxy]-2-(1H- | | | |
| [2,3-c]pyridine | | tetrazol-5-yl)- | | | |
| (A, C, F, G) | | thieno[2,3-c]pyridine | | | |
| 4-Bromo-2- | 3,5-Di-tert- | 4-(3,5-Di-tert-butyl- | 377 | 2.26 min (l) | 406.5 (M + H)⁻ |
| (1H-tetrazol-5- | butylphenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 5- | 5-[2-(1H-Tetrazol-5- | 378 | 1.54 min (l) | 345.4 (M + H)⁻ |
| (1H-tetrazol-5- | Hydroxyisoquinoline | yl)-thieno[2,3- | | | |
| yl)-thieno | | c]pyridin-4-yloxy]- | | | |
| [2,3-c]pyridine | | isoquinoline | | | |
| (A, C, F, G) | | | | | |
| 4-Bromo-2- | 5,6,7,8-Tetrahydro- | 4-(5,6,7,8- | 379 | 1.91 min (l) | 348.4 (M + H)⁻ |
| (1H-tetrazol-5- | 1-naphthol | Tetrahydro- | | | |
| yl)-thieno | | naphthalen-1-yloxy)- | | | |
| [2,3-c]pyridine | | 2-(1H-tetrazol-5-yl)- | | | |
| (A, C, F, G) | | thieno[2,3-c]pyridine | | | |
| 4-Bromo-2- | 4-Chloro-3-ethyl-5- | 4-(4-Chloro-3-ethyl- | 380 | 2.02 min (l) | 370.9 (M + H)⁻ |
| (1H-tetrazol-5- | methylphenol | 5-methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 4-(1-Indanyl)phenol | 4-(4-Indan-1-yl- | 381 | 2.13 min (l) | 410.5 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3,4-Dimethyl-6- | 4-(2-Ethyl-4,5- | 382 | 1.98 min (l) | 350.4 (M + H)⁻ |
| (1H-tetrazol-5- | ethylphenol | dimethyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 3-Ethyl-5- | 4-(3-Ethyl-5-methyl- | 383 | 1.89 min (l) | 336.4 (M + H)⁻ |
| (1H-tetrazol-5- | methylphenol | phenoxy)-2-(1H- | | | |
| yl)-thieno | | tetrazol-5-yl) | | | |
| [2,3-c]pyridine | | thieno[2,3-c]pyridine | | | |
| (A, C, F, G) | | | | | |
| 4-Bromo-2- | 2-Benzyl-4- | 4-(2-Benzyl-4- | 384 | 2.03 min (l) | 398.5 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 4-Methylthio-ortho- | 4-(2-Methyl-4- | 385 | 1.84 min (l) | 354.4 (M + H)⁻ |
| (1H-tetrazol-5- | cresol | methylsulfanyl- | | | |
| yl)-thieno | | phenoxy)-2-(1H- | | | |
| [2,3-c]pyridine | | tetrazol-5-yl)- | | | |
| (A, C, F, G) | | thieno[2,3-c]pyridine | | | |
| 4-Bromo-2- | 4-Tert-butyl-2- | 4-(5-tert-Butyl- | 386 | 2.18 min (l) | 426.5 (M + H)⁻ |
| (1H-tetrazol-5- | phenylphenol | biphenyl-2-yloxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 3- | {3-[2-(1H-Tetrazol-5- | 387 | 1.73 min (l) | 366.4 (M + H)⁻ |
| (1H-tetrazol-5- | Hydroxyphenylacetic | yl)-thieno[2,3- | | | |
| yl)-thieno | acid methyl ester | c]pyridin-4-yloxy]- | | | |
| [2,3-c]pyridine | | phenyl}-acetic acid | | | |
| (A, C, F, G) | | methyl ester | | | |
| 4-Bromo-2- | 2-Isopropylphenol | 4-(2-Isopropyl- | 388 | 1.86 min (l) | 336.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2-Sec-butylphenol | 4-(2-sec-Butyl- | 389 | 1.94 min (l) | 350.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2,3-Dimethylphenol | 4-(2,3-Dimethyl- | 390 | 1.80 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2,4-Dimethylphenol | 4-(2,4-Dimethyl- | 391 | 1.81 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2,5-Dimethylphenol | 4-(2,5-Dimethyl- | 392 | 1.80 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2- | 4-(2-Benzyl- | 393 | 1.93 min (l) | 384.5 (M + H)⁻ |
| (1H-tetrazol-5- | Hydroxydiphenylmethane | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2-Ethylphenol | 4-(2-Ethyl-phenoxy)- | 394 | 1.79 min (l) | 322.4 (M + H)⁻ |
| (1H-tetrazol-5- | | 2-(1H-tetrazol-5-yl)- | | | |
| yl)-thieno | | thieno[2,3-c]pyridine | | | |
| [2,3-c]pyridine | | | | | |
| (A, C, F, G) | | | | | |
| 4-Bromo-2- | 3-Cyanophenol | 3-[2-(1H-Tetrazol-5- | 395 | 1.59 min (l) | 319.3 (M + H)⁻ |
| (1H-tetrazol-5- | | yl)-thieno[2,3- | | | |
| yl)-thieno | | c]pyridin-4-yloxy]- | | | |
| [2,3-c]pyridine | | benzonitrile | | | |
| (A, C, F, G) | | | | | |
| 4-Bromo-2- | 3-Chlorophenol | 4-(3-Chloro- | 396 | 1.75 min (l) | 328.8 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3-Methoxyphenol | 4-(3-Methoxy- | 397 | 1.66 min (l) | 324.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3-Isopropylphenol | 4-(3-Isopropyl- | 398 | 1.93 min (l) | 336.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3,4-Dimethylphenol | 4-(3,4-Dimethyl- | 399 | 1.82 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3,5-Dimethylphenol | 4-(3,5-Dimethyl- | 400 | 1.84 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 4-Chloro-2- | 4-(4-Chloro-2- | 401 | 1.86 min (l) | 342.8 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 2,3- | 4-(2,3-Dimethoxy- | 402 | 1.59 min (l) | 354.4 (M + H)⁻ |
| (1H-tetrazol-5- | Dimethoxyphenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3,5- | 4-(3,5-Dimethoxy- | 403 | 1.70 min (l) | 354.4 (M + H)⁻ |
| (1H-tetrazol-5- | Dimethoxyphenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3,4- | 4-(3,4-Dimethoxy- | 404 | 1.54 min (l) | 354.4 (M + H)⁻ |
| (1H-tetrazol-5- | Dimethoxyphenol | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 4-Tert-butyl-2- | 4-(4-tert-Butyl-2- | 405 | 2.09 min (l) | 364.5 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 3-Benzyloxyphenol | 4-(3-Benzyloxy- | 406 | 1.98 min (l) | 400.5 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2-Ethoxy-4- | 4-(2-Ethoxy-4- | 407 | 1.80 min (l) | 352.4 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | Thymol | 4-(2-Isopropyl-5- | 408 | 1.99 min (l) | 350.4 (M + H)⁻ |
| (1H-tetrazol-5- | | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | 1-Naphthol | 4-(Naphthalen-1- | 409 | 1.86 min (l) | 344.4 (M + H)⁻ |
| (1H-tetrazol-5- | | yloxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3-Ethoxyphenol | 4-(3-Ethoxy- | 410 | 1.76 min (l) | 338.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 2-Methoxy-5- | 4-(2-Methoxy-5- | 411 | 1.71 min (l) | 338.4 (M + H)⁻ |
| (1H-tetrazol-5- | methylphenol | methyl-phenoxy)-2- | | | |
| yl)-thieno | | (1H-tetrazol-5-yl)- | | | |
| [2,3-c]pyridine | | thieno[2,3-c]pyridine | | | |
| (A, C, F, G) | | | | | |
| 4-Bromo-2- | Methyl 3- | 3-[2-(1H-Tetrazol-5- | 412 | 1.40 min (l) | 352.4 (M + H)⁻ |
| (1H-tetrazol-5- | hydroxybenzoate | yl)-thieno[2,3- | | | |
| yl)-thieno | | c]pyridin-4-yloxy]- | | | |
| [2,3-c]pyridine | | benzoic acid methyl | | | |
| (A, C, F, G) | | ester | | | |
| 4-Bromo-2- | 2,6-Dimethylphenol | 4-(2,6-Dimethyl- | 413 | 1.79 min (l) | 322.4 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |
| 4-Bromo-2- | 3-Ethyl-phenol | 4-(3-Ethyl-phenoxy)- | 414 | 1.83 min (l) | 322.4 (M + H)⁻ |
| (1H-tetrazol-5- | | 2-(1H-tetrazol-5-yl)- | | | |
| yl)-thieno | | thieno[2,3-c]pyridine | | | |
| [2,3-c]pyridine | | | | | |
| (A, C, F, G) | | | | | |
| 4-Bromo-2- | m-Cresol | 2-(1H-Tetrazol-5-yl)- | 415 | 1.73 min (l) | 308.4 (M + H)⁻ |
| (1H-tetrazol-5- | | 4-m-tolyloxy- | | | |
| yl)-thieno | | thieno[2,3-c]pyridine | | | |
| [2,3-c]pyridine | | | | | |
| (A, C, F, G) | | | | | |
| 4-Bromo-2- | 1-(3- | 4-(3-Piperazin-1-yl- | 416 | 1.52 min (l) | 378.4 (M + H)⁻ |
| (1H-tetrazol-5- | Hydroxyphenyl)- | phenoxy)-2-(1H- | | | |
| yl)-thieno | piperazine | tetrazol-5-yl)- | | | |
| [2,3-c]pyridine | | thieno[2,3-c]pyridine | | | |
| (A, C, F, G) | | | | | |
| 4-Bromo-2- | 3-Piperidinophenol | 4-(3-Piperidin-4-yl- | 417 | 1.56 min (l) | 377.5 (M + H)⁻ |
| (1H-tetrazol-5- | | phenoxy)-2-(1H- | | | |
| yl)-thieno | | tetrazol-5-yl)- | | | |
| [2,3-c]pyridine | | thieno[2,3-c]pyridine | | | |
| (A, C, F, G) | | | | | |

Other compounds obtained using general procedure U are shown (Table 17).

TABLE 17
Examples synthesized using general procedure U

| | | | HPLC Rₜ | |
|---|---|---|---|---|
| Amide Precursor | Product | Ex # | (Method) | m/z |
| 4-(4-Iodo-phenoxy)- | 4-(4-Iodo- | 417A | 12.63 min | 354.0(M+H)⁺ |
| thieno[2,3- | phenoxy)- | | (a) | |
| c]pyridine-2- | thieno[2,3- | | | |
| carboxylic acid | c]pyridine | | | |
| (A, D, U) | | | | |

Other compounds obtained using general procedure W are shown (Table 18).

TABLE 18
Examples synthesized using General Procedure W

| | Starting | | | | |
|---|---|---|---|---|---|
| Aldehyde | Material | | | HPLC Rₜ | |
| Precursor | amine | Product | Ex # | (Method) | m/z |
| 4-(3-Formyl- | 2- | 4-[3- | 418 | 2.94 min (i) | 410.3 (M + H)⁺ |
| phenyl)- | Naphthylamine | (Naphthalen-2- | | | |
| thieno[2,3- | | ylaminomethyl)- | | | |
| c]pyridine-2- | | phenyl]- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 3- | 4-{3-[(3- | 419 | 1.95 min (i) | 374.1 (M − H)⁺ |
| phenyl)- | Aminophenol | Hydroxy- | | | |
| thieno[2,3- | | phenylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 6- | 4-[3-(Quinolin- | 420 | 2.07 min (i) | 411.2 (M + H)⁺ |
| phenyl)- | Aminoquinoline | 6- | | | |
| thieno[2,3- | | ylaminomethyl)- | | | |
| c]pyridine-2- | | phenyl]- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 4-tert- | 4-{3-[(4-tert- | 421 | 3.57 min (i) | 416.0 (M + H)⁺ |
| phenyl)- | Butylaniline | Butyl- | | | |
| thieno[2,3- | | phenylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 3-Chloro-4- | 4-{3-[(3-Chloro- | 422 | 3.05 min (i) | 412.0 (M + H)⁺ |
| phenyl)- | fluoroaniline | 4-fluoro- | | | |
| thieno[2,3- | | phenylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | Benzylamine | 4-[3- | 423 | 7.41 min (a) | 374.1 (M + H)⁺ |
| phenyl)- | | (Benzylamino- | | | |
| thieno[2,3- | | methyl)-phenyl]- | | | |
| c]pyridine-2- | | thieno[2,3- | | | |
| carboxylic | | c]pyridine-2- | | | |
| acid amide | | carboxylic acid | | | |
| (A, C, J, W) | | amide | | | |
| 4-(3-Formyl- | 2- | 4-(3-{[(Pyridin- | 424 | 1.50 min (i) | 375.0 (M + H)⁺ |
| phenyl)- | (Aminomethyl) | 2-ylmethyl)- | | | |
| thieno[2,3- | pyridine | amino]-methyl}- | | | |
| c]pyridine-2- | | phenyl)- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 2,2- | 4-{3-[(2,2- | 425 | 3.05 min (i) | 464.0 (M + H)⁺ |
| phenyl)- | Diphenylethylamine | Diphenyl- | | | |
| thieno[2,3- | | ethylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 2- | 4-{3-[(2- | 426 | 2.38 min (i) | 376.0 (M + H)⁺ |
| phenyl)- | Aminophenol | Hydroxy- | | | |
| thieno[2,3- | | phenylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 3- | 4-{3-[(3- | 427 | 3.32 min (i) | 466.0 (M + H)⁺ |
| phenyl)- | Benzyloxyaniline | Benzyloxy- | | | |
| thieno[2,3- | | phenylamino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | Aniline | 4-(3- | 428 | 9.89 min (a) | 360.2 (M − H)⁺ |
| phenyl)- | | Phenylaminomethyl- | | | |
| thieno[2,3- | | phenyl)- | | | |
| c]pyridine-2- | | thieno[2,3- | | | |
| carboxylic | | c]pyridine-2- | | | |
| acid amide | | carboxylic acid | | | |
| (A, C, J, W) | | amide | | | |
| 4-(3-Formyl- | Indoline | 4-[3-(2,3- | 429 | 11.04 min (a) | 386.3 (M + H)⁺ |
| phenyl)- | | Dihydro-indol-1- | | | |
| thieno[2,3- | | ylmethyl)- | | | |
| c]pyridine-2- | | phenyl]- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | Aminomethylcyclohexane | 4-{3- | 430 | 2.05 min (i) | 378.0 (M − H)⁺ |
| phenyl)- | | [(Cyclohexylmethyl- | | | |
| thieno[2,3- | | amino)- | | | |
| c]pyridine-2- | | methyl]-phenyl}- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | Phenethylamine | 4-[3- | 431 | 7.82 min (a) | 388.2 (M + H)⁺ |
| phenyl)- | | (Phenethylamino- | | | |
| thieno[2,3- | | methyl)- | | | |
| c]pyridine-2- | | phenyl]- | | | |
| carboxylic | | thieno[2,3- | | | |
| acid amide | | c]pyridine-2- | | | |
| (A, C, J, W) | | carboxylic acid | | | |
| | | amide | | | |
| 4-(3-Formyl- | 1-(2- | Acetate1-{2-[3- | 432 | 6.44 min (a) | 381.3 (M + H)⁺ |
| phenyl)- | Aminoethyl) | (2-carbamoyl- | | | |
| thieno[2,3- | pyrrolidine | thieno[2,3- | | | |
| c]pyridine-2- | | c]pyridin-4-yl)- | | | |
| carboxylic | | benzylamino]- | | | |
| acid amide | | ethyl}- | | | |
| (A, C, J, W) | | pyrrolidinium | | | |
| 4-(3-Formyl- | 2-(2- | Acetate2-{2-[3- | 433 | 3.32 min (a) | 395.3 (M + H)⁺ |
| phenyl)- | Aminoethyl)- | (2-carbamoyl- | | | |
| thieno[2,3- | 1- | thieno[2,3- | | | |
| c]pyridine-2- | methylpyrrolidine | c]pyridin-4-yl)- | | | |
| carboxylic | | benzylamino]- | | | |
| acid amide | | ethyl}-1-methyl- | | | |
| (A, C, J, W) | | pyrrolidinium | | | |
| 4-(3-Formyl- | 4-(2- | Acetate4-{2-[3- | 434 | 6.41 min (a) | 389.1 (M + H)⁺ |
| phenyl)- | Aminoethyl) | (2-carbamoyl- | | | |
| thieno[2,3- | pyridine | thieno[2,3- | | | |
| c]pyridine-2- | | c]pyridin-4-yl)- | | | |
| carboxylic | | benzylamino]- | | | |
| acid amide | | ethyl}- | | | |
| (A, C, J, W) | | pyridinium | | | |
| 4-(Biphenyl- | 1- | Biphenyl-4-yl- | 435 | 9.336 min (a) | 415 (M + H)⁺ |
| 4-ylamino)- | Methylpiperazine | [2-(4-methyl- | | | |
| thieno[2,3-c] | | piperazin- | | | |
| pyridine-2- | | 1-ylmethyl)- | | | |
| carbaldehyde | | thieno[2,3- | | | |
| | | c]pyridin- | | | |
| | | 4-yl]-amine | | | |
| Benzaldehyde | (1S,4S)-4- | 4-((1S,4S)-5- | 436 | 6.43 min (a) | 365.2 (M + H)⁺ |
| | (2,5-Diaza- | Benzyl-2,5- | | | |
| | bicyclo[2.2.1] | diaza-bicyclo | | | |
| | hept-2-yl)- | [2.2.1]hept-2- | | | |
| | thieno[2,3- | yl)-thieno[2,3- | | | |
| | c]pyridine- | c]pyridine- | | | |
| | 2- | 2- | | | |
| | carboxylic | carboxylic acid | | | |
| | acid amide | amide | | | |
| | (GG(H, A, | | | | |
| | C, F, I) | | | | |
| Benzaldehyde | 4-((R)- | 4-((R)-1-Benzyl- | 437 | 6.71 min (a) | 353.2 (M + H)⁺ |
| | Pyrrolidin- | pyrrolidin-3- | | | |
| | 3-ylamino)- | ylamino)- | | | |
| | thieno | thieno[2,3- | | | |
| | [2,3- | c]pyridine-2- | | | |
| | c]pyridine- | carboxylic | | | |
| | 2- | acid amide | | | |
| | carboxylic | | | | |
| | acid amide | | | | |
| | (GG | | | | |
| | (H, A, C, F, | | | | |
| | I) | | | | |
| 2-Methyl- | 4- | 4-[1-(2-Methyl- | 438 | 7.15 min (a) | 381.2 (M + H)⁺ |
| benzaldehyde | (Piperidin- | benzyl)- | | | |
| | 4-ylamino)- | piperidin-4- | | | |
| | thieno[2,3- | ylamino]- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| 3-Methyl- | 4- | 4-[1-(3-Methyl- | 439 | 7.70 min (a) | 381.2 (M + H)⁺ |
| benzaldehyde | (Piperidin- | benzyl)- | | | |
| | 4-ylamino)- | piperidin-4- | | | |
| | thieno[2,3- | ylamino]- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| 4-Methyl- | 4- | 4-[1-(4-Methyl- | 440 | 7.36 min (a) | 381.1 (M + H)⁺ |
| benzaldehyde | (Piperidin- | benzyl)- | | | |
| | 4-ylamino)- | piperidin-4- | | | |
| | thieno[2,3- | ylamino]- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| 2-Formyl- | 4- | 4-[1-(2-Cyano- | 441 | 6.91 min (a) | 392.1 (M + H)⁺ |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| | (GG(H, A, | | | | |
| | C, F, I) | | | | |
| 3-Formyl- | 4- | 4-[1-(3-Cyano- | 442 | 6.64 min (a) | 392.1 (M + H)⁺ |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| | (GG(H, A, | | | | |
| | C, F, I) | | | | |
| 4-Formyl- | 4- | 4-[1-(4-Cyano- | 443 | 6.69 min (a) | 392.0 (M + H)⁺ |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| | (GG(H, A, | | | | |
| | C, F, I) | | | | |
| Cyclohexanecarbaldehyde | 4- | 4-(1- | 444 | 7.38 min (a) | 373.2 (M + H)⁺ |
| | (Piperidin- | Cyclohexylmethyl- | | | |
| | 4-ylamino)- | piperidin-4-ylamino)- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| | (GG(H, A, | | | | |
| | C, F, I) | | | | |
| Phenyl- | 4- | 4-(1-Phenethyl- | 445 | 7.16 min (a) | 381.2 (M + H)⁺ |
| acetaldehyde | (Piperidin- | piperidin-4- | | | |
| | 4-ylamino)- | ylamino)- | | | |
| | thieno[2,3- | thieno[2,3- | | | |
| | c]pyridine- | c]pyridine-2- | | | |
| | 2- | carboxylic | | | |
| | carboxylic | acid amide | | | |
| | acid amide | | | | |
| | (GG(H, A, | | | | |
| | C, F, I) | | | | |
| Pyridine-2- | 4- | 4-(1-Pyridin-2- | 446 | 5.68 min (a) | 368.1 (M + H)⁺ |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| Pyridine-3- | 4- | 4-(1-Pyridin-3- | 447 | 5.45 min (a) | 368.1 (M + H)⁺ |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| Pyridine-4- | 4- | 4-(1-Pyridin-4- | 448 | 5.47 min (a) | 368.2 (M + H)⁺ |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| | (GG(H, A, | | | | |
| | C, F, I) | | | | |
| 3H-Imidazole- | 4- | 4-[1-(3H- | 449 | 4.67 min (a) | 357.1 (M + H)⁺ |
| 4- | (Piperidin- | Imidazol-4- | | | |
| carbaldehyde | 4-ylamino)- | ylmethyl)-piperidin- | | | |
| | thieno[2,3- | 4-ylamino]- | | | |
| | c]pyridine- | thieno[2,3- | | | |
| | 2- | c]pyridine- | | | |
| | carboxylic | 2-carboxylic | | | |
| | acid amide | acid amide | | | |
| | (GG(H, A, | | | | |
| | C, F, I) | | | | |
| 1-Methyl-1H- | 4- | 4-[1-(1-Methyl- | 450 | 7.01 min (a) | 421.2 (M + H)⁺ |
| benzoimidazole- | (Piperidin- | 1H- | | | |
| 2-carbaldehyde | 4-ylamino)- | benzoimidazol- | | | |
| | thieno[2,3- | 2-ylmethyl)- | | | |
| | c]pyridine- | piperidin-4- | | | |
| | 2- | ylamino]-thieno | | | |
| | carboxylic | [2,3-c]pyridine- | | | |
| | acid amide | 2-carboxylic acid | | | |
| | (GG(H, A, | amide | | | |
| | C, F, I) | | | | |
| 1-Oxy- | 4- | 4-[1-(1-Oxy- | 451 | 5.00 min (a) | 384.1 (M + H)⁺ |
| pyridine-4- | (Piperidin- | pyridin-4- | | | |
| carbaldehyde | 4-ylamino)- | ylmethyl)-piperidin- | | | |
| | thieno[2,3- | 4- | | | |
| | c]pyridine- | ylamino]- | | | |
| | 2- | thieno[2,3-c]pyridine- | | | |
| | carboxylic | 2- | | | |
| | acid amide | carboxylic acid | | | |
| | (GG(H, A, | amide | | | |
| | C, F, I) | | | | |
| 5- | 4- | 4-[1-(5- | 452 | 5.35 min (a) | 387.1 (M + H)⁺ |
| Hydroxymethyl- | (Piperidin- | Hydroxymethyl- | | | |
| furan-2- | 4-ylamino)- | furan-2-ylmethyl)- | | | |
| carbaldehyde | thieno[2,3- | piperidin-4- | | | |
| | c]pyridine- | ylamino]- | | | |
| | 2- | thieno[2, | | | |
| | carboxylic | 3-c]pyridine-2- | | | |
| | acid amide | carboxylic acid | | | |
| | (GG(H, A, | amide | | | |
| | C, F, I) | | | | |
| 3-Methoxy- | 4- | 4-[1-(3- | 453 | 7.22 min (a) | 397.2 (M + H)⁺ |
| benzaldehyde | (Piperidin- | Methoxy- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2-carboxylic | | | |
| | acid amide | acid | | | |
| | (GG(H, A, | amide | | | |
| | C, F, I) | | | | |
| 4-Formyl- | 4- | 4-[1-(4-Cyano- | 453A | 6.69 (a) | 392.0 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| 3-Formyl- | 4- | 4-[1-(3-Cyano- | 453B | 6.64 (a) | 392.1 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| 2-Formyl- | 4- | 4-[1-(2-Cyano- | 453C | 6.91 (a) | 392.1 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| 4-Methyl- | 4- | 4-[1-(4-Methyl- | 453D | 7.36 (a) | 381.1 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| 3-Methyl- | 4- | 4-[1-(3-Methyl- | 453E | 7.70 (a) | 381.2 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| 2-Methyl- | 4- | 4-[1-(2-Methyl- | 453F | 7.15 (a) | 381.2 (M + H) |
| benzonitrile | (Piperidin- | benzyl)- | | | |
| | 4-ylamino)- | piperidin-4-ylamino]- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| Cyclohexanecarbaldehyde | 4- | 4-(1- | 453G | 7.38 (a) | 373.2 (M + H) |
| | (Piperidin- | Cyclohexylmethyl- | | | |
| | 4-ylamino)- | piperidin-4-ylamino)- | | | |
| | thieno[2,3- | thieno[2,3- | | | |
| | c]pyridine- | c]pyridine-2-carboxylic | | | |
| | 2- | acid | | | |
| | carboxylic | amide | | | |
| | acid amide | | | | |
| Pyridine-3- | 4- | 4-(1-Pyridin-3- | 453H | 5.45 (a) | 368.1 (M + H) |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| Phenyl- | 4- | 4-(1-Phenethyl- | 453I | 7.16 (a) | 381.2 (M + H) |
| acetaldehyde | (Piperidin- | piperidin-4- | | | |
| | 4-ylamino)- | ylamino)- | | | |
| | thieno[2,3- | thieno[2,3- | | | |
| | c]pyridine- | c]pyridine-2- | | | |
| | 2- | carboxylic | | | |
| | carboxylic | acid amide | | | |
| | acid amide | | | | |
| 1-Oxy- | 4- | 4-[1-(1-Oxy- | 453J | 5.00 (a) | 384.1 (M + H) |
| pyridine-4- | (Piperidin- | pyridin-4- | | | |
| carbaldehyde | 4-ylamino)- | ylmethyl)-piperidin- | | | |
| | thieno[2,3- | 4- | | | |
| | c]pyridine- | ylamino]- | | | |
| | 2- | thieno[2,3-c]pyridine- | | | |
| | carboxylic | 2- | | | |
| | acid amide | carboxylic acid | | | |
| | | amide | | | |
| 1-Methyl-1H- | 4- | 4-[1-(1-Methyl- | 453K | 7.01 (a) | 421.2 (M + H) |
| benzoimidazole- | (Piperidin- | 1H- | | | |
| 2-carbaldehyde | 4-ylamino)- | benzoimidazol- | | | |
| | thieno[2,3- | 2-ylmethyl)- | | | |
| | c]pyridine- | piperidin-4- | | | |
| | 2- | ylamino]-thieno | | | |
| | carboxylic | [2,3-c]pyridine- | | | |
| | acid amide | 2-carboxylic acid | | | |
| | | amide | | | |
| 3-Methoxy- | 4- | 4-[1-(3- | 453L | 7.22 (a) | 397.2 (M + H) |
| benzaldehyde | (Piperidin- | Methoxy- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2-carboxylic | | | |
| | acid amide | acid | | | |
| | | amide | | | |
| Pyridine-4- | 4- | 4-(1-Pyridin-4- | 453M | 5.47 (a) | 368.2 (M + H) |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| Pyridine-2- | 4- | 4-(1-Pyridin-2- | 453N | 5.68 (a) | 368.1 (M + H) |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2-carboxylic | | | |
| | carboxylic | acid | | | |
| | acid amide | amide | | | |
| 5- | 4- | 4-[1-(5- | 453O | 5.35 (a) | 387.1 (M + H) |
| Hydroxymethyl- | (Piperidin- | Hydroxymethyl- | | | |
| furan-2- | 4-ylamino)- | furan-2-ylmethyl)- | | | |
| carbaldehyde | thieno[2,3- | piperidin-4- | | | |
| | c]pyridine- | ylamino]- | | | |
| | 2- | thieno[2, | | | |
| | carboxylic | 3-c]pyridine-2- | | | |
| | acid amide | carboxylic acid | | | |
| | | amide | | | |
| 3H-Imidazole- | 4- | 4-[1-(3H- | 453P | 4.67 (a) | 357.1 (M + H) |
| 4- | (Piperidin- | Imidazol-4- | | | |
| carbaldehyde | 4-ylamino)- | ylmethyl)-piperidin- | | | |
| | thieno[2,3- | 4-ylamino]- | | | |
| | c]pyridine- | thieno[2,3- | | | |
| | 2- | c]pyridine- | | | |
| | carboxylic | 2-carboxylic | | | |
| | acid amide | acid amide | | | |
| 4-Methoxy- | 4- | 4-[1-(4- | 453Q | 6.74 (a) | 397.2 (M + H) |
| benzaldehyde | (Piperidin- | Methoxy- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2-carboxylic | | | |
| | acid amide | acid | | | |
| | | amide | | | |
| Benzo[1,3]dioxole- | 4- | 4-(1- | 453R | 6.61 (a) | 411.2 (M + H) |
| 5- | (Piperidin- | Benzo[1,3]dioxol- | | | |
| carbaldehyde | 4-ylamino)- | 5-ylmethyl-piperidin- | | | |
| | thieno[2,3- | 4- | | | |
| | c]pyridine- | ylamino)- | | | |
| | 2- | thieno[2,3-c]pyridine- | | | |
| | carboxylic | 2- | | | |
| | acid amide | carboxylic acid | | | |
| | | amide | | | |
| Benzaldehyde | (1S,4S)-4- | 4-((1S,4S)-5- | 453S | 6.43 (a) | 365.2 (M + H)+ |
| | (2,5-Diaza- | Benzyl-2,5- | | | |
| | bicyclo[2.2. | diaza-bicyclo | | | |
| | 1]hept-2- | [2.2.1]hept-2- | | | |
| | yl)- | yl)-thieno[2,3- | | | |
| | thieno[2,3- | c]pyridine- | | | |
| | c]pyridine- | 2- | | | |
| | 2- | carboxylic acid | | | |
| | carboxylic | amide | | | |
| | acid amide | | | | |
| Benzaldehyde | 4-((R)- | 4-((R)-1-Benzyl- | 453T | 6.71 (a) | 353.2 (M + H)+ |
| | Pyrrolidin- | pyrrolidin-3- | | | |
| | 3-ylamino)- | ylamino)- | | | |
| | thieno[2,3- | thieno[2,3- | | | |
| | c]pyridine- | c]pyridine-2- | | | |
| | 2- | carboxylic | | | |
| | carboxylic | acid amide | | | |
| | acid amide | | | | |
| 5-(2-Chloro- | 4- | 4-{1-[5-(2- | 453U | 1.91 (a) | 467 (M + H) |
| phenyl)-furan- | (Piperidin- | Chloro-phenyl)- | | | |
| 2- | 4-ylamino)- | furan-2- | | | |
| carbaldehyde | thieno[2,3- | ylmethyl]- | | | |
| | c]pyridine- | piperidin-4- | | | |
| | 2- | ylamino}- | | | |
| | carboxylic | thieno[2,3- | | | |
| | acid amide | c]pyridine-2- | | | |
| | | carboxylic acid | | | |
| | | amide | | | |
| 4-Benzyloxy- | 4- | 4-[1-(4- | 453V | 1.75 (a) | 473 (M + H) |
| benzaldehyde | (Piperidin- | Benzyloxy- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic acid | | | |
| | | amide | | | |
| Benzo[1,3]dioxole- | 4- | 4-(1- | 453W | 1.21 (a) | 411 (M + H) |
| 5- | (Piperidin- | Benzo[1,3]dioxol- | | | |
| carbaldehyde | 4-ylamino)- | 5-ylmethyl- | | | |
| | thieno[2,3- | piperidin-4- | | | |
| | c]pyridine- | ylamino)- | | | |
| | 2- | thieno[2,3- | | | |
| | carboxylic | c]pyridine-2- | | | |
| | acid amide | carboxylic acid | | | |
| | | amide | | | |
| 3,4-Dimethyl- | 4- | 4-[1-(3,4- | 453X | 1.49 (a) | 395 (M + H) |
| benzaldehyde | (Piperidin- | Dimethyl- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic acid | | | |
| | | amide | | | |
| Quinoline-2- | 4- | 4-(1-Quinolin-2- | 453Y | 1.42 (a) | 418 (M + H) |
| carbaldehyde | (Piperidin- | ylmethyl- | | | |
| | 4-ylamino)- | piperidin-4- | | | |
| | thieno[2,3- | ylamino)- | | | |
| | c]pyridine- | thieno[2,3- | | | |
| | 2- | c]pyridine-2- | | | |
| | carboxylic | carboxylic acid | | | |
| | acid amide | amide | | | |
| 3,5-Dichloro- | 4- | 4-[1-(3,5- | 453Z | 2.03 (a) | 435, 437 |
| benzaldehyde | (Piperidin- | Dichloro- | | | (M + H) |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 2,5-Difluoro- | 4- | 4-[1-(2,5- | 453AA | 1.61 (a) | 403 (M + H) |
| benzaldehyde | (Piperidin- | Difluoro- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 3,5-Dimethyl- | 4- | 4-[1-(3,5- | 453BB | 1.65 (a) | 395 (M + H) |
| benzaldehyde | (Piperidin- | Dimethyl- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 5-(3-Chlorophenyl)- | 4- | 4-{1-[5-(3- | 453CC | 2.04 (a) | 467 (M + H) |
| furan- | (Piperidin- | Chloro-phenyl)- | | | |
| 2- | 4-ylamino)- | furan-2- | | | |
| carbaldehyde | thieno[2,3- | ylmethyl]- | | | |
| | c]pyridine- | piperidin-4- | | | |
| | 2- | ylamino}- | | | |
| | carboxylic | thieno[2,3- | | | |
| | acid amide | c]pyridine-2- | | | |
| | | carboxylic | | | |
| 2,6-Difluoro- | 4- | 4-[1-(2,6- | 453DD | 1.55 (a) | 403 (M + H) |
| benzaldehyde | (Piperidin- | Difluoro- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 4-Phenoxy- | 4- | 4-[1-(4- | 453EE | 1.78 (a) | 459 (M + H) |
| benzaldehyde | (Piperidin- | Phenoxy- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 3,4-Dichloro- | 4- | 4-[1-(3,4- | 453FF | 1.85 (a) | 435, 437 |
| benzaldehyde | (Piperidin- | Dichloro- | | | (M + H) |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 5-(2- | 4- | 4-{1-[5-(2- | 453GG | 1.94 (a) | 501 (M + H) |
| Trifluoromethyl- | (Piperidin- | Trifluoromethyl- | | | |
| phenyl)- | 4-ylamino)- | phenyl)-furan-2- | | | |
| furan-2- | thieno[2,3- | ylmethyl]- | | | |
| carbaldehyde | c]pyridine- | piperidin-4- | | | |
| | 2- | ylamino}- | | | |
| | carboxylic | thieno[2,3- | | | |
| | acid amide | c]pyridine-2- | | | |
| | | carboxylic | | | |
| 2,3-Difluoro- | 4- | 4-[1-(2,3- | 453HH | 1.50 (a) | 403 (M + H) |
| benzaldehyde | (Piperidin- | Difluoro- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |
| 3,5-Difluoro- | 4- | 4-[1-(3,5- | 453II | 1.60 (a) | 403 (M + H) |
| benzaldehyde | (Piperidin- | Difluoro- | | | |
| | 4-ylamino)- | benzyl)- | | | |
| | thieno[2,3- | piperidin-4- | | | |
| | c]pyridine- | ylamino]- | | | |
| | 2- | thieno[2,3- | | | |
| | carboxylic | c]pyridine-2- | | | |
| | acid amide | carboxylic | | | |

Other compounds obtained using general procedure BB are shown (Table 19).

TABLE 19
Examples of General Procedure BB

| Ester | | | HPLC Rₜ | |
|---|---|---|---|---|
| Precursor | Product | Ex # | (Method) | m/z |
| 4-(Biphenyl- | 4-(Biphenyl-4- | 454 | 2.05 min (j) | 388(M+H)⁺ |
| 4-ylamino)-7- | ylamino)-7- | | | |
| cyano- | carbamimidoyl- | | | |
| thieno[2,3- | thieno[2,3- | | | |
| c]pyridine-2- | c]pyridine-2- | | | |
| carboxylic | carboxylic acid | | | |
| acid methyl | amide | | | |
| ester | | | | |
| 7-Amino4- | 7-Amino-4- | 455 | 2.39 min (i) | 346(M+H)⁺ |
| biphenyl-3-yl- | biphenyl-3-yl- | | | |
| thieno[2,3- | thieno[2,3- | | | |
| c]pyridine-2- | c]pyridine-2- | | | |
| carboxylic | carboxylic acid | | | |
| acid methyl ester | amide | | | |
| 4-biphenyl-3- | 4-biphenyl-3-yl- | 456 | 9.32 min (a) | 314.2(M−H)⁻ |
| yl-1H- | 1H-pyrrolo[2,3- | | | |
| pyrrolo[2,3- | c]pyridine-2- | | | |
| c]pyridine-2- | carboxylic acid | | | |
| carboxylic acid | amide | | | |
| methyl ester (Z, AA, J) | | | | |
| 4-(2- | 4-(Piperidin-4- | 457 | 4.71 min (a) | 277.2(M+H)⁺ |
| Carbamoyl- | ylamino)- | | | |
| thieno[2,3- | thieno[2,3- | | | |
| c]pyridin- | c]pyridine-2- | | | |
| 4-ylamino)- | carboxylic acid | | | |
| piperidine-1- | amide | | | |
| carboxylic | | | | |
| acid tert-butyl | | | | |
| ester (H(A, C, | | | | |
| F, I) | | | | |
| (R)-3-(2- | 4-((R)- | 458 | 4.87 min (a) | 263.2(M+H)⁺ |
| Carbamoyl- | Pyrrolidin-3- | | | |
| thieno[2,3- | ylamino)-thieno | | | |
| c]pyridin- | [2,3-c]pyridine- | | | |
| 4- | 2-carboxylic acid | | | |
| ylamino)- | amide | | | |
| pyrrolidine-1- | | | | |
| carboxylic | | | | |
| acid tert- | | | | |
| butyl ester | | | | |
| (H(A, C, F, I) | | | | |
| (1R,4S)-5-(2- | (1S, 4S)-4-(2,5- | 459 | 5.03 min (a) | 275.2(M+H)+ |
| Carbamoyl- | Diaza- | | | |
| thieno[2,3-c | bicyclo[2.2.1] | | | |
| ]pyridin-4-yl)- | hept-2-yl)- | | | |
| 2,5-diaza- | thieno[2,3- | | | |
| bicyclo[2.2.1 | c]pyridine-2- | | | |
| ]heptane-2- | carboxylic acid | | | |
| carboxylic | amide | | | |
| acid tert- | | | | |
| butyl ester | | | | |
| (H(A, C, F, I) | | | | |
| N-(4- | N-(4- | 459A | 6.22 min (t) | 386(M+H)⁺ |
| [2,2′]Bithiophenyl- | [2,2′]Bithiophenyl- | | | |
| 5-yl- | 5-yl- | | | |
| thieno[2,3- | thieno[2,3- | | | |
| c]pyridin-2- | c]pyridin-2-yl)- | | | |
| yl)-oxalamic | oxalamide | | | |
| acid ethyl | | | | |
| ester | | | | |
| (A, B, E, J, Q, | | | | |
| R, O) | | | | |
| 4-Bromo-7- | 7-Amino-4- | 459B | 0.77 min (a) | 272, 274(M+H)⁺ |
| chloro- | bromo- | | | |
| thieno[2,3- | thieno[2,3- | | | |
| c]pyridine-2- | c]pyridine-2- | | | |
| carboxylic | carboxylic acid | | | |
| acid amide | amide | | | |
| (TG10) | | | | |

Other compounds obtained using general procedure HH are shown (Table 20).

TABLE 20
Examples of compounds prepared using method HH

| | | | | HPLC | |
|---|---|---|---|---|---|
| Chloride | | | | Rₜ | |
| Precursor | Amine | Product | Ex# | (method) | m/z |
| 4-[4-(2-Chloro- | 1- | 4-{4-[2-(4-Methyl- | 460 | 6.38 min | 410 (M + H)⁺ |
| acetylamino)- | Methylpiperazine | piperazin-1-yl)- | | (a) | |
| phenyl]- | | acetylamino]- | | | |
| thieno[2,3- | | phenyl}-thieno[2,3- | | | |
| c]pyridine-2- | | c]pyridine-2- | | | |
| carboxylic acid | | carboxylic acid amide | | | |
| amide | | | | | |
| (A, C, J, HH) | | | | | |
| 4-[4-(2-Chloro- | 1H-Pyrazole | 4-[4-(2-Pyrazol-1-yl- | 461 | 7.53 min | 378 (M + H)⁺ |
| acetylamino)- | | acetylamino)- | | (a) | |
| phenyl]- | | phenyl]-thieno[2,3- | | | |
| thieno[2,3- | | c]pyridine-2- | | | |
| c]pyridine-2- | | carboxylic acid amide | | | |
| carboxylic acid | | | | | |
| amide | | | | | |
| (A, C, J, HH) | | | | | |
| 4-[4-(2-Chloro- | 1,2,4-Triazole | 4-[4-(2- | 462 | 6.83 min | 379 (M + H)⁺ |
| acetylamino)- | sodium | [1,2,4]Triazol-1-yl- | | (a) | |
| phenyl]- | derivative | acetylamino)- | | | |
| thieno[2,3- | | phenyl]-thieno[2,3- | | | |
| c]pyridine-2- | | c]pyridine-2- | | | |
| carboxylic acid | | carboxylic acid amide | | | |
| amide (A, C, J, | | | | | |
| HH) | | | | | |
| 4-(Piperidin-4- | 1-Fluoro-4- | 4-[1-(4-Nitro- | 462A | 5.42 (a) | 398 (M + H) |
| ylamino)- | nitro-benzene | phenyl)-piperidin-4-ylamino]- | | | |
| thieno[2,3- | | thieno[2,3- | | | |
| c]pyridine-2- | | c]pyridine-2-carboxylic | | | |
| carboxylic acid | | acid amide | | | |
| amide | | | | | |
| 4-(Piperidin-4- | 2-Chloro-4,6- | 4-[1-(4,6-Dimethoxy- | 462B | 5..63 (a) | 415.1 (M + H) |
| ylamino)- | dimethoxy- | pyrimidin-2-yl)- | | | |
| thieno[2,3- | pyrimidine | piperidin-4- | | | |
| c]pyridine-2- | | ylamino]-thieno[2,3- | | | |
| carboxylic acid | | c] | | | |
| amide | | pyridine-2-carboxylic | | | |
| | | acid amide | | | |

### General Procedure II: Suzuki coupling of a boronate or boronic acid with an aryl bromide or iodide substrate.

To a mixture of a boronate ester or a boronic acid (1-5 equivalents, preferably 2 equivalents), an aryl halide (for example, an aryl bromide, aryl chloride or an aryl iodide, preferably an aryl chloride) (preferably 1 equivalent) and an inorganic base (for example, sodium carbonate or cesium carbonate, preferably cesium carbonate) (6-16 equivalents, preferably 10 equivalents) in a degassed organic solvent (for example DME, DMF, 1,4-dioxane, or toluene, preferably DMF) is added a palladium catalyst (for example tetrakis(triphenylphosphine)palladium(0) or tris(dibenzylideneacetone)dipalladium(0) (0.01-0.10 equivalents, preferably 0.05 equivalents) and tri-tert-butylphosphine (0.1 to 0.5 equivalents, preferably 0.3). The reaction mixture is heated at about 50-100° C. (preferably about 80° C.) for about 2-24 hours (preferably about 18 hours) under an inert atmosphere. The reaction mixture is allowed to cool to ambient temperature and filtered. The solvents are removed under reduced pressure to afford the product that can be further purified by chromatography or crystallization.

Illustration of General Procedure II

### Preparation #35: 4-(3-Pyridin-3-yl-phenyl)-2-(1H-tetrazol-5-yl)-thieno[2,3-c]pyridine

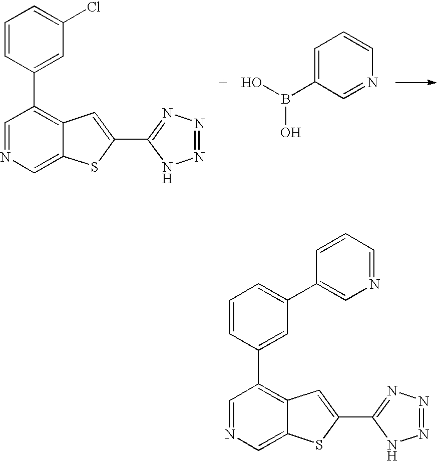

To a mixture of 4-(3-chloro-phenyl)-2-(1H-tetrazol-5-yl)-thieno[2,3-c]pyridine (prepared using general procedures A, C, F, G, J) (0.050 g, 0.16 mmol), pyridine-3-boronic acid (0.059 g, 0.48 mmol), and aqueous cesium carbonate (2N, 0.50 mL, 1.0 mmol) in degassed dioxane (3.3 mL) was added tris(dibenzylideneacetone)dipalladium(0) (0.0073 g, 0.0080 mmol), and tri-tert-butylphosphine (10% by weight in hexane) (0.097 mL, 0.010 g, 0.047 mmol) at room temperature under an atmosphere of nitrogen. The reaction mixture was heated at about 80° C. for about 18 hours. The mixture was allowed to cool to ambient temperature, filtered through celite, and the solvents were removed under reduced pressure. The residue was purifed by preparative RP-HPLC (20%-100% acetonitrile/0.05M aqueous ammonium acetate, buffered to pH 4.5, over 35 min at 15 mL/min; λ=254 nm; Hypersil C18, 100 Å, 8 μm, 250×21.2 mm column) to give 4-(3-pyridin-3-yl-phenyl)-2-(]H-tetrazol-5-yl)-thieno[2,3-c]pyridine as a white solid (0.0029 g, 0.0081 mmol); ¹H NMR (d₆-DMSO, 400 MHz): δ 9.18 (1H, s), 8.48 (1H, s), 8.22 (2H, d), 8.17 (1H, s), 8.04 (1H, d), 7.87 (1H, s), 7.84 (3H, d), 7.67 (1H, t), 7.23 (2H, d); RP-HPLC (Table 1, Method m) Rₜ 3.48 min; m/z: (M+H)⁺ 372.

Other compounds obtained using the above procedure II are shown below (Table 21).

TABLE 21
Examples synthesized using procedure II

| Halide | Boronate | | | HPLC Rₜ | |
|---|---|---|---|---|---|
| Precursor | Precursor | Product | Ex # | (Method) | m/z |
| 4-(3-Chloro-4- | Phenylboronic | 2-(1H-Tetrazol-5- | 463 | 3.08 min | 424 (M + H)⁺ |
| trifluoromethyl- | acid | yl)-4-(6- | | (m) | |
| phenyl)-2- | | trifluoromethyl- | | | |
| (1H-tetrazol-5- | | biphenyl-3-yl)- | | | |
| yl)-thieno[2,3- | | thieno[2,3- | | | |
| c]pyridine | | c]pyridine | | | |
| (C, F, G, J) | | | | | |
| 4-(3-Chloro-4- | Phenylboronic | 4-(6-Fluoro- | 464 | 2.98 min | 374 (M + H)⁺ |
| fluoro-phenyl)- | acid | biphenyl-3-yl)-2- | | (m) | |
| 2-(1H-tetrazol- | | (1H-tetrazol-5-yl)- | | | |
| 5-yl)- | | thieno[2,3- | | | |
| thieno[2,3- | | c]pyridine | | | |
| c]pyridine | | | | | |
| (C, F, G, J) | | | | | |
| 4-(3-Chloro-4- | Phenylboronic | 4-(6-Methoxy- | 465 | 2.88 min | 386 (M + H)⁺ |
| methoxyphenyl)- | acid | biphenyl-3-yl)-2- | | (m) | |
| 2-(1H- | | (1H-tetrazol-5-yl)- | | | |
| tetrazol-5-yl)- | | thieno[2,3- | | | |
| thieno[2,3- | | c]pyridine | | | |
| c]pyridine | | | | | |
| (C, F, G, J) | | | | | |
| 4-(3-Chloro- | 3- | 4-(6-Methoxy- | 466 | 2.66 min | 433 (M + H)⁺ |
| phenyl)-2-(1H- | (Methanesulfonyl) | biphenyl-3-yl)-2- | | (m) | |
| tetrazol-5-yl)- | phenylboronic | (1H-tetrazol-5-yl)- | | | |
| thieno[2,3- | acid | thieno[2,3- | | | |
| c]pyridine | | c]pyridine | | | |
| (C, F, G, J) | | | | | |
| 4-(3-Chloro- | 3- | 4-(3′-Methoxy- | 467 | 2.91 min | 386 (M + H)⁺ |
| phenyl)-2-(1H- | Methoxyphenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 2- | 4-(3′-Methoxy- | 468 | 2.91 min | 386 (M + H)⁺ |
| phenyl)-2-(1H- | Methoxyphenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 2- | 4-(2′-Methyl- | 469 | 1.95 min | 368 (M − H)⁻ |
| phenyl)-2-(1H- | Methylphenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 3- | 4-(3′-Nitro- | 470 | 1.98 min | 399 (M − H)⁻ |
| phenyl)-2-(1H- | Nitrophenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 3- | 4-(3′-Methyl- | 471 | 2.02 min | 368 (M − H)⁻ |
| phenyl)-2-(1H- | Nethylphenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 4- | 4-(4′-Fluoro- | 472 | 1.95 min | 372 (M − H)⁻ |
| phenyl)-2-(1H- | Fluorophenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 4- | 4-(4′-Methyl- | 473 | 1.99 min | 368 (M − H)⁻ |
| phenyl)-2-(1H- | Methylphenylboronic- | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| (C, F, G, J) | | | | | |
| 4-(3-Chloro- | 3,5- | 4-[3-(3,5-Dimethylisoxazol- | 474 | 1.68 min | 373 (M − H)⁻ |
| phenyl)-2-(1H- | Dimethylisoxazole- | 4-yl)- | | (m) | |
| tetrazol-5-yl)- | 4-boronic | phenyl]-2-(1H- | | | |
| thieno[2,3- | acid | tetrazol-5-yl)- | | | |
| c]pyridine | | thieno[2,3 | | | |
| | | c]pyridine | | | |
| 4-(3-Chloro- | 4- | 3′-[2-(1H-Tetrazol- | 475 | 1.85 min | 379 (M − H)⁻ |
| phenyl)-2-(1H- | Cyanophenylboronic- | 5-yl)-thieno[2,3- | | (m) | |
| tetrazol-5-yl)- | acid | c]pyridin-4-yl]- | | | |
| thieno[2,3- | | biphenyl-4- | | | |
| c]pyridine | | carbonitrile | | | |
| 4-(3-Chloro- | 4- | 4-(4′- | 476 | 1.7 min | 432 (M − H)⁻ |
| phenyl)-2-(1H- | Methanesulfonyl)- | Methanesulfonyl- | | (m) | |
| tetrazol-5-yl)- | phenylboronic | biphenyl-3-yl)-2- | | | |
| thieno[2,3- | acid | (1H-tetrazol-5-yl)- | | | |
| c]pyridine | | thieno[2,3- | | | |
| | | c]pyridine | | | |
| 4-(3-Chloro- | 3- | 3′-[2-(1H-Tetrazol- | 477 | 1.69 min | 369 (M − H)⁻ |
| phenyl)-2-(1H- | Aminophenylboronic | 5-yl)-thieno[2,3- | | (m) | |
| tetrazol-5-yl)- | acid | c]pyridin-4-yl]- | | | |
| thieno[2,3- | | biphenyl-3-ylamine | | | |
| c]pyridine | | | | | |
| 4-(3-Chloro- | 2- | 4-(2′-Fluoro- | 478 | 1.91 min | 372 (M − H)⁻ |
| phenyl)-2-(1H- | Fluorophenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| 4-(3-Chloro- | 3- | 1-{3′-[2-(1H- | 479 | 1.81 min | 396 (M − H)⁻ |
| phenyl)-2-(1H- | Acetylphenylboronic | Tetrazol-5-yl)- | | (m) | |
| tetrazol-5-yl)- | acid | thieno[2,3- | | | |
| thieno[2,3- | | c]pyridin-4-yl]- | | | |
| c]pyridine | | biphenyl-3-yl}- | | | |
| | | ethanone | | | |
| 4-(3-Chloro- | 3- | 2-(1H-Tetrazol-5- | 480 | 2.07 min | 422 (M − H)⁻ |
| phenyl)-2-(1H- | (Trifluoromethyl)- | yl)-4-(3′- | | (m) | |
| tetrazol-5-yl)- | phenylboronic | trifluoromethyl- | | | |
| thieno[2,3- | acid | biphenyl-3-yl)- | | | |
| c]pyridine | | thieno[2,3- | | | |
| | | c]pyridine | | | |
| 4-(3-Chloro- | 4- | 1-{3′-[2-(1H- | 481 | 1.8 min | 396 (M − H)⁻ |
| phenyl)-2-(1H- | Acetylphenylboronic | Tetrazol-5-yl)- | | (m) | |
| tetrazol-5-yl)- | acid | thieno[2,3- | | | |
| thieno[2,3- | | c]pyridin-4-yl]- | | | |
| c]pyridine | | biphenyl-4-yl}- | | | |
| | | ethanone | | | |
| 4-(3-Chloro- | 4-(Trifluoromethyl)- | 2-(1H-Tetrazol-5- | 482 | 2.09 min | 422 (M − H)⁻ |
| phenyl)-2-(1H- | phenylboronic | yl)-4-4(4′- | | (m) | |
| tetrazol-5-yl)- | acid | trifluoromethyl- | | | |
| thieno[2,3- | | biphenyl-3-yl)- | | | |
| c]pyridine | | thieno[2,3- | | | |
| | | c]pyridine | | | |
| 4-(3-Chloro- | 3- | 4-(3′-Fluoro- | 483 | 1.93 min | 372 (M − H)⁻ |
| phenyl)-2-(1H- | Fluorophenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| 4-(3-Chloro- | 4-tert-Butyl- | 4-(4′-tert-Butyl- | 484 | 2.25 min | 410 (M − H)⁻ |
| phenyl)-2-(1H- | benzene- | biphenyl-3-yl)-2- | | (m) | |
| tetrazol-5-yl)- | boronic acid | (1H-tetrazol-5-yl)- | | | |
| thieno[2,3- | | thieno[2,3- | | | |
| c]pyridine | | c]pyridine | | | |
| 4-(3-Chloro- | 3,4- | 4-(3′,4′-Dimethoxy- | 485 | 1.81 min | 414 (M − H)⁻ |
| phenyl)-2-(1H- | Dimethoxy- | biphenyl-3-yl)-2- | | (m) | |
| tetrazol-5-yl)- | phenylboronic | (1H-tetrazol-5-yl)- | | | |
| thieno[2,3- | acid | thieno[2,3- | | | |
| c]pyridine | | c]pyridine | | | |
| 4-(3-Chloro- | 4-(N,N- | Dimethyl-{3′-[2- | 486 | 1.97 min | 397 (M − H)⁻ |
| phenyl)-2-(1H- | Dimethylamino)- | (1H-tetrazol-5-yl)- | | (m) | |
| tetrazol-5-yl)- | phenylboronic | thieno[2,3- | | | |
| thieno[2,3- | acid | c]pyridin-4-yl]- | | | |
| c]pyridine | | biphenyl-4-yl}- | | | |
| | | amine | | | |
| 4-(3-Chloro- | 4- | 4-(4′-Ethyl- | 487 | 2.1 min | 382 (M − H)⁻ |
| phenyl)-2-(1H- | Ethylphenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| 4-(3-Chloro- | 1- | 4-(3- | 488 | 2.06 min | 410 (M − H)⁻ |
| phenyl)-2-(1H- | Benzothiophen- | Benzo[b]thiophen- | | (m) | |
| tetrazol-5-yl)- | 3-yl- | 3-yl-phenyl)-2-(1H- | | | |
| thieno[2,3- | boronic acid | tetrazol-5-yl)- | | | |
| c]pyridine | | thieno[2,3- | | | |
| | | c]pyridine | | | |
| 4-(3-Chloro- | 4- | {3′-[2-(1H-Tetrazol- | 489 | 1.65 min | 384 (M − H)⁻ |
| phenyl)-2-(1H- | (Hydroxymethyl)- | 5-yl)-thieno[2,3- | | (m) | |
| tetrazol-5-yl)- | phenylboronic | c]pyridin-4-yl]- | | | |
| thieno[2,3- | acid | biphenyl-4-yl}- | | | |
| c]pyridine | | methanol | | | |
| 4-(3-Chloro- | 3- | 3′-[2-(1H-Tetrazol- | 490 | 1.83 min | 379 (M − H)⁻ |
| phenyl)-2-(1H- | Cyanophenylboronic | 5-yl)-thieno[2,3- | | (m) | |
| tetrazol-5-yl)- | acid | c]pyridin-4-yl]- | | | |
| thieno[2,3- | | biphenyl-3- | | | |
| c]pyridine | | carbonitrile | | | |
| 4-(3-Chloro- | 3- | N-{3′-[2-(1H- | 491 | 1.74 min | 447 (M − H)⁻ |
| phenyl)-2-(1H- | (Methylsulfonyl- | Tetrazol-5-yl)- | | (m) | |
| tetrazol-5-yl)- | amino) | thieno[2,3- | | | |
| thieno[2,3- | phenylboronic | c]pyridin-4-yl]- | | | |
| c]pyridine | acid | biphenyl-3-yl}- | | | |
| | | methanesulfonamide | | | |
| 4-(3-Chloro- | 3- | 3′-[2-(1H-Tetrazol- | 492 | 1.73 min | 370 (M − H)⁻ |
| phenyl)-2-(1H- | Hydroxyphenylboronic | 5-yl)-thieno[2,3- | | (m) | |
| tetrazol-5-yl)- | acid | c]pyridin-4-yl]- | | | |
| thieno[2,3- | | biphenyl-3-ol | | | |
| c]pyridine | | | | | |
| 4-(3-Chloro- | (2- | {3′-[2-(1H-Tetrazol- | 493 | 1.7 min | 384 (M − H)⁻ |
| phenyl)-2-(1H- | Hydroxymethyl- | 5-yl)-thieno[2,3- | | (m) | |
| tetrazol-5-yl)- | phenyl)boronic | c]pyridin-4-yl]- | | | |
| thieno[2,3- | acid | biphenyl-2-yl}- | | | |
| c]pyridine | dehydrate | methanol | | | |
| 4-(3-Chloro- | Furan-3- | 4-(3-Furan-3-yl- | 494 | 1.77 min | 344 (M − H)⁻ |
| phenyl)-2-(1H- | boronic acid | phenyl)-2-(1H- | | (m) | |
| tetrazol-5-yl)- | | tetrazol-5-yl)- | | | |
| thieno[2,3- | | thieno[2,3- | | | |
| c]pyridine | | c]pyridine | | | |
| 4-(3-Chloro- | 3- | 4-(3′- | 495 | 2.02 min | 400 (M − H)⁻ |
| phenyl)-2-(1H- | Thioanisoleboronic | Methylsulfanyl- | | (m) | |
| tetrazol-5-yl)- | acid | biphenyl-3-yl)-2- | | | |
| thieno[2,3- | | (1H-tetrazol-5-yl)- | | | |
| c]pyridine | | thieno[2,3- | | | |
| | | c]pyridine | | | |
| 4-(3-Chloro- | 2- | 3-{3-[2-(1H- | 496 | 1.67 min | 388 (M − H)⁻ |
| phenyl)-2-(1H- | Formylthiophene- | Tetrazol-5-yl)- | | (m) | |
| tetrazol-5-yl)- | 3-boronic | thieno[2,3- | | | |
| thieno[2,3- | acid | c]pyridin-4-yl]- | | | |
| c]pyridine | | phenyl}-thiophene- | | | |
| | | 2-carbaldehyde | | | |
| 4-(3-Chloro- | (3- | 3′-[2-(1H-Tetrazol- | 497 | 1.59 min | 397 (M − H)⁻ |
| phenyl)-2-(1H- | Aminocarbonylphenyl) | 5-yl)-thieno[2,3- | | (m) | |
| tetrazol-5-yl)- | boronic acid | c]pyridin-4-yl]- | | | |
| thieno[2,3- | | biphenyl-3- | | | |
| c]pyridine | | carboxylic acid | | | |
| | | amide | | | |
| 4-(3-Chloro- | 4-(4,4,5,5- | 3′-[2-(1H-Tetrazol- | 498 | 1.66 min | 369 (M − H)⁻ |
| phenyl)-2-(1H- | Tetramethyl- | 5-yl)-thieno[2,3- | | (m) | |
| tetrazol-5-yl)- | 1,3,2- | c]pyridin-4-yl]- | | | |
| thieno[2,3- | dioxaborolan- | biphenyl-4-ylamine | | | |
| c]pyridine | 2-yl) | | | | |
| | aniline | | | | |
| 4-(3-Chloro- | 2-(N,N- | Dimethyl-{3′-[2- | 499 | 1.66 min | 411 (M − H)⁻ |
| phenyl)-2-(1H- | Dimethylamino | (1H-tetrazol-5-yl)- | | (m) | |
| tetrazol-5-yl)- | methyl)phenylboronic | thieno[2,3- | | | |
| thieno[2,3- | acid | c]pyridin-4-yl]- | | | |
| c]pyridine | | biphenyl-2- | | | |
| | | ylmethyl}-amine | | | |
| 4-(3-Chloro- | 4- | 4-(4′-Nitro- | 500 | 1.91 min | 399 (M − H)⁻ |
| phenyl)-2-(1H- | Nitrophenylboronic | biphenyl-3-yl)-2- | | (m) | |
| tetrazol-5-yl)- | acid | (1H-tetrazol-5-yl)- | | | |
| thieno[2,3- | | thieno[2,3- | | | |
| c]pyridine | | c]pyridine | | | |
| 4-(3-Chloro- | 2-(4,4,5,5- | 3′-[2-(1H-Tetrazol- | 501 | 1.73 min | 369 (M − H)⁻ |
| phenyl)-2-(1H- | Tetramethyl- | 5-yl)-thieno[2,3- | | (m) | |
| tetrazol-5-yl)- | 1,3,2- | c]pyridin-4-yl]- | | | |
| thieno[2,3- | dioxaborolan- | biphenyl-2-ylamine | | | |
| c]pyridine | 2-yl) | | | | |
| | aniline | | | | |
| 4-(3-Chloro- | 3-(N,N- | Dimethyl-{3′-[2- | 502 | 1.98 min | 397 (M − H)⁻ |
| phenyl)-2-(1H- | Dimethylamino) | (1H-tetrazol-5-yl)- | | (m) | |
| tetrazol-5-yl)- | phenylboronic | thieno[2,3- | | | |
| thieno[2,3- | acid | c]pyridin-4-yl]- | | | |
| c]pyridine | | biphenyl-3-yl}- | | | |
| | | amine | | | |
| 4-(3-Chloro- | 3- | 4-(3′-Butoxy- | 503 | 2.27 min | 426 (M − H)⁻ |
| phenyl)-2-(1H- | Butoxyphenyl | biphenyl-3-yl)-2- | | (m) | |
| tetrazol-5-yl)- | boronic acid | (1H-tetrazol-5-yl)- | | | |
| thieno[2,3- | | thieno[2,3- | | | |
| c]pyridine | | c]pyridine | | | |
| 4-(3-Chloro- | (4-n- | 4-(4′-Butoxy- | 504 | 2.28 min | 426 (M − H)⁻. |
| phenyl)-2-(1H- | Butoxyphenyl) | biphenyl-3-yl)-2- | | (m) | |
| tetrazol-5-yl)- | boronic acid | (1H-tetrazol-5-yl)- | | | |
| thieno[2,3- | | thieno[2,3- | | | |
| c]pyridine | | c]pyridine | | | |
| 4-(3-Chloro- | 2-Methoxy- | 3-Methoxy-3′-[2- | 505 | 1.68 min | 400 (M − H)⁻ |
| phenyl)-2-(1H- | 4- | (1H-tetrazol-5-yl)- | | (m) | |
| tetrazol-5-yl)- | (4,4,5,5- | thieno[2,3- | | | |
| thieno[2,3- | tetramethyl- | c]pyridin-4-yl]- | | | |
| c]pyridine | 1,3,2- | biphenyl-4-ol | | | |
| | dioxaborolan- | | | | |
| | 2-yl)phenol | | | | |
| 7-Amino-4-(3- | 3- | 7-Amino-4-(3′- | 505A | 5.11 min | 424.12 (M + H)⁺ |
| chloro-phenyl)- | (Methanesulfonyl) | methanesulfonyl- | | (q) | |
| thieno[2,3- | phenylboronic | biphenyl-3-yl)- | | | |
| c]pyridine-2- | acid | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |
| 7-Amino-4-(3- | 1- | 7-Amino-4-(3- | 505B | 6.22 min | 402.29 (M + H)⁺ |
| chloro-phenyl)- | Benzothiephen- | benzo[b]thiophen-3- | | (q) | |
| thieno[2,3- | 3-ylboronic | yl-phenyl)- | | | |
| c]pyridine-2- | acid | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |
| 7-Amino-4-(3- | 4- | 7-Amino-4-(4′- | 505C | 6.35 min | 374.32 (M + H)⁺ |
| chloro-phenyl)- | Ethylphenylboronic | ethyl-biphenyl-3- | | (q) | |
| thieno[2,3- | acid | yl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | 3- | 7-Amino-4-(3′- | 505D | 6.04 min | 360.32 (M + H)⁺ |
| chloro-phenyl)- | Methylphenylboronic | methyl-biphenyl-3- | | (q) | |
| thieno[2,3- | acid | yl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | 3,4- | 7-Amino-4-(3′,4′- | 505E | 5.38 min | 406.23 (M + H)⁺ |
| chloro-phenyl)- | Dimethoxybenzeneboronic | dimethoxy- | | (q) | |
| thieno[2,3- | acid | biphenyl-3-yl)- | | | |
| c]pyridine-2- | | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |
| 7-Amino-4-(3- | 3- | 7-Amino-4-(3′- | 505F | 6.21 min | 414.30 (M + H)⁺ |
| chloro-phenyl)- | (Trifluoromethyl) | trifluoromethyl- | | (q) | |
| thieno[2,3- | phenylboronic | biphenyl-3-yl)- | | | |
| c]pyridine-2- | acid | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |
| 7-Amino-4-(3- | 3- | 7-Amino-4-(3′- | 505G | 5.06 min | 361.29 (M + H)⁺ |
| chloro-phenyl)- | Aminophenylboronic | amino-biphenyl-3- | | (q) | |
| thieno[2,3- | acid | yl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | Pyridine-4- | 7-Amino-4-(3- | 505H | 4.62 min | 347.32 (M + H)⁺ |
| chloro-phenyl)- | boronic acid | pyridin-4-yl- | | (q) | |
| thieno[2,3- | | phenyl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | (4- | 7-Amino-4-(4′- | 505I | 4.56 min | 389.28 (M + H)⁺ |
| chloro-phenyl)- | Aminocarbonylphenyl) | carbamoyl- | | (q) | |
| thieno[2,3- | boronic | biphenyl-3-yl)- | | | |
| c]pyridine-2- | acid | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |
| 7-Amino-4-(3- | 4- | 4-(4′-Acetyl- | 505J | 5.37 min | 388.30 (M + H)⁺ |
| chloro-phenyl)- | Acetylphenylboronic | biphenyl-3-yl)-7- | | (q) | |
| thieno[2,3- | acid | amino-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | 4- | 7-Amino-4-(4′- | 505K | 5.71 min | 376.29 (M + H)⁺ |
| chloro-phenyl)- | Methoxyphenylboronic | methoxy-biphenyl- | | (q) | |
| thieno[2,3- | acid | 3-yl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | 4- | 7-Amino-4-(4′- | 505L | 5.51 min | 371.28 (M + H)⁺ |
| chloro-phenyl)- | Cyanophenylboronic | cyano-biphenyl-3- | | (q) | |
| thieno[2,3- | acid | yl)-thieno[2,3- | | | |
| c]pyridine-2- | | c]pyridine-2- | | | |
| carboxylic acid | | carboxylic acid | | | |
| amide | | amide | | | |
| (A, B, OO, J, AAA, | | | | | |
| I, BB) | | | | | |
| 7-Amino-4-(3- | 3- | 7-Amino-4-(3′- | 505M | 5.11 min | 424.12 (M + H)⁺ |
| chloro-phenyl)- | (Methanesulfonyl) | methanesulfonyl- | | (q) | |
| thieno[2,3- | phenylboronic | biphenyl-3-yl)- | | | |
| c]pyridine-2- | acid | thieno[2,3- | | | |
| carboxylic acid | | c]pyridine-2- | | | |
| amide | | carboxylic acid | | | |
| (A, B, OO, J, AAA, | | amide | | | |
| I, BB) | | | | | |

### General Procedure JJ: Suzuki coupling of a boronate or boronic acid with an aryl iodide substrate.

To a mixture of a boronate ester or a boronic acid (1-5 equivalents, preferably 2 equivalents), an aryl halide (for example, an aryl bromide or an aryl iodide, preferably an aryl iodide) (preferably 1 equivalent) and an inorganic base (for example, sodium carbonate or cesium carbonate, preferably cesium carbonate) (6-16 equivalents, preferably 10 equivalents) in a degassed organic solvent (for example DME, DMF, 1,4-dioxane, or toluene, preferably DMF) is added a palladium catalyst (for example tetrakis(triphenylphosphine)palladium(0) or bis(acetato)triphenylphosphinepalladium(II) (˜5% Pd) polymer-bound FibreCat™) (0.01-0.10 equivalents, preferably 0.05 equivalents). The reaction mixture is heated at about 50-100° C. (preferably about 80° C.) for about 2-24 hours (preferably about 18 hours) under an inert atmosphere. The reaction mixture is allowed to cool to ambient temperature and filtered. The solvents are removed under reduced pressure to afford the product that can be further purified by chromatography or crystallization.

Illustration of General Procedure JJ

### Preparation #36: 4-(3′-Cyano-biphenyl-4-yloxy)-thieno[2,3-c]pyridine-2-carboxylic acid amide

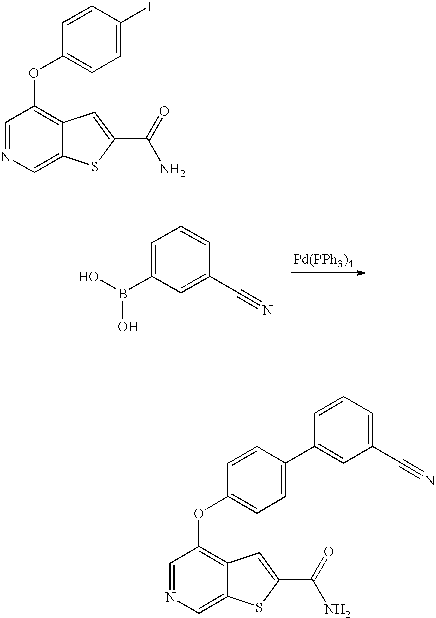

To a mixture of 4-(4-iodo-phenoxy)-thieno[2,3-c]pyridine-2-carboxylic acid amide (prepared using general procedure D) (0.050 g, 0.13 mmol), 3-cyanophenylboronic acid (0.037 g, 0.25 mmol) and 2N cesium carbonate (1.0 mL, 2.0 mmol) in degassed DME (3 mL) was added tetrakis(triphenylphosphine)palladium(0) (0.0069 g, 0.0060 mmol), at room temperature under an atmosphere of nitrogen. The reaction mixture was heated at about 80° C. for about 18 hours. The mixture was allowed to cool to ambient temperature, filtered through celite, and the solvents were removed under reduced pressure. The residue was purifed by preparative RP-HPLC (20%-100% acetonitrile/0.05M aqueous ammonium acetate, buffered to pH 4.5, over 45 min at 15 mL/min; λ=254 nm; Hypersil C18, 100 Å, 8 μm, 250×21.2 mm column) to give 4-(3′-cyano-biphenyl-4-yloxy)-thieno[2,3-c]pyridine-2-carboxylic acid amide as a light brown solid (0.032 g, 0.087 mmol); ¹H NMR (d₆-DMSO, 400 MHz): δ 9.18 (1H, s), 8.48 (1H, s), 8.22 (2H, d), 8.17 (1H, s), 8.04 (1H, d), 7.87 (1H, s), 7.84 (3H, d), 7.67 (1H, t), 7.23 (2H, d); RP-HPLC (Table 1, Method m) Rₜ 3.48 min; m/z: (M+H)⁺ 372.

Other compounds obtained using general procedure JJ are shown (Table 22).

TABLE 22
Examples synthesized using procedure JJ

| Iodide or Bromide | Boronate | | | HPLC Rₜ | |
|---|---|---|---|---|---|
| Precursor | Precursor | Product | Ex # | (Method) | m/z |
| 4-(4-Iodo-phenoxy)- | 4- | 4-(4-Pyridin-4- | 506 | 2.88 min | 348 (M + H)⁺ |
| thieno[2,3- | Pyridylboronic | yl-phenoxy)- | | (m) | |
| c]pyridine-2- | acid | thieno[2,3- | | | |
| carboxylic acid amide | | c]pyridine-2- | | | |
| (D) | | carboxylic acid | | | |
| | | amide | | | |
| 4-(4-Iodo-phenoxy)- | Thiophene-3- | 4-(4-Thiophen-3- | 507 | 3.55 min | 353 (M + H)⁺ |
| thieno[2,3- | boronic acid | yl-phenoxy)- | | (m) | |
| c]pyridine-2- | | thieno[2,3- | | | |
| carboxylic acid amide | | c]pyridine-2- | | | |
| (D) | | carboxylic acid | | | |
| | | amide | | | |
| 4-(4-Iodo-phenoxy)- | 1- | 4-(4- | 508 | 4.04 min | 403 (M + H)⁺ |
| thieno[2,3- | Benzothiophen- | Benzo[b]thiophen- | | (m) | |
| c]pyridine-2- | 3-ylboronic | 3-yl-phenoxy)- | | | |
| carboxylic acid amide | acid | thieno[2,3- | | | |
| (D) | | c]pyridine-2- | | | |
| | | carboxylic acid | | | |
| | | amide | | | |
| 4-(4-Iodo-phenoxy)- | 3,5- | 4-(3′,5′-Dichloro- | 509 | 4.33 min | 415 (M + H)⁺ |
| thieno[2,3- | Dichlorophenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 4- | 4-(4′- | 510 | 4.04 min | 415 (M + H)⁺ |
| thieno[2,3- | (Trifluoromethyl) | Trifluoromethyl- | | (m) | |
| c]pyridine-2- | phenylboronic | biphenyl-4- | | | |
| carboxylic acid amide | acid | yloxy)- | | | |
| (D) | | thieno[2,3- | | | |
| | | c]pyridine-2- | | | |
| | | carboxylic acid | | | |
| | | amide | | | |
| 4-(4-Iodo-phenoxy)- | 4- | (4-(4′-Fluoro- | 511 | 3.68 min | 365 (M + H)⁺ |
| thieno[2,3- | Fluorophenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 4- | 4-(4′-Chloro- | 512 | 3.96 min | 381 (M + H)⁺ |
| thieno[2,3- | Chlorophenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 3- | 4-(3′-Methoxy- | 513 | 3.64 min | 377 (M + H)⁺ |
| thieno[2,3- | Methoxyphenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 2- | 4-(2′-Chloro- | 514 | 3.84 min | 381 (M + H)⁺ |
| thieno[2,3- | Chlorophenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 3- | 4-(3′-Chloro- | 515 | 2.94 min | 381 (M + H)⁺ |
| thieno[2,3- | Chlorophenylboronic | biphenyl-4- | | (m) | |
| c]pyridine-2- | acid | yloxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 3,4- | 4-(4- | 516 | 2.95 min | 416 (M + H)⁺ |
| 2-(1H-tetrazol-5-yl)- | Methylenedioxyphenylboronic | Benzo[1,3]dioxol- | | (m) | |
| thieno[2,3-c]pyridine | acid | 5-yl-phenoxy)- | | | |
| (D, F, G) | | 2-(1H-tetrazol-5- | | | |
| | | yl)-thieno[2,3- | | | |
| | | c]pyridine | | | |
| 4-(4-Iodo-phenoxy)- | 1- | 4-(4-Naphthalen- | 517 | 3.33 min | 422 (M + H)⁺ |
| 2-(1H-tetrazol-5-yl)- | Naphthaleneboronic | 1-yl-phenoxy)-2- | | (m) | |
| thieno[2,3-c]pyridine | acid | (1H-tetrazol-5- | | | |
| (D, F, G) | | yl)-thieno[2,3- | | | |
| | | c]pyridine | | | |
| 4-(4-Iodo-phenoxy)- | 8- | 8-{4-[2-(1H- | 518 | 2.91 min | 423 (M + H)⁺ |
| 2-(1H-tetrazol-5-yl)- | Quinolineboronic | Tetrazol-5-yl)- | | (m) | |
| thieno[2,3-c]pyridine | acid | thieno[2,3- | | | |
| (D, F, G) | | c]pyridine-4- | | | |
| | | yloxy]-phenyl}- | | | |
| | | quinoline | | | |
| 4-(4-Iodo-phenoxy)- | 3- | 4′-[2-(1H- | 519 | 2.99 min | 430 (M + H)⁺ |
| 2-(1H-tetrazol-5-yl)- | Methoxycarbonylphenylboronic | Tetrazol-5-yl)- | | (m) | |
| thieno[2,3-c]pyridine | acid | thieno[2,3- | | | |
| (D, F, G) | | c]pyridin-4- | | | |
| | | yloxy]-biphenyl- | | | |
| | | 3-carboxylic acid | | | |
| | | methyl ester | | | |
| 4-(4-Iodo-phenoxy)- | 3- | 2-(1H-Tetrazol- | 520 | 3.33 min | 456 (M + H)⁺ |
| 2-(1H-tetrazol-5-yl)- | (Trifluoromethoxy) | 5-yl)-4-(3′- | | (m) | |
| thieno[2,3-c]pyridine | benzeneboronic | trifluoromethoxy- | | | |
| (D, F, G) | acid | biphenyl-4- | | | |
| | | yloxy)- | | | |
| | | thieno[2,3- | | | |
| | | c]pyridine | | | |
| 4-(4-Iodo-phenoxy)- | 3- | 2-(1H-Tetrazol- | 521 | 3.29 min | 440 (M + H)⁺ |
| 2-(1H-tetrazol-5-yl)- | Trifluoromethylphenylboronic | 5-yl)-4-(3′- | | (m) | |
| thieno[2,3-c]pyridine | acid | trifluoromethyl- | | | |
| (D, F, G) | | biphenyl-4- | | | |
| | | yloxy)- | | | |
| | | thieno[2,3- | | | |
| | | c]pyridine | | | |
| 4-(4-Iodo-phenoxy)- | 3- | 4-(3′-Nitro- | 522 | 3.15 min | 417 (M + H)⁺ |
| 2-(1H-tetrazol-5-yl)- | Nitrophenylboronic | biphenyl-4- | | (m) | |
| thieno[2,3-c]pyridine | acid | yloxy)-2-(1H- | | | |
| (D, F, G) | | tetrazol-5-yl)- | | | |
| | | thieno[2,3- | | | |
| | | c]pyridine | | | |
| 4-(4-Iodo-phenoxy)- | (3- | {4′-[2-(1H- | 523 | 2.86 min | 411 (M + H)⁺ |
| 2-(1H-tetrazol-5-yl)- | Cyanomethylphenyl) | Tetrazol-5-yl)- | | (m) | |
| thieno[2,3-c]pyridine | boronic | thieno[2,3- | | | |
| (D, F, G) | acid, pinacol | c]pyridin-4- | | | |
| | ester | yloxy]-biphenyl- | | | |
| | | 3-yl}-acetonitrile | | | |
| 4-(4-Iodo-phenoxy)- | 4- | 2-(1H-Tetrazol- | 524 | 3.37 min | 456 (M + H)⁺ |
| 2-(1H-tetrazol-5-yl)- | (Trifluoromethoxy) | 5-yl)-4-(4′- | | (m) | |
| thieno[2,3-c]pyridine | benzeneboronic | trifluoromethoxy- | | | |
| (D, F, G) | acid | biphenyl-4- | | | |
| | | yloxy)- | | | |
| | | thieno[2,3- | | | |
| | | c]pyridine | | | |
| 4-(4-Iodo-phenoxy)- | (4- | 4′-[2-(1H- | 525 | 2.91 min | 397 (M + H)⁺ |
| 2-(1H-tetrazol-5-yl)- | Cyanophenyl)boronic | Tetrazol-5-yl)- | | (m) | |
| thieno[2,3-c]pyridine | acid | thieno[2,3- | | | |
| (D, F, G) | | c]pyridin-4- | | | |
| | | yloxy]-biphenyl- | | | |
| | | 4-carbonitrile | | | |
| 4-(4-Iodo-phenoxy)- | 3,4- | 4-(3′,4′-Difluoro- | 526 | 3.11 min | 408 (M + H)⁺ |
| 2-(1H-tetrazol-5-yl)- | Difluorophenylboronic | biphenyl-4- | | (m) | |
| thieno[2,3-c]pyridine | acid | yloxy)-2-(1H- | | | |
| (D, F, G) | | tetrazol-5-yl)- | | | |
| | | thieno[2,3- | | | |
| | | c]pyridine | | | |
| 4-(4-Iodo-phenoxy)- | 3,4,5- | 2-(1H-Tetrazol- | 527 | 3.20 min | 426 (M + H)⁺ |
| 2-(1H-tetrazol-5-yl)- | Trifluorophenylboronic | 5-yl)-4-(3′,4′,5′- | | (m) | |
| thieno[2,3-c]pyridine | acid | trifluoro- | | | |
| (D, F, G) | | biphenyl-4- | | | |
| | | yloxy)- | | | |
| | | thieno[2,3- | | | |
| | | c]pyridine | | | |
| 4-(4-Iodo-phenoxy)- | 3,4- | 4-(3′,4′-Dichloro- | 528 | 3.39 min | 440 (M + H)⁺ |
| 2-(1H-tetrazol-5-yl)- | Dichlorophenylboronic | biphenyl-4- | | (m) | |
| thieno[2,3-c]pyridine | acid | yloxy)-2-(1H- | | | |
| (D, F, G) | | tetrazol-5-yl)- | | | |
| | | thieno[2,3- | | | |
| | | c]pyridine | | | |
| 4-(4-Iodo-phenoxy)- | (4-Fluoro-3- | 4-(4′-Fluoro-3′- | 529 | 3.19 min | 404 (M + H)⁺ |
| 2-(1H-tetrazol-5-yl)- | methylphenyl)boronic | methyl-biphenyl- | | (m) | |
| thieno[2,3-c]pyridine | acid | 4-yloxy)-2-(1H- | | | |
| (D, F, G) | | tetrazol-5-yl)- | | | |
| | | thieno[2,3- | | | |
| | | c]pyridine | | | |
| 4-(4-Iodo-phenoxy)- | (3-Fluoro-4- | 4-(3′-Fluoro-4′- | 530 | 3.00 min | 420 (M + H)⁺ |
| 2-(1H-tetrazol-5-yl)- | methoxyphenyl) | methoxy- | | (m) | |
| thieno[2,3-c]pyridine | boronic acid | biphenyl-4- | | | |
| (D, F, G) | | yloxy)-2-(1H- | | | |
| | | tetrazol-5-yl)- | | | |
| | | thieno[2,3- | | | |
| | | c]pyridine | | | |
| 4-(4-Iodo-phenoxy)- | 4-Methyl-3- | 4-(4′-Methyl-3′- | 531 | 3.31 min | 431 (M + H)⁺ |
| 2-(1H-tetrazol-5-yl)- | nitrophenylboronic | nitro-biphenyl-4- | | (m) | |
| thieno[2,3-c]pyridine | acid | yloxy)-2-(1H- | | | |
| (D, F, G) | | tetrazol-5-yl)- | | | |
| | | thieno[2,3- | | | |
| | | c]pyridine | | | |
| 4-(4-Iodo-phenoxy)- | Benzamide-3- | 4′-[2-(1H- | 532 | 2.46 min | 415 (M + H)⁺ |
| 2-(1H-tetrazol-5-yl)- | boronic acid | Tetrazol-5-yl)- | | (m) | |
| thieno[2,3-c]pyridine | | thieno[2,3- | | | |
| (D, F, G) | | c]pyridin-4- | | | |
| | | yloxy]-biphenyl- | | | |
| | | 3-carboxylic acid | | | |
| | | amide | | | |
| 4-(4-Iodo-phenoxy)- | 3-Chloro-4- | 4-(3′-Chloro-4′- | 533 | 3.29 min | 424 (M + H)⁺ |
| 2-(1H-tetrazol-5-yl)- | fluorophenylboronic | fluoro-biphenyl- | | (m) | |
| thieno[2,3-c]pyridine | acid | 4-yloxy)-2-(1H- | | | |
| (D, F, G) | | tetrazol-5-yl)- | | | |
| | | thieno[2,3- | | | |
| | | c]pyridine | | | |
| 4-(3-Bromo- | Phenylboronic | 4-(Biphenyl-3- | 534 | 3.62 min | 347 (M + H)⁺ |
| phenoxy)-thieno[2,3- | acid | yloxy)- | | (m) | |
| c]pyridine-2- | | thieno[2,3- | | | |
| carboxylic acid amide | | c]pyridine-2- | | | |
| (D) | | carboxylic acid | | | |
| | | amide | | | |
| 4-(4-Bromo- | 3,4- | 4-(4- | 535 | 3.73 min | 391 (M + H)⁺ |
| phenoxy)-thieno[2,3- | Methylenedioxyphenylboronic | Benzo[1,3]dioxol- | | (m) | |
| c]pyridine-2- | acid | 5-yl-phenoxy)- | | | |
| carboxylic acid amide | | thieno[2,3- | | | |
| (D) | | c]pyridine-2- | | | |
| | | carboxylic acid | | | |
| | | amide | | | |
| 4-(4-Iodo-phenoxy)- | 1,4- | 4-[4-(2,3- | 536 | 3.02 min | 430 (M + H)⁺ |
| 2-(1H-tetrazol-5-yl)- | Benzodioxane- | Dihydro- | | (m) | |
| thieno[2,3-c]pyridine | 6-boronic acid | benzo[1,4]dioxin- | | | |
| (D, F, G) | | 6-yl)-phenoxy]- | | | |
| | | 2-(1H-tetrazol-5- | | | |
| | | yl)-thieno[2,3- | | | |
| | | c]pyridine | | | |
| 4-(4-Iodo-phenoxy)- | 3,4- | 4-(3′,4′- | 537 | 2.49 min | 432 (M + H)⁺ |
| 2-(1H-tetrazol-5-yl)- | Dimethoxyphenylboronic | Dimethoxy- | | (m) | |
| thieno[2,3-c]pyridine | acid | biphenyl-4- | | | |
| (D, F, G) | | yloxy)-2-(1H- | | | |
| | | tetrazol-5-yl)- | | | |
| | | thieno[2,3- | | | |
| | | c]pyridine | | | |
| 4-(4-Iodo-phenoxy)- | 2,3- | 4-(2′,3′- | 538 | 2.97 min | 432 (M + H)⁺ |
| 2-(1H-tetrazol-5-yl)- | Dimethoxypheylboronic | Dimethoxy- | | (m) | |
| thieno[2,3-c]pyridine | acid | biphenyl-4- | | | |
| (D, F, G) | | yloxy)-2-(1H- | | | |
| | | tetrazol-5-yl)- | | | |
| | | thieno[2,3- | | | |
| | | c]pyridine | | | |
| 4-(4-Iodo-phenoxy)- | 3,4,5- | 2-(1H-Tetrazol- | 539 | 2.46 min | 462 (M + H)⁺ |
| 2-(1H-tetrazol-5-yl)- | Trimethoxyphenylboronic | 5-yl)-4-(3′,4′,5′- | | (m) | |
| thieno[2,3-c]pyridine | acid | trimethoxy- | | | |
| (D, F, G) | | biphenyl-4- | | | |
| | | yloxy)- | | | |
| | | thieno[2,3- | | | |
| | | c]pyridine | | | |
| 4-(4-Iodo-phenoxy)- | 2-Methoxy-4- | 3-Methoxy-4′-[2- | 540 | 2.65 min | 418 (M + H)⁺ |
| 2-(1H-tetrazol-5-yl)- | (4,4,5,5- | (1H-tetrazol-5- | | (m) | |
| thieno[2,3-c]pyridine | tetramethyl- | yl)-thieno[2,3- | | | |
| (D, F, G) | 1,3,2- | c]pyridin-4- | | | |
| | dioxaborolan- | yloxy]-biphenyl- | | | |
| | 2-yl)phenol | 4-ol | | | |
| 4-(4-Iodo-phenoxy)- | 2,5- | 4-(2′,5′- | 541 | 2.70 min | 432 (M + H)⁺ |
| 2-(1H-tetrazol-5-yl)- | Dimethoxyphenylboronic | Dimethoxy- | | (m) | |
| thieno[2,3-c]pyridine | acid | biphenyl-4- | | | |
| (D, F, G) | | yloxy)-2-(1H- | | | |
| | | tetrazol-5-yl)- | | | |
| | | thieno[2,3- | | | |
| | | c]pyridine | | | |
| 4-(4-Iodo-phenoxy)- | 4- | 4-(4′- | 542 | 2.15 min | 450 (M + H)⁺ |
| 2-(1H-tetrazol-5-yl)- | (Methanesulfonyl) | Methanesulfonyl- | | (m) | |
| thieno[2,3-c]pyridine | phenylboronic | biphenyl-4- | | | |
| (D, F, G) | acid | yloxy)-2-(1H- | | | |
| | | tetrazol-5-yl)- | | | |
| | | thieno[2,3- | | | |
| | | c]pyridine | | | |
| 4-(4-Iodo-phenoxy)- | 4- | 4-(4′- | 543 | 3.19 min | 418 (M + H)⁺ |
| 2-(1H-tetrazol-5-yl)- | (Methylthio)phenylboronic | Methylsulfanyl- | | (m) | |
| thieno[2,3-c]pyridine | acid | biphenyl-4- | | | |
| (D, F, G) | | yloxy)-2-(1H- | | | |
| | | tetrazol-5-yl)- | | | |
| | | thieno[2,3- | | | |
| | | c]pyridine | | | |

### General Procedure KK: Sonagoshira coupling of an aryl halide to an alkyne

To a mixture of an alkyne (1-5 equivalents, preferably 2 equivalents), an aryl halide (for example, an aryl bromide or an aryl iodide, preferably an aryl iodide) (preferably 1 equivalent), is added triethylamine (1-3 equivalents, preferably 2), and copper iodide (1) (0.01-0.10 equivalents, preferably 0.05 equivalents) and a degassed organic solvent (for example DME, DMF, 1,4-dioxane, or toluene, preferably DMF). To this mixture is added a palladium catalyst (for example tetrakis(triphenylphosphine)palladium(0) or bis(acetato)triphenylphosphinepalladium(II) (˜5% Pd) polymer-bound FibreCatrm) (0.01-0.10 equivalents, preferably 0.05 equivalents). The reaction mixture is heated at about 50-100° C. (preferably about 65° C.) for about 2-24 hours (preferably about 18 hours) under an inert atmosphere. The reaction mixture is allowed to cool to ambient temperature and filtered. The solvents are removed under reduced pressure to afford the product that can be further purified by chromatography or crystallization.

Illustration of General Procedure KK

### Preparation #37: 4-(4-Pyridin-3-ylethynyl-phenoxy)-thieno[2,3-c]pyridine-2-carboxylic acid amide

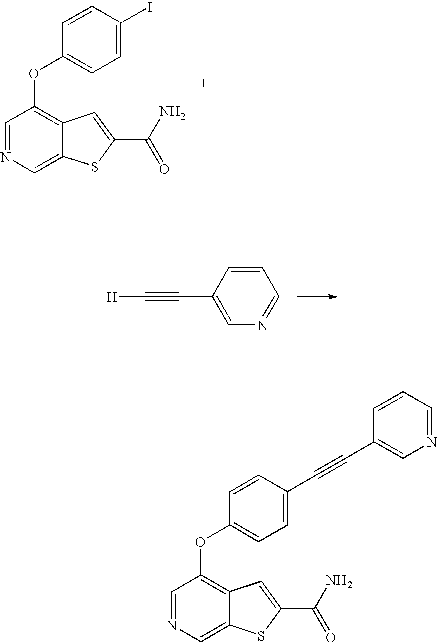

To a mixture of 4-(4-iodo-phenoxy)-thieno[2,3-c]pyridine-2-carboxylic acid amide (obtained using procedure D) (0.030 g, 0.076 mmol), 3-ethynyl-pyridine (0.011 g, 0.11 mmol), triethylamine (0.022 mL, 0.15 mmol), and copper iodide (1) (0.00072 g, 0.0038 mmol) in DMF (1.2 mL) was added tetrakis(triphenylphosphine)palladium(0) (0.0044 g, 0.0038 mmol) at room temperature. The reaction mixture was heated at 65° C. for 18 hours. The mixture was allowed to cool to ambient temperature, filtered through celite, and the solvents were removed under reduced pressure. The residue was purifed by preparative RP-HPLC (20%-100% acetonitrile/0.05M aqueous ammonium acetate, buffered to pH 4.5, over 45 min at 15 mL/min; λ=254 nm; Hypersil C18, 100 Å, 8 □m, 250×21.2 mm column) to give 4-(4-pyridin-3-ylethynyl-phenoxy)-thieno[2,3-c]pyridine-2-carboxylic acid amide as a white solid (0.013 g, 0.034 mmol); ¹H NMR (d₆-DMSO, 400 MHz): δ 9.18 (1H, s), 8.74 (1H, s), 8.57 (1H, d), 8.47 (1H, s), 8.25 (1H, s), 8.11 (1H, s), 7.95 (1H d), 7.86 (1H, s), 7.63 (2H, d), 7.47 (1H, m), 7.12 (2H, d); RP-HPLC (Table 1, Method m) Rₜ 3.40 min; m/z: (M+H)⁺ 372.

Other compounds obtained using procedure KK listed above are shown (Table 23).

TABLE 23
Examples synthesized using procedure KK

| Iodide or Bromide | | | | HPLC Rₜ | |
|---|---|---|---|---|---|
| Precursor | Alkyne | Product | Ex # | (Method) | m/z |
| 4-(4-Iodo- | Phenylacetylene | 4-(4- | 544 | 4.11 min | 371 (M + H)⁺ |
| phenoxy)- | | Phenylethynyl- | | (m) | |
| thieno[2,3- | | phenoxy)- | | | |
| c]pyridine-2- | | thieno[2,3- | | | |
| carboxylic acid | | c]pyridine-2- | | | |
| amide | | carboxylic acid | | | |
| | | amide | | | |

### General Procedure LL: Buchwald coupling of an aryl bromide with an amine.

A mixture of an aryl bromide (preferably 1 equivalent), an aliphatic or aromatic amine (1-2 equivalents, preferably 1 equivalents), an inorganic base (for example cesium carbonate or sodium tert-butoxide, preferably sodium tert-butoxide) (1-3 equivalents, preferably 1.5 equivalents), and a phosphine ligand (for example XANTPHOS, (±)-2,2′-bis(diphenylphosphino)-1,1′-binaphthalene, (R)-(+)-2,2′-bis(diphenylphosphino)-1,1′-binaphthalene, or (S)-(−)-2,2′-bis(diphenylphosphino)-1,1′-binaphthalene, preferably (+)-2,2′-bis(diphenylphosphino)-1,1′-binaphthalene) (0.01-0.2 equivalents, preferably 0.03 equivalents) is suspended in an anhydrous solvent (for example THF, toluene, 1,4-dioxane, or DMF, preferably THF) at ambient temperature under an inert atmosphere. Nitrogen gas is bubbled through the suspension for about 5-10 minutes (preferably about 5 minutes). A palladium catalyst (preferably tris(dibenzylideneacetone)dipalladium(0)) (0.002-0.2 equivalents, preferably 0.005 equivalents) is added and nitrogen gas is bubbled through the resulting suspension for about 5-10 minutes (preferably about 5 minutes). The reaction mixture is heated at about 70-1 10° C. (preferably about 80° C.) for about 1-24 hours (preferably about 12 hours). The resulting mixture is allowed to cool to ambient temperature and filtered through a celite pad. The solvent is removed in vacuo to give the product that can be further purified by crystallization or chromatography.

Illustration of General Procedure LL

### Preparation #38: 4-(4-Morpholin-4-yl-phenoxy)-2-(1H-tetrazol-5-yl)-thieno[2,3-c]pyridine

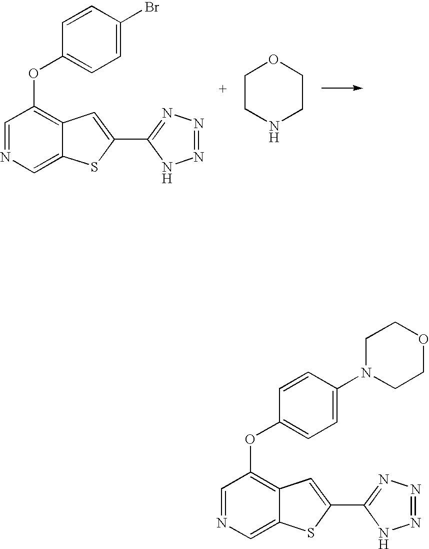

To a mixture of 4-(4-bromo-phenoxy)-2-(1H-tetrazol-5-yl)-thieno[2,3-c]pyridine (synthesized using general procedures D, F, G) (0.025 g, 0.067 mmol), morpholine (0.5 mL) and sodium tert-butoxide (0.0089 g, 0.094 mmol) in anhydrous THF (0.5 mL) were added tris(dibenzylideneacetone)dipalladium(0) (0.0003 g, 0.0003 mmol) and rac-2,2′-bis(diphenylphosphino)-1,1′-binaphthyl (0.0013 g, 0.0020 mmol), at room temperature under an atmosphere of nitrogen. The reaction mixture was heated at about 80° C. for about 18 hours. Fresh set of reagents (tris(dibenzylideneacetone)dipalladium(0), rac-2,2′-bis(diphenylphosphino)-1,1′-binaphthyl, and THF) were added, and the reaction mixture was heated again at about 80° C. for about 18 hours. The mixture was allowed to cool to ambient temperature, filtered through celite, and the solvents were removed under reduced pressure. The residue was purifed by preparative RP-HPLC (20%-100% acetonitrile/0.05M aqueous ammonium acetate, buffered to pH 4.5, over 45 min at 15 mL/min; λ=254 nm; Hypersil C18, 100 Å, 8 μm, 250×21.2 mm column) to give 4-(4-morpholin-4-yl-phenoxy)-2-(]H-tetrazol-5-yl)-thieno[2,3-c]pyridine as a white solid (0.0051 g, 0.0013 mmol); ¹H NMR (d₆-DMSO, 400 MHz): δ 9.14 (1H, s), 8.09 (1H, s), 8.02 (1H, s), 7.11 (4H, dd), 3.73 (4H, q), 3.11 (4H, q); m/z: (M+H)⁺ 381.

Other compounds obtained using the above procedure LL are shown (Table 24

TABLE 24
Examples synthesized using procedure LL

| Iodide or Bromide | | | | HPLC Rₜ | |
|---|---|---|---|---|---|
| Precursor | Amine | Product | Ex# | (Method) | m/z |
| 4-(4-Bromo-phenoxy)- | Morpholine | 4-(4-Morpholin-4-yl- | 545 | 2.45 min | 357 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid | | | |
| (D) | | | | | |
| 4-(4-Bromo-phenoxy)- | Morpholine | 4-(4-Morpholin-4-yl- | 546 | 2.80 min | 356 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid amide | | | |
| (D) | | | | | |
| 4-(4-Bromo-phenoxy)- | Aniline | 4-(4-Phenylamino- | 547 | 3.48 min | 362 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid amide | | | |
| (D) | | | | | |
| 4-(4-Bromo-phenoxy)- | Pyrrolidine | 4-(4-Pyrrolidin-1-yl- | 548 | 3.61 min | 340 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid amide | | | |
| (D) | | | | | |
| 4-(4-Bromo-phenoxy)- | 5- | 4-[4-(Indan-5- | 549 | 4.02 min | 402 (M + H)⁺ |
| thieno[2,3-c]pyridine- | Aminoindan | ylamino)-phenoxy]- | | (m) | |
| 2-carboxylic acid | | thieno[2,3-c]pyridine- | | | |
| amide | | 2-carboxylic acid | | | |
| (D) | | amide | | | |
| 4-(4-Bromo-phenoxy)- | Cyclohexyl | 4-(4-Cyclohexylamino- | 550 | 3.74 min | 368 (M + H)⁺ |
| thieno[2,3-c]pyridine- | amine | phenoxy)-thieno[2,3- | | (m) | |
| 2-carboxylic acid | | c]pyridine-2-carboxylic | | | |
| amide | | acid amide | | | |
| (D) | | | | | |
| 4-(3-Bromo-phenoxy)- | Morpholine | 4-(3-Morpholin-4-yl- | 551 | 2.49 min | 357 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid | | | |
| (D) | | | | | |
| 4-(3-Bromo-phenoxy)- | Morpholine | 4-(3-Morpholin-4-yl- | 552 | 2.95 min | 356 (M + H)⁺ |
| thieno[2,3-c]pyridine- | | phenoxy)-thieno[2,3- | | (m) | |
| 2-carboxylic acid | | c]pyridine-2-carboxylic | | | |
| amide | | acid amide | | | |
| (D) | | | | | |

### General Procedure MM: Sulfanourea formation

A mixture of an amine (preferably I equivalent) and a sulfonyl chloride (preferably 1 equivalent) is stirred in pyridine at ambient temperature for 18-120 hours (preferably 18 hours). The solvent is evaporated under reduced pressure to afford the product, which can be further purified by chromatography or crystallization.

Illustration of General Procedure MM

### Preparation #39: 4-(3-{[(Dimethylamino)sulfonyl]amino}phenyl)thieno[2,3-c]pyridine-2-carboxamide

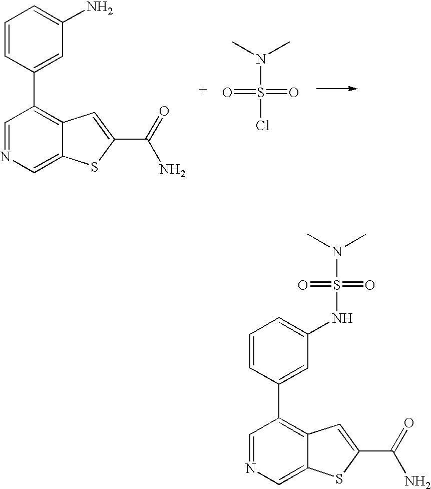

To a solution of 4-(3-amino-phenyl)-thieno[2,3-c]pyridine-2-carboxylic acid amide (0.101 g, 0.371 mmol) in pyridine (8 mL) was added dimethylsulfamoyl chloride (71 μL, 0.67 mmol) dropwise. The mixture was allowed to stir at room temperature for 5 days, and the solvents were removed under reduced pressure. The resulting product was purified by preparative RP-HPLC (20% to 80% acetonitrile/0.05M aqueous ammonium acetate, buffered to pH 4.5, over 30 min at 21 mL/min; λ=254 nm; Hypersil C18, 100 Å, 8 μm, 250×21.1 mm column) to give 4-(3-[[(dimethylamino)sulfonyl]amino)phenyl)thieno[2,3-c]pyridine-2-carboxamide (0.021 g, 0.056 mmol); RP-HPLC (table 1, method a) Rₜ 7.98 min; m/z: (M+H)⁺ 377.

Other compounds obtained using general procedure MM are shown (Table 25).

TABLE 25
Examples made using general procedure MM

| | Sulfonyl | | | HPLC | |
|---|---|---|---|---|---|
| Amine | Chloride | | | RT | |
| precursor | Precursor | Product | Ex # | (Method) | m/z |
| 4-(4-Amino- | 4,5- | 4-[4-(4,5- | 553 | 10.07 (a) | 574 |
| phenyl)thieno | Dibromothiophene- | Dibromothiophene- | | | (M + H)⁺ |
| [2,3- | 2-sulfonyl | 2-sulfonylamino)- | | | |
| c]pyridine-2- | chloride | phenyl]-thieno[2,3- | | | |
| carboxylic | | c]pyridine-2- | | | |
| acid amide | | carboxylic acid amide | | | |
| (A, C, J) | | | | | |

### Preparation #40: BOC protection of azaindole N-1 position

Preparation of 4-bromo-pyrrolo[2,3-c]pyridine-1,2-dicarboxylic acid 1-tert-butyl ester 2-methyl ester

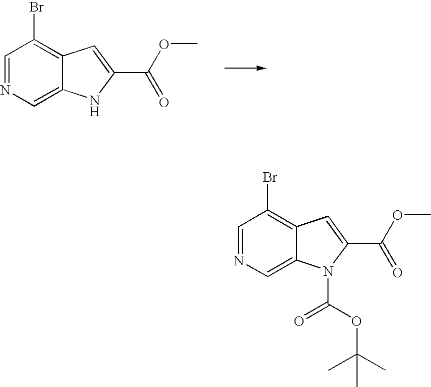

Following general procedure P, to a solution of 4-bromo-1H-pyrrolo[2,3-c]pyridine-2-carboxylic acid methyl ester (3.7 g, 14 mmol) and sodium carbonate (9.2 g, 87 mmol) in anhydrous THF (145 mL) under an inert atmosphere was added t-butyloxycarbonyl anhydride (6.6 mL, 29 mmol) dropwise. The suspension was heated at about 60° C. for about 20 hours. After filtration through a plug of Celite®, the filtrate was concentrated in vacuo and the resulting oil was diluted with EtOAc (150 mL) and saturated aqueous NaHCO₃ (70 mL). The organic portion was separated, washed with brine (3×50 mL), and dried using anhydrous sodium sulfate. Purification by silica gel chromatography using a mixture of heptane-AcOEt (7:3) as eluant gave 4-bromo-pyrrolo[2,3-c]pyridine-1,2-dicarboxylic acid 1-tert-butyl ester 2-methyl ester as a yellow solid (3.2 g, 8.7 mmol); ¹H NMR (d₆-DMSO, 400 MHz): δ 9.22 (s, 1H), 8.61 (s, 1H), 7.23 (s, 1H), 3.92 (s, 3H), and 1.60 (s, 9H). RP-HPLC (Table 1, Method n): Rₜ=4.64 min; m/z: (M+H)⁺ 356.

### General Procedure NN: Iodination of thiopyridine

To a solution of thienopyridine (preferably one equivalent) in an organic solvent (preferably DMF) is added NIS (preferably 1.1 equivalents). The resulting solution is stirred at room temperature for 12-24 hours (preferably 18 hours). Approximately half of the solvent is removed in vacuo and the resulting slurry is poured into sodium thiosulfate solution (5% in water). The resulting precipitate is collected and washed with water to afford the crude product which can be further purified by chromatography or crystallization.

Illustration of General Procedure NN

### Preparation #41: Iodination of thienopyridine core, Example 554

Preparation of 4-amino-7-biphenyl-3-yl-thieno[3,2-c]pyridine carboxylic acid

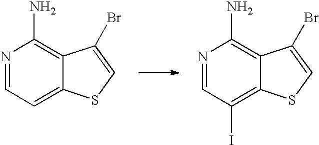

To a solution of 3-bromo-thieno[2,3-c]pyridine-4-yl-amine (1.00 g, 4.38 mmol) in DMF (15 mL) was added NIS (1.08 g, 4.81 mmol). The resulting solution was stirred at room temperature overnight. Approximately half of the solvent was removed in vacuo and the resulting slurry was poured into sodium thiosulfate (5% solution in water, 100 mL). The resulting precipitate was collected and washed with water (2×30 mL) to afford 4-amino-7-biphenyl-3-yl-thieno[3,2-c]pyridine carboxylic acid as a tan solid (1.55 g, 4.38 mmol); RP-HPLC (Table 1, Method i) Rₜ 2.72 min; m/z: (M+H)⁺ 357.1.

Preparation #42 Suzuki coupling to thienopyridine core

### Preparation of 7-biphenyl-3-yl-3-bromo-thieno[2,3-c]pyridine-4-ylamine, Example 555

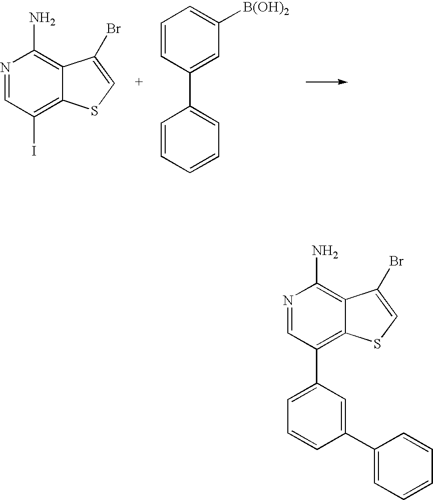

Following general procedure J, a mixture of 4-amino-7-biphenyl-3-yl-thieno[3,2-c]pyridine carboxylic acid (1.55 g, 4.38 mmol), 3-biphenylboronic acid (0.867 g, 4.38 mmol), and sodium carbonate (1.16 g, 10.9 mmol) in dioxane (43 mL) and water (20 mL) was added tetrakis(triphenylphospine)palladium(0) (0.462 g, 0.400 mmol). The resulting mixture was heated at about 80° C. for about 3 hours and then cooled to ambient temperature. The organic solvent was removed in vacuo and the resulting mixture was taken up in ethyl acetate (100 mL). The organic layer was separated and the aqueous layer was extracted with ethyl acetate (3×50 mL), washed with brine (50 mL), dried over magnesium sulfate, and concentrated in vacuo to yield 7-biphenyl-3-yl-3-bromo-thieno[2,3-c]pyridine-4-ylamine as a yellow solid (1.60 g, 4.21 mmol); RP-HPLC (Table 1, Method i) Rₜ 3.98 min; m/z: (M+H)⁺ 383.2.

### Preparation #43: Cyanation of thienopyridine core to produce 7-biphenyl-3-yl-3-cyano-thieno[2,3-c]pyridine-4-ylamine, Example 556

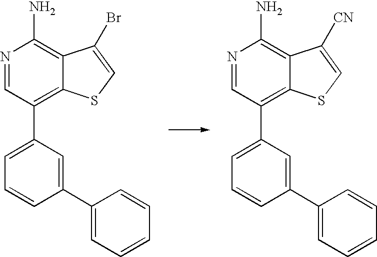

Following a procedure described in the literature (M. Alterman, A. Hallberg; J. Org. Chem. (2000), 65, 7984-89) a mixture of 7-biphenyl-3-yl-3-bromo-thieno[2,3-c]pyridine-4-ylamine (0.100 g, 0.263 mmol), zinc cyanide (0.020 g, 0.17 mmol), and tetrakis(triphenylphosphine) palladium(0) (0.009 g, 0.007 mmol) in DMF (3 mL) was heated in the microwave for 10 minutes at about 175° C. The resulting solution was purified by preparative RP-HPLC (Hypersil C18, 5 μm, 100 Å, 15 cm; 15%-85% acetonitrile −0.05 M ammonium acetate over 30 min, 21 mL/min) to yield 7-biphenyl-3-yl-3-cyano-thieno[2,3-c]pyridine-4-ylamine as a tan solid (0.035 g, 0.107 mmol); RP-HPLC (Table 1, Method i) Rₜ 3.43 min; m/z: (M+H)⁺ 328.4.

### Preparation #44: Hydrolysis of thienopyridine core to produce 4-amino-7-biphenyl-3-yl-thieno[2,3-c]pyridine-3-carboxylic acid Example 557

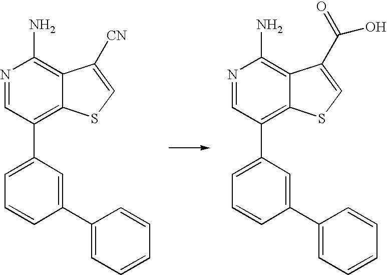

Following general procedure E, to 7-biphenyl-3-yl-3-cyano-thieno[2,3-c]pyridine-4-ylamine (0.035 g, 0.10 mmol) in ethanol (10 mL) was added sodium hydroxide pellets (0.050 g). The resulting mixture was heated at reflux for about 4 hours. The reaction was cooled to ambient temperature, the solvent removed in vacuo, and 2 N aqueous HCl (5 mL) and water (30 mL) was added. The resulting precipitate was collected by filtration to yield 4-amino-7-biphenyl-3-yl-thieno[2,3-c]pyridine-3-carboxylic acid as a tan solid (0.019 g, 0.053 mmol); RP-HPLC (Table 1, Method i) Rₜ 1.90 min; m/z: (M+H)⁺ 345.0.

### General Procedure OO: Oxidation of a nitrogen or sulfur

To a solution of a pyridine or thioether in an organic solvent (ether,methanol or dichloromethane, preferably DCM) at 0° C.-ambient temperature is added mCPBA (1-3 equivalents, preferably 1.1 equivalents). The solution is stirred at ambient temperature for about 16 hours. The solvent is removed in vacuo and the resulting solid is either washed with aqueous saturated inorganic base (preferably sodium bicarbonate) and water; or alternatively is taken up in ethyl acetate (100 mL), washed with saturated sodium bicarbonate (50 mL), brine (50 mL), and water (50 mL). The combined organic extracts are dried over dessicant (sodium or magnesium sulfate, preferably magnesium sulfate) and the solvents removed in vacuo to yield a crude mixture which can be further purified by chromatography or crystallization.

Illustration of General Procedure OO

### Preparation #45: Oxidation of sulfide to produce 4-benzenesuffonyl-thieno[2,3-c]pyridine-2-carboxylic acid amide and 4-benzenesulfinyl-thieno[2,3-c]pyridine-2-carboxylic acid amide Example 558, Example 559

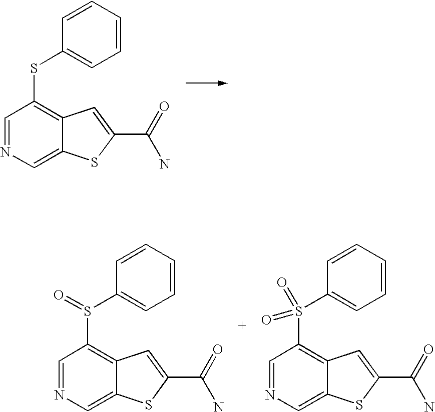

To a solution of 4-phenylsulfanyl-thieno[2,3-c]pyridine-2-carboxylic acid amide (prepared using general procedures A and D) (0.500 g, 1.74 mmol) in DCM (20 mL) was added mCPBA (1.31 g, 0.750 g). The solution was stirred at ambient temperature for about 16 hours. The solvent was removed in vacuo and the resulting solid was taken up in ethyl acetate (100 mL), washed with saturated sodium bicarbonate (50 mL), brine (50 mL), and water (50 mL). The combined organic extracts were dried over magnesium sulfate and the solvents removed in vacuo to yield a crude mixture of products which were purified by preparative RP-HPLC (Hypersil C18, 5 μm, 100 Å, 15 cm; 5%-85% acetonitrile −0.05 M ammonium acetate over 30 min, 21 mL/min) to afford 4-benzenesulfonyl-thieno[2,3-c]pyridine-2-carboxylic acid amide as a white solid (0.03 g, 0.09 mmol); RP-HPLC (Table 1, Method i) Rₜ 1.73 min; m/z: (M+H)⁺ 317.0 and 4-benzenesulfinyl-thieno[2,3-c]pyridine-2-carboxylic acid amide as a yellow powder (0.095 g, 0.314 mmol); RP-HPLC (Table 1, Method i) Rₜ 1.12 min; m/z: (M+H)⁺ 303.1.

TABLE 26
Examples made using general procedure OO

| | | | | HPLC | |
|---|---|---|---|---|---|
| | | | | RT | |
| Oxidation | | | | (Meth- | |
| Precursor | Oxidant | Product | Ex # | od) | m/z |
| 4-[4-(2- | mCPBA | 4-[4-(2- | 559A | 6.4 | 417.06 |
| Methylsulfanyl- | | Methanesulfonyl- | | min | (M − |
| ethylcarbamoyl)- | | ethylcarbamoyl)- | | (a) | H)⁻ |
| phenylamino]- | | phenylamino]- | | | |
| thieno[2,3- | | thieno[2,3- | | | |
| c]pyridine-2- | | c]pyridine-2- | | | |
| carboxylic | | carboxylic | | | |
| acid amide | | acid amide | | | |

### General procedure PP: Dehalogenation of an aryl halide

To a mixture of an aryl halide in organic solvent (ethanol, methanol or EtOAc/methanol, preferably 3:1 EtOAc/methanol) was added a catalytic amount of a palladium source (preferably palladium on carbon) under an inert atmosphere. Hydrogen is introduced to the reaction mixture and the reaction is allowed to stir at room temperature for about 6-48 hours. The catalyst is removed by filtration through a celite plug, and the solvent is removed in vacuo to yield a crude product which can be further purified by chromatography or crystallization.

Illustration of General Procedure PP

### Preparation #46 De-iodination of aryl iodide to produce 4-phenoxy-thieno[2,3-c]pyridine-2-carboxylic acid amide Example 560

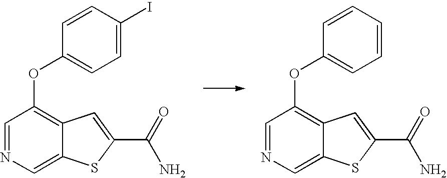

To a mixture of 4-(4-iodo-phenoxy)-thieno[2,3-c]pyridine-2-carboxylic acid amide (prepared using general procedures A and D) (0.100 g, 0.252 mmol) in EtOAc (15 mL) and methanol (5 mL) was added palladium on carbon (0.010 g) under an inert atmosphere. A balloon of hydrogen was placed over the reaction and the reaction was allowed to stir at room temperature for about 6 hours. The catalyst was removed by filtration through a celite plug, and the solvent was removed in vacuo to yield a yellow oil which was purified by preparative RP-HPLC (Hypersil C18, 5 μm, 100 Å, 15 cm; 5%-85% acetonitrile −0.05 M ammonium acetate over 30 min, 21 mL/min) to yield 4-phenoxy-thieno[2,3-c]pyridine-2-carboxylic acid as a light beige solid (0.013 g, 0.048 mmol); RP-HPLC (Table 1, Method i) Rₜ 1.94 min; m/z: (M+H)⁺ 271.3.

### Preparation #47 Suzuki coupling to azaindole to produce 4-biphenyl-3-yl-1H-pyrrolo[2,3-c]pyridine-2-carboxylic acid (Example 561)

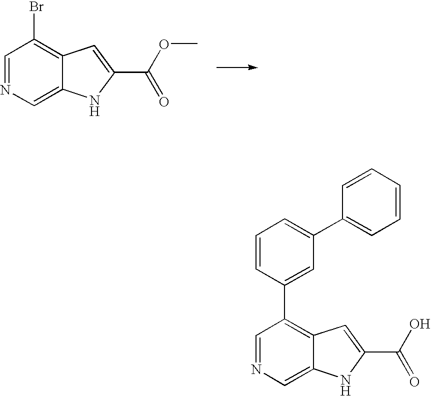

Following general procedure J, to a mixture of 4-bromo-1H-pyrrolo[2,3-c]pyridine-2-carboxylic acid methyl ester (1.20 g, 4.70 mmole), 3-biphenylboronic acid (0.978 g, 4.94 mmoles), and sodium carbonate (1.24 g, 11.7 mmol) in dioxane (30 mL) and water (15 mL) was added tetrakis(triphenylphosphine) palladium(0) (0.543 g, 0.470 mmol). The reaction mixture was heated at 80° C. for 8 hours, then cooled to ambient temperature. The dioxane was removed in vacuo. Acetic acid (2 mL) was added and the resulting solid precipitate was collected by filtration to yield 4-biphenyl-3-yl-]H-pyrrolo[2,3-c]pyridine-2-carboxylic acid: ¹H NMR (d₆-DMSO, 400 MHz): δ 8.83 (bs, 1H), 8.39 (bs, 1H), 7.93 (bs, 1H), 7.80 (m, 4H), 7.70 (m, 1H), 7.51 (m, 2H), 7.42 (m, 1H), 7.16 (bs, 1H); RP-HPLC (Hypersil C18, 5 μm, 100 Å, 15 cm; 5%-95% acetonitrile −0.05 M ammonium acetate over 15 min, 1 mL/min); Rₜ 7.55 min; m/z: (M+H)⁺ 315.2.

### General Procedure QQ: Conversion of carboxylate to ester

To a carboxylic acid (preferably 1 equivalent) was added an alkanol (preferably methanol in excess) and 1-10% by volume of sulfuric acid. The resulting mixture is heated to about reflux for 12-24 hours (preferably 18 hours), then cooled to about ambient temperature. The solvent is removed in vacuo, water (50 mL) is added, and the resulting precipitate is collected by filtration. The crude product can be further purified by crystallization or chromatography.

Illustration of General Procedure QQ

### Preparation #48: Conversion of carboxylate to methyl ester to prepare 4-biphenyl-3-yl-1H-pyrrolo[2,3-c]pyridine-2-carboxylic acid methyl ester

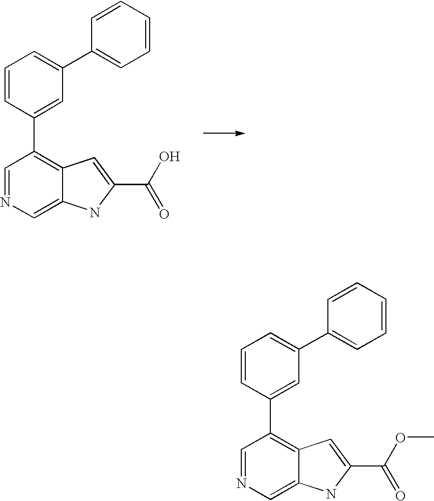

To 4-biphenyl-3-yl-1H-pyrrolo[2,3-c]pyridine-2-carboxylic acid (0.630 g, 2.00 mmol) was added methanol (20 mL) and sulfuric acid (2 mL). The resulting mixture was heated to reflux overnight, then cooled to ambient temperature. The solvent was removed in vacuo, water (50 mL) was added, and the resulting precipitate was collected by filtration to yield 4-biphenyl-3-yl-1H-pyrrolo[2,3-c]pyridine-2-carboxylic acid methyl ester as a white solid (0.160 g, 0.487 mmol); ¹H NMR (d₆-DMSO, 400 MHz): δ 9.18 (1H, s), 8.68 (1H, s), 8.08 (1H, s), 7.80-7.89 (4H, m), 7.72-7.76 (1H, m), 7.51-7.54 (3H, m), 7.41-7.45 (1H, m), 4.01 (3H, s); RP-HPLC (Hypersil C18, 5 μm, 100 Å, 15 cm; 5%-95% acetonitrile −0.05 M ammonium acetate over 15 min, 1 mL/min); Rₜ 11.64 min; mlz: (M+H)⁺ 329.0.

### Preparation #48: Dehydration of primary amide to nitrile to obtain4-biphenyl-3-yl-1H-pyrrolo[2,3-c]pyridine-2-carbonitrile, Example 562

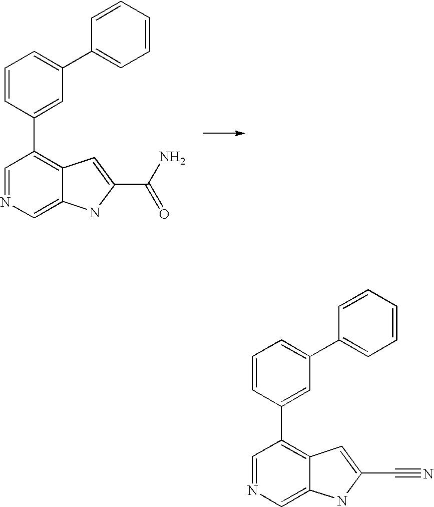

Following general procedure F, to a solution of 4-biphenyl-3-yl-1H-pyrrolo[2,3-c]pyridine-2-carboxylic acid amide (0.520 g, 1.61 mmol) in DCM (5 mL) and pyridine (1 mL) at about 0° C. was added TFAA (1 mL) dropwise. The reaction mixture was warmed to room temperature and stirred for about 2 hours. The solvent was removed in vacuo and the resulting solid was taken up in DMF (10 mL) and purified by preparative RP-HPLC (Hyperprep C18, 8 μm, 100 Å, 250 mm; 15-85% acetonitrile −0.05 M ammonium acetate over 30 min, 21 mL/min) to yield 4-biphenyl-3-yl-1H-pyrrolo[2,3-c]pyridine-2-carbonitrile: ¹H NMR (d₆-DMSO, 400 MHz) δ 8.93 (1H, s), 8.51 (1H, s), 7.94 (1H, s), 7.79-7.81 (4H, m), 7.63-7.67 (2H, m), 7.48-7.52 (2H, m), 7.41-7.48 (1H, m); RP-HPLC (Hypersil C18, 5 μm, 100 Å, 15 cm; 5%-95% acetonitrile −0.05 M ammonium acetate over 15 min, 1 mL/min); Rₜ 11.37 min; m/z: (M+H)⁺ 296.2.

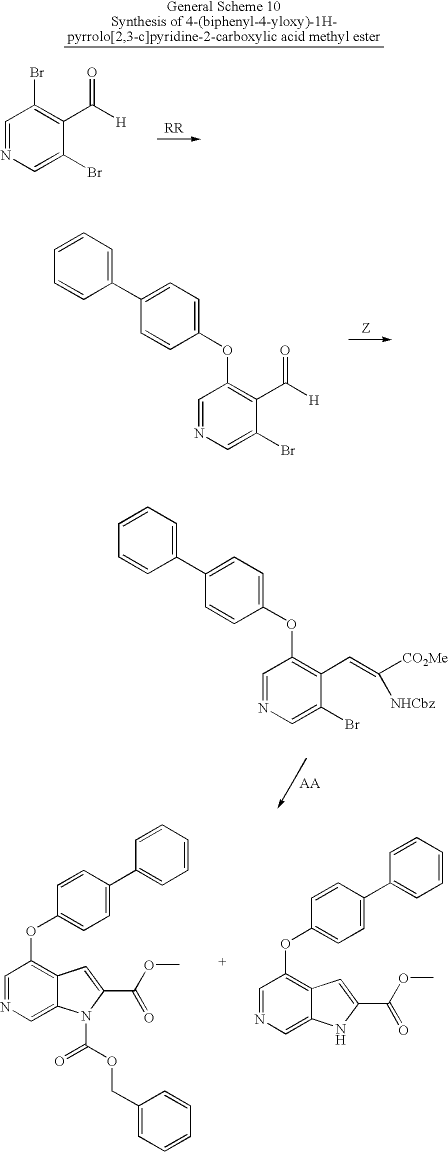

### General Procedure RR: Nucleophilic displacement of an aryl halide

To a solution of inorganic base (preferably cesium carbonate, preferably 2 equivalents) and a phenol (preferably 1 equivalent) in an organic solvent (preferably anhydrous THF) under an inert atmosphere was added a solution of an aryl halide (preferably 1-2 equivalents) in an organic solvent (preferably THF). The reaction mixture is heated at about reflux for 2-6 hours (preferably four) then is allowed to cool to room temperature. The reaction mixture is filtered and the solvent is removed in vacuo. The residue is diluted with EtOAc, washed with aqueous inorganic base (preferably sodium bicarbonate), washed with brine, and dried over dessicant (magnesium or sodium sulfate, preferably sodium sulfate) then filtered and the solvent removed in vacuo. The crude product can be further purified by chromatography or crystallization.

Illustration of General Procedure RR

### Preparation #49: Nucleophilic displacement of aryl halide to obtain 3-(Biphenyl-4-yloxy)-5-bromo-pyridine-4-carbaldehyde

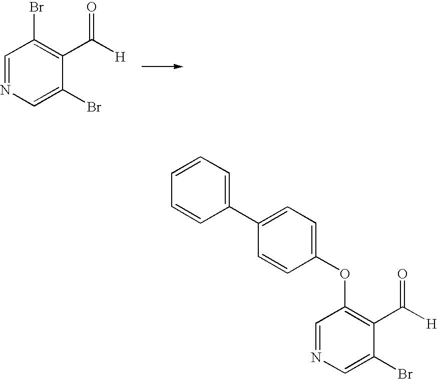

To a solution of cesium carbonate (9.20 g, 28.2 mmol) and 4-phenylphenol (2.40 g, 14.1 mmol) in anhydrous THF (100 mL) under an inert atmosphere was added a solution of 3,5-dibromopyridine-4-carboxaldehyde (7.48 g, 28.2 mmol) in THF (25 mL). The reaction mixture was heated at reflux for 4 hours then was allowed to cool to room temperature. The reaction mixture was filtered and the solvent was removed in vacuo. The residue was diluted with EtOAc, washed with aqueous sodium bicarbonate, washed with brine, and dried over sodium sulfate then filtered and the solvent removed in vacuo. Purification by silica gel (eluting with 20% EtOAc:heptane) followed by recrystallization from heptane afforded 3-(biphenyl-4-yloxy)-5-bromo-pyridine-4-carbaldehyde (4.39 g, 12.4 mmol, 87%); ¹H-NMR (DMSO-d₆, 400 MHz): δ 7.23 (m, 2H), 7.38 (m, 1H), 7.47 (m, 2H), 7.66 (m, 2H), 7.73 (m, 2H), 8.43 (s, 1H). RP-HPLC (Table 1, Method i) Rₜ=3.72 min.

### Preparation #50: Condensation of glycine ester with aryl aldehyde to obtain (Z/E)-2-Benzyloxycarbonylamino-3-[3-(biphenyl-4-yloxy)-5-bromo-pyridin-4-yl]-acrylic acid methyl ester

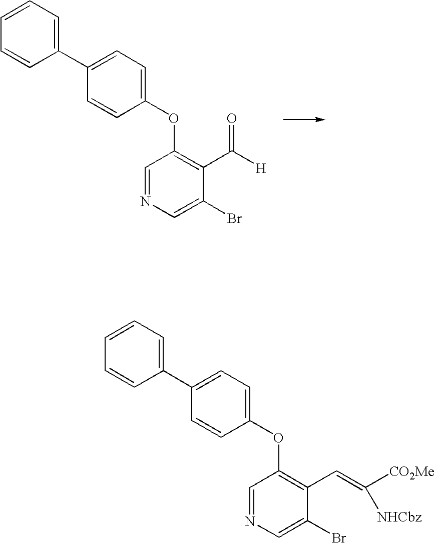

Following general procedure Z, to a solution of N-benzyloxycarbonyl-α-phosphono-glycine trimethyl ester (2.78 g, 8.40 mmol) in anhydrous DCM (50 mL) was added diazabicycloundec-7-ene (0.1M in DCM, 1.15 mL, 7.70 mmol) dropwise. The reaction mixture was stirred for about 20 minutes then a solution of 3-(biphenyl-4-yloxy)-5-bromo-pyridine-4-carbaldehyde (2.48 g, 7.00 mmol) in DCM (10 mL) was added dropwise. The resulting reaction mixture was stirred at room temperature for about 6 hours. The solvent was removed in vacuo and the residue was taken up in EtOAc (100 mL) and washed with 1N aqueous HCl (3×20 mL). The organic phase was separated, dried over sodium sulfate, and the solvent removed in vacuo. The remaining semi-solid was purified using preparative RP-HPLC. In vacuo concentration of the fractions containing the desired product provided an aqueous solution that was neutralized and extracted with EtOAc. The organic portion was separated, dried over sodium sulfate, and concentrated in vacuo to give (ZIE)-2-benzyloxycarbonylamino-3-[3-(biphenyl-4-yloxy)-5-bromo-pyridin-4-yl]-acrylic acid methyl ester (3 g, 76%) as a white solid; RP-HPLC (Table 1, Method n): Rₜ=5.40 and 5.62 min; m/z: (M+H)⁺ 345.2.

### Preparation #51: Copper mediated cyclization to azaindole to obtain 4-(biphenyl-4-yloxy)-1H-pyrrolo[2,3-c]pyridine-2-carboxylic acid methyl Example 563

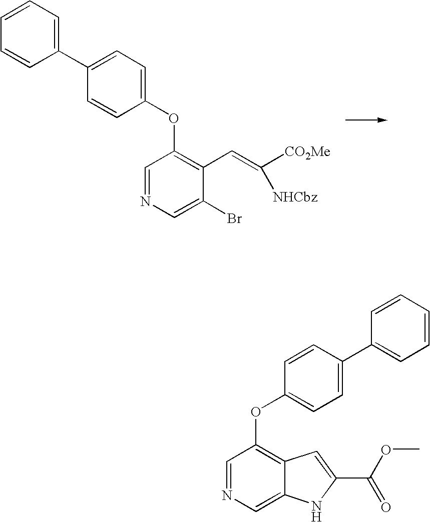

Following general procedure AA, (Z)-2-Benzyloxycarbonylamino-3-[3-(biphenyl-4-yloxy)-5-bromo-pyridin-4-yl]-acrylic acid methyl ester (2.5 g, 4.4 mmol), potassium carbonate (1.23 g, 8.94 mmol), copper (1) iodide (0.033 g, 0.17 mmol) and L-proline (0.257 g, 2.23 mmol) were added successively to an oven dried flask. Degassed 1,4-dioxane (50 mL) was added to provide a heterogenous mixture that was heated at about 90° C. for about 5 hours. The reaction mixture was cooled to ambient temperature and the solvent removed in vacuo. Water (20 mL) was added and the resulting precipitate was collected by filtration and washed with water to give 4-(biphenyl-4-yloxy)-]H-pyrrolo[2,3-c]pyridine-2-carboxylic acid methyl ester. The crude solid was used without further purification in the next step. RP-HPLC (Table 1, Method n): Rₜ=4.59 min, RP-HPLC (Table 1, Method i) Rₜ=3.17 min, m/z: (M+H)⁺ 345.1.

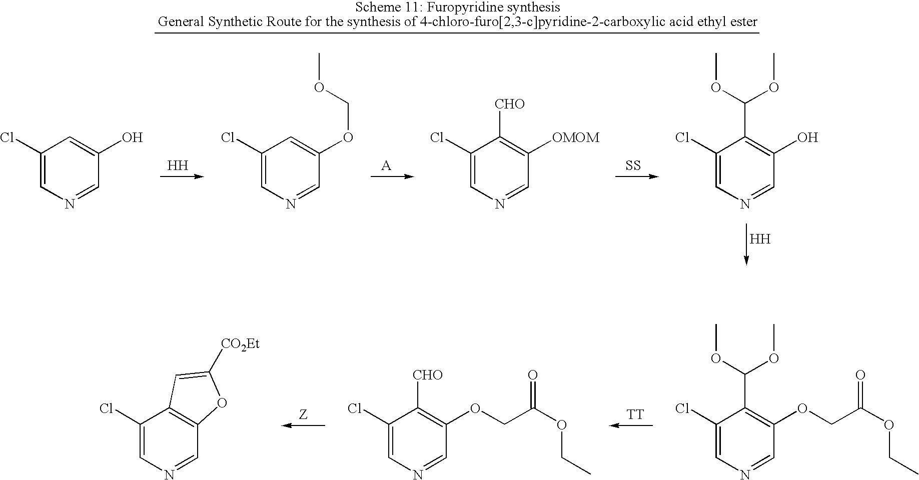

### Preparation #52: Preparation of 3-chloro-5-methoxymethoxy-pyridine

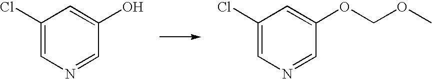

Following general procedure HH, to a 0° C. mixture of 3-chloropyridinol (3.96 g, 30.5 mmol), and diisopropylethylamine (11.7 mL, 67.2 mmol) in DCM (60 mL) was added MOMCI (2.55 mL, 33.6 mmol) dropwise. The cooling bath was removed and the solution was stirred at r.t. for about 2 hours. Saturated aqueous sodium bicarbonate solution (30 mL) was added, and the organic phase was separated, washed with saturated aqueous sodium bicarbonate solution (2×15 mL), and with brine (2×15 mL). The crude product was purified on a 5 inch silica gel plug using 9:1 AcOEt-heptane as an eluent to give 3-chloro-5-methoxymethoxy-pyridine (4.93 g, 28.3 mmol) as an oil that crystallized upon standing; ¹H-NMR (400 MHz, DMSO-d₆) δ 8.31 (d, 1H), 8.25 (d, 1H), 7.62 (d, 1H), 5.28 (s, 2H), 3.37 (s, 3H); RP-HPLC (Table 1, Method n) Rₜ 2.69 min.

### Preparation #53: Preparation of 3-chloro-5-methoxymethoxy-pyridine-4-carbaldehyde

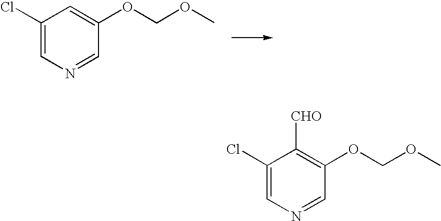

Following general procedure A, to a −78° C. solution of diisopropyl amine (1.87 mL, 13.2 mmol) in THF (40 mL) under an inert atmosphere was added n-BuLi (2.5M in hexane, 5.06 mL, 12.6 mmol). The colorless solution was stirred for about 30 minutes then a solution of 3-chloro-5-methoxymethoxy-pyridine (2.0 g, 11 mmol) in THF (0.2M) was added dropwise. The reaction mixture was stirred for about 30 minutes at −78° C., then ethyl formate (1.8 mL, 23 mmol) was added dropwise while maintaing an internal temperature below −65° C. The reaction mixture was stirred for about 3 hours at −78° C. then quenched by the addition of saturated aqueous sodium bicarbonate solution (10 mL). The mixture was allowed to warm to room temperature. The reaction mixture was extracted with EtOAc and the organic extract was concentrated in vacuo. The residue was purified using silica gel chromatography, eluting with 50% EtOAc-heptane to give 3-chloro-5-methoxymethoxy-pyridine-4-carbaldehyde (2.1g, 9.9 mmol) as a colorless oil that crystallized upon standing; ¹H-NMR (DMSO-d₆, 400 MHz) δ 10.38 (s, 1H), 8.62 (s, 1H), 8.45 (s, 1H), 5.44 (s, 2H), 3.45 (s, 3H); RP-HPLC (Table 1, Method n) Rₜ 2.46 min.

### General Procedure SS: Dimethyl acetal formation

To a solution of an aldehyde (preferably one equivalent) in an anhydrous organic alkanol (preferably methanol) is added hydrogen chloride (preferably 4.0 M in dioxane). The reaction mixture is heated at about 20-70° C. (preferably about 50° C.) for about 8-24 hours (preferably 18 hours). The reaction mixture wi cooled to ambient temperature and the solvents removed in vacuo. The residue is triturated with ethyl ether and washed with heptane. The crude product is further purified by crystallization or chromatography.

Illustration of General Procedure SS

### Preparation #54: Preparation of 5-chloro-4-dimethoxymethyl-pyridin-3-ol

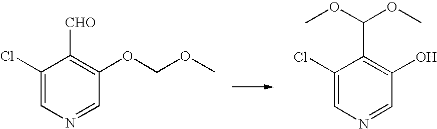

To a solution of 3-chloro-5-methoxymethoxy-pyridine-4-carbaldehyde (11.3 g, 56.2 mmol) in anhydrous methanol (100 mL) was added hydrogen chloride (4.0 M in dioxane, 8.0 mL, 32 mmol). The reaction mixture was heated at about 50° C. for about 16 h. Additional hydrogen chloride (4.0 M in dioxane, 2.0 mL, 8.0 mmol) was added and the solution heated at about 50° C. for about 24 hours. The reaction mixture was cooled to ambient temperature and the solvents removed in vacuo. The residue was triturated with ethyl ether and washed with heptane. The crude product was dissolved in 10% MeOH:DCM and excess triethylamine was added. The product was isolated by purification on a silica gel plug, using 10% MeOH:DCM as the mobile phase to afford 5-chloro-4-dimethoxymethyl-pyridin-3-ol as a pale brown solid (10.4 g, 51.2 mmol); ¹H-NMR (DMSO-d₆, 400 MHz) 8 3.38 (s, 6H), 5.69 (s, 1H), 8.06 (s, 1H), 8.15 (s, 1H), and 10.28 (s, 1H); RP-HPLC (Table 1, Method n) Rₜ 2.35 min.

### Preparation #55: Preparation of (5-chloro-4-dimethoxymethyl-pyridin-3-yloxy)-acetic acid ethyl ester

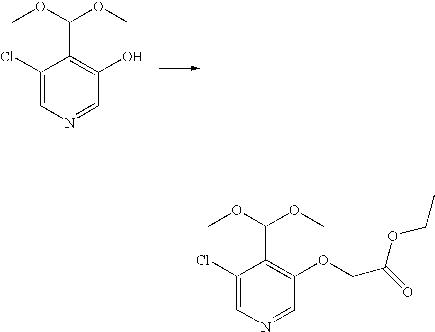

Following general procedure HH, to a solution of sodium hydride (1.60 g, 66.7 mmol) in DMF (100 mL) under an inert atmosphere was added a solution of 5-chloro-4-dimethoxymethyl-pyridin-3-ol (7.59 g, 37.3 mmol) in DMF (100 mL) dropwise at 0° C. with the aid of an additional funnel. The reaction mixture was stirred for 30 minutes then the ice bath was removed. The solution stirred for about one hour at room temperature, until no further hydrogen gas liberation was observed. A solution of ethyl bromoacetate (5.0 mL, 45 mmol) in DMF (70 mL) was added dropwise via an addition funnel and the reaction mixture was stirred for about 16 hours at ambient temperature. The reaction mixture was quenched by the addition of water (5 mL). The solvents were removed in vacuo and the residue was partitioned between ethyl acetate (200 mL) and a minimum amount of saturated aqueous bicarbonate solution (20 mL). The organic portion was separated, dried over sodium sulfate, and evaporated under reduced pressure. The residue was purified by flash column chromatography on silica gel using EtOAc:petroleum ether (50% solution) as the mobile phase to give (5-chloro-4-dimethoxymethyl-pyridin-3-yloxy)-acetic acid ethyl ester as a brown solid (7.24 g, 25.0 mmol);. ¹H-NMR (DMSO-d₆, 400 MHz) δ 1.22 (t, 3H), 3.36 (s, 6H), 4.18 (q, 2H), 5.04 (s, 2H), 5.77 (s, 1H), 8.28 (s, 1H), and 8.34 (s, 1H); RP-HPLC (Table 1, Method n) Rₜ2.90 min.

### General procedure TT: Hydrolysis of an acetal

To a solution of an acetal (preferably one equivalent) in an organic solvent (preferably THF) was added water. Trifluoroacetic acid (preferably 1.5 equivalents) was added dropwise to the stirring solution. The reaction mixture was heated at about reflux for about 8-24 hours (preferably 16 hours). The reaction mixture was cooled to about ambient temperature and concentrated in vacuo. The residue was partitioned into organic solvent (preferably ethyl acetate) and brine. The organic layer was separated, dried over a dessicant (preferably sodium sulfate), then evaporated under reduced pressure. The crude product can be further purified by chromatography or crystallization.

Illustration of General Procedure TT

### Preparation #56: Preparation of (5-chloro-4-formyl-pyridin-3-yloxy)-acetic acid ethyl ester

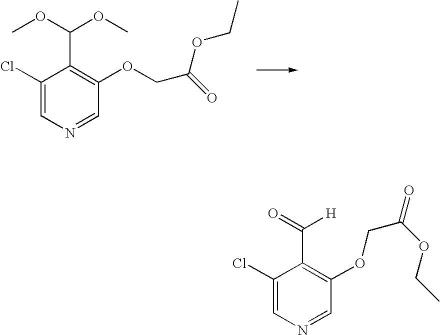

Following general procedure Z, to a solution of (5-chloro-4-dimethoxymethyl-pyridin-3-yloxy)-acetic acid ethyl ester (3.30 g, 11.4 mmol) in THF (100 mL) was added water (10 mL). Trifluoroacetic acid (1.32 mL, 17.1 mmol) was added dropwise to the stirring solution. The reaction mixture was heated at reflux for about 16 hours. The reaction mixture was cooled to ambient temperature and concentrated in vacuo. The residue was partitioned ethyl acetate (50 mL) and brine (5 mL). The organic layer was separated, dried over sodium sulfate, then evaporated under reduced pressure to afford (5-chloro-4-formyl-pyridin-3-yloxy)-acetic acid ethyl ester as a yellow solid (2.77 g, 11.4 mmol); RP-HPLC (Table 1, Method i) Rₜ 2.22 min; RP-HPLC (Table 1, Method n): Rₜ 2.81 min.

### Preparation #57: Preparation of 4-chloro-furo[2,3-c]pyridine-2-carboxylic acid ethyl ester

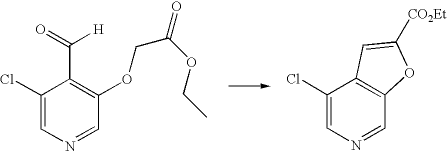

To a solution of (5-chloro-4-formyl-pyridin-3-yloxy)-acetic acid ethyl ester (0.065 g, 0.26 mmol) in toluene (5 mL) was added DBU (0.12 mL, 0.8 mmol). The reaction mixture was heated at reflux for about one hour. The reaction mixture was cooled to ambient temperature and the solvent was removed in vacuo. The residue was purified through a silica gel plug using 20% EtOAc:petroleum ether as the mobile phase and flushing with 18% MeOH:EtOAc to give 4-chloro-furo[2,3-c]pyridine-2-carboxylic acid ethyl ester as a white powder (0.036 g, 0.16 mmol); ¹H-NMR (DMSO-d₆, 400 MHz) δ 1.36 (t, 3H), 4.42 (q, 2H), 7.90 (s, 1H), 8.61 (s, 1H), 9.17 (s, 1H); RP-HPLC (Table 1, Method i) Rₜ 2.80; RP-HPLC (Table 1, Method n): Rₜ 3.57 min.

### Preparation #58: Preparation of 4-biphenyl-3-yl-furo[2,3-c]pyridine-2-carboxylic acid ethyl ester

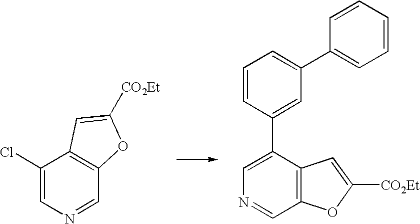

Following general procedure J, to a mixture of 4-chloro-furo[2,3-c]pyridine-2-carboxylic acid ethyl ester (0.238 g, 1.12 mmol), potassium fluoride (0.144 g, 3.7 mmol), tris(benzylideneacetone) dipalladium (0) (0.100 g, 0.11 mmol), and m-biphenyl boronic acid (0.257 g, 1.30 mmol) under inert atmosphere was added anhydrous degassed THF (5.6 mL) and PtBu₃HBF₄ (0.25 mmol). The mixture was degassed 3 times with nitrogen then heated at about 40° C. for about 16 hours. The reaction mixture was cooled to ambient temperature, diluted with EtOAc (20 mL), and filtered through a plug of celite. The filtrate was concentrated in vacuo and purified using preparative RP-HPLC to give 4-biphenyl-3-yl-furo[2,3-c]pyridine-2-carboxylic acid ethyl ester as a yellow powder (0.116 g, 0.300 mmol); RP-HPLC (Table 1, Method n) Rₜ 5.28 min; RP-HPLC (Table 1, Method i) Rₜ 3.43 min.

### Preparation #59: Preparation of 4-biphenyl-3-yl-furo[2,3-c]pyridine-2-carboxylic acid, Example 564

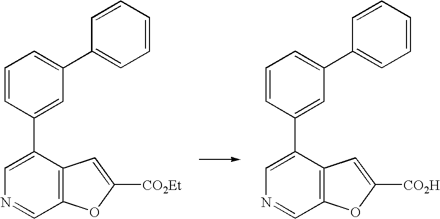

Following general procedure E, to a solution of 4-biphenyl-3-yl-furo[2,3-c]pyridine-2-carboxylic acid methyl ester (0.116 g, 0.300 mmol mmol) in MeOH (1 mL) was added 30% aqueous KOH solution (1 mL). The reaction mixture was stirred at room temperature for about 16 hours. The solvent was removed in vacuo and 3N aqueous HCl solution was added. The resulting precipitate was collected by filtration, washed with water, and dried in vacuo to give 4-biphenyl-3-yl-furo[2,3-c]pyridine-2-carboxylic acid as a white powder (0.015 g, 0.040 mmol); ¹H NMR (d₆-DMSO, 400 MHz): δ 9.30 (s, 1H), 9.29 (s, 1H), 8.0 (s, 1H), 7.87 (m, 1H), 7.82 (m, 4H), 7.80 (m, 1H), 7.53 (m, 2H), and 7.41 (m, 1H);. RP-HPLC (Table 1, Method i) Rₜ 1.52 min, m/z: (M+H)⁺ 316.2.

### General procedure UU: Addition of a nucleophile to a nitrile

A carbonitrile (preferably one equivalent), a nucleophile (hydroxylamine, hydrazine, potassium t-butoxide, the anion of trimethylsilyl diazomethane, preferably hydroxylamine) and an organic base (preferably DIEA) are combined in an organic solvent (preferably DMSO) and heated at about 20-100° C. for about 1-24 hours. The mixture is cooled to room temperature and diluted with water. The product is collected by filtration. The crude product can be further purified by crystallization or chromatography.

Illustration of General Procedure UU

### Preparation #60: Preparation of N-Hydroxy-4-(4-iodo-phenoxy)-thieno[2,3-c]pyridine-2-carboxamidine, Example 565

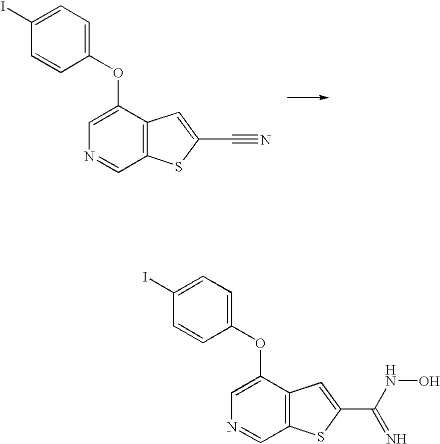

4-(4-Iodo-phenoxy)-thieno[2,3-c]pyridine-2-carbonitrile (prepared using general procedures D and F) (0.10 g, 0.27 mmol), hydroxylamine hydrochloride (0.069 g, 1.0 mmol) and DIEA (0.174 mL, 1.0 mmol) were combined in DMSO (2 mL) and heated at about 70° C. for about 1.5 hours. The mixture was cooled to room temperature and diluted with water (25 mL). The product was collected by filtration and dried in vacuo to yield N-hydroxy-4-(4-iodo-phenoxy)-thieno[2,3-c]pyridine-2-carboxamidine as an off-white solid (0.48 g, 0.25 mmol). ¹H NMR (400 MHz, DMSO-d₆) δ 6.23-6.26 (m, 2H, broad), 6.87-6.92 (m, 2H), 7.70-7.74 (m, 2H), 7.84 (s, 1H), 8.13 (s, 1H), 9.02 (s, 1H), 10.22 (s, 1H); m/z (M+H)⁺ 412.1.

### General procedure VV: Heterocycle formation

To a solution of a carboxamidine (preferably one equivalent) in an organic solvent (DMF or THF, preferably DMF), was added an electrophile (CDI, thio-CDI,trichloroacetic anhydride, trifluoroacetic acid, triethyl orthoformate in the presence of boron trifluoride, triphosgene or cyanogens bromide preferably one equivalent) and the mixture is heated at about 40-100° C. (preferably about 100° C.) for about 1-5 hours (preferably 1.5 hours). The reaction mixture is cooled to ambient temperature and the solvents removed in vacuo. The crude product can be further purified by crystallization or chromatography.

Illustration of General Procedure VV

### Preparation #61: Preparation of 3-[4-(4-Iodo-phenoxy)-thieno[2,3-c]pyridin-2-yl]-2H-[1,2,4]oxadiazol-5-one, Example 566

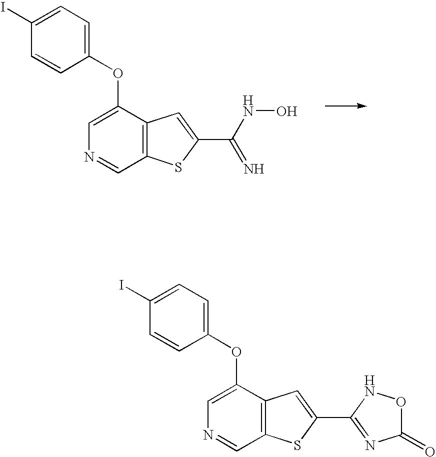

To a solution of N-hydroxy-4-(4-iodo-phenoxy)-thieno[2,3-c]pyridine-2-carboxamidine (0.062 g, 0.15 mmol) in DMF (3 mL), was added CDI (0.024 g, 0.15 mmol) and the mixture was heated at about 100° C. for about 1.5 hours. The reaction mixture was cooled to ambient temperature and the solvents removed in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 3-[4-(4-iodo-phenoxy)-thieno[2,3-c]pyridin-2-yl]-2H-[1,2,4]oxadiazol-5-one as an off white powder (0.042 g, 0.072 mmol); ¹H NMR (400 MHz, DMSO-d₆) δ 6.94-6.98 (m, 2H), 7.73-7.79 (m, 2H), 8.01 (s, 1H), 8.28 (s, 1H), 9.25 (s, 1H), 13.4 (bs, 1H); m/z: (M+H)⁺ 438.0.

### Preparation #62: 3-[4-(4-Iodo-phenoxy)-thieno[2,3-c]pyridin-2-yl]-2H-[1,2,4]thiadiazol-5-one, Example 567

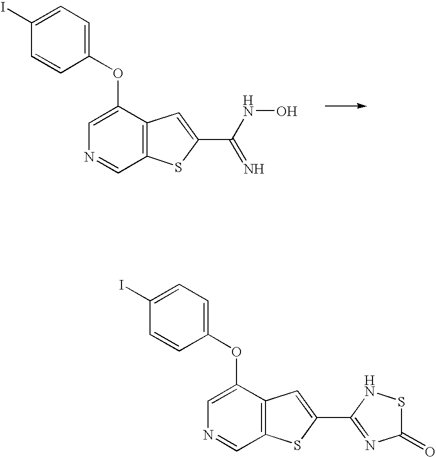

Following general procedure VV, to a solution of N-hydroxy-4-(4-iodo-phenoxy)-thieno[2,3-c]pyridine-2-carboxamidine (0.103 g, 0.250 mmol) in THF (2 mL), was added thiocarbonyldiimidazole (0.051 g, 0.26 mmol) and the mixture was stirred at room temperature under an atmosphere of nitrogen for about 1 hour. A suspension of silica gel (1.0 g) in 15% methanol/chloroform (12mL) and stirred at room temperature for about 16 hours and then heated at about 50° C. for about 2 hours. The reaction mixture was cooled to ambient temperature and the solids removed by filtration. The filtrate was concentrated in vacuo and the residue purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 3-[4-(4-iodo-phenoxy)-thieno[2,3-c]pyridin-2-yl]-2H-[1,2,4]thiadiazol-5-one as an off-white powder (0.005 g, 0.01 mmol); ¹H NMR (400 MHz, DMSO-d₆) 6.92-6.97 (m, 2H), 7.72-7.77 (m, 2H), 8.10 (s, 1H), 8.27 (s, 1H), 9.23 (s, 1H), 13.9 (bs, 1H); m/z (M−H)⁻ 452.1.

### Preparation #63: 4-(Biphenyl-4-yloxy)-N-hydroxy-thieno[2,3-c]pyridine-2-carboxamidine, Example 568

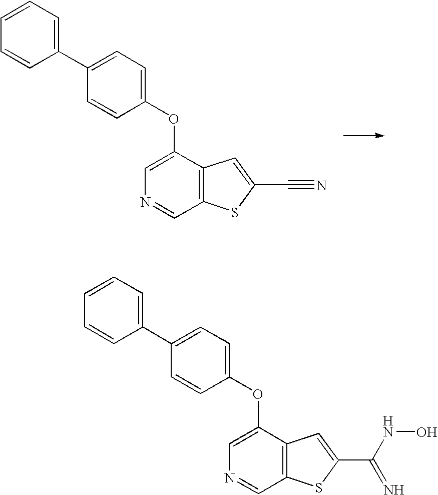

Following general procedure UU, a mixture of 4-(biphenyl-4-yloxy)-thieno[2,3-c]pyridine-2-carbonitrile (0.094 g, 0.29 mmol), hydroxylamine hydrochloride (0.069 g, 1.0 mmol) and DIEA (0.174 mL, 1.0 mmol) in DMSO (2.0 mL) was heated at about 70° C. for about 1.5 hours. The reaction mixture was cooled to room temperature and diluted with water (25 mL). The precipitate was collected by filtration and dried in vacuo to give 4-(biphenyl-4-yloxy)-N-hydroxy-thieno[2,3-c]pyridine-2-carboxamidine as a white solid (0.091 g, 0.25 mmol); RP-HPLC (Table 1, Method i) Rₜ=2.78 min; m/z: (M+H)⁺ 362.

### Preparation #64: 3-[4-(Biphenyl-4-yloxy)-thieno[2,3-c]pyridin-2-yl]-2H-[1,2,4]oxadiazol-5-one, Example 569

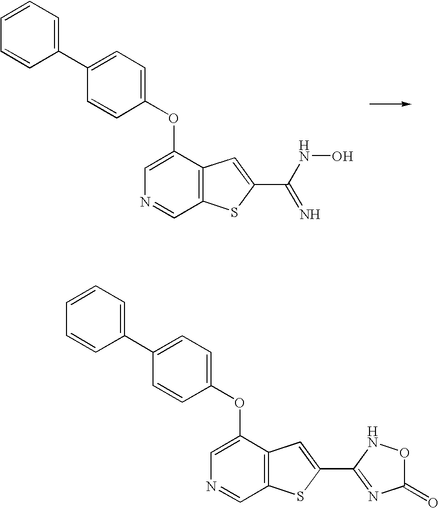

Following general procedure VV, to a solution of 4-(biphenyl-4-yloxy)-N-hydroxy-thieno[2,3-c]pyridine-2-carboxamidine (0.069 g, 0.19 mmol) in DMF (3 mL), was added CDI (0.031 g, 0.19 mmol) and the mixture was heated at about 100° C. for about 4 hours. The reaction mixture was cooled to ambient temperature and the solvents removed in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to afford 3-[4-(biphenyl-4-yloxy)-thieno[2,3-c]pyridin-yl]-2H-[1,2,4]oxadiazol-5-one as an off-white powder (0.025 g, 0.65 mmol); RP-HPLC (Table 1, Method i) Rₜ=1.88 min; m/z: (M−H)⁻ 386.

### General procedure WW: Imidazole formation

A solution of a carboxylic acid (preferably one equivalent) in in an organic solvent (preferably DMF) is treated with an inorganic base (preferably cesium carbonate, preferably one equivalent) in water (1 mL) and the reaction mixture is stirred and sonicated to yield a homogeneous mixture. The solvents are removed under reduced pressure and the residue is dissolved in an organic solvent (preferably DMF). Bromoacetophenone (preferably one equivalent) is added and the reaction mixture is stirred at room temperature for about 30 minutes. The solvents are removed under reduced pressure and ammonium acetate and an organic solvent (preferably xylenes) is added. The reaction mixture is heated at about 138° C. for about 2 hours with a Dean-Stark trap. The reaction mixture is cooled to ambient temperature and the solvents are removed under reduced pressure. The residue can be purified by chromatography or crystallization.

Illustration of General Procedure WW

### Preparation #65: 4-(4-Iodo-phenoxy)-2-(5-phenyl-1H-imidazol-2-yl)-thieno[2,3-c]pyridine, Example 570

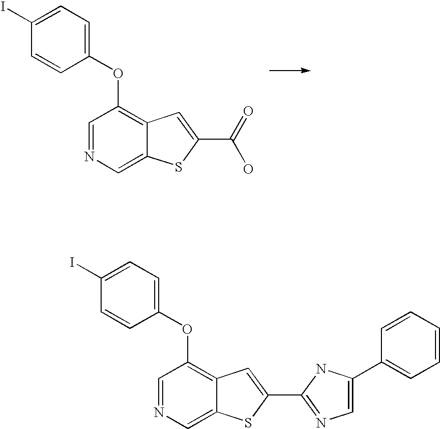

A solution of 4-(4-iodo-phenoxy)-thieno[2,3-c]pyridine-2-carboxylic acid (prepared using general procedures D and E) (0.079 g, 0.20 mmol) in DMF (1.5 mL) was treated with cesium carbonate (0.032 g, 0.10 mmol) in water (1 mL) and the reaction mixture was stirred and sonicated to yield a homogeneous mixture. The solvents were removed under reduced pressure and the residue was dissolved in DMF (2 ml). Bromoacetophenone (0.040 g, 0.20 mmol) was added and the reaction mixture was stirred at room temperature for about 30 minutes. The solvents were removed under reduced pressure and ammonium acetate (1.0 g) and xylenes (25 mL) were added. The reaction mixture was heated at about 138° C. for about 2 hours with a Dean-Stark trap. The reaction mixture was cooled to ambient temperature and the solvents were removed under reduced pressure. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column). The fractions containing product were concentrated to remove the organic solvent, neutralized by the addition of saturated aqueous sodium bicarbonate, and extracted with EtOAc (25 mL). The organic extracts were separated and the solvent was removed under reduced pressure. The residue was frozen and lyophilized to yield 4-(4-iodo-phenoxy)-2-(5-phenyl-1H-imidazol-2-yl)-thieno[2,3-c]pyridine as an off-white solid (0.020 g, 0.040 mmol); ¹H NMR (400 MHz, DMSO-d₆) 6.90-6.95 (m, 2H), 7.21-7.62 (m, 4H), 7.71-7.76 (m, 2H), 7.79-7.88 (m, 2H), 7.91 (s, 1H), 8.20 (s, 1H), 9.11 (s, 1H), 13.20 (bs, 1H); m/z: (M−H)⁻ 494.2.

### General procedure XX: Formation of hydroxymethyl imidazole

A carbonitrile (preferably one equivalent) is dissolved in an organic solvent (preferably 1,4-dioxane) containing an alkanol (preferably ethanol) then HCl gas is added as a gentle stream for about 1-2 min. The reaction mixture is stirred for about 1-4 hours at about room temperature and then the solvents are removed at reduced pressure. The residue is dissolved in 7M NH₃/MeOH and dihydroxyacetone (preferably four equivalents) is added. The mixture is heated 12-24 hours (preferably about 18 hours) in a sealed tube at about 50-100° C. (preferably 70° C.). The products are further purified by crystallization or chromatography.

Illustration of General Procedure XX

### Preparation #66: {2-[4-(Biphenyl-4-yloxy)-thieno[2,3-c]pyridin-2-yl]-3H-imidazol-4-yl}-methanol, Example 571

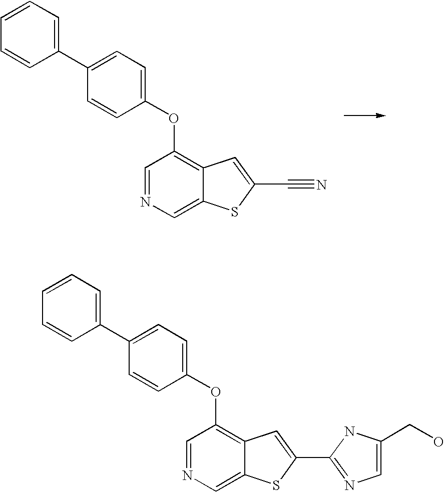

4-(Biphenyl-4-yloxy)-thieno[2,3-c]pyridine-2-carbonitrile (prepared using general procedures D and F) (0.050 g, 0.15 mmol) was dissolved in 1,4-dioxane (2.0 mL) containing ethanol (0.0089 mL) and HCl gas was added as a gentle stream for about 1 min. The reaction mixture was stirred for about 2 hours at room temperature and then the solvents were removed at reduced pressure. The residue was dissolved in 7M NH₃/MeOH (2.0 mL) and dihydroxyacetone (0.055 g, 0.61 mmol) was added. The mixture was heated overnight in a sealed tube at about 70° C. The products were purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to afford (2-[4-(Biphenyl-4-yloxy)-thieno[2,3-c]pyridin-2-yl]-3H-imidazol-4-yl}-methanol as an off white powder (0.018 g, 0.045 g); RP-HPLC (Table 1, Method i) Rₜ=1.53 min; m/z: (M+H)⁺ 400.

### General procedure YY: Heterocycle formation via the imidate

A carbonitrile (preferably one equivalent) is dissolved in an organic solvent (1,4-dioxane, preferably 1,4-dioxane) containing an alkanol (preferably ethanol) and HCl gas is added as a gentle stream for about 1-10 min (preferably one minute). The resulting solution is stirred for about 1-3 hours (preferably 2 hours) at about room temperature and then the solvents are removed in vacuo. An organic solvent (preferably dioxane) containing a nucleophile (o-phenylenediamine, ammonia, preferably one equivalent) is added and the mixture is heated at about 70-120° C. (preferably 100° C.) for about 12-24 hours (preferably 16 hours). The reaction is cooled to r.t. and the solvents removed in vacuo. The residue is further purified by chromatography or crystallization.

Illustration of General Procedure YY

### Preparation #67: 2-(1H-Benzoimidazol-2-yl)-4-(biphenyl-4-yloxy)-thieno[2,3-c]pyridine Example 572

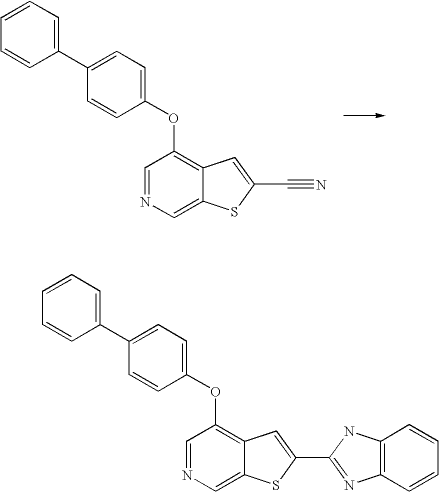

4-(Biphenyl-4-yloxy)-thieno[2,3-c]pyridine-2-carbonitrile (prepared using general procedures D and F) (0.050 g, 0.15 mmol) was dissolved in 1,4-dioxane (2.0 mL) containing ethanol (0.0089 mL) and HCl gas was added as a gentle stream for about 1 min. The resulting solution was stirred for about 2 hours at room temperature and then the solvents were removed in vacuo. Dioxane (2.0 mL) containing o-phenylenediamine (0.162 g, 1.5 mmol) was added and the mixture was heated at about 100° C. for about 16 hours. The reaction was cooled to r.t. and the solvents removed in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 2-(1H-benzoimidazol-2-yl)-4-(biphenyl-4-yloxy)-thieno[2,3-c]pyridine (0.005 g, 0.001 mmol) as an off white powder; RP-HPLC (Table 1, Method i) Rₜ=3.55 min; m/z: (M−H)⁻ 400.

### Preparation #68: 4-Biphenyl-4-ylmethyl-thieno[2,3-c]pyridine-2-carboxamidine; compound with acetic acid, Example 573

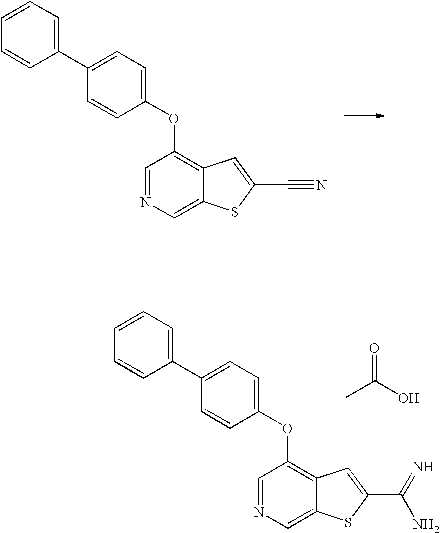

Following general procedure YY, 4-(Biphenyl-4-yloxy)-thieno[2,3-c]pyridine-2-carbonitrile (prepared using general procedures D and F) (0.050 g, 0.15 mmol) was dissolved in 1,4-dioxane (2.0 mL) containing ethanol (0.0089 mL) and HCl gas was added as a gentle stream for about 1 min. The resulting solution was stirred for about 2 hours at room temperature and then the solvents were removed in vacuo. The residue was treated with 7M NH₃/MeOH (2.0 mL) and heated at about 70° C. for about 16 hours. The products were purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 4-biphenyl-4-ylmethyl-thieno[2,3-c]pyridine-2-carboxamidine; compound with acetic acid as an off white powder (0.015 g, 0.036 mmol); RP-HPLC (Table 1, Method i) Rₜ=1.02 min; m/z: (M+H)⁺ 346.

### Preparation #69: {3-[4-(4-Iodo-phenoxy)-thieno[2,3-c]pyridin-2-yl]-ureido}-acetic acid ethyl ester, Example 574

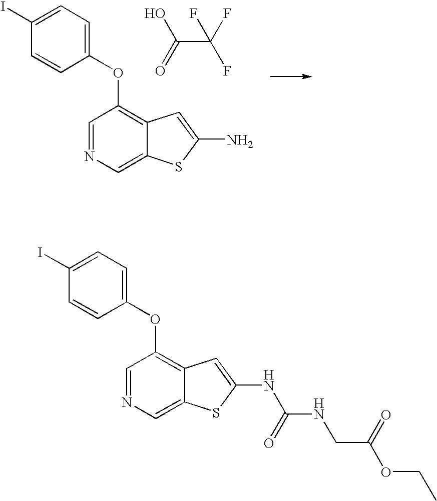

Following general procedure HH, 4-(4-Iodo-phenoxy)-thieno[2,3-c]pyridin-2-ylamine (compound with trifluoro-acetic acid) (prepared using general procedures D, E, Q, and R) (0.048 g, 0.11 mmol) was dissolved in DMF (1.0 mL) containing ethyl-diisopropyl-amine (0.065 mL, 0.37 mmol). Ethyl isocyanatoacetate (0.014 mL, 0.12 mmol) was added and the reaction mixture was stirred at about 90° C. for about 16 hours. The reaction mixture was cooled to ambient temperature and the solvents removed in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give [3-[4-(4-iodo-phenoxy)-thieno[2,3-c]pyridin-2-yl]-ureido]-acetic acid ethyl ester an off-white powder (0.025 g, 0.053 mmol); RP-HPLC (Table 1, Method i) Rₜ=3.02 min; m/z: (M+H)⁺498.

### Preparation #70: 1-[4-(4-Iodo-phenoxy)-thieno[2,3-c]pyridin-2-yl]-3-phenyl-urea, Example 575

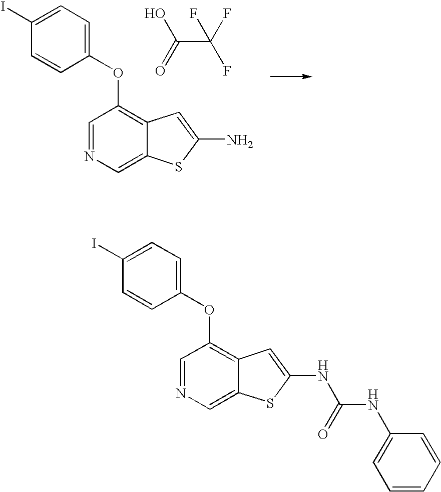

Following general procedure HH, 4-(4-Iodo-phenoxy)-thieno[2,3-c]pyridin-2-ylamine; compound with trifluoro-acetic acid (0.048 g, 0.11 mmol) was dissolved in DMF (1.0 mL) containing ethyl-diisopropyl-amine (0.065 mL, 0.37 mmol). Phenylisocyanate (0.014 mL, 0.12 mmol) was added and the reaction mixture was stirred at about 90° C. for about 16 hours. The reaction mixture was cooled to ambient temperature and concentrated at reduced pressure. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 1-[4-(4-iodo-phenoxy)-thieno[2,3-c]pyridin-2-yl]-3-phenyl-urea as an off-white powder (0.0059 g, 0.013 mmol); RP-HPLC (Table 1, Method i) Rₜ=3.62 min; m/z: (M+H)⁺ 488.

### Preparation #71: 4-Fluoro-thieno[2,3-c]pyridine-2-carboxylic acid methyl ester, Example 576

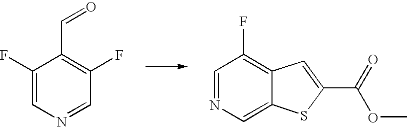

Following general procedure B, a mixture of 3,5-difluoro-pyridine-4-carbaldehyde (0.036, 0.25 mmol), methyl thioglycolate (0.022 mL, 0.25 mmol), 4A molecular sieves (0.120 g), and cesium carbonate (0.081 g, 0.25 mmol) in THF (2.0 mL) was stirred at room temperature for about 16 hours. The reaction mixture was heated at about 70° C. for about two hours. The reaction mixture was cooled to ambient temperature and concentrate under reduced pressure. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-fluoro-thieno[2,3-c]pyridine-2-carboxylic acid methyl ester as an off white powder (0.026 g, 0.012 mmol); RP-HPLC (Table 1, Method i) Rₜ=2.36 min; m/z: (M+H)⁺ 212.

### Preparation #72: 4-Fluoro-thieno[2,3-c]pyridine-2-carboxylic acid tert-butyl ester, Example 577

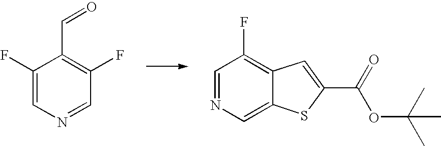

Following general procedure B, a solution of 3,5-difluoro-pyridine-4-carbaldehyde (0.029 g, 0.20 mmol), tert-butyl thioglycolate (0.020 mL, 0.20 mmol), 4 Å molecular sieves (0.10 g) and cesium carbonate (0.065 g, 0.20 mmol) in THF (2.0 mL) was stirred at room temperature for about 16 hours. The reaction mixture was heated at about 70° C. for about 16 hours, then the solvents were removed under reduced pressure. The residue was purified by RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 4-fluoro-thieno[2,3-c]pyridine-2-carboxylic acid tert-butyl ester as an off-white powder (0.015 g, 0.06 mmol); RP-HPLC (Table 1, Method i) Rₜ=3.54 min; m/z: (M+H)⁺ 254.

### General procedure ZZ: Addition of a nucleophile to a carbonyl substrate

To a solution of a carbonyl containing substrate (preferably one equivalent) and a latent nucleophile (4-bromoaniline, 1-2 equivalents preferably 1.25 equivalents) in an organic solvent (preferably THF) cooled at about −70 to −80° C. (preferably −78° C.) under an atmosphere of nitrogen is added either a base (lithium bis(trimethylsilyl)amide (1M in THF), preferably 3 equivalents relative to the nucleophile) or an organometalic reagent (alkyl Grignard or aryl Grignard, preferably one equivalent) dropwise. The reaction mixture is allowed to warm to about room temperature and saturated aqueous brine solution is added. The mixture is extracted with an organic solvent (preferably EtOAc), dried over a dessicant (preferably magnesium sulfate), filtered, and concentrated in vacuo. The residue can be further purified by chromatography or crystallization.

Illustration of General Procedure ZZ

### Preparation #73: 4-Fluoro-thieno[2,3-c]pyridine-2-carboxylic acid (4-bromophenyl)-amide Example 578

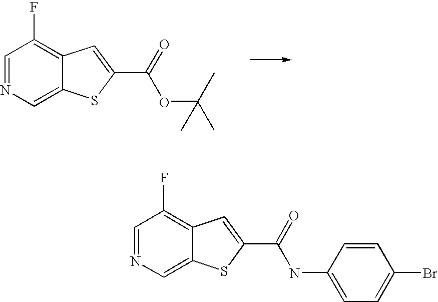

To a solution of 4-fluoro-thieno[2,3-c]pyridine-2-carboxylic acid tert-butyl ester (0.051 g, 0.20 mmol) and 4-bromoaniline (0.0043 g, 0.25 mmol) in THF (2.0 mL) cooled at about −70° C. under an atmosphere of nitrogen was added lithium bis(trimethylsilyl)amide (1M in THF, 0.75 mL, 0.75 mmol) dropwise. The reaction mixture was allowed to warm to room temperature and saturated aqueous brine solution (10 mL) was added. The mixture was extracted with EtOAc (10 mL), dried over magnesium sulfate, filtered, and concentrated in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 4-fluoro-thieno[2,3-c]pyridine-2-carboxylic acid (4-bromophenyl)-amide as an off white powder (0.014 g, 0.034 mmol); RP-HPLC (Table 1, Method i) Rₜ=3.13 min; m/z: (M−H)⁻ 349, 351.

### Preparation #74: [4-(Biphenyl-4-yloxy)-thieno[2,3-c]pyridin-2-yl]-phenyl-methanol, Example 579

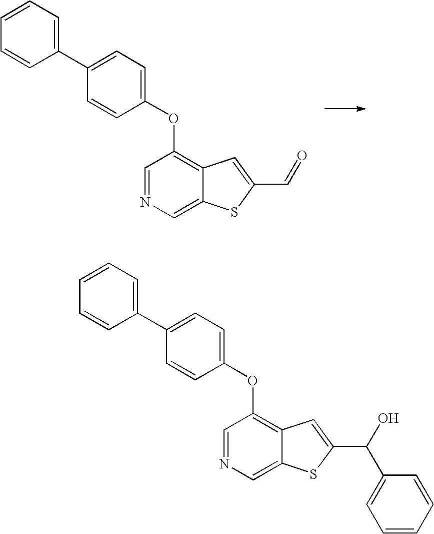

Following general procedure ZZ, aA solution of 4-(biphenyl-4-yloxy)-thieno[2,3-c]pyridine-2-carbaldehyde (0.100 g, 0.302 mmol) in THF (2.0 mL) was added dropwise to a stirred solution of phenylmagnesium chloride (2M in THF, 0.300 mL) at room temperature and the reaction mixture was stirred for about 30 minutes. Saturated aqueous brine solution (10 mL) was added and the aqueous mixture was extracted with EtOAc (3×10 mL). The organic portions were separated and combined and the solvent was removed under reduced pressure. The residue was purified in preparative RP-HPLC (20%-100% acetonitrile/0.05M aqueous ammonium acetate, buffered to pH 4.5, over 25 min at 15 muLmin; λ=254 nm; Hypersil C18, 100 Å, 8 μm, 250×21.2 mm column) to give [4-(biphenyl-4-yloxy)-thieno[2,3-c]pyridin-2-yl]-phenyl-methanol as an off-white powder (0.005 g, 0.01 mmol); RP-HPLC (Table 1, Method i) Rₜ=3.65 min; m/z: (M+H)⁺ 410.

### Preparation #76: 2-(5-Benzyloxy-pyridin-3-yl)-4-(biphenyl-4-yloxy)-thieno[2,3-c]pyridine Example 580

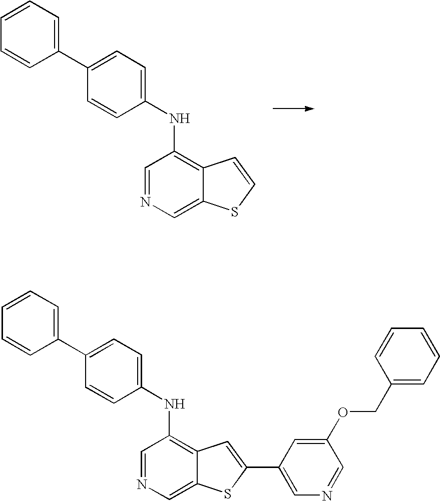

Following general procedure V, 4-(Biphenyl-4-yloxy)-thieno[2,3-c]pyridine (0.050 g, 0.16 mmol) was combined with 3-benzyloxy-5-bromo-pyridine (0.087 g, 0.30 mmol), palladium (II) acetate (0.0045 g, 0.02 mmol), biphenyl-2-yl-di-tert-butyl-phosphane (0.012 g, 0.04 mmol) and cesium carbonate (0.163 g, 0.50 mmol). The reaction mixture was purged with nitrogen and heated in a sealed vessel at about 150° C. for about 4-18 hours. The reaction mixture was cooled to ambient temperature and the solvents were removed under reduced pressure. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 2-(5-benzyloxy-pyridin-3-yl)-4-(biphenyl-4-yloxy)-thieno[2,3-c]pyridine as a yellow solid (0.0065 g, 0.013 mmol); RP-HPLC (Table 1, Method i) Rₜ=5.59 min; m/z: (M+H)⁺ 486.

### Preparation #77: 4-Bromo-6-oxy-thieno[2,3-c]pyridine-2-carboxylic acid methyl ester, Example 581

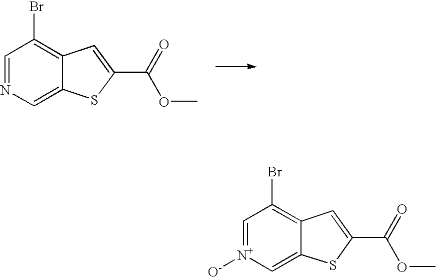

Following general procedure OO, to a stirring solution of 4-bromo-thieno[2,3-c]pyridine-2-carboxylic acid methyl ester (2.72 g, 10.0 mmol) in DCM (90 mL) was added 3-chloro-benzenecarboperoxoic acid (2.92 g, about 17.0 mmol) at room temperature. The reaction mixture was stirred for about 4 hours. The solids were removed by filtration and the filtrate was washed with saturated sodium bicarbonate solution (3×40 mL) and water (2×25 mL) and dried at about 50° C. under reduced pressure to yield 4-bromo-6-oxy-thieno[2,3-c]pyridine-2-carboxylic acid methyl ester (2.86 g, 9.90 mmol) as an off-white solid; RP-HPLC (Table 1, Method i) Rₜ=1.36 min; m/z: (M+H)⁺ 288, 290.

### General procedure AAA: Treatment of N-oxide with phosphorous oxychloride

A pyridine N-oxide (preferably one equivalent) is dissolved in phosphorus oxychloride in portions while maintaining an internal reaction temperature below about 30° C. The reaction is heated at about 20-50° C. (preferably about 40° C.) under an atmosphere of nitrogen for about 1-5 hours (preferably 2 hours), then cooled to ambient temperature and poured cautiously into either ice water or a saturated aqueous inorganic base solution (preferably sodium bicarbonate) held at about <10° C. The precipitate is collected by filtration and the crude product can be further purified by chromatography or crystallization.

Illustration of General Procedure AAA

### Preparation #78: 4-Bromo-7-chloro-thieno[2,3-c]pyridine-2-carboxylic acid methyl ester, Example 582

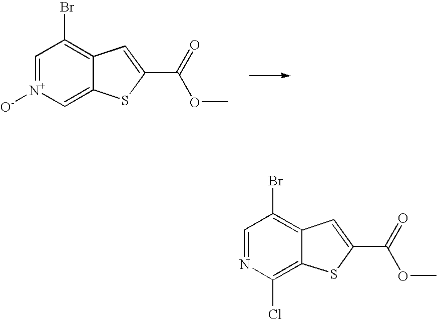

4-Bromo-6-oxy-thieno[2,3-c]pyridine-2-carboxylic acid methyl ester (1.80 g, 6.25 mmol) was dissolved in phosphorus oxychloride (18 mL) in portions while maintaining an internal reaction temperature below about 30° C. The reaction was heated at about 40° C. under an atmosphere of nitrogen for about 2 hours, then cooled to ambient temperature and poured cautiously into a saturated aqueous sodium bicarbonate solution (300 mL) held at <10° C. The precipitate was collected by filtration, dried under reduced pressure, and purified on a silica gel column using 4:1 DCM:heptane as eluant to yield 4-bromo-7-chloro-thieno[2,3-c]pyridine-2-carboxylic acid methyl ester as a white solid (1.56 g, 5.12 mmol); ¹H NMR (400 MHz, DMSO-d₆) δ 8.65 (s, 2H), 8.12 (s, 1H), 3.97 (s, 3H); RP-HPLC (Table 1, Method i) Rₜ=4.75 min. The minor isomer 4-bromo-5-chloro-thieno[2,3-c]pyridine-2-carboxylic acid methyl ester was also isolate as a pale yellow solid. ¹H NMR (400 MHz, DMSO-d₆) δ 3.96 (s, 3H), 8.04 (s, 1H), 9.26 (s, 1H); HPLC 4.11 min.

### Preparation #79: 4-(Biphenyl-4-ylamino)-7-chloro-thieno[2,3-c]pyridine-2-carboxylic acid methyl ester Example 583

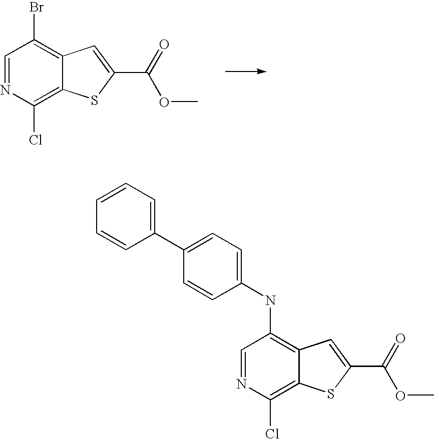

Following general procedure I, 4-Bromo-7-chloro-thieno[2,3-c]pyridine-2-carboxylic acid methyl ester (1.50 g, 4.90 mmol), Biphenyl-4-ylamine (0.911 g, 5.39 mmol), 9,9-dimethyl-4,5-bis(diphenylphosphino)xanthene (0.189 g, 0.32 mmol), Pd₂dba₃ (0.094 g, 0.16 mmol) and cesium carbonate (1.75 g, 5.39 mmol) were combined in 1,4-dioxane (25 mL). The reaction mixture was purged with nitrogen and heated at about 100° C. in a sealed vessel for about 16 hours. The reaction mixture was cooled to ambient temperature, diluted with EtOAc (50 mL), and washed with brine (25 mL). The organic portion was separated, dried over magnesium sulfate, filtered, and concentrated under reduced pressure. The residue was purified by flash chromatography on silica gel using 90% DCM:heptane as eluant to yield 4-(biphenyl-4-ylamino)-7-chloro-thieno[2,3-c]pyridine-2-carboxylic acid methyl ester as a bright yellow solid (1.03 g, 2.60 mmol); RP-HPLC (Table 1, Method i) Rₜ=5.69 min; m/z: (M+H)⁺ 395, 397.

### Preparation #80: 4-(Biphenyl-4-ylamino)-7-chloro-thieno[2,3-c]pyridine-2-carboxylic acid amide Example 584

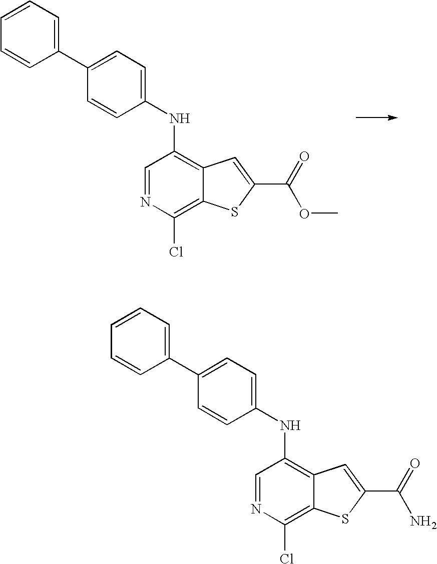

Following general procedure A mixture of 4-(biphenyl-4-ylamino)-7-chloro-thieno[2,3-c]pyridine-2-carboxylic acid methyl ester (0.025 g, 0.063 mmol) in 7M NH₃/methanol (3.0 mL) was heated in a sealed tube at about 100° C. for about 1 hour. The reaction mixture was cooled to r.t. and the solvent was partially evaporated under reduced pressure. The precipitate was collected by filtration and dried at about 50° C. under reduced pressure to provide 4-(biphenyl-4-ylamino)-7-chloro-thieno[2,3-c]pyridine-2-carboxylic acid amide as a yellow crystalline powder (0.014 g, 0.036 mmol); RP-HPLC (Table 1, Method i) Rₜ=2.74 min; m/z: (M+H)⁺ 380, 382.

### Preparation #81: 4-(Biphenyl-4-ylamino)-7-chloro-thieno[2,3-c]pyridine-2-carboxylic acid, Example 585

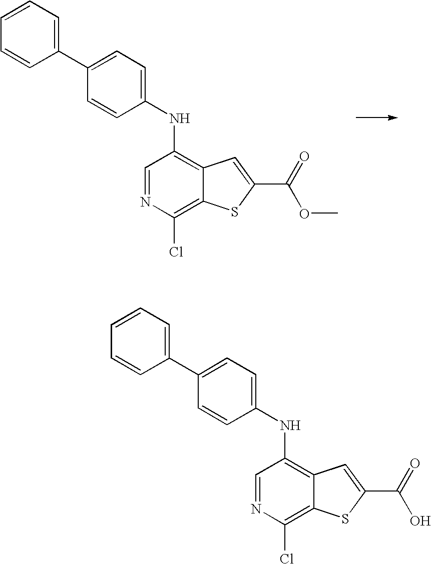

Following general procedure E, to a mixture of 4-(biphenyl-4-ylamino)-7-chloro-thieno[2,3-c]pyridine-2-carboxylic acid methyl ester (0.025 g, 0.063 mmol) in 1,4-dioxane (1.0 mL) was added 2M aqueous sodium hydroxide (0.25 mL) and the reaction mixture was heated in a sealed tube at about 100° C. for about 1 hour. The reaction mixture was cooled to ambient temperature and concentrated in vacuo. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to give 4-(biphenyl-4-ylamino)-7-chloro-thieno[2,3-c]pyridine-2-carboxylic acid (0.013 g, 0.034 mmol) as a yellow solid; RP-HPLC (Table 1, Method i) Rₜ=2.15 min; m/z: (M+H)⁺ 381, 383.

### Preparation #82: 7-Amino-4-(biphenyl-4-ylamino)-thieno[2,3-c]pyridine-2-carboxylic acid methyl ester, Example 586

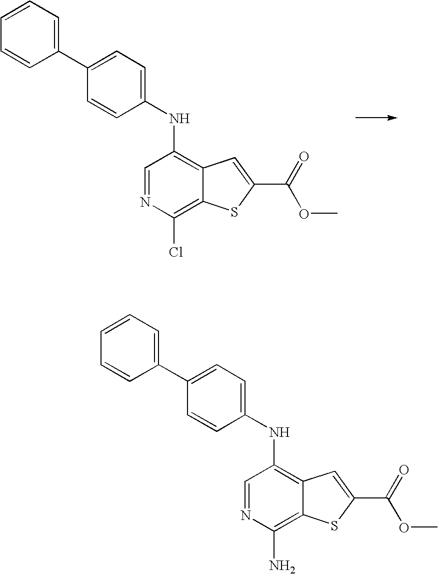

Following general procedure I, 4-(Biphenyl-4-ylamino)-7-chloro-thieno[2,3-c]pyridine-2-carboxylic acid methyl ester (0.250 g, 0.63 mmol), benzophenone imine (0.125 g, 0.69 mmol), 9,9-dimethyl-4,5-bis(diphenylphosphino)xanthene (0.029 g, 0.050 mmol), Pd₂dba₃ (0.0145 g, 0.025 mmol) and cesium carbonate (0.325 g, 1.00 mmol) were combined in 1,4-dioxane (15 mL). The mixture was purged with nitrogen and heated in a sealed tube at about 100° C. for about 16 hours. The reaction was cooled to r.t., treated with 2M HCl (1.0 mL) and stirred at room temperature for about 1 hour. The reaction mixture was diluted with EtOAc and washed with saturated aqueous sodium bicarbonate solution. The organic portion was separate, dried over magnesium sulfate, filtered, and concentrated under reduced pressure. The crude product was triturated with ether. The residue was further purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 7-amino-4-(biphenyl-4-ylamino)-thieno[2,3-c]pyridine-2-carboxylic acid methyl ester (0.014 g, 0.038 mmol) as a yellow solid; RP-HPLC (Table 1, Method i) Rₜ=3.23 min; m/z: (M+H)⁺ 376.

### Preparation #83: 7-Amino-4-(biphenyl-4-ylamino)-thieno[2,3-c]pyridine-2-carboxylic acid amide, Example 587

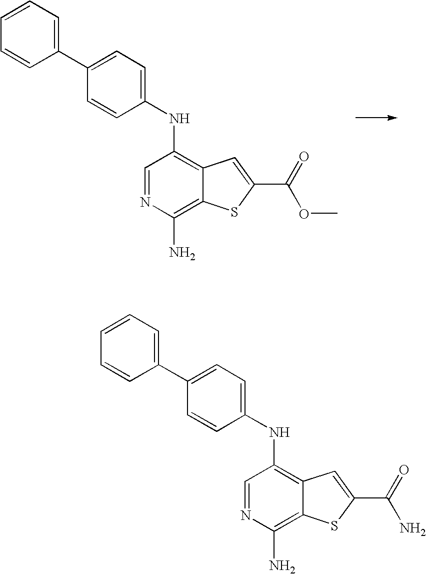

Following general procedure BB, a mixture of 7-amino-4-(biphenyl-4-ylamino)-thieno[2,3-c]pyridine-2-carboxylic acid methyl ester (0.050 g, 0.13 mmol) in 7M NH₃/methanol (3.0 mL) was heated in a sealed tube at about 100° C. for about 1 hour. The reaction mixture was cooled to r.t. and the solvent was evaporated under reduced pressure. The residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 7-amino-4-(biphenyl-4-ylamino)-thieno[2,3-c]pyridine-2-carboxylic acid amide (0.0093 g, 0.025 mmol) as a yellow solid; RP-HPLC (Table 1, Method i) Rₜ=1.77 min; m/z: (M+H)⁺ 361.

### Preparation #84: 4-(3′,5′-Dichloro-biphenyl-4-yloxy)-thieno[2,3-c]pyridine-2-carboxylic acid hydrazide, Example 588

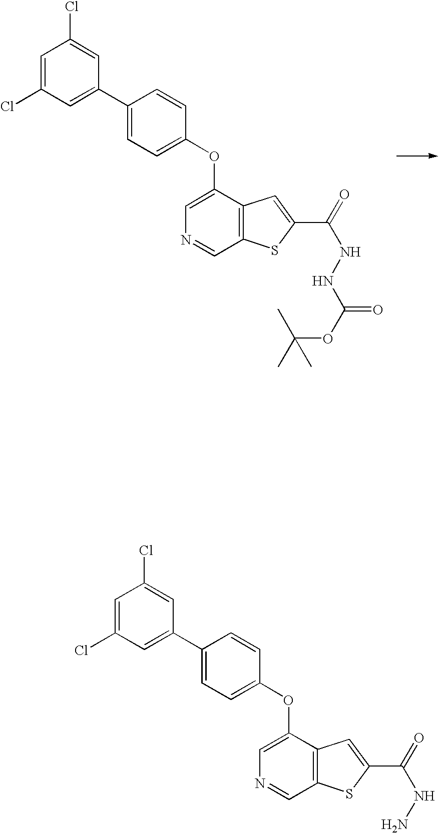

Following general procedure R, trifluoroacetic acid (2 mL, 26 mmol) was added to N′-[4-(3′,5′-dichloro-biphenyl-4-yloxy)-thieno[2,3-c]pyridine-2-carbonyl]-hydrazinecarboxylic acid tert-butyl ester (made using general procedures A, D, E, J, S) (0.044 g, 0.082 mmol) at room temperature. The solution was stirred at room temperature for about 2.5 hours. Trifluoroacetic acid was removed under reduced pressure to afford a red syrup. Diethylether (10 mL) was added to the residue to afford 4-(3′,5′-dichloro-biphenyl-4-yloxy)-thieno[2,3-c]pyridine-2-carboxylic acid hydrazide as a brown solid (0.011 g, 0.026 mmol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 11.69 min; m/z: (M−H)⁻ 428.

### Preparation #85: [4-(Biphenyl-4-ylamino)-thieno[2,3-c]pyridin-2-yl]-piperazin-1-yl-methanone Example 589

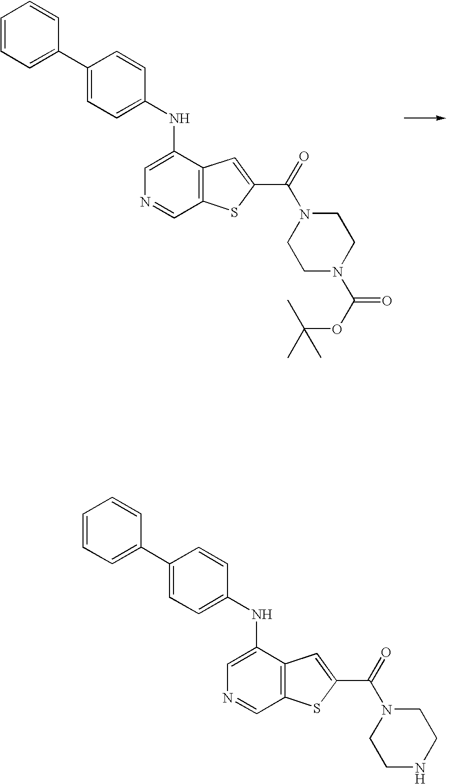

Following general procedure R, to a solution of 4-[4-(biphenyl-4-ylamino)-thieno[2,3-c]pyridine-2-carbonyl]-piperazine-1-carboxylic acid tert-butyl ester (made by general procedures A, B, I, X, S) (0.093 g, 0.18 mmol) in 1,4-dioxane (16 mL) was added 6N hydrochloric acid (4.0 mL, 24 mmol) at room temperature. The reaction mixture was stirred at room temperature for about 16 hours. The organic solvent was removed under reduced pressure and the remaining aqueous mixture was frozen and lyophilized for about 16 hours. The resulting red solid was purified by flash column chromatography on silica using 5% MeOH:DCM as a mobile phase to afford [4-biphenyl-4-ylamino)-thieno[2,3-c]pyridin-2-yl]-piperazin-1-yl-methanone as a yellow solid (0.025 g, 0.060 mmol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 8.928 min; m/z: (M+H)⁺ 415.

### Preparation #86: N-[4-(Biphenyl-4-yloxy)-thieno[2,3-c]pyridin-2-ylmethyl]-2,2,2-tri-fluoro-acetamide Example 590

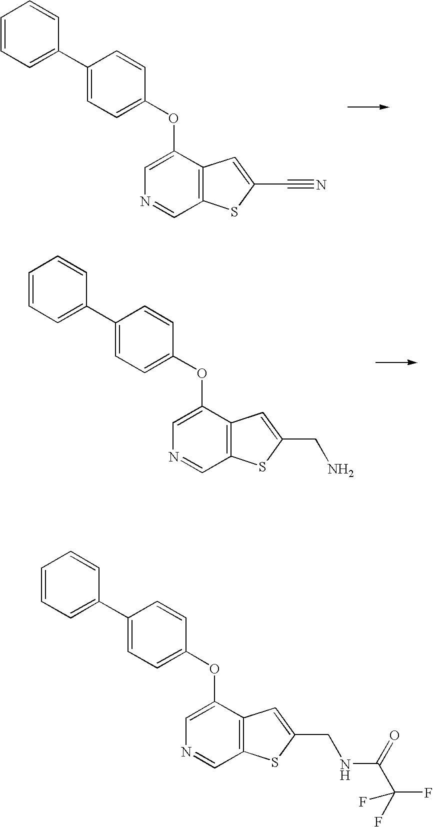

Following general procedure DD, LS-Selectride (2.2 mL, 2.2 mmol) was added to a solution of 4-biphenyl-4-yloxy)-thieno[2,3-c]pyridine-2-carbonitrile (made by general procedures A, D, F) (0.35 g, 1.1 mmol) in THF (20 mL) at about −78° C., over about 5 minutes. The solution was stirred at about −78° C. for about 1 hour, slowly allowed to warm to room temperature over about 3 hours, and stirred at room temperature for about 60 hours. The reaction was quenched by the addition of saturated aqueous ammonium chloride (50 mL). The THF was removed under reduced pressure and the remaining aqueous mixture was extracted with DCM (50 mL). The organic layer was separated and the aqueous layer was extracted further with DCM (2×50 mL). The combined organic layers were washed with brine (50 mL) and dried over anhydrous magnesium sulfate. The solvent was removed under reduced pressure and the residue was purified by flash column chromatography on silica using a gradient from 0 to 5% methanol/dichloromethane as the mobile phase to afford C-[4-biphenyl-4-yloxy)-thieno[2,3-c]pyridin-2-yl]-methylamine, Example 581, as an off-white solid (0.050 g, 0.15 mmol); m/z: (M+H)⁺ 333.

Following procedure HH, trifluoroacetic anhydride (23 μL, 0.16 mmol) was added to a solution of C-[4-biphenyl-4-yloxy)-thieno[2,3-c]pyridin-2-yl]-methylamine (0.049 g, 0.15 mmol) in anhydrous pyridine (2 mL) at about 0° C. The reaction mixture was allowed to warm to room temperature and stirred at room temperature for about 16 hours. The solvent was removed under reduced pressure and the residue purified by flash column chromatography on silica using DCM as the mobile phase. Subsequent recrystallization from diethylether and heptane afforded N-[4-(biphenyl-4-yloxy)-thieno[2,3-c]pyridin-2-ylmethyl]-2,2,2-trifluoro-acetamide as a white solid (0.012 g, 0.028 mmol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 11.932 min, m/z: (M+H)⁺ 429.

### General Procedure BBB: Preparation of an acid chloride

To a suspension of a carboxylic acid (preferably one equivalent) in an organic solvent (preferably DCM) is added oxalyl chloride (2-10 equivalents, preferably 8 equivalents) and catalytic DMF at about 0° C. The reaction mixture is allowed to warm to about room temperature and stir at about room temperature for about 6 hours. The solvents are removed under reduced pressure and the product is used immediately without purification in the next step.

Illustration of General Procedure BBB

### Preparation #87: 4-(Biphenyl-4-ylamino)-thieno[2,3-c]pyridine-2-carboxylic acid tert-butoxy-amide Example 591

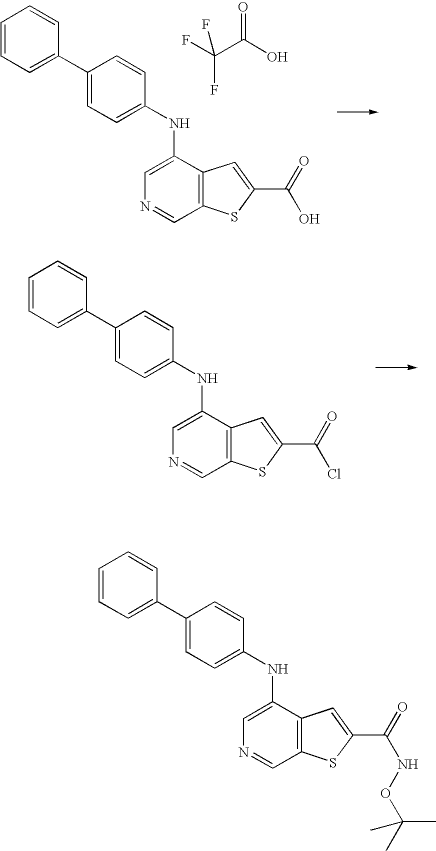

To a suspension of 4-(biphenyl-4-ylamino)-thieno[2,3-c]pyridine-2-carboxylic acid trifluoroacetate (made by general procedures A, B, I, X) (0.15 g, 0.33 mmol) in DCM (10 mL) was added oxalyl chloride (220 μL, 2.48 mmol) and catalytic DMF (5 drops) at about 0° C. The reaction mixture was allowed to warm to room temperature and stir at room temperature for about 6 hours. The solvents were removed under reduced pressure. Following general procedure HH, the residue was taken up in DMF (5 mL). To the solution of the acid chloride in DMF was added O-tert-butyl hydroxylamine hydrochloride (0.082 g, 0.65 mmol) and diisopropylethanolamine (0.180 mL, 1.01 mmol). The reaction mixture was stirred at room temperature for about 60 hours. The crude reaction mixture was purified by preparative RP-HPLC (5 to 100 % acetonitrile in 0.1 M aqueous ammonium acetate over 20 min at 21 mL/min using an 8 μ Hypersil HS C18, 250×21 mm column, λ=254 nm, Rₜ 17.7-18.0 min) to afford 4-(biphenyl-4-ylamino)-thieno[2,3-c]pyridine-2-carboxylic acid tert-butoxy-amide as a yellow solid (0.019 g, 0.046 mmol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 11.331 min; m/z: (M+H)⁺ 418.

### Preparation #88: 3-{[4-(Biphenyl-4-ylamino)-thieno[2,3-c]pyridine-2-carbonyl]-amino}-propionic acid trifluoroacetate Example 592

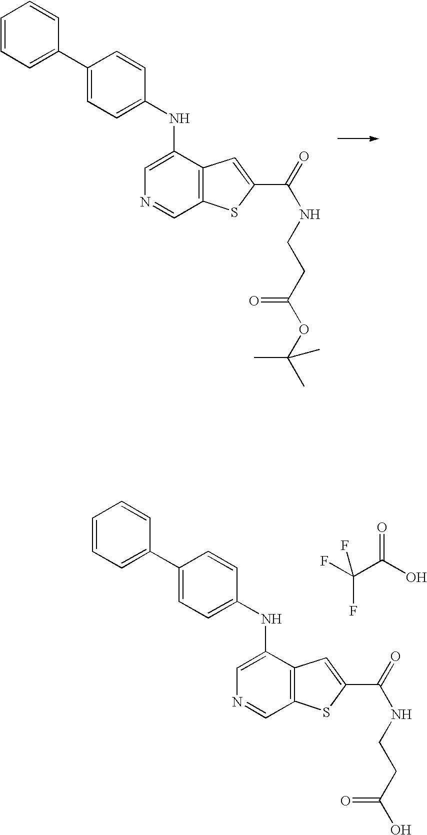

Following general procedure R, trifluoroacetic acid (4.0 mL, 52 mmol) was added to a solution of 3-{[4-(biphenyl-4-ylamino)-thieno[2,3-c]pyridine-2-carbonyl]-amino}-propionic acid tert-butyl ester (prepared using general procedures A, B, I, X, S) (0.117 g, 0.247 mmol) in DCM (6 mL) at room temperature. The reaction mixture was stirred at room temperature for about 2 hours. The solvents were removed under reduced pressure and the residue was recrystallized from ethyl acetate/heptane to afford 3-{[4-(biphenyl-4-ylamino)-thieno[2,3-c]pyridine-2-carbonyl]-amino}-propionic acid trifluoroacetate as a red solid (0.119 g, 0.224 mmol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 8.72 min; m/z: (M+H)⁺ 418.

### Preparation of #89: (R)-2-{[4-(Biphenyl-4-ylamino)-thieno[2,3-c]pyridine-2-carbonyl]-amino}-3-hydroxy-propionic acid Example 593

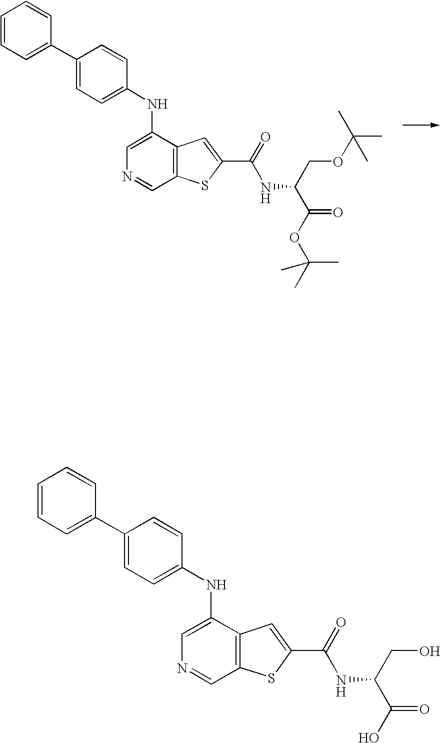

Following general procedure R, trifluoroacetic acid (2.0 mL, 26 mmol) was added to a solution of (R)-2-{[4-(biphenyl-4-ylamino)-thieno[2,3-c]pyridine-2-carbonyl]-amino}-3-tert-butoxy-propionic acid tert-butyl ester (prepared using general procedures A, B, I, X, S) (0.098 g, 0.18 mmol) in DCM (2 mL). The reaction mixture was stirred at room temperature for about 64 hours. The solvents were removed under reduced pressure and the crude residue was taken up in DMF (4 mL) and purified by preparative RP-HPLC (5 to 100 % acetonitrile in 0.1 M aqueous ammonium acetate over 20 min at 21 mL/min using an 8 μ Hypersil HS C18, 250×21 mm column, λ=254 nm, Rₜ 10.8-12.1 min) to afford (R)-2-([4-(biphenyl-4-ylamino)-thieno[2,3-c]pyridine-2-carbonyl]-amino}-3-hydroxy-propionic acid as a yellow solid (0.046 g, 0.11 mmol); RP-HPLC (5% to 100% acetonitrile/0.05M aqueous ammonium acetate, buffered to pH 4.5, over 10 min at 1.0 mL/min; λ=254 nm; Hypersil C18, 100 Å, 5 μm, 250×4.6 mm column) Rₜ 7.975 min; m/z: (M+H)⁺ 434.

### Preparation of #90: [4-(Biphenyl-4-ylamino)-thieno[2,3-c]pyridin-2-yl]-methanol Example 594

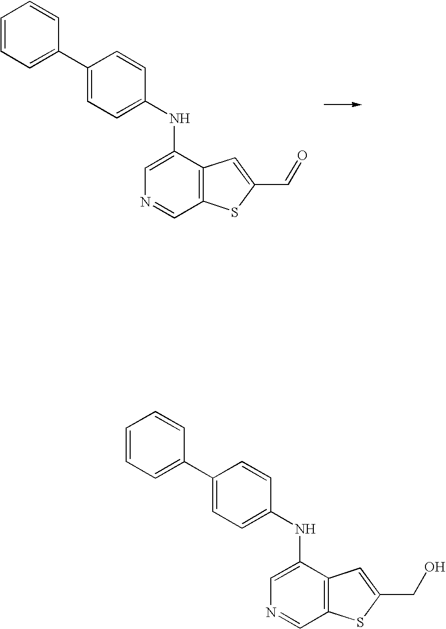

Following general procedure DD, sodium borohydride (0.0057 g, 0.15 mmol) was added to a suspension of 4-biphenyl-4-ylamino)-thieno[2,3-c]pyridine-2-carbaldehyde (0.050 g, 0.15 mmol) in methanol (2 mL) at about 0° C. The reaction mixture was allowed to warm to room temperature and was stirred at room temperature for about 1 hour. Water (2 mL) was added and the organic solvent was removed under reduced pressure. Hydrochloric acid (1N aqueous solution, 10 mL) was added to the aqueous phase and to it was added DCM (10 mL). A suspension resulted. The aqueous layer was basified to pH 9 with 10% aqueous sodium hydroxide and the organic layer was separated. The aqueous layer was extracted twice more with DCM (2×10 mL). The combined organic layers were washed with brine (20 mL) and dried over magnesium sulfate. The solvent was removed under reduced pressure. Diethyl ether (4 mL) was added to the residue and the solid was filtered off to afford [4-(biphenyl-4-ylamino)-thieno[2,3-c]pyridin-2-yl]-methanol as an orange solid (0.024 g, 0.072 mmol); RP-HPLC (5% to 100% acetonitrile/0.05M aqueous ammonium acetate, buffered to pH 4.5, over 10 min at 1.0 mL/min; λ=254 nm; Hypersil C18, 100 Å, 5 μm, 250×4.6 mm column) Rₜ10.553 min, m/z: (M+H)⁺ 333.

### Preparation of #91: 4-(Biphenyl-4-ylamino)-thieno[2,3-c]pyridine-2-carbaldehyde

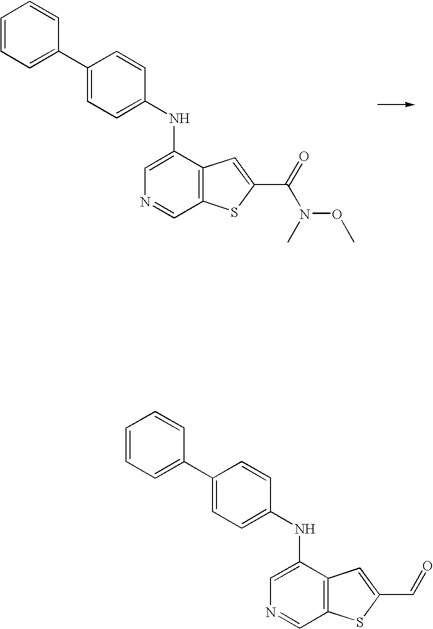

Following general procedure DD, lithium aluminium hydride (1 M solution in tetrahydrofuran, 4 mL, 4 mmol) was added to a solution of 4-biphenyl-4-ylamino)-thieno[2,3-c]pyridine-2-carboxylic acid methoxy-methyl-amide (made by general procedures A, B, I, X, S) (0.78 g, 2.0 mmol) over 10 minutes, at −78° C. The reaction mixture remained stirring at −78° C. for approximately 45 minutes. The cooling bath was removed and sodium sulfate decahydrate was added to the reaction mixture until it turned clear. The salts were removed by filtration and the filtrate was concentrated in vacuo to afford an orange residue. Diethyl ether (5 mL) was added to the residue the product was collected by filtration to afford 4-(biphenyl-4-ylamino)-thieno[2,3-c]pyridine-2-carbaldehyde as an orange solid (0.44 g, 1.33 mmol); RP-HPLC (5% to 100% acetonitrile/0.05M aqueous ammonium acetate, buffered to pH 4.5, over 10 min at 1.0 mL/min; λ=254 nm; Hypersil C18, 100 Å, 5 μm, 250×4.6 mm column) R, 12.35 min, m/z: (M+H)⁺ 331.

### Preparation #92: 4-[(4-Bromo-phenyl)-methyl-amino]-thieno[2,3-c]pyridine-2-carboxylic acid amide Example 595

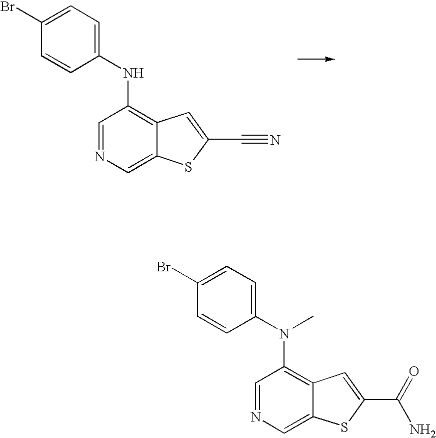

Following general procedure HH, to a solution of 4-(4-bromo-phenylamino)-thieno[2,3-c]pyridine-2-carbonitrile (0.060 g, 0.18 mmol) in THF (1 mL) at about −78° C. was added a solution of potassium t-butoxide (0.025 g, 0.22 mmol) in THF (0.5 mL). To the resulting dark purple solution was added methyl iodide (0.014 mL, 0.22 mmol). The reaction mixture was allowed to warm to r.t. over about 10 minutes. Following general procedure H, water (1 mL) was added and the reaction mixture was heated at about 100° C. for about 40 h. After cooling to r.t. the crude reaction mixture was diluted with DMF (5 mL), filtered, and purified by preparative RP-HPLC (20% to 50% acetonitrile/0.05M aqueous ammonium acetate, buffered to pH 4.5, over 25 min, then 60% to 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to provide 4-[(4-bromo-phenyl)-methyl-amino]-thieno[2,3-c]pyridine-2-carboxylic acid amide (0.30 g, 0.083 mmol) as a yellow solid; RP-HPLC (Table 1, Method a) 9.52 min; m/z: (M+H)⁺ 362, 364.

### Preparation #93: 4-Chloro-benzo[b]thiophene-2-carboxylic acid amide, Example 596

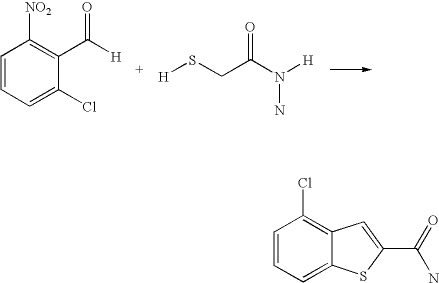

Following general procedure C, to a mixture of 2-chloro-6-nitrobenzaldehyde (0.100 g, 0.538 mmol) and cesium carbonate (0.175 g, 0.538 mmol) in THF (25 mL) was added thioacetamide (0.490 mL, 0.538 mmol). The resulting mixture was heated at about 60° C. for about 2 hours. The solvent was removed in vacuo and the residue taken up in DMF (8 mL). The crude solution was purified by preparative RP-HPLC (Hypersil C18, 5 μm, 100 Å, 15 cm; 15%-85% acetonitrile −0.05 M ammonium acetate over 30 min, 21 mL/min) to yield 4-chloro-benzo[b]thiophene-2-carboxylic acid amide as a light yellow soild (0.049 g, 0.23 mmol); RP-HPLC (Table 1, Method i) Rₜ 2.17 min; m/z: (M+H)⁻ 210.3.

### Preparation #94: 4-(4′-Trifluoromethyl-biphenyl-4-yl-oxy)-thieno[2,3-c]pyridine, Example 597

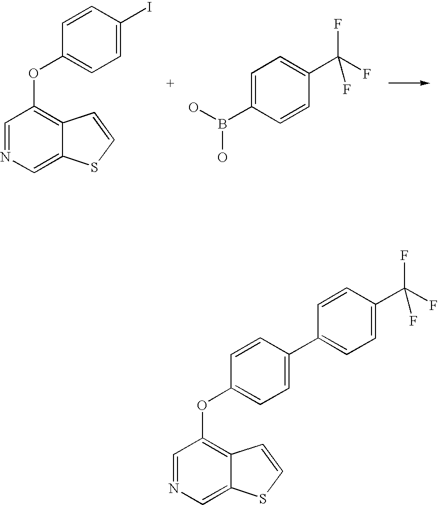

Following general procedure J, to a mixture of 4-(4-iodo-phenoxy)-thieno[2,3-c]pyridine (0.124 g, 0.351 mmol), 4-trifluoromethylbenzene boronic acid (0.066 g, 0.35 mmol), sodium carbonate (0.093 g, 0.88 mmol) in DME (10 mL) and water (3 mL) was added tetrakis(triphenylphosphine) palladium (0) (0.034 g, 0.030 mmol). The resulting mixture was heated at about 80° C. for about 3 hours and then cooled to ambient temperature. The organic solvent was removed in vacuo and the resulting mixture was taken up in ethyl acetate (50 mL). The organic layer was separated and the aqueous layer was extracted with ethyl acetate (3×30 mL), washed with brine (30 mL), dried over magnesium sulfate, and concentrated in vacuo to yield 4-(4′-trifluoromethyl-biphenyl-4-yl-oxy)-thieno[2,3-c]pyridine(0.053 g, 0.14 mmol); RP-HPLC (Table 1, Method i) Rₜ 13.56 min; m/z: (M+H)⁺ 372.2.

### Preparation #95: 4,7-Bis-biphenyl-3-yl-thieno[2,3-c]pyridine-2-carboxylic acid Example 598

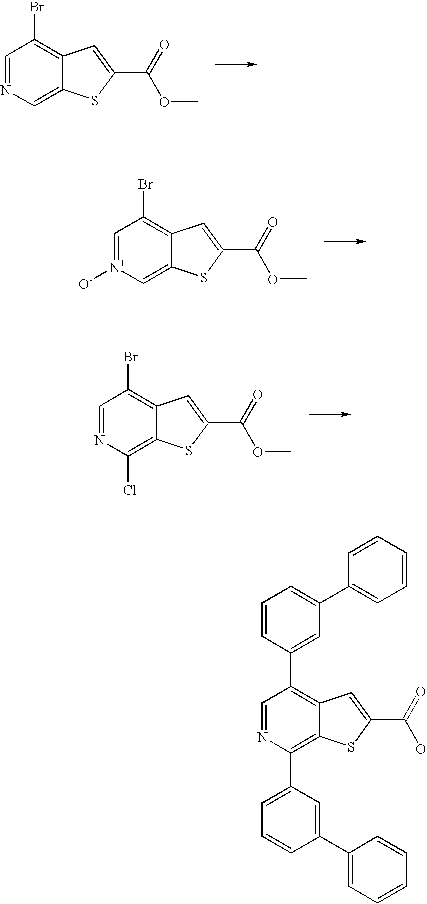

Following general procedure OO, to a solution of 4-bromo-thieno[2,3-c]pyridine-2-carboxylic acid methyl ester (0.500 g, 1.83 mmol) in DCM (20 mL) and methanol (5 mL) at about 0° C. was added mCPBA (0.411 g, 1.83 mmol). The resulting mixture was allowed to warm to room temperature and stirred for about 16 hours. The solvent was removed in vacuo and the resulting solid was dissolved in ethyl acetate (50 mL), washed with sodium bicarbonate (2×30 mL), brine (30 mL), dried over magnesium sulfate, and concentrated in vacuo to yield 4-bromo-6-oxy-thieno[2,3-c]pyridine-2-carboxylic acid methyl ester as a light yellow solid (0.527g, 1.83 mmol, no further purification was necessary); RP-HPLC (Table 1, Method i) m/z: (M+H)⁺ 288.0.

Following general procedure AAA, a solution of 4-bromo-6-oxy-thieno[2,3-c]pyridine-2-carboxylic acid methyl ester (0.527 g, 1.83 mmol) in phosphorus oxychloride (20 mL) was heated at reflux for about 5 hours. The reaction mixture was cooled to ambient temperature and poured carefully into ice water (150 mL) to quench excess reagent. The resulting precipitate was collected by filtration and dried in vacuo to yield 4-bromo-7-chloro-thieno[2,3-c]pyridine-2-carboxylic acid methyl ester (0.100 g, 0.326 mmol, no further purification was necessary); RP-HPLC (Hypersil C18, 5 μm, 100 Å, 15 cm; 5%-95% acetonitrile −0.05 M ammonium acetate over 15 min, 1 ml/min) Rₜ 13.06 min.

Following general procedure J, to a mixture of 4-bromo-7-chloro-thieno[2,3-c]pyridine-2-carboxylic acid methyl ester (0.100 g, 0.326 mmol), 3-biphenylboronic acid (0.064 g, 0.33 mmol), and sodium carbonate (0.086 g, 0.82 mmol) in dioxane (20 mL) and water (5 mL) was added tetrakis(triphenylphospine) palladium(0) (0.034 g, 0.030 mmol). The resulting mixture was heated at about 80° C. for about 6 hours. The solvent was removed in vacuo and the residue purified by preparative RP-HPLC (Hypersil C18, 5 μm, 100 Å, 15 cm; 15%-85% acetonitrile −0.05 M ammonium acetate over 30 min, 21 mL/min) to yield 4,7-bis-biphenyl-3-yl-thieno[2,3-c]pyridine-2-carboxylic acid (0.035 g, 0.072 mmol); RP-HPLC (Table 1, Method i) Rₜ 2.73 min; m/z: (M+H)⁻481.9.

### Preparation #96: C-[4-(Biphenyl-4-yloxy)-thieno[2,3-c]pyridine-2-yl]-methylamine Example 600

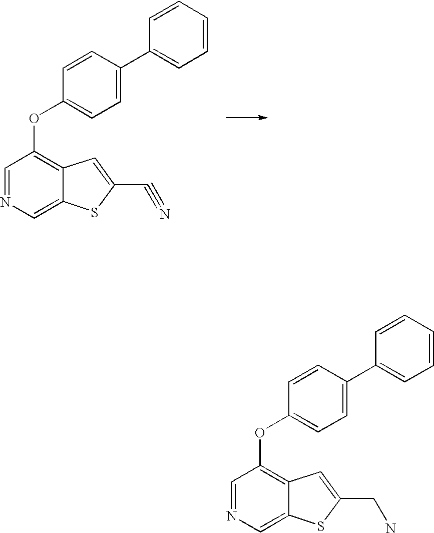

Following general procedure DD, to a solution of LS-selectride (0.150 mL, 0.152 mmol) in THF (2 mL) at about −78° C. was added dropwise a solution of 4-(biphenyl-4-yloxy)-thieno[2,3-c]pyridine-2-carbonitrile (0.050 g, 0.15 mmol) in THF (2 mL). The resulting solution was allowed to warm to room temperature and was stirred for about 6 hours. The solvent was removed in vacuo and the resulting oil was purified by preparative RP-HPLC (Hypersil C18, 5 μm, 100 Å, 15 cm; 15%-85% acetonitrile −0.05 M ammonium acetate over 30 min, 21 mL/min) to yield C-[4-(biphenyl-4-yloxy)-thieno[2,3-c]pyridine-2-yl]-methylamine (0.021 g, 0.063 mmol); RP-HPLC (Table 1, Method i) Rₜ 2.82 min; m/z: (M+H)⁺ 333.3.

### Preparation #97: 4-Biphenyl-3-yl-2-carboxy-1,6-dimethyl-1H-pyrrolo[2,3-c]pyridine-6-ium chloride, Example 601

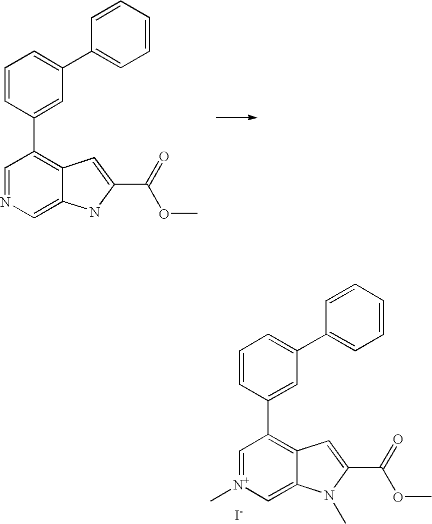

Following general procedure HH, to a solution of 4-biphenyl-3-yl-1H-pyrrolo[2,3-c]pyridine-2-carboxylic acid methyl ester (0.160 g, 0.487 mmol) in DMF (7 mL) was added sodium hydride (60% dispersion in mineral oil, 0.019 g, 0.49 mmol) and methyl iodide (0.03 mL, 0.5 mmol). The reaction was stirred at room temperature for about 3 hours. The solvent was removed in vacuo and the residue was dissolved in ethyl acetate (20 mL) and washed with water (20 mL) and brine (20 mL), dried over anhydrous magnesium sulfate, filtered, and concentrated in vacuo. The residue was purified by preparative RP-HPLC (Hypersil C 18, 5 μm, 100 Å, 15 cm; 10%-45% acetonitrile −0.05 M ammonium acetate over 30 min, 21 mL/min) to yield 4-biphenyl-3-yl-2-methoxycarbonyl-1,6-dimethyl-1H-pyrrolo[2,3-c]pyrdin-6-ium iodide as a yellow solid (0.080 g, 0.224 mmol) RP-HPLC (Table 1, Method i) Rₜ 2.86 min; m/z: (M+H)⁻357.2.

To 4-biphenyl-3-yl-2-methoxycarbonyl-1,6-dimethyl-1 H-pyrrolo[2,3-c]pyrdin-6-ium iodide (0.080 g, 0.22 mmol) was added 2N aqueous sodium hydroxide solution (20 mL). The reaction mixture was heated at about 100° C. for about 6 hours. The reaction mixture was cooled to ambient temperature and acidified with 2N HCl. The resulting precipitate was collected by filtration to yield 4-biphenyl-3-yl-2-carboxy-1,6-dimethyl-1H-pyrrolo[2,3-c]pyridine-6-ium chloride (0.033g, 0.096 mmol); RP-HPLC (Table 1, Method i) Rₜ 1.49 min; m/z: (M+H)⁻ 343.2.

### Preparation #98: 4-[3-(Carbamoylmethyl-amino)-phenyl]-thieno[2,3-c]pyridine-2-carboxylic acid amide, Example 602

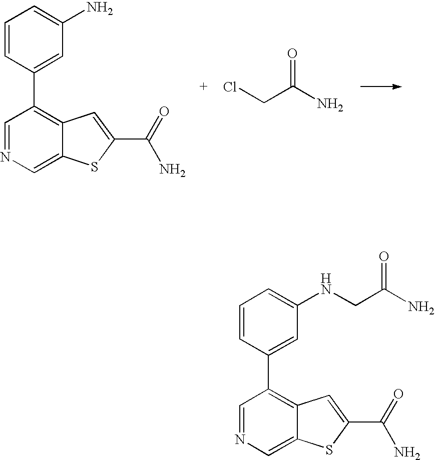

Following general procedure HH, to a solution of 4-(3-amino-phenyl)-thieno[2,3-c]pyridine-2-carboxylic acid amide (0.101 g, 0.371 mmol) in DMF (8 mL) was added 2-chloroacetamide (0.035 g, 0.37 mmol) and cesium carbonate (0.302 g, 0.928 mmol). The mixture was allowed to stir at about 80° C. for about 7 days. The solvent was removed under reduced pressure until about 2 mL of the reaction mixture remained. The mixture was purified on a normal phase silica column using 10 % methanol: EtOAc as the mobile phase to afford 4-[3-(carbamoylmethyl-amino)-phenyl]-thieno[2,3-c]pyridine-2-carboxylic acid amide (0.020 g, 0.061 mmol) as a yellow-orange solid; RP-HPLC (Table 1, Method a) Rₜ 6.69 min; n/z: (M+H)⁺ 327.

### Preparation #99: [3-(2-Carbamoyl-thieno[2,3-c]pyridin-4-yl)-phenyl]-carbamic acid isopropyl ester, Example 603

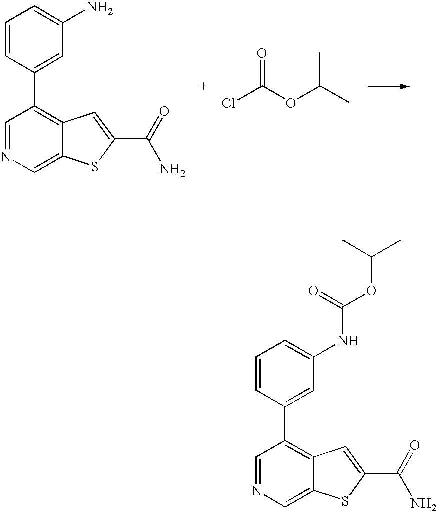

Following general procedure HH, a mixture of 4-(3-amino-phenyl)-thieno[2,3-c]pyridine-2-carboxylic acid amide (0.099 g, 0.37 mmol) and isopropyl chloroformate (1M in toluene) (0.37 mL, 0.37 mmol) was stirred in pyridine (8 mL) at ambient temperature for about 12 hours. The solvent was removed under reduced pressure and the residue was then triturated in ether to provide [3-(2-carbamoyl-thieno[2,3-c]pyridin-4-yl)-phenyl]-carbamic acid isopropyl ester (0.023 g, 0.065 mmol) as a white solid; RP-HPLC (Table 1, Method a) Rₜ 8.98 min; m/z: (M+H)⁺ 356.

### Preparation #100: 4-(Biphenyl-4-ylamino)-pyrrolo[2,3-c]pyridine-1,2-dicarboxylic acid 1-tert-butyl ester 2-methyl ester

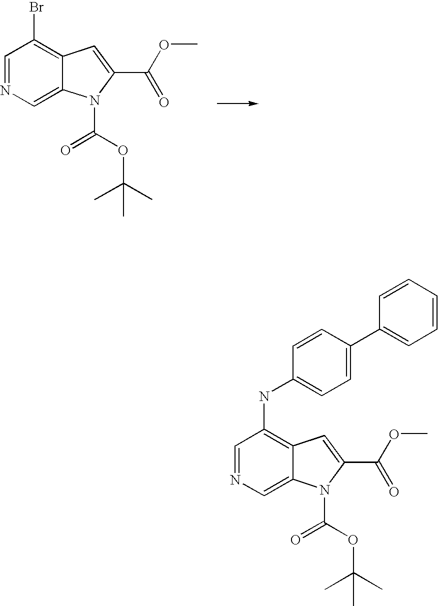

Following general procedure I, to a mixture of 4-bromo-pyrrolo[2,3-c]pyridine-1,2-dicarboxylic acid 1-tert-butyl ester 2-methyl ester (3.2 g, 9.0 mmol), cesium carbonate (5.85 g, 18 mmol), 4-phenyl aniline (1.67 g, 9.9 mmol), Pd₂(dba)₃ (0.825 g, 0.90 mmol) and Xantphos (0.521 g, 0.90 mmol) was added anhydrous 1,4-dioxane (45 mL) which had been degassed with nitrogen for 30 min prior to use. The mixture was heated at about 100° C. for about 20 hours. The reaction mixture was cooled to ambient temperature, the solvent was removed in vacuo, and the resulting solid was diluted with EtOAc (100 mL) and passed through a plug of celite. The filtrate was washed with water (3×50 mL) and dried over sodium sulfate. The solvents were removed in vacuo and the resulting oil was purified by silica gel chromatography using a mixture of heptane/AcOEt (7:3) as eluent to provide 4-(biphenyl-4-ylamino)-pyrrolo[2,3-c]pyridine-1,2-dicarboxylic acid 1-tert-butyl ester 2-methyl ester as a bright yellow solid (1.77 g, 44 %). ¹H NMR (d₆-DMSO, 400 MHz): δ 8.77 (s, 1H), 8.66 (s, 1H), 8.36 (s, 1H), 7.65 (m, 4H), 7.44 (m, 3H), 7.28 (m, 3H), 3.89 (s, 3H), and 1.59 (s, 9H); RP-HPLC (Table 1, Method n): Rₜ=5.49 min; m/z: (M+H)⁺ 443.9.

### Preparation #101: 4-(Biphenyl-4-ylamino)-thieno[2,3-c]-pyridine-2-carboxylic acid biphenyl-1-4-ylamide, Example 604

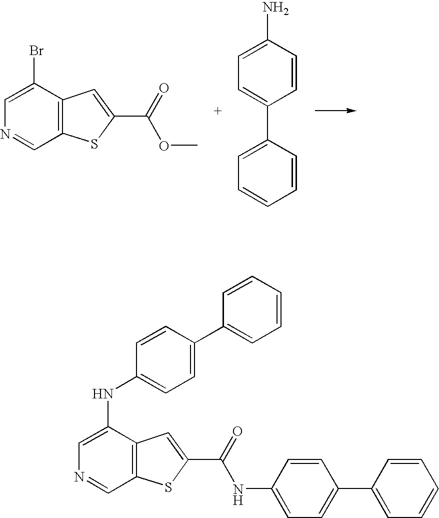

Following general procedure I, To a solution of 4-bromo-thieno[2,3-c]pyridine-2-carboxylic acid methyl ester (prepared using general procedures A and B) (0.100 g, 0.368 mmol) in anhydrous toluene (10 mL) was added biphenyl-4-ylamine (0.186 g, 1.10 mmol), sodium tert-butoxide (0.706 g, 7.36 mmol), and 1,1′-bis(diphenylphosphino)ferrocene (0.041 g, 0.074 mmol). The mixture was stirred and nitrogen gas was bubbled through the suspension for about five minutes at ambient temperature.

Tris(dibenzylideneacetone)dipalladium (0) (0.016 g, 0.018 mmol) was added. Nitrogen gas was then bubbled through the resulting mixture for five minutes and the reaction was heated at about 110° C. for about 18 hours. The reaction mixture was cooled to ambient temperature, and the solvent was removed in vacuo. The crude oil was taken up in DMSO and purified via preparative RP-HPLC (20% to 100% acetonitrile/0.05M aqueous ammonium acetate, buffered to pH 4.5, over 25 min, then 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to afford 4-(biphenyl-4-ylamino)-thieno[2,3-c]-pyridine-2-carboxylic acid biphenyl-1-4-ylamide (0.065 g, 0.13 mmol) as a tan solid; RP-HPLC (Table 1, Method m) Rₜ5.28 min; m/z: (M+H)⁺ 498.3, m/z: (M+H)⁻ 496.2.

### Preparation #102: 4-(Biphenyl-4-ylamino)-thieno[2,3-c]pyridine-2-carboxylic acid amide, Example 62

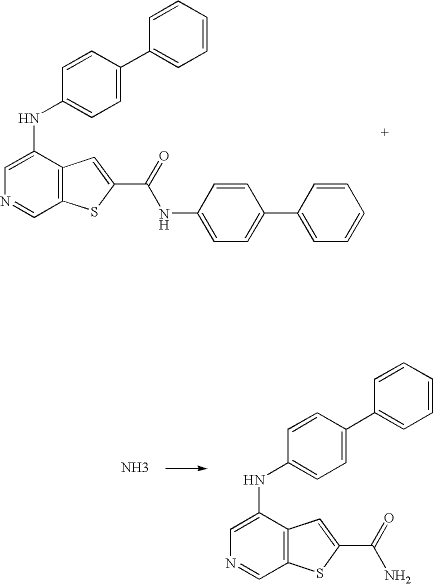

Following general procedure BB, a suspension of 4-(biphenyl-4-ylamino)-thieno[2,3-c]-pyridine-2-carboxylic acid biphenyl-1-4-ylamide (0.06 g, 0.1 mmol) in 7N ammonia in methanol (2 mL) was heated at about 70° C. for about 18 hours. The reaction mixture was cooled to ambient temperature, and the solvent was removed in vacuo. The crude oil was taken up in DMSO and purified via preparative RP-HPLC (20% to 100% acetonitrile/0.05M aqueous ammonium acetate, buffered to pH 4.5, over 25 min, then 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to afford 4-(biphenyl-4-ylamino)-thieno[2,3-c]pyridine-2-carboxylic acid amide (0.002 g, 0.006 mmol) as a tan solid; RP-HPLC (Table 1, Method m) Rₜ 3.42 min; m/z: (M+H)⁺ 346.3, m/z: (M+H)⁻ 344.2.

### Preparation #103: 4-(Biphenyl-4-ylamino)-thieno[2,3-c]-pyridine-2-carboxylic acid biphenyl-1-4-ylamide, Example 605

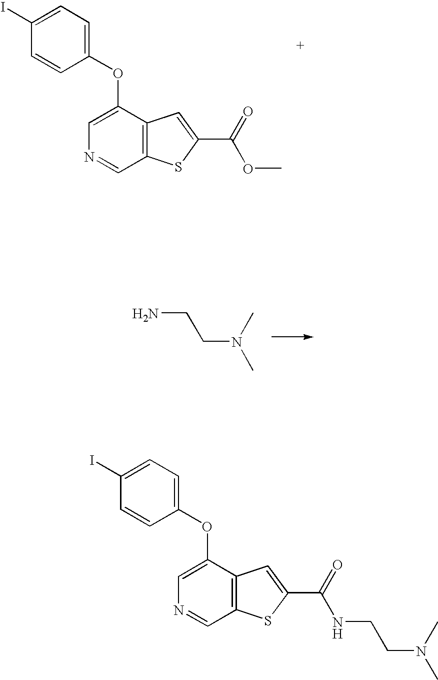

Following general procedure BB, to a solution of 4-(4-iodo-phenoxy)-thieno[2,3-c]pyridine-2-carboxylic acid methyl ester (prepared using general procedures A and D) (0.015 g, 0.036 mmol) in anhydrous dioxane (1 mL) was added N*1*,N*1*-dimethyl-ethane-1,2-diamine (0.50 mL, 6.8 mmol). The reaction mixture was heated to about 105° C. for about 4 hours then cooled to ambient temperature and the solvent was removed in vacuo. The crude oil was taken up in DMSO and purified via preparative RP-HPLC (20% to 100% acetonitrile (0.1% trifluoroacetic acid)/0.05M aqueous ammonium acetate, buffered to pH 4.5, over 25 min, then 100% acetonitrile (0.1% trifluoroacetic acid/0.05M aqueous ammonium acetate, buffered to pH 4.5, over 5 min, at 81 mL/min; λ=254 nm; Hyperprep® HS C18, 8 μm, 250×21.2 mm column) to afford 4-(4-iodo-phenoxy)-thieno[2,3-c]pyridine-2-carboxylic acid (2-dimethylamino-ethyl)-amide (0.017 g, 0.036 mmol) as a white solid; RP-HPLC (Table 1, Method m) Rₜ 2.85 min; m/z: (M+H)⁺ 468.0, m/z: (M−H)⁻ 465.9.

4-(4-Iodo-phenylamino)-thieno[2,3-c]pyridine-2-carboxylic acid (3-morpholin-4-yl-propyl)-amide, Example 596 was similarly prepared as a white solid; RP-HPLC (Table 1, Method m) Rₜ 2.96 min; m/z: (M+H)⁺ 524.0, m/z: (M−H)⁻ 522.0.

### Preparation #104: 4-[4-(2-Chloro-acetylamino)-phenyl]-thieno[2,3-c]pyridine-2-carboxylic acid amide

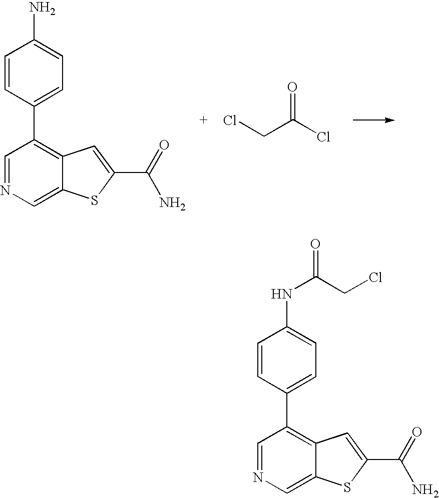

Following general procedure HH, to a cooled (0-5° C.) solution of 4-(4-amino-phenyl)-thieno[2,3-c]pyridine-2-carboxylic acid amide (1.01 g, 3.71 mmol) and sodium carbonate (1.45 g, 13.7 mmol) in dichloromethane (40 mL) was added chloroacetyl chloride (740 μL, 9.3 mmol) dropwise. The mixture was allowed warm up slowly to room temperature, and was stirred for 4 days. The precipitate was collected via vacuum filtration and triturated with water. The resulting solid was dried to yield 1.10 g (3.18 mmol) of the desired product, 4-[4-(2-Chloro-acetylamino)-phenyl]-thieno[2,3-c]pyridine-2-carboxylic acid amide, as slightly green solid. RP-HPLC (table 1, method a) Rₜ 8.00 min; m/z: (M+H)⁺ 346.

### Preparation #105: 4-(2-Carbamoyl-thieno[2,3-c]pyridine-4-yl)-benzoic acid

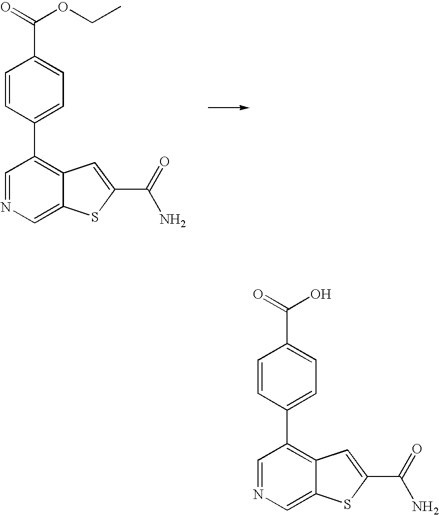

Following general procedure E, a solution of 4-(2-Carbamoyl-thieno[2,3-c]pyridin-4-yl)-benzoic acid ethyl ester (0.462 g, 1.42 mmol) and lithium hydroxide monohydrate (0.065 g, 1.56 mmol) in 1:1 mixture of dioxane and water was allowed to stir for 5 days at room temperature. The grey suspension was acidified to pH of 2 by adding 2M hydrochloric acid, at which point white precipitates formed. The precipitate was collected via vacuum filtration and dried to give 0.328 g (1.10 mmol) of the desired product, 4-(2-Carbamoyl-thieno[2,3-c]pyridine-4-yl)-benzoic acid, as white brittle solid. RP-HPLC (table 1, method a) Rₜ 6.38 min; m/z: (M+H)⁺ 399.

### General procedure CCC: Debromination of an aryl bromide

To a mixture of an aryl bromide in an organic solvent (diethyl ether, 1,2-dimethoxyethane, dioxane; preferably 1,2-dimethoxyethane) and aqueous inorganic base (potassium carbonate, sodium hydrogen carbonate, cesium carbonate; preferably cesium carbonate) (1-5 equivalents, preferably 1 equivalent) was added a palladium catalyst (tetrakis-triphenylphospine palladium, dihydrogen 2-dichlorobis(di-tert-butylphosphinito-kP)dipalladate; preferably dihydrogen 2-dichlorobis(di-tert-butylphosphinito-kP)dipalladate) (0.01 to 0.5 equivalents, preferably 0.2 equivalents). The mixture was stirred under a nitrogen atmosphere at about 20-100° C. (preferably about 85° C.) for about 1 to 72 h (preferably about 48 h). The solvent was removed in vacuo to afford the product which can be further purified by chromatography or crystallization.

Illustration of General Procedure CCC

### Preparation #106: 4-(Biphenyl-4-ylamino)-thieno[2,3-c]-pyridine-2-carboxylic acid biphenyl-1-4-ylamide, Example 606

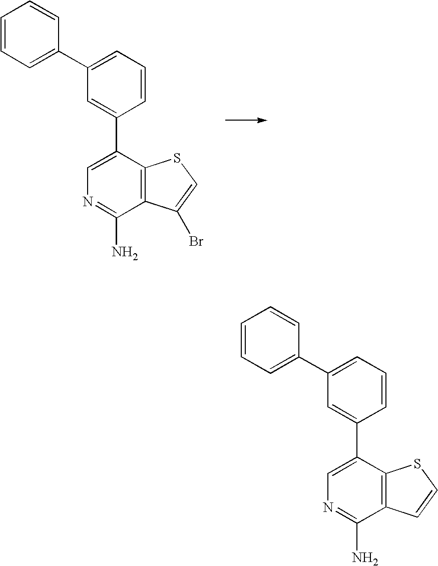

To a suspension of 7-biphenyl-3-yl-3-bromo-thieno[3,2-c]pyridin-4-ylamine (prepared via method J) (0.035 g, 0.092 mmol) in anhydrous 1,2-dimethoxyethane (3.0 mL) and water (1.0 mL) was added cesium carbonate (0.03 g, 0.09 mmol), and dihydrogen dichlorobis(di-tert-butylphosphinito-KP)dipalladate(2-) (0.01 g, 0.02 mmol). The mixture was stirred and nitrogen gas was bubbled through the suspension for about five minutes at ambient temperature. The reaction was heated at about 85° C. for about 48 hours. The reaction mixture was cooled to ambient temperature, and the solvent was removed in vacuo. The crude oil was taken up in DMSO and purified via preparative RP-HPLC (20% to 100% acetonitrile/0.05M aqueous ammonium acetate, buffered to pH 4.5, over 25 min, then 100% acetonitrile/0.05M aqueous ammonium acetate, buffered to pH 4.5, over 5 min, at 81 mL/min; λ=254 nm; Hyperprep® HS C18, 8 μm, 250×21.2 mm column) to afford 7-biphenyl-3-yl-thieno[3,2-c]pyridin-4-ylamine (0.0035 g, 0.012 mmol) as a tan solid; RP-HPLC (Table 1, Method m) Rₜ 3.90 min; m/z: (M+H)⁺ 303.2.

### Preparation #107: Biphenyl-4-yl-[2-(2H-pyrazol-3-yl)-thieno[2,3-c]pyridin-4-yl]-amine, Example 607

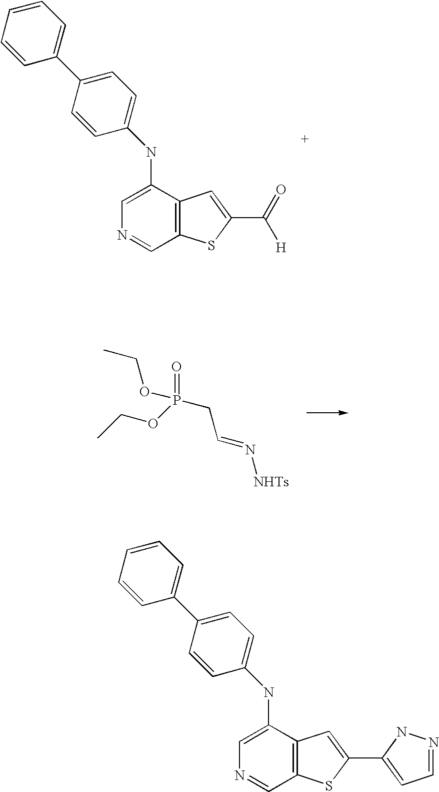

Using the general protocol described by Almirante, N et al; Tetrahedron Letters, (1998), 39, 3287-3290, a suspension of NaH (60% in mineral oil, 0.046 g, 1.134 mmol) in anhydrous THF (10 mL) at 0° C., was added to a solution of diethoxyphosphorylacetaldehyde tosylhydrazone (0.198 g, 0.568 mmol) in anhydrous THF (5 mL). After stirring the mixture for about 30 minutes at 0° C., a solution of 4-(biphenyl-4-ylamino)-thieno[2,3-c]pyridine-2-carbaldehyde (prepared in preparation #91, 0.125 g, 0.378 mmol) in anhydrous THF (5 mL) was added to the reaction mixture. The reaction was then left to warm to ambient temperature for about two hours, then heated to reflux for a period of one hour. The reaction was cooled to ambient temperature and poured into a solution of 5% aqueous NaH₂PO₄ (50 mL). The resulting mixture was extracted with ethyl acetate (3×25 mL). The organic layers were combined and the solvent removed under reduced pressure. The crude material was taken up in DMSO and purified via preparative RP-HPLC (20% to 100% acetonitrile/0.05M aqueous ammonium acetate, buffered to pH 4.5, over 30 min, at 21 mL/min; λ=254 nm; Hypersil C18, 100 Å, 8 □m, 250×21.1 mm column) to afford biphenyl-4-yl-[2-(2H-pyrazol-3-yl)-thieno[2,3-c]pyridin-4-yl]-amine (0.043 g, 0.116 mmol) as a yellow solid; RP-HPLC (Table 1, Method b) Rₜ 8.71 min; m/z: (M+H)⁺ 369.2.

### Preparation #108: Thieno[2,3-c]pyridine-2-carboxylic acid methyl ester, Example 608

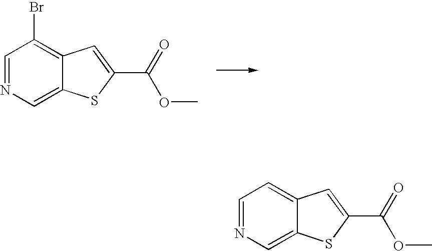

Following general procedure PP, 4-Bromo-thieno[2,3-c]pyridine-2-carboxylic acid methyl ester (2.00 g, 7.35 mmol) was dissolved in ethanol (50 ml) containing a suspension of 10% Pd on Carbon (200 mg) and the mixture was shaken two days under about 40 psi of H₂. The reaction was filtered through celite and concentrated and then the crude was dissolved in EtOAc, washed with sat. NaHCO₃ solution, dried (MgSO4) and concentrated. The residue was further purified on silica gel using CH₂Cl₂:EtOAc/9:1 as eluent. Product fractions were combined and concentrated to yield thieno[2,3-c]pyridine-2-carboxylic acid methyl ester (720 mg, 3.73 mmol) as a white solid); RP-HPLC (Table 1, Method i) Rₜ=0.62 min; m/z: (M+H)⁺ 194.

### Preparation #109: 7-Biphenyl-4-ylmethyl-thieno[2,3-c]pyridine-2-carboxylic acid methyl ester, Example 609

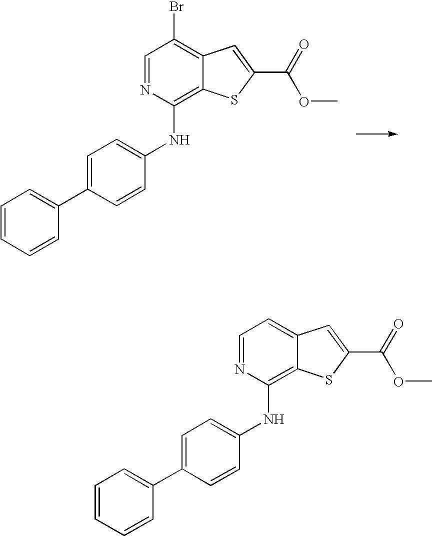

Following general procedure PP, 7-(Biphenyl-4-ylamino)-4-bromo-thieno[2,3-c]pyridine-2-carboxylic acid methyl ester (179 mg, 0.408 mmol) ) was dissolved in ethanol (20 ml) containing a suspension of 10% Pd on Carbon (200 mg) and the mixture was shaken overnight under about 40 psi of H₂. The reaction was filtered through celite and concentrated. The crude was further purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column). Product fractions were combined and concentrated to yield 7-Biphenyl-4-ylmethyl-thieno[2,3-c]pyridine-2-carboxylic acid methyl ester (46 mg, 0.13 mmol) as an off-white solid); RP-HPLC (Table 1, Method j) Rₜ=5.32 min; m/z: (M+H)⁺ 361.

### Preparation #110: 4-(Biphenyl-4-ylamino)-7-phenyl-thieno[2,3-c]pyridine-2-carboxylic acid amide, Example 610

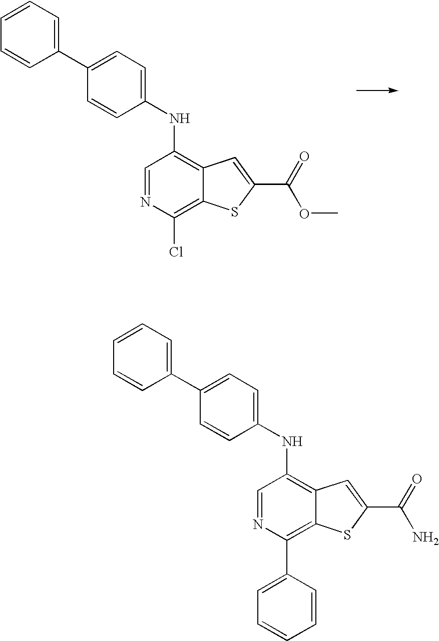

Following general procedure J, to a mixture of 4-(Biphenyl-4-ylamino)-7-chloro-thieno[2,3-c]pyridine-2-carboxylic acid methyl ester (prepared using general procedures A, B, N-oxidation chlorination, and I) (0.050 g, 0.127 mmol), 1-biphenylboronic acid (0.031 g, 0.25 mmol) and cesium carbonate (123 mg, 0.39 mmol) was added [1,1′-Bis(diphenylphosphino)ferrocene]dichloropalladium(11), complex with dichloromethane (4.90 mg, 0.006 mmol) at room temperature under an atmosphere of nitrogen. The reaction mixture was heated by microwave at about 150° C. for about 10 min. The mixture was allowed to cool to ambient temperature, filtered through a silica gel pad, and concentrated. The residue dissolved in 7M NH₃/methanol and heated in a sealed tube at 70° C. overnight. Solvents were evaporated and the residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-(Biphenyl-4-ylamino)-7-phenyl-thieno[2,3-c]pyridine-2-carboxylic acid amide (6.0 mg, 0.014 mmol) as a yellow solid; RP-HPLC (Table 1, Method i) Rₜ=3.38 min; m/z: (M+H)⁺ 422.

### Preparation #111: 4-(Biphenyl-4-ylamino)-7-methyl-thieno[2,3-c]pyridine-2-carboxylic acid amide, Example 611

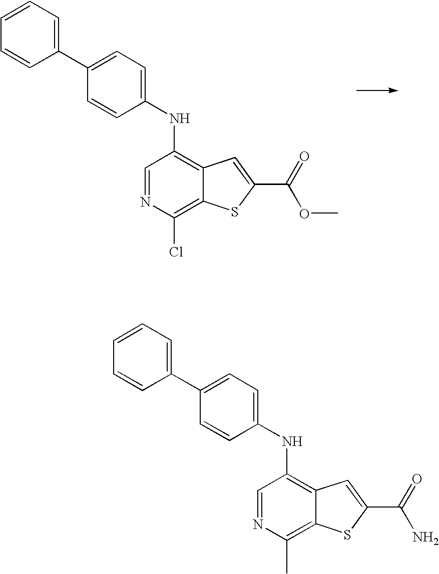

Following general procedure J, to a mixture of 4-(Biphenyl-4-ylamino)-7-chloro-thieno[2,3-c]pyridine-2-carboxylic acid methyl ester (prepared using general procedures A, B, N-oxidation chlorination, and I) (0.050 g, 0.127 mmol), Trimethylboroxine (97.5 mg, 0.39 mmol) and cesium carbonate (41.0 mg, 0.13 mmol) was added [1,1′-Bis(diphenylphosphino)ferrocene]dichloropalladium(11), complex with dichloromethane (4.90 mg, 0.006 mmol) at room temperature under an atmosphere of nitrogen. The reaction mixture was at about 90° C. for about 18 hr. The mixture was allowed to cool to ambient temperature, filtered through a celite pad, and concentrated. The residue dissolved in 7M NH₃/methanol and heated in a sealed tube at 70° C. overnight. Solvents were evaporated and the residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-(Biphenyl-4-ylamino)-7-methyl-thieno[2,3-c]pyridine-2-carboxylic acid amide (17 mg, 0.045 mmol) as a yellow solid; RP-HPLC (Table 1, Method i) Rₜ=2.61 min; m/z: (M+H)⁺ 360.

### Preparation #112: 4-(Biphenyl-4-ylamino)-7-cyano-thieno[2,3-c]pyridine-2-carboxylic acid methyl ester

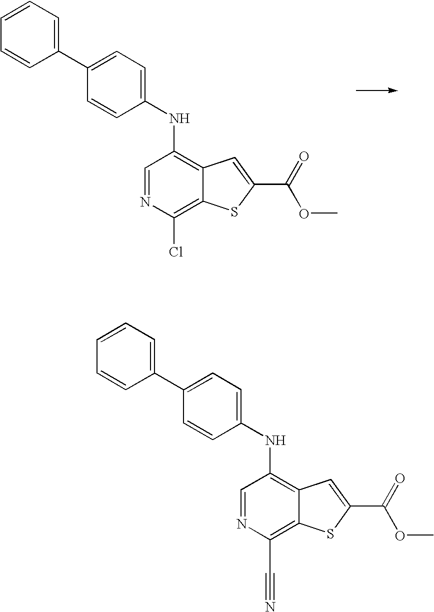

Following the literature procedure of A. Hallberg and M. Alterman, J. Org. Chem. (2000), 65, 7984-7989, a mixture of 4-(Biphenyl-4-ylamino)-7-chloro-thieno[2,3-c]pyridine-2-carboxylic acid methyl ester (prepared using general procedures A, B, N-oxidation chlorination, and I) (0.050 g, 0.127 mmol), Zn(CN)₂ (14.9 mg, 0.127 mmol) and tetrakis(triphenylphosphine) palladium(0) were combined in DMF (1.0 ml) and heated by microwave at 175° C. for 20 min. The mixture was cooled to ambient temperature, filtered through a celite pad, and concentrated to yield 4-(Biphenyl-4-ylamino)-7-cyano-thieno[2,3-c]pyridine-2-carboxylic acid methyl ester as a yellow solid which could be used without further purification; RP-HPLC (Table 1, Method i) Rₜ=4.18 min; m/z: (M+H)⁺ 384.

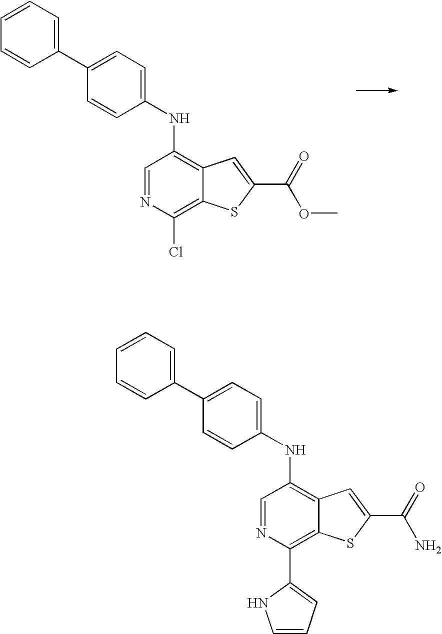

### Preparation #113: 4-(Biphenyl-4-ylamino)-7-(1H-pyrrol-2-yl)-thieno[2,3-c]pyridine-2-carboxylic acid amide, Example 612

Following general procedure J, to a mixture of 4-(Biphenyl-4-ylamino)-7-chloro-thieno[2,3-c]pyridine-2-carboxylic acid methyl ester (prepared using general procedures A, B, N-oxidation chlorination, and I) (0.050 g, 0.127 mmol), 1-(t-Butoxycarbonyl)pyrrole-2-boronic acid (79.4 mg, 0.375 mmol), cesium carbonate (123.0 mg, 0.39 mmol) and H₂O (200 ul) in p-dioxane (2 ml) was added [1,1′-Bis(diphenylphosphino)ferrocene]dichloropalladium(11), complex with dichloromethane (4.90 mg, 0.006 mmol) under nitrogen. The reaction mixture was heated by microwave at about 150° C. for about 20 min. The mixture was cooled to ambient temperature, filtered through a celite pad, and concentrated. The residue dissolved in 7M NH₃/methanol and heated in a sealed tube at 70° C. overnight. Solvents were evaporated and the residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-(Biphenyl-4-ylamino)-7-(1H-pyrrol-2-yl)-thieno[2,3-c]pyridine-2-carboxylic acid amide (4.0 mg, 0.01 mmol) as a yellow solid; RP-HPLC (Table 1, Method i) Rₜ=3.12 min; m/z: (M−H)⁻ 409.

### Preparation #114: 4-(Biphenyl-4-ylamino)-7-carbamoyl-thieno[2,3-c]pyridine-2-carboxylic acid, Example 613

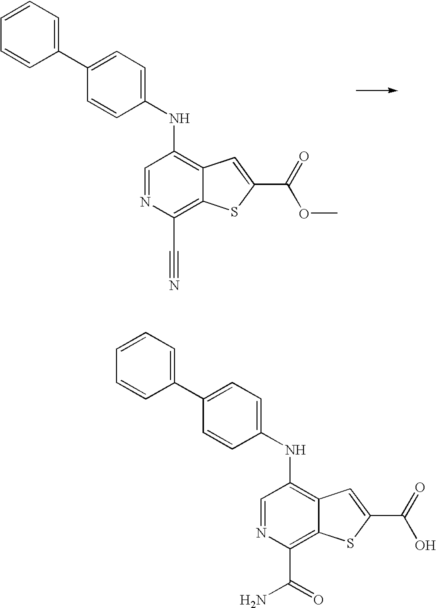

Following general procedure H, 4-(Biphenyl-4-ylamino)-7-cyano-thieno[2,3-c]pyridine-2-carboxylic acid methyl ester (100 mg, 0.254 mmol) was combined with polyphoshoric acid (about 2.0 ml) and the mixture was heated at about 150° C. for about 1 hour. The mixture was cooled, diluted with H₂O (about 20 ml) and neutralized with 2M NaOH. The intermediate was filtered off, dried under vacuum and then heated for about 1 hour with 2N NaOH (1.0 ml) in p-dioxane (6 ml). Solvents were evaporated and the residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-(Biphenyl-4-ylamino)-7-carbamoyl-thieno[2,3-c]pyridine-2-carboxylic acid (41 mg, 0.11 mmol) as a yellow solid); RP-HPLC (Table 1, Method i) Rₜ=1.78 min; m/z: (M−H)⁻ 388.

### Preparation #115 2-Methoxycarbonyl-thieno[2,3-c]pyridin-4-yl-ammonium; chloride, Example 614

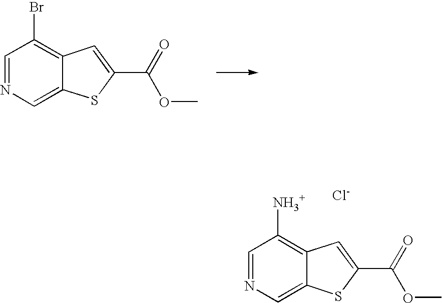

Following general procedure I, 4-Bromo-thieno[2,3-c]pyridine-2-carboxylic acid methyl ester (2.72 g, 10.0 mmol), Benzophenone imine (1.99g, 11.0 mmol), Cesium carbonate (3.76 g, 11.0 mmol), tris(dibenzylideneacetone)dipalladium(0) (228 mg, 0.25 mmol) and 4,5-Bis(diphenylphosphino)-9,9-dimethylxanthene (289 mg, 0.50 mmol) were combined in p-dioxane (40 ml) under nitrogen in a sealed vessel and heated at about 100° C. for about 3 days. The reaction was cooled, filtered and 2M HCl (25 ml) was added and the reaction was stirred for about 30 min. The product was filtered off and washed with p-dioxane (3×5 ml) and then dried to yield 2-Methoxycarbonyl-thieno[2,3-c]pyridin-4-yl-ammonium; chloride (2.17 g, 8.89mmol) as a yellow solid; RP-HPLC (Table 1, Method i) Rₜ=1.47 min; m/z: (M+H)⁺ 209.

### Preparation #116: 7-Amino-4-biphenyl-3-yl-thieno[2,3-c]pyridine-2-carboxylic acid methyl ester

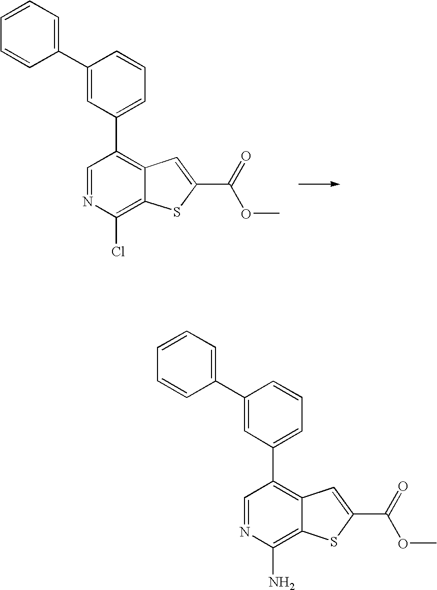

Following general procedure I, 4-Biphenyl-3-yl-7-chloro-thieno[2,3-c]pyridine-2-carboxylic acid methyl ester (125 mg, 0.33 mmol) ), Benzophenone imine (60.3 uL, 0.36 mmol), Cesium carbonate (130 mg, 0.40 mmol), tris(dibenzylideneacetone)dipalladium(0) (7.30 mg, 0.012 mmol) and 4,5-Bis(diphenylphosphino)-9,9-dimethylxanthene (14.0 mg, 0.024 mmol) were combined in p-dioxane (3 ml) under nitrogen in a sealed vessel and heated at about 100° C. for about 5 hours. The reaction was cooled, filtered through celite and 2M HCl (1.0 ml) was added and the reaction was stirred at room temperature for about 1 hour. The reaction was concentrated under reduced pressure, triturated several times with ether (5 ml) and the residue was used, as is, in the following step.

### General procedure DDD: Cyanation of a pyridine N-oxide

A pyridine N-oxide, a cyanating agent (sodium cyanide, copper (1) cyanide, or trimethylsilyl cyanide, preferably trimethylsilylcynide) (1-5 equivalents, preferably 2.5 equivalents) and an organic base (Hunig's base, triethylamine, or morpholine, preferably triethylamine) (1-30 equivalents, preferably 15 equivalents) were combined in an organic solvent (DMF, CH₃CN or dioxane, preferably CH₃CN) under N₂ and heated at about 40-110° C. (preferably about 85° C.) for about 0.5-5 h (preferably about 2 h). The solvent was removed under reduced pressure and the residue was treated with a strong acid (trifluoroacetic acid, sulfuric acid, preferably trifluoroacetic acid) (1-1,000 equivalents, preferably 100 equivalents) and a silyl hydride (preferably iPr₃SiH) (1-30 equivalents, preferably 2.5 equivalents) at about 0-50° C. (preferably about 25° C.) for about 0.1 to 10 h (preferably about 0.2 h). The solvent was removed in vacuo to afford the product which can be further purified by chromatography or crystallization.

Illustration of General Procedure DDD

### Preparation #117: 4-(Biphenyl-4-ylamino)-7-cyano-thieno[2,3-c]pyridine-2-carboxylic acid, Example 615

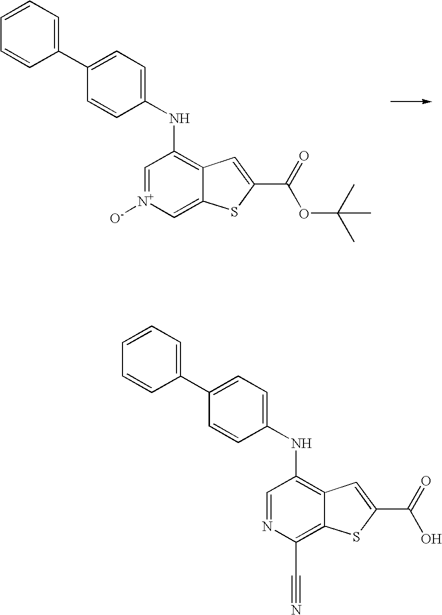

4-(Biphenyl-4-ylamino)-6-oxy-thieno[2,3-c]pyridine-2-carboxylic acid tert-butyl ester (105 mg, 0.25 mmol), Trimethylsilyl cyanide (80.0 ul, 0.60 mmol) and Triethylamine (51.6 ul, 0.37 mmol) were combined in CH₃CN (5.0 ml) under N₂ in a sealed tube and heated at about 82° C. for about 2 hours. Solvents were removed under reduced pressure the residue was treated with Trifluoroacetic acid (2 ml) containing iPr₃SiH (123 uL, 0.60 mmol) for about 15 min at ambient temperature. The mixture was concentrated under reduced pressure and the residue was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 4-(Biphenyl-4-ylamino)-7-cyano-thieno[2,3-c]pyridine-2-carboxylic acid as (20 mg, 0.054 mmol) a yellow solid; RP-HPLC (Table 1, Method i) Rₜ=1.95 min; m/z: (M−H)⁻ 370.

### General procedure EEE: Mitsunobu coupling

A solution of an alcohol, and a phosphine (preferably triphenylphosphine) (1-5 equivalents, preferably 1 equivalent) in an organic solvent (THF, dioxane, diethyl ether, preferably THF) was cooled to about −30 to 20° C. (preferably about 0° C.) under N₂ and a azodicarboxylate (diethyl azodicarboxylate, diisopropyl azodicarboxylate, preferably diisopropyl azodicarboxylate) (1-5 equivalents, preferably 1 equivalent) was added. The reaction was stirred for about 0.1 to 2 h (preferably about 0.2 h) then a solution of a hydroxyl isoindole-1,3-dione (1-5 equivalents, preferably 1 equivalent) in an organic solvent (THF, dioxane, diethyl ether, preferably THF) was added dropwise over about 0.1 to 2 h (preferably about 0.2 h). The reaction was stirred at about 0-50° C. (preferably about 20° C.) for about 1-48 h (preferably about 16 h). The solvent was removed in vacuo to afford the product which can be further purified by chromatography or crystallization.

Illustration of General Procedure EEE

### Preparation #118: 2-(2-tert-Butoxy-ethoxy)-isoindole-1,3-dione

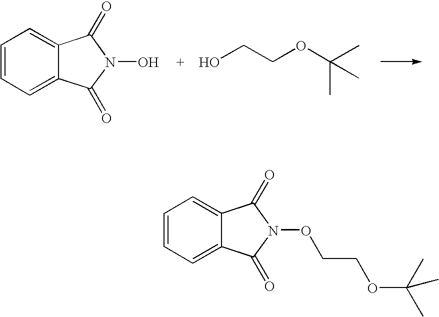

A solution of 2-tert-Butoxy-ethanol (1.18 g, 10.0 mmol) and Tiphenylphosphine (2.62 g, 10.0 mmol) in THF (25 ml) was cooled to 0° C. under N₂ and Diisopropyl azodicarboxylate (1.97 ml, 10.0 mmol) was added dropwise while maintaining reavtion temperature below 5° C. The reaction was stirred an additional 15 min at 0° C. and then a solution of 2-Hydroxy-isoindole-1,3-dione (1.63 g, 10.0 mmol) in THF (40 ml) was added dropwise over about 20 min. The reaction was allowed to come slowly to room temperature with stirring overnight. Solvents were removed under reduced pressure and the mixture was purified by silica gel chromatography using 7:3 Heptane:EtOAc as eluant. Product fractions were combined and concentrated to 1.48 g (5.63 mmol) of an oil which crystallized on standing. ¹H NMR (d₆-DMSO, 400 MHz): δ 7.86 (4H, s), 4.24-4.28 (2H, m), 3.60-3.64 (2H, m), 0.99 (9H, s); RP-HPLC (Table 1, Method m) Rₜ=3.45 min.

### General procedure FFF: Phthalimide deprotection

An N-substituted phthalimide was dissolved in an organic solvent (diethyl ether, CH₂Cl₂ or dioxane, preferably CH₂Cl₂) and a hydrazine (ethylhydrazine, methylhydrazine, preferably methylhydrazine) (1-10 equivalents, preferably 1 equivalent) was added. The reaction was stirred at about 0-50° C. (preferably about 20° C.) for about 1-48 h (preferably about 16 h) at room temperature. The solvent was removed in vacuo to afford the product which can be further purified by chromatography or crystallization.

Illustration of General Procedure FFF

### Preparation #119: O-(2-tert-Butoxy-ethyl)-hydroxylamine

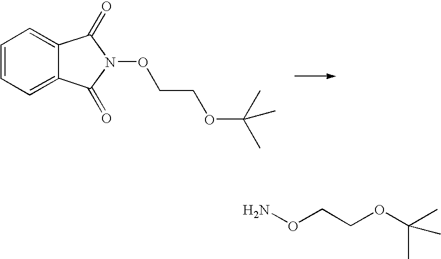

2-(2-tert-Butoxy-ethoxy)-isoindole-1,3-dione (263mg, 1.0 mmol) was dissolved in CH₂CL₂ (3 ml) and Methylhydrazine (52.6 uL, 1.0 mmol) was added at room temperature. The reaction was allowed to stir overnight at ambient temperature and was then used crude without purification.

### Preparation #120: 4-(5-Pyridin-2-yl-thiophen-2-ylamino)-thieno[2,3-c]pyridine-2-carboxylic acid methyl ester

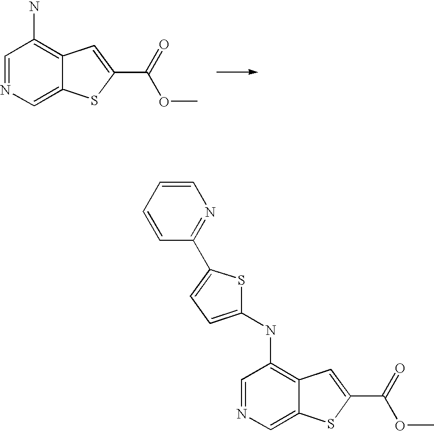

Following general procedure I, 2-Methoxycarbonyl-thieno[2,3-c]pyridin-4-yl-ammonium; chloride (50 mg, 0.20 mmol), 2-(5-Bromo-thiophen-2-yl)-pyridine (54 mg, 0.23 mmol), Cesium carbonate (220 mg, 0.7 mmol), tris(dibenzylideneacetone)dipalladium(0) (3.7 mg, 0.004 mmol) and 4,5-Bis(diphenylphosphino)-9,9-dimethylxanthene (7.0 mg, 0.012 mmol) were combined in p-dioxane (1 ml) under nitrogen in a sealed vessel, purged with nitrogen and heated at about 100° C. for about 24 hours. The reaction was cooled, filtered through celite and then concentrated under reduced pressure. The crude mixture contained 4-(5-Pyridin-2-yl-thiophen-2-ylamino)-thieno[2,3-c]pyridine-2-carboxylic acid methyl ester and was used in the next step without further purification. RP-HPLC (Table 1, Method i) Rₜ=2.87 min; m/z: (M+H)⁻ 366.

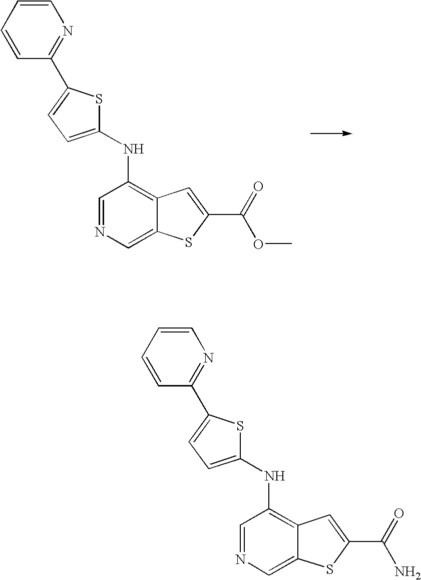

### Preparation #121: 4-(5-Pyridin-2-yl-thiophen-2-ylamino)-thieno[2,3-c]pyridine-2-carboxylic acid amide, Example 616

Following general procedure BB, crude 4-(5-Pyridin-2-yl-thiophen-2-ylamino)-thieno[2,3-c]pyridine-2-carboxylic acid methyl ester (15 mg, mmol) was charged into 7N methanolic ammonia (3 mL) and heated at about 60° C. for about 4 hours. The reaction was cooled to room temperature. RP-HPLC (Table 1, Method i) Rₜ=3.94 min; m/z: (M+H)⁺ 353.

### Preparation #122: [2-(5-Amino-[1,2,4]oxadiazol-3-yl)-thieno[2,3-c]pyridin-4-yl]-biphenyl-4-yl-amine, Example 617

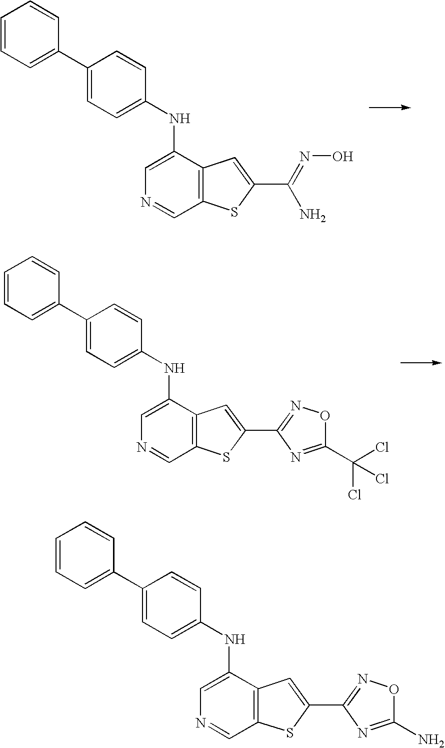

### a) Biphenyl-4-yl-[2-(5-trichloromethyl-[1,2,4]oxadiazol-3-yl)-thieno[2,3-c]pyridin-4-yl]-amine

Following general procedure ZZ, 4-(Biphenyl-4-ylamino)-N-hydroxy-thieno[2,3-c]pyridine-2-carboxamidine (0.2 g, 0.00055 mol) was suspended in anhydrous toluene (15 mL); the suspension was cooled to 0° C. and trichloroacetic anhydride was added dropwise. The mixture was refluxed for 2 hours. The reaction mixture was concentrated under reduced pressure to yield biphenyl-4-yl-[2-(5-trichloromethyl-[1,2,4]oxadiazol-3-yl)-thieno[2,3-c]pyridin-4-yl]-amine (0.27 g, 0.00055 mol) as a brown solid. Retention time—4.07 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

### b) [2-(5-Amino-[1,2,4]oxadiazol-3-yl)-thieno[2,3-c]pyridin-4-yl]-biphenyl-4-yl-amine

Following general procedure BB, Biphenyl-4-yl-[2-(5-trichloromethyl-[1,2,4]oxadiazol-3-yl)-thieno[2,3-c]pyridin-4-yl]-amine (0.17 g, 0.00035 mol) was digested with the saturated solution of ammonia in ethanol (7 mL) and the reaction mixture was heated in an autoclave at 100° C. for 2 hours. The solvent was removed under reduced pressure; the residue was triturated in DCM (10 mL) and the precipitate collected by filtration and dried. It was purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford [2-(5-amino-[1,2,4]oxadiazol-3-yl)-thieno[2,3-c]pyridin-4-yl]-biphenyl-4-yl-amine (0.067 g, 0.00017 mol) as a yellow solid.

Retention time—6.61 min., RP-HPLC (10% to 80% acetonitrile/0.01M aqueous ammonium acetate, buffered to pH 4.5, over 6 min at 0.8 mL/min; λ=190-700 nm; Genesis C18, 120 Å, 4 μm, 33×4.6 mm column.).

m/z: (M+H)⁺ 386.

### Preparation #123: Biphenyl-4-yl-[2-(5-isopropylamino-[1,2,4]oxadiazol-3-yl)-thieno[2,3-c]pyridin-4-yl]-amine, Example 618

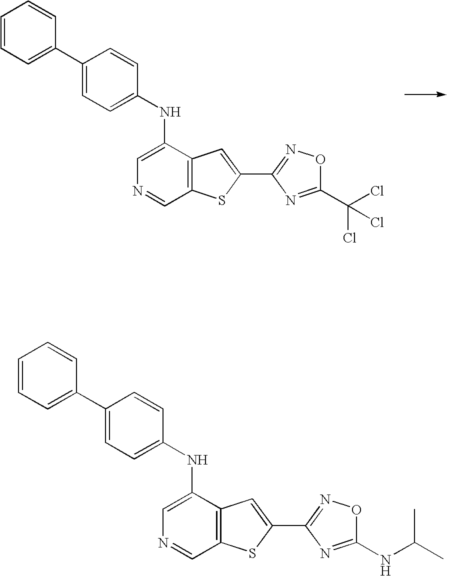

Following general procedure BB, biphenyl-4-yl-[2-(5-trichloromethyl-[1,2,4]oxadiazol-3-yl)-thieno[2,3-c]pyridin-4-yl]-amine (0.17 g, 0.00035 mol) was digested with the solution of isopropylamine (1 mL) in ethanol (7 mL) and the reaction mixture was heated in an autoclave at 100° C. for 2 hours. The solvent was removed under reduced pressure; the residue was triturated in DCM (10 mL) and the precipitate collected by filtration and dried. It was purified by preparative RP-HPLC (70% to 100% acetonitrile/0.05M aqueous ammonium acetate, buffered to pH 4.5, over 20 min at 21 mL/min; λ=254 nm; Microsorb C18, 100 Å, 5 μm, 250×46 mm column) to afford biphenyl-4-yl-[2-(5-isopropylamino-[1,2,4]oxadiazol-3-yl)-thieno[2,3-c]pyridin-4-yl]-amine (0.023 g, 0.000054 mol) as a yellow solid.

Retention time—3.09 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 428.

### Preparation #124: N-{3-[4-(Biphenyl-4-ylamino)-thieno[2,3-c]pyridin-2-yl]-[1,2,4]oxadiazol-5-yl}-2,2,2-trifluoroacetamide, Example 619

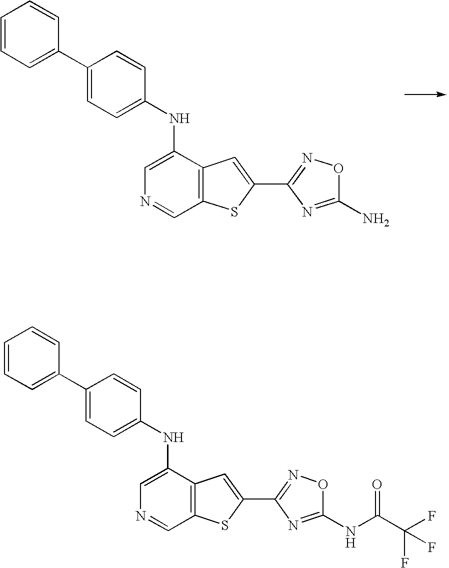

Following general procedure HH, [2-(5-Amino-[1,2,4]oxadiazol-3-yl)-thieno[2,3-c]pyridin-4-yl]-biphenyl-4-yl-amine (0.058 g, 0.00015 mol) was triturated in a mixture of anhydrous DCM (3 mL) and pyridine (0.75 mL); the suspension was cooled to 0° C. and trifluoroacetic anhydride (0.022 mL, 0.000159 mol) was added dropwise. The resulting mixture was stirred at ambient temperature under continuous nitrogen flow for 2 hours. The organic solvent was removed under reduced pressure and the residue purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford N-{3-[4-(biphenyl-4-ylamino)-thieno[2,3-c]pyridin-2-yl]-[1,2,4]oxadiazol-5-yl}-2,2,2-trifluoroacetamide (0.018 g, 0.000037 mol) as a yellow solid.

Retention time—6.77 min., RP-HPLC (10% to 80% acetonitrile/0.01M aqueous ammonium acetate, buffered to pH 4.5, over 6 min at 0.8 mL/min; λ=190-700 nm; Genesis C18, 120 Å, 4 μm, 33×4.6 mm column.).

m/z: (M+H)⁺ 482.

### Preparation #125: 4-(Biphenyl-4-ylamino)thieno[2,3-c]pyridine-2-carbohydrazonamide, Example 620

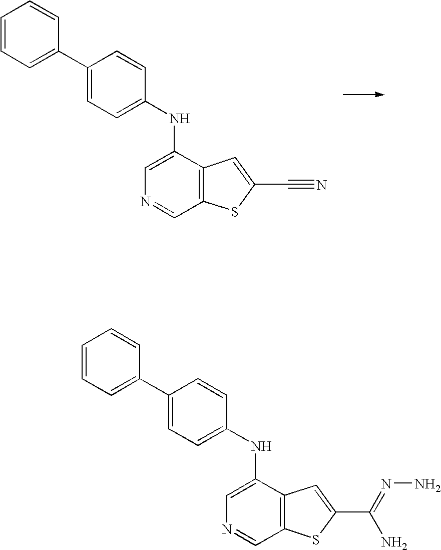

Following general procedure UU, 4-(Biphenyl-4-ylamino)-thieno[2,3-c]pyridine-2-carbonitrile (0.1 g, 0.00031 mol) and anhydrous hydrazine (0.096 mL, 0.0031 mol) were heated in anhydrous DMSO at 75° C. under continuous nitrogen flow for 18 hours. The solvent was removed under reduced pressure and the residue was purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford 4-(biphenyl-4-ylamino)thieno[2,3-c]pyridine-2-carbohydrazonamide (0.054 g, 0.00015 mol) as a yellow solid.

Retention time—1.69 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 358

### Preparation #126: Biphenyl-4-yl-[2-(4H-[1,2,4]triazol-3-yl)-thieno[2,3-c]pyridin-4-amine, Example 621

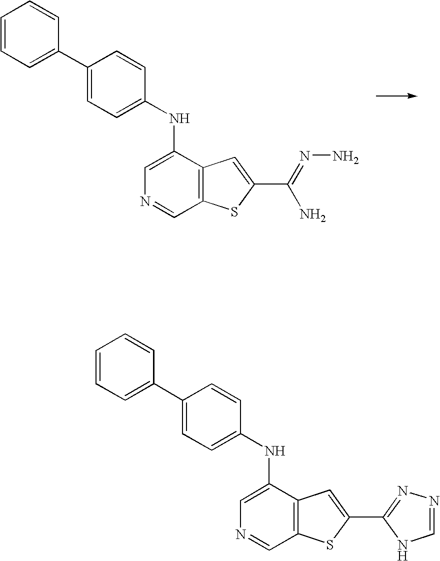

Following general procedure VV, 4-(Biphenyl-4-ylamino)thieno[2,3-c]pyridine-2-carbohydrazonamide (0.1 g, 0.00028 mol) was dissolved in triethyl orthoformate (5 mL), boron trifluoride diethyl ether (0.01 mL) was added and the reaction mixture was heated at reflux under continuous nitrogen flow for 2 hours. The solvent was removed under reduced pressure and the residue was purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford biphenyl-4-yl-[2-(4H-[1,2,4]triazol-3-yl)-thieno[2,3-c]pyridin-4-yl]-amine (0.007 g, 0.000019 mol) as a yellow solid.

Retention time—2.26 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 368

### Preparation #127: 5-[4-(Biphenyl-4-ylamino)-thieno[2,3-c]pyridin-2-yl]-2,4-dihydro-[1,2,4]triazol-3-one, Example 622

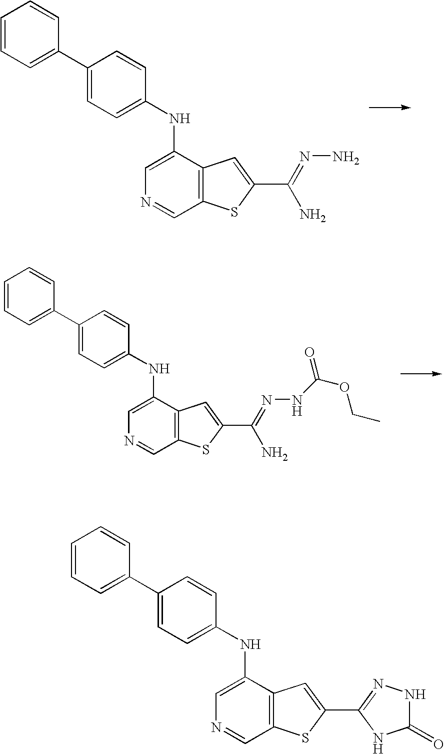

### a) N′-[1-Amino-1-[4-(biphenyl-4-ylamino)-thieno[2,3-c]pyridin-2-yl]-methyl-idene]-hydrazinecarboxylic acid ethyl ester hydrochloride

Following general procedure HH, 4-(Biphenyl-4-ylamino)thieno[2,3-c]pyridine-2-carbohydrazonamide (0.314 g, 0.00088 mol) was suspended in anhydrous ethanol (15 mL) and ethyl chloroformate (0.075 mL, 0.000787 mol) was added dropwise. The resulting mixture was stirred at ambient temperature under continuous nitrogen flow for 2 hours. The resulting precipitate was collected by filtration and dried to yield N′-[1-amino-1-[4-(biphenyl-4-ylamino)-thieno[2,3-]pyridin-2-yl]-methyl-idene]-hydrazinecarboxylic acid ethyl ester hydrochloride (0.344 g, 0.00074 mol) as a yellow solid.

Retention time—10.2 min., RP-HPLC (50% to 95% acetonitrile/0.01M aqueous ammonium acetate, buffered to pH 4.5, over 10 min at 1.7 mL/min; λ=254 nm; Hypersil C18, 100 Å, 5 μm, 250×4.6 mm column.).

### b) 5-[4-(Biphenyl-4-ylamino)-thieno[2,3-c]pyridin-2-yl]-2,4-dihydro-[1,2,4]triazol-3-one

Following general procedure VV, N′-[1-Amino-1-[4-(biphenyl-4-ylamino)-thieno[2,3-c]pyridin-2-yl]-methyl-idene]-hydrazinecarboxylic acid ethyl ester hydrochloride (0.1 g, 0.00021 mol) was suspended in a solution of potassium carbonate (0.9 g) in water (20 mL) and the reaction mixture was heated at reflux under continuous nitrogen flow for 8 hours. The precipitate was collected by filtration, dried and recrystallized from DMSO:MeOH=1:1 to yield 5-[4-(biphenyl-4-ylamino)-thieno[2,3-c]pyridin-2-yl]-2,4-dihydro-[1,2,4]triazol-3-one (0.04 g, 0.00010 mol) as a yellow solid.

Retention time—2.23 min., RP-HPLC (30% to 95% acetonitrile/0.01M aqueous ammonium acetate, buffered to pH 4.5, over 4.5 min at 0.8 mL/min; λ=190-700 nm; Genesis C 18, 120 Å, 4 μm, 33×4.6 mm column.).

m/z: (M−H)⁻ 384.

### Preparation #128: Biphenyl-4-yl-[2-(5-trifluoromethyl-4H-[1,2,4]triazol-3-yl)-thieno[2,3-c]pyridin-4-yl]-amine, Example 623

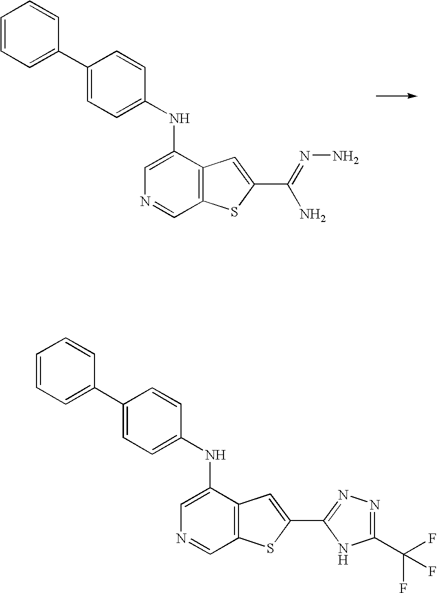

Following general procedure VV, to the ice-cold trifluoroacetic acid (7 mL), 4-(biphenyl-4-ylamino)thieno[2,3-c]pyridine-2-carbohydrazonamide (0.11 g, 0.00031 mol) was added and the resulting mixture was refluxed under continuous nitrogen flow for 1 hour. Trifluoroacetic acid was removed under reduced pressure; the residue was triturated in the saturated sodium bicarbonate solution in water (10 mL) and the precipitate was collected by filtration. It was purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford biphenyl-4-yl-[2-(5-trifluoromethyl-4H-[1,2,4]triazol-3-yl)-thieno[2,3-c]pyridin-4-yl]-amine (0.060 g, 0.00014 mol) as a yellow solid.

Retention time—2.24 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 436.

### Preparation #129: 4-[4-(Benzooxazol-2-ylamino)-phenyl]-thieno[2,3-c]pyridine-2-carboxylic acid amide, Example 624

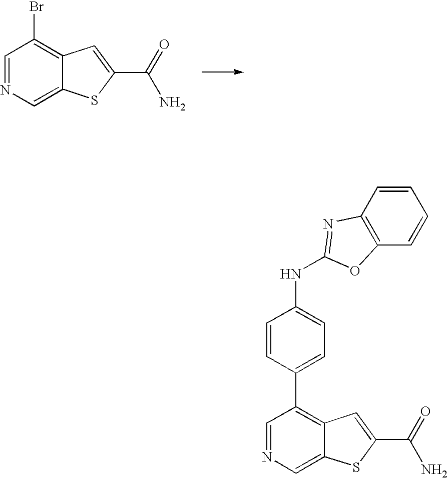

Following general procedure J, a mixture containing 4-bromo-thieno[2,3-c]pyridine-2-carboxylic acid amide (0.2 g, 0.00078 mol), benzooxazol-2-yl-[4-(4,4,5,5-tetramethyl-[1,3,2]dioxaborolan-2-yl)-phenyl]-amine (WO 02/076986) (0.288 g, 0.000856 mol), tris(dibenzylideneacetone) dipalladium(0) (0.018 g, 0.00002 mol), tri-t-butylphosphonium tetrafluoroborate ( 0.012 g, 0.000039 mol) and sodium carbonate (0.273 g, 0.0026 mol) in dioxane(6 mL) and water (3 mL) was stirred at ambient temperature for 18 hours. The solvents were removed under reduced pressure and the residue purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford 4-[4-(benzooxazol-2-ylamino)-phenyl]-thieno[2,3-c]pyridine-2-carboxylic acid amide (0.059 g, 0.00015 mol) as an off-white solid.

Retention time—2.33 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 385.

### Preparation #130: 4-[3-Fluoro-4-(5-fluoro-benzooxazol-2-ylamino)-phenyl]-thieno[2,3-c]pyridine-2-carboxylic acid amide, Example 625

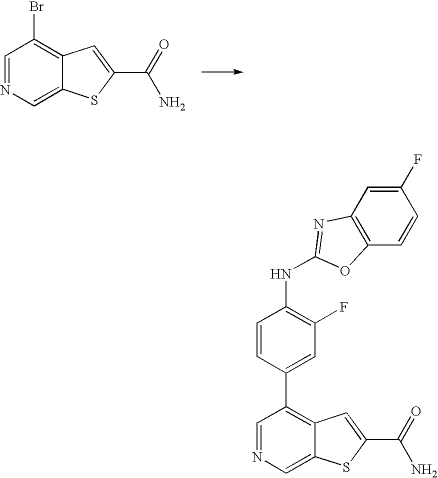

Following general procedure J, a mixture containing 4-bromo-thieno[2,3-c]pyridine-2-carboxylic acid amide (0.2 g, 0.00078 mol), (5-fluoro-benzooxazol-2-yl)-[2-fluoro-4-(4,4,5,5-tetramethyl-[1,3,2]dioxaborolan-2-yl)-phenyl]-amine (WO 02/076986) (0.319 g, 0.000856 mol), tris(dibenzylideneacetone) dipalladium(0) (0.018 g, 0.00002 mol), tri-t-butylphosphonium tetrafluoroborate (0.012 g, 0.000039 mol) and sodium carbonate (0.273 g, 0.0026 mol) in dioxane(6 mL) and water (3 mL) was stirred at ambient temperature for 18 hours. The solvents were removed under reduced pressure, the residue was triturated in water (25 mL) and the precipitate collected by filtration. It was recrystallized from DMF to afford 4-[3-fluoro-4-(5-fluoro-benzooxazol-2-ylamino)-phenyl]-thieno[2,3-c]pyridine-2-carboxylic acid amide (0.082 g, 0.00019 mol) as an off-white solid.

Retention time—2.49 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 421.

### Preparation #131: Biphenyl-4-yl-[2-(3H-[1,2,3]triazol-4-yl)-thieno[2,3-c]pyridin-4-yl]-amine, Example 626

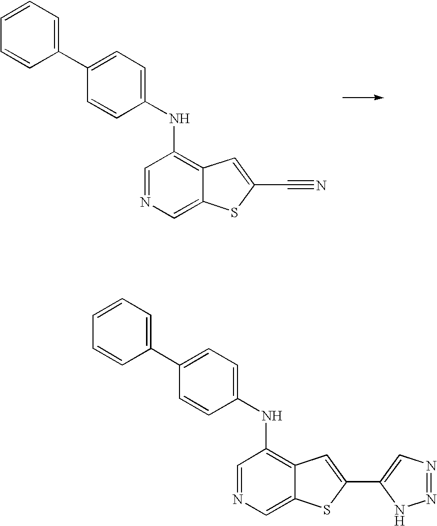

Following general procedure UU, to a 2 M solution of trimethylsilyl diazomethane in heptane (0.037 mL, 0.00073 mol) diluted with anhydrous THF (3 mL), a 2.5 M n-butyl lithium solution in heptane (0.029 mL, 0.00073 mol) was added at 0° C. and the resulting mixture was stirred at this temperature under continuous nitrogen flow for 20 min. To this mixture, the solution of 4-(biphenyl-4-ylamino)-thieno[2,3-c]pyridine-2-carbonitrile (0.2 g, 0.00061 mol) in anhydrous THF was added dropwise and the reaction mixture was stirred at 0° C. for 2 hours. It was quenched by a slow addition of a saturated solution of ammonium chloride in water (15 mL) and the organic phase was further extracted with EtOAc (2×25 mL). The combined organic extracts were concentrated and the residue purified by preparative RP-HPLC (30% to 60% acetonitrile/0.05M aqueous ammonium acetate, buffered to pH 4.5, over 30 min at 21 mL/min; λ=254 nm; Microsorb C18, 100 Å, 5 μm, 250×46 mm column) to afford biphenyl-4-yl-[2-(3H-[1,2,3]triazol-4-yl)-thieno[2,3-c]pyridin-4-yl]-amine (0.009 g, 0.000024 mol) as a yellow solid.

Retention time—2.76 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 370.

### Preparation #132: 3-[4-(Biphenyl-4-ylamino)-thieno[2,3-c]pyridin-2-yl]-3-oxo-propionitrile, Example 627

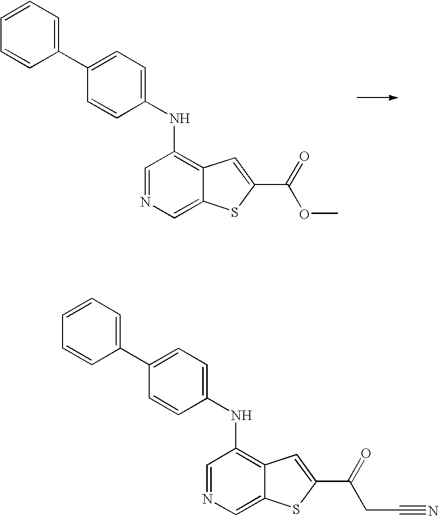

Following general procedure HH, to a solution of acetonitrile (0.087 mL, 0.0017 mol) in anhydrous DMF, 60% sodium hydride dispersion in parafines (0.07 g, 0.0018 mol) was added and the resulting mixture was stirred at ambient temperature under continuous nitrogen flow for 20 min. To the resulting mixture, a solution of 4-(biphenyl-4-ylamino)-thieno[2,3-c]pyridine-2-carboxylic acid methyl ester (0.3 g, 0.00084 mol) in anhydrous DMF (8 mL) was added dropwise and the stirring was continued for 3 hours at 65° C. The solvent was removed under reduced pressure and the residue purified by preparative RP-HPLC (40% to 85% acetonitrile/0.05M aqueous ammonium acetate, buffered to pH 4.5, over 30 min at 21 mL/min; λ=254 nm; Microsorb C18, 100 Å, 5 μm, 250×46 mm column) to afford 3-[4-(biphenyl-4-ylamino)-thieno[2,3-c]pyridin-2-yl-3-oxo-propionitrile (0.123 g, 0.00033 mol) as an orange solid.

Retention time—2.48 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 370.

### General procedure GGG: Addition of an isocyanate to an enolate

A mixture of a substituted 3-oxo-acetonitrile or a 3-oxo-acetate and an isocyanate (1-10 equivalents, preferably 1 equivalent) in an anhydrous solvent (CH₂Cl₂, DMF, dioxane, preferably DMF) was stirred at about 0-50° C. (preferably about 20° C.) for about 1-48 h (preferably about 3 h) at room temperature. The solvent was removed in vacuo to afford the product which can be further purified by chromatography or crystallization.

Illustration of General Procedure GGG

### Preparation #133: Triethylammonium 3-[4-(biphenyl-4-ylamino)-thieno[2,3-c]pyridin-2-yl]-2-cyano-3-oxo-N-phenyl-propionamide, Example 628

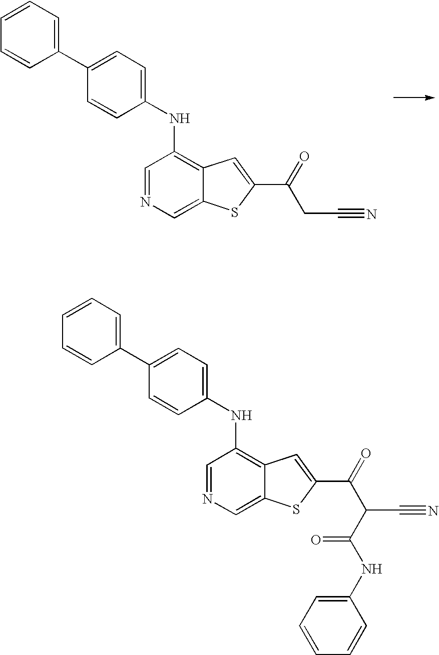

The mixture of 3-[4-(biphenyl-4-ylamino)-thieno[2,3-c]pyridin-2-yl]-3-oxo-propionitrile (0.055 g, 0.00015 mol) and phenyl isocyanate (0.016 mL, 0.00015 mol) in anhydrous DMF (2 mL) was stirred for 3 hours. The solvent was removed under reduced pressure; the residue was triturated in acetonitrile (10 mL) and the precipitate collected by filtration and dried to yield triethylammonium 3-[4-(biphenyl-4-ylamino)-thieno[2,3-c]pyridin-2-yl]-2-cyano-3-oxo-N-phenyl-propionamide (0.05 g, 0.000085 mol) as a yellow solid.

Retention time—2.47 min., RP-HPLC (30% to 95% acetonitrile/0.01M aqueous ammonium acetate, buffered to pH 4.5, over 4.5 min at 0.8 ml/min; λ=190-700 nm; Genesis C18, 120 Å, 4 μm, 33×4.6 mm column.).

m/z: (M−H)⁻ 487.

### Preparation #134: 3-[4-(Biphenyl-4-ylamino)-thieno[2,3-c]pyridin-2-yl]-2-cyano-N-isopropyl-3-oxo-propionamide, Example 629

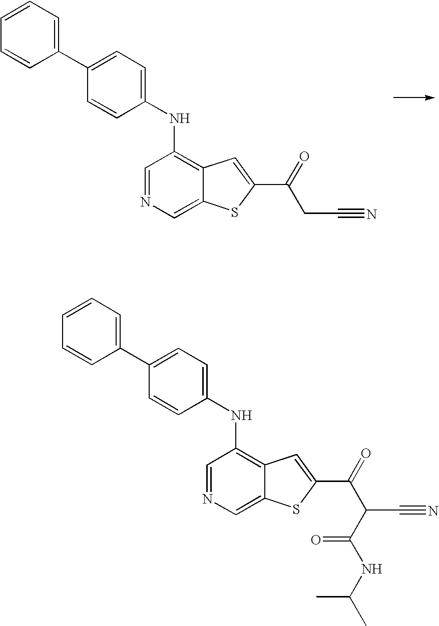

Following procedure GGG, the mixture of 3-[4-(biphenyl-4-ylamino)-thieno[2,3-c]pyridin-2-yl]-3-oxo-propionitrile (0.055 g, 0.00015 mol) and isopropyl isocyanate (0.015 mL, 0.00015 mol) in anhydrous DMF (2 mL) was stirred for 72 hours. The solvent was removed under reduced pressure and the residue purified by preparative RP-HPLC (40% to 80% acetonitrile/0.05M aqueous ammonium acetate, buffered to pH 4.5, over 20 min at 21 mL/min; λ=254 nm; Microsorb C18, 100 Å, 5 μm, 250×46 mm column) to afford 3-[4-(Biphenyl-4-ylamino)-thieno[2,3-c]pyridin-2-yl]-2-cyano-N-isopropyl-3-oxo-propionamide (0.017 g, 0.00037 mol) as a yellow solid.

Retention time—2.72 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 453.

### Preparation #135: 4-(Biphenyl-4-ylamino)-thieno[2,3-c]pyridine-2-carboxylic acid hydrazide, Example 630

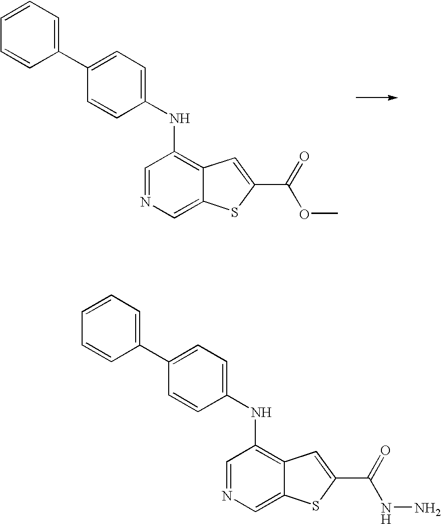

Following general procedure BB, the solution of 4-(biphenyl-4-ylamino)-thieno[2,3-c]pyridine-2-carboxylic acid methyl ester (0.5 g, 0.0014 mol) in hydrazine hydrate (7 mL) and ethanol (30 mL) was heated at reflux under continuous nitrogen flow for 1 hour. The solvents were removed under reduced pressure; the residue was triturated in water (40 mL) and the precipitate collected by filtration and dried to afford 4-(biphenyl-4-ylamino)-thieno[2,3-c]pyridine-2-carboxylic acid hydrazide (0.45 g, 0.0013 mol) as a yellow solid.

Retention time—2.29 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 361.

### Preparation #136: 5-[4-(biphenyl-4-ylamino)-thieno[2,3-c]pyridin-2-yl]-3H-[1,3,4]oxadiazol-2-one, Example 631

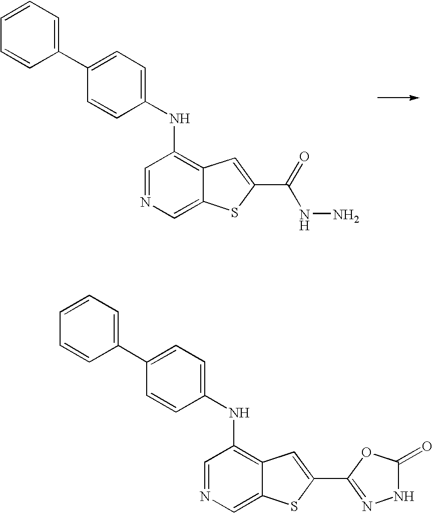

Following procedure VV, to the solution of triphosgene (0.099 g, 0.00033 mol) in dioxane (5 mL), the solution of 4-(biphenyl-4-ylamino)-thieno[2,3-c]pyridine-2-carboxylic acid hydrazide (0.12 g, 0.00033 mol) in dioxane (10 mL) was added dropwise at 0° C. under continuous nitrogen flow. Upon the completion of the addition, the solvent was removed under reduced pressure; the residue was suspended in saturated sodium bicarbonate solution I water (20 mL) and the precipitate collected by filtration and dried to yield 5-[4-(biphenyl-4-ylamino)-thieno[2,3-c]pyridin-2-yl]-3H-[1,3,4]oxadiazol-2-one (0.118 g, 0.00029 mol) as a yellow solid.

Retention time—2.70 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 385.

### Preparation #137: [2-(5-Amino-[1,3,4]oxadiazol-2-yl)-thieno[2,3-c]pyridin-4-yl]-biphenyl-4-yl-amine, Example 632

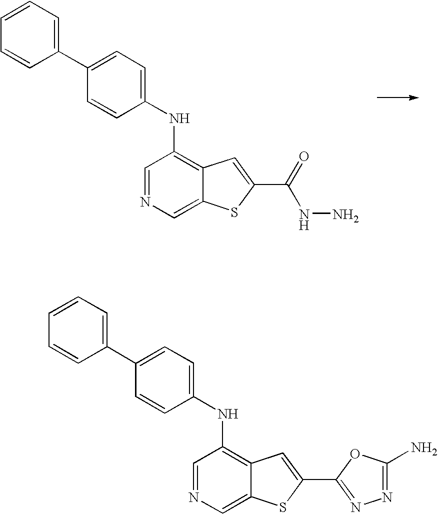

Following general procedure VV, to the suspension of 4-(biphenyl-4-ylamino)-thieno[2,3-c]pyridine-2-carboxylic acid hydrazide (0.25 g, 0.00069 mol) in dioxane (10 mL), cyanogens bromide (0.074 g, 0.00069 mol) was added followed by the addition of the solution of sodium bicarbonate (0.058 g, 0.000694 mol) in water (10 mL). The reaction mixture was stirred at ambient temperature under continuous nitrogen flow for 16 hours. The solvents were removed under reduced pressure and the residue purified by preparative RP-HPLC (30% to 60% acetonitrile/0.05M aqueous ammonium acetate, buffered to pH 4.5, over 30 min at 21 mL/min; λ=254 nm; Microsorb C18, 100 Å, 5 μm, 250×46 mm column) to afford [2-(5-amino-[1,3,4]oxadiazol-2-yl)-thieno[2,3-c]pyridin-4-yl]-biphenyl-4-yl-amine (0.006 g, 0.000015 mol) as a yellow solid.

Retention time—2.57 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 386.

### Preparation #138: Acetic acid [1-amino-1-[4-(biphenyl-4-ylamino)-thieno[2,3-c]pyridin-2-yl]-methylidene]-hydrazide, Example 633

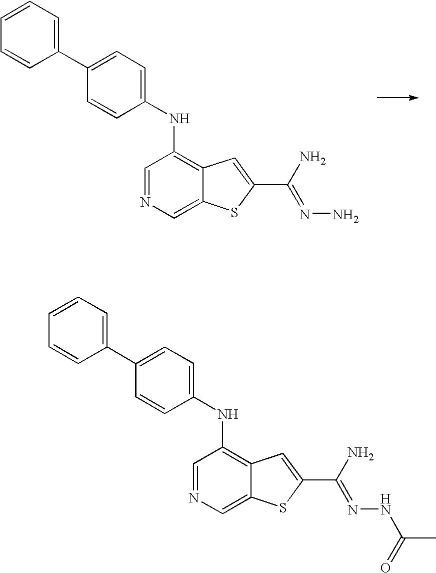

Following general procedure HH, 4-(Biphenyl-4-ylamino)thieno[2,3-c]pyridine-2-carbohydrazonamide (0.19 g, 0.00054 mol) was suspended in DCM (4 mL) and to the resulting mixture, acetic anhydride (0.035 mL, 0.00037 mol) was added dropwise. The reaction mixture was stirred at ambient temperature for 1 hour under continuous nitrogen flow. The solvent was removed under reduced pressure and the residue purified by preparative RP-HPLC (30% to 60% acetonitrile/0.05M aqueous ammonium acetate, buffered to pH 4.5, over 30 min at 21 mL/min; λ=254 nm; Microsorb C18, 100 Å, 5 μm, 250×46 mm column) to afford acetic acid [1-amino-1-[4-(biphenyl-4-ylamino)-thieno[2,3-c]pyridin-2-yl]-methylidene]-hydrazide (0.093 g, 0.00023 mol) as a yellow solid.

Retention time—2.27 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 Åm, 33×4.6 mm column.).

m/z: (M−H)⁻ 400.

### General procedure HHH: Formation of a 3-aminopyrazole

A mixture of a substituted 3-oxo-acetonitrile and hydrazine hydrate (1-20 equivalents, preferably 5 equivalents) in an organic solvent (ethanol, methanol, acetic acid, preferably ethanol) was heated at about 30-100° C. (preferably about 78° C.) for about 1-48 h (preferably about 10 h) under a nitrogen atmosphere. The solvent was removed in vacuo to afford the product which can be further purified by chromatography or crystallization.

Illustration of General Procedure HHH

### Preparation #139. [2-(5-Amino-2H-pyrazol-3-yl)-thieno[2,3-c]pyridin-4-yl]-biphenyl-4-yl-amine, Example 634

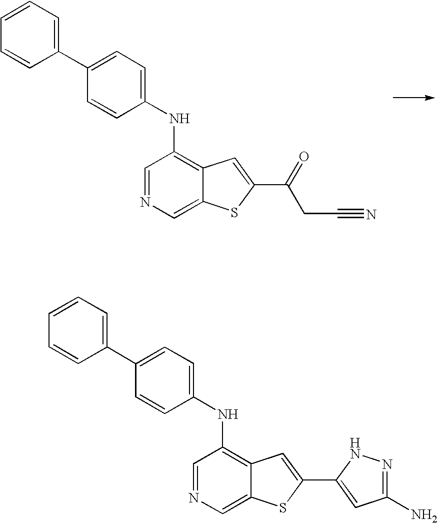

The mixture of 3-[4-(biphenyl-4-ylamino)-thieno[2,3-c]pyridin-2-yl]-3-oxo-propionitrile (0.07 g, 0.00019 mol) and hydrazine hydrate (0.3 mL) in ethanol (5 mL) was refluxed for 10 hours under continuous nitrogen flow. The solvent was removed under reduced pressure and the residue purified by preparative RP-HPLC (40% to 70% acetonitrile/0.05M aqueous ammonium acetate, buffered to pH 4.5, over 30 min at 21 mL/min; λ=254 nm; Microsorb C18, 100 Å, 5 μm, 250×46 mm column) to afford [2-(5-Amino-2H-pyrazol-3-yl)-thieno[2,3-c]pyridin-4-yl]-biphenyl-4-yl-amine (0.023 g, 0.00006 mol) as a yellow solid.

Retention time—2.29 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 384.

### General procedure III: Reduction of carboxylic acid

A carboxylic ester (preferably one equivalent) is dissolved in an organic solvent (preferably ethanol) and treated with CaCl2 (preferably two equivalents). Co-solvents (preferably THF) may be added to aid solubility. The mixture is stirred for 1-4 hours (preferably 1 hour) at room temperature and the reaction mixture is cooled to 0° C. Excess Sodium cyanoborohydride (preferably 4 equivalents) is added in portions. The mixture is stirred for about ½-8 hour at 0° C. and then allowed to warm to room temperature with stirring for 1-24 hours. The mixture is treated with water and extracted with CH₂CL₂. The extract was washed with brine, dried (MgSO4), filtered and concentrated. The residue can be further purified by flash chromatography on silica gel.

Illustration of General Procedure III

### Preparation #140: (4-Bromo-thieno[2,3-c]pyridin-2-yl)-methanol

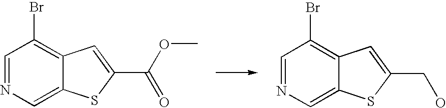

4-Bromo-thieno[2,3-c]pyridine-2-carboxylic acid methyl (prepared using general procedures A and B) (1.09 g, 4.00 mmol) was suspended in ethanol (20.0 mL) and treated with CaCl₂ (888 mg, 8.00 mmol). Tetrahydrofuran (10 ml) was added to aid solubility and the mixture was stirred for 1 hour at room temperature. The reaction mixture was cooled to 0° C. and Sodium cyanoborohydride (608 mg, 16.0 mmol)) was added in portions. The mixture is stirred for 1 hour at 0° C. and then allowed to warm to room temperature with stirring overnight. The mixture is treated with water (25 ml) and extracted with CH₂CL₂ (40 ml). The extract was washed with brine (25 ml), dried (MgSO4), filtered and concentrated to yield 705 mg (72%) of (4-Bromo-thieno[2,3-c]pyridin-2-yl)-metlhanol as a white solid; RP-HPLC (Table 1, Method M, R. 1.27 min; m/z: (M+H)⁺ 244/246.

### General procedure JJJ: Epoxide ring opening with N-hydroxyphthalimide

A substituted epoxide (1-6 equivalents, preferably 3.3 equivalents), Hydroxyphthalimide (preferably one equivalent) and triethylamine (1-4 equivalents, preferably one equivalent) are combined in an organic solvent (preferably DMF) and the mixture is heated (preferably by microwave) to 150° C. for 20 min. The mixture is concentrated under reduced pressure and the residue is purified by flash chromatography on silica gel.

Illustration of General Procedure JJJ

### Preparation #141: 2-(2-Hydroxy-propoxy)-isoindole-1,3-dione

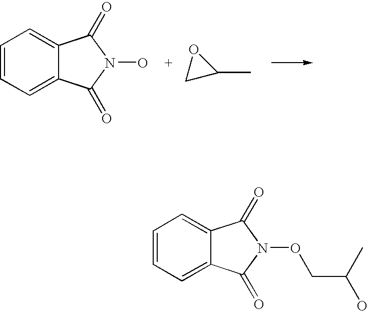

Propylene oxide (230 uL, 3.30 mmol), Hydroxyphthalimide (163 mg, 1.00 mmol) and triethylamine (139 uL, 1.00 mmol) are combined in an DMF (2.0 ml) and the mixture is heated by microwave at 150° C. for 20 min. The mixture is concentrated under reduced pressure and the residue is purified by flash chromatography on silica gel using 1:1/heptane:EtOAc as eluant. Product fractions are combined and concentrated to an oil which crystallizes to yield 219 mg (50%) of 2-(2-Hydroxy-propoxy)-isoindole-1,3-dione as a white solid; RP-HPLC (Table 1, Method 1), Rₜ 1.54 min; m/z: (M+H)⁺ 222.

### General procedure KKK: Conversion of Thieno[2,3c]pyridine N-oxide to 7-Oxo-Thieno[2,3-c]pyridine with optional ester hydrolysis

To a solution of a Thieno[2,3-c]pyridine N-oxide (preferably 1 eqivalent) in an organic solvent (preferably acetonitrile) is added water (preferably about 5% by volume) and 1-10 equivalents of 4-Methyl-benzenesulfonyl chloride (preferably about 4 equivalents) in portions. The reaction is heated at 30-80° C. (preferably 60° C.) and monitored periodically. When conversion is complete, the product may be isolated by extractive workup, preparative chromatography or crystallization.

In some cases, the product contains an ester which may be optionally hydrolysed with aqueous NaOH solutions (preferably 1-2 M solutions) as part of the work-up.

Illustration of General Procedure KKK

### Preparation #142: 4-Bromo-7-oxo-6,7-dihydro-thieno[2,3-c]pyridine-2-carboxylic acid methyl ester

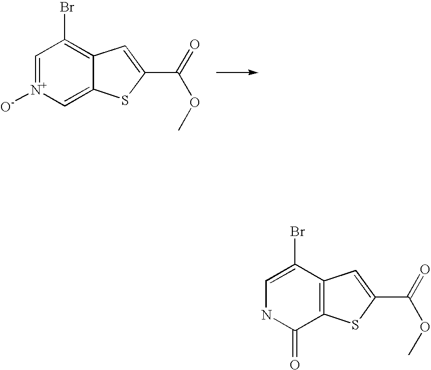

To a solution of a 4-Bromo-6-oxy-thieno[2,3-c]pyridine-2-carboxylic acid methyl ester (100 mg, 0.35 mmol) in acetonitrile (20 ml) was added water (1.0 ml) and Methyl-benzenesulfonyl chloride (42.0 mg, 0.22 mmol). The reaction was heated at 60° C. and monitored periodically. Additional portions of Methyl-benzenesulfonyl chloride (42.0 mg, 0.22 mmol) were added at ½ hour intervals and the reaction was continued overnight. On cooling, the product crystallized out of solution and was filtered off. A second crop was obtained on concentration of the mother liquors. The combined yield was 45 mg (45%) of 4-Bromo-7-oxo-6,7-dihydro-thieno[2,3-c]pyridine-2-carboxylic acid methyl ester as a white solid; RP-HPLC (Table 1, Method I), Rₜ 1.94 min; m/z: (M−H)⁻ 286/288.

Illustration of General Procedure KKK

### Preparation #143: 4-Biphenyl-3-yl-7-oxo-6,7-dihydro-thieno[2,3-c]pyridine-2-carboxylic acid

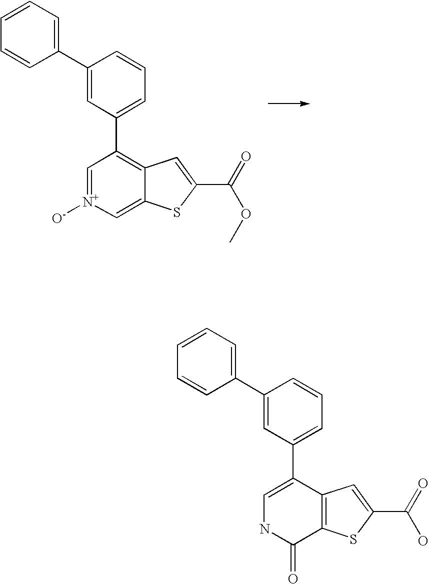

To a solution of a 4-Biphenyl-6-oxy-thieno[2,3-c]pyridine-2-carboxylic acid methyl ester (72.2 mg, 0.20 mmol) in p-dioxane (2 ml) and acetonitrile (12 ml) was added water (1.0 ml) and Methyl-benzenesulfonyl chloride (38.0 mg, 0.20 mmol). The reaction was heated at 60° C. and monitored periodically. Three additional portions of Methyl-benzenesulfonyl chloride (38.0 mg, 0.20 mmol) were added at ½ hour intervals and the reaction was continued for 5 hours. Aqueous NaOH (2N, 3.0 ml) was added and the solution was refluxed for one hour. The reaction was cooled and concentrated and the residue was further purified by preparative chromatography (method k) to yield 4-Biphenyl-3-yl-7-oxo-6,7-dihydro-thieno[2,3-c]pyridine-2-carboxylic acid product crystallized out of solution and was filtered off. A second crop was obtained on (23 mg, 33%) as an off-white solid; RP-HPLC (Table 1, Method I), Rₜ 1.65 min; m/z: (M−H)⁻ 346.

### General Procedure LLL: Reductive alkylation of an amine with and aldehyde followed by de-methylation of aromatic methoxy groups

The reductive alkylation is carried out according to General Procedure W. The crude product containing an aromatic methoxy group is treated with Boron tribromide in an organic solvent (preferably dichloromethane or heptane) at about room temperature for 1-6 hours. The reaction is cooled and quenched by addition of excess alcohol (preferably methanol). The products may be isolated via an extractive workup or the solvents may be removed and the crude product purified by preparative chromatography.

Illustration of General Procedure LLL

### Preparation #144: 4-[1-(2-Hydroxy-benzyl)-piperidin-4-ylamino]-thieno[2,3-c]pyridine-2-carboxylic acid amide

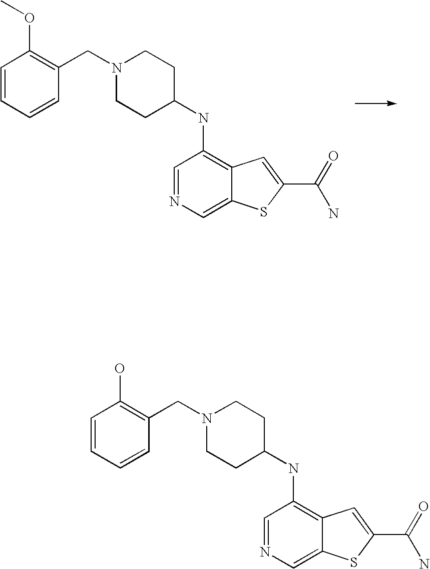

Crude 4-[1-(2-Methoxy-benzyl)-piperidin-4-ylamino]-thieno[2,3-c]pyridine-2-carboxylic acid amide (0.081 mmol) (from reductive alkylation with 2-Methoxy-benzaldehyde according to General Procedure W) was treated with 1M BBr₃CH₂CL₂ (0.5 ml) for 1 hour at room temperature under nitrogen. The reaction was cooled to 0° C. and quenched by addition of methanol (3 ml). Solvents were remove under reduced pressure and the crude product was purified by reverse phase preparative chromatography (method k) to yield 4-[1-(2-Hydroxy-benzyl)-piperidin-4-ylamino]-thieno[2,3-c]pyridine-2-carboxylic acid amide 13 mg (42%) as a yellow solid; RP-HPLC (Table 1, Method M), Rₜ 1.46 min; m/z: (M+H)⁺ 383.

### General Procedure MMM: Protection of an amine with a Cbz group

A primary or secondary amine salt (preferably 1 equivalent) is dissolved in water and treated with an inorganic base (preferably K₂CO₃) (preferably 1 equivalent). A solution of Carbonic acid benzyl ester 2,5-dioxo-pyrrolidin-1-yl ester (Cbz-OSu, preferably slightly less than 1 equivalent) in an organic solvent (preferably acetonitrile) is added at about room temperature and the reaction is allowed to proceed through completion. The mixture is concentrated to mostly water and the product is extracted into an organic solvent (preferably EtOAc), washed with aqueous acid and aqueous base, dried, filtered and concentrated.

Illustration of General Procedure MMM

### Preparation #145: 4-Oxo-piperidine-1-carboxylic acid benzyl ester

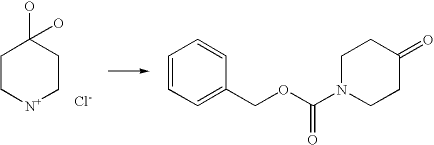

4-Piperidone monohydrate hydrochloride (5.0 g, 32.6 mmol) is dissolved in water (30 ml) and treated with K₂CO₃ (4.55 g, 33 mmol). A solution of Carbonic acid benzyl ester 2,5-dioxo-pyrrolidin-1-yl ester (Cbz-OSu, 7.0 g, 28.1 mmol) in acetonitrile (50 ml) is added at room temperature and the reaction is allowed to stir overnight. The mixture is concentrated to mostly water and the product is extracted into EtOAc (50 ml), washed with 5% aqueous citric acid (50 ml), saturated NaHCO₃ solution (50 ml) and brine (50 ml), dried (MgSO4), filtered and concentrated to yield 4-Oxo-piperidine-1-carboxylic acid benzyl ester 6.26 g (95%) as a clear, colorless oil; RP-HPLC (Table 1, Method M), Rₜ 1.64 min; m/z: (M+H)⁺ 234.

### General Procedure NNN: Wittig olefination reaction

A suspension of Methyltriphenylphosphonium bromide (preferably 2.5 equivalents) in an organic solvent (preferably THF) is cooled to about −78° C. and treated with n-BuLi/hexanes (preferably 2.2 equivalents) dropwise maintaining the pot temperature near −78° C. The mixture is warmed to room temperature for about 1 hour, and then cooled again to −70° to −78° C. A solution of ketone (preferably one equivalent) in organic solvent (preferably THF) is added dropwise and the solution is allowed to warm to room temperature. When complete, the reaction is diluted with an organic solvent (preferably EtOAc) and washed with aqueous solutions, dried, filtered and concentrated. The crude product may be further purified by crystallization or flash chromatography on silica gel.

Illustration of General Procedure NNN

### Preparation #146: 4-Methylene-piperidine-1-carboxylic acid benzyl ester

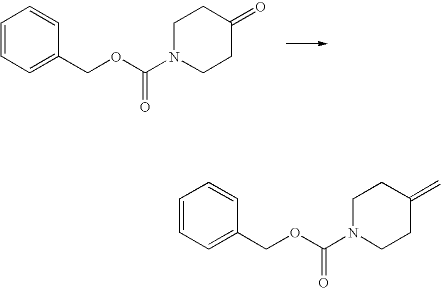

A suspension of Methyltriphenylphosphonium bromide (8.92 g, 25.0 mmol) in THF (200 ml) is cooled to −78° C. and treated with 2.5N n-BuLi/hexanes (8.8 ml, 22.0 mmol) dropwise maintaining the pot temperature below −74° C. The mixture is warmed to room temperature for about 1 hour, and then cooled again to −70° C. A solution of ketone (2.33 g, 10.0 mmol) in THF (10 ml) is added dropwise and the solution is allowed to warm to room temperature with stirring overnight. The reaction is diluted with EtOAc (50 ml) and washed with water (2×200 ml) and brine (50 ml), dried (MgSO4), filtered and concentrated. The crude product was further purified by flash chromatography on silica gel using 80:20/heptane:EtOAc as eluant. Product fractions were combined and concentrated to yield 4-Methylene-piperidine-1-carboxylic acid benzyl ester as a clear, colorless oil. (1.75 g, 75%): RP-HPLC (Table 1, Method M), Rₜ=4.02 min.; ¹H NMR (d₆-DMSO, 400 MHz): δ7.29-7.41 (5H, m), 5.09 (2H, s), 4.77 (2H, s), 3.36-3.46 (4H, t), 2.13-2.16 (4H, t)

### General procedure OOO: Suzuki coupling with in situ generation of borane

An olefin (preferably 1 equivalent) is dissolved in a solution of 9-Bora-bicyclo[3.3.1]nonane (9-BBN, preferably one equivalent) in an organic solvent (preferably THF) and heated at about 70° C. for 1-4 hours. After cooling, a catalyst such as Pd(PPh₃)₄ or Pd Cl₂ dppf (preferably 2-20 mol %), an inorganic base such as K₂CO₃ or Cs₂CO₃ (preferably 1-3 equivalents) and a solution of aryl halide (preferably about 1 equivalent) in an organic solvent such as DMF, THF or p-Dioxane are added. The mixture is de-gassed and heated at 50-100° C. for 1-24 hours. The mixture is cooled, filtered through silica gel and concentrated. Further purification can be achieved by flash chromatography on silica gel.

Illustration of General Procedure OOO

### Preparation #147: 4-(1-Benzyloxycarbonyl-piperidin-4-ylmethyl)-7-chloro-thieno[2,3-c]pyridine-2-carboxylic acid methyl ester

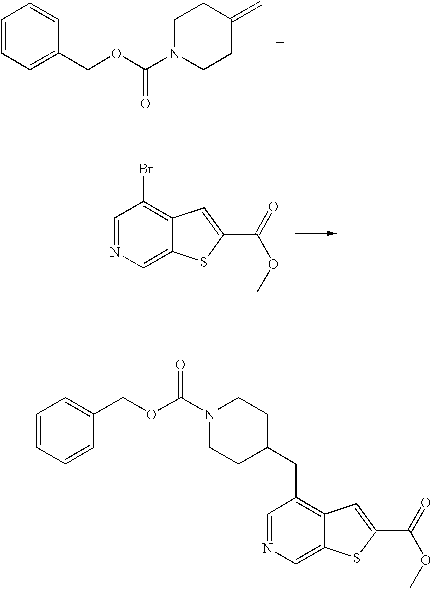

4-Methylene-piperidine-1-carboxylic acid benzyl ester (250 mg, 1.08 mmol) is dissolved in a 0.5 M solution of 9-Bora-bicyclo[3.3.1]nonane in THF (2.16 ml, 1.08 mmol) and heated at 67° C. for 1.5 hour. The mixture was cooled and Pd C1₂ dppf (40.8 mg, 0.05 mMol), Cs₂CO₃ (0.98 g, 3.00 mmol) and a solution 4-Bromo-thieno[2,3-c]pyridine-2-carboxylic acid methyl ester (272 mg, 1.00 mmol) in p-Dioxane (3.0 ml) were added. The mixture was de-gassed and heated at 90° C. for 3 hours. The mixture was cooled and filtered through silica gel chasing with EtOAc. The organic layer was concentrated and the residue was further purified by flash chromatography on silica gel using 4:1/CH₂CL₂:EtOAc, then 1:1/CH₂CL₂:EtOAc as eluant. Product fractions were combined and concentrated to yield 128 mg (30%) of 4-(1-Benzyloxycarbonyl-piperidin-4-ylmethyl)-thieno[2,3-c]pyridine-2-carboxylic acid methyl ester as a yellow solid; RP-HPLC (Table 1, Method M), Rₜ 2.40 min; m/z: (M+H)⁺ 425.

### General Procedure PPP: Stifle coupling to aromatic halide

An aromatic bromide (preferably 1 equivalent) is combined with a substituted tin reagent (preferably I equivalent) and a palladium catalyst such as Pd(PPh₃)₄ or Pd Cl₂ dppf (preferably 2-20 mol %) in an organic solvent such as THF or p-Dioxane. The mixture is degassed and heated at 80°-150° C. for 1-24 hours and then cooled. The reaction is filtered through silica gel, chasing with EtOAc and then the organic solvents are removed under reduced pressure. The residue can be further purified by preparative chromatography on silica gel or reversed phase columns.

Illustration of General Procedure PPP

### Preparation #148: 4-Vinyl-thieno[2,3-c]pyridine-2-carboxylic acid methyl ester

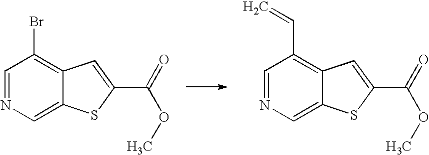

4-Bromo-thieno[2,3-c]pyridine-2-carboxylic acid methyl ester (272 mg, 1.0 mmol) was combined with Tributyl-vinyl-stannane (317 mg, 1.0 mmol) and Pd Cl₂dppf (40.8 mg, 0.05 mmol) in p-Dioxane (5.0 ml). The mixture is degassed and heated in a sealed vessel at 150° C. for 3 hours and then cooled. The reaction is filtered through silica gel, chasing with EtOAc and then the organic solvents are removed under reduced pressure. The residue was be further purified by preparative reversed phase chromatography (method k) to yield 4-Vinyl-thieno[2,3-c]pyridine-2-carboxylic acid methyl ester (109 mg, 50%) as an off-white solid; RP-HPLC (Table 1, Method M, Rₜ 1.96 min; m/z: (M+H)⁺ 220.

### General Procedure QQQ: Permanganate oxidation of an aromatic vinyl group

Potassium permanganate (1-10 equivalents, preferably about 5 equivalents) is combined with Al₂O₃ (preferably about o.6 g per 1 mmol KMnO₄) and water (preferably equal weight as KMnO₄) and the mixture is ground to homogeneity. A substrate containing an aromatic vinyl group (preferably 1 equivalent) in an organic solvent such as CH₂CL₂ is added and the mixture is refluxed for 1 to 24 hours. The reaction is allowed to cool and poured onto a pad of celite. The product is eluted with methanol and concentrated. The crude product may be further purified by preparative chromatography on silica gel or reversed phase columns.

Illustration of General Procedure QQQ

### Preparation #149: 4-Vinyl-thieno[2,3-c]pyridine-2-carboxylic acid methyl ester

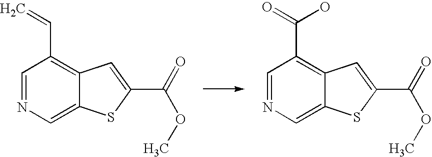

Potassium permanganate (0.78 g, 4.94 mmol) was combined with Al₂O₃ (3.12 g) and water (0.78 g) and the mixture was ground to homogeneity. 4-Vinyl-thieno[2,3-c]pyridine-2-carboxylic acid methyl ester (219 mg, 1.0 mmol) in CH₂CL₂ (35 ml) was added and the mixture was refluxed for 8 hours. The reaction was allowed to cool and poured onto a pad of celite. The product was eluted with methanol and concentrated. The crude product was further purified by preparative chromatography (method tg) to yield Thieno[2,3-c]pyridine-2,4-dicarboxylic acid 2-methyl ester (25.0 mg, 10%) as an off-white solid; RP-HPLC (Table 1, Method M, Rₜ 0.36 min; m/z: (M−H)⁻ 236.

### General Procedure RRR: Hydrolysis of Imine

A compound containing an imine group (preferably 1 equivalent) is dissolved in an organic solvent such as THF, p-Dioxane or DMF and dilute (preferably 1-2N solutions) aqueous acid (preferably HCl) is added at room temperature. The reaction is stirred at room temperature for 0.5 to 8 hours (preferably about 1 hour). The solvents are removed under reduced pressure and the residue may be further purified by trituration, crystallization or preparative chromatography of silica gel or reversed phase columns.

Illustration of General Procedure RRR

### Preparation #150: 7-Amino-4-bromo-thieno[2,3-c]pyridine-2-carboxylic acid methyl ester

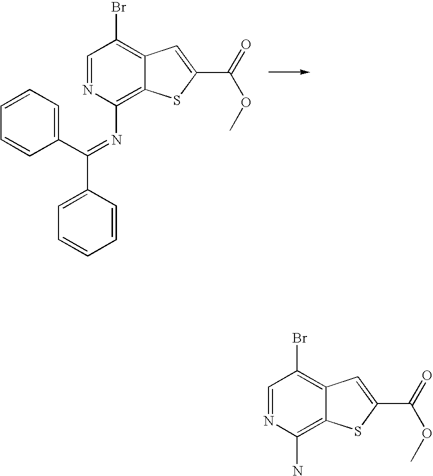

Crude 7-(Benzhydrylidene-amino)-4-bromo-thieno[2,3-c]pyridine-2-carboxylic acid methyl ester (15.7 mmol)) is dissolved in p-Dioxane (30 ml) and 2N HCl (25 ml) is added at room temperature. The reaction is stirred at room temperature for one hour and the precipitated product is filtered off. The solid is stirred with saturated NaHCO₃ for 30 min., filtered and washed with water and ether to yield 7-Amino-4-bromo-thieno[2,3-c]pyridine-2-carboxylic acid methyl ester (2.87 g, 69%) as a yellow solid; RP-HPLC (Table 1, Method M), Rₜ min; m/z: (M−H)⁻.

### General Procedure SSS: Amide formation with subsequent deprotection

Amide formation is carried out as described is General Procedure BBB with one or both components of the coupling containing acid labile protection groups. The crude product is then immediately treated with Trifluoroacetic acid (preferably 1-10 ml per mmol) with, or without a suitable cation scavenger such as water, anisole, or silanes for 5-120 minutes (preferably 30 minutes). Solvent is removed under reduced pressure and the crude product is further purified by column chromatography. Alternatively, the Tfa solution may be diluted with ether to precipitate the crude product that may be further purified by column chromatography.

Illustration of General Procedure SSS

### Preparation #151: 4-(4-Bromo-phenylamino)-thieno[2,3-c]pyridine-2-carboxylic acid (2-hydroxy-1-methyl-ethyl)amide

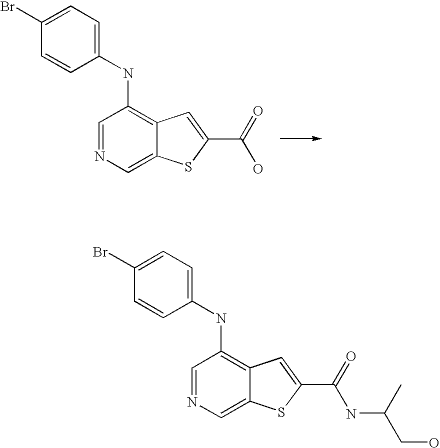

Amide formation is carried out as described is General Procedure BBB, using 4-(4-Bromo-phenylamino)-thieno[2,3-c]pyridine-2-carboxylic acid ( 65.0 mg, 0.186 mmol) and O-(2-tert-Butoxy-1-methyl-ethyl)-hydroxylamine (83.1 mg, 0.30 mmol). The crude product is then immediately treated with Trifluoroacetic acid (2 ml) containing triisopropylsilane (61.0 ul, 0.30 mmol) for 20 minutes at room temperature. Solvent is removed under reduced pressure and the crude product is further purified by column chromatography (method k) to yield 4-(4-Bromo-phenylamino)-thieno[2,3-c]pyridine-2-carboxylic acid (2-hydroxy-1-methyl-ethyl)amide (42 mg, 54%) as a yellow solid; RP-HPLC (Table 1, Method i), Rₜ 2.14 min; m/z: (M+H)⁺ 422/424.

### General Procedure TTT: Amide formation with subsequent nucleophilic displacement of an ester

Amide formation is carried out as described is General Procedure BBB with one or both components of the coupling containing ester groups. The crude product is then immediately treated with an amine according to General Procedure BB to convert the ester group to the corresponding amide. Solvent is removed under reduced pressure and the crude product is further purified by column chromatography or crystalization.

Illustration of General Procedure TTT

### Preparation #152: 4-(4-Benzyl-piperazine-1-carbonyl)-thieno[2,3-c]pyridine-2-carboxylic acid amide

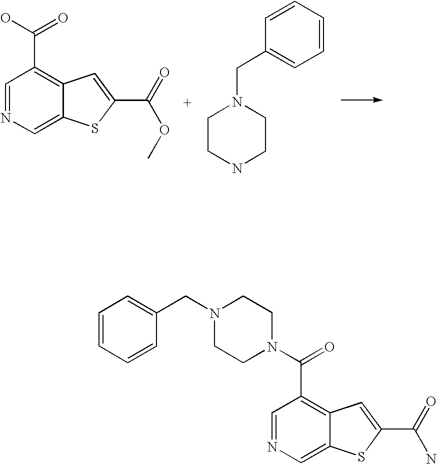

Amide formation is carried out by converting Thieno[2,3-c]pyridine-2,4-dicarboxylic acid 2-methyl ester (23 mg, 0.10 mmol) to it's corresponding acid chloride and performing a coupling to 1-Benzyl-piperazine (35 mg, 0.2 mmol) as described is General Procedure BBB. The crude product is then immediately treated with a 7M solution of ammonia in methanol (3 ml) in a sealed vessel at 100° C. for 1 hour as described in General Procedure BB to convert the ester group to the corresponding amide. Solvent is removed under reduced pressure and the crude product is further purified by column chromatography (method k) to yield 4-(4-Benzyl-piperazine-1-carbonyl)-thieno[2,3-c]pyridine-2-carboxylic acid amide (22 mg, 58%) as a yellow solid; RP-HPLC (Table 1, Method i), Rₜ 1.21 min; m/z: (M+H)⁺ 381.

### General Procedure UUU: Boronation reaction of an aryl bromide.

A mixture of an aryl bromide (preferably 1 equivalent), a diboron pinacol ester (1-2 equivalents, preferably 1 equivalents), an inorganic base (preferably potassium acetate) (1-4 equivalents, preferably 3 equivalents), and a palladiumcatalyst (preferably 1,1′-bis(diphenylphosphino)ferrocene-palladium dichloride) (0.05-0.2 equivalents, preferably 0.05 equivalents) is suspended in an anhydrous solvent (preferably N,N-dimethylformamide). The reaction is heated at about 80-100° C. (preferably about 80° C.) for about 1-24 hours (preferably about 12 hours).

The resulting mixture is allowed to cool to ambient temperature and filtered through a celite pad. The organic layer is subjected to further purification by crystallization or chromatography.

Illustration of General Procedure UUU

### Preparation #153: 3-(4′-cyano)biphenylboronic acid pinacol ester

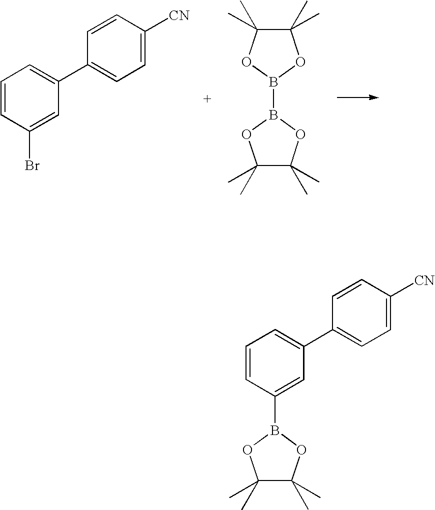

A mixture of 3′-bromo-biphenyl-4-carbonitrile (0.312 g, 1.02 mmol), diboron pinacol ester (0.272 g, 1.07 mmol), potassium acetate (0.300 g, 3.06 mmol) and 1,1′-bis(diphenylphosphino)ferrocene-palladium dichloride (0.042 g, 0.05 mmol) was suspended in anhydrous N,N-dimethylformamide (5 mL) under an inert atmosphere. The reaction mixture was heated at about 80° C. for about 18 hours. The resulting mixture was cooled to ambient temperature and filtered through a celite pad. The crude material was concentrated in vacuo and purified by flash chromatography on silica gel using ethyl acetate:heptane (1:4) as the mobile phase. Fractions containing the desired product were combined and concentrated under the reduced pressure to afford 3-(4′-cyano)biphenylboronic acid pinacol ester as an off-white solid (0.133 g, 0.43 mmol); ¹H NMR (DMSO-d₆, 400 MHz) 1.30 (s, 12H), 7.52 (t, 1H), 7.73 (d, 1H), 7.9 (m, 6H).

### N-[4-(Biphenyl-4-ylamino)-thieno[2,3-c]pyridin-2-yl]-2,2,2-trifluoro-acetamide

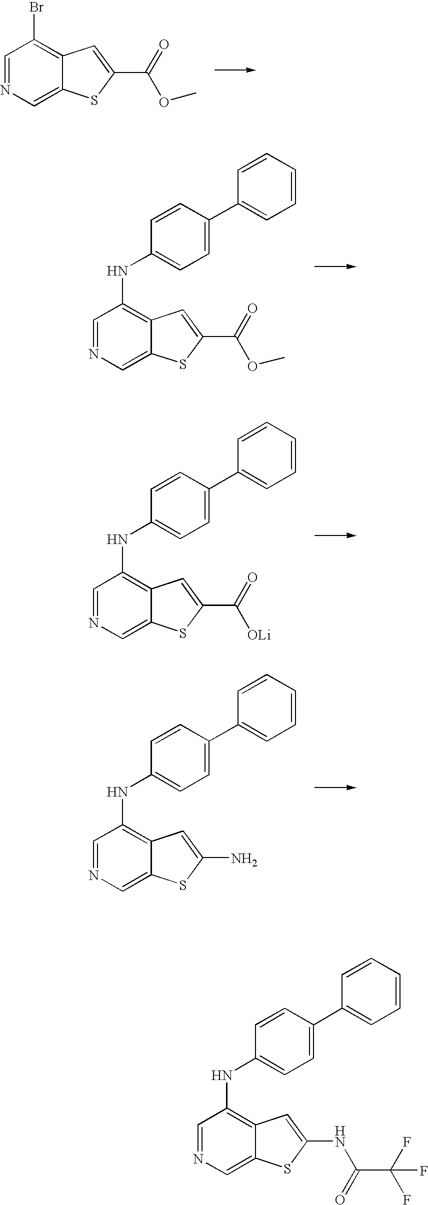

### a) 4-(Biphenyl-4-ylamino)-thieno[2,3-c]pyridine-2-carboxylic acid methyl ester

A mixture of 4-bromo-thieno[2,3-c]pyridine-2-carboxylic acid methyl ester (5.0 g, 0.0184 mol), 4-aminobiphenyl (3.41 g, 0.0202 mol), cesium carbonate (14.47 g, 0.0441 mol), 4,5-bis9diphenylphosphino0-9,9-dimethyl-xanthene (0.638 g, 0.0011 mol) and tris9dibenzylideneacetone)dipalladium (0) in 1,4-dioxane was degassed and then heated at 100° C. under continuous nitrogen flow for 16 hours. The solvent was removed under reduced pressure; the residue was triturated in water and the precipitate was collected by filtration. It was washed with ice-cold water (2×50 mL) and acetonitrile (2×40 mL) and dried to yield 4-(biphenyl-4-ylamino)-thieno[2,3-c]pyridine-2-carboxylic acid methyl ester (5.9 g, 0.0164 mol) as a yellow solid.

m/z: (M+H)⁺ 361.

### b) 4-(Biphenyl-4-ylamino)-thieno[2,3-c]pyridine-2-carboxylic acid lithium salt

To the suspension of 4-(biphenyl-4-ylamino)-thieno[2,3-c]pyridine-2-carboxylic acid methyl ester (3.84 g, 0.0107 mol) in a mixture of 1,4-dioxane (40 mL) and water (40 mL), lithium hydroxide monohydrate (0.67 g, 0.016 mol) was added and the reaction mixture was stirred at ambient temperature for 5 hours. 1,4-Dioxane was removed under reduced pressure and the precipitate was collected by filtration and dried to yield 4-(biphenyl-4-ylamino)-thieno[2,3-c]pyridine-2-carboxylic acid lithium salt (3.5 g, 0.010 mol) as a yellow solid.

m/z: (M+H)⁺ 347.

### c) N*4*-Biphenyl-4-yl-thieno[2,3-c]pyridine-2,4-diamine dihydrochloride

The mixture of 4-(biphenyl-4-ylamino)-thieno[2,3-c]pyridine-2-carboxylic acid lithium salt (3.42 g, 0.0097 mol), diphenylphosphoryl azide (2.35 mL, 0.0109 mol) and triethylamine (1.52 mL, 0.0109 mol) in t-butanol (80 mL) was heated at reflux for 8 hours while adding another 0.23 mL of diphenylphsophoryl azide and 0.5 mL of triethylamine after 5 hours. The solvent was removed under reduced pressure; the residue was suspended in ethyl acetate (100 mL) and the resulting suspension was filtered through a Celite™ pad. The solution was concentrated and dried on high vacuum pump. 0.2 g of the residue was suspended in a mixture of 1,4-dioxane (6 mL) and 3N hydrochloric acid and the mixture was heated at 80° C. for 5 hours. The reaction mixture was cooled to ambient temperature and the precipitate was collected by filtration and dried to yield N*4*-biphenyl-4-yl-thieno[2,3-c]pyridine-2,4-diamine dihydrochloride (0.112 g, 0.00029 mol) as a yellow solid.

m/z: (M+H)⁺ 318.

### d) N-[4-(Biphenyl-4-ylamino)-thieno[2,3-c]pyridin-2-yl]-2,2,2-trifluoro-acetamide

N*4*-biphenyl-4-yl-thieno[2,3-c]pyridine-2,4-diamine dihydrochloride (0.112 g, 0.00029 mol) was suspended in a mixture of anhydrous dichloromethane (5 mL) and triethylamine (0.5 mL) and trifluoroacetic anhydride (0.05 mL, 0.00035 mol) was added dropwise. The mixture was stirred at ambient temperature for 3 hours, concentrated and purified by preparative RP-HPLC (40% to 70% acetonitrile/0.05M aqueous ammonium acetate, buffered to pH 4.5, over 30 min at 21 mL/min; λ=254 nm; Microsorb C18, 100 Å, 5 μm, 250×46 mm column) to yield N-[4-(biphenyl-4-ylamino)-thieno[2,3-c]pyridin-2-yl]-2,2,2-trifluoro-acetamide (0.039 g, 0.000094 mol) as a yellow solid.

Retention time—2.56 min., RP-HPLC (30% to 95% acetonitrile/0.01M aqueous ammonium acetate, buffered to pH 4.5, over 4.5 min at 0.8 mL/min; λ=190-700 nm; Genesis CIS, 120 Å, 4 μm, 33×4.6 mm column.).

m/z: (M+H)⁺ 414.

### 3-[4-(Biphenyl-4-ylamino)-thieno[2,3-c]pyridin-2-yl]-1,1-dimethyl urea

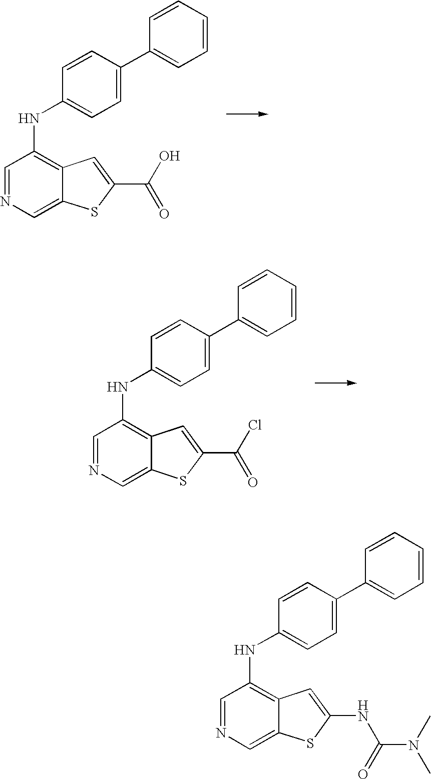

### a) 4-(Biphenyl-4-ylamino)-thieno[2,3-c]pyridine-2-carbonyl chloride

To the suspension of 4-(biphenyl-4-ylamino)-thieno[2,3-c]pyridine-2-carboxylic acid (1.0 g, 0.00289 mol) in anhydrous DCM (25 mL), oxalyl chloride (1.26 mL, 0.0145 mol) was added at 0° C. under continuous nitrogen flow followed by the addition of DMF (5 drops). The resulting suspension was stirred at ambient temperature for 18 hours and the precipitate was collected by filtration and dried to yield 4-(biphenyl-4-ylamino)-thieno[2,3-c]pyridine-2-carbonyl chloride (1.0 g, 0.00262 mol) as a yellow solid.

¹H NMR (DMSO-d₆): δ 9.85 (s, 1H), 9.2 (s, 1H), 8.91 (s, 1H), 8.23 (s, 1H), 7.76 (d, 2H), 7.71 (d, 2H), 7.5 (m, 4H), 7.34 (t, 1H).

### b) 3-[4-(Biphenyl-4-ylamino)-thieno[2,3-c]pyridin-2-yl]-1,1-dimethyl urea

The mixture of 4-(biphenyl-4-ylamino)-thieno[2,3-c]pyridine-2-carbonyl chloride (0.1 g, 0.000262 mol) and trimethylsilyl azide (0.073 mL, 0.00055 mol) was heated in carbon tetrachloride at reflux under continuous nitrogen flow for 24 hours. The solvent was removed under reduced pressure; the residue was digested with DMF (10 mL) and heated 80° C. for 44 hours. After the reaction mixture was cooled to ambient temperature, the precipitate was collected by filtration, washed with ethyl acetate (2×15 mL) and dried to yield 3-[4-(biphenyl-4-ylamino)-thieno[2,3-c]pyridin-2-yl]-1,1-dimethyl urea (0.032 g, 0.000082 mol) as a yellow solid.

Retention time—2.72 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 389.

### 1-[4-(Biphenyl-4-ylamino)-thieno[2,3-c]pyridin-2-yl]-1,4-dihydro-tetrazol-5-one

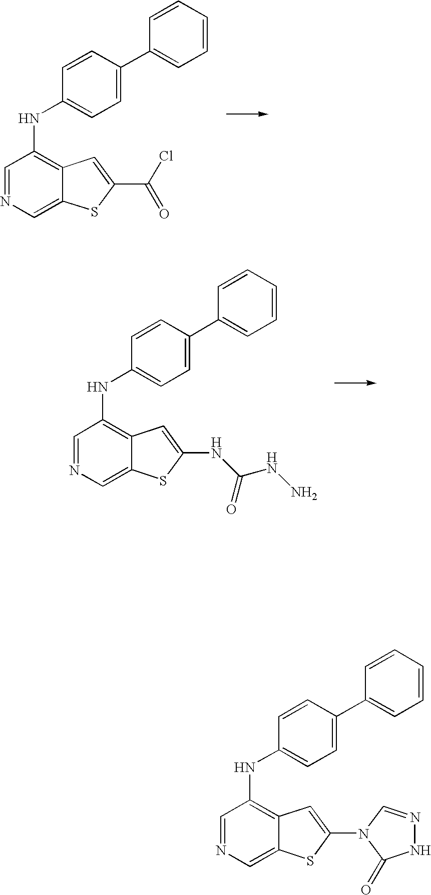

The mixture of 4-(biphenyl-4-ylamino)-thieno[2,3-c]pyridine-2-carbonyl chloride (0.1 g, 0.000262 mol) and trimethylsilyl azide (0.073 mL, 0.00055 mol) was heated in carbon tetrachloride at reflux under continuous nitrogen flow for 24 hours. The solvent was removed under reduced pressure; the residue was digested with DMF (4 mL) and sodium azide (0.05 g, 0.00077 mol) was added at once. The reaction mixture was heated at 60° C. for 2 hours, 1 mL of acetonitrile was added and the heating at 60° C. was continued for another 4 hours. The solvents were removed under reduced pressure and the residue was subjected to preparative RP-HPLC (20% to 80% 0.1% trifluoroacetic acid in acetonitrile/water, over 30 min at 21 mL/min; λ=254 nm; Microsorb C18, 100 Å, 5 μm, 250×46 mm column) to yield 1-[4-(biphenyl-4-ylamino)-thieno[2,3-c]pyridin-2-yl]-1,4-dihydro-tetrazol-5-one (0.014 g, 0.000036 mol) as a yellow solid.

Retention time—1.85 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 387.

### [4-(Biphenyl-4-ylamino)-thieno[2,3-c]pyridin-2-yl]-urea

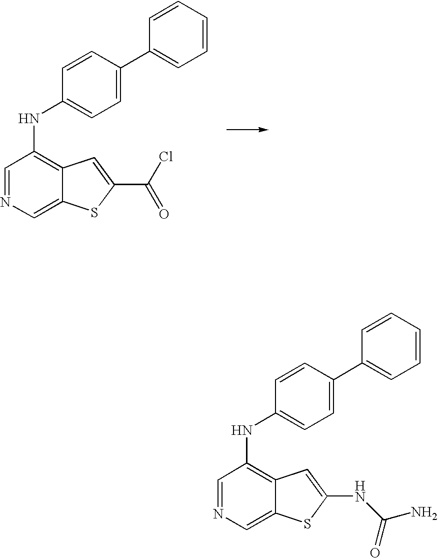

The mixture of 4-(biphenyl-4-ylamino)-thieno[2,3-c]pyridine-2-carbonyl chloride (0.1 g, 0.000262 mol) and trimethylsilyl azide (0.073 mL, 0.00055 mol) was heated in carbon tetrachloride at reflux under continuous nitrogen flow for 24 hours. The solvent was removed under reduced pressure; the residue was digested with the saturated solution of ammonia in ethanol and stirred at ambient temperature for 1 hour. The solvent was removed under reduced pressure and the residue was subjected to preparative RP-HPLC (20% to 50% acetonitrile/0.05M aqueous ammonium acetate, buffered to pH 4.5, over 30 min at 21 mL/min; λ=254 nm; Microsorb C18, 100 Å, 5 μm, 250×46 mm column) to yield [4-(biphenyl-4-ylamino)-thieno[2,3-c]pyridin-2-yl]-urea (0.025 mg, 0.0000694 mol) as a yellow solid.

Retention time—2.29 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 361.

### 4-[4-(Biphenyl-4-ylamino)-thieno[2,3-c]pyridin-2-yl]-2,4-dihydro-[1,2,4]triazol-3-one

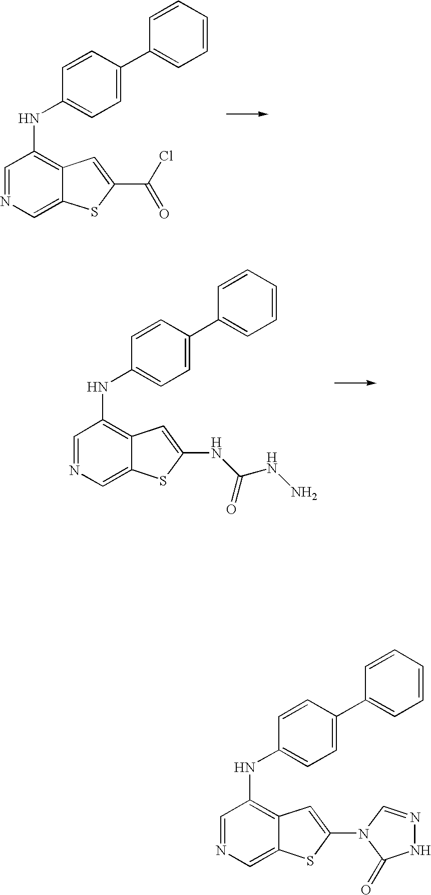

### a) N-[4-(biphenyl-3-ylamino)thieno[2,3-c]pyridine-2-yl]hydrazinecarboxamide

The mixture of 4-(biphenyl-4-ylamino)-thieno[2,3-c]pyridine-2-carbonyl chloride (0.1 g, 0.000262 mol) and trimethylsilyl azide (0.073 mL, 0.00055 mol) was heated in carbon tetrachloride at reflux under continuous nitrogen flow for 24 hours. The solvent was removed under reduced pressure; the residue was digested with ethanol(20 mL) and hydrazine (0.3 mL) was added dropwise. The reaction mixture was stirred at ambient temperature for 1 hour, filtered through a Celite™ pad and concentrated to yield N-[4-(biphenyl-3-ylamino)thieno[2,3-c]pyridine-2-yl]hydrazinecarboxamide (0.087 g, 0.000232 mol) as a yellow solid.

m/z: (M+H)⁺ 376.

### b) 4-[4-(Biphenyl-4-ylamino)-thieno[2,3-c]pyridin-2-yl]-2,4-dihydro-[1,2,4]triazol-3-one

c) The mixture of N-[4-(biphenyl-3-ylamino)thieno[2,3-c]pyridine-2-yl]hydrazinecarboxamide (0.15 g, 0.0004 mol) and formamidine acetate (0.208 g, 0.002 mol) in DMF (5 mL) was stirred at ambient temperature under continuous nitrogen flow for 1 hour. Acetic acid (0.11 mL, 0.002 mol) was added and the resulting mixture was heated at 80° C. for 16 hours. The solvent was removed and the residue subjected to preparative RP-HPLC (20% to 50% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 4-[4-(Biphenyl-4-ylamino)-thieno[2,3-c]pyridin-2-yl]-2,4-dihydro-[1,2,4]triazol-3-(0.018 g, 0.000047 mol) as a yellow solid.

Retention time—2.48 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M+H)⁺ 386.

### 5-Biphenyl-4-ylmethyl-imidazo[1,2-a]pyrazine-2-carboxylic acid

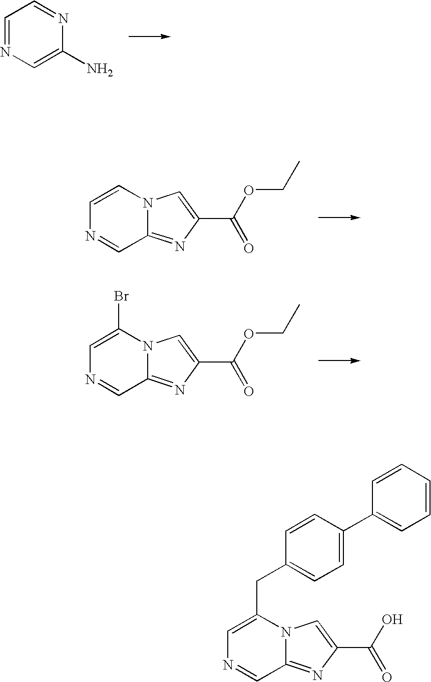

### a) Imidazo[1,2-a]pyrazine-2-carboxylic acid ethyl ester

The mixture of aminopyrazine (3.0 g, 0.03316 mol) and 2-bromoethylpyruvate (4.77 mL, 0.0379 mol) in anhydrous ethanol (150 mL) was heated under continuous nitrogen flow at reflux for 6 hours. The reaction mixture was treated with charcoal (10 g), filtered through a Celite™ pad and concentrated under reduced pressure down to 25 mL. The solution was added dropwise to the saturated solution of sodium bicarbonate in water (300 mL) and the aqueous layer was extracted with DCM (3×200 mL). The combined organic extracts were dried with magnesium sulfate and concentrated. The residue was suspended in ether (40 mL) and the precipitate was collected by filtration and dried to yield imidazo[1,2-a]pyrazine-2-carboxylic acid ethyl ester (1.4 g, 0.0073 mol) as an off-white solid.

m/z: (M+H)⁺ 192.

### b) 5-Bromo-imidazo[1,2-a]pyrazine-2-carboxylic acid ethyl ester

To the suspension of imidazo[1,2-a]pyrazine-2-carboxylic acid ethyl ester (0.3 g, 0.0016 mol) in anhydrous ethanol (7 mL), the chilled solution of bromine (0.16 mL, 0.0032 mol) in anhydrous ethanol (7 mL) was added dropwise and the resulting mixture was heated at reflux under continuous nitrogen flow for 1.5 hours. The reaction mixture was concentrated under reduced pressure and treated with the saturated solution of sodium bicarbonate in water (50 mL). The aqueous layer was extracted with ethyl acetate (3×25 mL), the combined organic extracts were dried with magnesium sulfate and concentrated. The residue was suspended in heptane (30 mL) and the precipitate was collected by filtration and dried to yield 5-bromo-imidazo[1,2-a]pyrazine-2-carboxylic acid ethyl ester (0.075 g, 0.00028 mol) as a yellow solid.

m/z: (M+H)⁺ 270,272.

### c) 5-Biphenyl-4-ylmethyl-imidazo[1,2-a]pyrazine-2-carboxylic acid

The mixture of 5-bromo-imidazo[1,2-a]pyrazine-2-carboxylic acid ethyl ester (0.3 g, 0.0011 mol), 3-biphenyl boronic acid (0.329 g, 0.0017 mol), (2′,6′-dimethoxy-biphenyl-2-yl)-dicyclopentadienyl-phosphane (0.091 g, 0.00022 mol), palladium acetate (0.025g, 0.00011 mol) and potassium phosphate (0.71 g, 0.0033 mol) in terahydrofuran (7 mL) was stirred at ambient temperature under continuous nitrogen flow for 20 hours. A solution of lithium hydroxide monohydrate (0.2g, 0.0048 mol) in 5 mL of water was added and the stirring at ambient temperature was continued for another 4 hours. The solvents were removed and the residue was subjected to to preparative RP-HPLC (10% to 40% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 5-biphenyl-4-ylmethyl-imidazo[1,2-a]pyrazine-2-carboxylic acid (0.049 g, 0.00016 mol) as an off-white solid.

Retention time—1.22 min., RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 4 μm, 33×4.6 mm column.).

m/z: (M−H)⁻ 314.

### Preparation 154: 3-[4-(Biphenyl-4-ylamino)-thieno[2,3-c]pyridin-2-yl]-4H-[1,2,4]oxadiazin-5-one

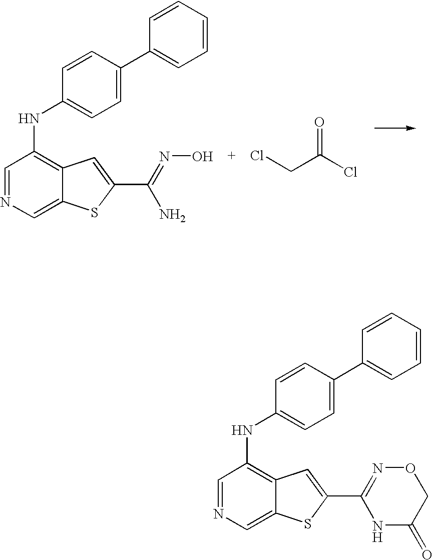

To a suspension of 4-(Biphenyl-4-ylamino)-N-hydroxy-thieno[2,3-c]pyridine-2-carboxamidine (0.180 g, 0.500 mmol) and sodium carbonate (0.196 g, 1.85 mmol) in dichloromethane (8 mL) was added chloroacetyl chloride (95 μL, 1.25 mmol) dropwise. A color change from bright yellow to deep orange was noted as the reaction was stirred at room temperature for 15 minutes. The solvent was removed under reduced pressure and the crude precipitate was then dissolved in tetrahydrofuran (8 mL). Sodium hydride, 60% dispersion in mineral oil (60 mg, 1.5 mmol) was added in small portions. The reaction mixture was stirred at room temperature for 30 minutes and the solvent was removed under reduced pressure. The crude solid was triturated with ether (15 mL×3), ethyl acetate (15 mL×3), and acetonitrile (15 mL×3), respectively, to give 49.1 mg of the desired product, 3-[4-(Biphenyl-4-ylamino)-thieno[2,3-c]pyridin-2-yl]-4H-[1,2,4]oxadiazin-5-one, as deep yellow solid. RP-HPLC (table 1, method J) Rₜ 6.26 min; m/z: (M+H)⁺ 401.

### Preparation 155: 2-[4-(Biphenyl-4-ylamino)-thieno[2,3-c]pyridin-2-yl]-4H-[1,3,4]oxadiazin-5-one

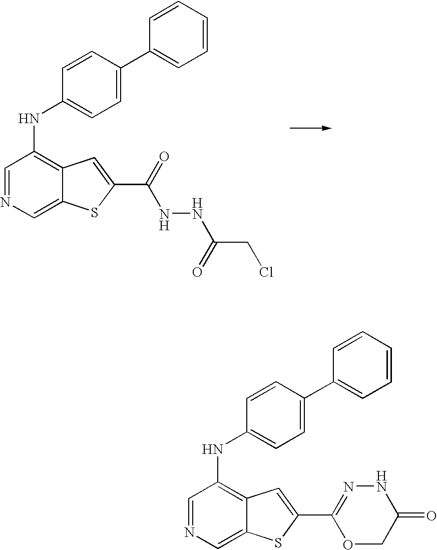

To a solution of 4-(Biphenyl-4-ylamino)-thieno[2,3-c]pyridine-2-carboxylic acid N′-(2-chloro-acetyl)-hydrazide (0.218 g, 0.500 mmol) in anhydrous dimethylformamide (12 mL) was added potassium iodide (91 mg, 0.55 mmol). The reaction was stirred at 60° C. for 15 minutes, at which time sodium bicarbonate (0.168 g, 2.00 mmol) was added to the mixture. The reaction mixture was stirred for another 5 hours and the solvent was removed under reduced pressure to obtain a brown solid. The crude product was purified by preparative RP-HPLC (30% to 80% acetonitrile/0.05M aqueous ammonium acetate, buffered to pH 4.5, over 30 min at 21 mL/min; λ=254 nm; Hypersil C18, 100 Å, 8 μm, 250×21.1 mm column) to give 26 mg of the desired product, 2-[4-(Biphenyl-4-ylamino)-thieno[2,3-c]pyridin-2-yl]-4H-[1,3,4]oxadiazin-5-one. RP-HPLC (table 1, method J) Rₜ 6.29 min; m/z: (M+H)⁺ 401.

### Preparation 156: 5-[4-(Biphenyl-4-ylamino)-thieno[2,3-c]pyridin-2-yl]-4-ethyl-2,4-dihydro-[1,2,4]triazol-3-one

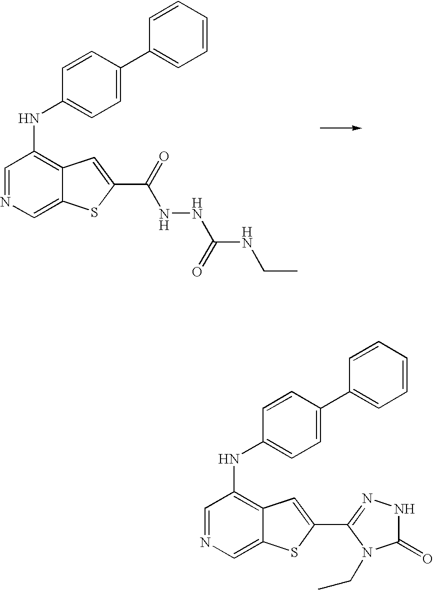

A mixture of (ethylamino)-N-({4-[(4-phenylphenyl)amino]thiopheno[2,3-c]pyridin-2-yl}carbonylamino)carboxamide (0.215 g, 0.500 mmol) and potassium carbonate (0.207 g, 1.50 mmol) in distilled water (12 mL) was stirred at 90° C. for 4 days and at room temperature for 3 days. Water was removed under reduced pressure and the crude solid was dissolved in dimethylformamide (10 mL) and filtered through a pad of celite. The crude product was purified by preparative RP-HPLC (30% to 70% acetonitrile/0.05M aqueous ammonium acetate, buffered to pH 4.5, over 30 min at 21 mL/min; λ=254 nm; Hypersil C18, 100 Å, 8 μm, 250×21.1 mm column) to give 18 mg of the desired product, 5-[4-(Biphenyl-4-ylamino)-thieno[2,3-c]pyridin-2-yl]-4-ethyl-2,4-dihydro-[1,2,4]triazol-3-one as yellow needles. RP-HPLC (table 1, method ?) Rₜ 5.99 min; m/z: (M+H)⁺ 414.

### Preparation #157: 4-(Biphen-3-yl)-5-chloro-thieno[2,3-c]pyridine-2-carboxylic acid methyl ester

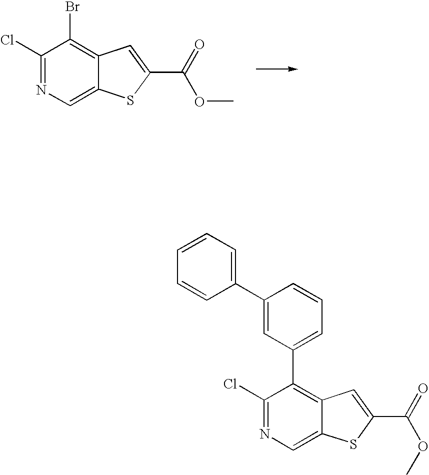

Following general procedure II, 4-Bromo-5-chloro-thieno[2,3-c]pyridine-2-carboxylic acid methyl ester (0.025 g, 0.08 mmol), 3-Biphenylboronic acid (0.016 g, 0.08 mmol), PdCl₂ dppf (0.035 g, 0.0.003 mmol) and cesium carbonate (0.03 g, 0.08 mmol) were combined in DME/water (5:1, 1 mL). The reaction mixture was purged with nitrogen and heated at about 100° C. in a sealed vessel for about 5 hours. The reaction mixture was cooled to ambient temperature, diluted into 2 mL DMSO then filtered. The solution was purified by reverse phase HPLC to yield 4-(biphen-3-yl)-5-chloro-thieno[2,3-c]pyridine-2-carboxylic acid methyl ester as a tan solid (0.02 g, 0.05 mmol); RP-HPLC (Table 1, Method i) Rₜ=4.04 min; m/z: (M+H)⁺ 380.

### Preparation #158: 5-Amino-4-(biphen-3-yl)-thieno[2,3-c]pyridine-2-carboxylic acid,

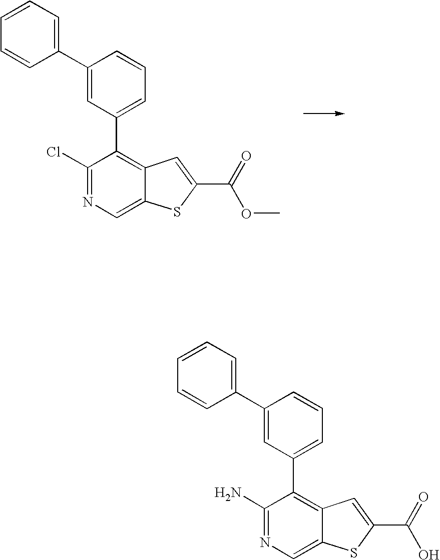

Following general procedure I, 4-(Biphen-3-yl)-5-chloro-thieno[2,3-c]pyridine-2-carboxylic acid methyl ester (0.02 g, 0.05 mmol), benzophenone imine (0.01 g, 0.06 mmol), 9,9-dimethyl-4,5-bis(diphenylphosphino)xanthene (0.002 g, 0.005 mmol), Pd₂dba₃ (0.0018 g, 0.0025 mmol) and cesium carbonate (0.032 g, 0.10 mmol) were combined in 1,4-dioxane (1 mL). The mixture was purged with nitrogen and heated in a sealed tube at about 100° C. for about 16 hours. The reaction was cooled to r.t., treated with 3M HCl (5.0 mL) and stirred at about 60° C. for about 1 hour. The reaction mixture was diluted into 3 mL DMSO and filtered. The crude product was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 5-amino-4-(biphen-3-yl)-thieno[2,3-c]pyridine-2-carboxylic acid (0.003 g, 0.009 mmol) as a tan solid; RP-HPLC (Table 1, Method i) Rₜ=1.26 min; m/z: (M+H)⁺ 347, (M−H)⁻ 345 . Also isolated starting material that was hydrolyzed to the correspondine carboxylic acid. 5-chloro-4-(biphen-3-yl)-thieno[2,3-c]pyridine-2-carboxylic acid (0.007 g, 0.019 mmol) as a tan solid; RP-HPLC (Table 1, Method i) Rₜ=1.70 min; m/z: (M−H)⁻ 364.

### Preparation #159: 5-Chloro-thieno[2,3-c]pyridine-2-carboxylic acid methyl ester

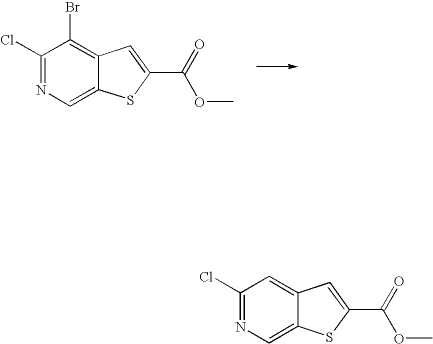

4-Bromo-5-chloro-thieno[2,3-c]pyridine-2-carboxylic acid methyl ester (0.05 g, 0.16 mmol) is suspended in about 5 mL EtOH and 10% Pd on carbon (0.01 g) is added. The reaction mixture was purged with nitrogen and exposed to hydrogen gas in a sealed vessel at about 10 psi for about 48 hours. The reaction mixture was filtered and the solution was purified by reverse phase HPLC to yield 5-Chloro-thieno[2,3-c]pyridine-2-carboxylic acid methyl ester as a pale yellow solid (0.005 g, 0.02 mmol); RP-HPLC (Table 1, Method i) Rₜ=1.86 min; ¹H NMR (DMSO-d₆): δ 9.25 (s, 1H), 8.21 (s, 1H), 8.15 (s, 1H), 3.92 (s, 3H).

### Preparation #160: 5-Amino-4-(biphen-3-yl)-thieno[2,3-c]pyridine-2-carboxylic acid,

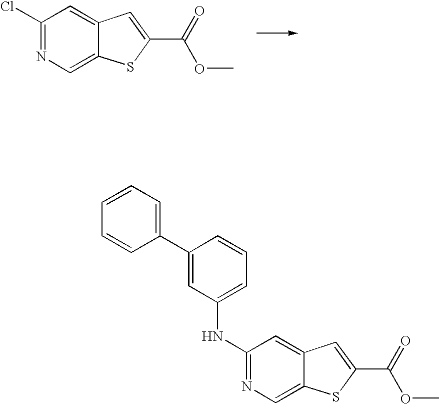

Following general procedure I, 5-chloro-thieno[2,3-c]pyridine-2-carboxylic acid methyl ester (0.005 g, 0.02 mmol), biphenyl-3-yl-amine (0.00037 g, 0.02 mmol), 9,9-dimethyl-4,5-bis(diphenylphosphino)xanthene (0.001 g, 0.002 mmol), Pd₂dba₃ (0.0005 g, 0.0006 mmol) and cesium carbonate (0.017 g, 0.05 mmol) were combined in 1,4-dioxane (1 mL). The mixture was purged with nitrogen and heated in a sealed tube at about 100° C. for about 24 hours. The reaction was cooled to r.t., diluted into 3 mL DMSO and filtered. The crude product was purified by preparative RP-HPLC (20%-100% acetonitrile/0.05M aqueous ammonium acetate, buffered to pH 4.5, over 25 min at 15 mL/min; λ=254 nm; Hypersil C18, 100 Å, 8 μm, 250×21.2 mm column) to yield 5-(Biphenyl-3-ylamino)-thieno[2,3-c]pyridine-2-carboxylic acid methyl ester (0.002 g, 0.006 mmol) as a tan solid; RP-HPLC (Table 1, Method i) Rₜ=2.48 min; m/z: (M+H)⁺ 361, (M−H)⁻ 359.

### Preparation #161: N-(3-boronic acid-2-yl-phenyl)-benzamide

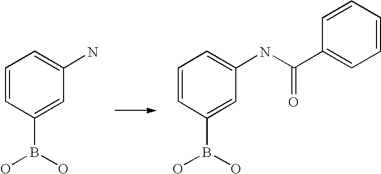

3-aminophenyl boronic acid (0.5 g, 2.9 mmol) is suspended in about 5 mL DCM and benzoyl chloride (0.51 g, 3.6 mmol) is added. The reaction mixture was cooled to about 0° C. for about 10 minutes then DIPEA (1.5 mL, 8.7 mmol) was introduced dropwise. The reaction was stirred at ambient temperature for about 18 hours. The reaction mixture was purified by silica gel chromatography to yield N-(3-boronic acid-2-yl-phenyl)-benzamide as an oil. Upon standing the product crystallized to a white solid (0.22 g, 0.9 mmol); RP-HPLC (Table 1, Method i) Rₜ=3.95 min, 242 (M+H)⁺, 240 (M−H).

### Preparation #162: 1-Phenyl-3-[4-(4,4,5,5-tetramethyl-[1,3,2]dioxaborolan-2-yl)-phenyl]-urea

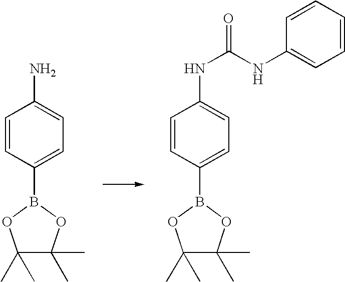

4-(4,4,5,5-Tetramethyl-[1,3,2]dioxaborolan-2-yl)-phenylamine (1.0 g, 4.6 mmol) is suspended in about 5 mL DCM and phenyl isocyanate (0.5 mL, 4.6 mmol) is added dropwise at ambient temperature. The reaction mixture was stirred at ambient temperature for about 18 hours. The reaction mixture was filtered to remove product. A white solid was isolated and identified as 1-Phenyl-3-[4-(4,4,5,5-tetramethyl-[1,3,2]dioxaborolan-2-yl)-phenyl]-urea (1.0 g, 2.9 mmol); RP-HPLC (Table 1, Method i) Rₜ=2.36 min, 339 (M+H)⁺, 337 (M−H).

### Preparation #163: 2-Phenyl-N-[4-(4,4,5,5-tetramethyl-[1,3,2]dioxaborolan-2-yl)-phenyl]-acetamide

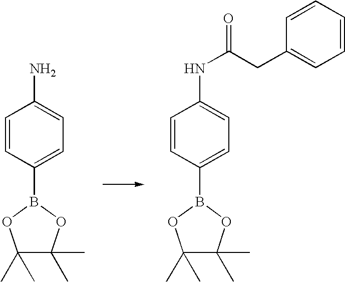

4-(4,4,5,5-Tetramethyl-[1,3,2]dioxaborolan-2-yl)-phenylamine (1.0 g, 4.6 mmol) is suspended in about 50 mL DCM and pyridine (0.35 mL, 4.6 mmol) is added. The solution is stirred at ambient temperature under nitrogen. Next phenylacetyl chloride (0.6 mL, 4.6 mmol) is added dropwise at ambient temperature. The reaction mixture was stirred at ambient temperature for about 18 hours. The reaction mixture was concentrated to a light yellow oil. Upon standing the product crystallized giving a light yellow waxy solid. Following trituration with heptanes, then water and drying, a pale yellow solid was isolated. 2-Phenyl-N-[4-(4,4,5,5-tetramethyl-[1,3,2]dioxaborolan-2-yl)-phenyl]-acetamide (1.2 g, 3.6 mmol); RP-HPLC (Table 1, Method i) Rₜ=2.35 min, 338 (M+H)⁺, 336 (M−H).

### Preparation #164: Preparation of 4-Bromo-3-methyl-thieno[2,3-c]pyridine-2-carboxylic acid methyl ester

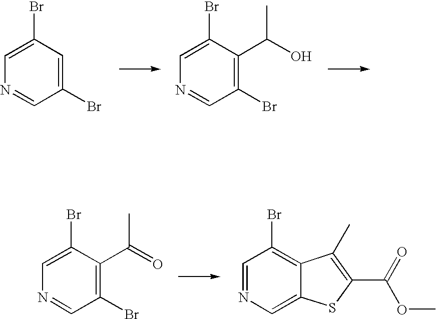

To a solution of di-isopropylamine (5.2 g, 52 mmol) in THF was added, at −78° C., n-BuLi in heptanes (2.5M, 48 mmol) and held at −78° C. for about 30 minutes. A solution of 3,5-dibromopyridine (8.7 g. 37 mmol) in THF was added and the resulting solution was stirred an additional 30 minutes. Acetaldehyde (8 g, 18 mmol) was introduced in THF and the mixture was stirred to ambient temperature then held for about 12 hours. The resulting crude reaction mixture was quenched with NaHCO₃ then separated in water and EtOAc. Following further extraction with EtOAc, the combined organics were washed with saturated NaCl then dried over Na₂SO₄. The crude product was further purified via chromatography on silica gel (7:3 heptane/EtOAc as eluent) to afford 1-(3,5-Dibromo-pyridin-4-yl)-ethanol which was carried on to the next step.

To a solution of 1-(3,5-Dibromo-pyridin-4-yl)-ethanol (1 g, 3.6 mmol) in 12 mL dichloromethane was added Dess-Martin periodinane (1.8 g, 4.4 mmol) at 0° C. The solution was warmed to RT over about 30 minutes, then diluted in dichloromethane (20 mL) and passed over a column of silica gel. Concentration gives 1-(3,5-Dibromo-pyridin-4-yl)-ethanone (1 g, 3.6 mmol), which is carried on to the next step.

To 1-(3,5-Dibromo-pyridin-4-yl)-ethanone (3 g, 11 mmol) in THF (60 mL) was added cesium carbonate (10.6 g, 33 mmol) and methylthioglycolate (1.7 g, 16 mmol). The resulting mixture was heated at about 60° C. for about 2 hours. The reaction mixture was cooled to ambient temperature and partially concentrated in vacuo. The crude was washed diluted into EtOAc, washed with dilute NaHCO₃ (aq) then purified via chromatography on silica (7:3 heptane/EtOAc as eluent) to afford 4-Bromo-3-methyl-thieno[2,3-c]pyridine-2-carboxylic acid methyl ester (1.86 g, 6.5 mmol) as a off-white solid; RP-HPLC (Table 1, Method i), Rₜ 7.13 min; m/z: (M+H)⁺ 286, 288.

### Preparation #165: Preparation of 4-(4-Bromo-phenylamino)-3-methyl-thieno[2,3-c]pyridine-2-carboxylic acid methyl ester

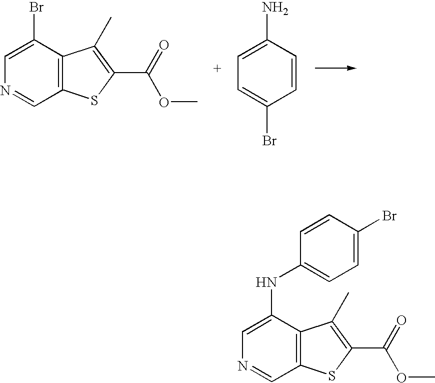

To a solution of 4-Bromo-3-methyl-thieno[2,3-c]pyridine-2-carboxylic acid methyl ester (prepared using preparation CB1) (0.25 g, 0.8 mmol) in anhydrous dioxane (4.5 mL) was added 4-bromoaniline (0.165 g, 0.096 mmol), cesium carbonate (0.852 g, 2.6 mmol), and XANTPHOS (0.05 g, 0.087 mmol). The mixture was stirred and nitrogen gas was bubbled through the suspension for about five minutes at ambient temperature.

Tris(dibenzylideneacetone)dipalladium (0) (0.08 g, 0.079 mmol) was added. Nitrogen gas was then bubbled through the resulting mixture for five minutes and the reaction was heated at about 110° C. for about 18 hours. The reaction mixture was cooled to ambient temperature, diluted with DMF (3 mL) and filtered through a Celite® pad. The crude filtrate was purified via preparative RP-HPLC (Table 1, Method k) to afford 4-(4-Bromo-phenylamino)-3-methyl-thieno[2,3-c]pyridine-2-carboxylic acid methyl ester (0.15 g, 0.40 mmol) as a yellow solid; RP-HPLC Rₜ 3.46 min (Table 1, Method i); m/z: (M+H)⁺ 377,379, (M−H)⁻ 375,377.

### Preparation #166: Preparation of 4-(4-Bromo-phenylamino)-3-methyl-thieno[2,3-c]pyridine-2-carboxylic acid amide

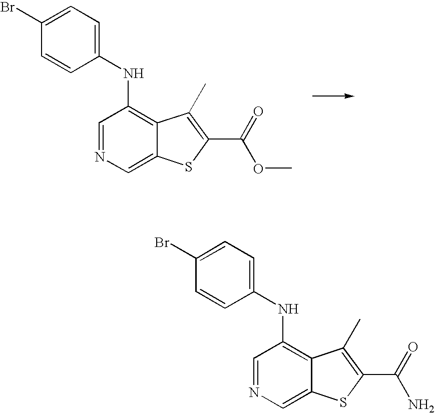

A solution of 4-(4-Bromo-phenylamino)-3-methyl-thieno[2,3-c]pyridine-2-carboxylic acid methyl ester (0.150 g, 0.4 mmol) in 10 mL of 7 N ammonia/MeOH was heated at about 80° C. for about 4 hours. The reaction mixture was cooled to r.t., concentrated and filtered to provide 4-(4-Bromo-phenylamino)-3-methyl-thieno[2,3-c]pyridine-2-carboxylic acid amide (0.035 g, 0.10 mmol) as a yellow solid; RP-HPLC Rₜ 6.34 (Table 1, Method i); m/z: (M+H)⁺ 362, 364, (M−H)⁻ 360, 361.

### Preparation #167: Preparation of 4-(4-Bromo-phenylamino)-3-methyl-thieno[2,3-c]pyridine-2-carboxylic acid

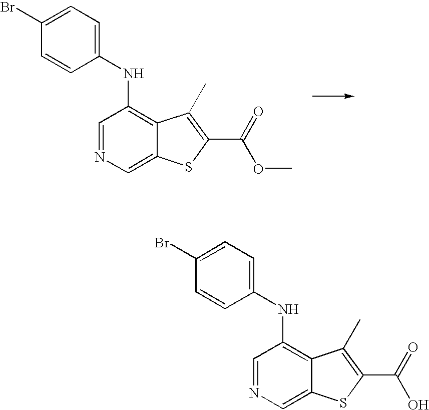

To a solution of 4-(4-Bromo-phenylamino)-3-methyl-thieno[2,3-c]pyridine-2-carboxylic acid methyl ester (0.020 g, 0.053 mmol) in THF was added 30 % aq NaOH. The solution was stirred at ambient temperature for about 4 hours. The reaction mixture concentrated, diluted into 3 mL DMF and purified by preparative RP-HPLC (Table 1, method k) to afford 4-(4-Bromo-phenylamino)-3-methyl-thieno[2,3-c]pyridine-2-carboxylic acid (0.002 g, 0.005 mmol) as a yellow solid; RP-HPLC Rₜ 4.84 min (Table 1, Method i); m/z: (M+H)⁺ 363, 365, (M−H)⁻ 361, 363.

### Preparation #168: Preparation of 4-Biphenyl-3-yl-1H-pyrazolo[3,4-c]pyridin-3-ylamine

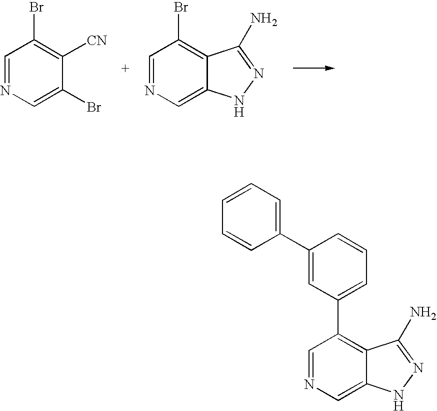

To a solution of 3,5-Dibromo-isonicotinonitrile (0.500 g, 1.9 mmol) in MeOH was added hydrazine hydrate (0.09 mL, 2 mmol). The solution was heated at about 100° C. for about 24 hours. The mixture was allowed to cool to ambient temperature and the solvents were removed under reduced pressure. The residue was further purified using preparative RP-HPLC (Table 1, Method k) to afford 4-Bromo-1H-pyrazolo[3,4-c]pyridin-3-ylamine (0.26 g, 0.08 mmol) which was used in the following step. To solution of 4-Bromo-1H-pyrazolo[3,4-c]pyridin-3-ylamine (0.09 g, 0.4 mmol) in 2.5 mL DME/water (10:1) was added PdCl₂-dppf (0.003 g, 0.01 mmol), Cs₂CO₃ (0.16 g, 0.5 mmol), and 2-biphenyl-3-yl-boronic acid (0.066 g, 0.34 mmol). The mixture was purged with nitrogen gas then heated at about 150° C. for about 20 minutes in the microwave (250 watts maximum power). The mixture was allowed to cool to ambient temperature, diluted with DMF and filtered through Celite. The residue was further purified using preparative RP-HPLC (Table 1, Method k) to afford 4-Biphenyl-3-yl-1H-pyrazolo[3,4-c]pyridin-3-ylamine (0.008 g, 0.08 mmol); RP-HPLC Rₜ 2.28 min (Table 1, Method i); m/z: (M+H)⁺ 287, (M−H)⁻ 285.

### Preparation #169: Preparation of 4-Biphenylen-1-yl-2-(1H-tetrazol-5-yl)-thieno[2,3-c]pyridine

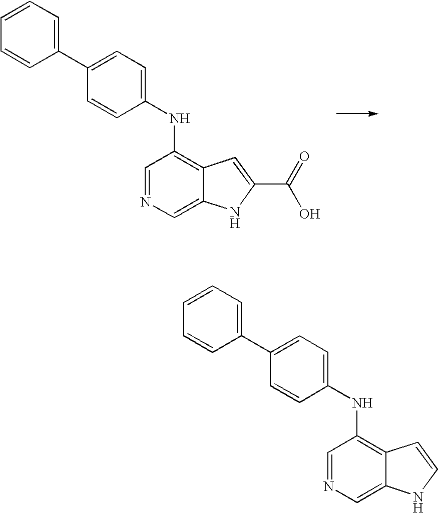

To a solution of 4-(Biphenyl-4-ylamino)-1H-pyrrolo[2,3-c]pyridine-2-carboxylic acid (0.153 g, 0.46 mmol) in degassed quinoline (5.0 mL) was added copper metal (0.015 g, 0.23 mmol) at room temperature under an atmosphere of nitrogen. The reaction mixture was heated at about 230° C. for about 4 hours. The mixture was allowed to cool to ambient temperature. The residue was purified by preparative RP-HPLC (Table 1, method k) to give biphenyl-4-yl-(1H-pyrrolo[2,3-c]pyridin-4-yl)-amine as a white powder (0.026 g, 0.091 mmol); RP-HPLC (Table 1, Method i) Rₜ 2.65 min; m/z: (M+H)⁺ 286, (M−H)⁻ 284.

### Preparation #170: Preparation of 4-Biphenyl-3-yl-1-(2-ethoxycarbonyl-methyl)-1H-pyrrolo[2,3-c]pyridine-2-carboxylic acid methyl ester

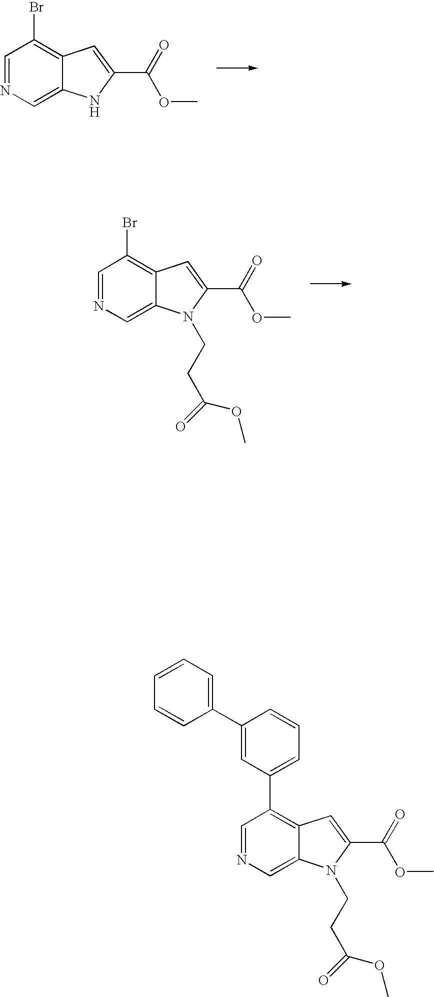

To a solution of 4-Bromo-1H-pyrrolo[2,3-c]pyridine-2-carboxylic acid methyl ester (0.500 g, 1.96 mmol) in DMF was added K₂CO₃ (0.8 g, 5.9 mmol), tetrabutylammonium iodide (0.36 g, 1 mmol), and 3-Bromo-propionic acid ethyl ester (0.425 g, 2.5 mmol),. The solution was heated at about 60-80° C. for about 24 hours. The mixture was allowed to cool to ambient temperature and the solution was further purified using preparative RP-HPLC (Table 1, Method k) to afford 4-Bromo-1-(2-ethoxycarbonyl-methyl)-1H-pyrrolo[2,3-c]pyridine-2-carboxylic acid methyl ester (0.34 g, 0.98 mmol) as a yellow solid; RP-HPLC Rₜ 2.72 min (Table 1, Method i); m/z: (M+H)⁺ 341; which was used in the following step. To solution of 4-Bromo-1-(2-ethoxycarbonyl-methyl)-1H-pyrrolo[2,3-c]pyridine-2-carboxylic acid methyl ester (0.33 g, 0.97 mmol) in 10 mL DME/water (10:1) was added Pd(PPh₃)₄ (0.1 g, 0.1 mmol), Cs₂CO₃ (0.95 g, 2.9 mmol), and 2-biphenyl-3-yl-boronic acid (0.25 g, 1.3 mmol). The mixture was purged with nitrogen gas then heated at about 100° C. for about 12-24 hours. The mixture was allowed to cool to ambient temperature, diluted with DMF and filtered through Celite. The residue was further purified using preparative RP-HPLC (Table 1, Method k) to afford 4-Biphenyl-3-yl-1-(2-ethoxycarbonyl-methyl)-]H-pyrrolo[2,3-c]pyridine-2-carboxylic acid methyl ester (0.337 g, 0.81 mmol); RP-HPLC Rₜ 5.01 min (Table 1, Method n).

### Preparation #171: Preparation of 4-Biphenyl-3-yl-1-(2-carboxy-ethyl)-1H-pyrrolo[2,3-c]pyridine-2-carboxylic acid

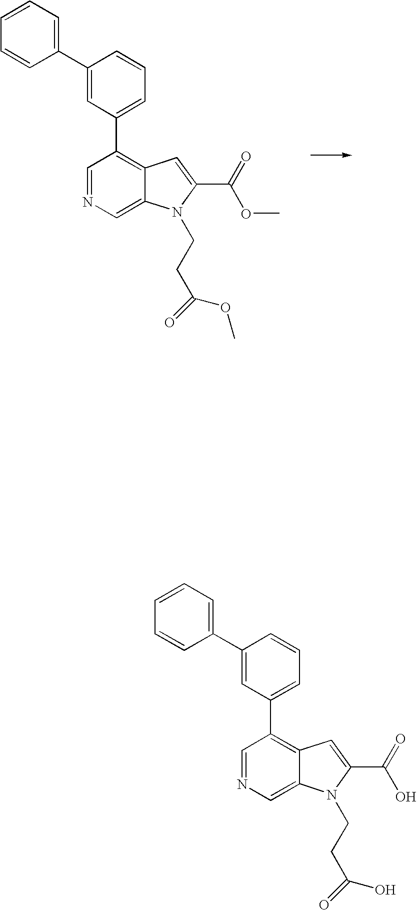

To a solution of 4-Biphenyl-3-yl-1-(2-ethoxycarbonyl-methyl)-1H-pyrrolo[2,3-c]pyridine-2-carboxylic acid methyl ester (0.24 g, 0.57 mmol) in THF/water (2 mL, 10:1) was added LiOH monohydrate (0.035 g, 0.86 mmol). The solution was stirred at ambient temperature for about 10 minutes. The reaction mixture is concentrated, diluted into 3 mL DMF and purified by preparative RP-HPLC (Table 1, method k) to afford 4-Biphenyl-3-yl-1-(2-carboxy-ethyl)-1H-pyrrolo[2,3-c]pyridine-2-carboxylic acid (0.048 g, 0.12 mmol) as a yellow solid; RP-HPLC Rₜ 0.75 min (Table 1, Method i); m/z: (M+H)⁺ 387.

### Preparation #172: Preparation of 4-Biphenyl-3-yl-1-(2-carbamoyl-ethyl)-1H-pyrrolo[2,3-c]pyridine-2-carboxylic acid amide

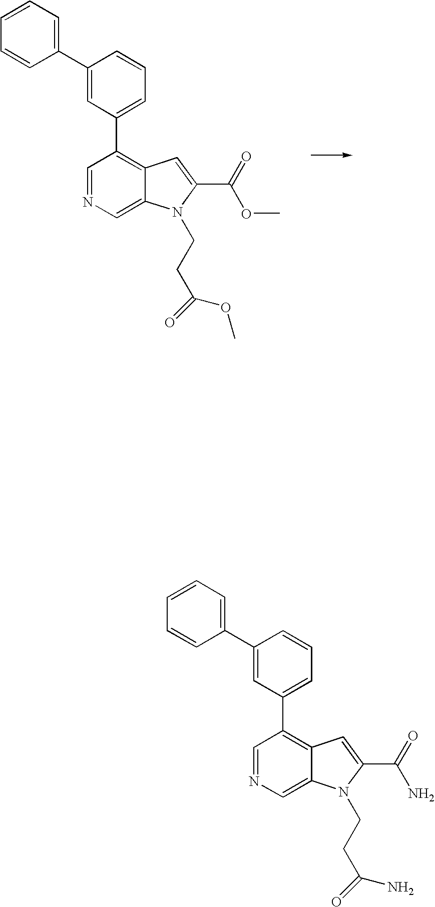

A solution of 4-Biphenyl-3-yl-1-(2-ethoxycarbonyl-methyl)-1 H-pyrrolo[2,3-c]pyridine-2-carboxylic acid methyl ester (0.10 g, 0.24 mmol) in 10 mL of 7 N ammonia/MeOH was heated at about 60° C. for about 4 hours. The reaction mixture was cooled to r.t., concentrated and purified by preparative RP-HPLC (Table 1, method k) to afford 4-Biphenyl-3-yl-1-(2-carbamoyl-ethyl)-1H-pyrrolo[2,3-c]pyridine-2-carboxylic acid amide (0.035 g, 0.10 mmol) as a yellow solid; RP-HPLC Rₜ 3.84 (Table 1, Method n).

We claim:

1. A compound or pharmaceutically acceptable salts thereof having an IC₅₀ of about 20 μM or less in a COT phosphorylation assay in macrophages.

2. A compound or pharmaceutically acceptable salts thereof according to claim 1 wherein said compound also has at least one of the following properties:
a) inhibits pErk signaling resulting from LPS stimulation in a macrophage with an EC₅₀ of about 6 μM or less;
b) inhibits TNF-alpha production resulting from LPS stimulation in macrophages with an EC₅₀ of about 20 μM or less;
c) inhibits IL-1 production resulting from LPS stimulation in macrophages with an EC₅₀ of about 20 μM or less;
d) inhibits TNF-alpha production resulting from LPS stimulation in macrophages in the presence of plasma with an EC₅₀ of about 100 μM or less;
e) inhibits IL-1 production resulting from LPS stimulation in macrophages in the presence of plasma with an EC₅₀ of about 100 μM or less;
f) inhibits LPS induced TNF-alpha in a mouse with an ED₅₀ of about 100 mg/kg or less;
g) inhibits LPS induced IL-1 in a mouse with an ED50 of about 100 mg/kg or less; or
h) inhibits collagen induced arthritis in a mouse with an ED₅₀ of about 500 mg/kg/day or less.

3. A compound or pharmaceutically acceptable salts thereof according to claim 1 wherein said compound also inhibits pErk signaling resulting from LPS stimulation in a macrophage with an EC₅₀ of about 6 μM or less.

4. A compound or pharmaceutically acceptable salts thereof according to claim 1 wherein said compound also inhibits TNF-alpha production resulting from LPS stimulation in macrophages with an EC₅₀ of about 20 μM or less.

5. A compound or pharmaceutically acceptable salts thereof according to claim 1 wherein said compound also inhibits IL-1 production resulting from LPS stimulation in macrophages with an EC₅₀ of about 20 μM or less.

6. A compound or pharmaceutically acceptable salts thereof according to claim 1 wherein said compound also inhibits TNF-alpha production resulting from LPS stimulation in macrophages in the presence of plasma with an EC₅₀ of about 100 μM or less.

7. A compound or pharmaceutically acceptable salts thereof according to claim 1 wherein said compound also inhibits IL-1 production resulting from LPS stimulation in macrophages in the presence of plasma with an EC₅₀ of about 100 μM or less.

8. A compound or pharmaceutically acceptable salts thereof according to claim 1 wherein said compound also inhibits LPS induced TNF-alpha in a mouse with an ED₅₀ of about 100 mg/kg or less.

9. A compound or pharmaceutically acceptable salts thereof according to claim 1 wherein said compound also inhibits LPS induced IL-1 in a mouse with an ED50 of about 100 mg/kg or less.

10. A compound or pharmaceutically acceptable salts thereof according to claim 1 wherein said compound also inhibits collagen induced arthritis in a mouse with an ED₅₀ of about 500 mg/kg/day or less.

11. A compound or pharmaceutically acceptable salts thereof having an IC₅₀ of about 20 μM or less in a COT phosphorylation assay in macrophages and having a moiety of the formula 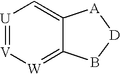
as a component of its complete structure, wherein
A is selected from the group consisting of N, S, O, bond, C═C, C and N;
B is selected from the group consisting of N, S, O, bond, C═C, C and N;
D is selected from the group consisting of C, N, S, O, and C═C;
wherein a double bond is optionally between A and D or B and D;
provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C or N;
V is C or N; and
W is C or N.

12. A compound or pharmaceutically acceptable salts thereof according to claim 11 wherein the moiety is of the formula

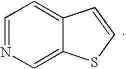

13. A compound or pharmaceutically acceptable salts thereof according to claim 11 wherein the moiety is of the formula

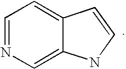

14. A compound or pharmaceutically acceptable salts thereof according to claim 11 wherein the moiety is of the formula

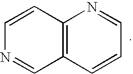

15. A compound or pharmaceutically acceptable salts thereof according to claim 11 wherein the moiety is of the formula

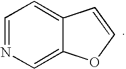

16. A compound or pharmaceutically acceptable salts thereof according to claim 11 wherein the moiety is of the formula

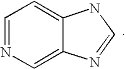

17. A compound or pharmaceutically acceptable salts thereof, having an IC₅₀ of about 5 μM or less in a MK2 HTRF enzyme assay at 5 μM ATP.

18. A compound or pharmaceutically acceptable salts thereof, according to claim 17 wherein said compound also has at least one of the following properties:
a) inhibits formation of phospho-Hsp27 resulting from LPS stimulation in a macrophage with an EC₅₀ of about 10 μM or less;
b) inhibits TNF-alpha production resulting from LPS stimulation in macrophages with an EC₅₀ of about 20 μM or less;
c) inhibits TNF-alpha production resulting from LPS stimulation in macrophages in the presence of plasma with an EC₅₀ of about 100 μM or less;
d) inhibits LPS induced TNF-alpha in a mouse with an ED₅₀ of about 100 mg/kg or less; or
e) inhibits collagen induced arthritis in a mouse with an ED₅₀ of about 500 mg/kg/day or less.

19. A compound or pharmaceutically acceptable salts thereof, according to claim 17 wherein said compound also inhibits formation of phospho-Hsp27 resulting from LPS stimulation in a macrophage with an EC₅₀ of about 10 μM or less.

20. A compound or pharmaceutically acceptable salts thereof, according to claim 17 wherein said compound also inhibits TNF-alpha production resulting from LPS stimulation in macrophages with an EC₅₀ of about 20 μM or less.

21. A compound or pharmaceutically acceptable salts thereof, according to claim 17 wherein said compound also inhibits TNF-alpha production resulting from LPS stimulation in macrophages in the presence of plasma with an EC₅₀ of about 100 μM or less.

22. A compound or pharmaceutically acceptable salts thereof, according to claim 17 wherein said compound also inhibits LPS induced TNF-alpha in a mouse with an ED₅₀ of about 100 mg/kg or less.

23. A compound or pharmaceutically acceptable salts thereof, according to claim 17 wherein said compound also inhibits collagen induced arthritis in a mouse with an ED₅₀ of about 500 mg/kg/day or less.

24. A compound or pharmaceutically acceptable salts thereof, having an IC₅₀ of about 10 μM or less in a MK2 HTRF enzyme assay at 10 μM ATP and having a moiety of the formula 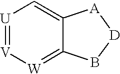
as a component of its complete structure, wherein
A is selected from the group consisting of N, S, O, bond, C═C, C and N;
B is selected from the group consisting of N, S, O, bond, C═C, C and N;
D is selected from the group consisting of C, N, S, O, and C═C;
wherein a double bond is optionally between A and D or B and D;
provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C or N;
V is C or N; and
W is C or N.

25. A compound of formula (I), 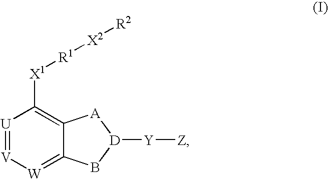
pharmaceutically acceptable salts thereof, metabolites thereof, isomers thereof, or pro-drugs thereof, wherein
A is selected from N, S, O, bond, C═C, C(J), C(J)₂ and N(J);
B is selected from N, S, O, bond, C═C, C(J), C(J)₂ and N(J);
D is selected from C, N, S, O, and C═C;
wherein a double bond is optionally between A and D or B and D;
provided that A, B and D are not each S at the same time, not all O at the same time, not all C═C at the same time, not S—O—S or not O—S—O;
further provided that A and B are not bonds at the same time, A-D or B-D are not S—S, and A-D or B-D are not O—O;
U is C(J) or N;
V is C(J) or N;
W is C(J) or N;
provided that U, V and W are not all N at the same time;
J for each occurrence is independently H or halogen or is an optionally substituted moiety selected from Y-Z, —OR³, —S(R³), —S(O)R³, —S(O)₂R³, —N(R³)SO₂R³, —N(R³)C(O)N(R³)₂, —N(R³)₂, —N(R³)C(O)R³, —N(R³)-aliphatic-O—C(O)-aliphatic, —C(═O)-O-aliphatic-aryl, —C(═O)—O-aliphatic-cycloalkyl, —C(═O)—O-aliphatic-heterocyclyl, -phenyl-N(R³)-aliphatic-aryl, -phenyl-N(R³)-aliphatic-cycloalkyl, -phenyl-N(R³)-aliphatic-heterocyclyl, -phenyl-N(R³)-aliphatic, -phenyl-N(R³)-cycloalkyl, -phenyl-N(R³)-aryl, -phenyl-N(R³)—COOH, heterocyclyl-SO₂—NH-phenyl-, phenylalkoxy and CHO;
Y is selected from a bond, aliphatic, C(═O), C(═N(R³)), C(═N—N(R³)₂), C(═N—OR³), S(O) and S(O₂), NR³—C(═O), C(═O)NR³, N(R³)C(═O)N(R³), and NR³, wherein each of the foregoing groups can optionally be preceded or followed by an optionally substituted aliphatic group;
Z is a, H, halogen, CN, CF₃, N(R³)₂, OR³, 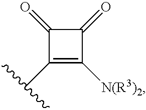
or is independently an optionally substituted moiety selected from aliphatic, aryl, cycloalkyl, heterocyclyl, —(CH₂)ₐ—C(O)—N(R³)₂, —C(O)R³, —C(O)OR³, —C(O)N(R³)₂, —C(O)CF₃, —S(O)R³ and —SO₂R³;
X¹ is a bond, halogen, N(R³), aliphatic, O, S, SO, SO₂, C(═NR³), C(═N—N(R³)₂), N(R³)SO₂, SO₂N(R³), N(R³)C(O)N(R³)S(O), N(R³)(CH₂)ₐN(R³)C(═O), N(R³)C(═O)N(R³), C(O)O, C(O), N(R³)C(O), C(O)N(R³), N(R³)C(O)N(R³), (CH₂)ₐN(R³), N(R³)(CH₂)ₐ, or (CH₂)ₐN(R³)(CH₂)ₐ;
R¹ is a bond, a moiety of formula A, 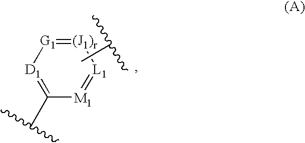
or an an optionally substituted moiety selected from an aliphatic group, benziridazolyl, benzofuranyl, benzoisothiazolyl, benzoisoxazolyl, benzoxazolyl, benzothiazolyl, benzothienyl, cycloaljkyl, 2,3-dihydrobenzofuranyl, 1,1-dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl, 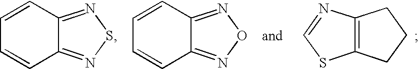
wherein each of the foregoing groups can be optionally substituted by one or more R^{b};
wherein when r is 1 then D₁, G₁, J₁, L₁ and M₁ are each independently selected from CR^{b} and N, provided that at least two of D₁, G₁, J₁, L₁ and M₁ are CR^{b}; or
when r is 0, then one of D₁, G₁, L₁ and M₁ is NR^{b}, one of D₁, G₁, L₁ and M₁ is CR^{b} and the remainder are independently selected from CR^{b}, S, O and N;
when R¹ is not a bond then X² is a bond, aliphatic, N(R³), O, S, SO, SO₂, C(═NR³), C(═N—N(R³)₂), N(R³)SO₂, SO₂N(R³), N(R³)C(O)N(R³)S(O), N(R³)(CH₂)ₐN(R³)C(═O), N(R³)C(═O)N(R³), C(O)O, C(O), N(R³)C(O), N(R³)C(O)N(R³), C(O)N(R³), (CH₂)ₐN(R³), N(R³)(CH₂)ₐ, or (CH₂)ₐN(R³)(CH₂)ₐ;
or when R¹ is a bond then X² is a bond and R² is not a bond;
R² is a bond, R³, a moiety of formula B, 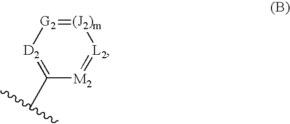
or an an optionally substituted moiety selected from an aliphatic group, benzimidazolyl, benzofuranyl, benzoisothiazolyl, benzoisoxazolyl, benzoxazolyl, benzothiazolyl, benzothienyl, cycloalkyl, 2,3-dihydrobenzofuranyl, 1,1-dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl, 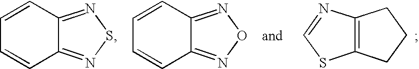
wherein each of the foregoing groups can be optionally substituted by one or more R^{b};
wherein when m is 1 then D₂, G₂, J₂, L₂ and M₂ are each independently selected from CR^{d} and N, provided that at least two of D₂, G₂, J₂, L₂ and M₂ are CR^{d}; or
when m is 0, then one of D₂, G₂, L₂ and M₂ is NR^{d}, one of D₂, G₂, L₂ and M₂ is CR^{d} and the remainder are independently selected from CR^{d}, S, O and N;
R^{b} and R^{d} is an optionally substituted cycloalkyl or heterocyclyl ring fused with the ring to which it is attached;
or R^{b} and R^{d} for each occurrence is independently hydrogen, a halogen, —OR³, NO₂, OCF₃, OH, CF₃, CN, C(O)H, C(O)OH, OCH₃ or is selected from an aliphatic, alkoxy, aliphatic-C(O)—, aliphatic-O(O)C—, aliphatic-S—, aliphatic-S(O)ₚ—, amido groups, amino, aminoalkoxy, aryl, arylalkoxy-, arylaliphatic-, aryloxy, aryl-C(O)—, aryl-O(O)C—, aryl-S—, aryl-S(O)ₚ—, aryl-aliphatic-S—, carboxamido, cycloalkyl, cycloalkyl-alkoxy, cycloalkyloxy, cycloalkyl-aliphatic-, cycloalkyl-C(O)—, cycloalkyl-O(O)C—, cycloalkyl-S—, cycloalkyl-aliphatic-S—, cycloalkyl-S(O)ₚ—, heterocyclyl, heterocycloalkoxy, heterocyclo-aliphatic, heterocyclyloxy, heterocyclyl-C(O)—, heterocyclyl-O(O)C—, heterocyclo-S—, heterocyclo-S(O)ₚ—, heterocyclo-aliphatic-S—, CF₃-carbonylamino, CF₃-sulfonamido, -Z¹-C(O)N(R³)₂, -Z¹-N(R³)—C(O)—R⁴, -Z¹-N(R³)—S(O)₂—R⁴, -Z¹-N(R³)—C(O)—N(R³)—R⁴, —N(R³)—C(O)R³, —N(R³)—C(O)OR³, —O—R⁴—C(O)-heterocyclyl-OR³, R^{e} and —CH₂OR^{e}, wherein each of the foregoing groups can be optionally substituted;
p is 1 or 2;
R^{e} for each occurrence is independently hydrogen, optionally substituted aliphatic, optionally substituted heterocyclyl, —(C₁-C₈)—N^{f}R^{g}, -Q-(CH₂)ₜ—NR^{f}R^{g}, -Q-(CH₂)ₜ—O-alkyl, -Q-(CH₂)ₜ—S-alkyl or -Q-(CH₂)ₜ—OH;
R^{f} and R^{g} are each independently H, an aliphatic group, alkanoyl or SO₂-alkyl;
or R^{f}, R^{g} and the nitrogen atom to which they are attached together form a five- or six-membered heterocyclic ring;
t is an integer from 2 to 6;
Q is a bond, O, S, S(O), S(O)₂, or NR^{h};
Rh is H or an aliphatic group;
Z¹ for each occurrence is independently a covalent bond or an aliphatic group;
R³ for each occurrence is H, CN, CF₃, or is independently selected from the optionally substituted group aliphatic, cycloalkyl, aryl, heterocyclyl, (CH₂)ₐC(O)N(R⁴)₂, —OR⁴, —C(O)R⁴, —C(O)OR⁴, —C(O)N(R⁴)₂, —C(O)CF₃, —S(O)R⁵ and —SO₂R⁵;
a is an integer from 1 to 5;
R⁴ for each occurrence is H or an optionally substituted moiety independently selected from aliphatic, aryl-aliphatic, cycloalkyl-aliphatic, heterocyclyl-aliphatic, cycloalkyl, heterocyclyl and aryl;
R⁵ is H or CF₃ or is an optionally substituted moiety selected from aliphatic, aryl and heterocyclyl;
provided that:
when U is CH; V is N; W is CH; B is S; A is CH; D is C; X¹ is O, S(O)ₙ, C═CH₂, CH—CH, CH(OH), C(═O), C(═O)NH, OCH₂, CH₂, or C(OH)(CH₂OH), wherein n is 0, 1 or 2; then R¹—X²—R² is not phenyl, cycloalkyl, pyridinyl, furanyl or 1,3,4-thiadiazolyl;
each of which can be optionally substituted by one or more halogen, optionally substituted alkyl, optionally substituted alkenyl, COOR, NHC(═O)R, C(═O)NHR, COR, NH₂, CN, 1-pyrazolyl or alkoxy;
wherein R is H or is selected from alkyl, alkylaryl and morpholinyl, wherein each of the foregoing groups can be optionally substituted;
when U is CH; V is N; W is CH; B is S; A is CH; D is C; then X¹—R¹—X²—R²is not H, Cl, Br, or —SCH₂C(═O)NH₂;
when U is CH; V is N; W is CH; B is S; A is CH; D is C; then U is not C(J) wherein J is 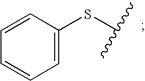
when U is CH; V is N; W is CH; B is S; A is CH; D is C; X¹ is O, S(O)ₙ, C═O, or NCH₃, wherein n is 0, 1 or 2;
or when U is CH; V is N; W is CH; B is S; A is CH; D is C; X¹—R¹—X²—R² is morpholinyl; then Y-Z is not
—C(═O)—NH—CH₃,
—C(═O)—N(CH₃)₂,
—C(═O)—NH—(CH₂)₂OH,
—C(═O)—NH—CH(CH₃)—C(═O)—OH,
—C(═O)—NH—CH₂OH,
—C(═O)—NH—CH₂—C(═O)—OCH₃,
—C(═O)—NH—CH₂—C(═O)—NH₂,
—C(═O)═NH—CH₂—CHO,
—C(═O)—CH(CH₃)—C(═O)—NHCH₃,
—C(═O)—CH₂—CN,
—CH═CH—C(═O)—NH₂,
—CH(OH)—CH(OH)—C(═O)—NHCH₃,
—C═N(CH₃)NH₂,
—C═N(H)—OCH₃,
—O-phenyl wherein the phenyl is substituted by —CH₂═CH₂—C(═O)—OH, —CH₂OH or —NH—C(═O)—O—C(CH₃)₃,
—C(═O)-phenyl-morpholinyl,
oxadiazolyl optionally substituted by NH₂, S, CH₃, —NH—CH₃, or phenyl,
triazolyl optionally substituted by CH₃ or CH₃ and NH₂,
isoxazolyl substituted with NH₂, 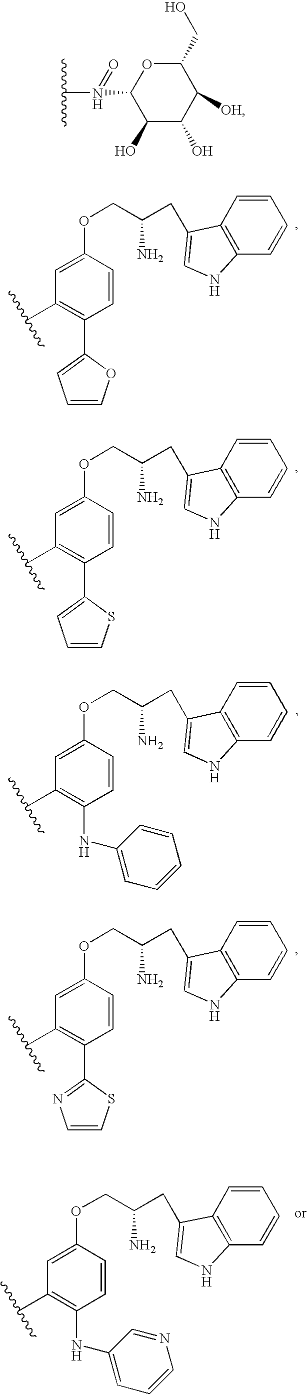 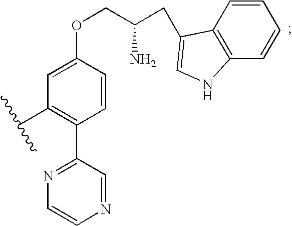
when U is CH, V is N, W is CH, B is S, A is CH, D is C, and X¹ is NH then Y-Z is not —C(═O)—NH—CH₃ or 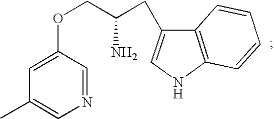
when U is CH; V is N; W is CH; B is S; A is CH; D is C; Y-Z is C(═O)NH₂ then X¹—R¹—X²—R²is not 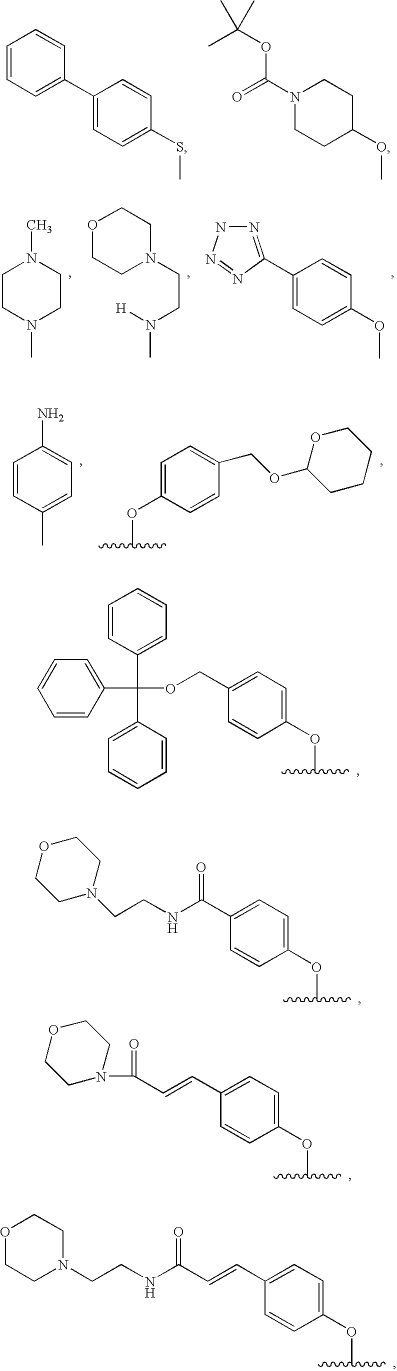 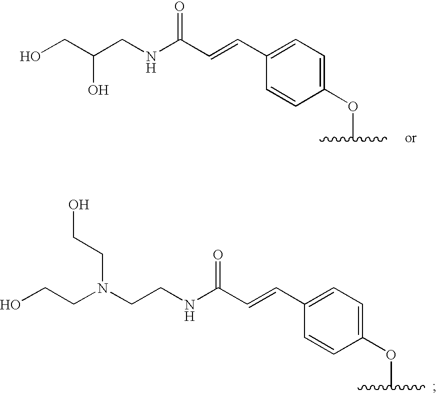
when U is CH; V is N; W is CH; B is S; A is CH; D is C; then X¹—R¹—X²—R² is not phenyl optionally substituted with one or more CF₃, Cl or F; and
when A is C(J); D is C; and there is a double bond between A and D; B is S; U is N; V is C;
W is C; Y-Z is H, methyl, ethyl or propyl; X¹—R¹—X²—R² is —NH₂; then J is not a substituted phenyl;
when Formula (I) is 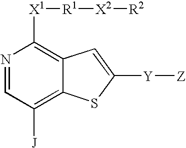
wherein
J is NH₂ or NHCH₃;
Y is a bond;
Z is selected from phenyl, 4,5,6,7-tetrahydrobenzo[b]thienyl, 1,3-dihydrobenzo[c]isothiazolyl, cyclohexyl, cyclopentyl, ethyl, imidazolyl, methyl, furanyl, pyrazolyl, pyridinyl, pyrrolidinyl, pyrrolyl, tetrahydrofuranyl, thiazolyl, thienyl, and thiomorpholine 1,1-dioxide, any of which can be optionally substituted or Z is —CH═CH—CH₃;
then X¹—R¹—X²—R² is not —C(═O)NH₂;
when Formula (I) is 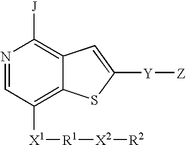
wherein
J is —C(═O)NH₂;
Y is a bond;
Z is cyclohexyl, thiazolyl or optionally substituted phenyl;
then X¹—R¹—X²—R² is not N₂ or NHCH₃; Y is a bond;
Z is selected from phenyl, 4,5,6,7-tetrahydrobenzo[b]thienyl, 1,3-dihydrobenzo[c]isothiazolyl, cyclohexyl, cyclopentyl, ethyl, imidazolyl, methyl, furanyl, phenyl, pyrazolyl, pyridinyl, pyrrolidinyl, pyrrolyl, tetrahydrofuranyl, thiazolyl, thienyl, and thiomorpholine 1,1-dioxide, any of which can be optionally substituted or Z is —CH═CH—CH₃;
then J is not —C(═O)NH₂;
a compound of Formula (I) is not 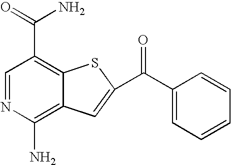
a compound of Formula (I) is not 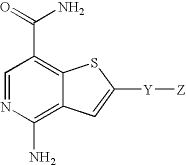
wherein Y is ethyl or propyl and Z is phenyl or OCH₃;
a compound of Formula (I) is not 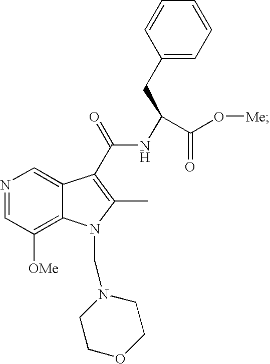
a compound of Formula (I) is not 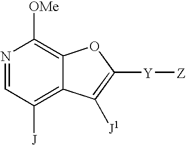
wherein
J is —C(═O)—NH—R wherein R is selected from phenyl, pyrazolyl, pyridyl, isoxazolyl and pyridinyl and can be optionally substituted;
J¹ is H, pyridinyl or tetrahydrofuranyl;
Y is —C(═O) or a bond; and
Z is methyl, ethyl, tetrahydropyranyl, OCH₃ or optionally substituted piperidinyl;
then X¹—R¹—X²—R² is not OCH₃;
a compound of Formula (I) is not 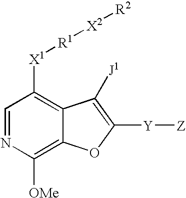
wherein
X¹—R¹—X²—R² is —C(═O)—NH—R³ wherein R³ is selected from phenyl, pyrazolyl, pyridyl, isoxazolyl and pyridinyl and can be optionally substituted;
Y is —C(═O) or a bond;
Z is methyl, ethyl, tetrahydropyranyl, OCH₃ or optionally substituted piperidinyl; and
J¹ is H, pyridinyl or tetrahydropyranyl;
a compound of Formula (I) is not 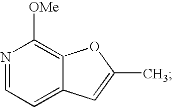
a compound of Formula (I) is not 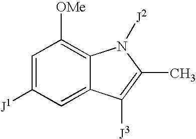
wherein J¹ is Cl, F or H;
J² is —CH₂-phenyl wherein the phenyl is optionally substituted, —CH₂CH₂CH₂-piperazinyl wherein the piperazinyl is optionally substituted, —CH₂—CH₂-morpholinyl or CH₂—CH₂—CH₂-morpholinyl;
J³ is optionally substituted and is selected from —C(═O)—NH-cyclopentyl, —C(═O)—NH-cyclohexyl, —C(═O)—NH—CH₂CH₃, —C(═O)—NH-1,3,3-trimethylbicyclo[2.2. 1]heptan-2-yl, —C(═O)—NH—CH(—CH₂-phenyl)-CO₂CH₃, —C(═O)—NH—CH(CO₂CH₃)—CH₂-phenyl, —C(═O)—NH—CH₃, —C(═O)—NH-phenyl, —C(═O)-tetrahydroquinolinyl, —C(═O)—NH—CH(CH₃)—CH₂—CH₃, —C(═O)—NH—CH(—CH₂-phenyl)-CO₂CH₃, —C(═O)—NH—CH(—CH₂-phenyl)-oxazolyl; —C(═O)—NH—CH(—CH₂-phenyl)-C(═O)—NH₂, —C(═O)—NH—CH(—CH₂-phenyl)-C(═O)—N(CH₃)(OCH₃), —C(═O)—NH—CH(‘3CH₂-phenyl)-[1,2,4]oxadiazolyl, —C(═O)—NH—CH(—CH₂—CH₂—S—CH₃)—C(═O)—OCH₃, —C(═O)—NH—CH(CH(CH₃)₂)—C(═O)—OCH₃, —C(═O)═NH—CH(—CH₂-phenyl)-C(═O)—OC(CH₃)₃, —C(═O)—NH—CH—(CH₂-phenyl)-C(═O)—OCH₂CH₃, —C(═O)—NH—CH(CH₃)-phenyl, —C(═O)—NH—CH(—CH₂-thienyl)-C(═O)—OCH₃, —C(═O)═NH—CH(thiazolyl)-C(═O)—OCH₃, and —C(═O)—NH—CH(—CH₂-phenyl)-tetrazolyl;
a compound of Formula (I) is not 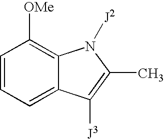
wherein
J¹ is selected from H, pentyl, —CH₂—CH₂-piperidinyl, —CH₂—CH₂—OCH₃, —CH₂-pyridinyl, —CH₂—CH₂—CH₂-morpholinyl, —CH₂—CH₂—N(CH₃)₂, —CH₂—CH₂-pyrrolidinyl, —N(CH₂CH₃)₂, —CH₂—CH₂-cyclohexyl, —CH₂—CH₂—N(CH(CH₃)₂), —CH₂—CH₂—OCH₂CH₃, —CH₂—CH₂—CH₂—OCH₂-phenyl, —CH₂-tetrahydrofuranyl, —CH₂—CH₂-morpholinyl wherein the morpholinyl is optionally substituted, —CH₂—CH₂—O-phenyl, —C(═O)—O—C(CH₃)₃, and COOH; and
J² is —C(═O)—NH-1,3,3-trimethylbicyclo[2.2.1]heptan-2-yl or —CH₂—CH₂-morpholinyl;
a compound of Formula (I) is not 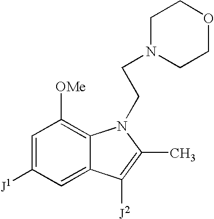
wherein
J¹ is OCH₃ or CH₃;
J² is —C(═O)—NH-1,3,3-trimethylbicyclo[2.2.1]heptan-2-yl or —C(═O)—NH—CH(CO₂CH₃)—CH₂-phenyl;
a compound of Formula (I) is not 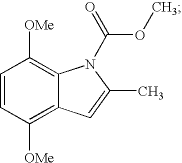
when Formula (I) is 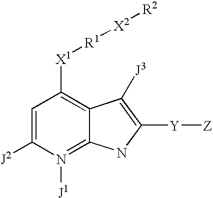
wherein
J¹ is H, OH or —CH₂—CH₂-morpholinyl;
J² is H or —S—CH₂—CH₂—CH₃;
J³ is —C(═O)—NH—CH(—CH₂-phenyl)-CO₂CH₃ or —C(═O)—C(═O)-piperazinyl wherein the piperazinyl is optionally substituted;
then X¹—R¹—X²—R² is not OCH₃;
when formula (I) is 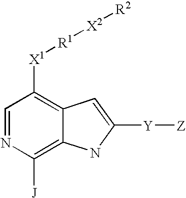
wherein
J is optionally substituted and is selected from 1,2,4-triazolyl, pyrazolyl and pyrazinyl;
Y is a bond;
Z is H or CH₃;
then X¹—R¹—X²—R² is not OCH₃;
when Formula (I) is 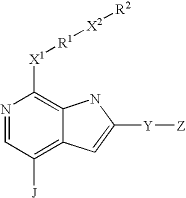
wherein
Y is a bond;
Z is H or CH₃;
X¹—R¹—X²—R² is optionally substituted and is selected from 1,2,4-triazolyl, pyrazolyl or pyrazinyl;
then J is not OCH₃;
a compound of Formula (I) is not 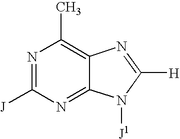
wherein
J is H or CH₃ and J¹ is optionally substituted tetrahydrofuranyl;
a compound of Formula (I) is not 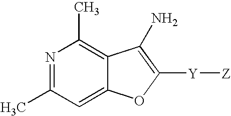
wherein
Y is —C(═O) and Z is optionally substituted phenyl;
a compound of Formula (I) is not 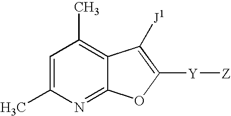
wherein
J¹ is —NH—C(═O)—NH₂, NH₂ or pyridinyl; Y is —C(═O); and
Z is optionally substituted thienyl or optionally substituted phenyl;
a compound of Formula (I) is not 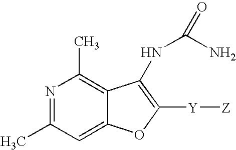
wherein
Y is —C(═O) and Z is phenyl substituted with two methyls;
when Formula (I) is 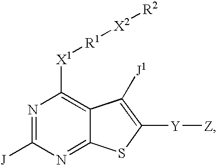
wherein
J is selected from H, —NH—C(═O)═NH—CH(C(C═O)OH)—CH₂—CH₂)CH₃)₂, CH₃, isopropyl, —NH—CH₂—CH₃ and —NH—CH₂—CH₂OH;
J¹ is selected from cyclohexyl, cyclopentyl, pyridinyl and optionally substituted phenyl;
Y is a bond;
Z is selected from pyridinyl, pyridazinyl, pyrimidinyl, cyclohexyl, cyclopentyl and optionally substituted phenyl;
then X¹—R¹—X²—R² is not —NH-ethyl wherein the ethyl is optionally substituted with OH;
a compound of Formula (I) is not 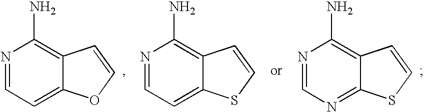
a compound of Formula (I) is not 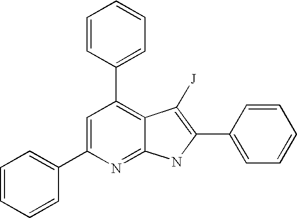
wherein J is —C(═O)—C(═O)-piperazinyl wherein the piperazinyl is substituted;
a compound of Formula (I) is not 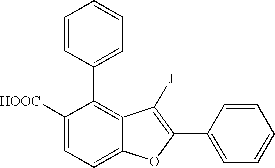
wherein J is H or —C(═O)—OCH₃;
a compound of Formula (I) is not 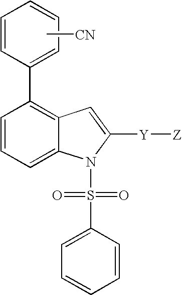
wherein Y is —C(═O) and Z is substituted phenyl;
a compound for Formula (I) is not 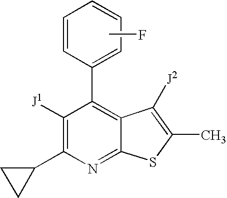
wherein
J¹ is selected from 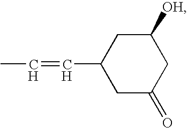
—C(═O)—OCH₃, —CH₂OH, CHO, —CH═CH—CHO, —CH═CH—CH(OH)—CH₂—CH(OH)—CH₂—CO₂CH₂CH₃, —CH═CH—CH(OH)—CH₂—CH(OH)—CO₂Na, —CH═CH—CH(OH)—CH₂—CH(OH)—CO₂Ca, —CH═CH—CH(OH)—CH₂—CH(OH)—CH₂CO₂H, substituted 2,2-dimethyl-1,3-dioxane and —CH═CH—CH₂—CH(OH)—CH₂—C(═O)—CH₂—CO₂₂CH₂CH₃;
J³ is selected from ethyl, substituted benzyl, —CH₂—CH₂—CH(phenyl)-CH₃ and substituted phenyl;
when Formula (I) is 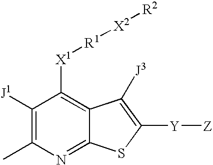
wherein
J¹ is selected from —CH═CH—CH(OH)—CH₂—CH(OH)—CH₂—CO₂CH₂CH₃, 4-hydroxytetrahydropyran-2-one, optionally substituted 2,2-dimethyl-1,3dioxane and —CH═CH—CH(OH)—CH₂—CH(OH)—CH₂CO₂Ca;
J² is selected from —C(CH)═CH₂, cyclopropyl and cyclohexyl;
J³ is selected from —CH═CH-CH₃, n-hexyl, butoxy, 2-pyrimidinyl, 2-thienyl, 2-furanyl, piperazinyl, optionally substituted phenyl, optionally substituted phenoxy and optionally substituted benzyl;
Y is a bond and Z is H,
then X¹—R¹—X²—R² is not phenyl substituted with F or phenyl substituted with F and CH₃;
when Formula (I) is 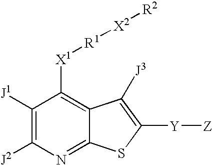
wherein
J¹ is selected from —CHCH—CH(OH)—CH₂—CH(OH)—CO₂CH₂CH₃, —CH═CH—CH(OH)—CH₂—CH(OH)—CH₂—CO₂H, —CH═CH—CH(OH)—CH₂—CH(OH)—CH₂—CO₂Ca, 4-hydroxy-tetrahydropyran-2-one, substituted 2,2-dimethyl-1,3-dioxane, —CH═CH—CH(OH)—CH₂—C(═O)—CH₂CO₂CH₂CH₃, —CH═CH—CH(OH)—CH₂—C(═O)—CH₂—CH₂—CO₂CH₂CH₃, —CH═CH—C(═O)CH₂—C(═O)—CH₂—CO₂CH₂CH₃, and —CH═CH—C(═O)—CH(OH)—CH₂—CO₂CH₂CH₃;
J² is selected from H, isopropyl, methyl, n-propyl, n-hexyl, —C(CH₃)═CH₂ and cyclopropyl;
J³ is selected from H, isopropyl, phenyl, n-propyl, Cl, OCH₃, N(CH₃)₂, benzyl, butyl, ethyl, methyl, isobutyl and cyclopentylmethyl;
Y is a bond; and
Z is selected from methyl, isopropyl, n-propyl, ethyl, n-butyl, Br, Cl, hexyl, —CH═CH₂, phenyl, 2-naphthyl and 3-pyridyl;
then X¹—R¹—X²—R² is not phenyl substituted with F, Cl or CH₃ or phenyl substituted with F and CH₃;
when Formula (I) is 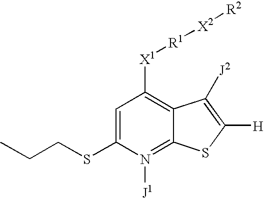
wherein J¹ is OH or H; and
J² is —C(═O)-piperazinyl wherein the piperazinyl is substituted with CH₃ and phenylcarbonyl;
then X¹—R¹—X²—R² is not —OCH₃—CF₃ or OH;
a compound of Formula (I) is not 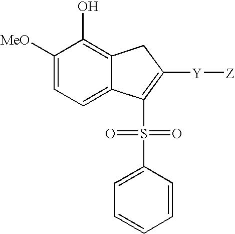
wherein Y is CH₂, —CH(OH) or —C(═O);
Z is isoquinolinyl or Z is phenyl optionally substituted with OH;
a compound of Formula (I) is not 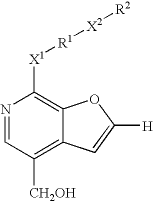
wherein X¹—R¹—X²—R² is —NH-thiazolyl wherein the thiazolyl is optionally substituted with CN;
a compound of Formula (I) is not 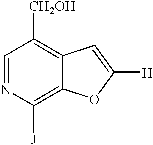
wherein J is is —NH-thiazolyl wherein the thiazolyl is optionally substituted with CN;
a compound of Formula (I) is not 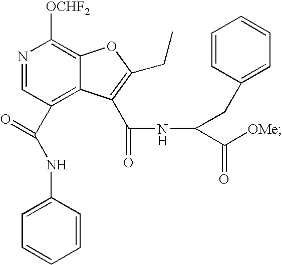
a compound of Formula (I) is not 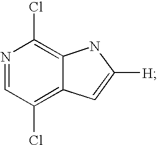
a compound of Formula (I) is not 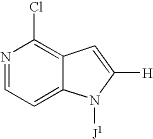
wherein J¹ is H or —C(═O)—N(CH₃)₂;
a compound of Formula (I) is not 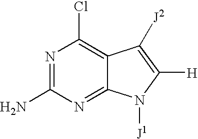
wherein
J¹ is substituted tetrahydrofuranyl and J² is CN, ethyl, CH₃ or H;
a compound of Formula (I) is not 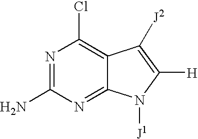
wherein
J¹ is substituted tetrahydrofuranyl and J² is CN, ethyl, methyl or H;
a compound of Formula (I) is not 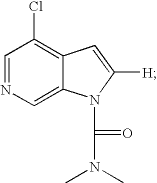
when Formula (I) is 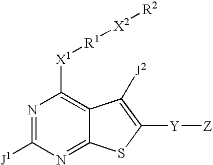
wherein
J¹ is H or CH3;
J² is phenyl substituted with F;
Y is a bond; and Z is pyridazinyl, pyrimidinyl or pyridinyl;
then X¹—R¹—X²—R² is not Cl;
a compound of Formula (I) is not 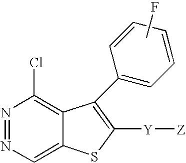
wherein Y is a bond and Z is pyridinyl;
a compound of Formula (I) is not 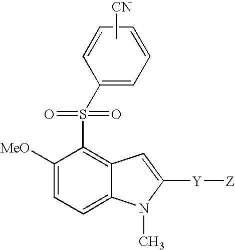
wherein
Y is —C(═O) and Z is optionally substituted phenyl;
when Formula (I) is 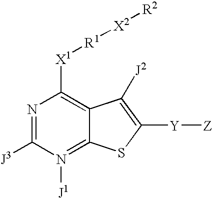
wherein
J¹ is H or OH;
J² is phenyl substituted with F, optionally substituted tetrahydrofuranyl or —C(═O)-piperazinyl wherein the piperazinyl is optionally substituted;
J³ is H or —S-propyl;
Y is a bond; and
Z is pyridinyl, NH₂ or H;
then X¹—R¹—X²—R² is not —NH—CH₂—C(═O)—OCH₂CH₃, —NH—CH₂CH₃, —NH—CH₂—benzo[1,3]dioxazolyl, —NH-benzo[1,3]dioxazolyl, —NH—CH₂-phenyl, —NH—CH₂—CH(CH₂CH₃)₂, —NH—CH₂CH₂—OEt wherein the Et is substituted with OH, —NH—CH₂—C(═O)—NH—CH(CH₂—CH(CH₃)₂)—COOH, —NH—C(═O)—OCH₂CH₃, —NH—CH₂—C(═O)OH or
—NH—CH₂CH₂—OCH₂—CH₂—CH₂OH;
when Formula (I) is 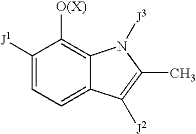
wherein
J¹ is H or OCH₃,
J² is H, —C(═O)—CH(—CH₂-phenyl)-C(═O)—OCH₃ or —C(═O)—NH—C(—CH₂-phenyl)H—C(═O)—OCH₃; and
J³ is H or —CH₂—CH₂-morpholinyl;
then X is not butyl, pentyl or phenyl;
a compound of Formula (I) is not 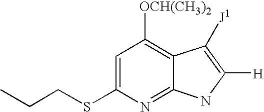
wherein
J¹ is not —C(═O)-piperazinyl wherein the piperazinyl is optionally substituted;
when Formula (I) is 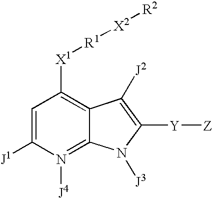
wherein
J¹ is —CH═CH₂ or —S-propyl;
J² is —C(═O)-piperazinyl wherein the piperazinyl is optionally substituted;
J³ is H or —SO₂-phenyl;
J⁴ is H or OH;
Y is a bond; and
Z is optionally substituted phenyl;
then X¹—R¹—X²—R² is not —CH═CH₂ or —S-propyl;
a compound of Formula (I) is not

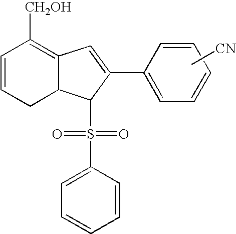

26. A compound or pharmaceutically acceptable salts thereof, metabolites thereof, isomers thereof, or pro-drugs thereof, according to claim 25 wherein, B is S, N or O; X¹ is a bond, O, S or NH.

27. A compound or pharmaceutically acceptable salts thereof, metabolites thereof, isomers thereof, or pro-drugs thereof, according to claim 26 wherein,
U is CH; V is N; W is CH or CNH₂; A is CH; D is C and there is a double bond between A and D; B is S or N;
Y-Z is tetrazole, —C(═O)N(R³)₂, —C(═O)NR³OR³, —NR³C(═O)R³ or —C(═O)OR³;
X¹ is a bond, O or NH; and
R¹ is an optionally substituted group selected from phenyl, benzimidazolyl, benzofuranyl, benzoisothiazolyl, benzoisoxazolyl, benzoxazolyl, benzothiazolyl, benzothienyl, 2,3-dihydrobenzofuranyl, 1,1-dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl, thienyl,

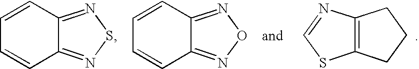

28. A compound or pharmaceutically acceptable salts thereof, metabolites thereof, isomers thereof, or pro-drugs thereof, according to claim 27 wherein, R¹ is phenyl or piperidinyl, both of which can be optionally substituted with R^{b}.

29. A compound or pharmaceutically acceptable salts thereof, metabolites thereof, isomers thereof, or pro-drugs thereof, according to claim 28 wherein Y-Z is a tetrazole, —C(═O)N(R³)₂, —C(═O)NR³OR³ or —C(═O)OR³.

30. A compound or pharmaceutically acceptable salts thereof, metabolites thereof, isomers thereof, or pro-drugs thereof, according to claim 29 wherein
X¹ is NH or a bond;
B is S.

31. A compound or pharmaceutically acceptable salts thereof, metabolites thereof, isomers thereof, or pro-drugs thereof, according to claim 30 wherein
Y-Z is —C(═O)N(H)₂; and
X² is a bond, NH or CH₂ and R² is unsubstituted benzoxazolyl or phenyl optionally substituted with OH, CN, CONH₂ or Br.

32. A compound according to claim 31 wherein the compound is 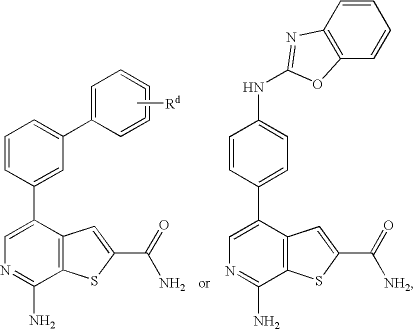
wherein R^{d} is selected from OH, CN, H and CONH₂.
